(19) United States
(12) Patent Application Publication
(10) Pub. No.: US 2022/0165960 A1
(43) Pub. Date: May 26, 2022

(54) HETEROCYCLIC COMPOUND, LIGHT-EMITTING DEVICE INCLUDING THE SAME, AND ELECTRONIC APPARATUS INCLUDING THE LIGHT-EMITTING DEVICE

(71) Applicant: Samsung Display Co., Ltd., Yongin-si (KR)

(72) Inventors: Yeseul LEE, Yongin-si (KR); Hyeongmin KIM, Yongin-si (KR); Heechoon AHN, Yongin-si (KR); Hyunah UM, Yongin-si (KR); Hyoyoung LEE, Yongin-si (KR); Yirang IM, Yongin-si (KR); Seowon CHO, Yongin-si (KR)

(21) Appl. No.: 17/527,396

(22) Filed: Nov. 16, 2021

(30) Foreign Application Priority Data
Nov. 25, 2020 (KR) 10-2020-0160060

(51) Int. Cl. H01L 51/00; C07D 498/04; C07D 513/04; C07F 7/08

(52) U.S. Cl. CPC H01L 51/0071; H01L 51/0072; C07D 498/04; H01L 51/5016; H01L 51/0094; C07F 7/0812; H01L 51/0074; C07D 513/04

(57) ABSTRACT

Provided are a heterocyclic compound represented by Formula 1, a light-emitting device including the same, and an electronic apparatus including the light-emitting device:

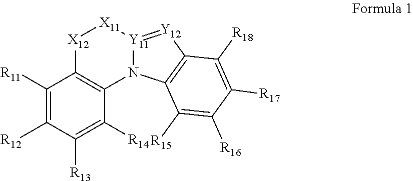

wherein the detailed description of Formula 1 is the same as described in the present specification.

### CROSS-REFERENCE TO RELATED APPLICATION

This application claims priority under 35 U.S.C. § 119 to Korean Patent Application No. 10-2020-0160060, filed on Nov. 25, 2020, in the Korean Intellectual Property Office, the content of which is incorporated by reference herein in its entirety.

### BACKGROUND

### 1. Field

One or more embodiments relate to a heterocyclic compound, a light-emitting device including the same, and an electronic apparatus including the light-emitting device.

### 2. Description of the Related Art

Light-emitting devices are devices that convert electrical energy into light energy. Examples of such light-emitting devices include organic light-emitting devices using organic materials for an emission layer, quantum dot light-emitting devices using quantum dots for an emission layer, and the like.

In a light-emitting device, a first electrode is located on a substrate, and a hole transport region, an emission layer, an electron transport region, and a second electrode are sequentially formed on the first electrode. Holes provided from the first electrode may move toward the emission layer through the hole transport region, and electrons provided from the second electrode may move toward the emission layer through the electron transport region. Carriers, such as holes and electrons, recombine in the emission layer to produce excitons. These excitons transition from an excited state to a ground state to thereby generate light.

### SUMMARY

One or more embodiments include a heterocyclic compound, a light-emitting device including the same, and an electronic apparatus including the light-emitting device.

Additional aspects will be set forth in part in the description, which follows and, in part, will be apparent from the description, or may be learned by practice of the presented embodiments of the disclosure.

According to one or more embodiments, a light-emitting device includes a first electrode,

a second electrode, and

an interlayer located between the first electrode and the second electrode and including an emission layer, wherein

the light-emitting device includes at least one of the heterocyclic compounds represented by Formula 1.

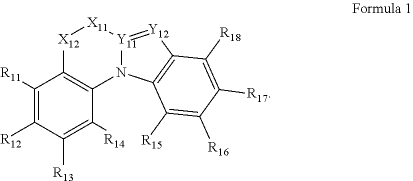

In Formula 1,

i) X₁₁ is O or S and X₁₂ is C(R₁₉)(R₂₀), or ii) X₁₁ is C(R₁₉)(R₂₀) and X₁₂ is O or S,

Y₁₁ and Y₁₂ is N or C,

R₁₁ to R₂₀ are each independently a group represented by *-(L₂₁)ₐ₂₁-(R₂₁)_{b21}, hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂), wherein at least one of R₁₁ to R₂₀ is a group represented by *-(L₂₁)ₐ₂₁-(R₂₁)_{b21},

L₂₁ is a single bond, *—N(R₃₁)—*′, *—Si(R₃₁)(R₃₂)—*′, a C₅-C₆₀ carbocyclic group unsubstituted or substituted with at least one R₁₀ₐ, or a C₁-C₆₀ heterocyclic group unsubstituted or substituted with at least one R₁₀ₐ,

a21 is an integer selected from 1 to 5,

R₂₁ is a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),

b21 is an integer selected from 1 to 5,

R₃₁ and R₃₂ are each independently hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),

R₁₀ₐ is

deuterium (-D), —F, —Cl, —Br, —I, a hydroxyl group, a cyano group, or a nitro group,

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —C(Q₁₁)(Q₁₂)(Q₁₃), —B(Q₁₁)(Q₁₂), —N(Q₁₁)(Q₁₂), —P(Q₁₁)(Q₁₂), —C(═O)(Q₁₁), —S(═O)(Q₁₁), —S(═O)₂(Q₁₁), —P(═O)(Q₁₁)(Q₁₂), —P(═S)(Q₁₁)(Q₁₂), or any combination thereof,

a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —C(Q₂₁)(Q₂₂)(Q₂₃), —B(Q₂₁)(Q₂₂), —N(Q₂₁)(Q₂₂), —P(Q₂₁)(Q₂₂), —C(═O)(Q₂₁), —S(═O)(Q₂₁), —S(═O)₂(Q₂₁), —P(═O)(Q₂₁)(Q₂₂) or —P(═S)(Q₂₁)(Q₂₂), or any combination thereof, or

—Si(Q₃₁)(Q₃₂)(Q₃₃), —C(Q₃₁)(Q₃₂)(Q₃₃), —B(Q₃₁)(Q₃₂), —N(Q₃₁)(Q₃₂), —P(Q₃₁)(Q₃₂), —C(═O)(Q₃₁), —S(═O)(Q₃₁), —S(═O)₂(Q₃₁), —P(═O)(Q₃₁)(Q₃₂), or —P(═S)(Q₃₁)(Q₃₂),

wherein Q₁ to Q₃, Q₁₁ to Q₁₃, Q₂₁ to Q₂₃ and Q₃₁ to Q₃₃ may each independently be hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and

* and *′ each indicate a binding site to a neighboring atom.

Another aspect of the present disclosure provides an electronic apparatus including the light-emitting device and a thin-film transistor,

wherein the thin-film transistor may include a source electrode and a drain electrode, and the first electrode of the light-emitting device may be in electrical contact with one of the source electrode and the drain electrode of the thin-film transistor.

An aspect of the present disclosure provides a heterocyclic compound represented by Formula 1

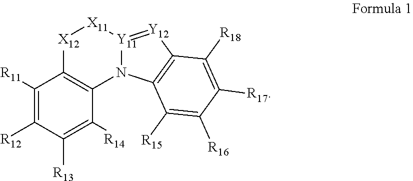

In Formula 1,

i) X₁₁ is O or S and X₁₂ is C(R₁₉)(R₂₀), or ii) X₁₁ is C(R₁₉)(R₂₀) and X₁₂ is O or S,

Y₁₁ and Y₁₂ are each N or C,

R₁₁ to R₂₀ are each independently a group represented by *-(L₂₁)ₐ₂₁-(R₂₁)_{b21}, hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂), wherein at least one of R₁₁ to R₂₀ is a group represented by *-(L₂₁)ₐ₂₁-(R₂₁)_{b21},

L₂₁ is a single bond, *—N(R₃₁)—*′, *—Si(R₃₁)(R₃₂)—*′, a C₅-C₆₀ carbocyclic group unsubstituted or substituted with at least one R₁₀ₐ, or a C₁-C₆₀ heterocyclic group unsubstituted or substituted with at least one R₁₀ₐ,

a21 is an integer selected from 1 to 5,

R₂₁ is a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),

b21 is an integer selected from 1 to 5,

R₃₁ and R₃₂ are each independently hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),

R₁₀ₐ is

deuterium (-D), —F, —Cl, —Br, —I, a hydroxyl group, a cyano group, or a nitro group,

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —C(Q₁₁)(Q₁₂)(Q₁₃), —B(Q₁₁)(Q₁₂), —N(Q₁₁)(Q₁₂), —P(Q₁₁)(Q₁₂), —C(═O)(Q₁₁), —S(═O)(Q₁₁), —S(═O)₂(Q₁₁), —P(═O)(Q₁₁)(Q₁₂), —P(═S)(Q₁₁)(Q₁₂), or any combination thereof,

a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —C(Q₂₁)(Q₂₂)(Q₂₃), —B(Q₂₁)(Q₂₂), —N(Q₂₁)(Q₂₂), —P(Q₂₁)(Q₂₂), —C(═O)(Q₂₁), —S(═O)(Q₂₁), —S(═O)₂(Q₂₁), —P(═O)(Q₂₁)(Q₂₂) or —P(═S)(Q₂₁)(Q₂₂), or any combination thereof, or

—Si(Q₃₁)(Q₃₂)(Q₃₃), —C(Q₃₁)(Q₃₂)(Q₃₃), —B(Q₃₁)(Q₃₂), —N(Q₃₁)(Q₃₂), —P(Q₃₁)(Q₃₂), —C(═O)(Q₃₁), —S(═O)(Q₃₁), —S(═O)₂(Q₃₁), —P(═O)(Q₃₁)(Q₃₂), or —P(═S)(Q₃₁)(Q₃₂),

wherein Q₁ to Q₃, Q₁₁ to Q₁₃, Q₂₁ to Q₂₃ and Q₃₁ to Q₃₃ are each independently hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and

* and *′ each indicate a binding site to a neighboring atom.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic view of a light-emitting device according to an exemplary embodiment;

FIG. 2 is a schematic view of a light-emitting device according to an exemplary embodiment; and

FIG. 3 is a schematic view of a light-emitting device according to an exemplary embodiment.

### DETAILED DESCRIPTION

Reference will now be made in detail to embodiments, examples of which are illustrated in the accompanying drawings, wherein like reference numerals refer to like elements throughout the specification. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Throughout the disclosure, the expression “at least one of a, b or c” indicates only a, only b, only c, both a and b, both a and c, both b and c, all of a, b, and c, or variations thereof.

Because the disclosure may have diverse modified embodiments, embodiments are illustrated in the drawings and are described in the detailed description. An effect and a characteristic of the disclosure, and a method of accomplishing these will be apparent when referring to embodiments described with reference to the drawings. The disclosure may, however, be embodied in many different forms and should not be construed as limited to the embodiments set forth herein.

One or more embodiments of the disclosure will be described below in more detail with reference to the accompanying drawings. Those components that are the same or are in correspondence with each other are rendered the same reference numeral regardless of the figure number, and redundant explanations are omitted.

An expression used in the singular encompasses the expression of the plural, unless it has a clearly different meaning in the context.

It will be further understood that the terms “comprises” and/or “comprising” used herein specify the presence of stated features or elements, but do not preclude the presence or addition of one or more other features or elements.

It will be understood that when a layer, region, or component is referred to as being “on” or “onto” another layer, region, or component, it may be directly or indirectly formed on the other layer, region, or component. That is, for example, intervening layers, regions, or components may be present.

Sizes of elements in the drawings may be exaggerated for convenience of explanation. In other words, because sizes and thicknesses of components in the drawings are arbitrarily illustrated for convenience of explanation, the following embodiments are not limited thereto.

An aspect of the present disclosure provides a heterocyclic compound represented by Formula 1 below

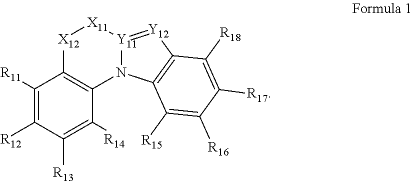

In Formula 1, i) X₁₁ may be O or S and X₁₂ may be C(R₁₉)(R₂₀), or ii) X₁₁ may be C(R₁₉)(R₂₀) and X₁₂ may be O or S, and R₁₉ and R₂₀ are the same as described below.

In one or more embodiments, X₁₁ may be O or S and X₁₂ may be C(R₁₉)(R₂₀).

Y₁₁ and Y₁₂ in Formula 1 may be N or C.

In one or more embodiments, in Formula 1, Y₁₁ and Y₁₂ may each be N; Y₁₁ and Y₁₂ may each be C; or one of Y₁₁ and Y₁₂ may be N and the other one may be C.

In one or more embodiments, one of Y₁₁ and Y₁₂ in Formula 1 may be N and the other may be C.

In one or more embodiments, in Formula 1, Y₁₁ may be C and Y₁₂ may be N.

R₁₁ to R₂₀ in Formula 1 may each independently be a group represented by *-(L₂₁)ₐ₂₁-(R₂₁)_{b21}, hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂), wherein at least one of R₁₁ to R₂₀ is a group represented by *-(L₂₁)ₐ₂₁-(R₂₁)_{b21},

Q₁ to Q₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and

L₂₁, a21, R₂₁, and b21 are the same as described below.

In one or more embodiments, one or two of R₁₁ to R₁₈ in Formula 1 may be a group represented by *-(L₂₁)ₐ₂₁-(R₂₁)_{b21}.

In one or more embodiments, any one of R₁₁ to R₁₈ in Formula 1 may be a group represented by *-(L₂₁)ₐ₂₁-(R₂₁)_{b21}.

In one or more embodiments, R₁₂, R₁₃, R₁₆, or R₁₇ in Formula 1 may be a group represented by *-(L₂₁)ₐ₂₁-(R₂₁)_{b21}.

L₂₁ in Formula 1 may be a single bond, *—N(R₃₁)—*′, *—Si(R₃₁)(R₃₂)—*′, a C₅-C₆₀ carbocyclic group unsubstituted or substituted with at least one R₁₀ₐ, or a C₁-C₆₀ heterocyclic group unsubstituted or substituted with at least one R₁₀ₐ, and

R₃₁ and R₃₂ may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),

wherein Q₁ to Q₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

In one or more embodiments, L₂₁ in Formula 1 may be a single bond, or *—Si(R₃₁)(R₃₂)*′; or

a benzene group, a pentalene group, an indene group, a naphthalene group, an azulene group, a heptalene group, an indacene group, an acenaphthalene group, a fluorene group, a spiro-bifluorene group, a spiro-benzofluorene-fluorene group, a benzofluorene group, a dibenzofluorene group, a phenalene group, a phenanthrene group, an anthracene group, a fluoranthene group, a pyrene group, a chrysene group, a naphthacene group, a picene group, a perylene group, a pyrrole group, a thiophene group, a furan group, a silole group, an imidazole group, a pyrazole group, a thiazole group, an isothiazole group, an oxazole group, an isoxazole group, a pyridine group, a pyrazine group, a pyrimidine group, a pyridazine group, a triazine group, a benzofuran group, a benzothiophene group, a dibenzofuran group, a dibenzothiophene group, a carbazole group, a benzosilole group, a dibenzosilole group, a quinoline group, an isoquinoline group, a benzimidazole group, an imidazopyridine group, or an imidazopyrimidine group, each unsubstituted or substituted with deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a spiro-benzofluorene-fluorenyl group, a benzofluorenyl group, a dibenzofluorenyl group, a phenalenyl group, a phenanthrenyl group, an anthracenyl group, a fluoranthenyl group, a pyrenyl group, a chrysenyl group, a naphthacenyl group, a picenyl group, a perylenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, a benzofuranyl group, a benzothiophenyl group, a dibenzofuranyl group, a dibenzothiophenyl group, a carbazolyl group, a benzosilolyl group, a dibenzosilolyl group, a quinolinyl group, an isoquinolinyl group, a benzimidazolyl group, an imidazopyridinyl group, an imidazopyrimidinyl group, —Si(Q₃₁)(Q₃₂)(Q₃₃), —C(Q₃₁)(Q₃₂)(Q₃₃), —B(Q₃₁)(Q₃₂), —N(Q₃₁)(Q₃₂), —P(Q₃₁)(Q₃₂), —C(═O)(Q₃₁), —S(═O)(Q₃₁), —S(═O)₂(Q₃₁), —P(═O)(Q₃₁)(Q₃₂), —P(═S)(Q₃₁)(Q₃₂), or any combination thereof,

R₃₁ and R₃₂ may each independently be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a spiro-benzofluorene-fluorenyl group, a benzofluorenyl group, a dibenzofluorenyl group, a phenalenyl group, a phenanthrenyl group, an anthracenyl group, a fluoranthenyl group, a pyrenyl group, a chrysenyl group, a naphthacenyl group, a picenyl group, a perylenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, a benzofuranyl group, a benzothiophenyl group, a dibenzofuranyl group, a dibenzothiophenyl group, a carbazolyl group, a benzosilolyl group, a dibenzosilolyl group, a quinolinyl group, an isoquinolinyl group, a benzimidazolyl group, an imidazopyridinyl group, an imidazopyrimidinyl group, —Si(Q₃₁)(Q₃₂)(Q₃₃), —C(Q₃₁)(Q₃₂)(Q₃₃), —B(Q₃₁)(Q₃₂), —N(Q₃₁)(Q₃₂), —P(Q₃₁)(Q₃₂), —C(═O)(Q₃₁), —S(═O)(Q₃₁), —S(═O)₂(Q₃₁), —P(═O)(Q₃₁)(Q₃₂), or —P(═S)(Q₃₁)(Q₃₂), and

Q₃₁ to Q₃₃ may each independently be a C₁-C₁₀ alkyl group, a C₁-C₁₀ alkoxy group, a phenyl group, a phenyl group substituted with a cyano group, a biphenyl group, a terphenyl group, or a naphthyl group.

In one or more embodiments, L₂₁ in Formula 1 may be a single bond, or a group represented by one of Formulae 3-1 to 3-39:

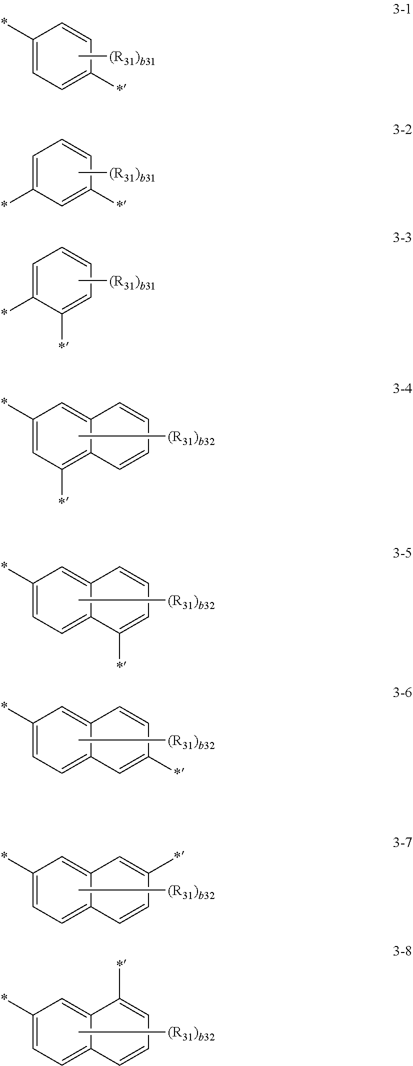

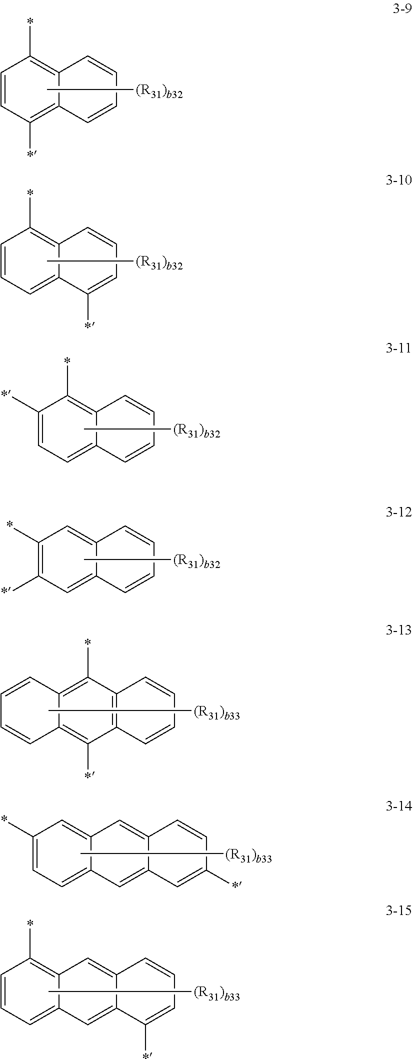

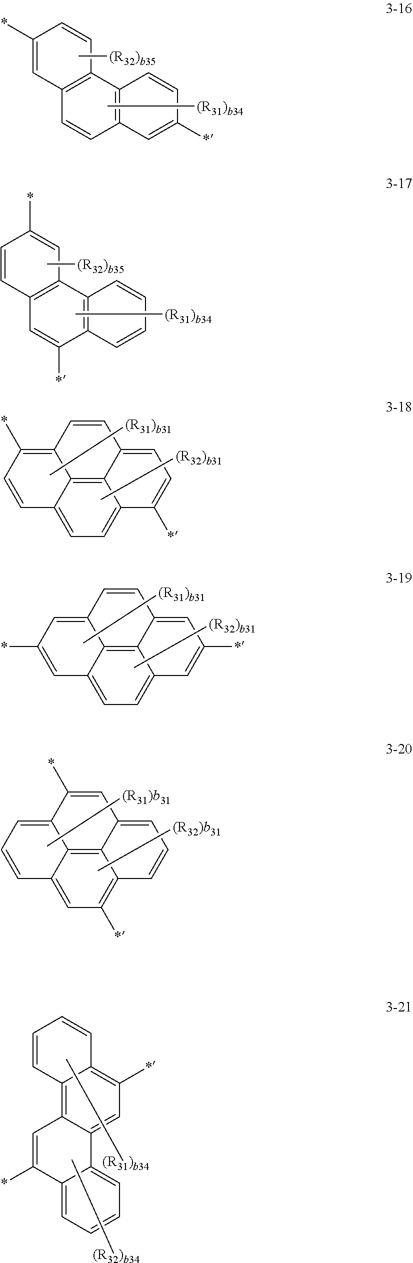

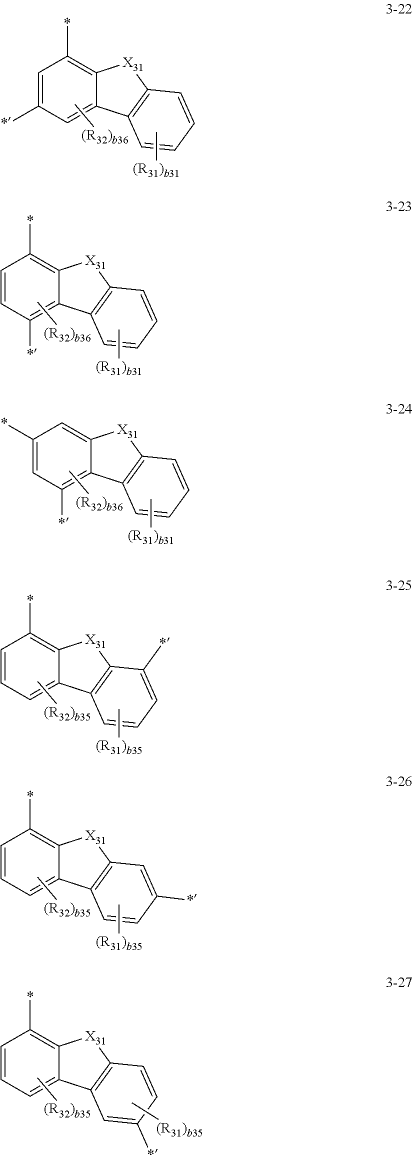

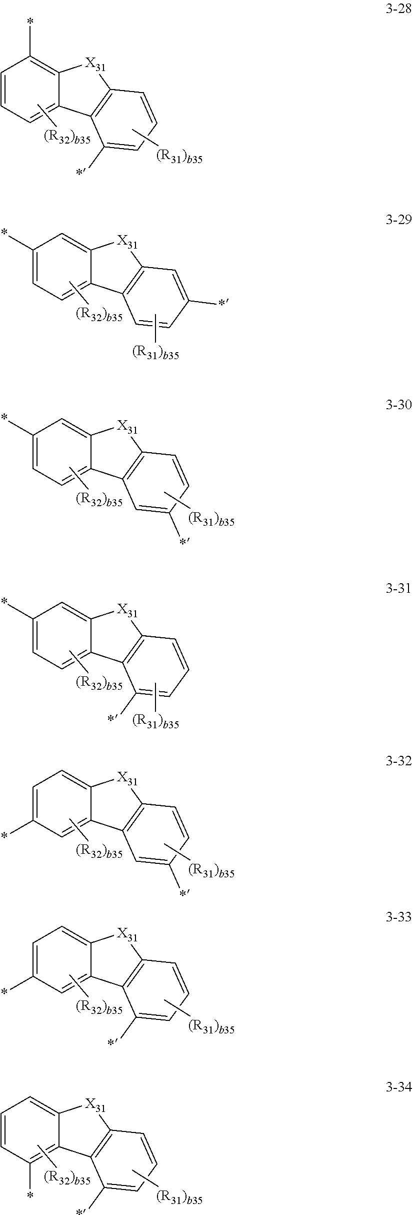

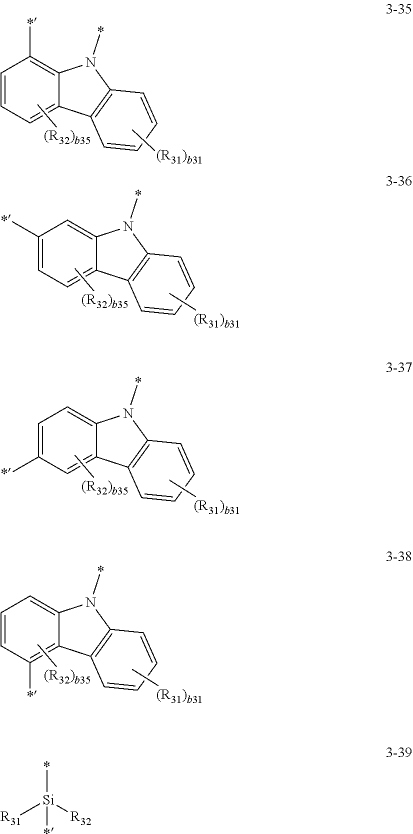

wherein, in Formulae 3-1 to 3-39,

X₃₁ may be O, S, N(R₃₃), or C(R₃₃)(R₃₄),

R₃₁ to R₃₄ may each independently be hydrogen, deuterium, —F, —Cl, —Br, —I, a hydroxyl group, a cyano group, a nitro group, an amidino group, a hydrazino group, a hydrazono group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a carbazolyl group, a dibenzofuranyl group, a dibenzothiophenyl group, a triazinyl group, a benzimidazolyl group, a phenanthrolinyl group, —Si(Q₃₁)(Q₃₂)(Q₃₃), —C(Q₃₁)(Q₃₂)(Q₃₃), —N(Q₃₁)(Q₃₂), or —B(Q₃₁)(Q₃₂),

Q₃₁ to Q₃₃ may each independently be a C₁-C₁₀ alkyl group, a C₁-C₁₀ alkoxy group, a phenyl group, a phenyl group substituted with a cyano group, a biphenyl group, a terphenyl group, or a naphthyl group,

b31 may be an integer selected from 0 to 4,

b32 may be an integer selected from 0 to 6,

b33 may be an integer selected from 0 to 8,

b34 may be an integer selected from 0 to 5,

b35 may be an integer selected from 0 to 3,

b36 may be an integer selected from 0 to 2, and

* and *′ each indicate a binding site to a neighboring atom.

In one or more embodiments, L₂₁ in Formula 1 may be a single bond, or a group represented by one of Formulae 4-1 to 4-6:

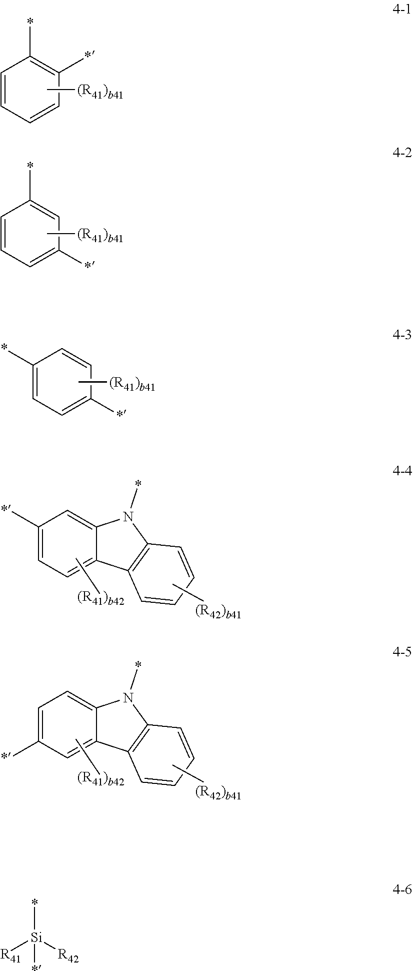

In Formulae 4-1 to 4-6,

R₄₁ and R₄₂ may each independently be hydrogen, deuterium, —F, —Cl, —Br, —I, a cyano group, a methyl group, an ethyl group, an n-propyl group, an isopropyl group, an n-butyl group, a sec-butyl group, an isobutyl group, a tert-butyl group, a phenyl group, a biphenyl group, a terphenyl group, or a carbazolyl group;

b41 may be an integer selected from 0 to 4,

b42 may be an integer selected from 0 to 3, and

* and *′ each indicate a binding site to a neighboring atom.

a21 in Formula 1 may be an integer selected from 1 to 5. When a21 is 2 or more, a plurality of L₂₁ (s) may be identical to or different from each other.

In one or more embodiments, a21 in Formula 1 may be 1, 2, or 3.

R₂₁ in Formula 1 may be a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),

wherein Q₁ to Q₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

In one or more embodiments, R₂₁ in Formula 1 may be a cyclopentenyl group, a cyclohexenyl group, a phenyl group, a biphenyl group, a terphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, an indolyl group, an isoindolyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a dibenzofuranyl group, a dibenzothiophenyl group, a dibenzosilolyl group, a carbazolyl group, a benzocarbazolyl group, a dibenzocarbazolyl group, an azafluorenyl group, an azaspiro-bifluorenyl group, a dibenzothiophenylfluorenyl group, an azacarbazolyl group, a diazacarbazolyl group, an azadibenzofuranyl group, an azadibenzothiophenyl group, an azadibenzosilolyl group, an imidazopyridinyl group, or an imidazopyrimidinyl group, each unsubstituted or substituted with deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkyl group substituted with at least one phenyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, an indolyl group, an isoindolyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a dibenzofuranyl group, a dibenzothiophenyl group, a dibenzosilolyl group, a carbazolyl group, a benzocarbazolyl group, a dibenzocarbazolyl group, an azafluorenyl group, an azaspiro-bifluorenyl group, a dibenzothiophenylfluorenyl group, an azacarbazolyl group, a diazacarbazolyl group, an azadibenzofuranyl group, an azadibenzothiophenyl group, an azadibenzosilolyl group, an imidazopyridinyl group, an imidazopyrimidinyl group, —Si(Q₃₁)(Q₃₂)(Q₃₃), —C(Q₃₁)(Q₃₂)(Q₃₃), —N(Q₃₁)(Q₃₂), —B(Q₃₁)(Q₃₂), or any combination thereof; or

—Si(Q₁)(Q₂)(Q₃), —C(Q₁)(Q₂)(Q₃), —N(Q₁)(Q₂), or —B(Q₁)(Q₂),

wherein Q₁ to Q₃ and Q₃₁ to Q₃₃ may each independently be a C₁-C₁₀ alkyl group, a C₁-C₁₀ alkoxy group, a phenyl group, a phenyl group substituted with a cyano group, a biphenyl group, a terphenyl group, or a naphthyl group.

In one or more embodiments, R₂₁ in Formula 1 may be a group represented by one of Formulae 5-1 to 5-20:

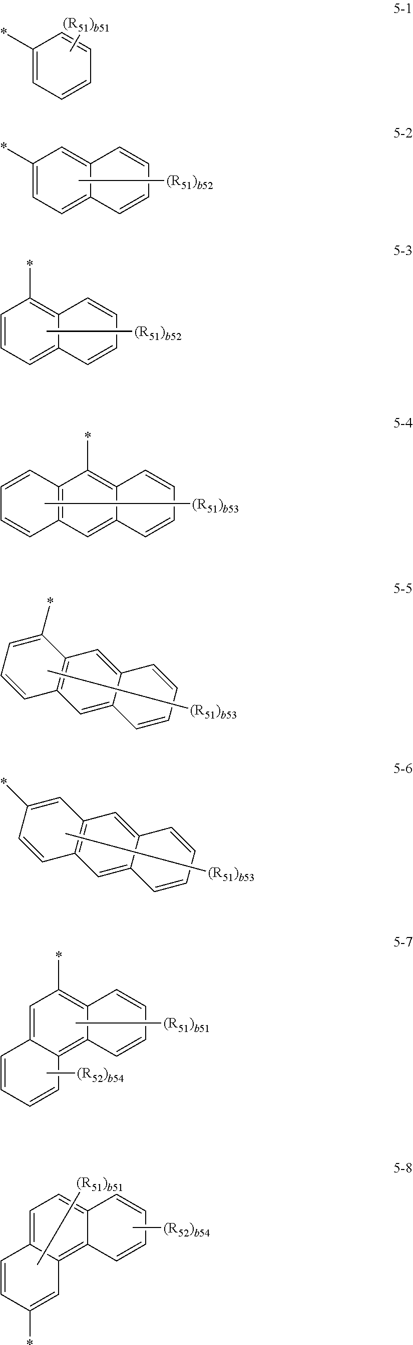

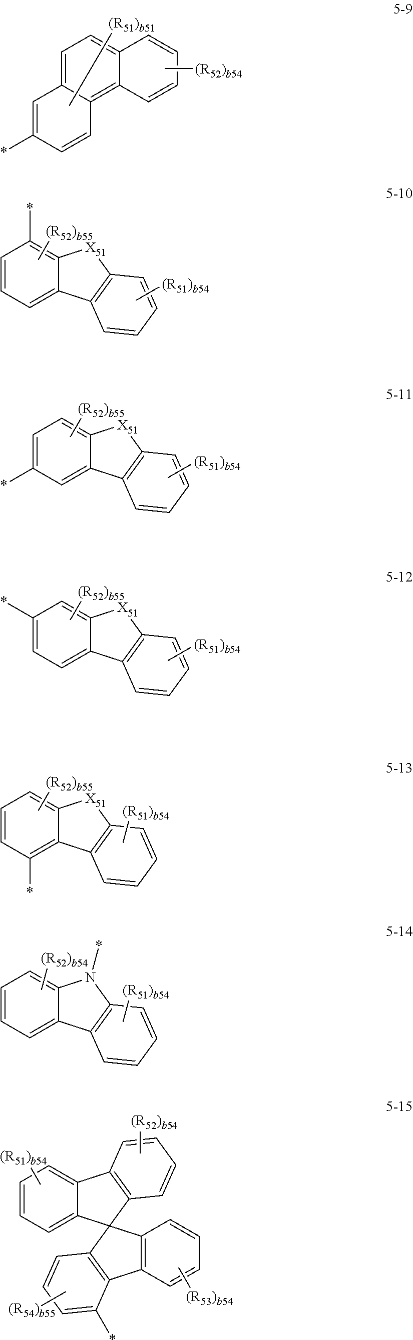

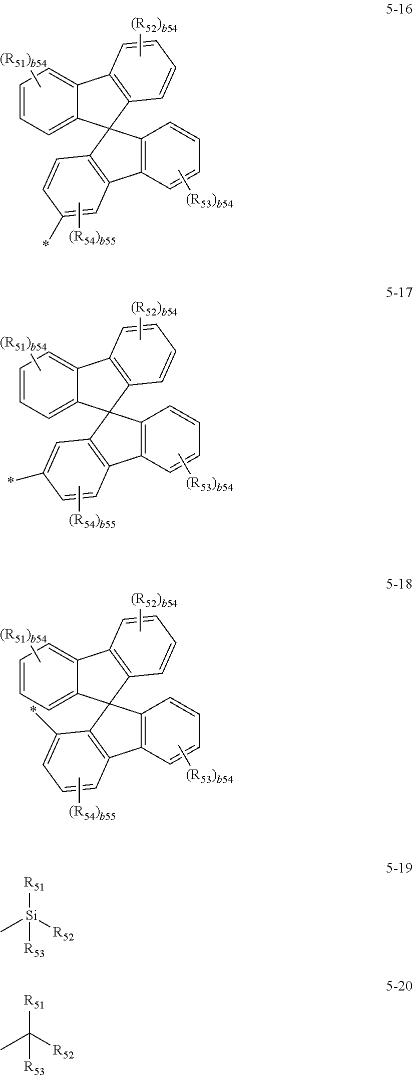

wherein, in Formulae 5-1 to 5-20,

X₅₁ may be O, S, N(R₅₃), or C(R₅₃)(R₅₄),

R₅₁ to R₅₄ may each independently be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkyl group substituted with at least one phenyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyridinyl group, a pyrimidinyl group, a pyrazinyl group, a triazinyl group, a quinolinyl group, an isoquinolinyl group, a benzoquinolinyl group, a naphthyridinyl group, a quinoxalinyl group, a quinazolinyl group, a carbazolyl group, a phenanthridinyl group, an acridinyl group, a phenanthrolinyl group, a phenazinyl group, a dibenzofuranyl group, a dibenzothiophenyl group, a dibenzosilolyl group, —Si(Q₃₁)(Q₃₂)(Q₃₃), —C(Q₃₁)(Q₃₂)(Q₃₃), —N(Q₃₁)(Q₃₂), or —B(Q₃₁)(Q₃₂),

b51 may be an integer selected from 1 to 5,

b52 may be an integer selected from 1 to 7,

b53 may be an integer selected from 1 to 9,

b54 may be an integer selected from 1 to 4,

b55 may be an integer selected from 1 to 3,

Q₃₁ to Q₃₃ may each independently be a C₁-C₁₀ alkyl group, a C₁-C₁₀ alkoxy group, a phenyl group, a phenyl group substituted with a cyano group, a biphenyl group, a terphenyl group, or a naphthyl group, and

* indicates a binding site to an adjacent atom.

In one or more embodiments, R₂₁ in Formula 1 may be a group represented by one of Formulae 6-1 to 6-171:

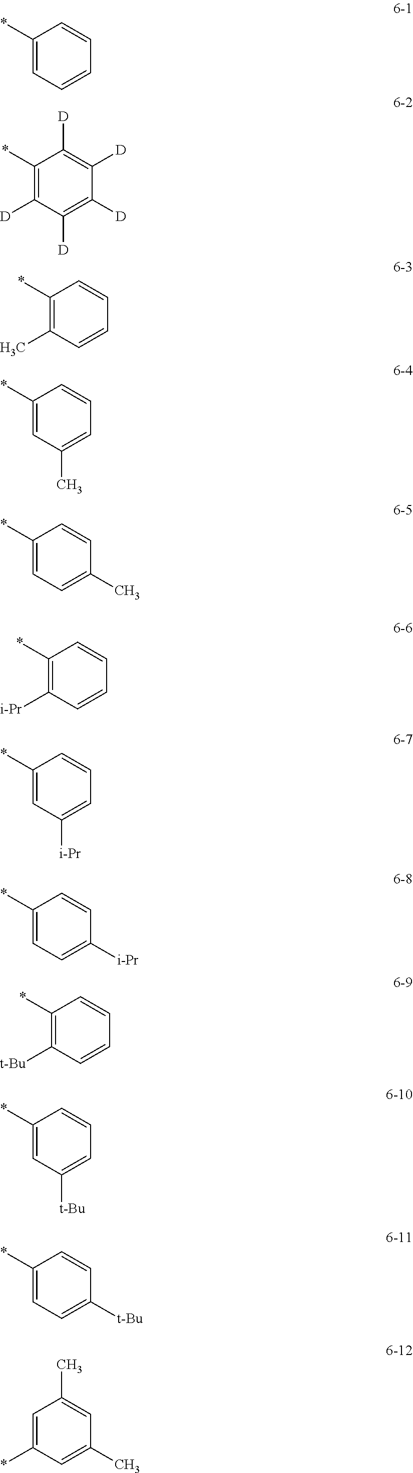

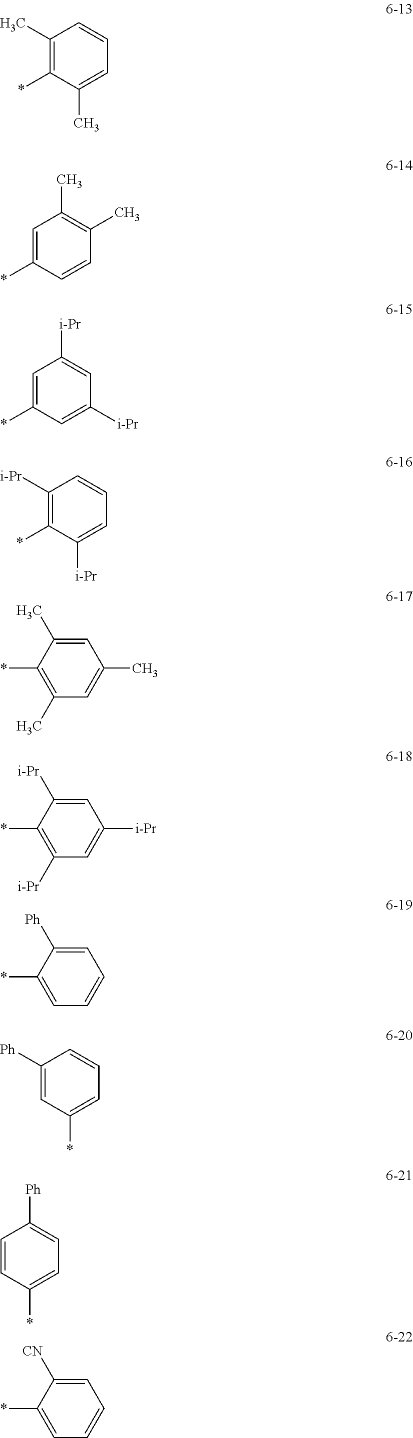

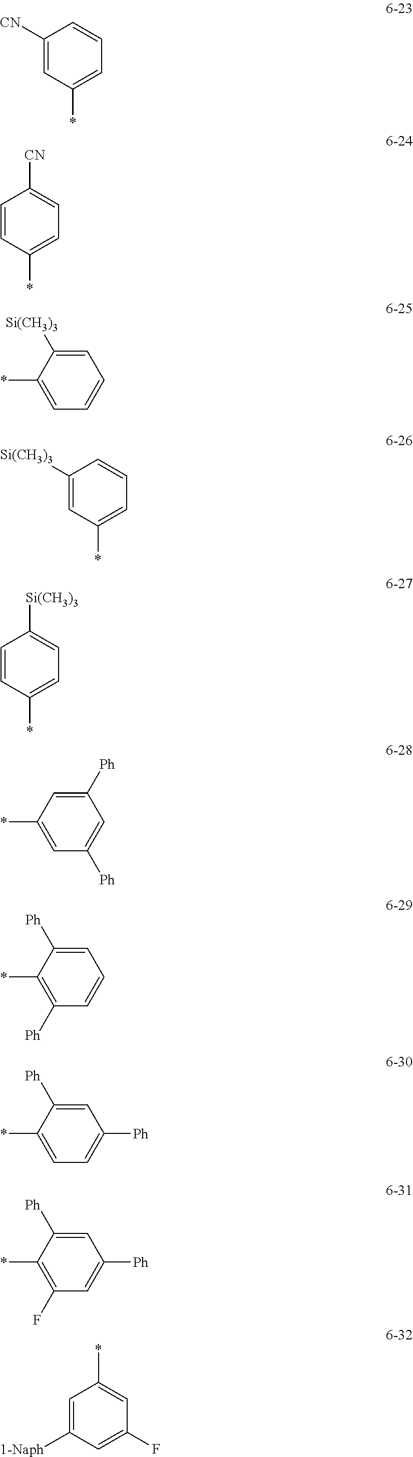

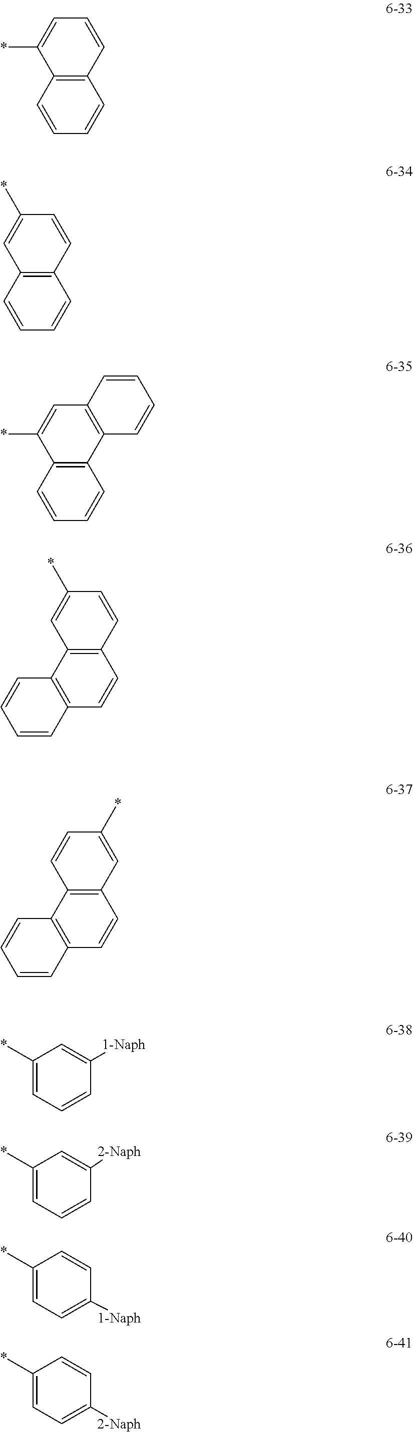

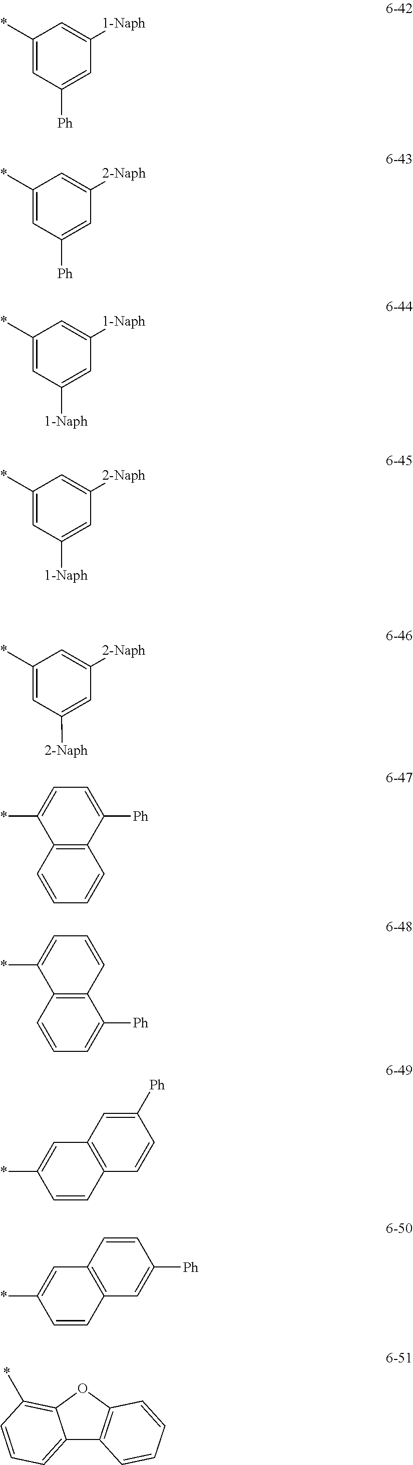

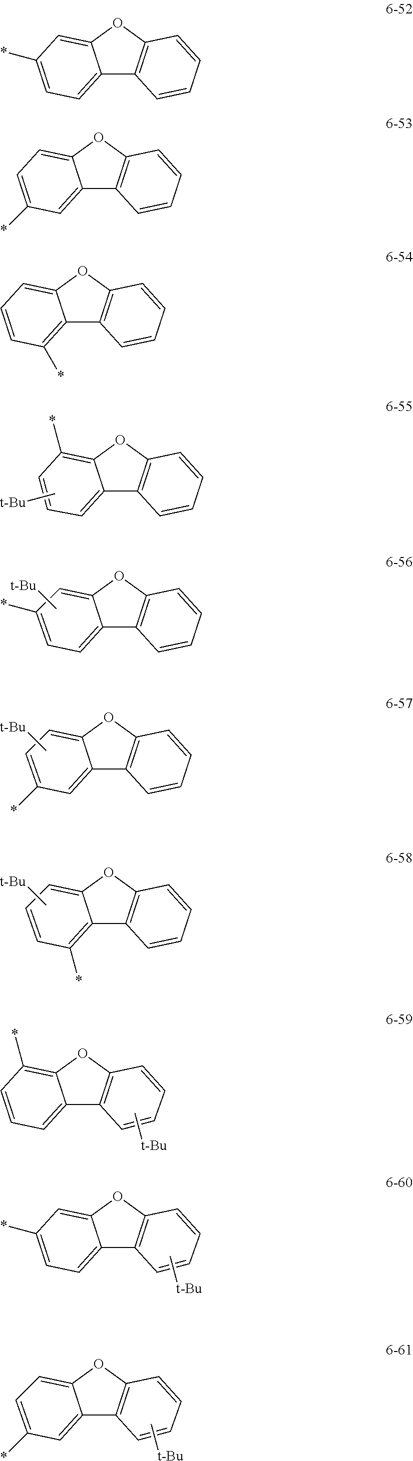

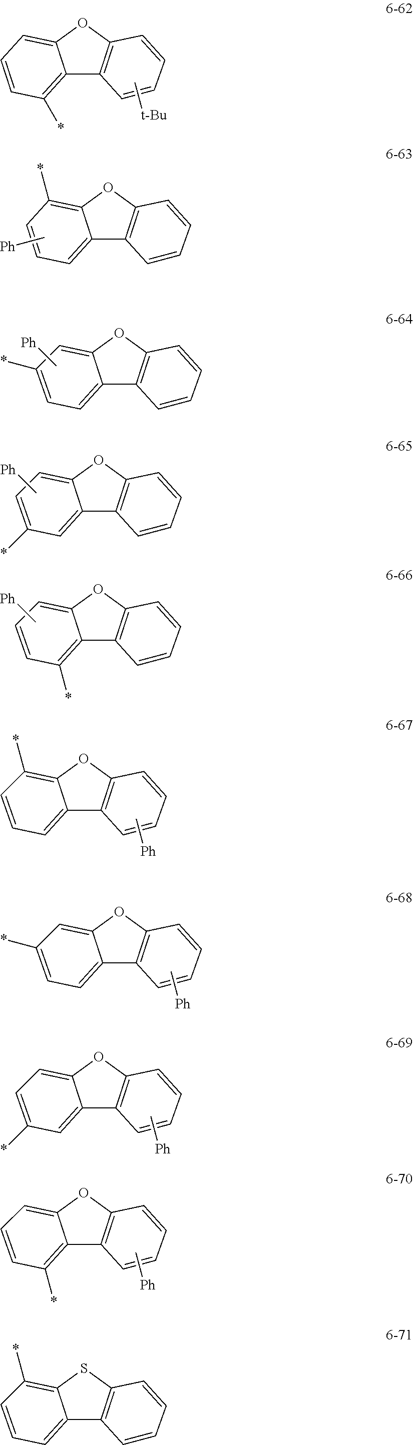

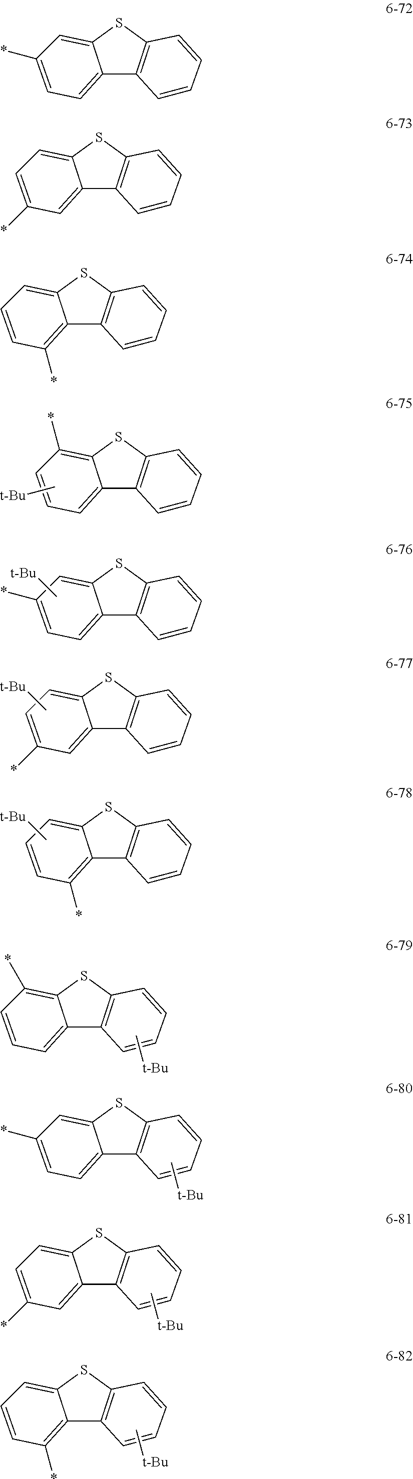

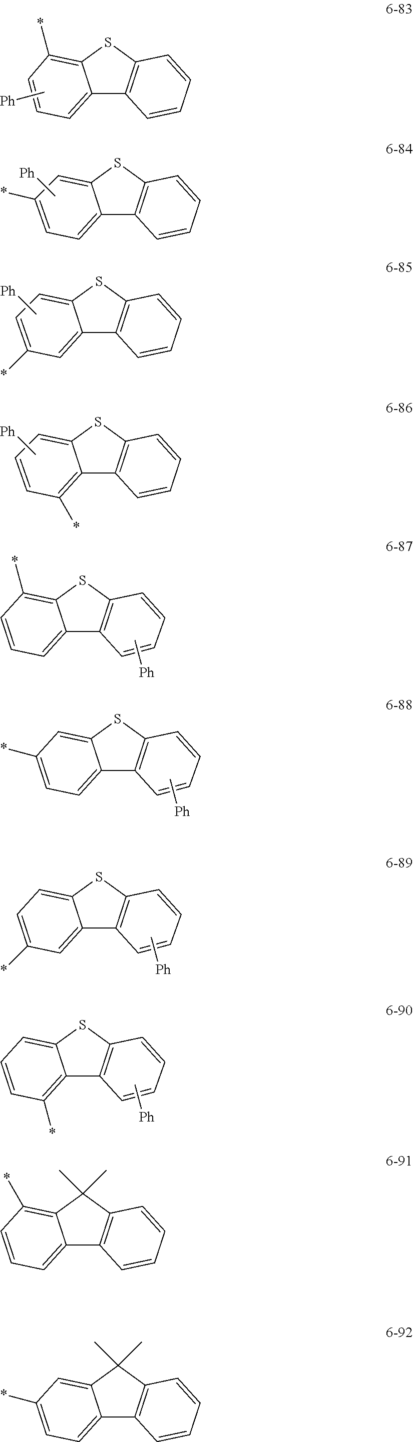

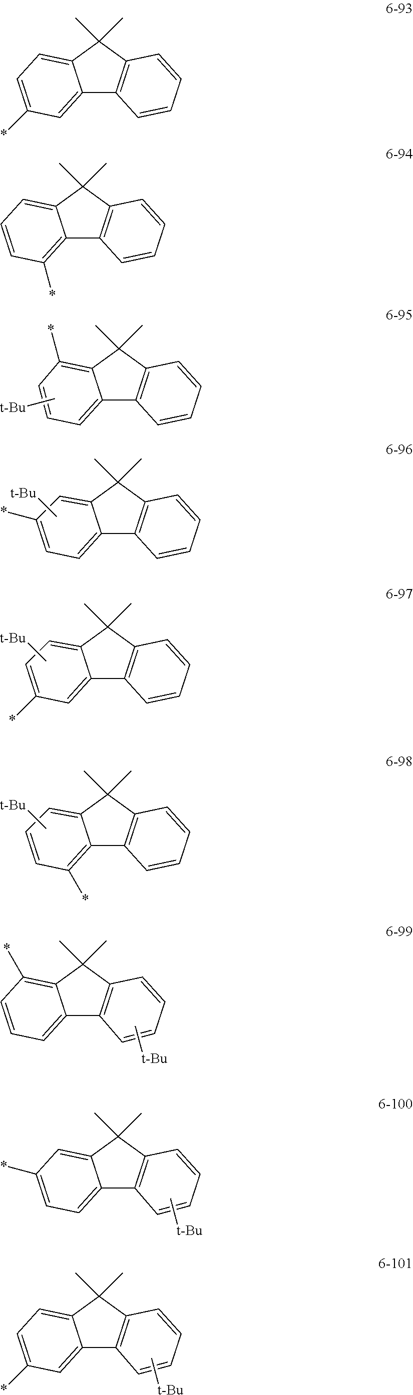

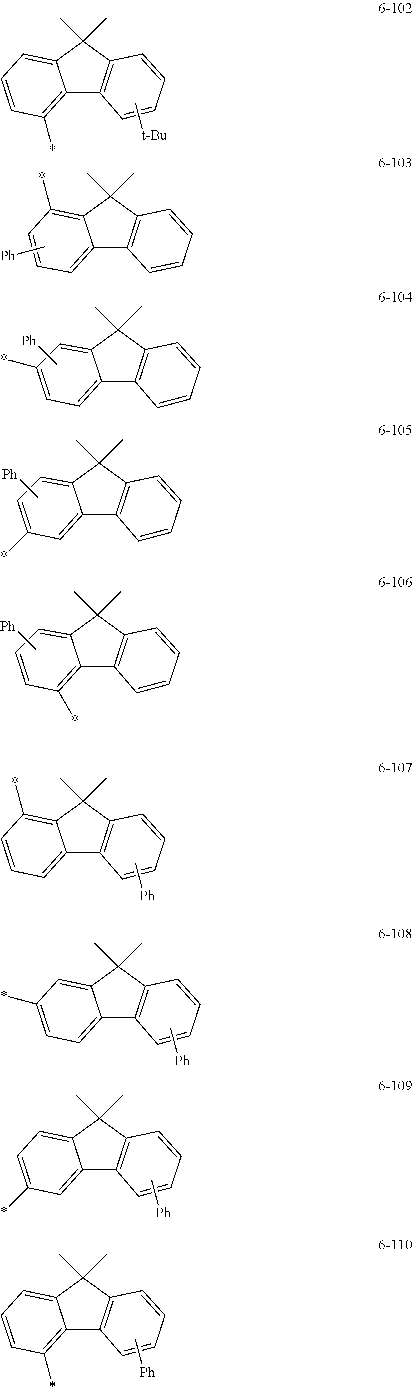

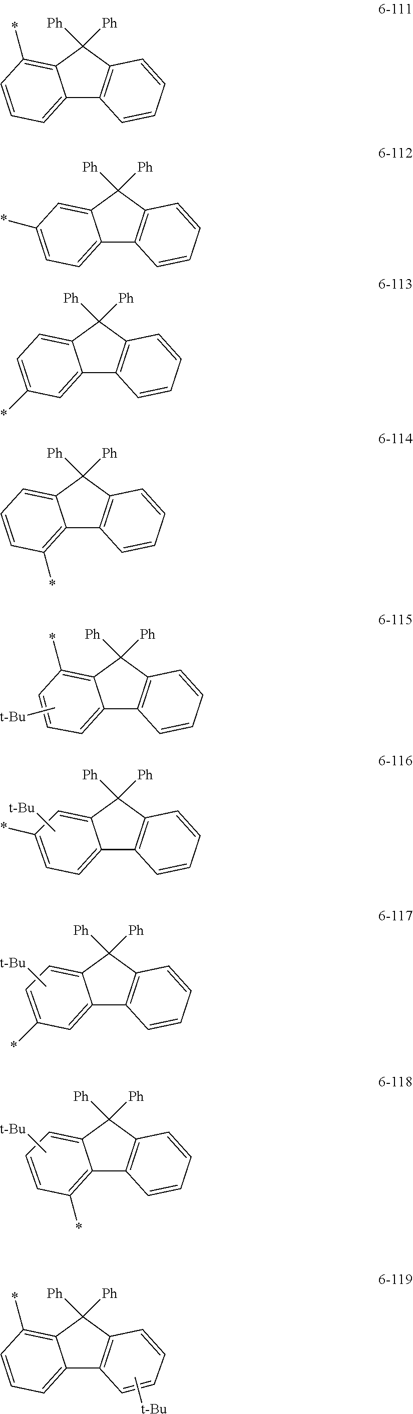

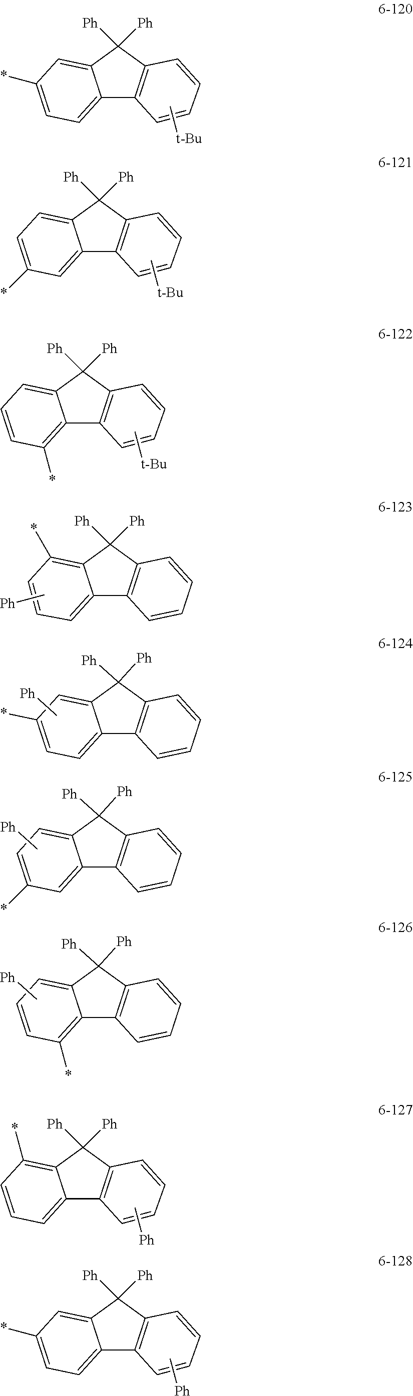

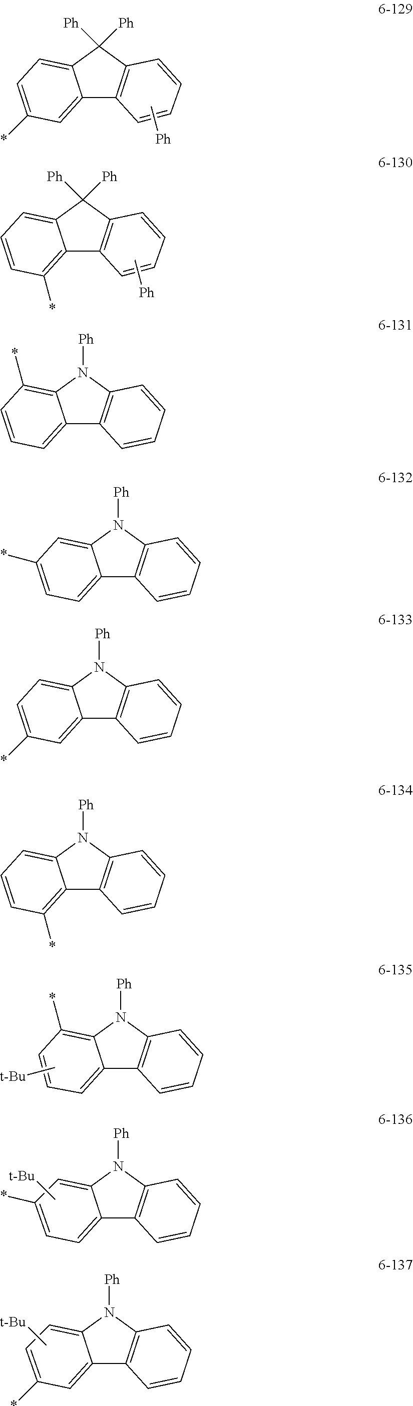

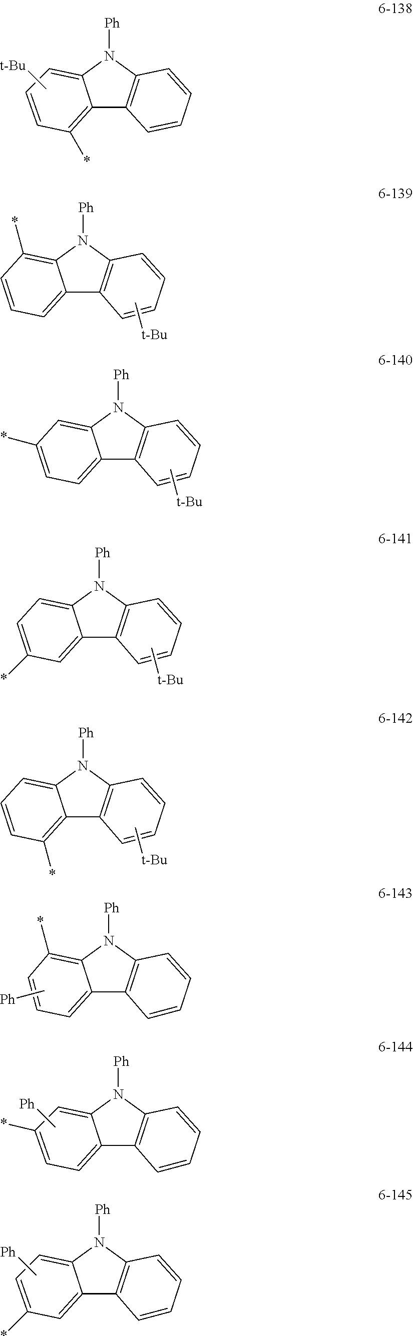

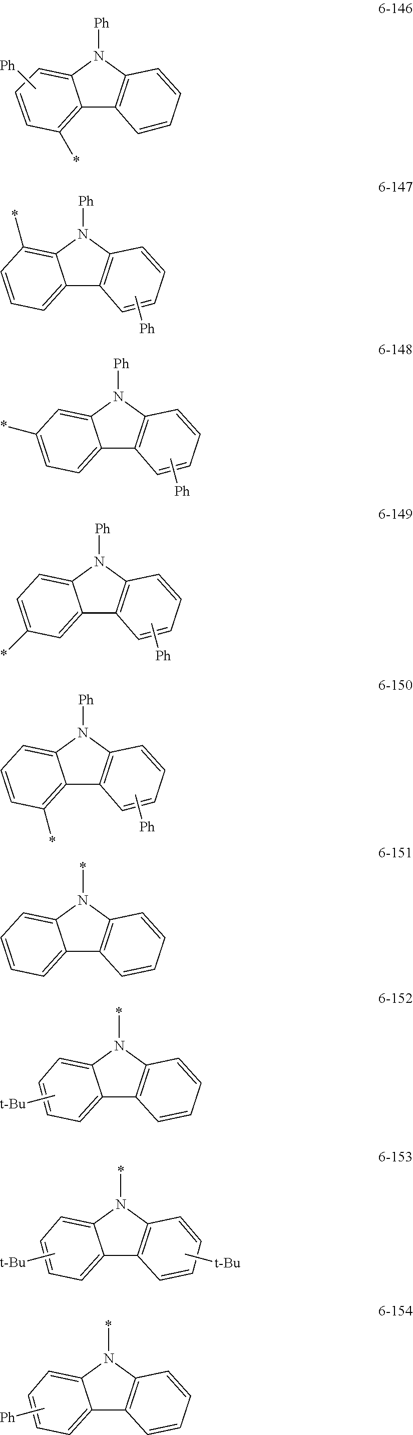

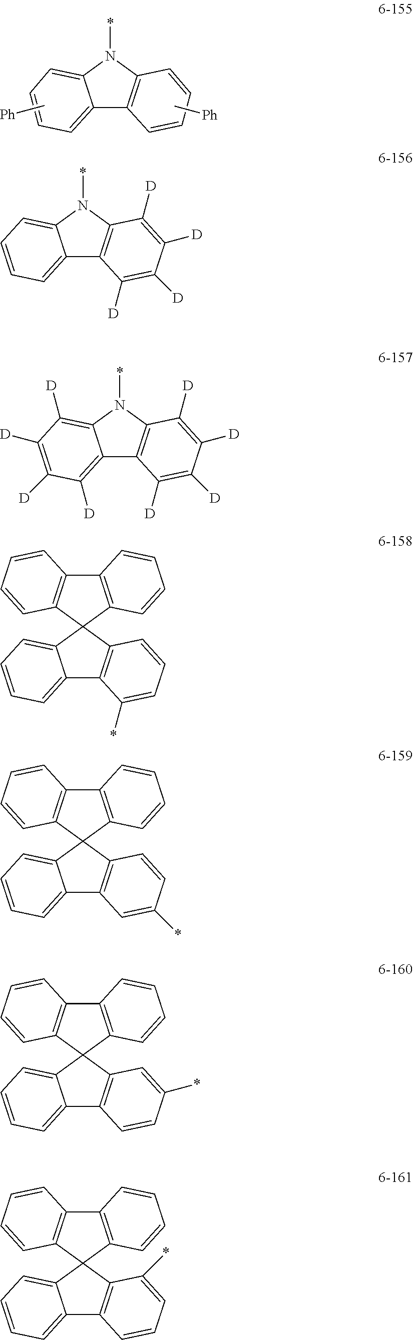

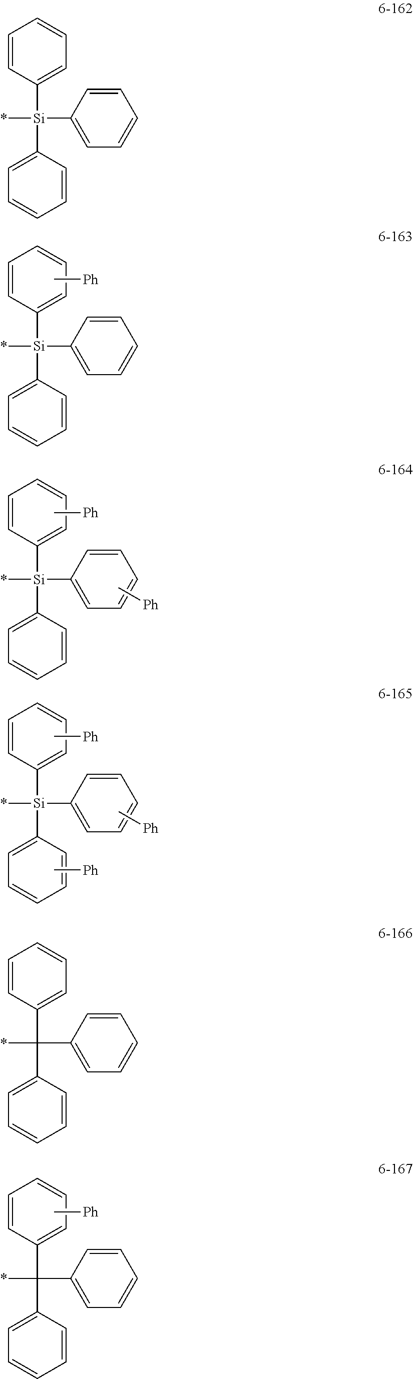

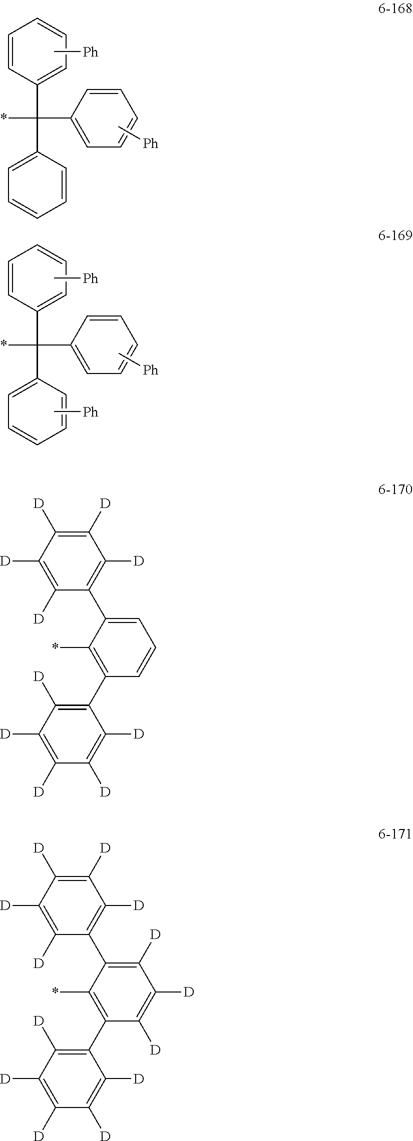

wherein, in Formulae 6-1 to 6-171,

i-Pr may be an isopropyl group;

t-Bu may be a tert-butyl group;

Ph may be a phenyl group;

1-Naph may be a 1-naphthyl group;

2-Naph may be a 2-naphthyl group; and

* indicates a binding site to an adjacent atom.

b21 in Formula 1 may be an integer selected from 1 to 5. When b21 is 2 or more, a plurality of R₂₁(s) may be identical to or different from each other.

In one or more embodiments, b21 in Formula 1 may be 1.

In one or more embodiments, R₁₁ to R₂₀ in Formula 1 may each independently be a group represented by *-(L₂₁)ₐ₂₁-(R₂₁)_{b21}, hydrogen, deuterium, —F, —Cl, —Br, —I, a cyano group, a C₁-C₂₀ alkyl group, a C₁-C₂₀ alkyl group substituted with at least one phenyl group, or a C₁-C₂₀ alkoxy group;

a cyclopentyl group, a cyclohexyl group, a cycloheptyl group, a cyclopentenyl group, a cyclohexenyl group, a phenyl group, a biphenyl group, a terphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, an indolyl group, an isoindolyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a dibenzofuranyl group, a dibenzothiophenyl group, a dibenzosilolyl group, a carbazolyl group, a benzocarbazolyl group, a dibenzocarbazolyl group, an azafluorenyl group, an azaspiro-bifluorenyl group, a dibenzothiophenylfluorenyl group, an azacarbazolyl group, a diazacarbazolyl group, an azadibenzofuranyl group, an azadibenzothiophenyl group, an azadibenzosilolyl group, an imidazopyridinyl group, or an imidazopyrimidinyl group, each unsubstituted or substituted with deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkyl group substituted with at least one phenyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, an indolyl group, an isoindolyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a dibenzofuranyl group, a dibenzothiophenyl group, a dibenzosilolyl group, a carbazolyl group, a benzocarbazolyl group, a dibenzocarbazolyl group, an azafluorenyl group, an azaspiro-bifluorenyl group, a dibenzothiophenylfluorenyl group, an azacarbazolyl group, a diazacarbazolyl group, an azadibenzofuranyl group, an azadibenzothiophenyl group, an azadibenzosilolyl group, an imidazopyridinyl group, an imidazopyrimidinyl group, —Si(Q₃₁)(Q₃₂)(Q₃₃), —C(Q₃₁)(Q₃₂)(Q₃₃), —N(Q₃₁)(Q₃₂), —B(Q₃₁)(Q₃₂), or any combination thereof; or

—Si(Q₁)(Q₂)(Q₃), —C(Q₁)(Q₂)(Q₃), —N(Q₁)(Q₂), or —B(Q₁)(Q₂),

wherein Q₁ to Q₃ and Q₃₁ to Q₃₃ may each independently be a C₁-C₁₀ alkyl group, a C₁-C₁₀ alkoxy group, a phenyl group, a phenyl group substituted with a cyano group, a biphenyl group, a terphenyl group, or a naphthyl group.

In one or more embodiments, R₁₁ to R₂₀ in Formula 1 may each independently be a group represented by *-(L₂₁)ₐ₂₁-(R₂₁)_{b21}, hydrogen, deuterium, —F, —Cl, —Br, —I, a cyano group, a C₁-C₂₀ alkyl group, or a group represented by one of Formulae 5-1 to 5-20.

In one or more embodiments, R₁₁ to R₂₀ in Formula 1 may each independently be a group represented by *-(L₂₁)ₐ₂₁-(R₂₁)_{b21}, hydrogen, deuterium, —F, —Cl, —Br, —I, a cyano group, a methyl group, an ethyl group, an n-propyl group, an isopropyl group, an n-butyl group, a sec-butyl group, an isobutyl group, a tert-butyl group, or a group represented by one of Formulae 6-1 to 6-171.

In one or more embodiments, one or two of R₁₁ to R₁₈ in Formula 1 may be a group represented by *-(L₂₁)ₐ₂₁-(R₂₁)_{b21}, and the others of R₁₁ to R₁₈, and R₁₉ and R₂₀ may each independently be hydrogen, deuterium, —F, —Cl, —Br, —I, a cyano group, a methyl group, an ethyl group, an n-propyl group, an isopropyl group, an n-butyl group, a sec-butyl group, an isobutyl group, a tert-butyl group, or a group represented by one of Formulae 6-1 to 6-171.

In one or more embodiments, one or two of R₁₁ to R₁₈ in Formula 1 may be a group represented by *-(L₂₁)ₐ₂₁-(R₂₁)_{b21}, and the others of R₁₁ to R₁₈, and R₁₉ and R₂₀ may each independently be hydrogen, deuterium, —F, —Cl, —Br, —I, a cyano group, a methyl group, an ethyl group, an n-propyl group, an isopropyl group, an n-butyl group, a sec-butyl group, an isobutyl group, a tert-butyl group, a phenyl group, a biphenyl group, a terphenyl group, or a carbazolyl group.

In one or more embodiments, one of R₁₁ to R₁₈ in Formula 1 may be a group represented by *-(L₂₁)ₐ₂₁-(R₂₁)_{b21}, and the others of R₁₁ to R₁₈, and R₁₉ and R₂₀ may each independently be hydrogen, deuterium, —F, —Cl, —Br, —I, a cyano group, a methyl group, an ethyl group, an n-propyl group, an isopropyl group, an n-butyl group, a sec-butyl group, an isobutyl group, a tert-butyl group, or a group represented by one of Formulae 6-1 to 6-171.

In one or more embodiments, one of R₁₁ to R₁₈ in Formula 1 may be a group represented by *-(L₂₁)ₐ₂₁-(R₂₁)_{b21}, and the others of R₁₁ to R₁₈, and R₁₉ and R₂₀ may each independently be hydrogen, deuterium, —F, —Cl, —Br, —I, a cyano group, a methyl group, an ethyl group, an n-propyl group, an isopropyl group, an n-butyl group, a sec-butyl group, an isobutyl group, a tert-butyl group, a phenyl group, a biphenyl group, a terphenyl group, or a carbazolyl group.

R₁₀ₐ in Formula 1 may be:

deuterium (-D), —F, —Cl, —Br, —I, a hydroxyl group, a cyano group, or a nitro group,

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —C(Q₁₁)(Q₁₂)(Q₁₃), —B(Q₁₁)(Q₁₂), —N(Q₁₁)(Q₁₂), —P(Q₁₁)(Q₁₂), —C(═O)(Q₁₁), —S(═O)(Q₁₁), —S(═O)₂(Q₁₁), —P(═O)(Q₁₁)(Q₁₂), —P(═S)(Q₁₁)(Q₁₂), or any combination thereof,

a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —C(Q₂₁)(Q₂₂)(Q₂₃), —B(Q₂₁)(Q₂₂), —N(Q₂₁)(Q₂₂), —P(Q₂₁)(Q₂₂), —C(═O)(Q₂₁), —S(═O)(Q₂₁), —S(═O)₂(Q₂₁), —P(═O)(Q₂₁)(Q₂₂) or —P(═S)(Q₂₁)(Q₂₂), or any combination thereof; or

—Si(Q₃₁)(Q₃₂)(Q₃₃), —C(Q₃₁)(Q₃₂)(Q₃₃), —B(Q₃₁)(Q₃₂), —N(Q₃₁)(Q₃₂), —P(Q₃₁)(Q₃₂), —C(═O)(Q₃₁), —S(═O)(Q₃₁), —S(═O)₂(Q₃₁), —P(═O)(Q₃₁)(Q₃₂), or —P(═S)(Q₃₁)(Q₃₂),

wherein Q₁ to Q₃, Q₁₁ to Q₁₃, Q₂₁ to Q₂₃ and Q₃₁ to Q₃₃ may each independently be: hydrogen; deuterium; —F; —Cl; —Br; —I; a hydroxyl group; a cyano group; a nitro group; a C₁-C₆ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and

* and *′ each indicate a binding site to a neighboring atom.

In one or more embodiments, the heterocyclic compound represented by Formula 1 may be represented by one of Formulae 1-1 to 1-8:

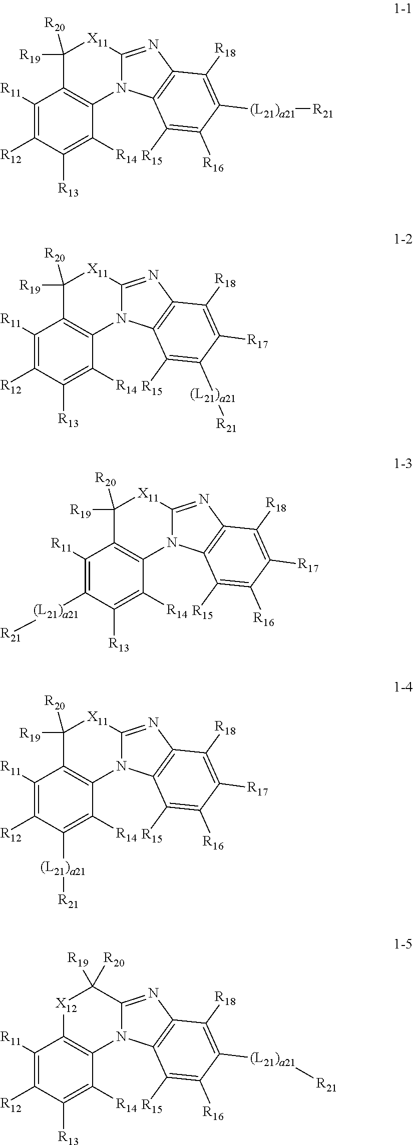

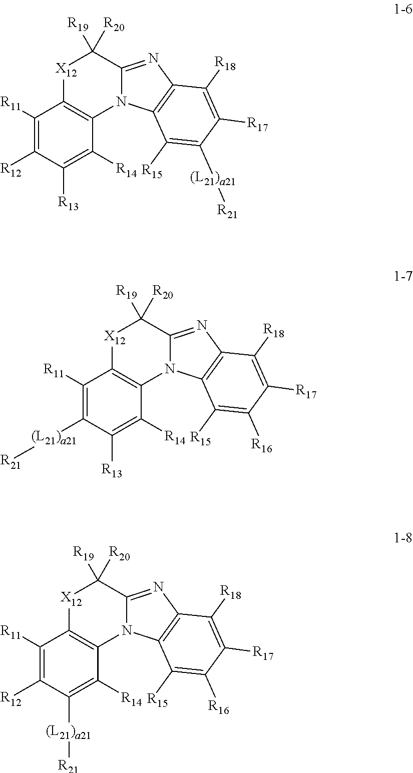

wherein, in Formulae 1-1 to 1-8,

X₁₁ and X₁₂ may each independently be O or S, and

L₂₁, a21, and R₁₁ to R₂₁ are the same as described in connection with Formula 1.

In one or more embodiments, L₂₁ in Formulae 1-1 to 1-8 may be a single bond, or *—Si(R₃₁)(R₃₂)—*′, or

a benzene group, a pentalene group, an indene group, a naphthalene group, an azulene group, a heptalene group, an indacene group, an acenaphthalene group, a fluorene group, a spiro-bifluorene group, a spiro-benzofluorene-fluorene group, a benzofluorene group, a dibenzofluorene group, a phenalene group, a phenanthrene group, an anthracene group, a fluoranthene group, a pyrene group, a chrysene group, a naphthacene group, a picene group, a perylene group, a pyrrole group, a thiophene group, a furan group, a silole group, an imidazole group, a pyrazole group, a thiazole group, an isothiazole group, an oxazole group, an isoxazole group, a pyridine group, a pyrazine group, a pyrimidine group, a pyridazine group, a triazine group, a benzofuran group, a benzothiophene group, a dibenzofuran group, a dibenzothiophene group, a carbazole group, a benzosilole group, a dibenzosilole group, a quinoline group, an isoquinoline group, a benzimidazole group, an imidazopyridine group, or an imidazopyrimidine group, each unsubstituted or substituted with deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a spiro-benzofluorene-fluorenyl group, a benzofluorenyl group, a dibenzofluorenyl group, a phenalenyl group, a phenanthrenyl group, an anthracenyl group, a fluoranthenyl group, a pyrenyl group, a chrysenyl group, a naphthacenyl group, a picenyl group, a perylenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, a benzofuranyl group, a benzothiophenyl group, a dibenzofuranyl group, a dibenzothiophenyl group, a carbazolyl group, a benzosilolyl group, a dibenzosilolyl group, a quinolinyl group, an isoquinolinyl group, a benzimidazolyl group, an imidazopyridinyl group, an imidazopyrimidinyl group, —Si(Q₃₁)(Q₃₂)(Q₃₃), —C(Q₃₁)(Q₃₂)(Q₃₃), —B(Q₃₁)(Q₃₂), —N(Q₃₁)(Q₃₂), —P(Q₃₁)(Q₃₂), —C(═O)(Q₃₁), —S(═O)(Q₃₁), —S(═O)₂(Q₃₁), —P(═O)(Q₃₁)(Q₃₂), —P(═S)(Q₃₁)(Q₃₂), or any combination thereof,

R₃₁ and R₃₂ may each independently be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a spiro-benzofluorene-fluorenyl group, a benzofluorenyl group, a dibenzofluorenyl group, a phenalenyl group, a phenanthrenyl group, an anthracenyl group, a fluoranthenyl group, a pyrenyl group, a chrysenyl group, a naphthacenyl group, a picenyl group, a perylenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, a benzofuranyl group, a benzothiophenyl group, a dibenzofuranyl group, a dibenzothiophenyl group, a carbazolyl group, a benzosilolyl group, a dibenzosilolyl group, a quinolinyl group, an isoquinolinyl group, a benzimidazolyl group, an imidazopyridinyl group, an imidazopyrimidinyl group, —Si(Q₃₁)(Q₃₂)(Q₃₃), —C(Q₃₁)(Q₃₂)(Q₃₃), —B(Q₃₁)(Q₃₂), —N(Q₃₁)(Q₃₂), —P(Q₃₁)(Q₃₂), —C(═O)(Q₃₁), —S(═O)(Q₃₁), —S(═O)₂(Q₃₁), —P(═O)(Q₃₁)(Q₃₂), or —P(═S)(Q₃₁)(Q₃₂), and

a21 may be 1, 2, or 3;

R₂₁ may be a cyclopentenyl group, a cyclohexenyl group, a phenyl group, a biphenyl group, a terphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, an indolyl group, an isoindolyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a dibenzofuranyl group, a dibenzothiophenyl group, a dibenzosilolyl group, a carbazolyl group, a benzocarbazolyl group, a dibenzocarbazolyl group, an azafluorenyl group, an azaspiro-bifluorenyl group, a dibenzothiophenylfluorenyl group, an azacarbazolyl group, a diazacarbazolyl group, an azadibenzofuranyl group, an azadibenzothiophenyl group, an azadibenzosilolyl group, an imidazopyridinyl group, or an imidazopyrimidinyl group, each unsubstituted or substituted with deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkyl group substituted with at least one phenyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, an indolyl group, an isoindolyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a dibenzofuranyl group, a dibenzothiophenyl group, a dibenzosilolyl group, a carbazolyl group, a benzocarbazolyl group, a dibenzocarbazolyl group, an azafluorenyl group, an azaspiro-bifluorenyl group, a dibenzothiophenylfluorenyl group, an azacarbazolyl group, a diazacarbazolyl group, an azadibenzofuranyl group, an azadibenzothiophenyl group, an azadibenzosilolyl group, an imidazopyridinyl group, an imidazopyrimidinyl group, —Si(Q₃₁)(Q₃₂)(Q₃₃), —C(Q₃₁)(Q₃₂)(Q₃₃), —N(Q₃₁)(Q₃₂), —B(Q₃₁)(Q₃₂), or any combination thereof; or

—Si(Q₁)(Q₂)(Q₃), —C(Q₁)(Q₂)(Q₃), —N(Q₁)(Q₂), or —B(Q₁)(Q₂),

Q₁ to Q₃ and Q₃₁ to Q₃₃ may each independently be a C₁-C₁₀ alkyl group, a C₁-C₁₀ alkoxy group, a phenyl group, a phenyl group substituted with a cyano group, a biphenyl group, a terphenyl group, or a naphthyl group, and

R₁₁ to R₂₀ may each independently be hydrogen, deuterium, —F, —Cl, —Br, —I, a cyano group, a methyl group, an ethyl group, an n-propyl group, an isopropyl group, an n-butyl group, a sec-butyl group, an isobutyl group, a tert-butyl group, a phenyl group, a biphenyl group, a terphenyl group, or a carbazolyl group.

In one or more embodiments, the heterocyclic compound represented by Formula 1 may be selected from Group I and Group II:

Group I

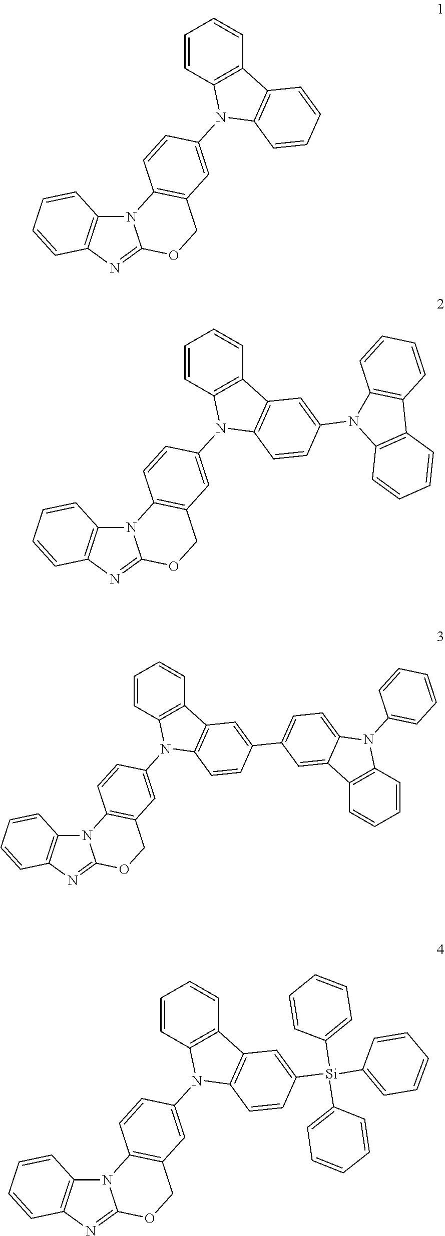 [SMILES: c1ccc2c(c1)nc1n2-c2ccc(-n3c4ccccc4c4ccccc43)cc2CO1 ; c1ccc2c(c1)nc1n2-c2ccc(-n3c4ccccc4c4cc(-n5c6ccccc6c6ccccc65)ccc43)cc2CO1 ; c1ccc(-n2c3ccccc3c3cc(-c4ccc5c(c4)c4ccccc4n5-c4ccc5c(c4)COc4nc6ccccc6n4-5)ccc32)cc1 ; c1ccc([Si](c2ccccc2)(c2ccccc2)c2ccc3c(c2)c2ccccc2n3-c2ccc3c(c2)COc2nc4ccccc4n2-3)cc1]

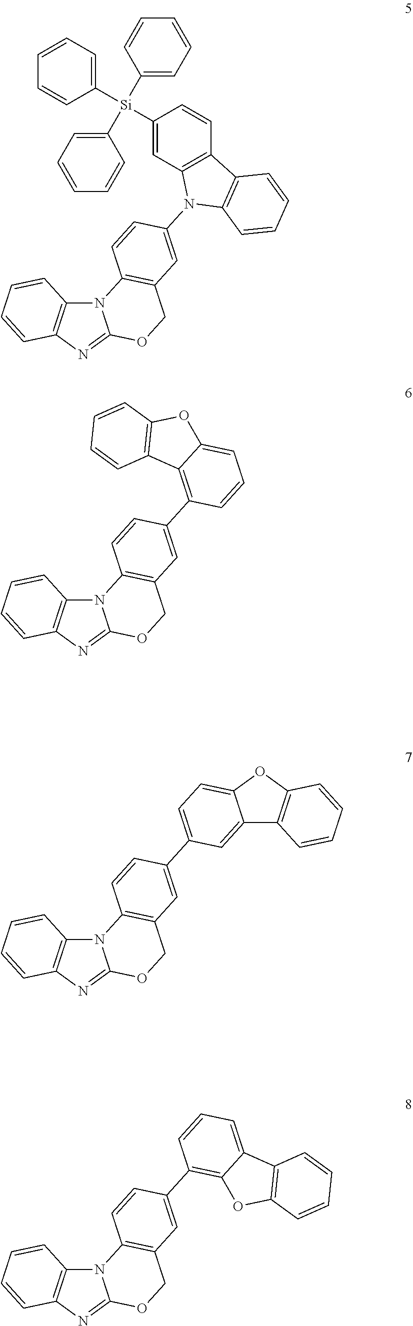 [SMILES: c1ccc([Si](c2ccccc2)(c2ccccc2)c2ccc3c4ccccc4n(-c4ccc5c(c4)COc4nc6ccccc6n4-5)c3c2)cc1 ; c1ccc2c(c1)nc1n2-c2ccc(-c3cccc4oc5ccccc5c34)cc2CO1 ; c1ccc2c(c1)nc1n2-c2ccc(-c3ccc4oc5ccccc5c4c3)cc2CO1 ; c1ccc2c(c1)nc1n2-c2ccc(-c3cccc4c3oc3ccccc34)cc2CO1]

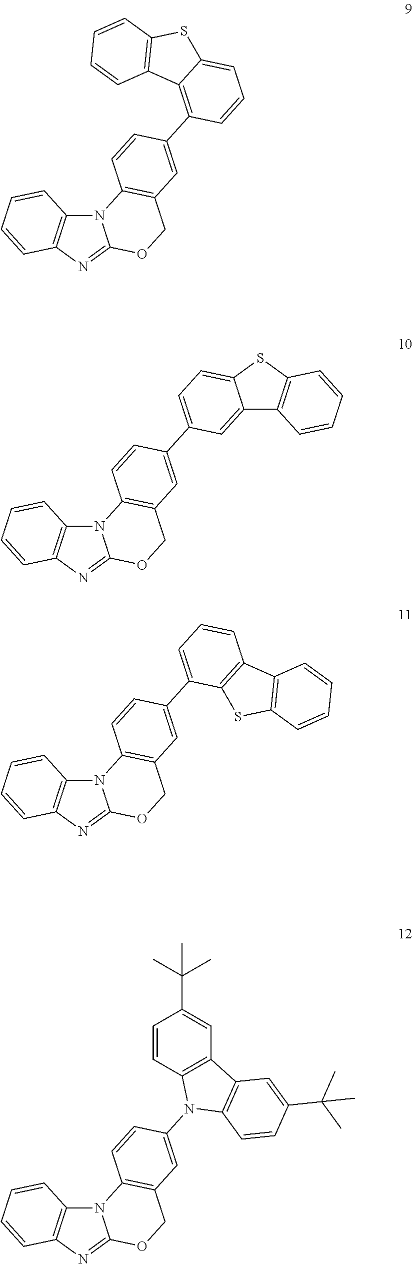 [SMILES: c1ccc2c(c1)nc1n2-c2ccc(-c3cccc4sc5ccccc5c34)cc2CO1 ; c1ccc2c(c1)nc1n2-c2ccc(-c3ccc4sc5ccccc5c4c3)cc2CO1 ; c1ccc2c(c1)nc1n2-c2ccc(-c3cccc4c3sc3ccccc34)cc2CO1 ; CC(C)(C)c1ccc2c(c1)c1cc(C(C)(C)C)ccc1n2-c1ccc2c(c1)COc1nc3ccccc3n1-2]

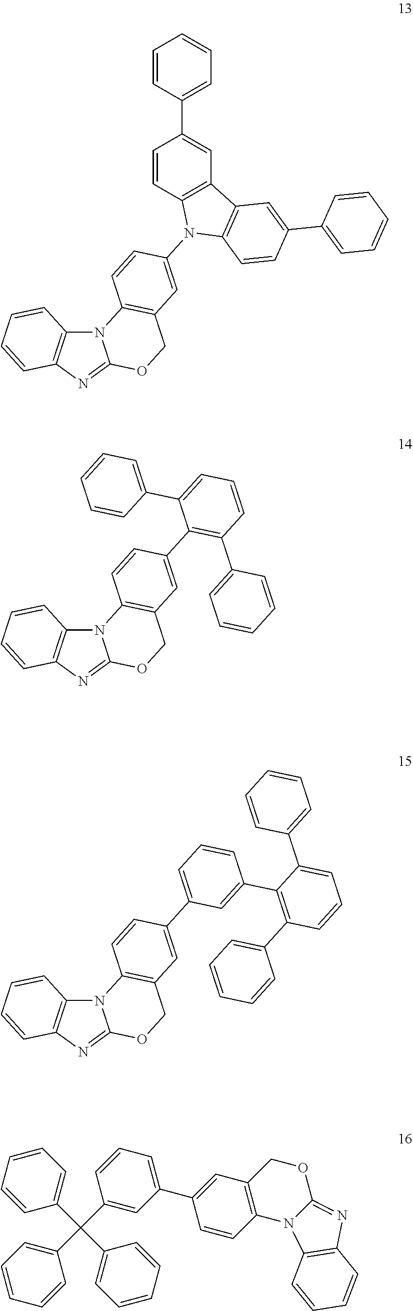 [SMILES: c1ccc(-c2ccc3c(c2)c2cc(-c4ccccc4)ccc2n3-c2ccc3c(c2)COc2nc4ccccc4n2-3)cc1 ; c1ccc(-c2cccc(-c3ccccc3)c2-c2ccc3c(c2)COc2nc4ccccc4n2-3)cc1 ; c1ccc(-c2cccc(-c3ccccc3)c2-c2cccc(-c3ccc4c(c3)COc3nc5ccccc5n3-4)c2)cc1 ; c1ccc(C(c2ccccc2)(c2ccccc2)c2cccc(-c3ccc4c(c3)COc3nc5ccccc5n3-4)c2)cc1]

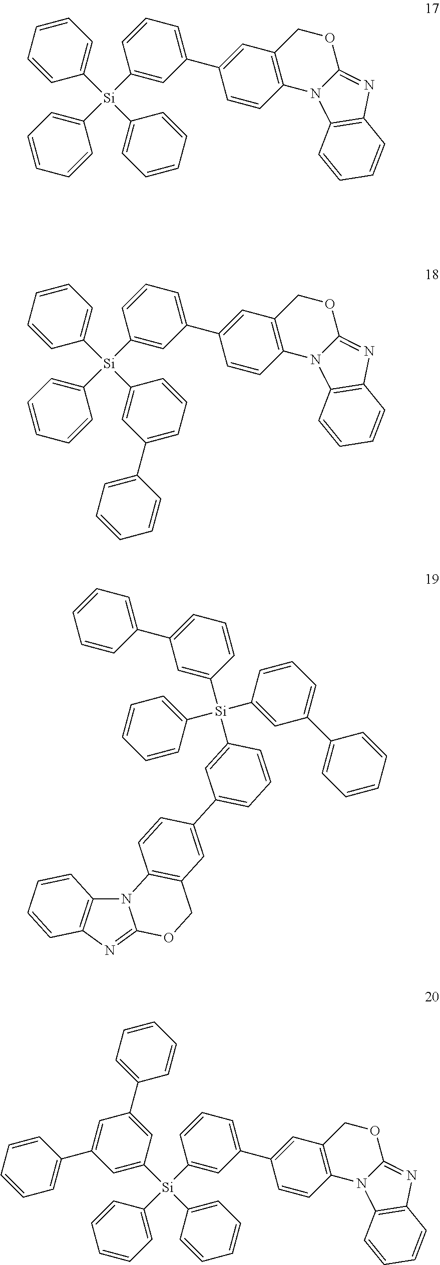 [SMILES: c1ccc([Si](c2ccccc2)(c2ccccc2)c2cccc(-c3ccc4c(c3)COc3nc5ccccc5n3-4)c2)cc1 ; c1ccc(-c2cccc([Si](c3ccccc3)(c3ccccc3)c3cccc(-c4ccc5c(c4)COc4nc6ccccc6n4-5)c3)c2)cc1 ; c1ccc(-c2cccc([Si](c3ccccc3)(c3cccc(-c4ccccc4)c3)c3cccc(-c4ccc5c(c4)COc4nc6ccccc6n4-5)c3)c2)cc1 ; c1ccc(-c2cc(-c3ccccc3)cc([Si](c3ccccc3)(c3ccccc3)c3cccc(-c4ccc5c(c4)COc4nc6ccccc6n4-5)c3)c2)cc1]

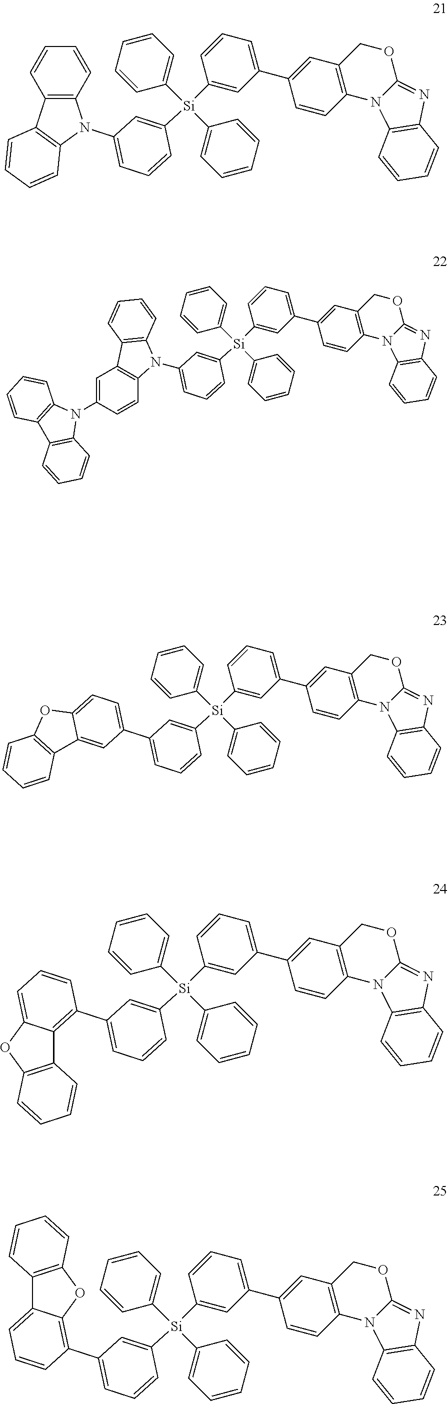

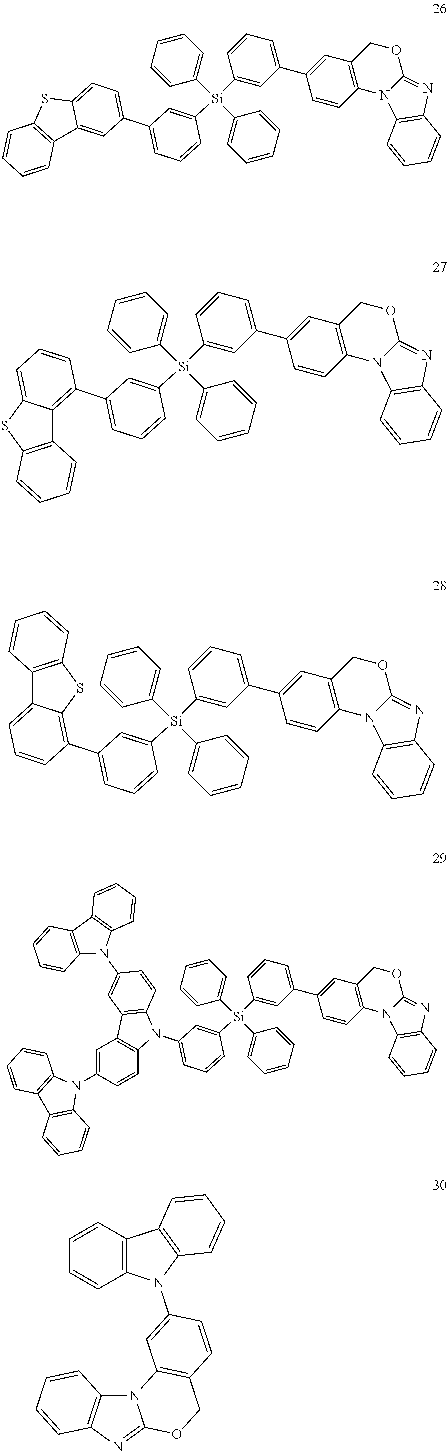 [SMILES: c1ccc([Si](c2ccccc2)(c2cccc(-c3ccc4c(c3)COc3nc5ccccc5n3-4)c2)c2cccc(-c3ccc4sc5ccccc5c4c3)c2)cc1 ; c1ccc([Si](c2ccccc2)(c2cccc(-c3ccc4c(c3)COc3nc5ccccc5n3-4)c2)c2cccc(-c3cccc4sc5ccccc5c34)c2)cc1 ; c1ccc([Si](c2ccccc2)(c2cccc(-c3ccc4c(c3)COc3nc5ccccc5n3-4)c2)c2cccc(-c3cccc4c3sc3ccccc34)c2)cc1 ; c1ccc([Si](c2ccccc2)(c2cccc(-c3ccc4c(c3)COc3nc5ccccc5n3-4)c2)c2cccc(-n3c4ccc(-n5c6ccccc6c6ccccc65)cc4c4cc(-n5c6ccccc6c6ccccc65)ccc43)c2)cc1 ; c1ccc2c(c1)nc1n2-c2cc(-n3c4ccccc4c4ccccc43)ccc2CO1]

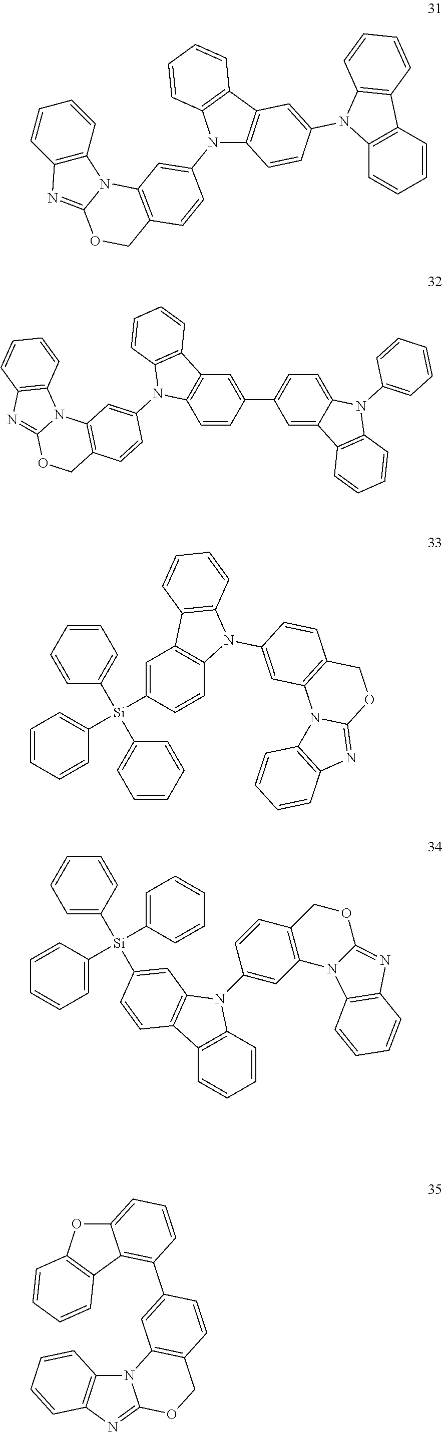 [SMILES: c1ccc2c(c1)nc1n2-c2cc(-n3c4ccccc4c4cc(-n5c6ccccc6c6ccccc65)ccc43)ccc2CO1 ; c1ccc(-n2c3ccccc3c3cc(-c4ccc5c(c4)c4ccccc4n5-c4ccc5c(c4)-n4c(nc6ccccc64)OC5)ccc32)cc1 ; c1ccc([Si](c2ccccc2)(c2ccccc2)c2ccc3c(c2)c2ccccc2n3-c2ccc3c(c2)-n2c(nc4ccccc42)OC3)cc1 ; c1ccc([Si](c2ccccc2)(c2ccccc2)c2ccc3c4ccccc4n(-c4ccc5c(c4)-n4c(nc6ccccc64)OC5)c3c2)cc1 ; c1ccc2c(c1)nc1n2-c2cc(-c3cccc4oc5ccccc5c34)ccc2CO1]

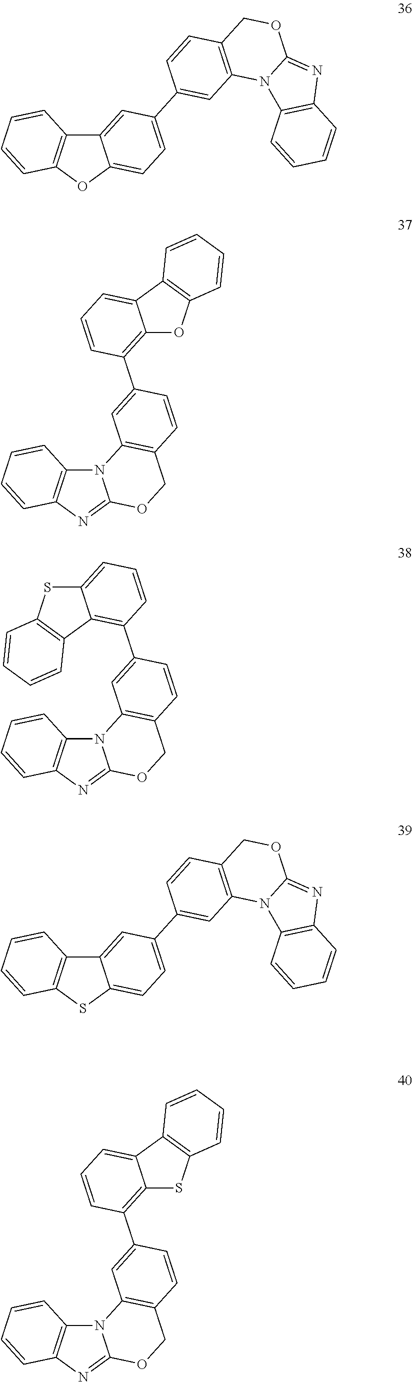

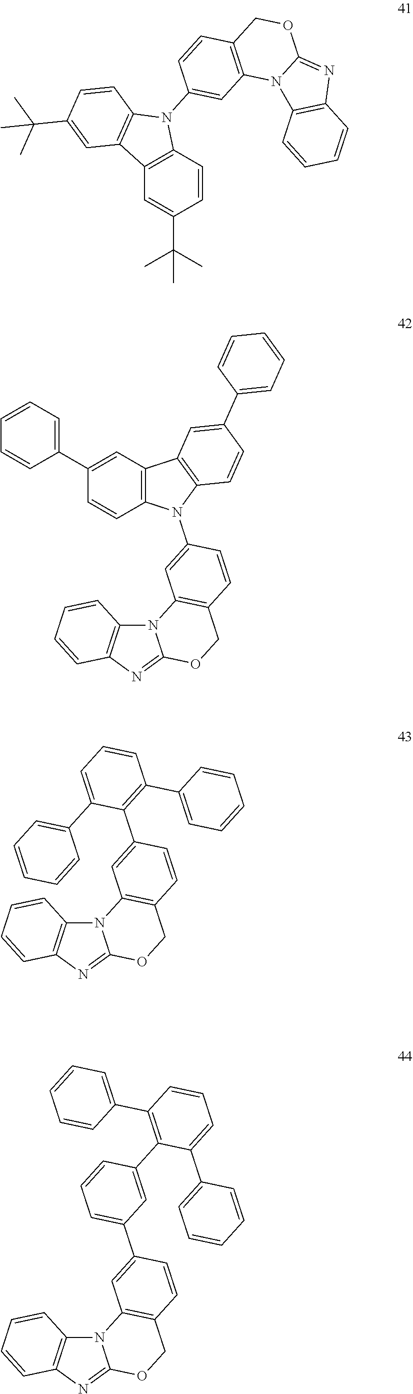

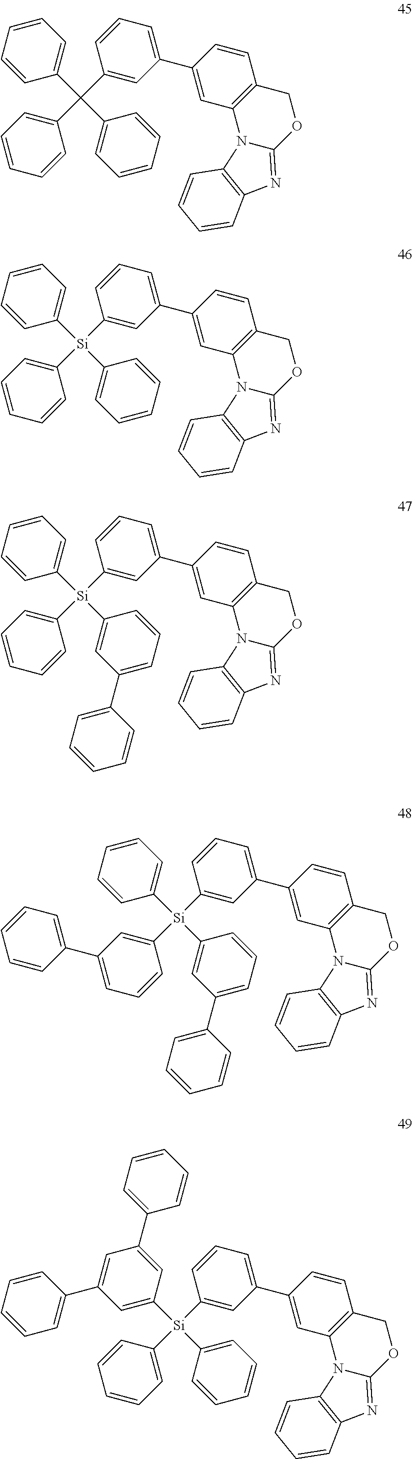 [SMILES: c1ccc(C(c2ccccc2)(c2ccccc2)c2cccc(-c3ccc4c(c3)-n3c(nc5ccccc53)OC4)c2)cc1 ; c1ccc([Si](c2ccccc2)(c2ccccc2)c2cccc(-c3ccc4c(c3)-n3c(nc5ccccc53)OC4)c2)cc1 ; c1ccc(-c2cccc([Si](c3ccccc3)(c3ccccc3)c3cccc(-c4ccc5c(c4)-n4c(nc6ccccc64)OC5)c3)c2)cc1 ; c1ccc(-c2cccc([Si](c3ccccc3)(c3cccc(-c4ccccc4)c3)c3cccc(-c4ccc5c(c4)-n4c(nc6ccccc64)OC5)c3)c2)cc1 ; c1ccc(-c2cc(-c3ccccc3)cc([Si](c3ccccc3)(c3ccccc3)c3cccc(-c4ccc5c(c4)-n4c(nc6ccccc64)OC5)c3)c2)cc1]

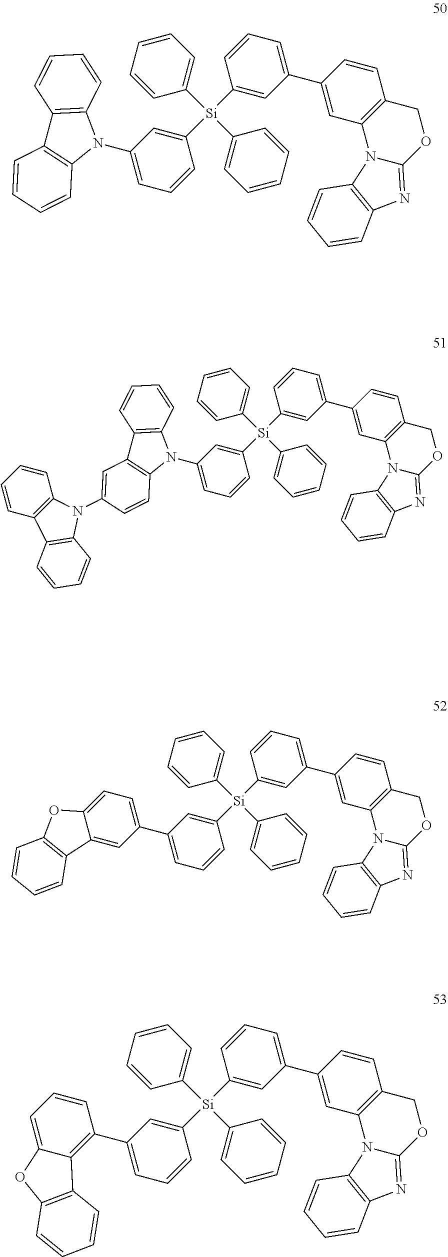 [SMILES: c1ccc([Si](c2ccccc2)(c2cccc(-c3ccc4c(c3)-n3c(nc5ccccc53)OC4)c2)c2cccc(-n3c4ccccc4c4ccccc43)c2)cc1 ; c1ccc([Si](c2ccccc2)(c2cccc(-c3ccc4c(c3)-n3c(nc5ccccc53)OC4)c2)c2cccc(-n3c4ccccc4c4cc(-n5c6ccccc6c6ccccc65)ccc43)c2)cc1 ; c1ccc([Si](c2ccccc2)(c2cccc(-c3ccc4c(c3)-n3c(nc5ccccc53)OC4)c2)c2cccc(-c3ccc4oc5ccccc5c4c3)c2)cc1 ; c1ccc([Si](c2ccccc2)(c2cccc(-c3ccc4c(c3)-n3c(nc5ccccc53)OC4)c2)c2cccc(-c3cccc4oc5ccccc5c34)c2)cc1]

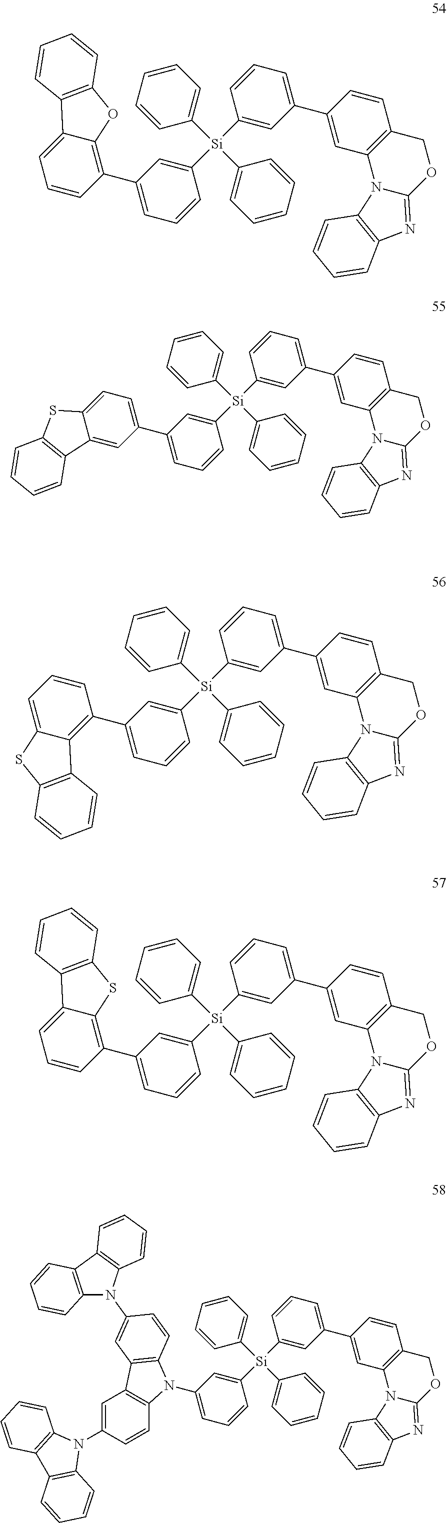

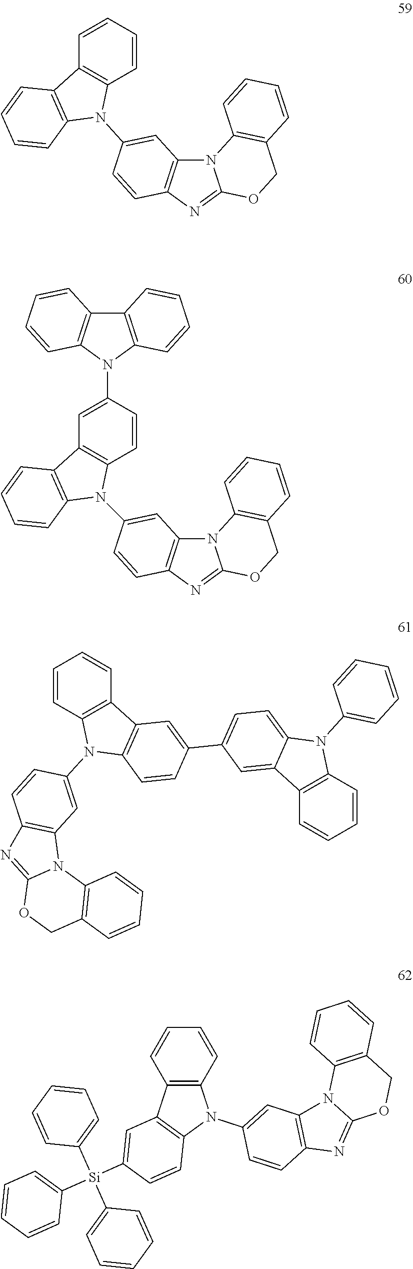 [SMILES: c1ccc2c(c1)COc1nc3ccc(-n4c5ccccc5c5ccccc54)cc3n1-2 ; c1ccc2c(c1)COc1nc3ccc(-n4c5ccccc5c5cc(-n6c7ccccc7c7ccccc76)ccc54)cc3n1-2 ; c1ccc(-n2c3ccccc3c3cc(-c4ccc5c(c4)c4ccccc4n5-c4ccc5nc6n(c5c4)-c4ccccc4CO6)ccc32)cc1 ; c1ccc([Si](c2ccccc2)(c2ccccc2)c2ccc3c(c2)c2ccccc2n3-c2ccc3nc4n(c3c2)-c2ccccc2CO4)cc1]

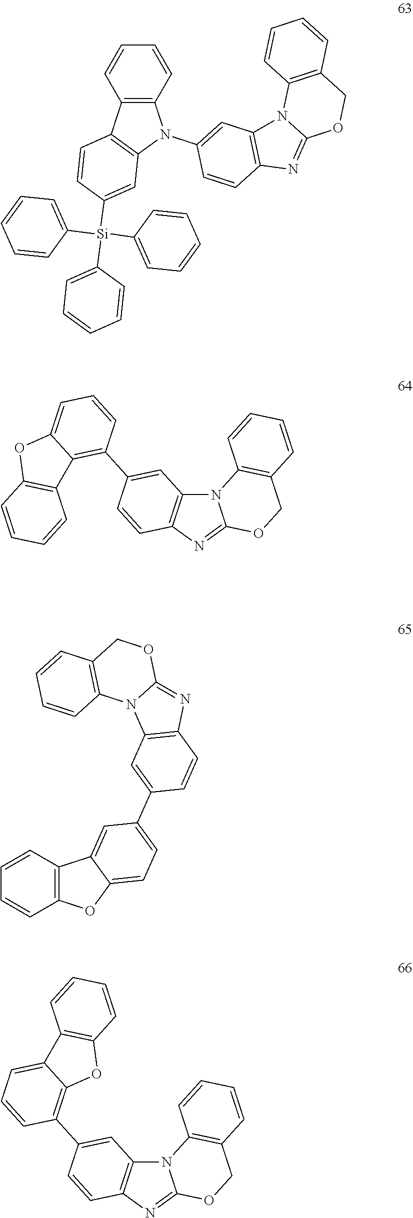 [SMILES: c1ccc([Si](c2ccccc2)(c2ccccc2)c2ccc3c4ccccc4n(-c4ccc5nc6n(c5c4)-c4ccccc4CO6)c3c2)cc1 ; c1ccc2c(c1)COc1nc3ccc(-c4cccc5oc6ccccc6c45)cc3n1-2 ; c1ccc2c(c1)COc1nc3ccc(-c4ccc5oc6ccccc6c5c4)cc3n1-2 ; c1ccc2c(c1)COc1nc3ccc(-c4cccc5c4oc4ccccc45)cc3n1-2]

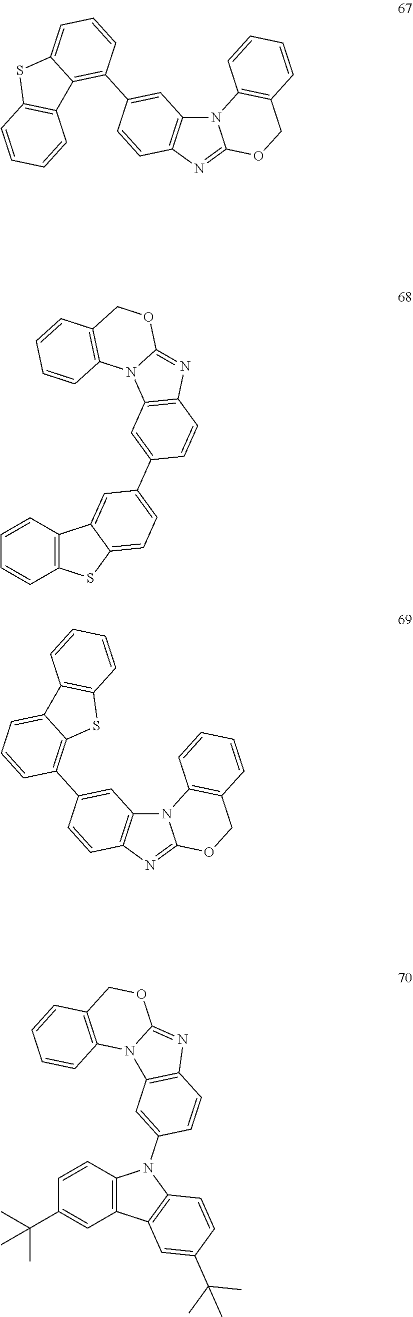 [SMILES: c1ccc2c(c1)COc1nc3ccc(-c4cccc5sc6ccccc6c45)cc3n1-2 ; c1ccc2c(c1)COc1nc3ccc(-c4ccc5sc6ccccc6c5c4)cc3n1-2 ; c1ccc2c(c1)COc1nc3ccc(-c4cccc5c4sc4ccccc45)cc3n1-2 ; CC(C)(C)c1ccc2c(c1)c1cc(C(C)(C)C)ccc1n2-c1ccc2nc3n(c2c1)-c1ccccc1CO3]

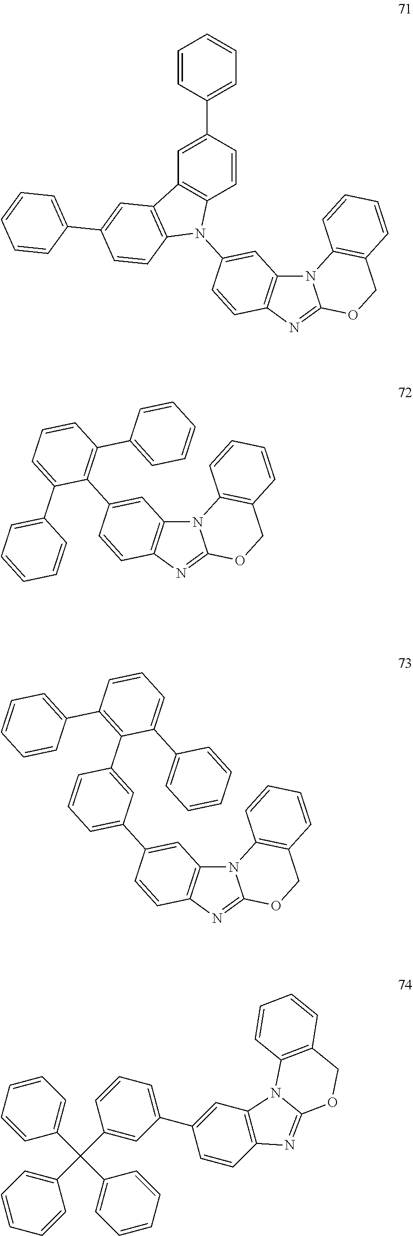 [SMILES: c1ccc(-c2ccc3c(c2)c2cc(-c4ccccc4)ccc2n3-c2ccc3nc4n(c3c2)-c2ccccc2CO4)cc1 ; c1ccc(-c2cccc(-c3ccccc3)c2-c2ccc3nc4n(c3c2)-c2ccccc2CO4)cc1 ; c1ccc(-c2cccc(-c3ccccc3)c2-c2cccc(-c3ccc4nc5n(c4c3)-c3ccccc3CO5)c2)cc1 ; c1ccc(C(c2ccccc2)(c2ccccc2)c2cccc(-c3ccc4nc5n(c4c3)-c3ccccc3CO5)c2)cc1]

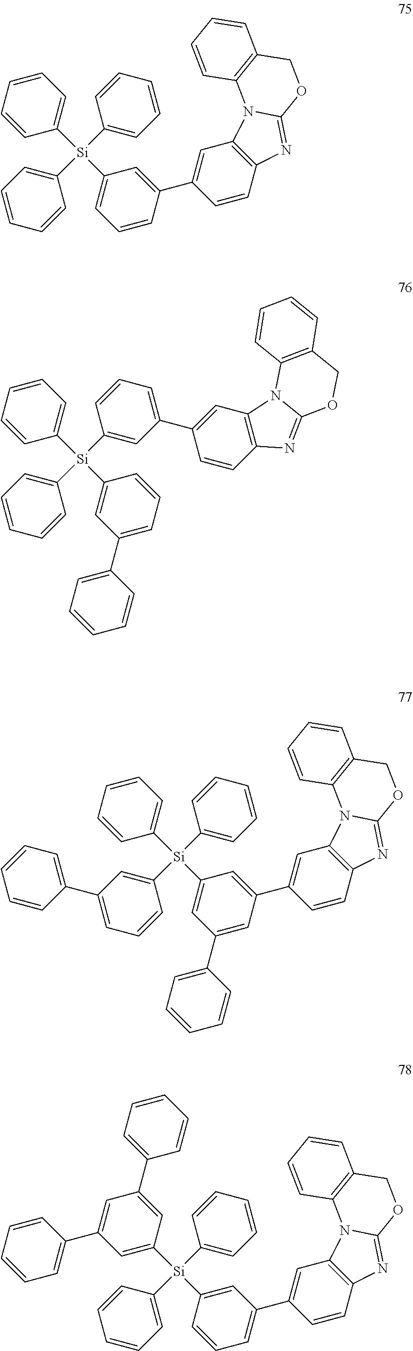 [SMILES: c1ccc([Si](c2ccccc2)(c2ccccc2)c2cccc(-c3ccc4nc5n(c4c3)-c3ccccc3CO5)c2)cc1 ; c1ccc(-c2cccc([Si](c3ccccc3)(c3ccccc3)c3cccc(-c4ccc5nc6n(c5c4)-c4ccccc4CO6)c3)c2)cc1 ; c1ccc(-c2cccc([Si](c3ccccc3)(c3ccccc3)c3cc(-c4ccccc4)cc(-c4ccc5nc6n(c5c4)-c4ccccc4CO6)c3)c2)cc1 ; c1ccc(-c2cc(-c3ccccc3)cc([Si](c3ccccc3)(c3ccccc3)c3cccc(-c4ccc5nc6n(c5c4)-c4ccccc4CO6)c3)c2)cc1]

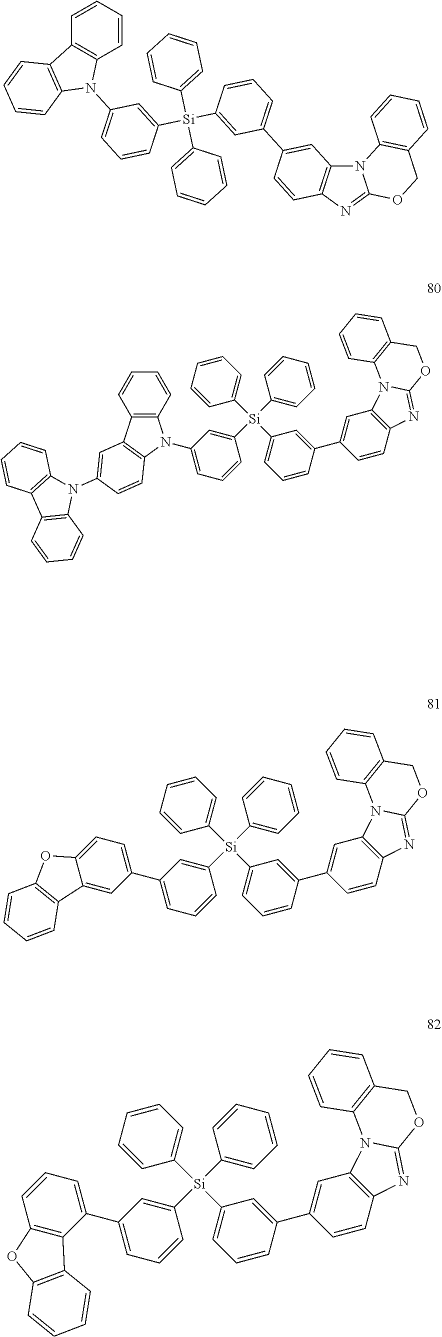 [SMILES: c1ccc([Si](c2ccccc2)(c2cccc(-c3ccc4nc5n(c4c3)-c3ccccc3CO5)c2)c2cccc(-n3c4ccccc4c4ccccc43)c2)cc1 ; c1ccc([Si](c2ccccc2)(c2cccc(-c3ccc4nc5n(c4c3)-c3ccccc3CO5)c2)c2cccc(-n3c4ccccc4c4cc(-n5c6ccccc6c6ccccc65)ccc43)c2)cc1 ; c1ccc([Si](c2ccccc2)(c2cccc(-c3ccc4oc5ccccc5c4c3)c2)c2cccc(-c3ccc4nc5n(c4c3)-c3ccccc3CO5)c2)cc1 ; c1ccc([Si](c2ccccc2)(c2cccc(-c3ccc4nc5n(c4c3)-c3ccccc3CO5)c2)c2cccc(-c3cccc4oc5ccccc5c34)c2)cc1]

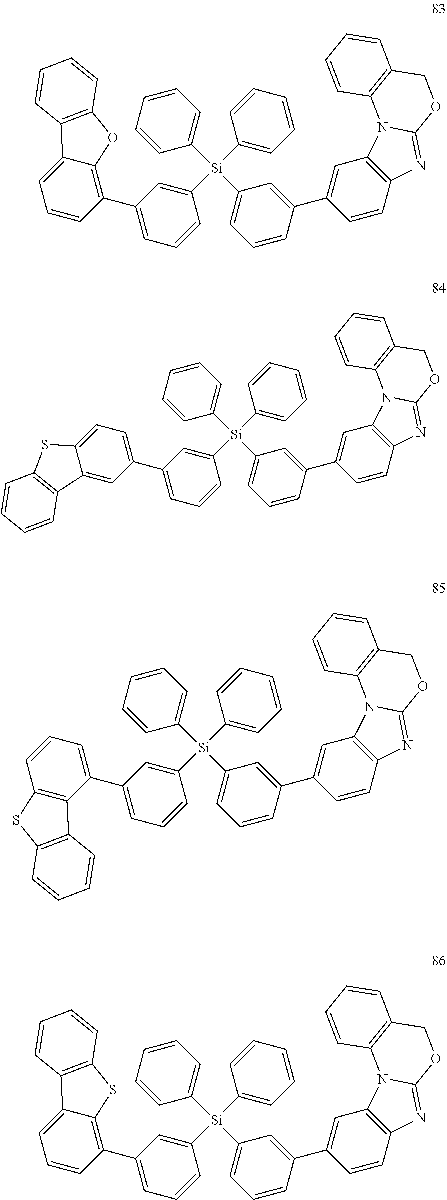 [SMILES: c1ccc([Si](c2ccccc2)(c2cccc(-c3ccc4nc5n(c4c3)-c3ccccc3CO5)c2)c2cccc(-c3cccc4c3oc3ccccc34)c2)cc1 ; c1ccc([Si](c2ccccc2)(c2cccc(-c3ccc4sc5ccccc5c4c3)c2)c2cccc(-c3ccc4nc5n(c4c3)-c3ccccc3CO5)c2)cc1 ; c1ccc([Si](c2ccccc2)(c2cccc(-c3ccc4nc5n(c4c3)-c3ccccc3CO5)c2)c2cccc(-c3cccc4sc5ccccc5c34)c2)cc1 ; c1ccc([Si](c2ccccc2)(c2cccc(-c3ccc4nc5n(c4c3)-c3ccccc3CO5)c2)c2cccc(-c3cccc4c3sc3ccccc34)c2)cc1]

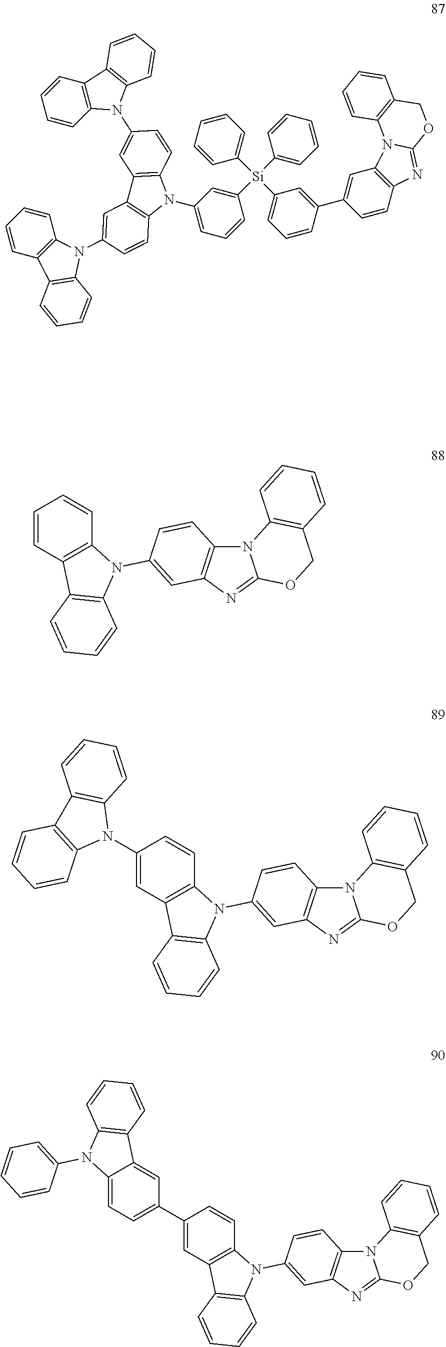 [SMILES: c1ccc([Si](c2ccccc2)(c2cccc(-c3ccc4nc5n(c4c3)-c3ccccc3CO5)c2)c2cccc(-n3c4ccc(-n5c6ccccc6c6ccccc65)cc4c4cc(-n5c6ccccc6c6ccccc65)ccc43)c2)cc1 ; c1ccc2c(c1)COc1nc3cc(-n4c5ccccc5c5ccccc54)ccc3n1-2 ; c1ccc2c(c1)COc1nc3cc(-n4c5ccccc5c5cc(-n6c7ccccc7c7ccccc76)ccc54)ccc3n1-2 ; c1ccc(-n2c3ccccc3c3cc(-c4ccc5c(c4)c4ccccc4n5-c4ccc5c(c4)nc4n5-c5ccccc5CO4)ccc32)cc1]

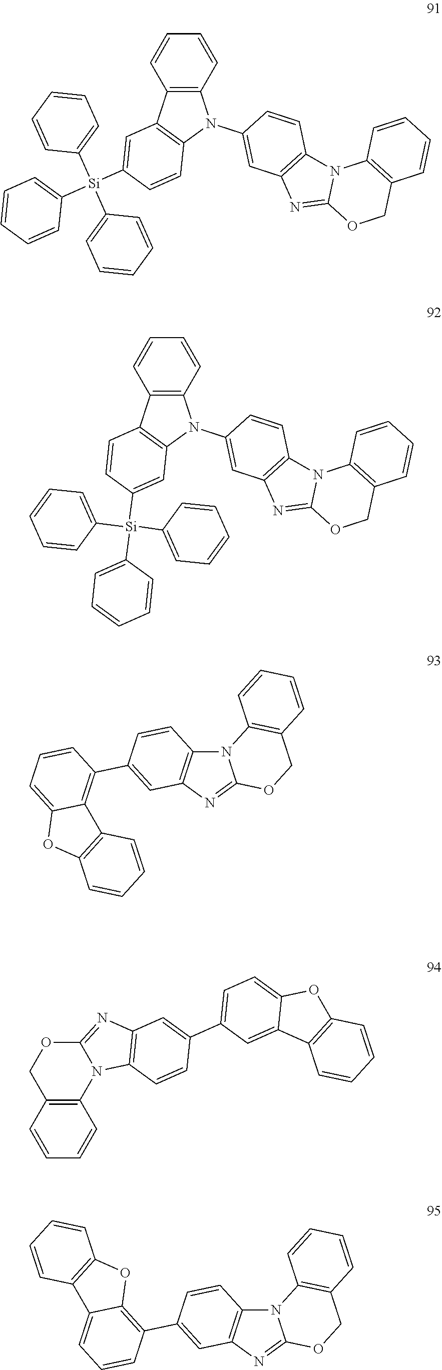 [SMILES: c1ccc([Si](c2ccccc2)(c2ccccc2)c2ccc3c(c2)c2ccccc2n3-c2ccc3c(c2)nc2n3-c3ccccc3CO2)cc1 ; c1ccc([Si](c2ccccc2)(c2ccccc2)c2ccc3c4ccccc4n(-c4ccc5c(c4)nc4n5-c5ccccc5CO4)c3c2)cc1 ; c1ccc2c(c1)COc1nc3cc(-c4cccc5oc6ccccc6c45)ccc3n1-2 ; c1ccc2c(c1)COc1nc3cc(-c4ccc5oc6ccccc6c5c4)ccc3n1-2 ; c1ccc2c(c1)COc1nc3cc(-c4cccc5c4oc4ccccc45)ccc3n1-2]

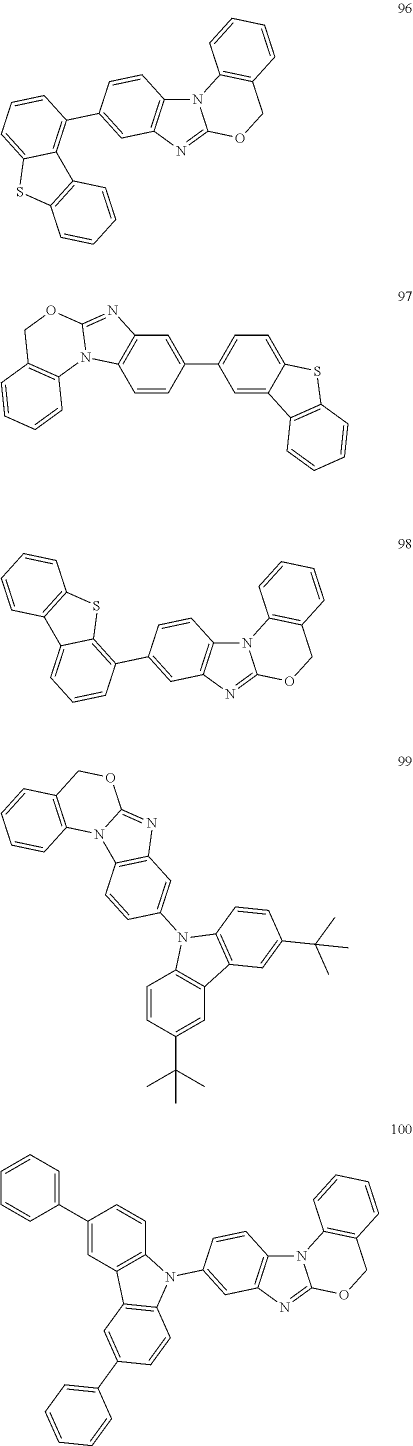 [SMILES: c1ccc2c(c1)COc1nc3cc(-c4cccc5sc6ccccc6c45)ccc3n1-2 ; c1ccc2c(c1)COc1nc3cc(-c4ccc5sc6ccccc6c5c4)ccc3n1-2 ; c1ccc2c(c1)COc1nc3cc(-c4cccc5c4sc4ccccc45)ccc3n1-2 ; CC(C)(C)c1ccc2c(c1)c1cc(C(C)(C)C)ccc1n2-c1ccc2c(c1)nc1n2-c2ccccc2CO1 ; c1ccc(-c2ccc3c(c2)c2cc(-c4ccccc4)ccc2n3-c2ccc3c(c2)nc2n3-c3ccccc3CO2)cc1]

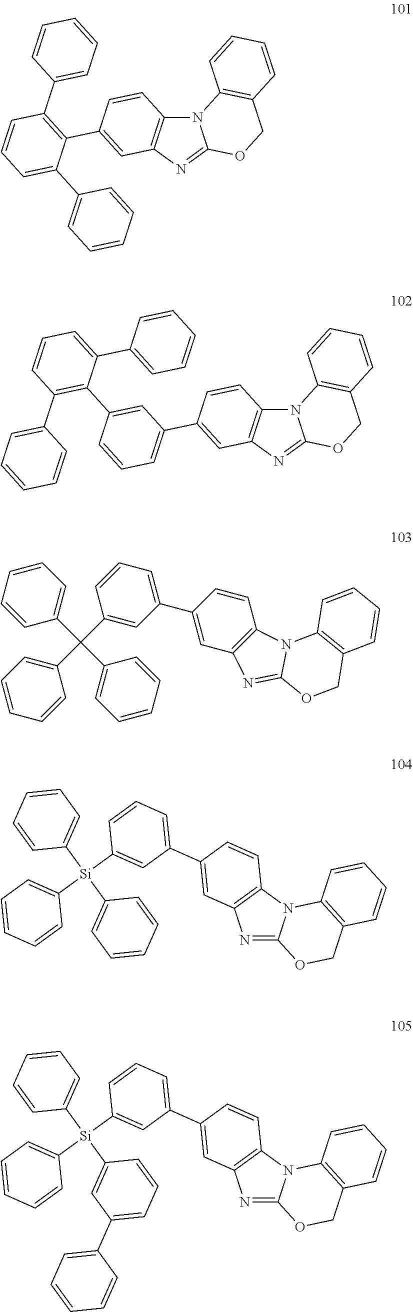 [SMILES: c1ccc(-c2cccc(-c3ccccc3)c2-c2ccc3c(c2)nc2n3-c3ccccc3CO2)cc1 ; c1ccc(-c2cccc(-c3ccccc3)c2-c2cccc(-c3ccc4c(c3)nc3n4-c4ccccc4CO3)c2)cc1 ; c1ccc(C(c2ccccc2)(c2ccccc2)c2cccc(-c3ccc4c(c3)nc3n4-c4ccccc4CO3)c2)cc1 ; c1ccc([Si](c2ccccc2)(c2ccccc2)c2cccc(-c3ccc4c(c3)nc3n4-c4ccccc4CO3)c2)cc1 ; c1ccc(-c2cccc([Si](c3ccccc3)(c3ccccc3)c3cccc(-c4ccc5c(c4)nc4n5-c5ccccc5CO4)c3)c2)cc1]

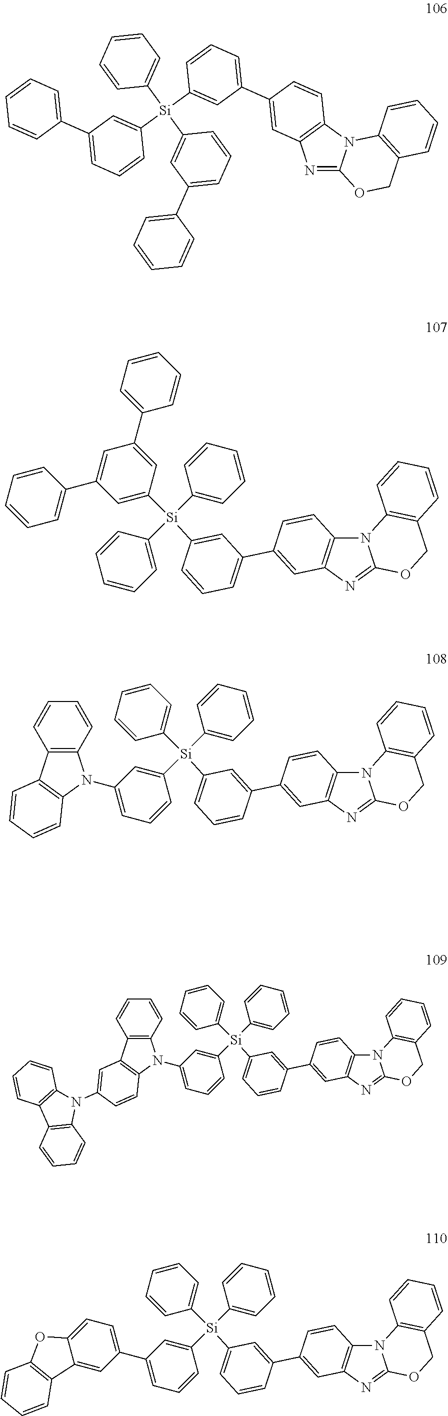

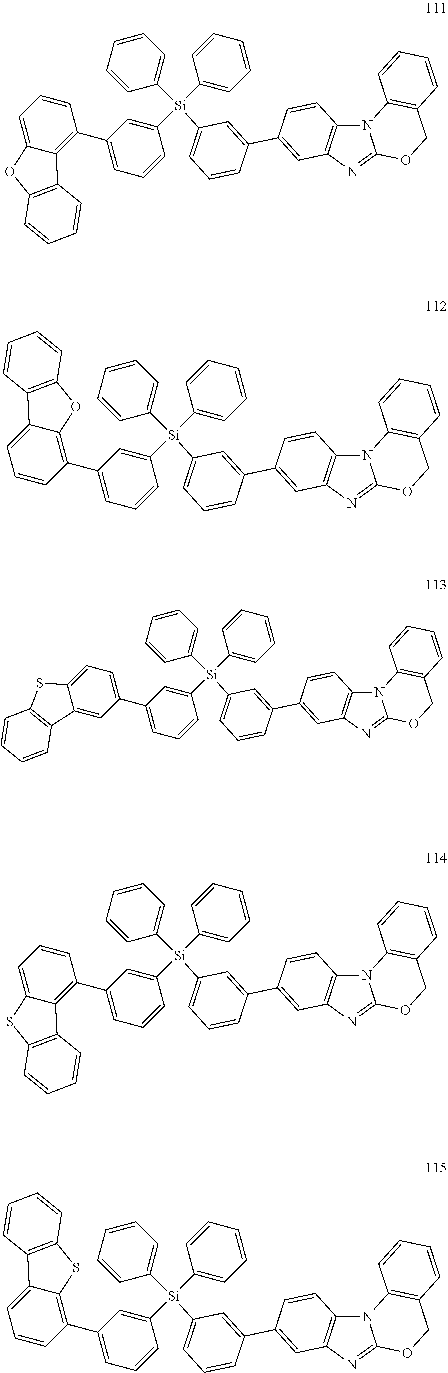 [SMILES: c1ccc([Si](c2ccccc2)(c2cccc(-c3ccc4c(c3)nc3n4-c4ccccc4CO3)c2)c2cccc(-c3cccc4oc5ccccc5c34)c2)cc1 ; c1ccc([Si](c2ccccc2)(c2cccc(-c3ccc4c(c3)nc3n4-c4ccccc4CO3)c2)c2cccc(-c3cccc4c3oc3ccccc34)c2)cc1 ; c1ccc([Si](c2ccccc2)(c2cccc(-c3ccc4c(c3)nc3n4-c4ccccc4CO3)c2)c2cccc(-c3ccc4sc5ccccc5c4c3)c2)cc1 ; c1ccc([Si](c2ccccc2)(c2cccc(-c3ccc4c(c3)nc3n4-c4ccccc4CO3)c2)c2cccc(-c3cccc4sc5ccccc5c34)c2)cc1 ; c1ccc([Si](c2ccccc2)(c2cccc(-c3ccc4c(c3)nc3n4-c4ccccc4CO3)c2)c2cccc(-c3cccc4c3sc3ccccc34)c2)cc1]

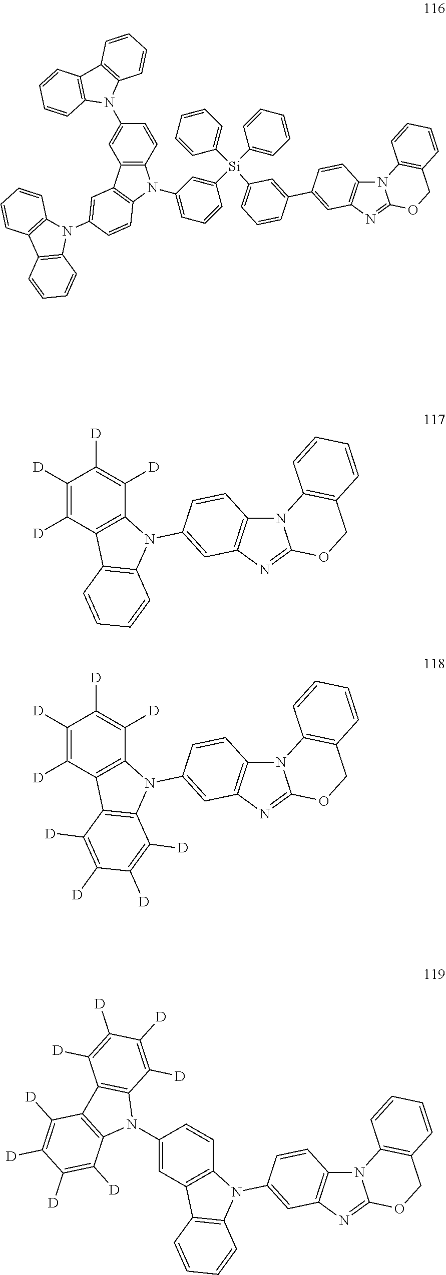 [SMILES: c1ccc([Si](c2ccccc2)(c2cccc(-c3ccc4c(c3)nc3n4-c4ccccc4CO3)c2)c2cccc(-n3c4ccc(-n5c6ccccc6c6ccccc65)cc4c4cc(-n5c6ccccc6c6ccccc65)ccc43)c2)cc1 ; [2H]c1c([2H])c([2H])c2c(c1[2H])c1ccccc1n2-c1ccc2c(c1)nc1n2-c2ccccc2CO1 ; [2H]c1c([2H])c([2H])c2c(c1[2H])c1c([2H])c([2H])c([2H])c([2H])c1n2-c1ccc2c(c1)nc1n2-c2ccccc2CO1 ; [2H]c1c([2H])c([2H])c2c(c1[2H])c1c([2H])c([2H])c([2H])c([2H])c1n2-c1ccc2c(c1)c1ccccc1n2-c1ccc2c(c1)nc1n2-c2ccccc2CO1]

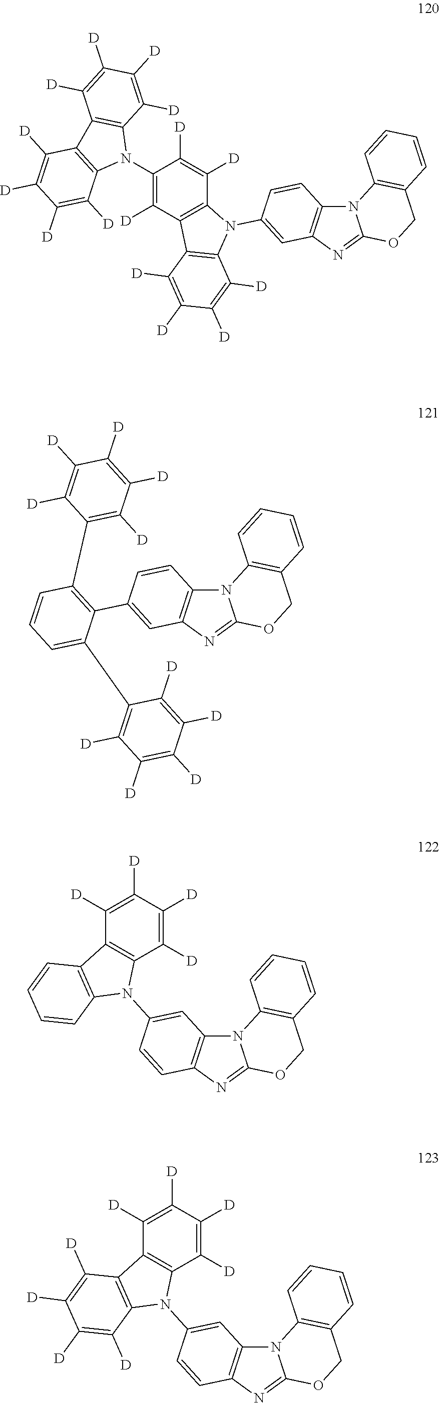 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc2c(c1)nc1n2-c2ccccc2CO1 ; [2H]c1c([2H])c([2H])c(-c2cccc(-c3c([2H])c([2H])c([2H])c([2H])c3[2H])c2-c2ccc3c(c2)nc2n3-c3ccccc3CO2)c([2H])c1[2H] ; [2H]c1c([2H])c([2H])c2c(c1[2H])c1ccccc1n2-c1ccc2nc3n(c2c1)-c1ccccc1CO3 ; [2H]c1c([2H])c([2H])c2c(c1[2H])c1c([2H])c([2H])c([2H])c([2H])c1n2-c1ccc2nc3n(c2c1)-c1ccccc1CO3]

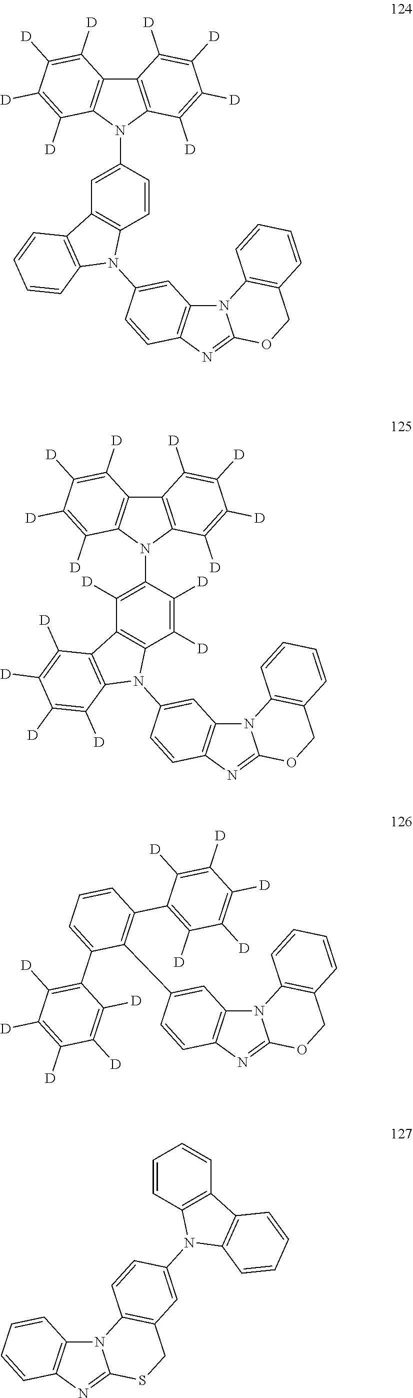 [SMILES: [2H]c1c([2H])c([2H])c2c(c1[2H])c1c([2H])c([2H])c([2H])c([2H])c1n2-c1ccc2c(c1)c1ccccc1n2-c1ccc2nc3n(c2c1)-c1ccccc1CO3 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc2nc3n(c2c1)-c1ccccc1CO3 ; [2H]c1c([2H])c([2H])c(-c2cccc(-c3c([2H])c([2H])c([2H])c([2H])c3[2H])c2-c2ccc3nc4n(c3c2)-c2ccccc2CO4)c([2H])c1[2H] ; c1ccc2c(c1)nc1n2-c2ccc(-n3c4ccccc4c4ccccc43)cc2CS1]

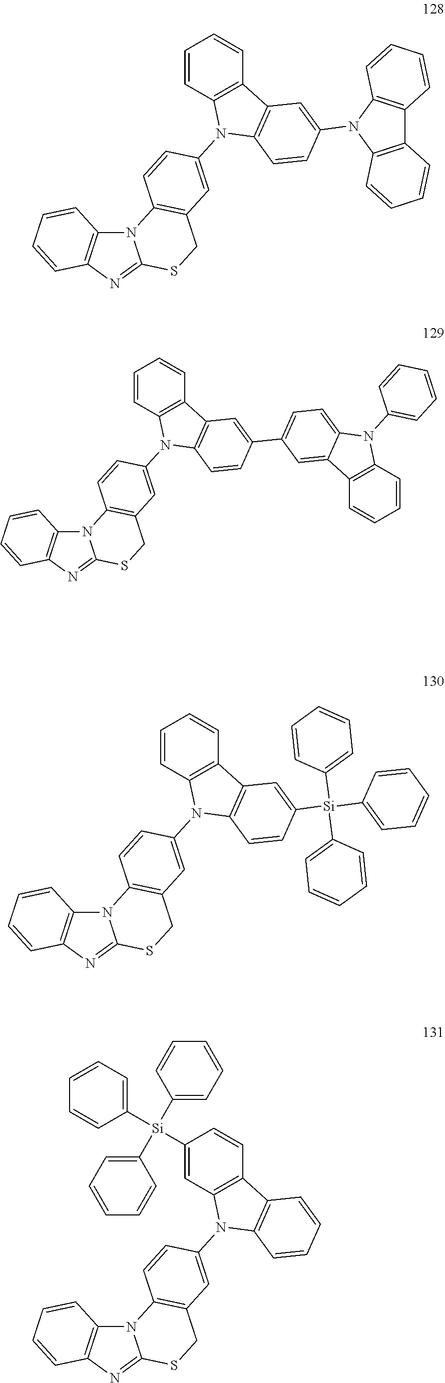 [SMILES: c1ccc2c(c1)nc1n2-c2ccc(-n3c4ccccc4c4cc(-n5c6ccccc6c6ccccc65)ccc43)cc2CS1 ; c1ccc(-n2c3ccccc3c3cc(-c4ccc5c(c4)c4ccccc4n5-c4ccc5c(c4)CSc4nc6ccccc6n4-5)ccc32)cc1 ; c1ccc([Si](c2ccccc2)(c2ccccc2)c2ccc3c(c2)c2ccccc2n3-c2ccc3c(c2)CSc2nc4ccccc4n2-3)cc1 ; c1ccc([Si](c2ccccc2)(c2ccccc2)c2ccc3c4ccccc4n(-c4ccc5c(c4)CSc4nc6ccccc6n4-5)c3c2)cc1]

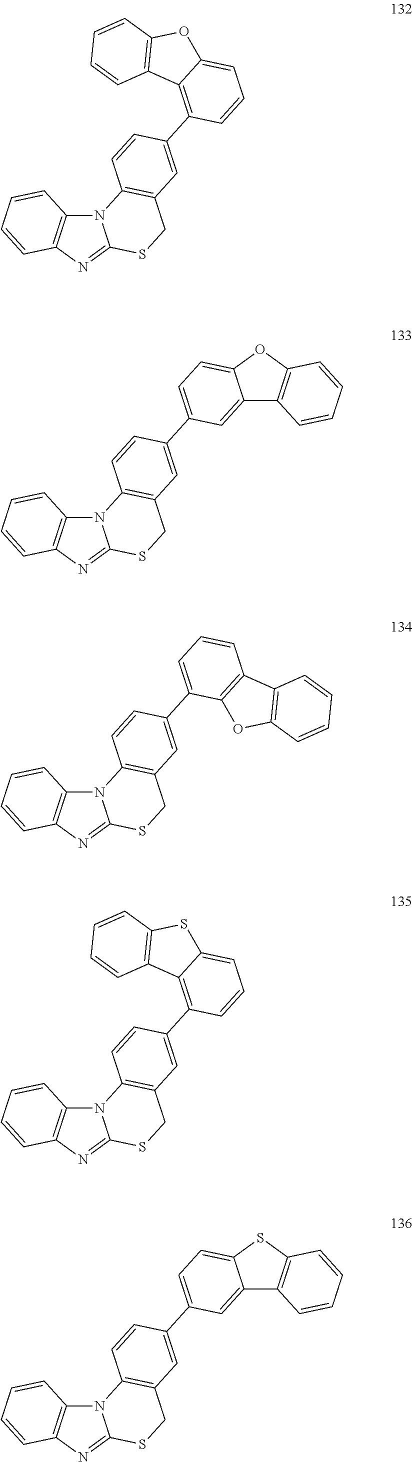 [SMILES: c1ccc2c(c1)nc1n2-c2ccc(-c3cccc4oc5ccccc5c34)cc2CS1 ; c1ccc2c(c1)nc1n2-c2ccc(-c3ccc4oc5ccccc5c4c3)cc2CS1 ; c1ccc2c(c1)nc1n2-c2ccc(-c3cccc4c3oc3ccccc34)cc2CS1 ; c1ccc2c(c1)nc1n2-c2ccc(-c3cccc4sc5ccccc5c34)cc2CS1 ; c1ccc2c(c1)nc1n2-c2ccc(-c3ccc4sc5ccccc5c4c3)cc2CS1]

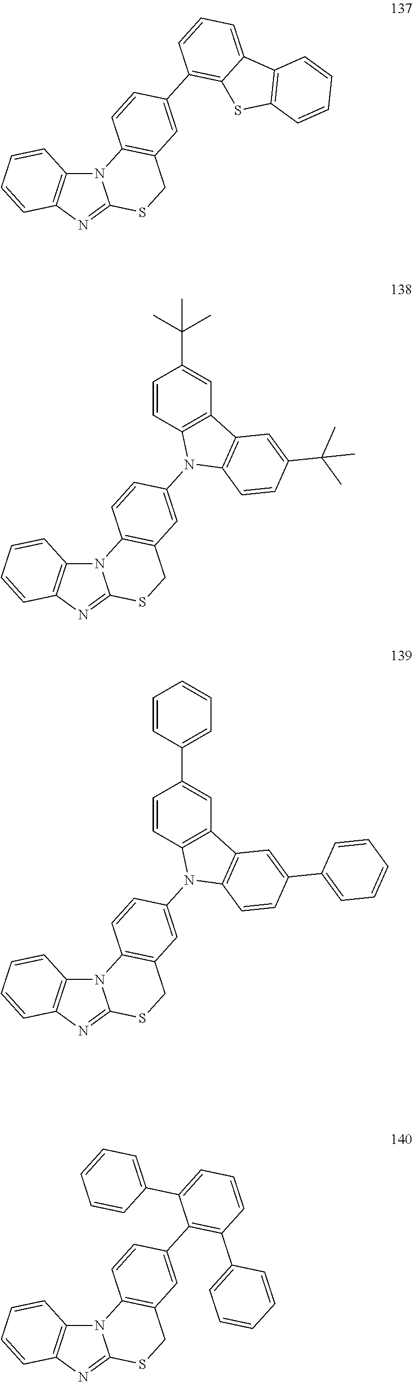 [SMILES: c1ccc2c(c1)nc1n2-c2ccc(-c3cccc4c3sc3ccccc34)cc2CS1 ; CC(C)(C)c1ccc2c(c1)c1cc(C(C)(C)C)ccc1n2-c1ccc2c(c1)CSc1nc3ccccc3n1-2 ; c1ccc(-c2ccc3c(c2)c2cc(-c4ccccc4)ccc2n3-c2ccc3c(c2)CSc2nc4ccccc4n2-3)cc1 ; c1ccc(-c2cccc(-c3ccccc3)c2-c2ccc3c(c2)CSc2nc4ccccc4n2-3)cc1]

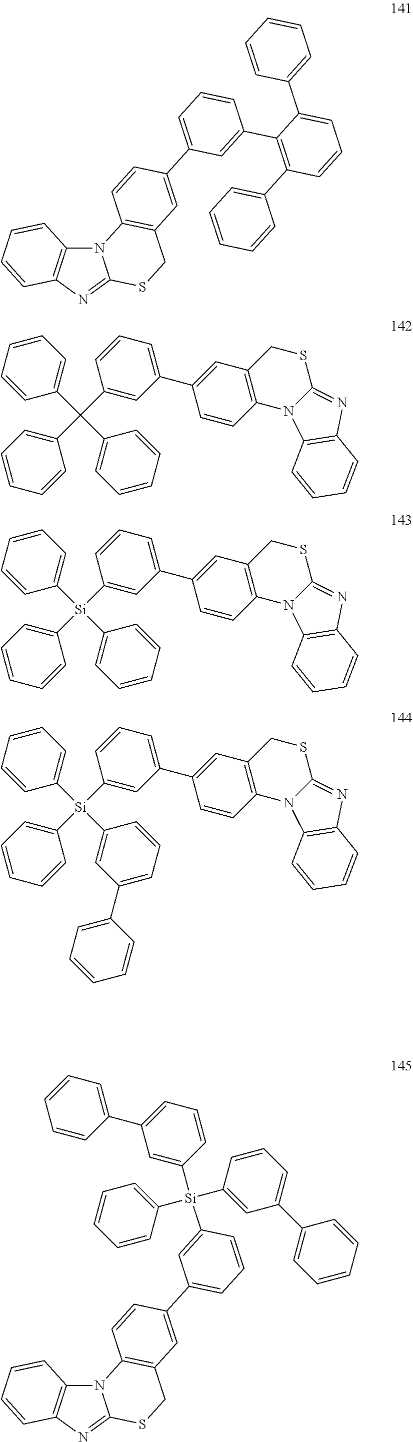

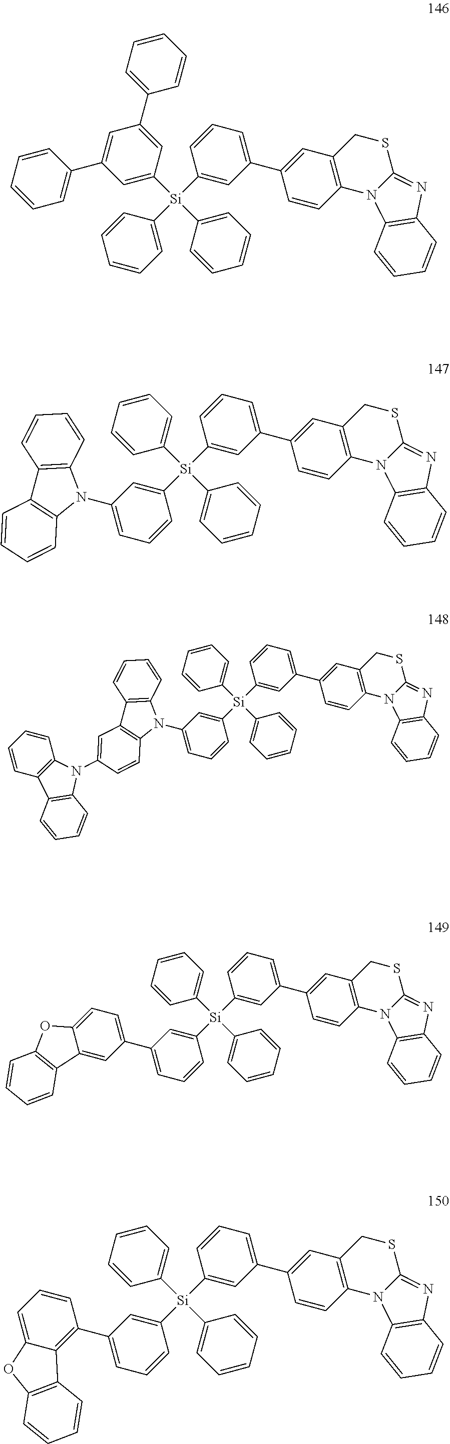

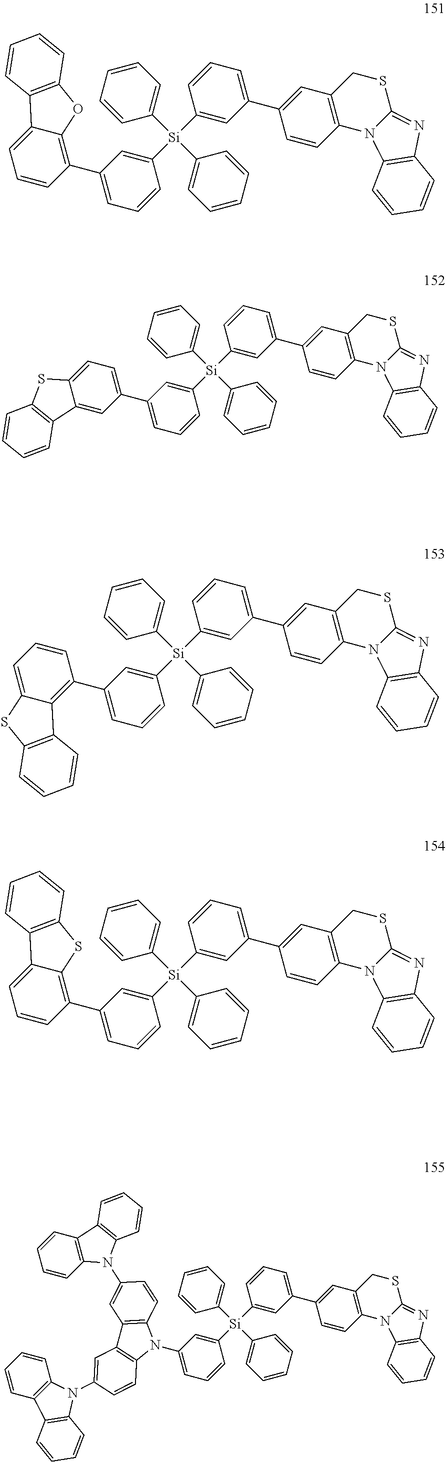

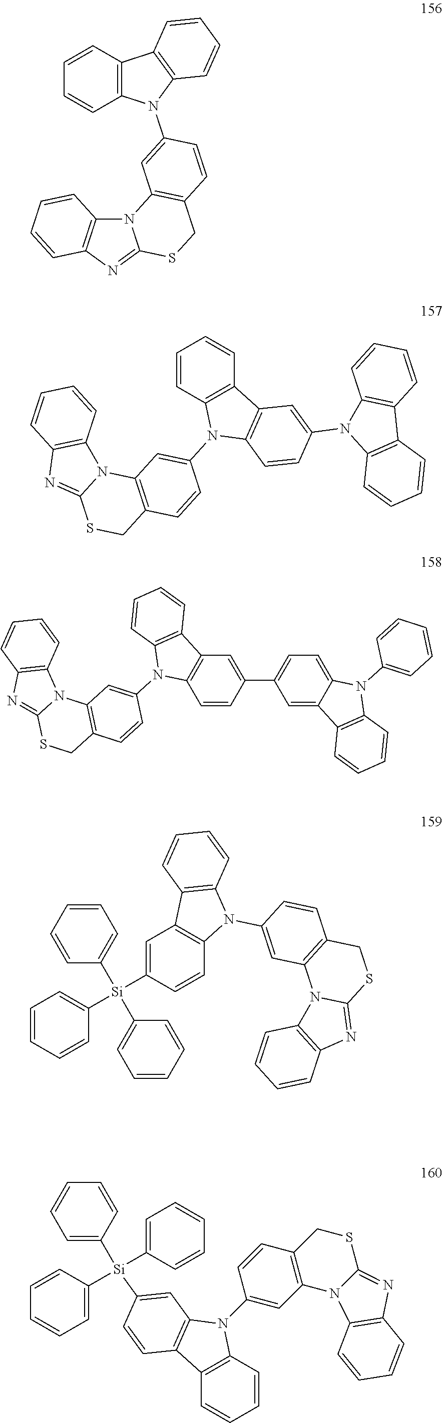 [SMILES: c1ccc2c(c1)nc1n2-c2cc(-n3c4ccccc4c4ccccc43)ccc2CS1 ; c1ccc2c(c1)nc1n2-c2cc(-n3c4ccccc4c4cc(-n5c6ccccc6c6ccccc65)ccc43)ccc2CS1 ; c1ccc(-n2c3ccccc3c3cc(-c4ccc5c(c4)c4ccccc4n5-c4ccc5c(c4)-n4c(nc6ccccc64)SC5)ccc32)cc1 ; c1ccc([Si](c2ccccc2)(c2ccccc2)c2ccc3c(c2)c2ccccc2n3-c2ccc3c(c2)-n2c(nc4ccccc42)SC3)cc1 ; c1ccc([Si](c2ccccc2)(c2ccccc2)c2ccc3c4ccccc4n(-c4ccc5c(c4)-n4c(nc6ccccc64)SC5)c3c2)cc1]

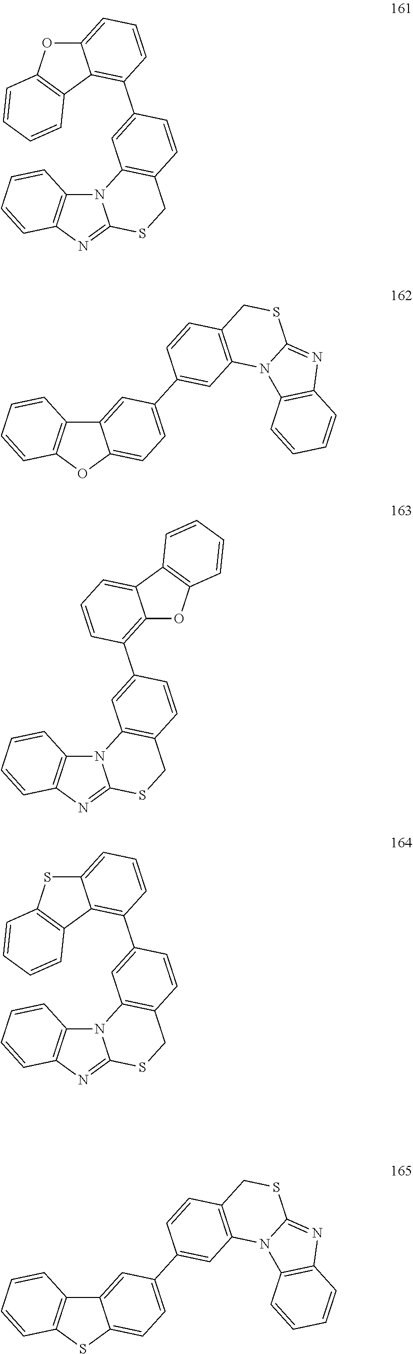 [SMILES: c1ccc2c(c1)nc1n2-c2cc(-c3cccc4oc5ccccc5c34)ccc2CS1 ; c1ccc2c(c1)nc1n2-c2cc(-c3ccc4oc5ccccc5c4c3)ccc2CS1 ; c1ccc2c(c1)nc1n2-c2cc(-c3cccc4c3oc3ccccc34)ccc2CS1 ; c1ccc2c(c1)nc1n2-c2cc(-c3cccc4sc5ccccc5c34)ccc2CS1 ; c1ccc2c(c1)nc1n2-c2cc(-c3ccc4sc5ccccc5c4c3)ccc2CS1]

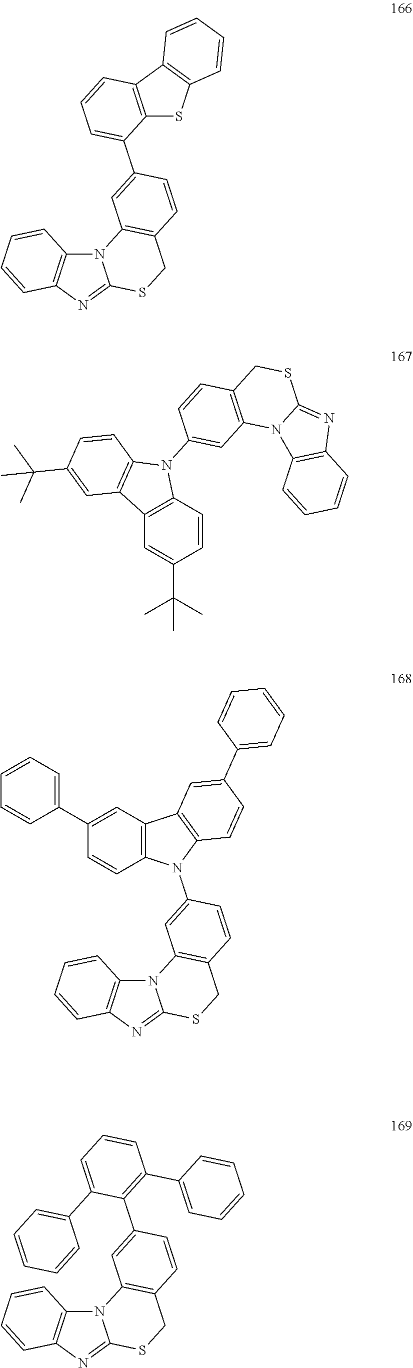 [SMILES: c1ccc2c(c1)nc1n2-c2cc(-c3cccc4c3sc3ccccc34)ccc2CS1 ; CC(C)(C)c1ccc2c(c1)c1cc(C(C)(C)C)ccc1n2-c1ccc2c(c1)-n1c(nc3ccccc31)SC2 ; c1ccc(-c2ccc3c(c2)c2cc(-c4ccccc4)ccc2n3-c2ccc3c(c2)-n2c(nc4ccccc42)SC3)cc1 ; c1ccc(-c2cccc(-c3ccccc3)c2-c2ccc3c(c2)-n2c(nc4ccccc42)SC3)cc1]

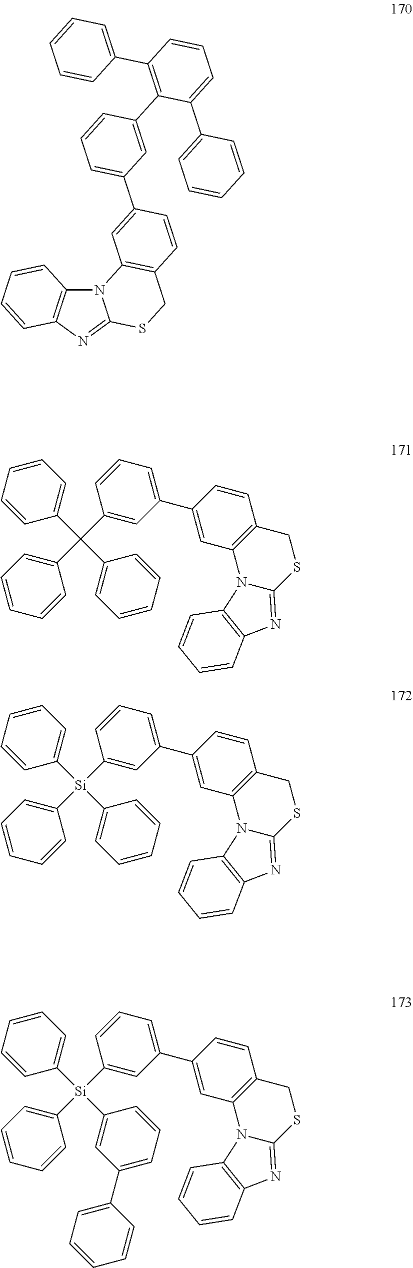 [SMILES: c1ccc(-c2cccc(-c3ccccc3)c2-c2cccc(-c3ccc4c(c3)-n3c(nc5ccccc53)SC4)c2)cc1 ; c1ccc(C(c2ccccc2)(c2ccccc2)c2cccc(-c3ccc4c(c3)-n3c(nc5ccccc53)SC4)c2)cc1 ; c1ccc([Si](c2ccccc2)(c2ccccc2)c2cccc(-c3ccc4c(c3)-n3c(nc5ccccc53)SC4)c2)cc1 ; c1ccc(-c2cccc([Si](c3ccccc3)(c3ccccc3)c3cccc(-c4ccc5c(c4)-n4c(nc6ccccc64)SC5)c3)c2)cc1]

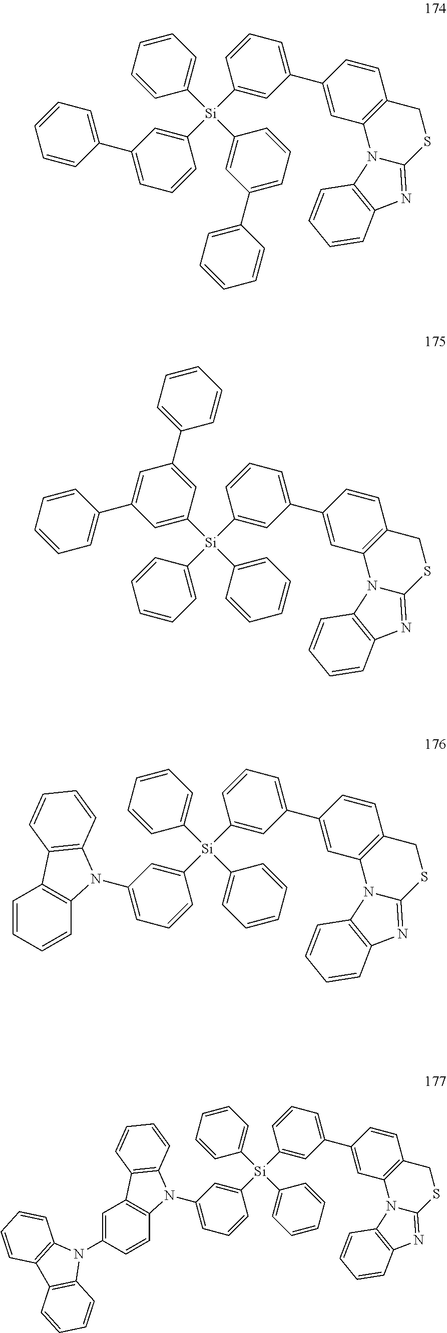 [SMILES: c1ccc(-c2cccc([Si](c3ccccc3)(c3cccc(-c4ccccc4)c3)c3cccc(-c4ccc5c(c4)-n4c(nc6ccccc64)SC5)c3)c2)cc1 ; c1ccc(-c2cc(-c3ccccc3)cc([Si](c3ccccc3)(c3ccccc3)c3cccc(-c4ccc5c(c4)-n4c(nc6ccccc64)SC5)c3)c2)cc1 ; c1ccc([Si](c2ccccc2)(c2cccc(-c3ccc4c(c3)-n3c(nc5ccccc53)SC4)c2)c2cccc(-n3c4ccccc4c4ccccc43)c2)cc1 ; c1ccc([Si](c2ccccc2)(c2cccc(-c3ccc4c(c3)-n3c(nc5ccccc53)SC4)c2)c2cccc(-n3c4ccccc4c4cc(-n5c6ccccc6c6ccccc65)ccc43)c2)cc1]

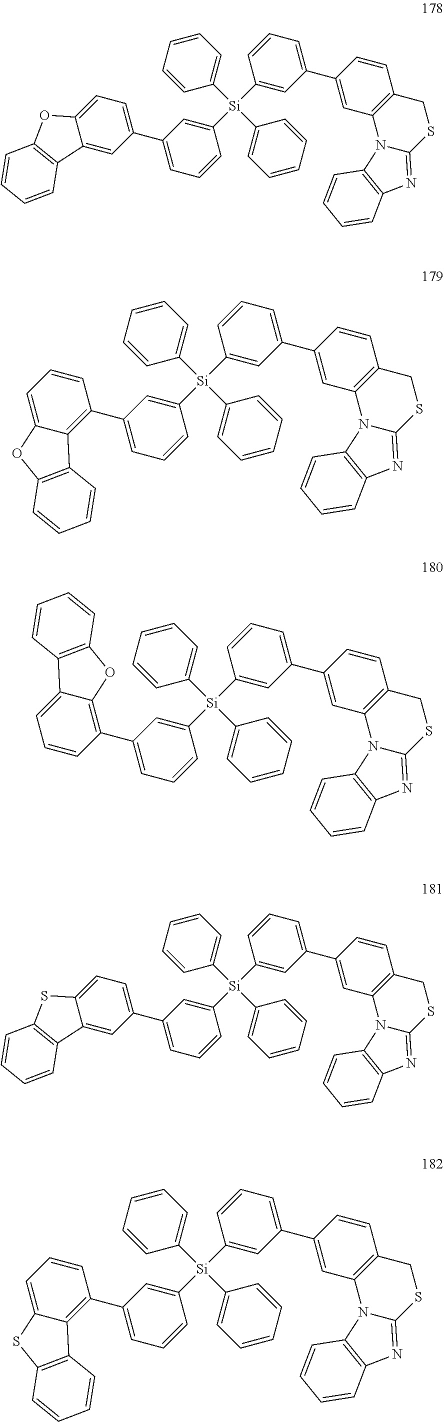 [SMILES: c1ccc([Si](c2ccccc2)(c2cccc(-c3ccc4c(c3)-n3c(nc5ccccc53)SC4)c2)c2cccc(-c3ccc4oc5ccccc5c4c3)c2)cc1 ; c1ccc([Si](c2ccccc2)(c2cccc(-c3ccc4c(c3)-n3c(nc5ccccc53)SC4)c2)c2cccc(-c3cccc4oc5ccccc5c34)c2)cc1 ; c1ccc([Si](c2ccccc2)(c2cccc(-c3ccc4c(c3)-n3c(nc5ccccc53)SC4)c2)c2cccc(-c3cccc4c3oc3ccccc34)c2)cc1 ; c1ccc([Si](c2ccccc2)(c2cccc(-c3ccc4c(c3)-n3c(nc5ccccc53)SC4)c2)c2cccc(-c3ccc4sc5ccccc5c4c3)c2)cc1 ; c1ccc([Si](c2ccccc2)(c2cccc(-c3ccc4c(c3)-n3c(nc5ccccc53)SC4)c2)c2cccc(-c3cccc4sc5ccccc5c34)c2)cc1]

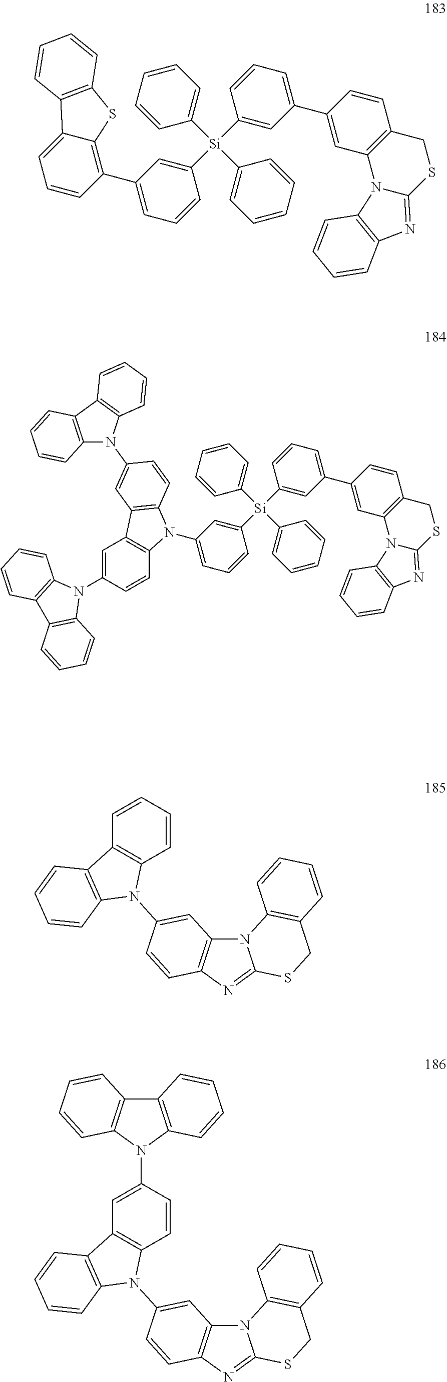 [SMILES: c1ccc([Si](c2ccccc2)(c2cccc(-c3ccc4c(c3)-n3c(nc5ccccc53)SC4)c2)c2cccc(-c3cccc4c3sc3ccccc34)c2)cc1 ; c1ccc([Si](c2ccccc2)(c2cccc(-c3ccc4c(c3)-n3c(nc5ccccc53)SC4)c2)c2cccc(-n3c4ccc(-n5c6ccccc6c6ccccc65)cc4c4cc(-n5c6ccccc6c6ccccc65)ccc43)c2)cc1 ; c1ccc2c(c1)CSc1nc3ccc(-n4c5ccccc5c5ccccc54)cc3n1-2 ; c1ccc2c(c1)CSc1nc3ccc(-n4c5ccccc5c5cc(-n6c7ccccc7c7ccccc76)ccc54)cc3n1-2]

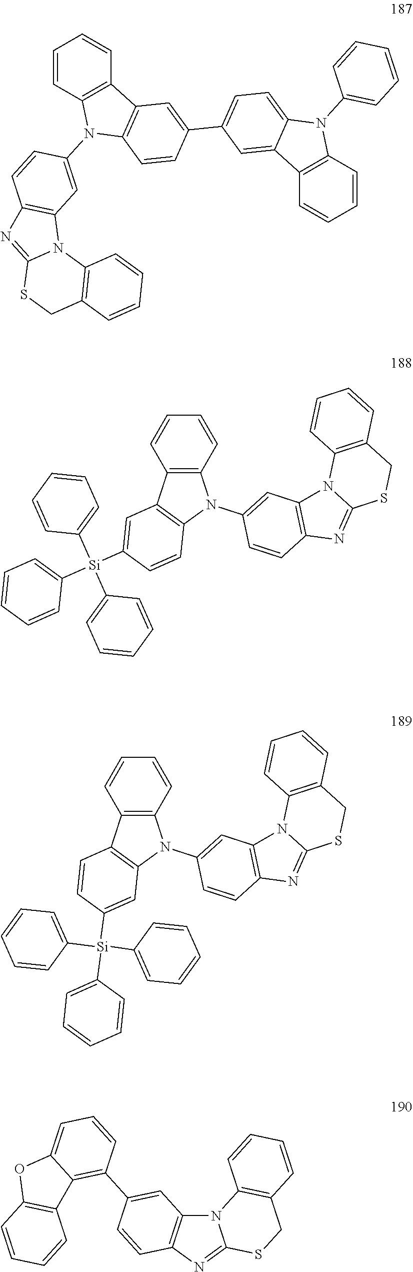 [SMILES: c1ccc(-n2c3ccccc3c3cc(-c4ccc5c(c4)c4ccccc4n5-c4ccc5nc6n(c5c4)-c4ccccc4CS6)ccc32)cc1 ; c1ccc([Si](c2ccccc2)(c2ccccc2)c2ccc3c(c2)c2ccccc2n3-c2ccc3nc4n(c3c2)-c2ccccc2CS4)cc1 ; c1ccc([Si](c2ccccc2)(c2ccccc2)c2ccc3c4ccccc4n(-c4ccc5nc6n(c5c4)-c4ccccc4CS6)c3c2)cc1 ; c1ccc2c(c1)CSc1nc3ccc(-c4cccc5oc6ccccc6c45)cc3n1-2]

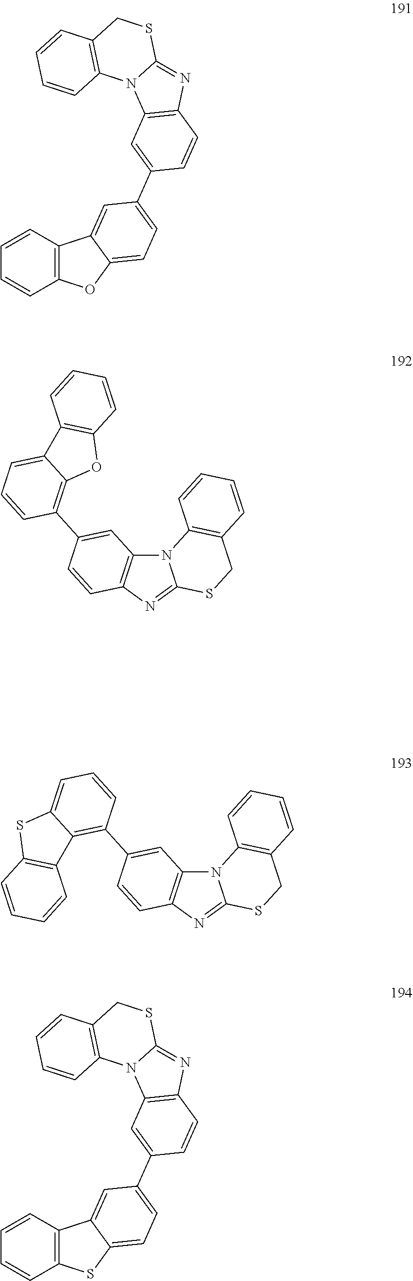 [SMILES: c1ccc2c(c1)CSc1nc3ccc(-c4ccc5oc6ccccc6c5c4)cc3n1-2 ; c1ccc2c(c1)CSc1nc3ccc(-c4cccc5c4oc4ccccc45)cc3n1-2 ; c1ccc2c(c1)CSc1nc3ccc(-c4cccc5sc6ccccc6c45)cc3n1-2 ; c1ccc2c(c1)CSc1nc3ccc(-c4ccc5sc6ccccc6c5c4)cc3n1-2]

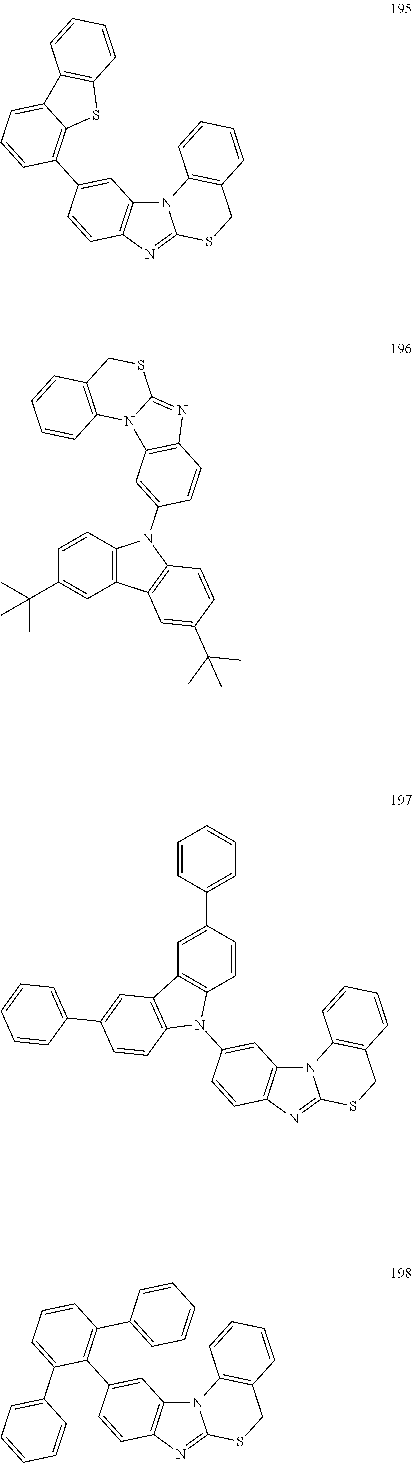 [SMILES: c1ccc2c(c1)CSc1nc3ccc(-c4cccc5c4sc4ccccc45)cc3n1-2 ; CC(C)(C)c1ccc2c(c1)c1cc(C(C)(C)C)ccc1n2-c1ccc2nc3n(c2c1)-c1ccccc1CS3 ; c1ccc(-c2ccc3c(c2)c2cc(-c4ccccc4)ccc2n3-c2ccc3nc4n(c3c2)-c2ccccc2CS4)cc1 ; c1ccc(-c2cccc(-c3ccccc3)c2-c2ccc3nc4n(c3c2)-c2ccccc2CS4)cc1]

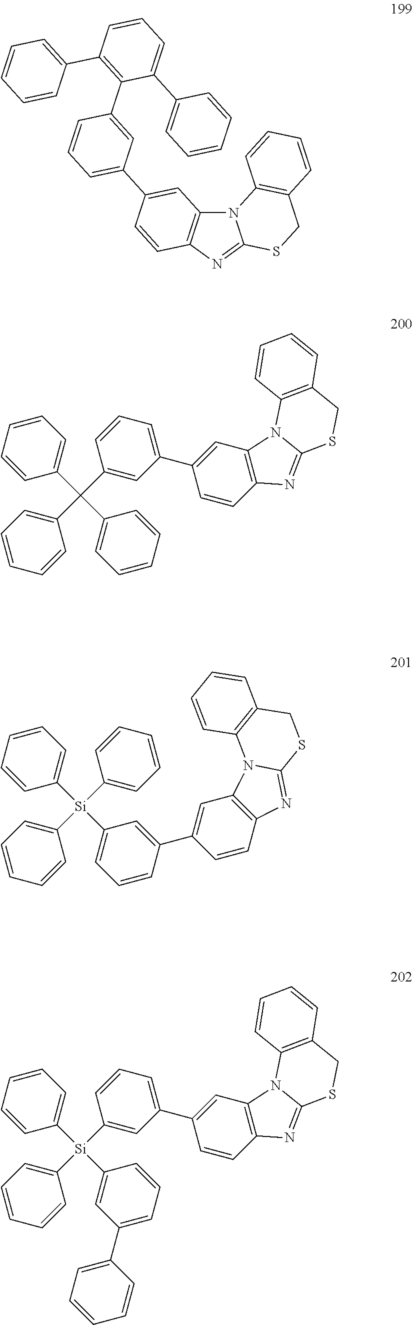 [SMILES: c1ccc(-c2cccc(-c3ccccc3)c2-c2cccc(-c3ccc4nc5n(c4c3)-c3ccccc3CS5)c2)cc1 ; c1ccc(C(c2ccccc2)(c2ccccc2)c2cccc(-c3ccc4nc5n(c4c3)-c3ccccc3CS5)c2)cc1 ; c1ccc([Si](c2ccccc2)(c2ccccc2)c2cccc(-c3ccc4nc5n(c4c3)-c3ccccc3CS5)c2)cc1 ; c1ccc(-c2cccc([Si](c3ccccc3)(c3ccccc3)c3cccc(-c4ccc5nc6n(c5c4)-c4ccccc4CS6)c3)c2)cc1]

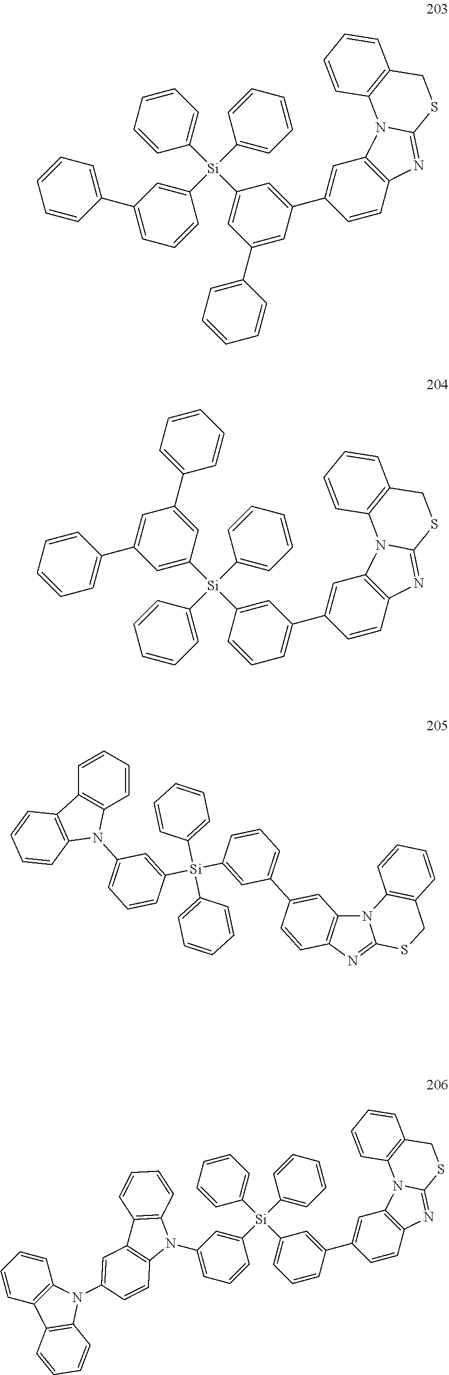 [SMILES: c1ccc(-c2cccc([Si](c3ccccc3)(c3ccccc3)c3cc(-c4ccccc4)cc(-c4ccc5nc6n(c5c4)-c4ccccc4CS6)c3)c2)cc1 ; c1ccc(-c2cc(-c3ccccc3)cc([Si](c3ccccc3)(c3ccccc3)c3cccc(-c4ccc5nc6n(c5c4)-c4ccccc4CS6)c3)c2)cc1 ; c1ccc([Si](c2ccccc2)(c2cccc(-c3ccc4nc5n(c4c3)-c3ccccc3CS5)c2)c2cccc(-n3c4ccccc4c4ccccc43)c2)cc1 ; c1ccc([Si](c2ccccc2)(c2cccc(-c3ccc4nc5n(c4c3)-c3ccccc3CS5)c2)c2cccc(-n3c4ccccc4c4cc(-n5c6ccccc6c6ccccc65)ccc43)c2)cc1]

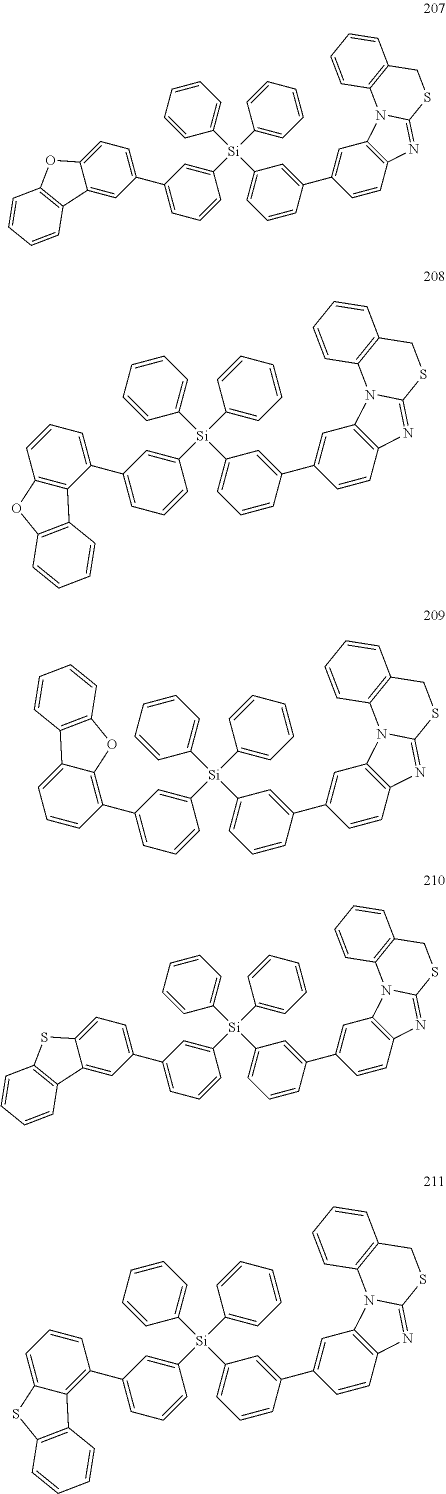 [SMILES: c1ccc([Si](c2ccccc2)(c2cccc(-c3ccc4oc5ccccc5c4c3)c2)c2cccc(-c3ccc4nc5n(c4c3)-c3ccccc3CS5)c2)cc1 ; c1ccc([Si](c2ccccc2)(c2cccc(-c3ccc4nc5n(c4c3)-c3ccccc3CS5)c2)c2cccc(-c3cccc4oc5ccccc5c34)c2)cc1 ; c1ccc([Si](c2ccccc2)(c2cccc(-c3ccc4nc5n(c4c3)-c3ccccc3CS5)c2)c2cccc(-c3cccc4c3oc3ccccc34)c2)cc1 ; c1ccc([Si](c2ccccc2)(c2cccc(-c3ccc4sc5ccccc5c4c3)c2)c2cccc(-c3ccc4nc5n(c4c3)-c3ccccc3CS5)c2)cc1 ; c1ccc([Si](c2ccccc2)(c2cccc(-c3ccc4nc5n(c4c3)-c3ccccc3CS5)c2)c2cccc(-c3cccc4sc5ccccc5c34)c2)cc1]

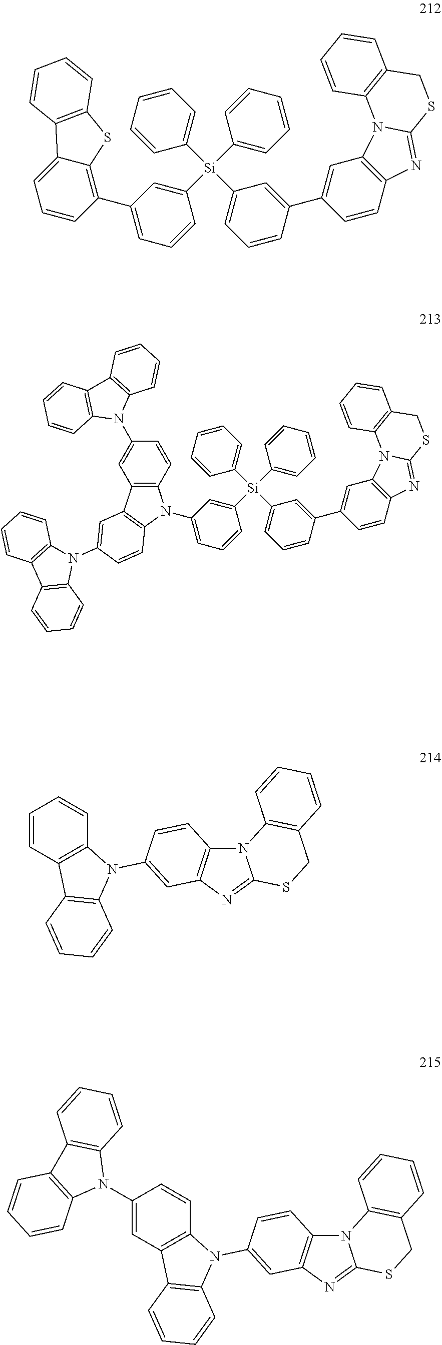 [SMILES: c1ccc([Si](c2ccccc2)(c2cccc(-c3ccc4nc5n(c4c3)-c3ccccc3CS5)c2)c2cccc(-c3cccc4c3sc3ccccc34)c2)cc1 ; c1ccc([Si](c2ccccc2)(c2cccc(-c3ccc4nc5n(c4c3)-c3ccccc3CS5)c2)c2cccc(-n3c4ccc(-n5c6ccccc6c6ccccc65)cc4c4cc(-n5c6ccccc6c6ccccc65)ccc43)c2)cc1 ; c1ccc2c(c1)CSc1nc3cc(-n4c5ccccc5c5ccccc54)ccc3n1-2 ; c1ccc2c(c1)CSc1nc3cc(-n4c5ccccc5c5cc(-n6c7ccccc7c7ccccc76)ccc54)ccc3n1-2]

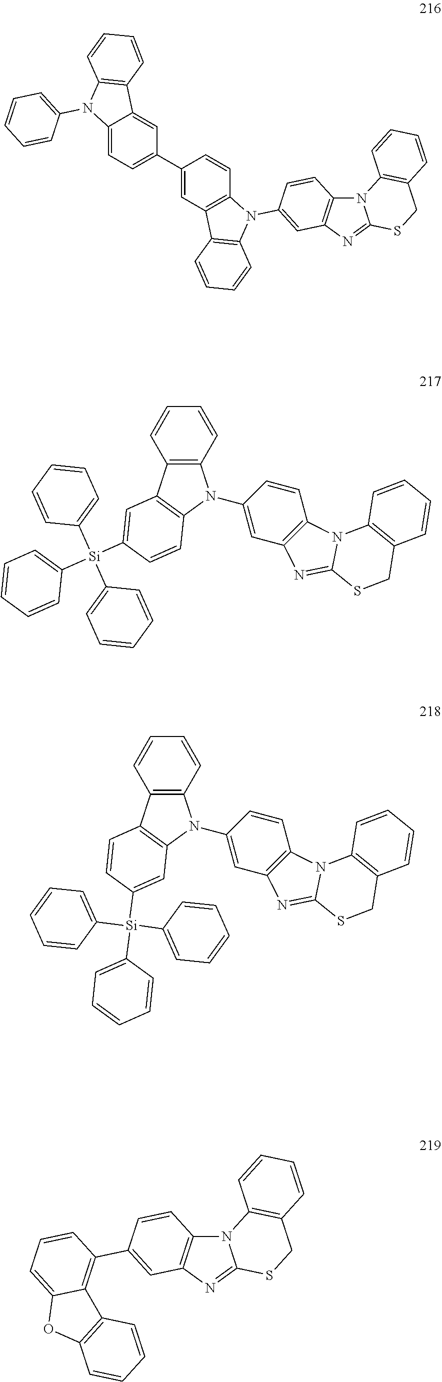

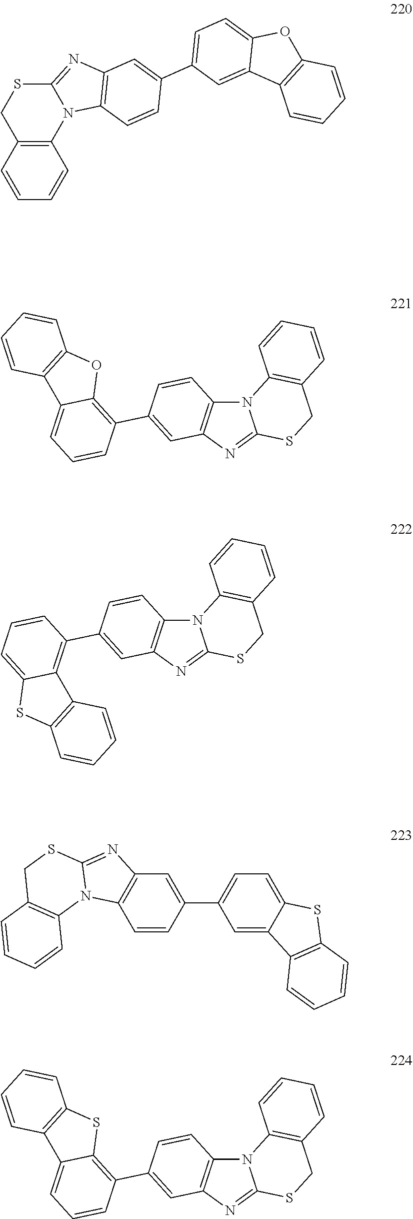 [SMILES: c1ccc2c(c1)CSc1nc3cc(-c4ccc5oc6ccccc6c5c4)ccc3n1-2 ; c1ccc2c(c1)CSc1nc3cc(-c4cccc5c4oc4ccccc45)ccc3n1-2 ; c1ccc2c(c1)CSc1nc3cc(-c4cccc5sc6ccccc6c45)ccc3n1-2 ; c1ccc2c(c1)CSc1nc3cc(-c4ccc5sc6ccccc6c5c4)ccc3n1-2 ; c1ccc2c(c1)CSc1nc3cc(-c4cccc5c4sc4ccccc45)ccc3n1-2]

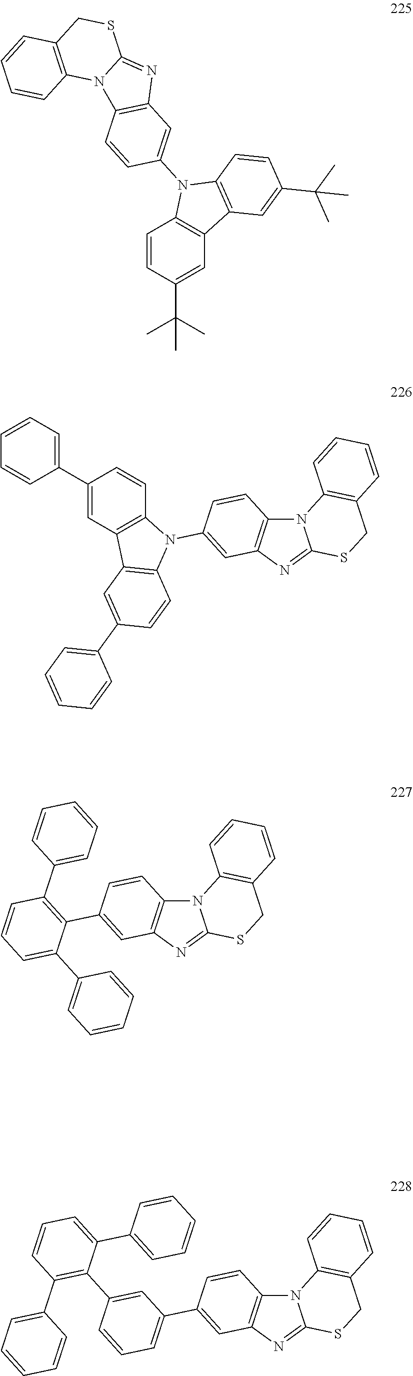 [SMILES: CC(C)(C)c1ccc2c(c1)c1cc(C(C)(C)C)ccc1n2-c1ccc2c(c1)nc1n2-c2ccccc2CS1 ; c1ccc(-c2ccc3c(c2)c2cc(-c4ccccc4)ccc2n3-c2ccc3c(c2)nc2n3-c3ccccc3CS2)cc1 ; c1ccc(-c2cccc(-c3ccccc3)c2-c2ccc3c(c2)nc2n3-c3ccccc3CS2)cc1 ; c1ccc(-c2cccc(-c3ccccc3)c2-c2cccc(-c3ccc4c(c3)nc3n4-c4ccccc4CS3)c2)cc1]

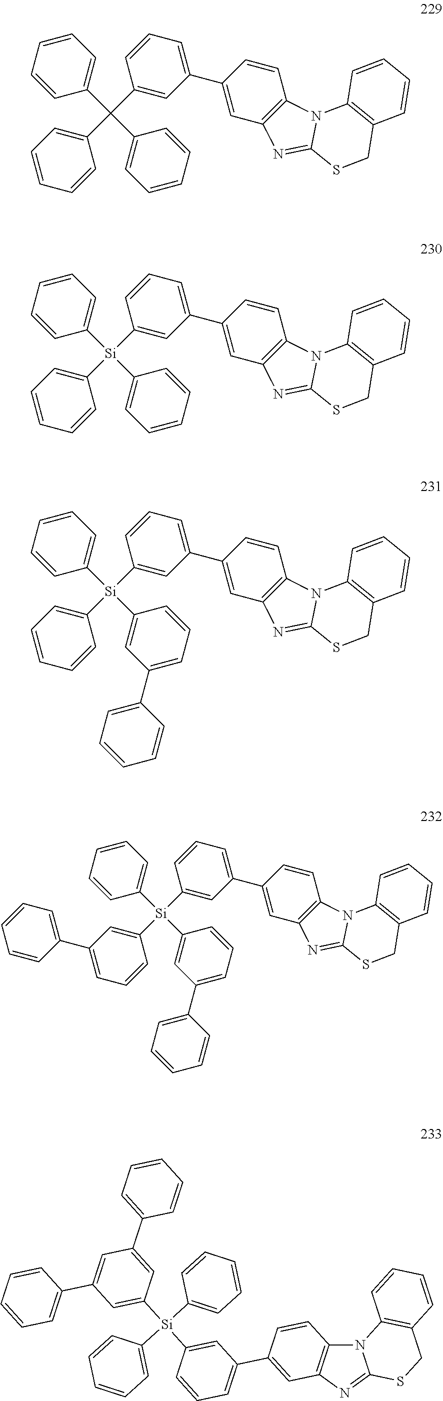 [SMILES: c1ccc(C(c2ccccc2)(c2ccccc2)c2cccc(-c3ccc4c(c3)nc3n4-c4ccccc4CS3)c2)cc1 ; c1ccc([Si](c2ccccc2)(c2ccccc2)c2cccc(-c3ccc4c(c3)nc3n4-c4ccccc4CS3)c2)cc1 ; c1ccc(-c2cccc([Si](c3ccccc3)(c3ccccc3)c3cccc(-c4ccc5c(c4)nc4n5-c5ccccc5CS4)c3)c2)cc1 ; c1ccc(-c2cccc([Si](c3ccccc3)(c3cccc(-c4ccccc4)c3)c3cccc(-c4ccc5c(c4)nc4n5-c5ccccc5CS4)c3)c2)cc1 ; c1ccc(-c2cc(-c3ccccc3)cc([Si](c3ccccc3)(c3ccccc3)c3cccc(-c4ccc5c(c4)nc4n5-c5ccccc5CS4)c3)c2)cc1]

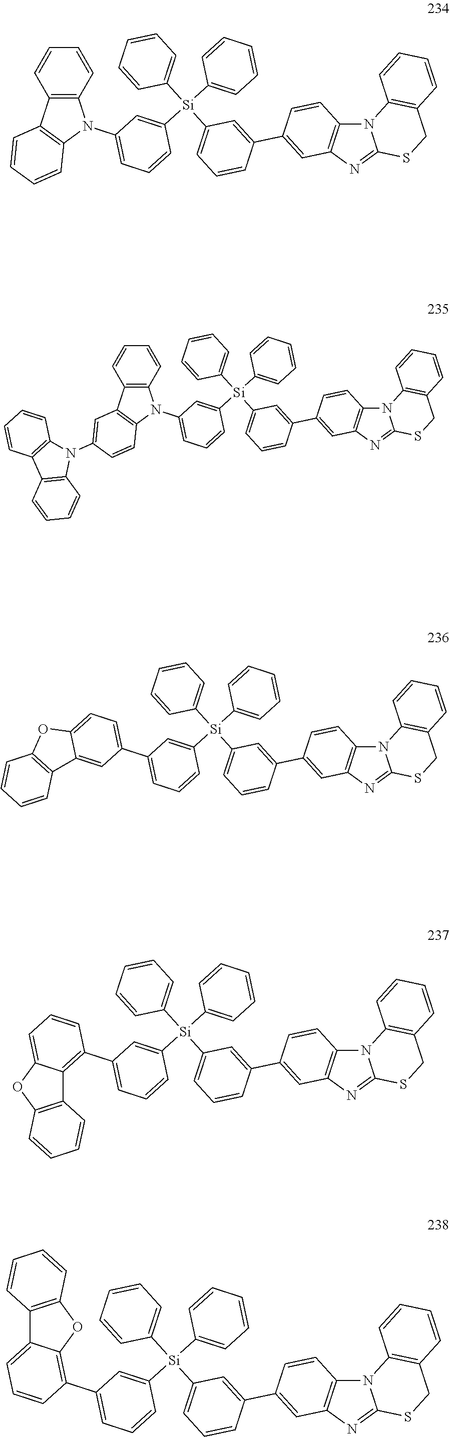

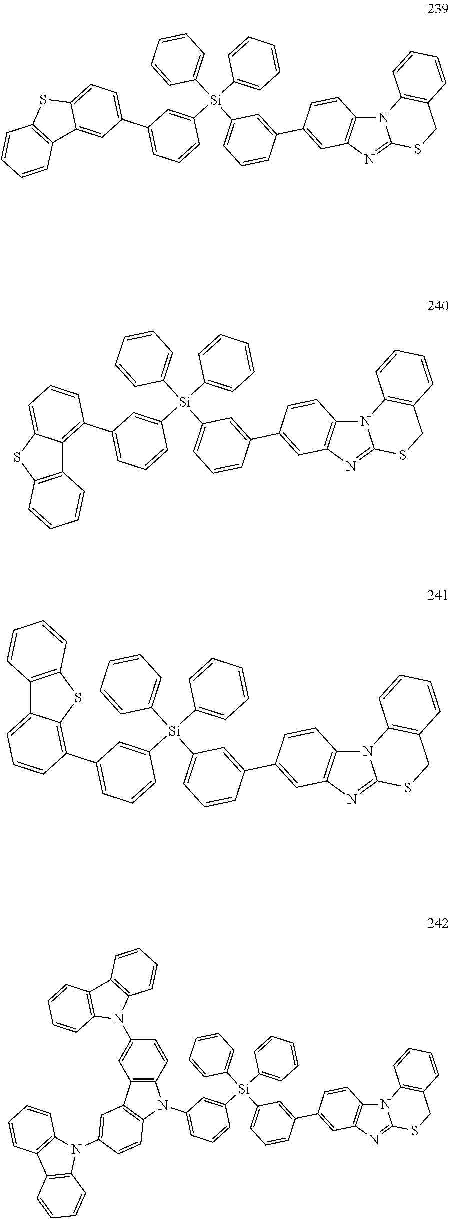 [SMILES: c1ccc([Si](c2ccccc2)(c2cccc(-c3ccc4c(c3)nc3n4-c4ccccc4CS3)c2)c2cccc(-c3ccc4sc5ccccc5c4c3)c2)cc1 ; c1ccc([Si](c2ccccc2)(c2cccc(-c3ccc4c(c3)nc3n4-c4ccccc4CS3)c2)c2cccc(-c3cccc4sc5ccccc5c34)c2)cc1 ; c1ccc([Si](c2ccccc2)(c2cccc(-c3ccc4c(c3)nc3n4-c4ccccc4CS3)c2)c2cccc(-c3cccc4c3sc3ccccc34)c2)cc1 ; c1ccc([Si](c2ccccc2)(c2cccc(-c3ccc4c(c3)nc3n4-c4ccccc4CS3)c2)c2cccc(-n3c4ccc(-n5c6ccccc6c6ccccc65)cc4c4cc(-n5c6ccccc6c6ccccc65)ccc43)c2)cc1]

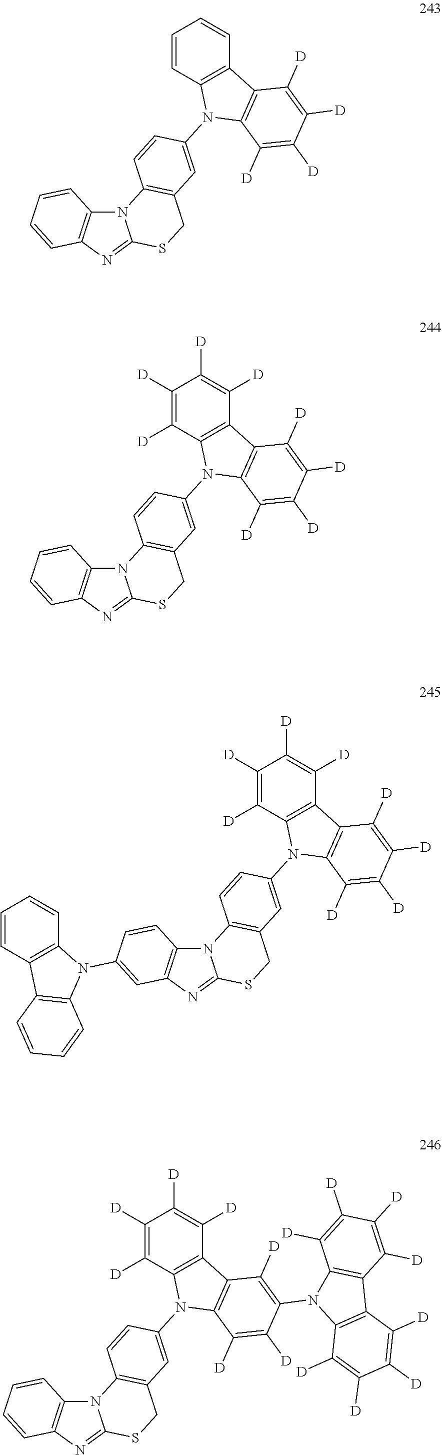 [SMILES: [2H]c1c([2H])c([2H])c2c(c1[2H])c1ccccc1n2-c1ccc2c(c1)CSc1nc3ccccc3n1-2 ; [2H]c1c([2H])c([2H])c2c(c1[2H])c1c([2H])c([2H])c([2H])c([2H])c1n2-c1ccc2c(c1)CSc1nc3ccccc3n1-2 ; [2H]c1c([2H])c([2H])c2c(c1[2H])c1c([2H])c([2H])c([2H])c([2H])c1n2-c1ccc2c(c1)CSc1nc3cc(-n4c5ccccc5c5ccccc54)ccc3n1-2 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc2c(c1)CSc1nc3ccccc3n1-2]

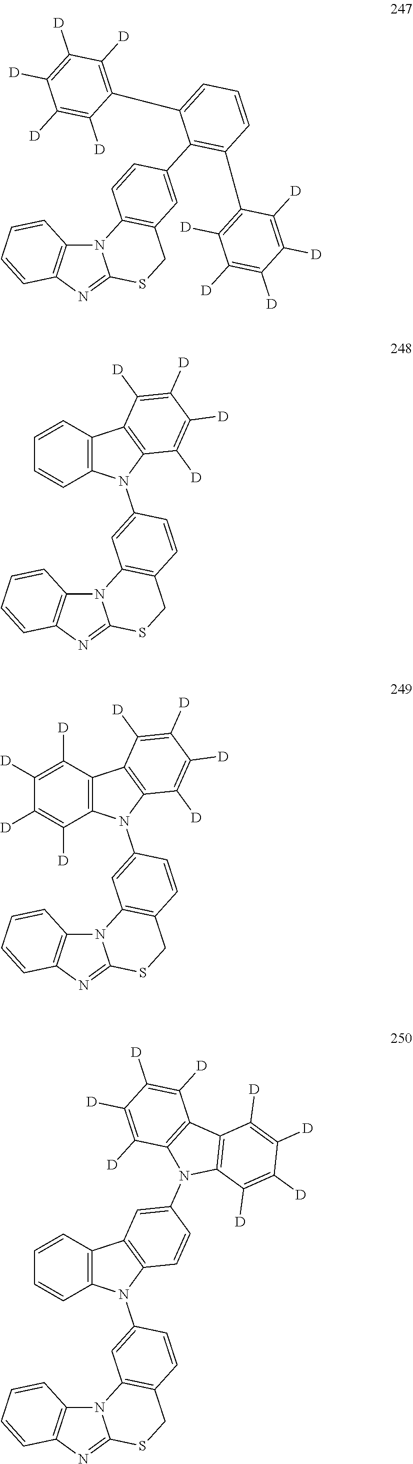 [SMILES: [2H]c1c([2H])c([2H])c(-c2cccc(-c3c([2H])c([2H])c([2H])c([2H])c3[2H])c2-c2ccc3c(c2)CSc2nc4ccccc4n2-3)c([2H])c1[2H] ; [2H]c1c([2H])c([2H])c2c(c1[2H])c1ccccc1n2-c1ccc2c(c1)-n1c(nc3ccccc31)SC2 ; [2H]c1c([2H])c([2H])c2c(c1[2H])c1c([2H])c([2H])c([2H])c([2H])c1n2-c1ccc2c(c1)-n1c(nc3ccccc31)SC2 ; [2H]c1c([2H])c([2H])c2c(c1[2H])c1c([2H])c([2H])c([2H])c([2H])c1n2-c1ccc2c(c1)c1ccccc1n2-c1ccc2c(c1)-n1c(nc3ccccc31)SC2]

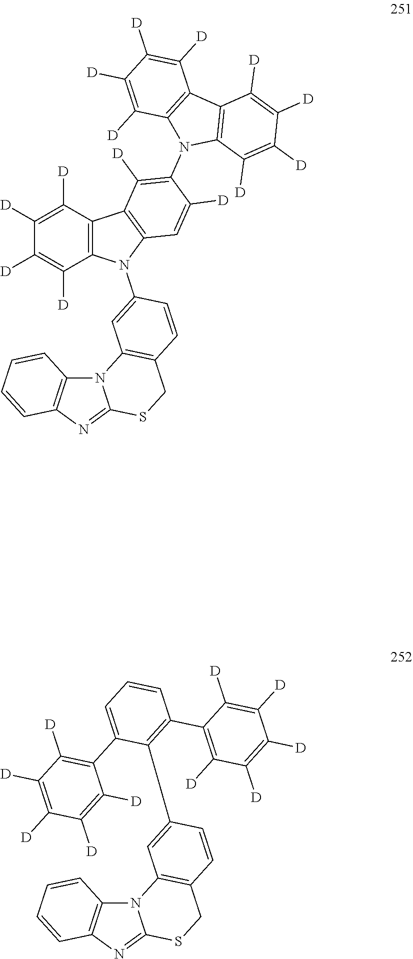 [SMILES: [2H]c1cc2c(c([2H])c1-n1c3c([2H])c([2H])c([2H])c([2H])c3c3c([2H])c([2H])c([2H])c([2H])c31)c1c([2H])c([2H])c([2H])c([2H])c1n2-c1ccc2c(c1)-n1c(nc3ccccc31)SC2 ; [2H]c1c([2H])c([2H])c(-c2cccc(-c3c([2H])c([2H])c([2H])c([2H])c3[2H])c2-c2ccc3c(c2)-n2c(nc4ccccc42)SC3)c([2H])c1[2H]]

Group II

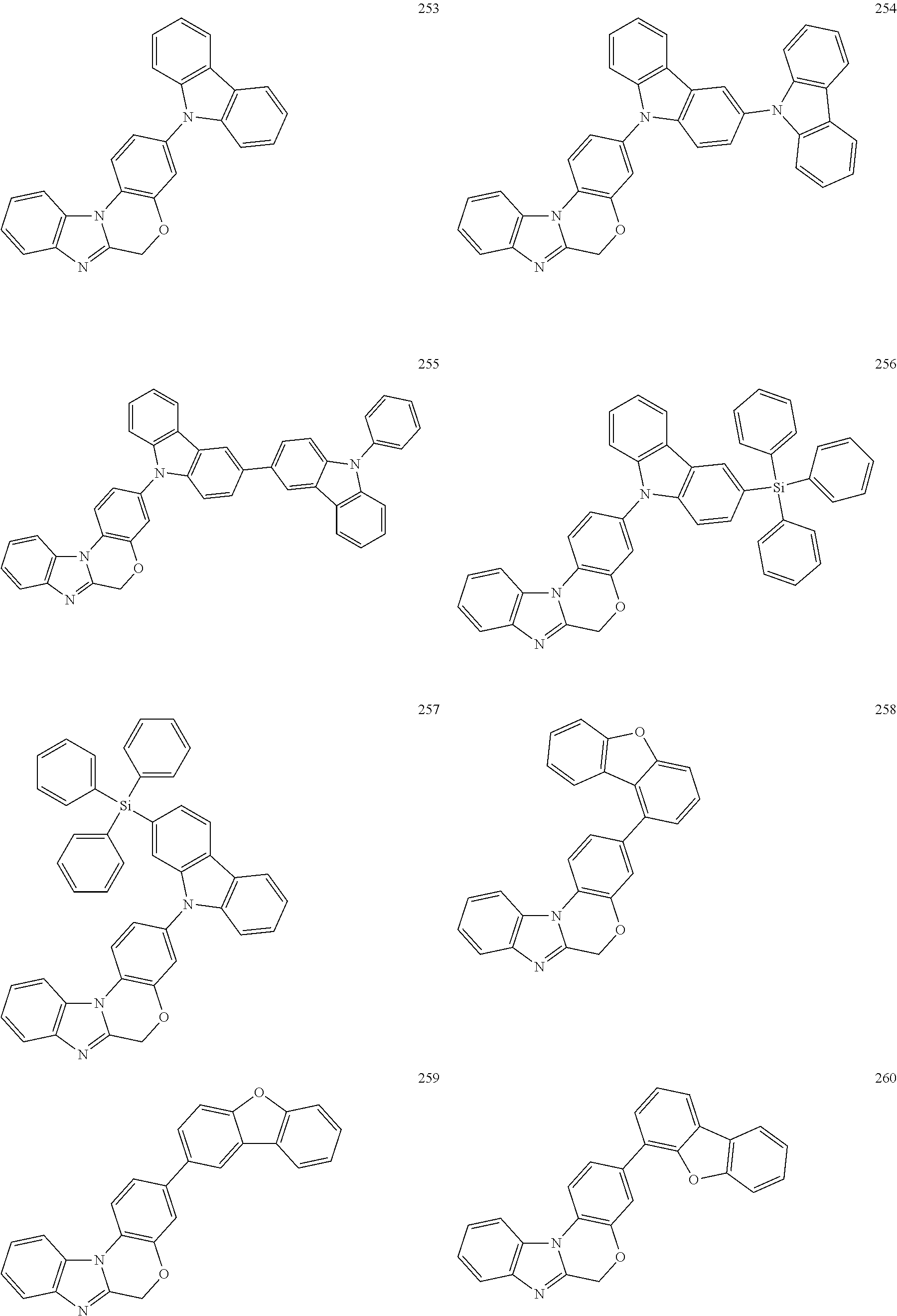

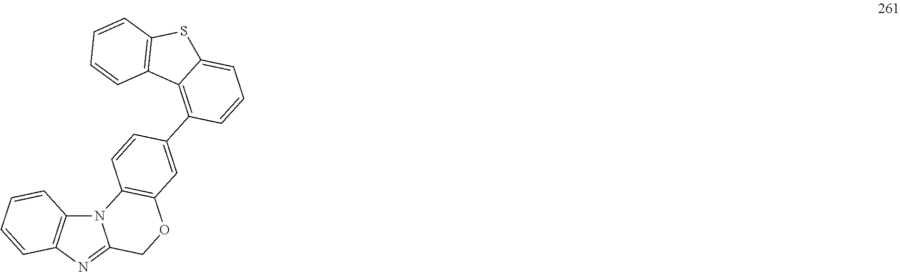 [SMILES: c1ccc2c(c1)nc1n2-c2ccc(-c3cccc4sc5ccccc5c34)cc2OC1]

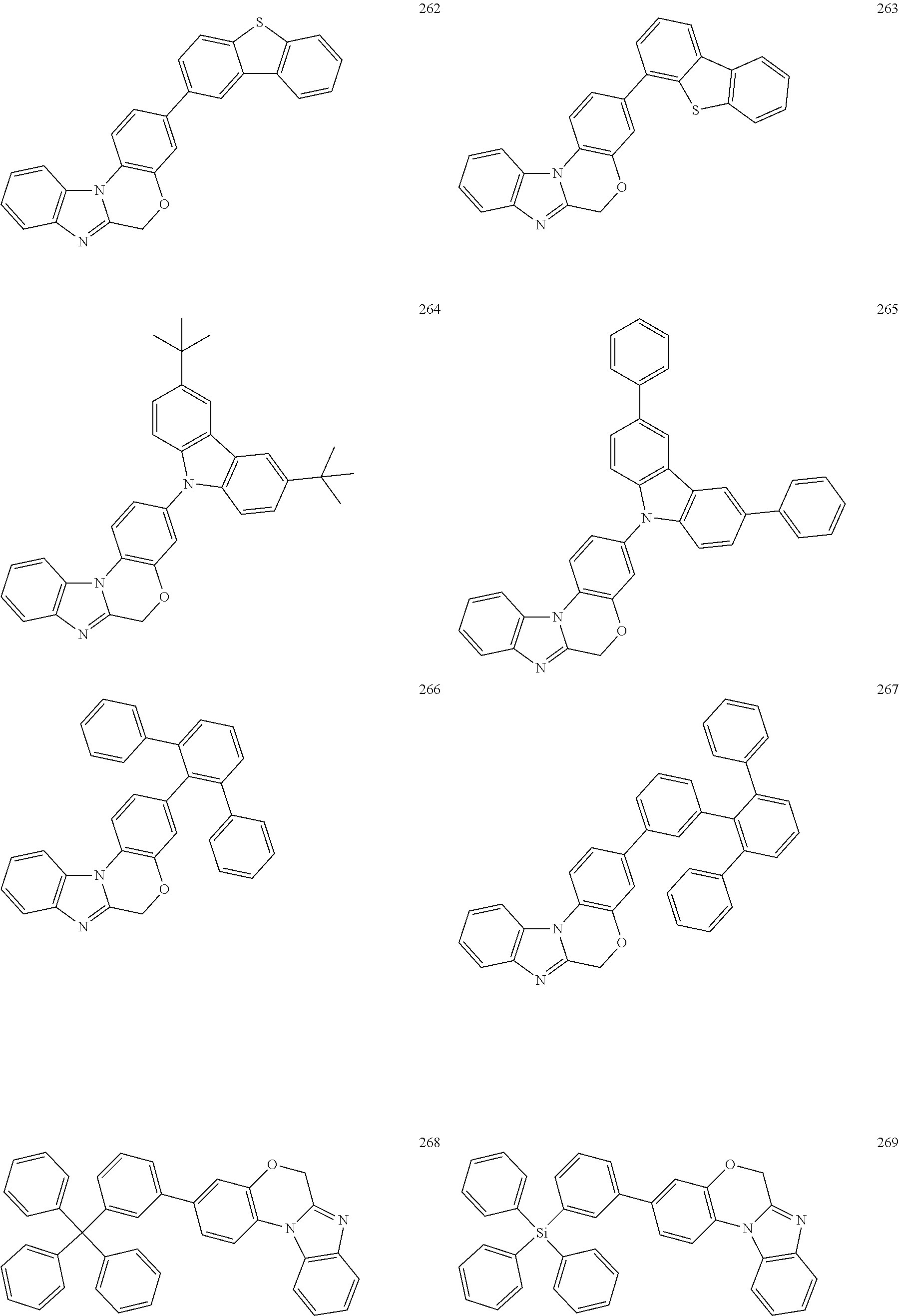

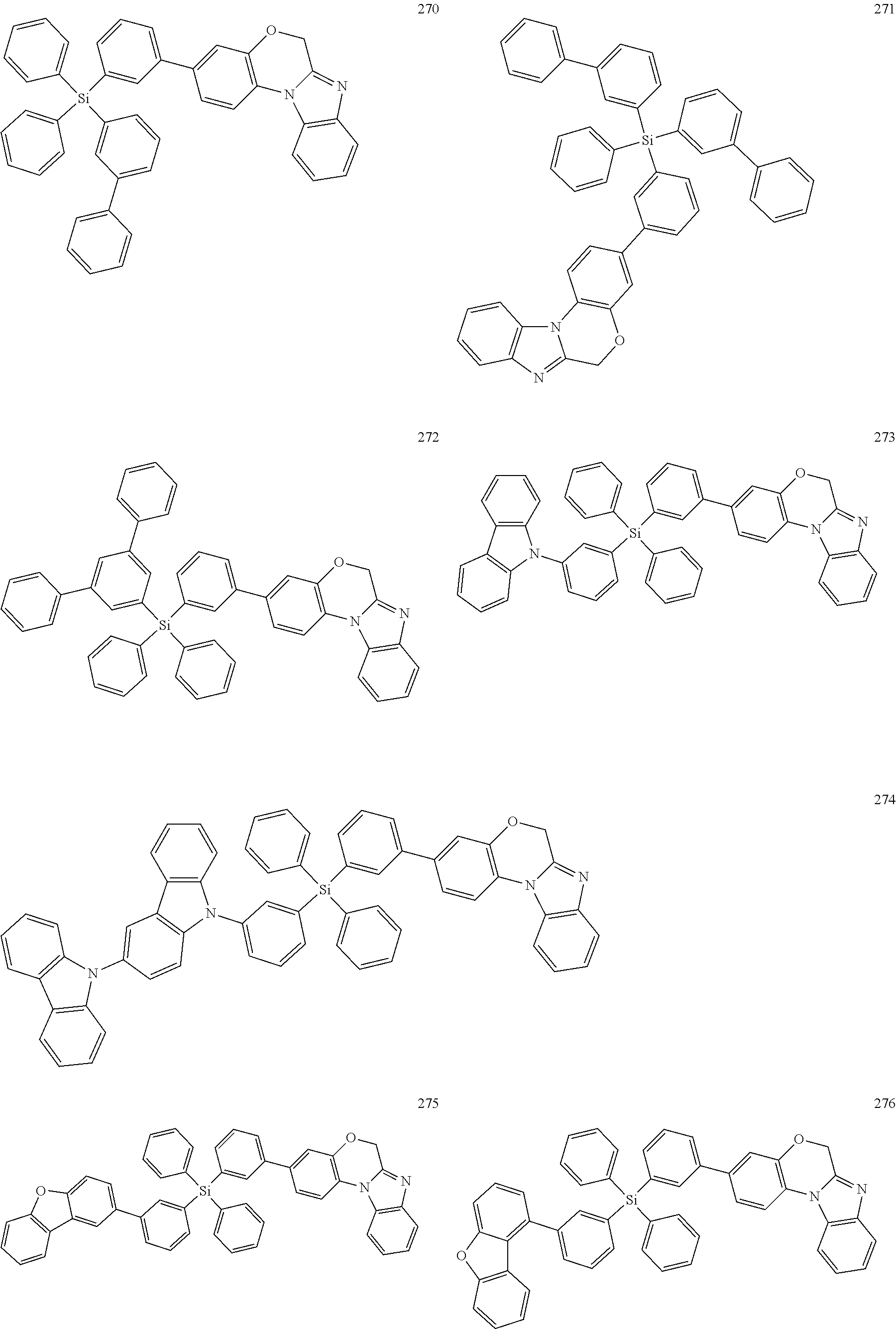

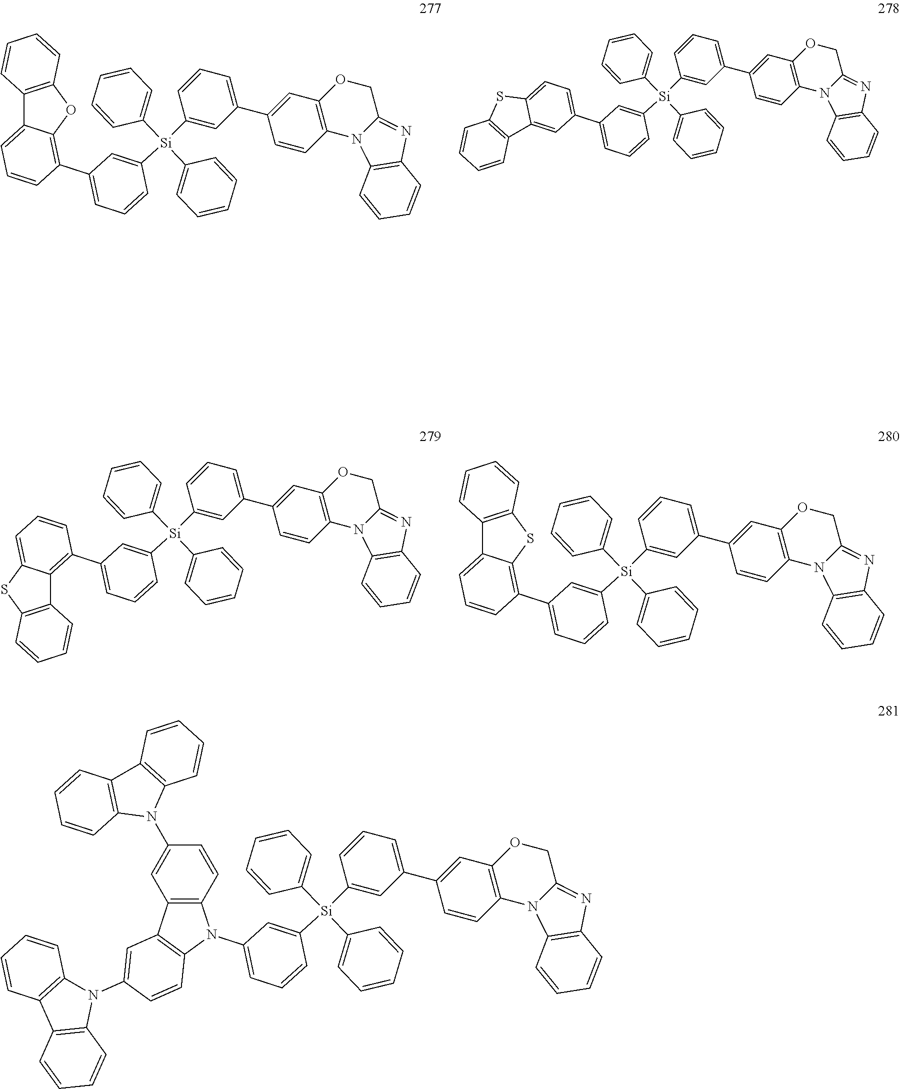

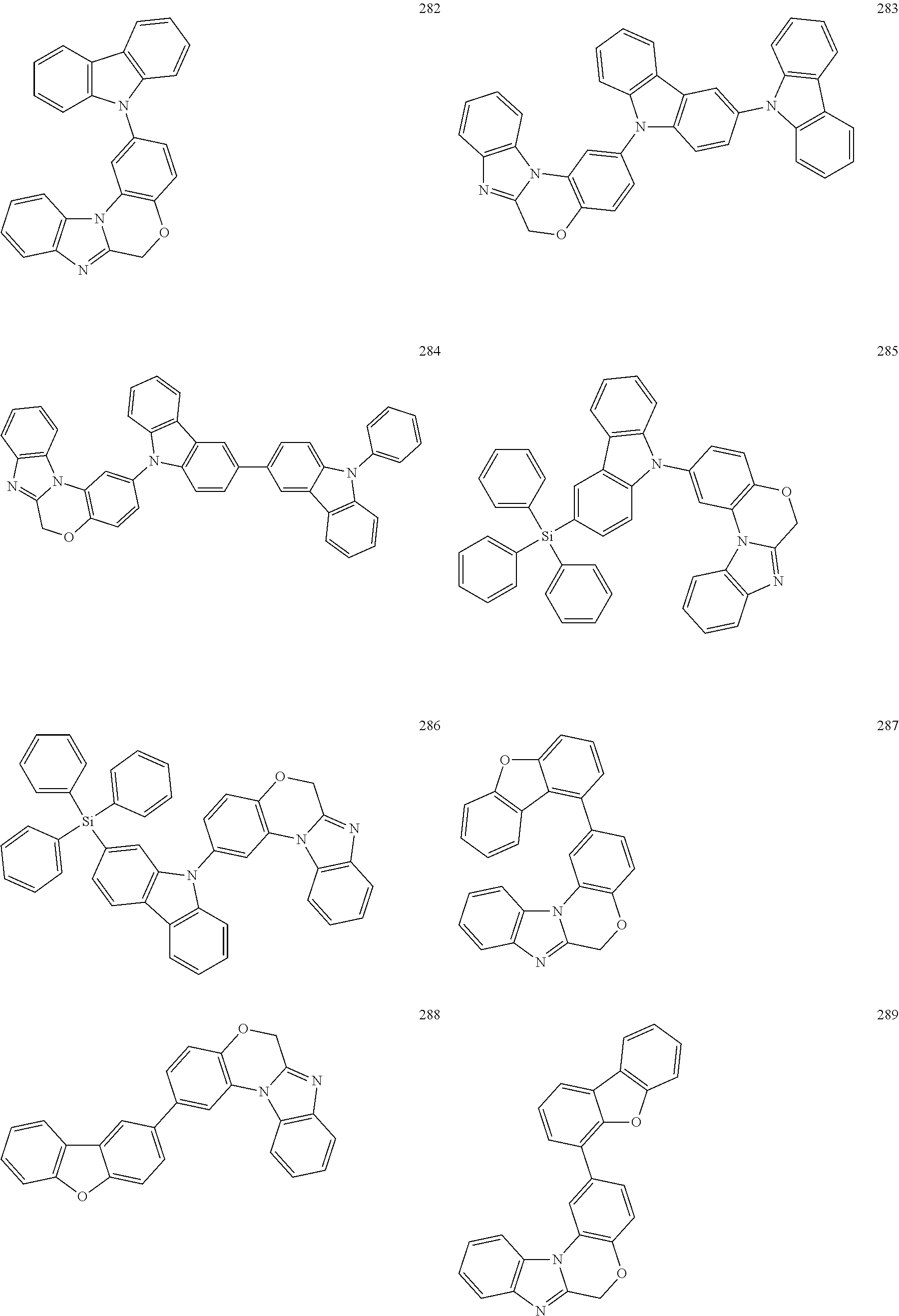

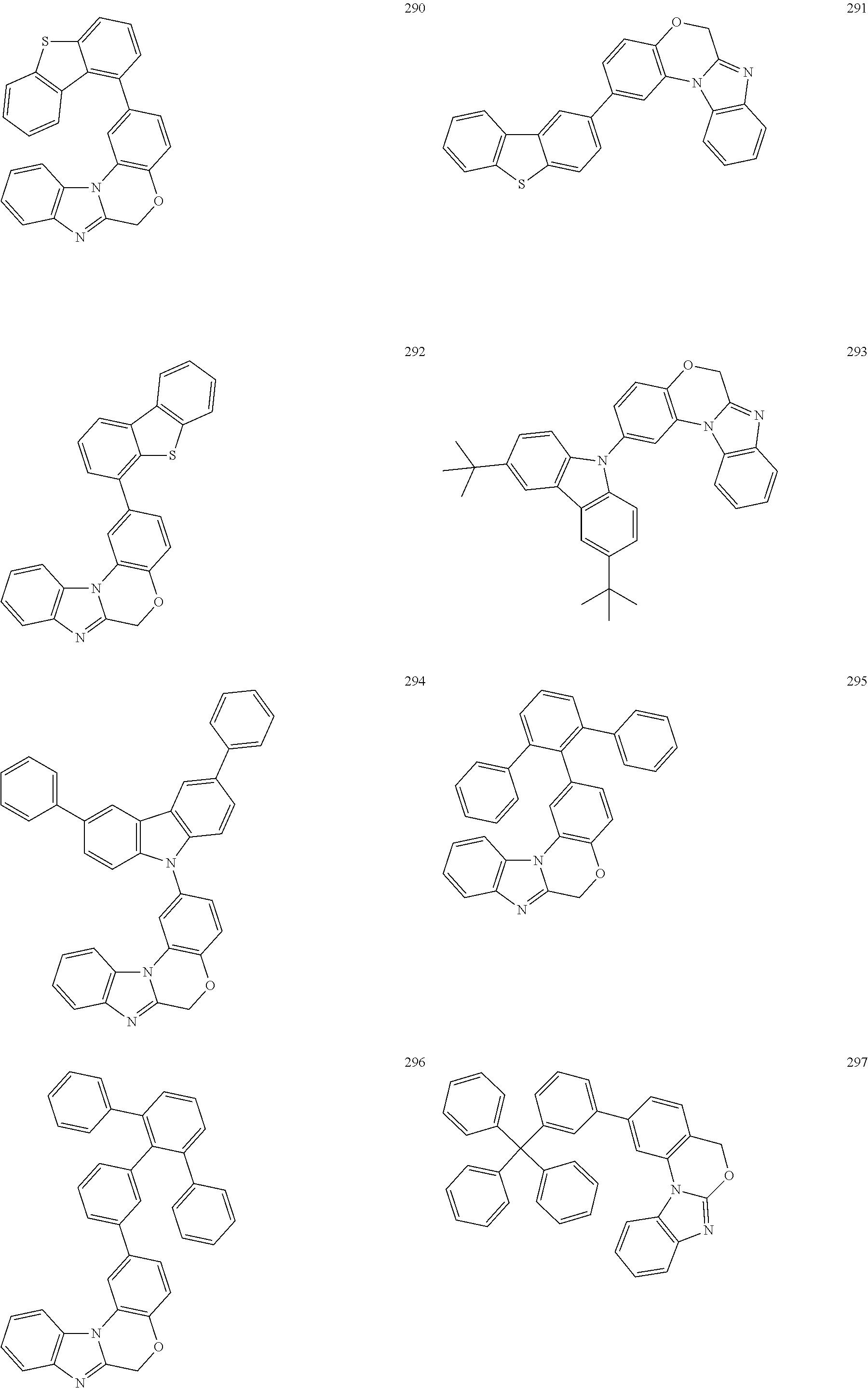

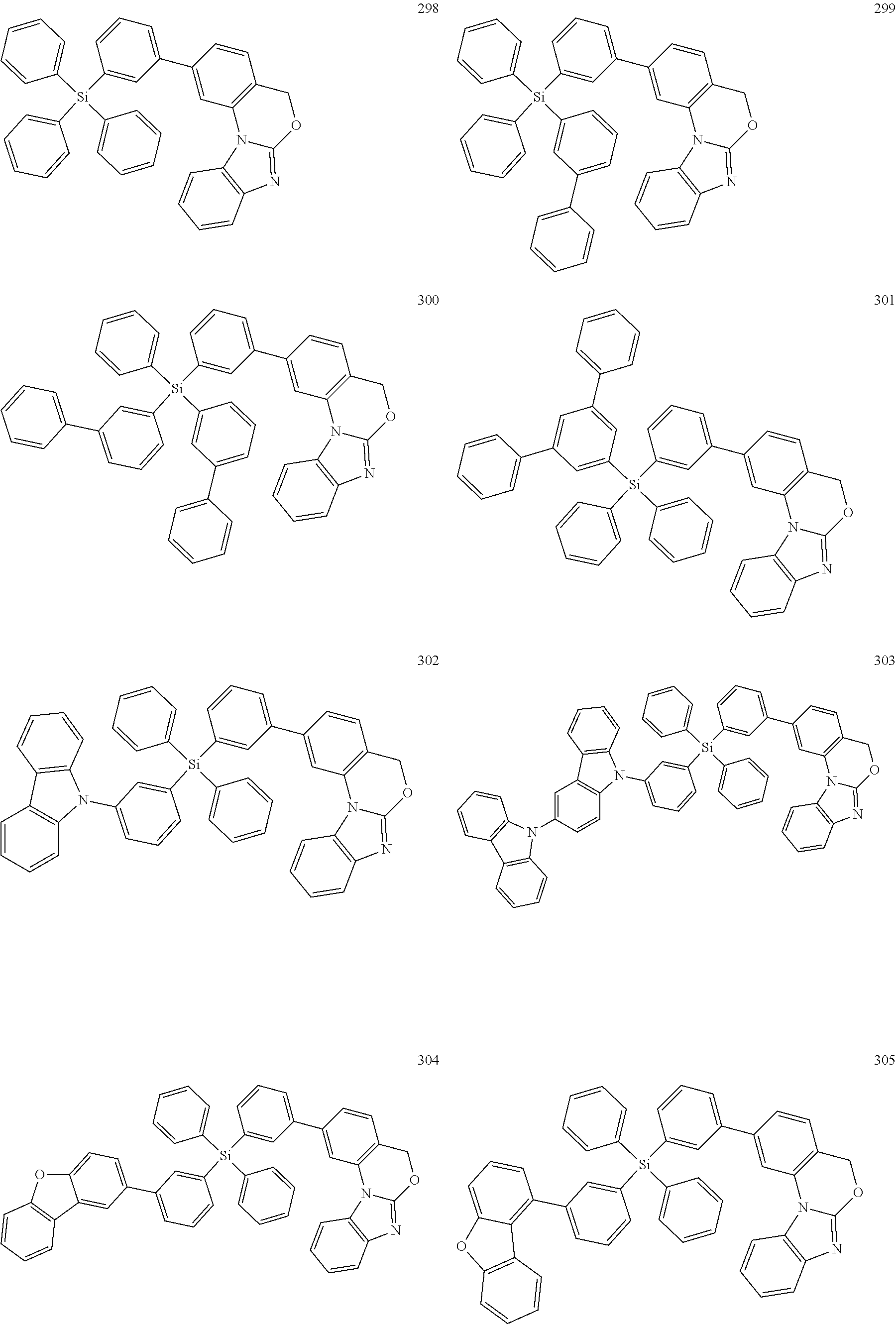

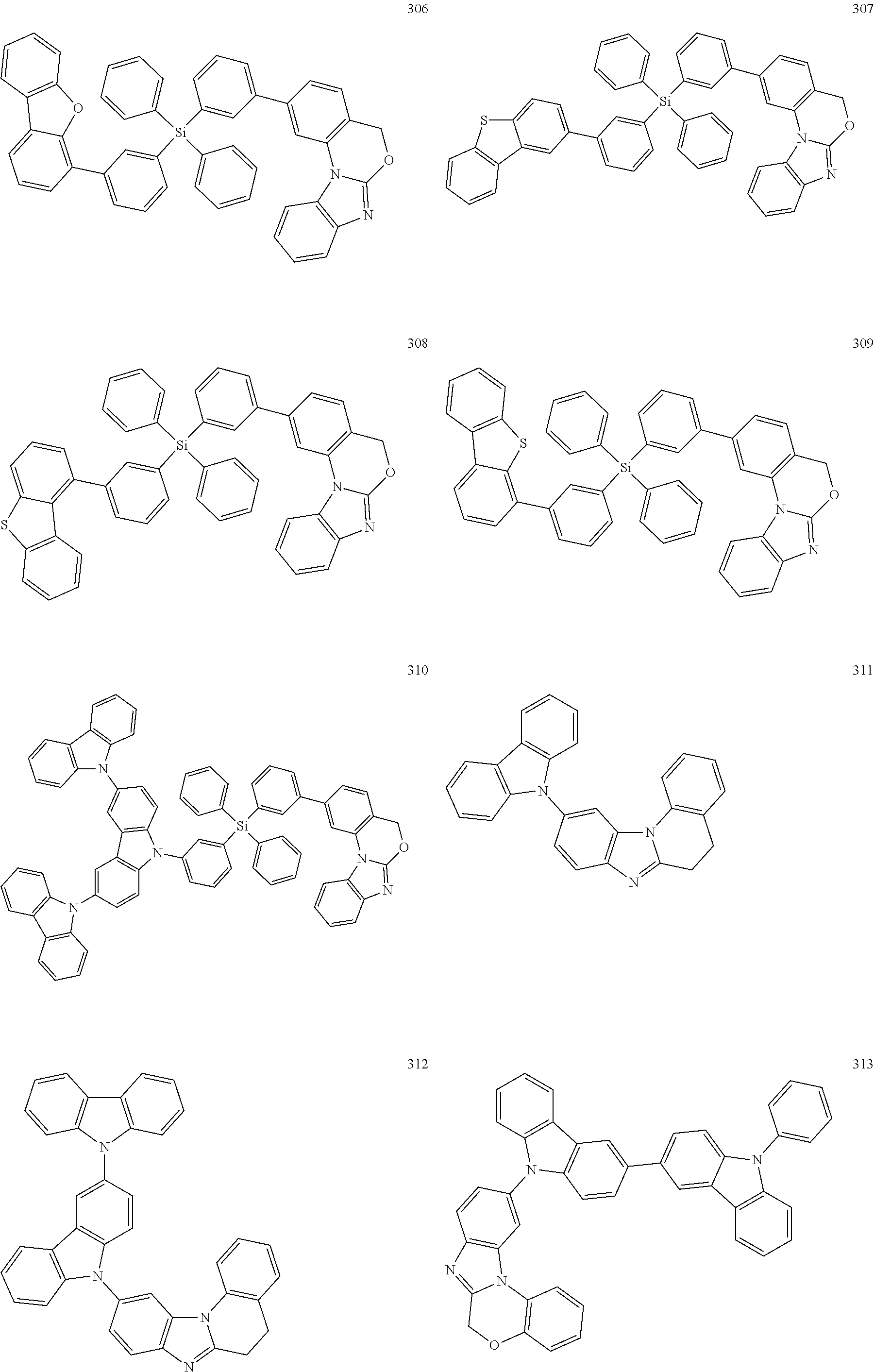

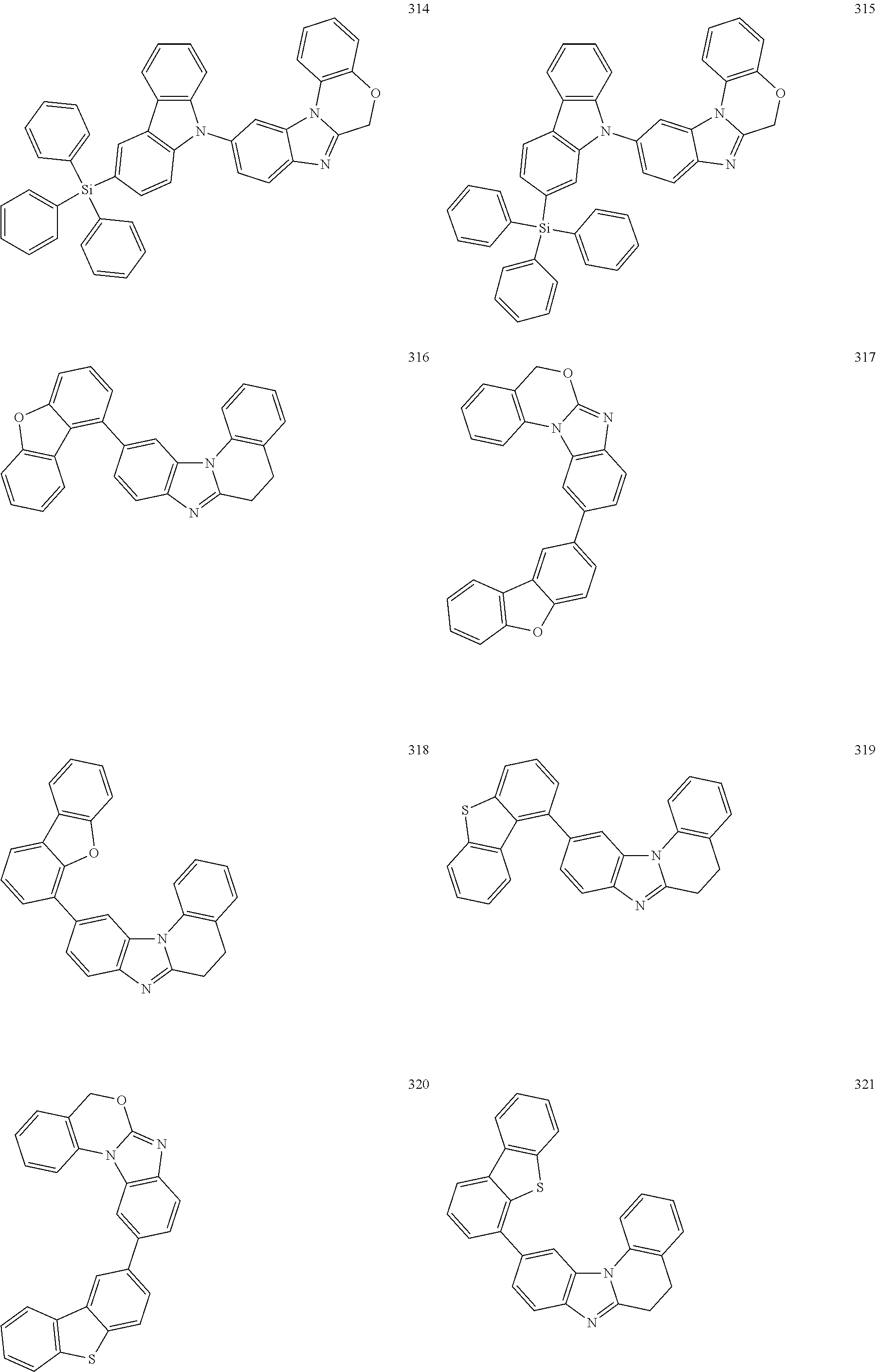

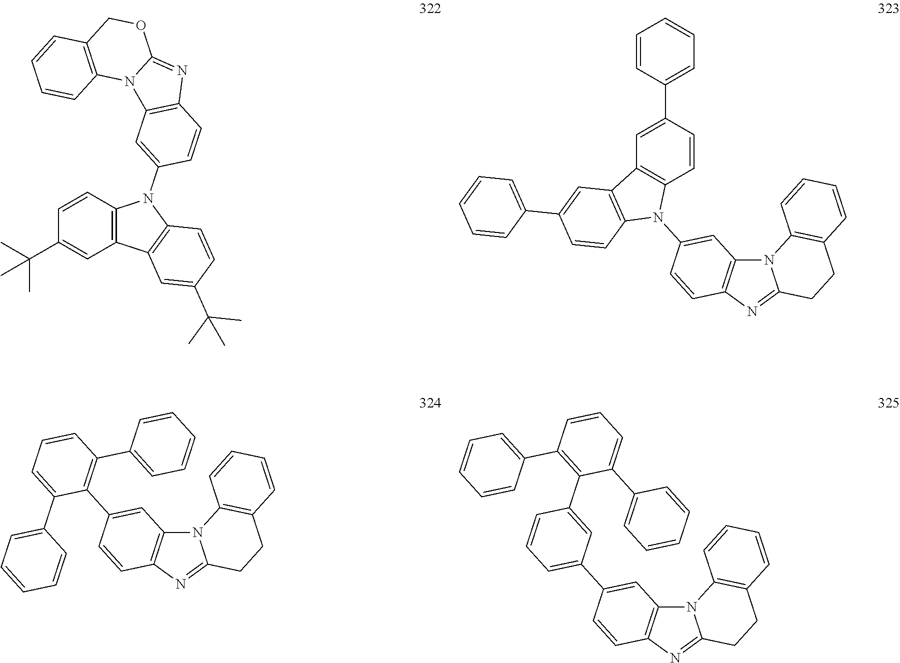 [SMILES: CC(C)(C)c1ccc2c(c1)c1cc(C(C)(C)C)ccc1n2-c1ccc2nc3n(c2c1)-c1ccccc1CO3 ; c1ccc(-c2ccc3c(c2)c2cc(-c4ccccc4)ccc2n3-c2ccc3nc4n(c3c2)-c2ccccc2CC4)cc1 ; c1ccc(-c2cccc(-c3ccccc3)c2-c2ccc3nc4n(c3c2)-c2ccccc2CC4)cc1 ; c1ccc(-c2cccc(-c3ccccc3)c2-c2cccc(-c3ccc4nc5n(c4c3)-c3ccccc3CC5)c2)cc1]

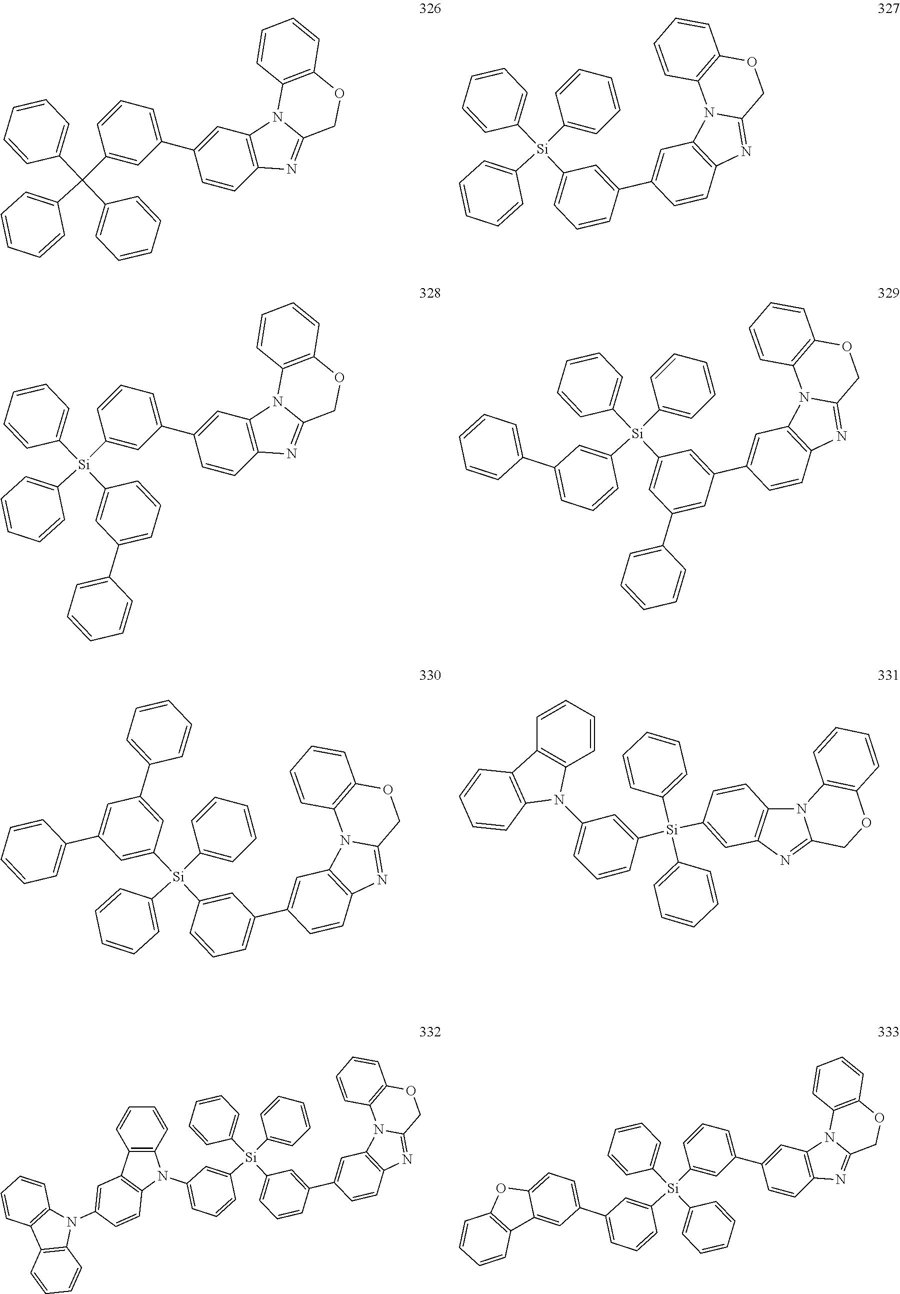

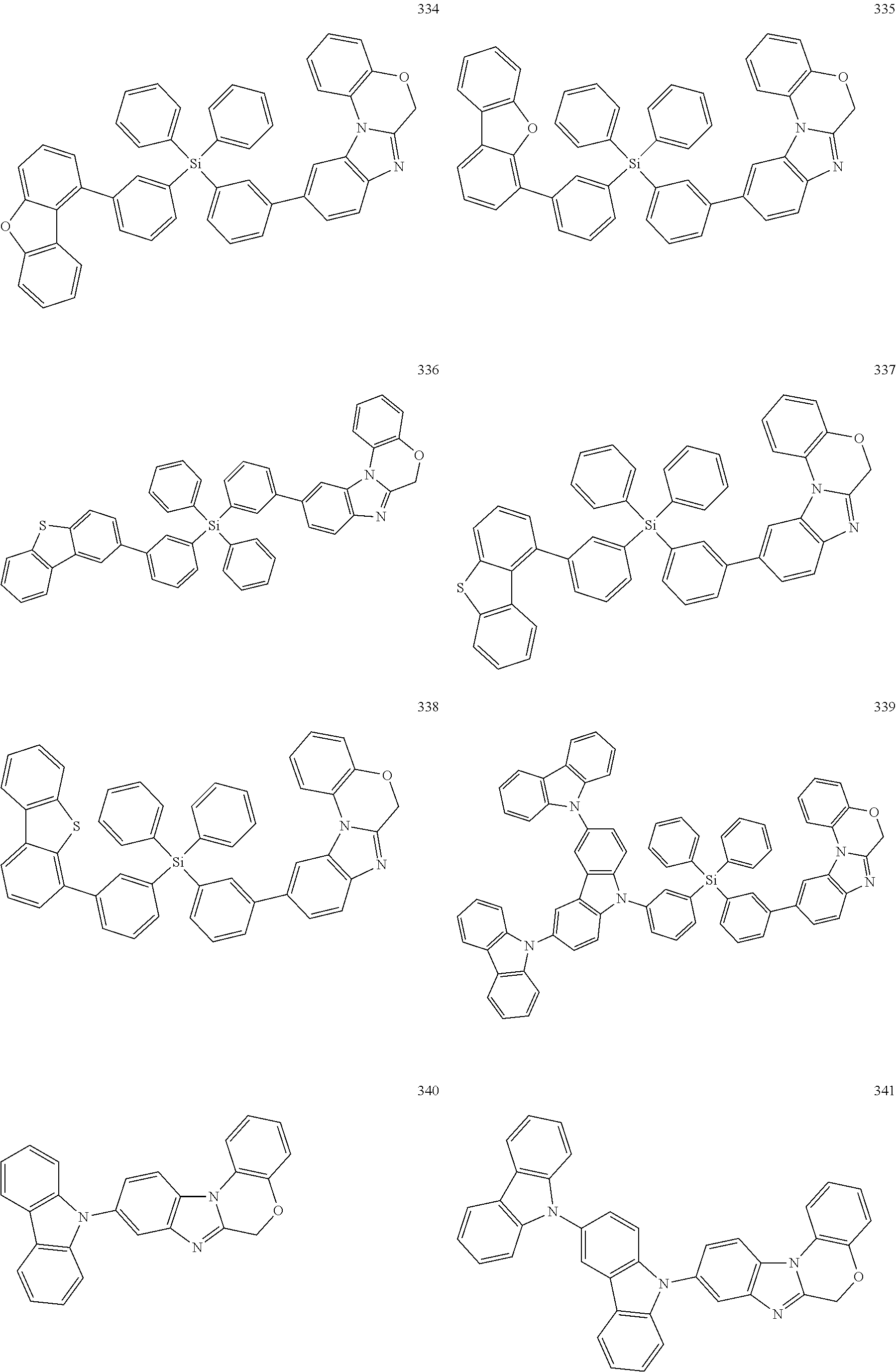

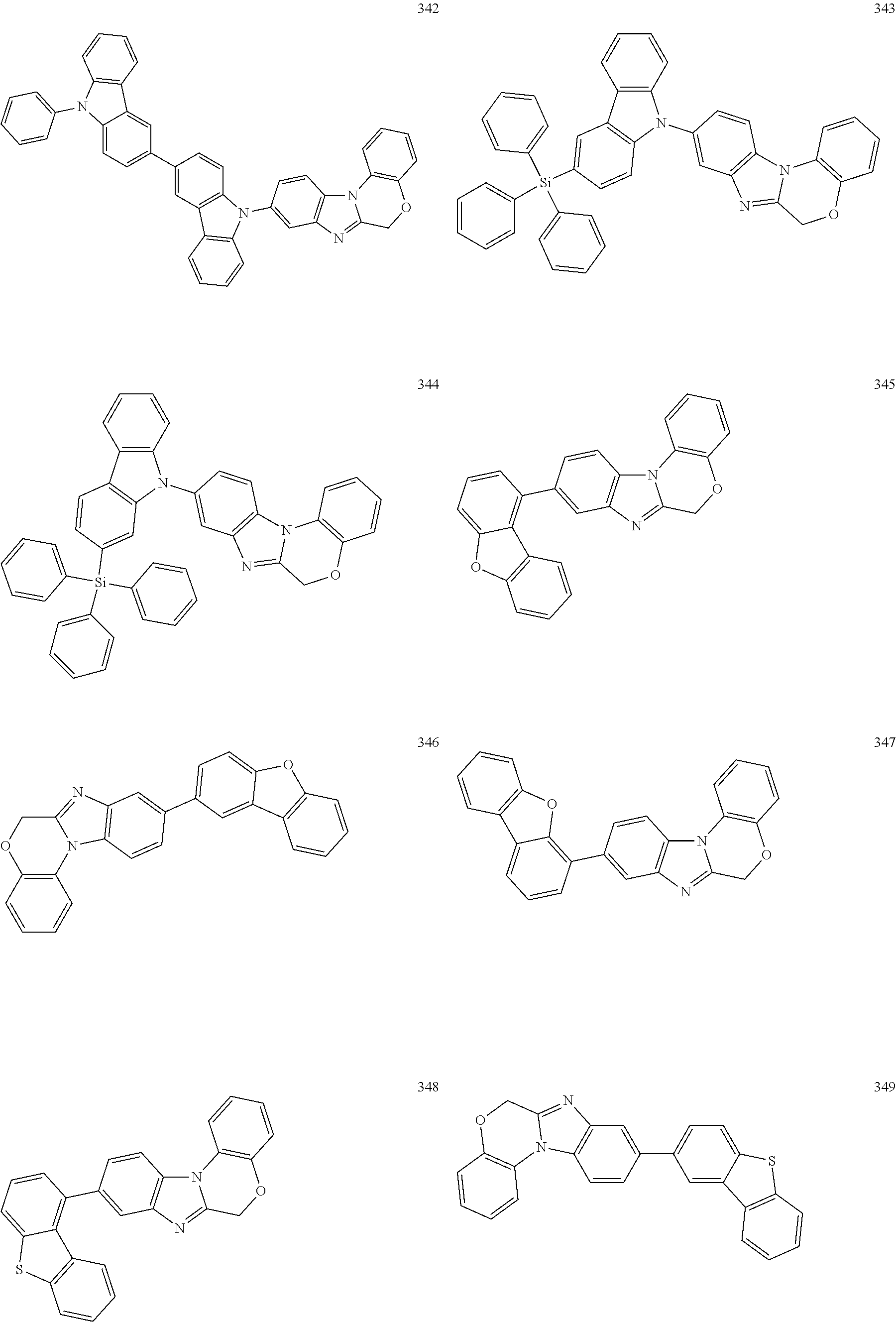

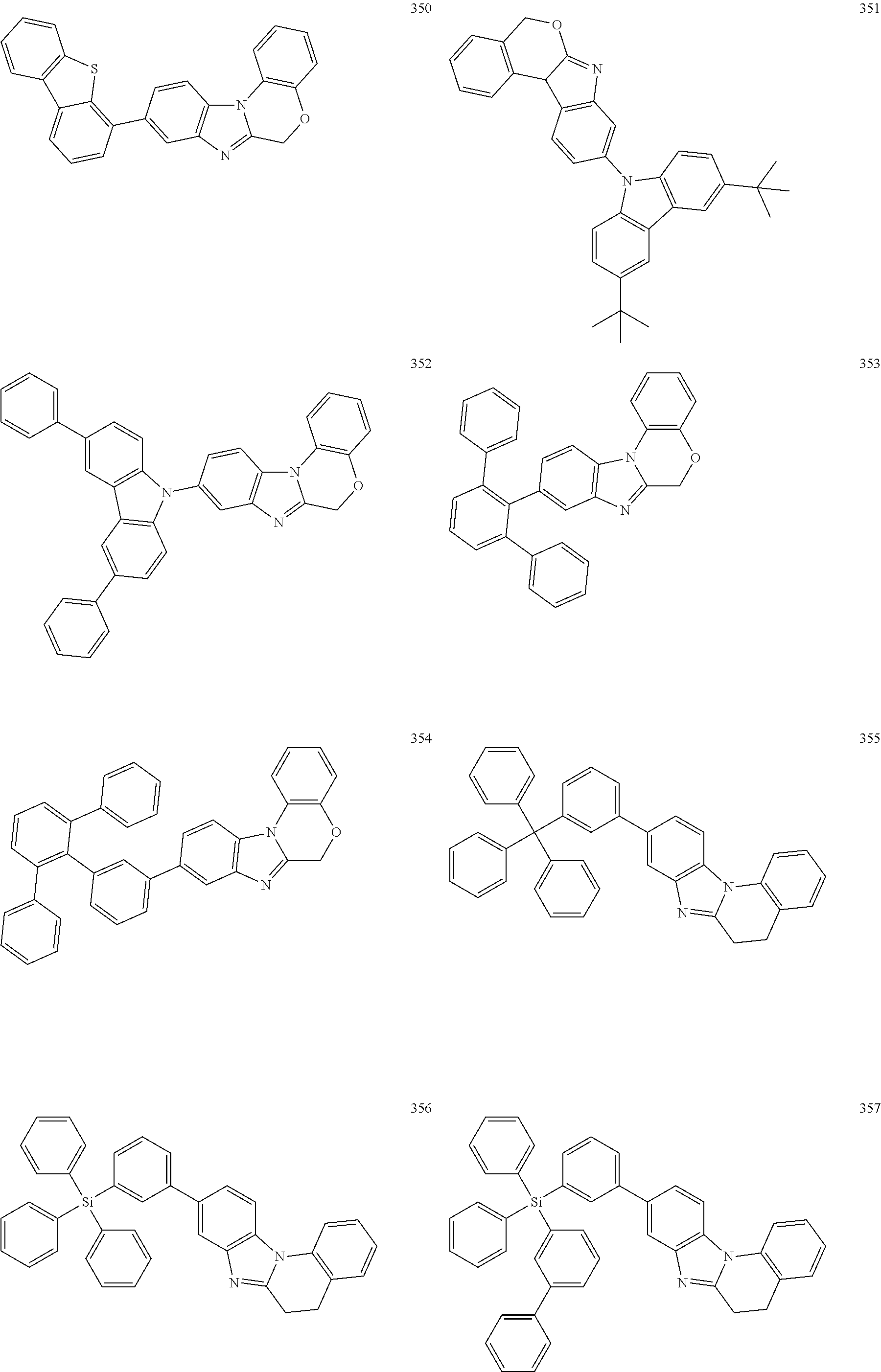

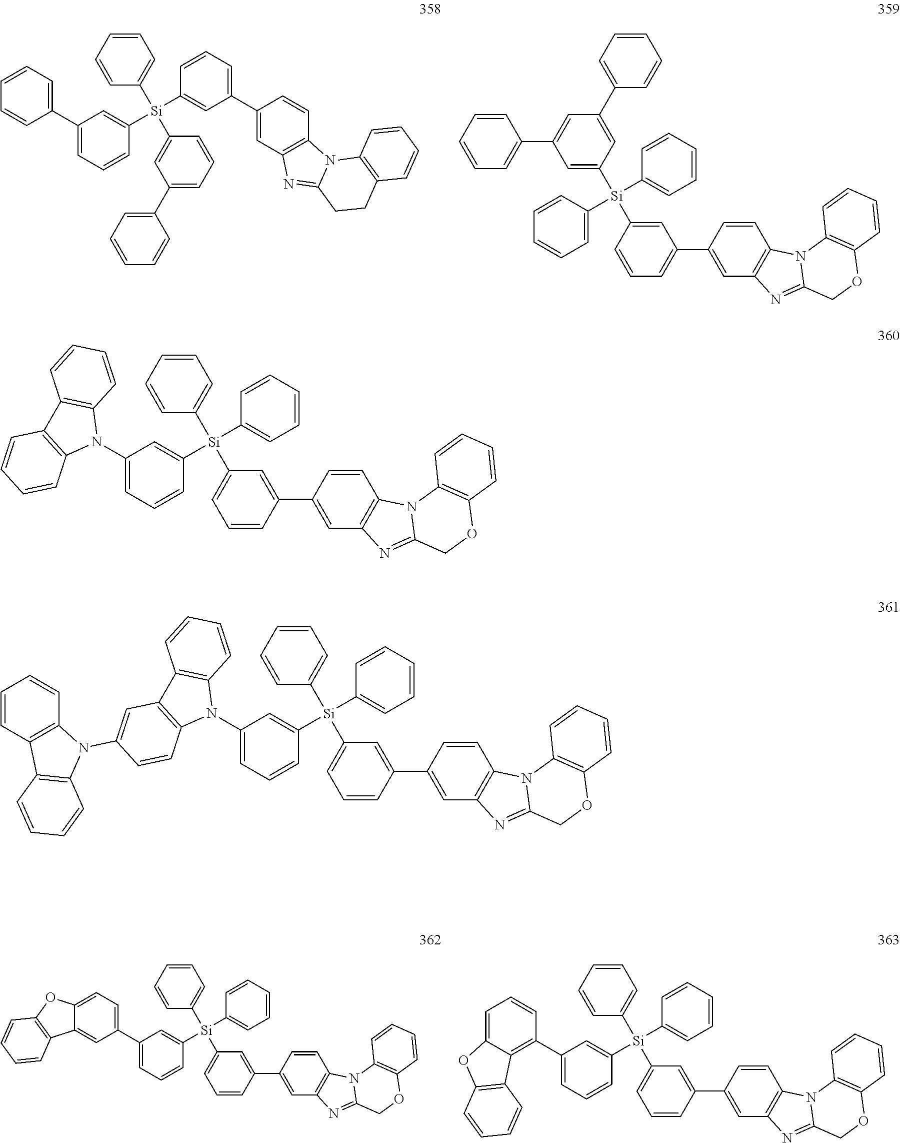

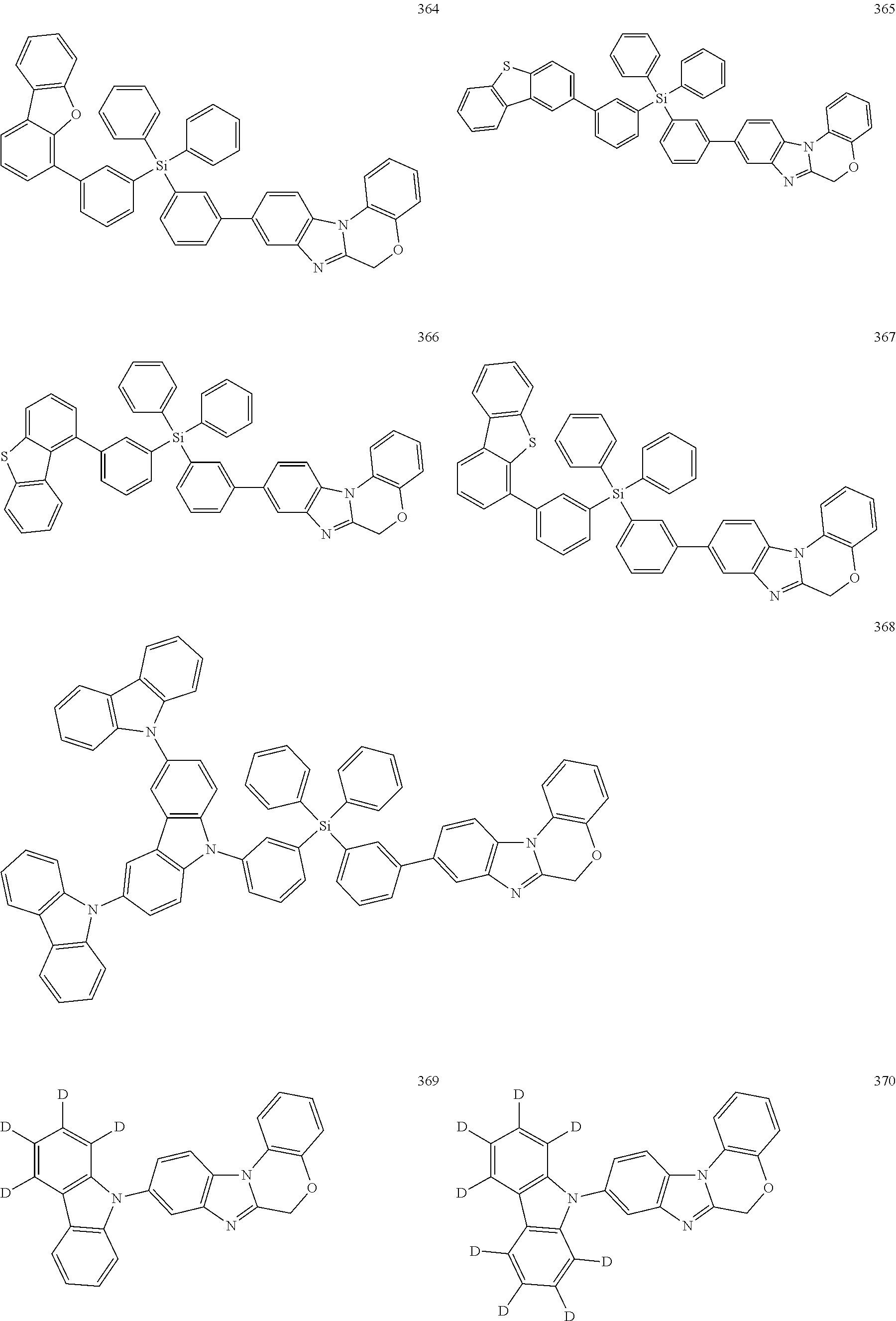

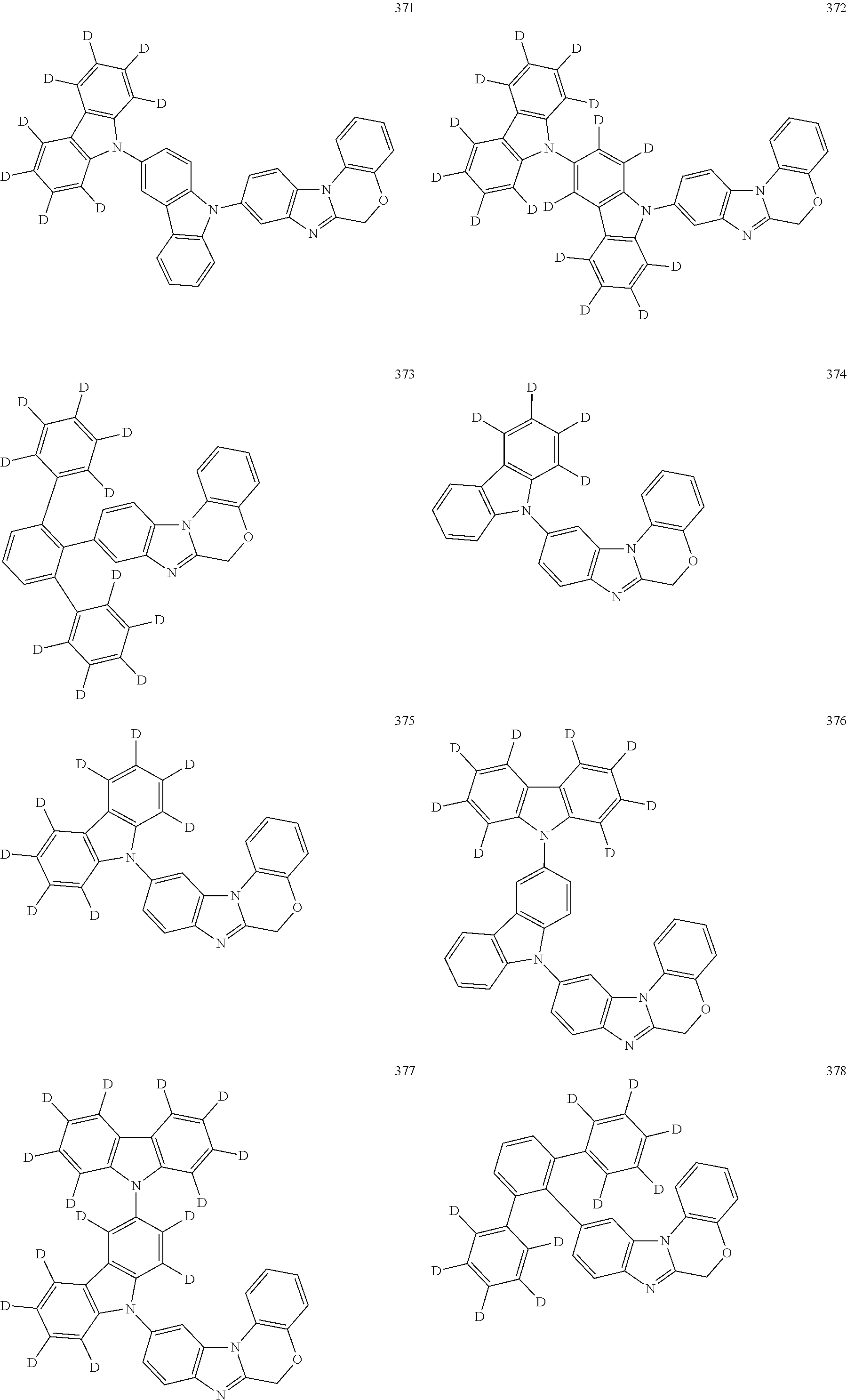

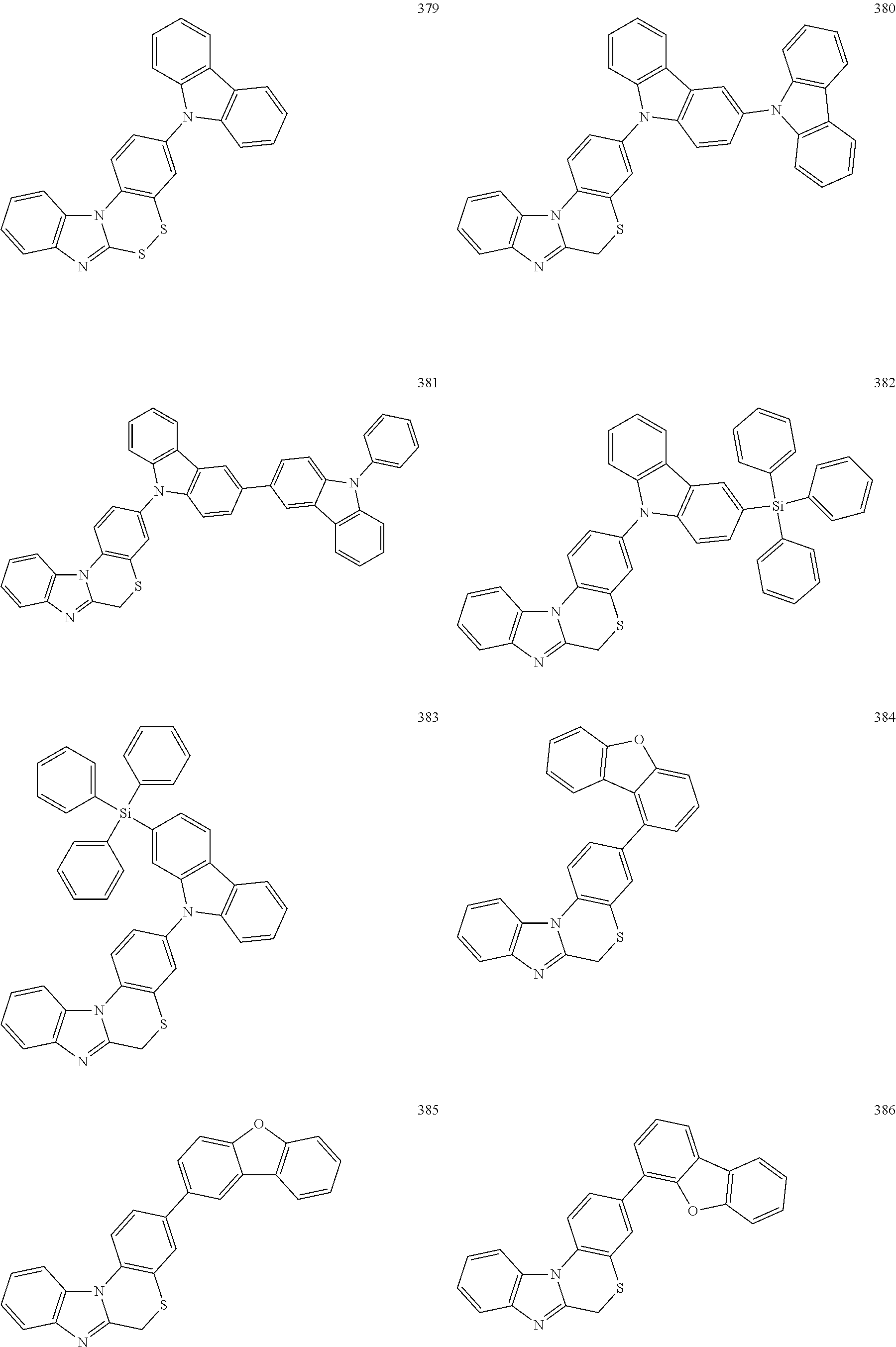

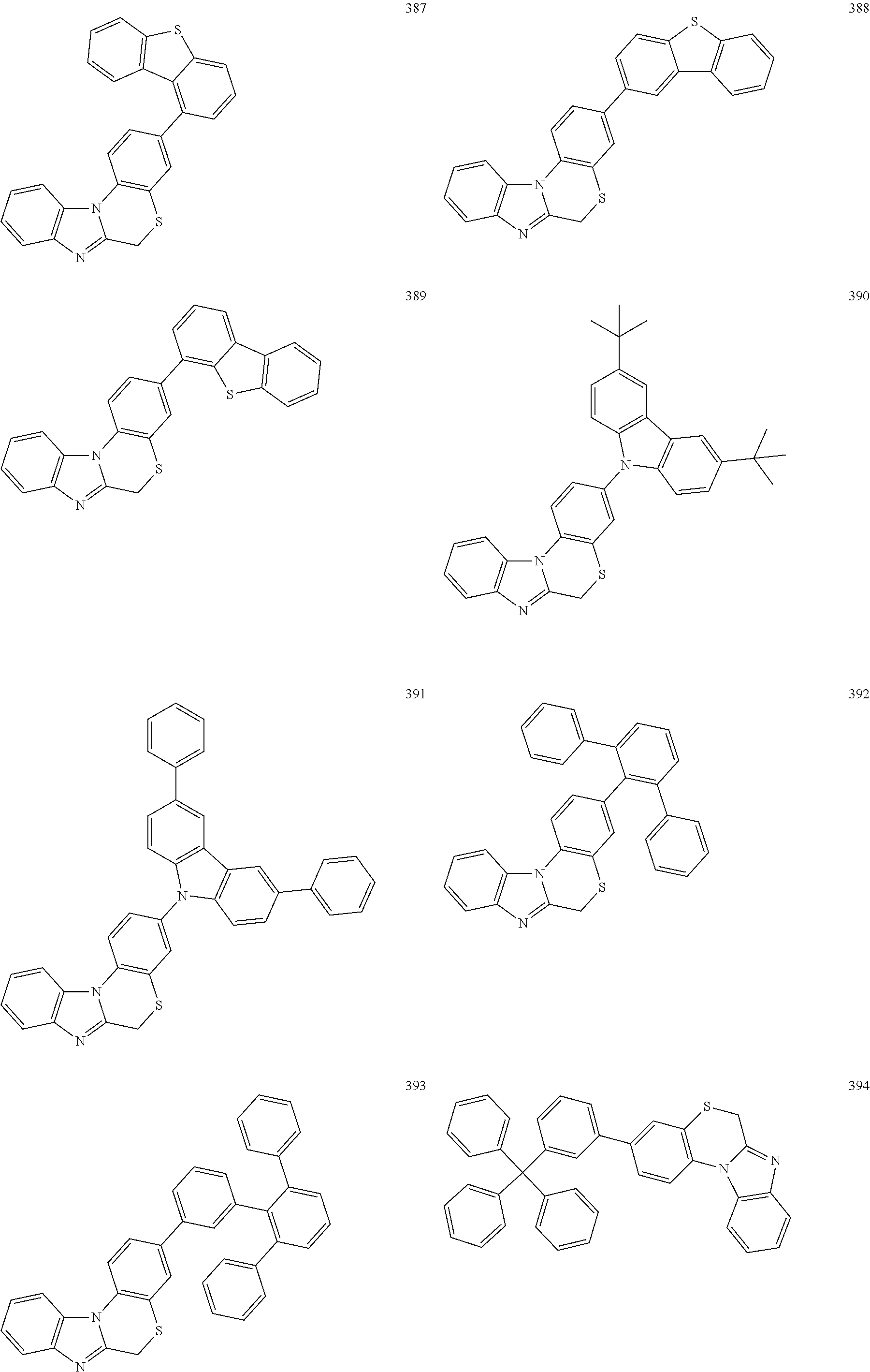

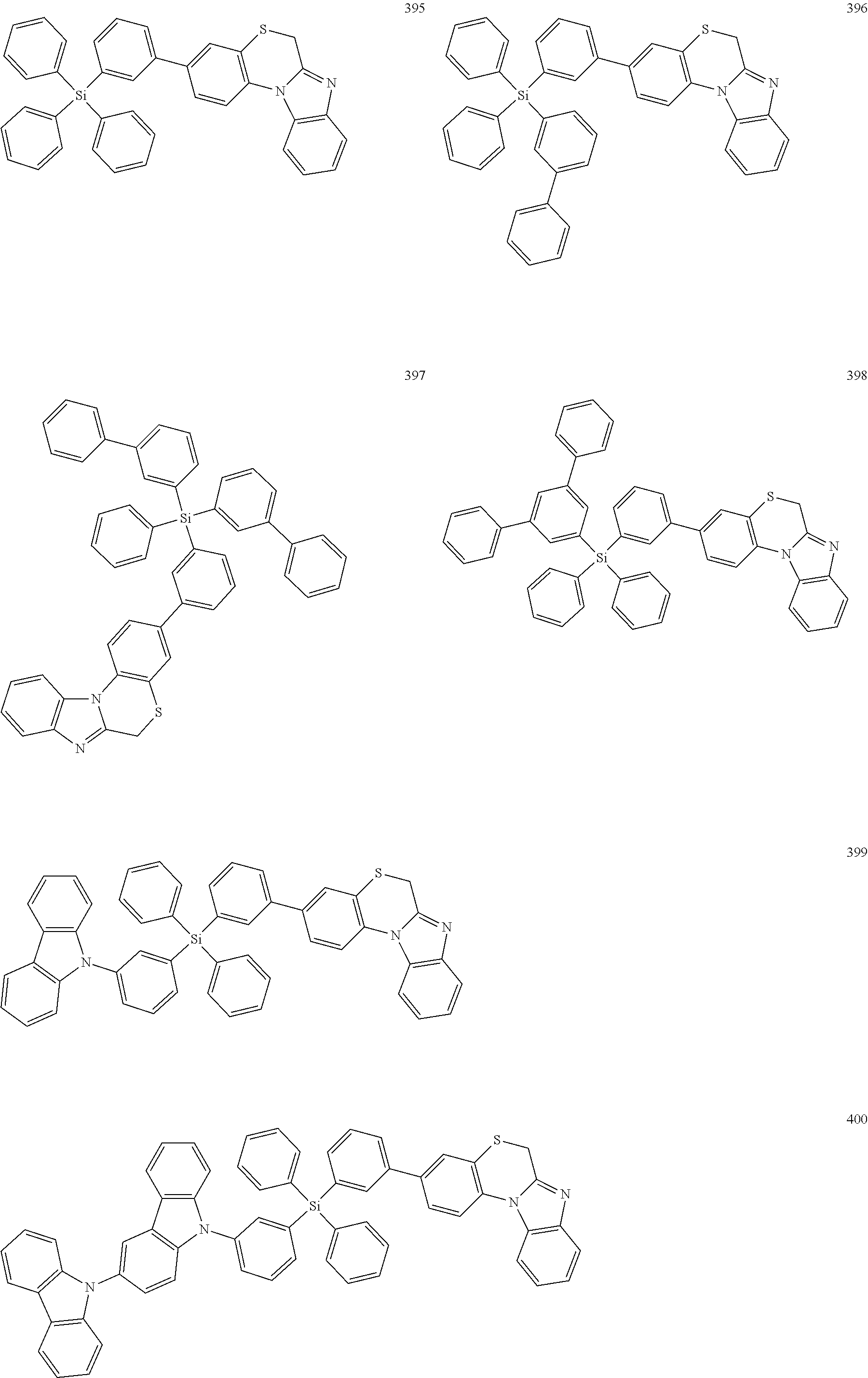

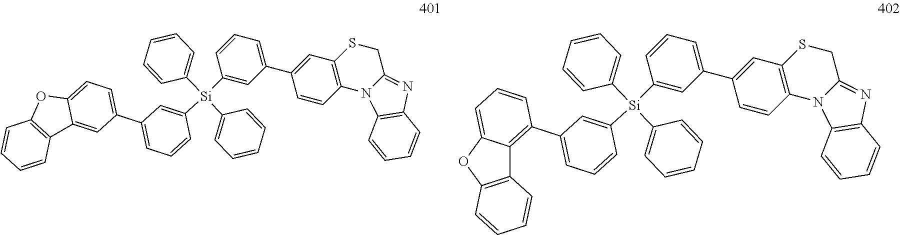 [SMILES: c1ccc([Si](c2ccccc2)(c2cccc(-c3ccc4c(c3)SCc3nc5ccccc5n3-4)c2)c2cccc(-c3ccc4oc5ccccc5c4c3)c2)cc1 ; c1ccc([Si](c2ccccc2)(c2cccc(-c3ccc4c(c3)SCc3nc5ccccc5n3-4)c2)c2cccc(-c3cccc4oc5ccccc5c34)c2)cc1]

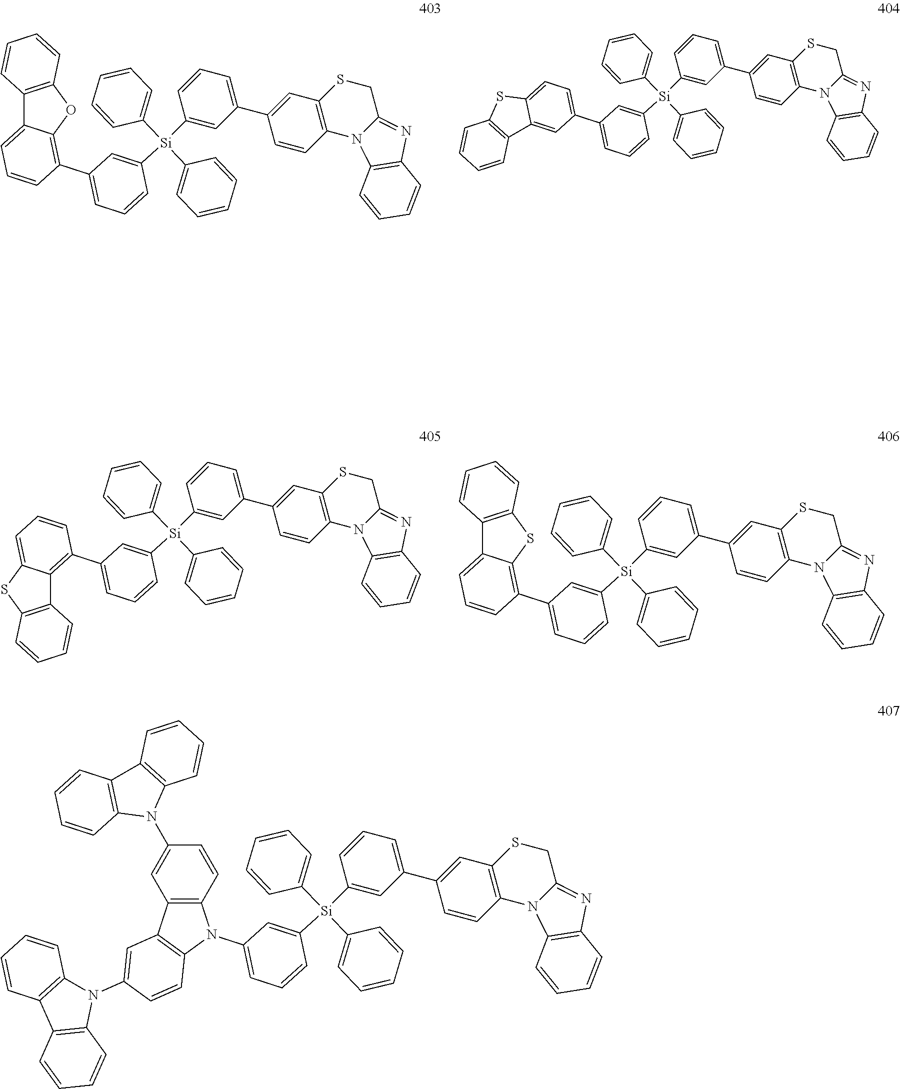

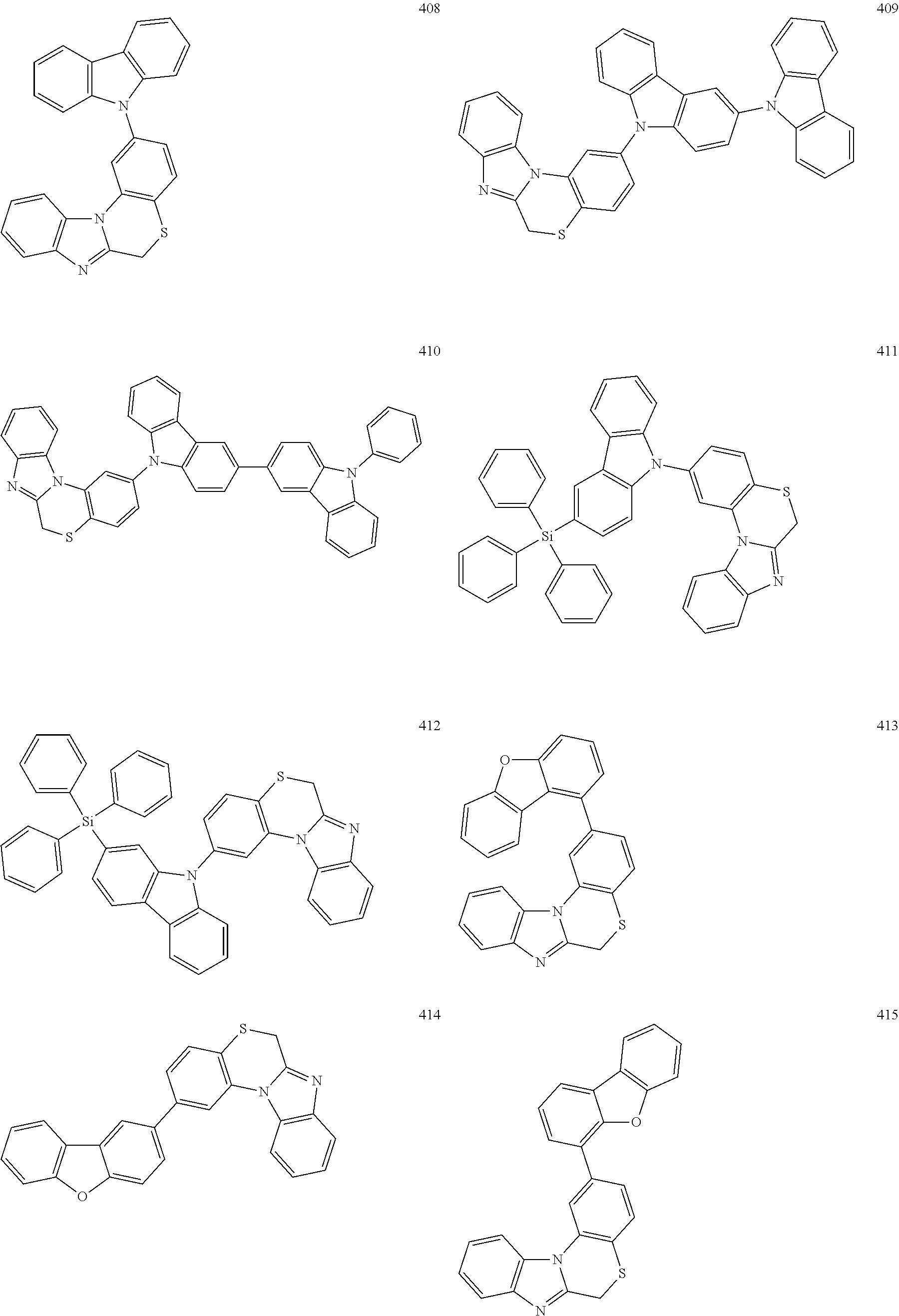

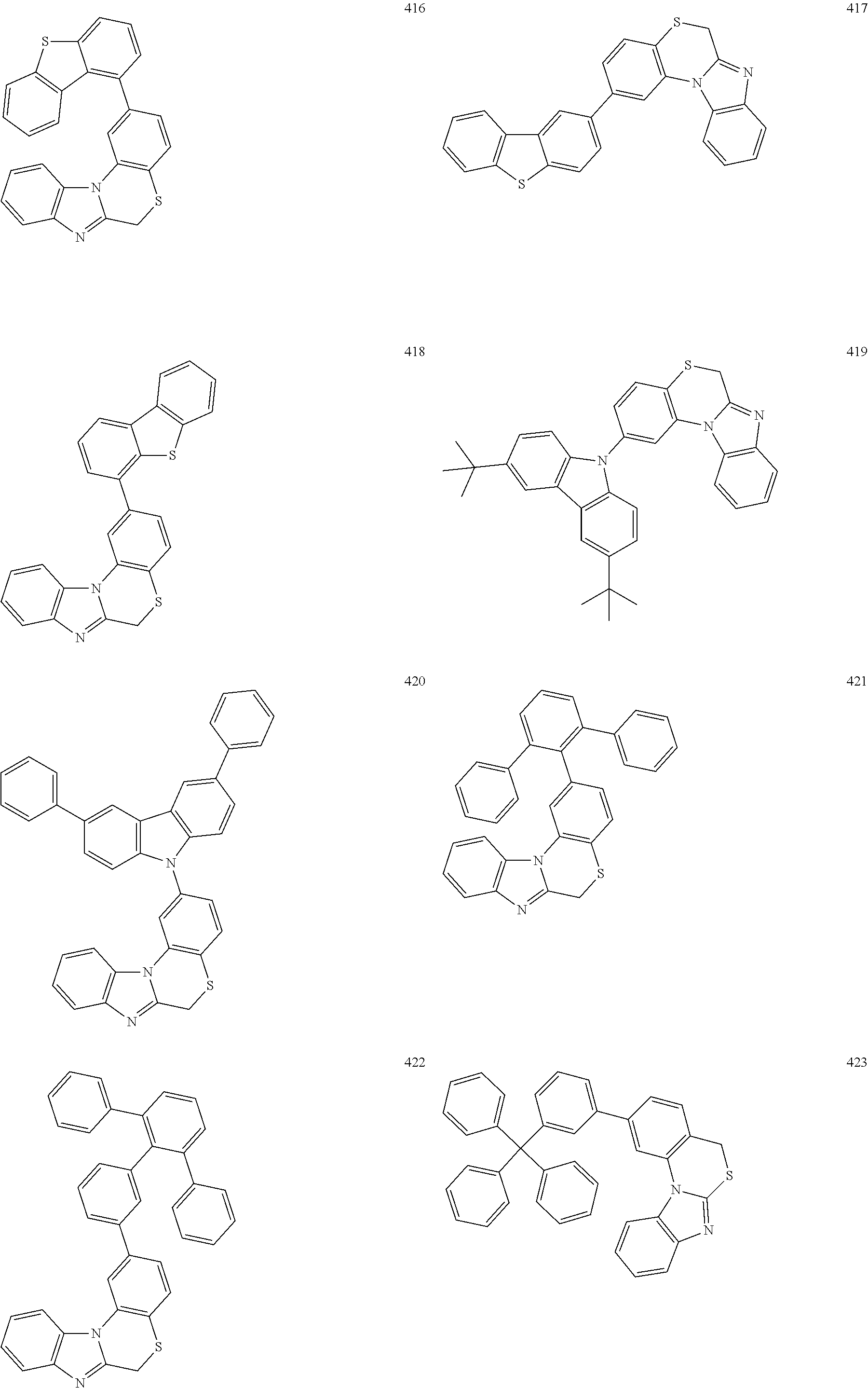

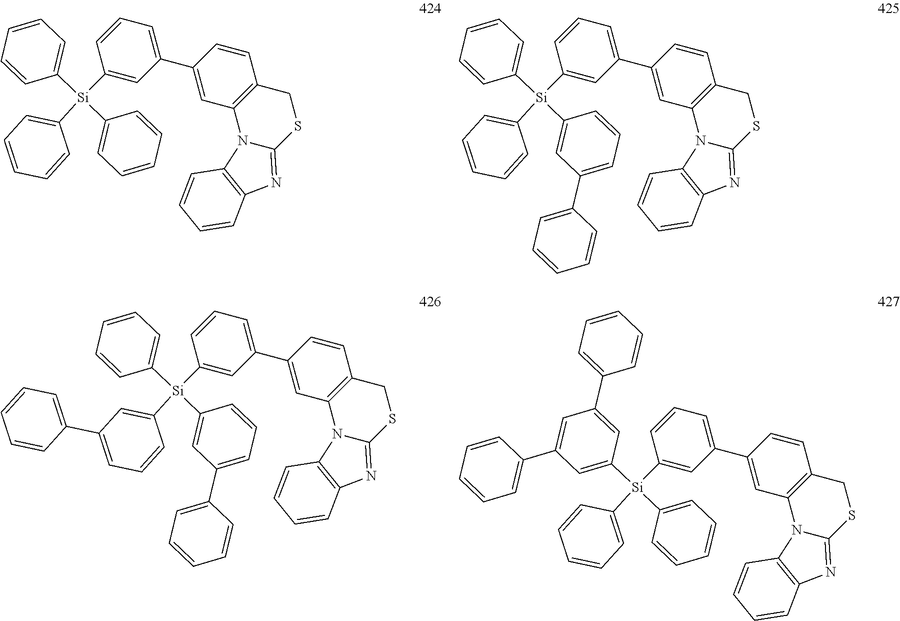 [SMILES: c1ccc([Si](c2ccccc2)(c2ccccc2)c2cccc(-c3ccc4c(c3)-n3c(nc5ccccc53)SC4)c2)cc1 ; c1ccc(-c2cccc([Si](c3ccccc3)(c3ccccc3)c3cccc(-c4ccc5c(c4)-n4c(nc6ccccc64)SC5)c3)c2)cc1 ; c1ccc(-c2cccc([Si](c3ccccc3)(c3cccc(-c4ccccc4)c3)c3cccc(-c4ccc5c(c4)-n4c(nc6ccccc64)SC5)c3)c2)cc1 ; c1ccc(-c2cc(-c3ccccc3)cc([Si](c3ccccc3)(c3ccccc3)c3cccc(-c4ccc5c(c4)-n4c(nc6ccccc64)SC5)c3)c2)cc1]

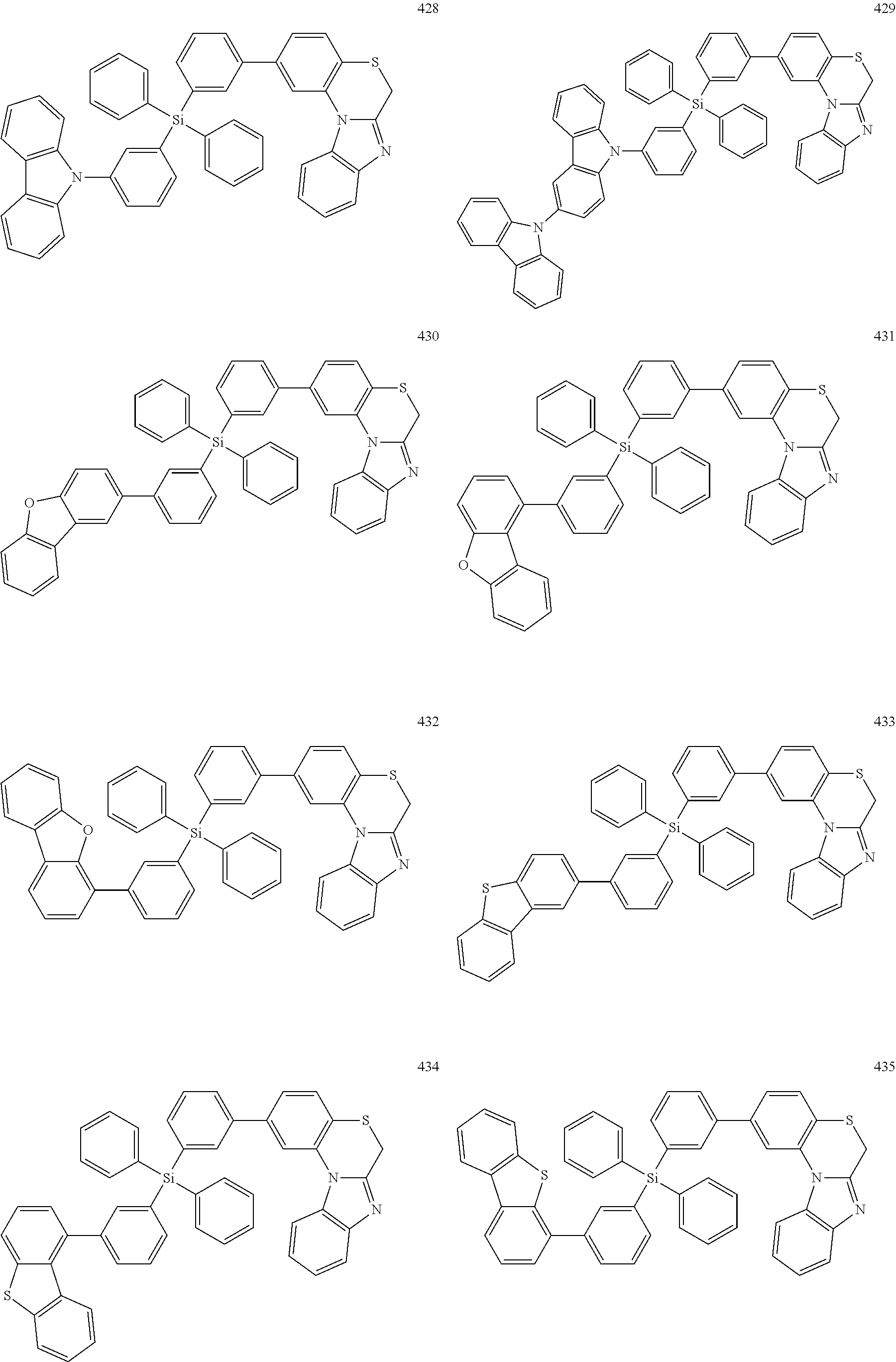

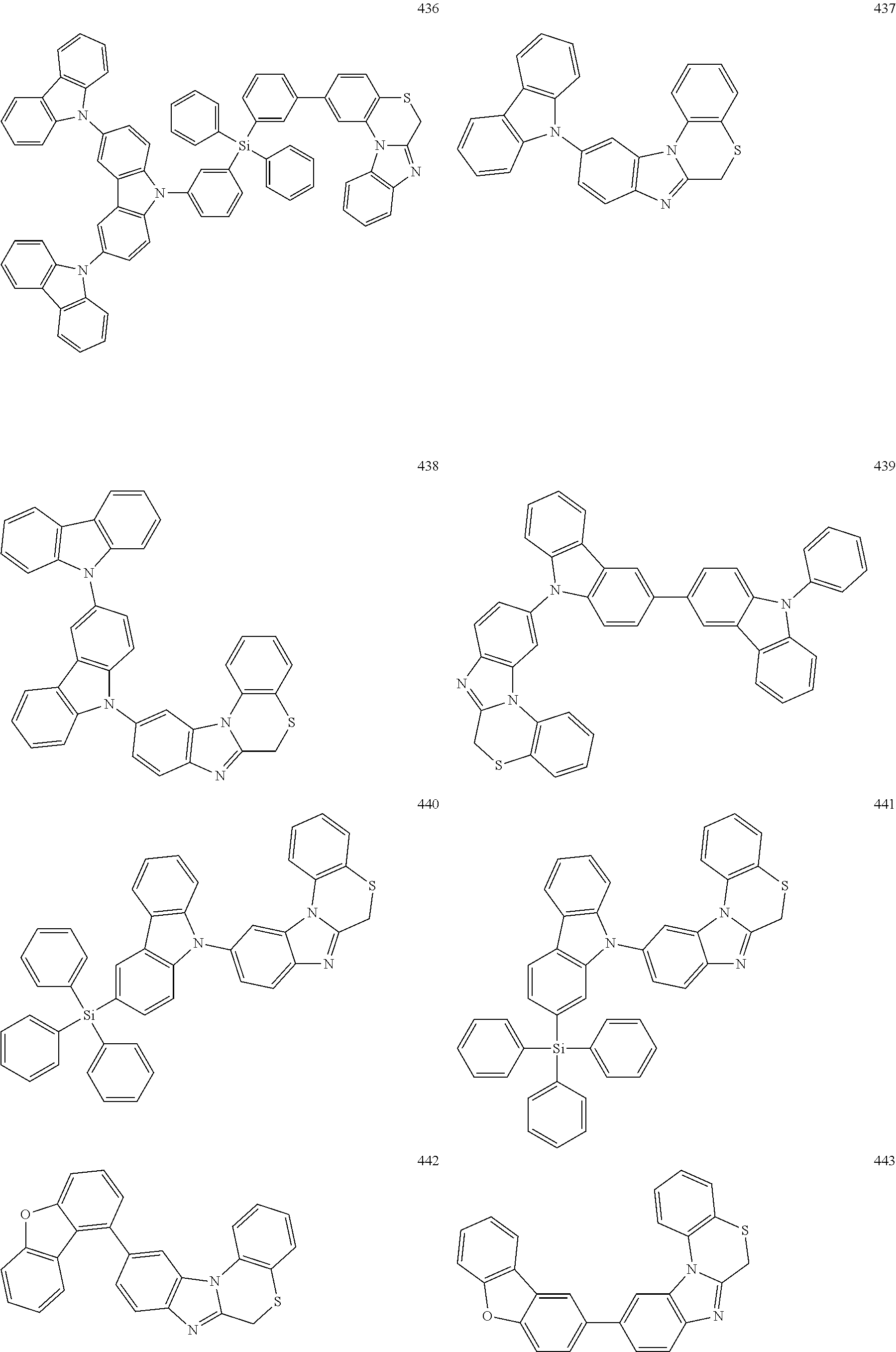

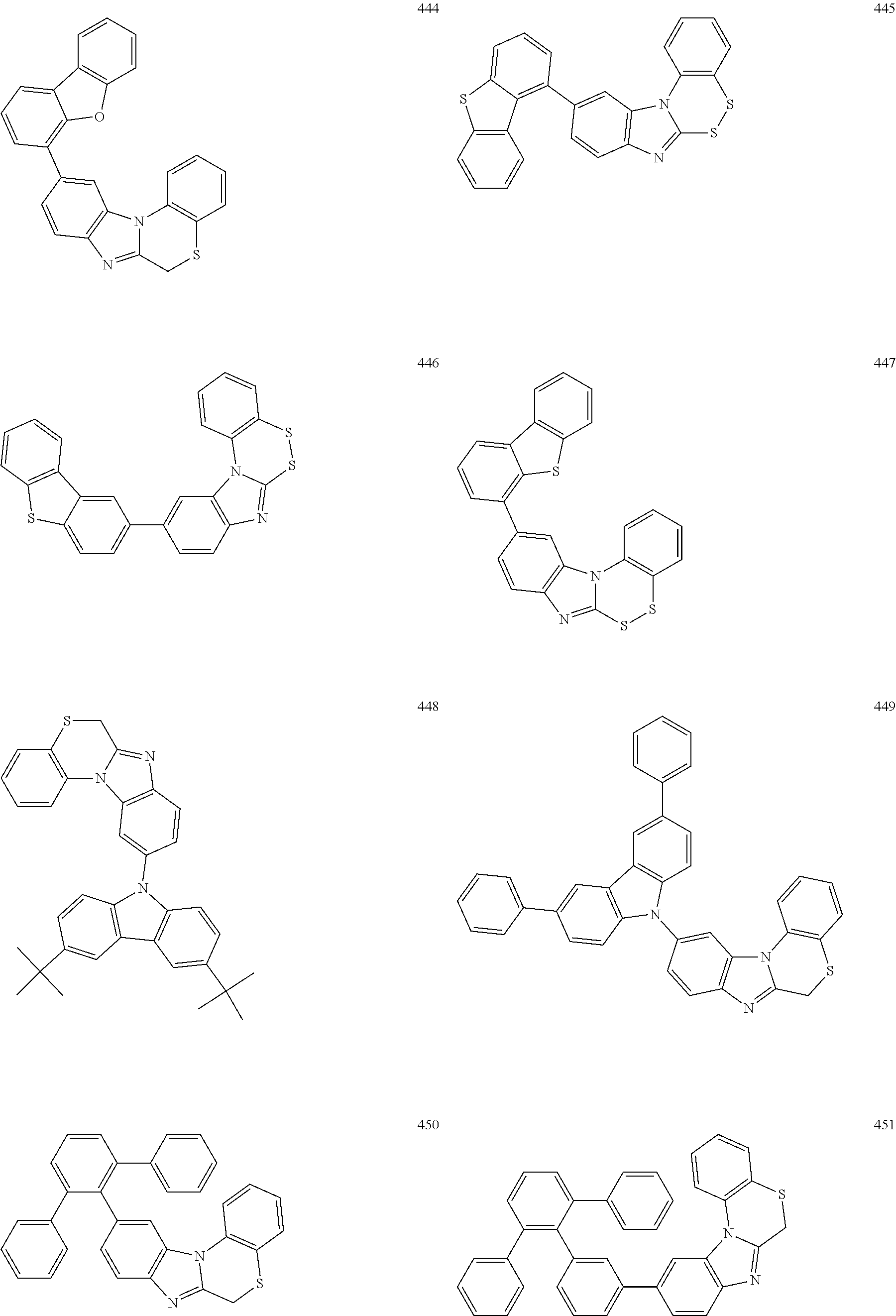

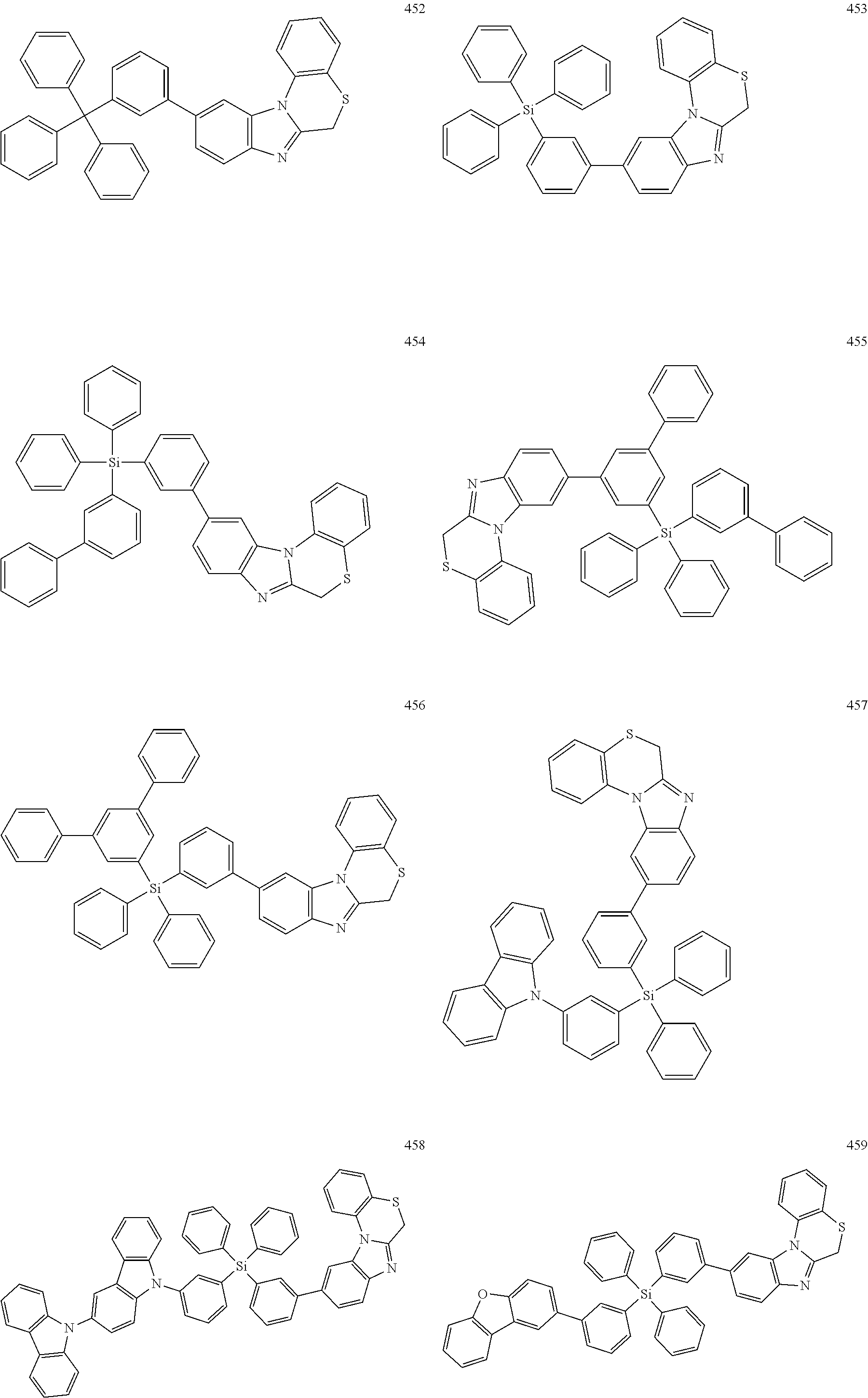

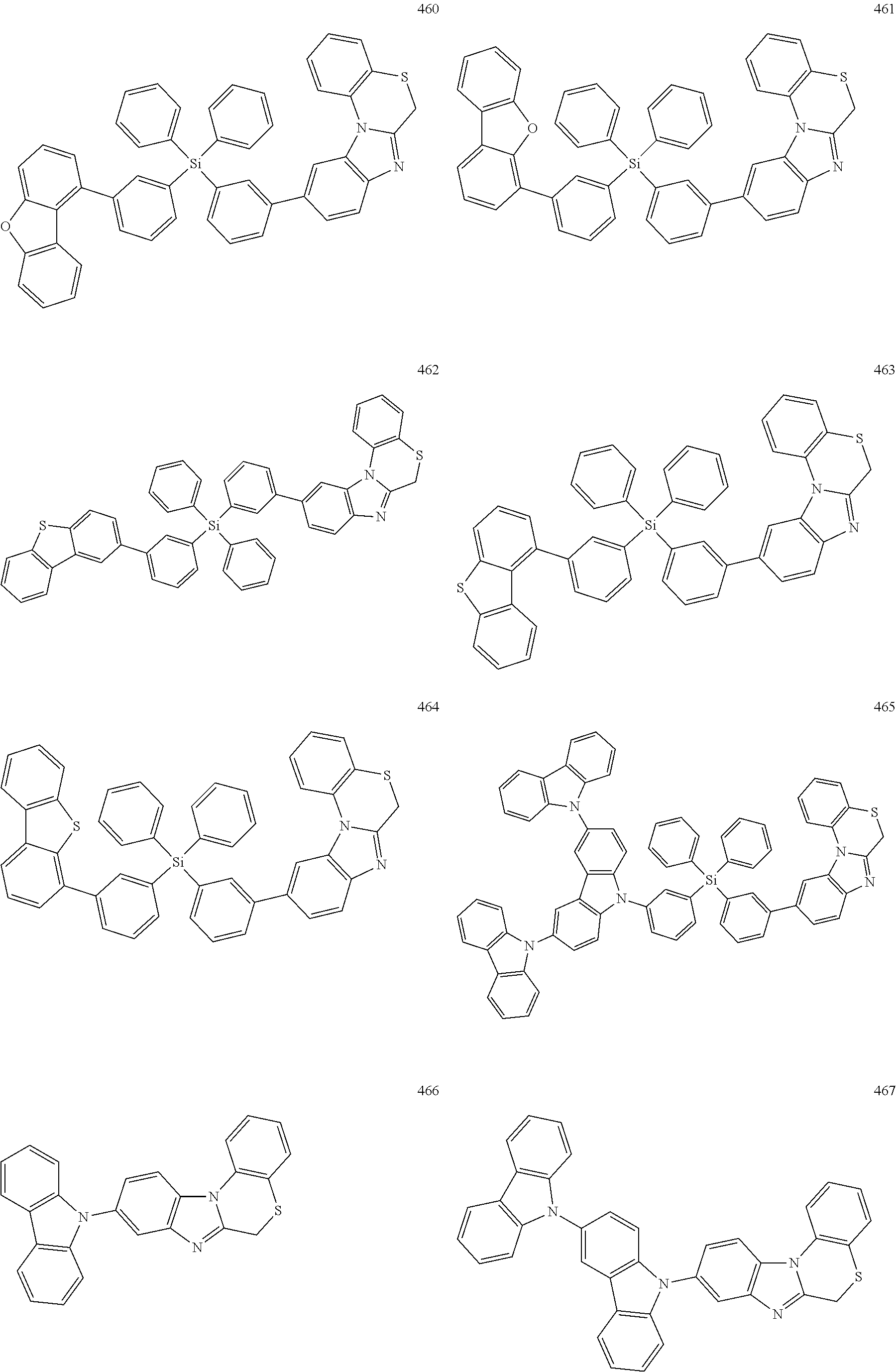

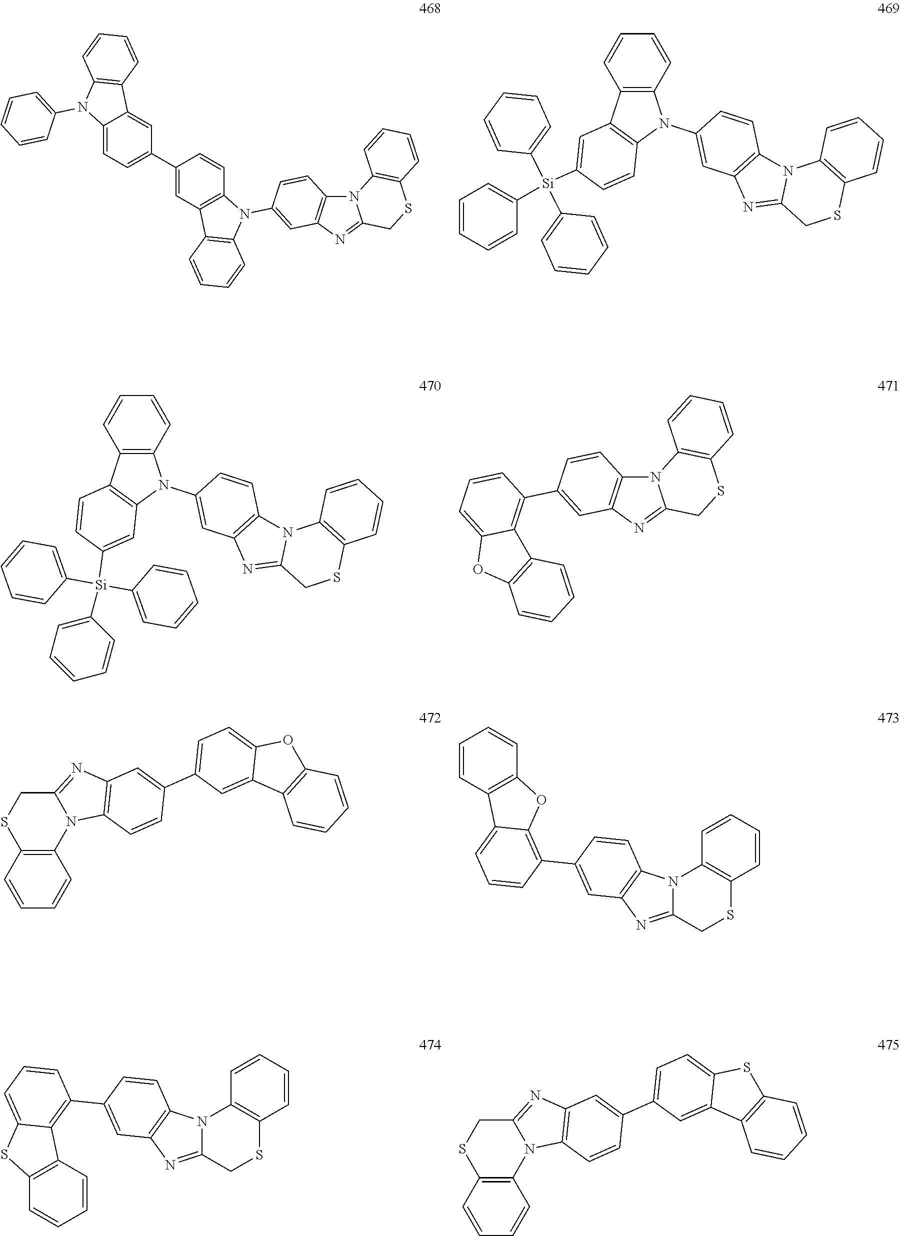

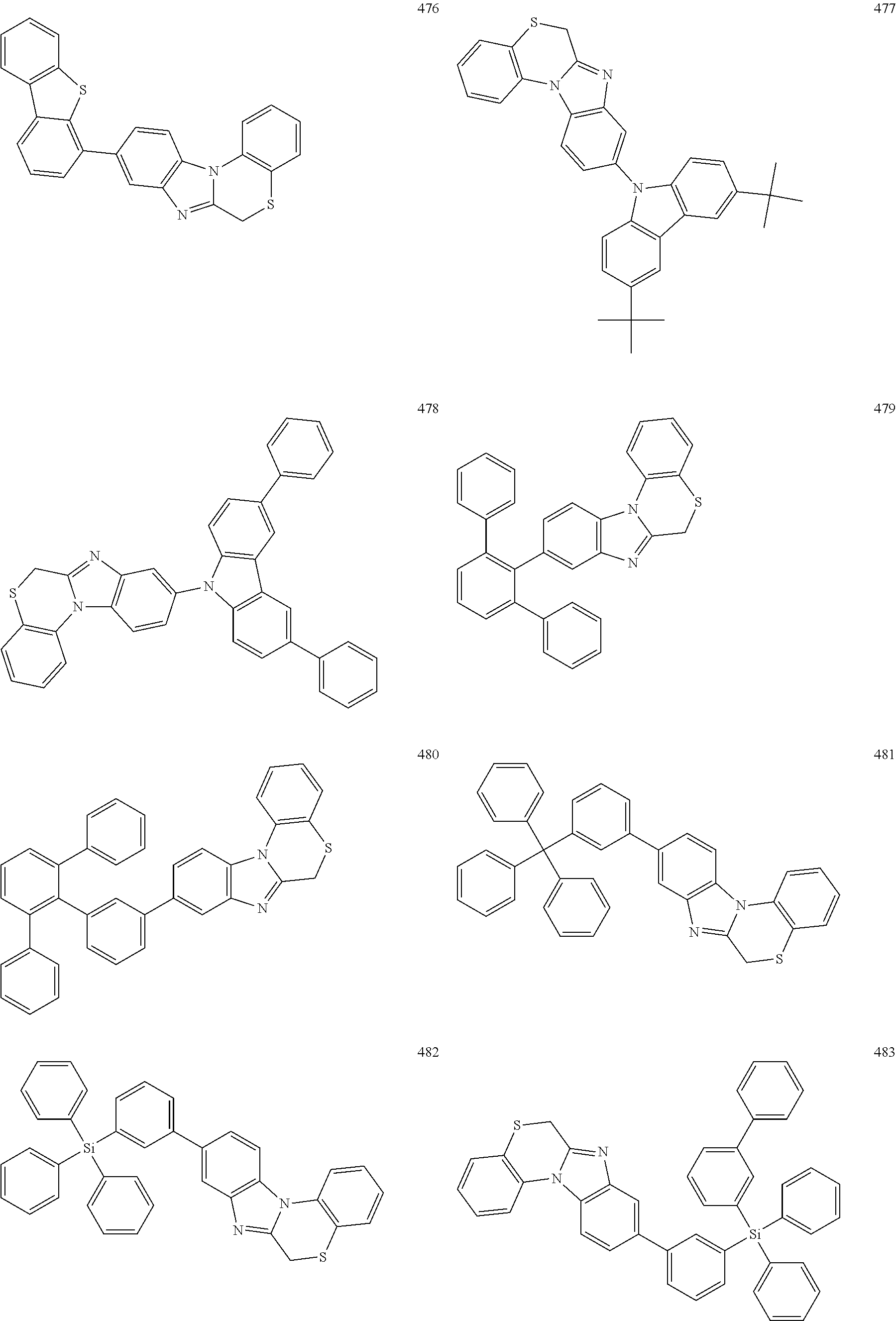

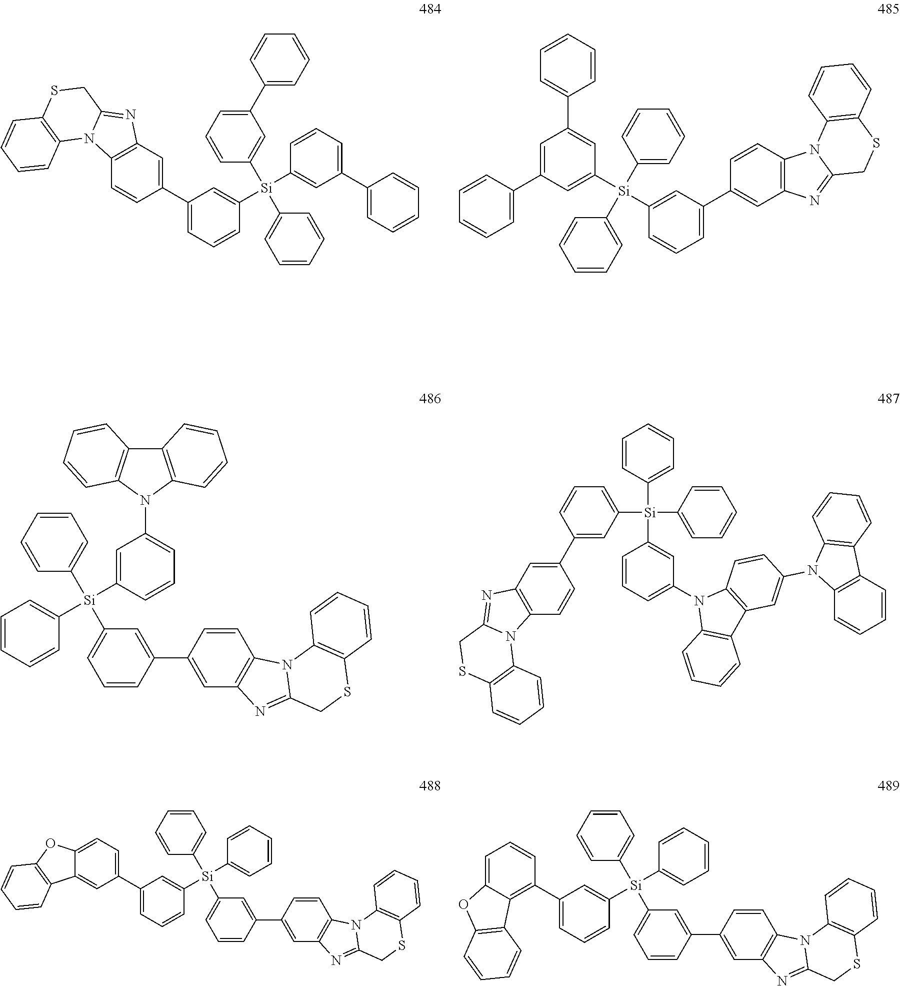

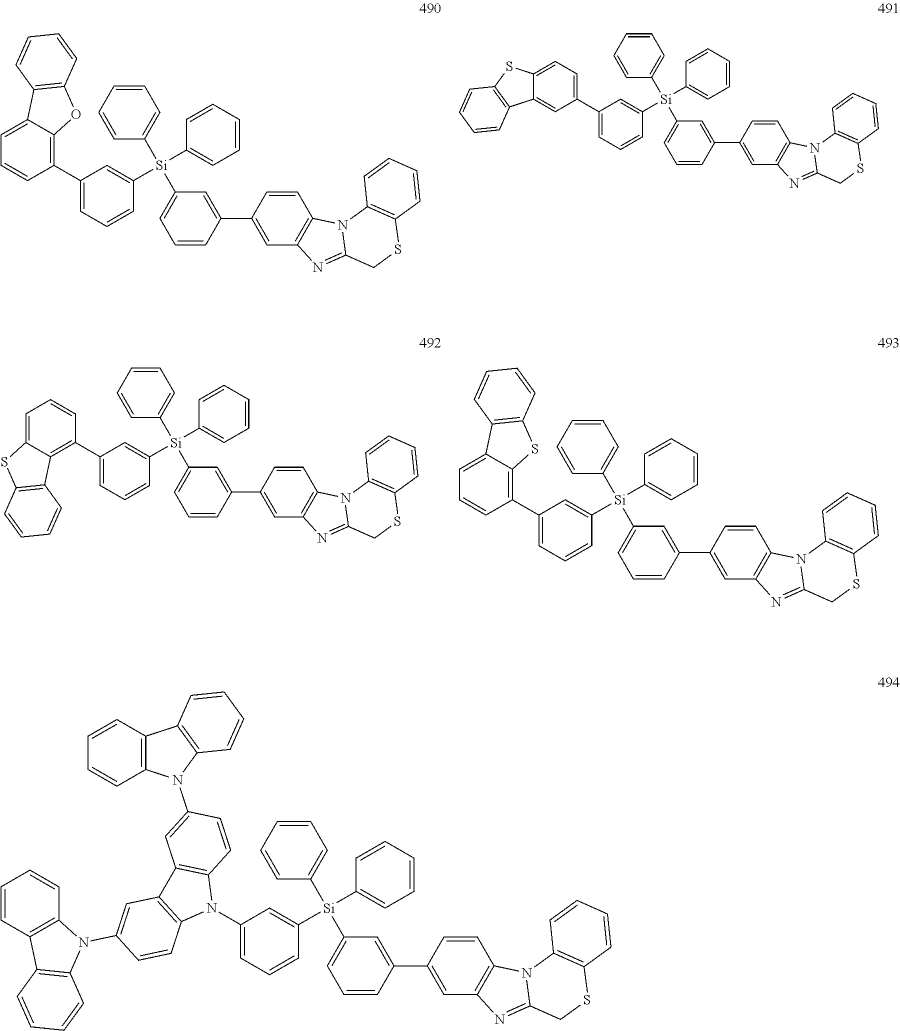

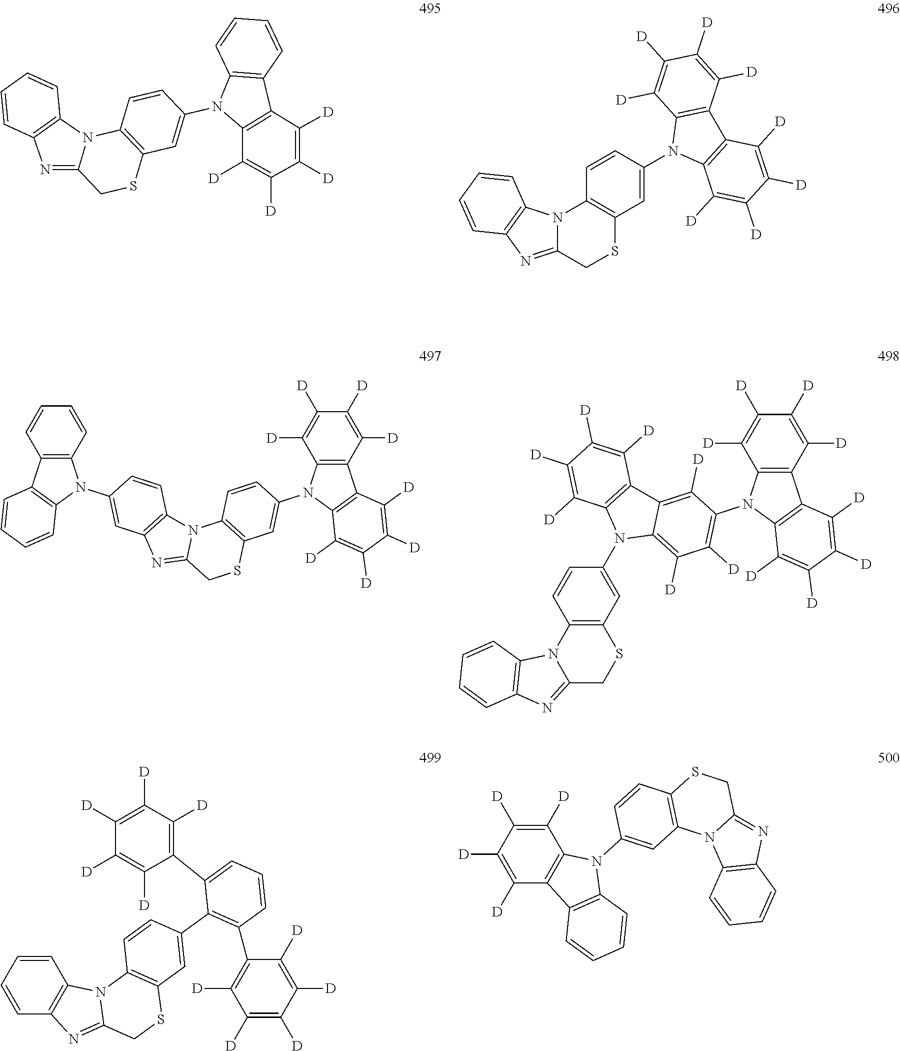

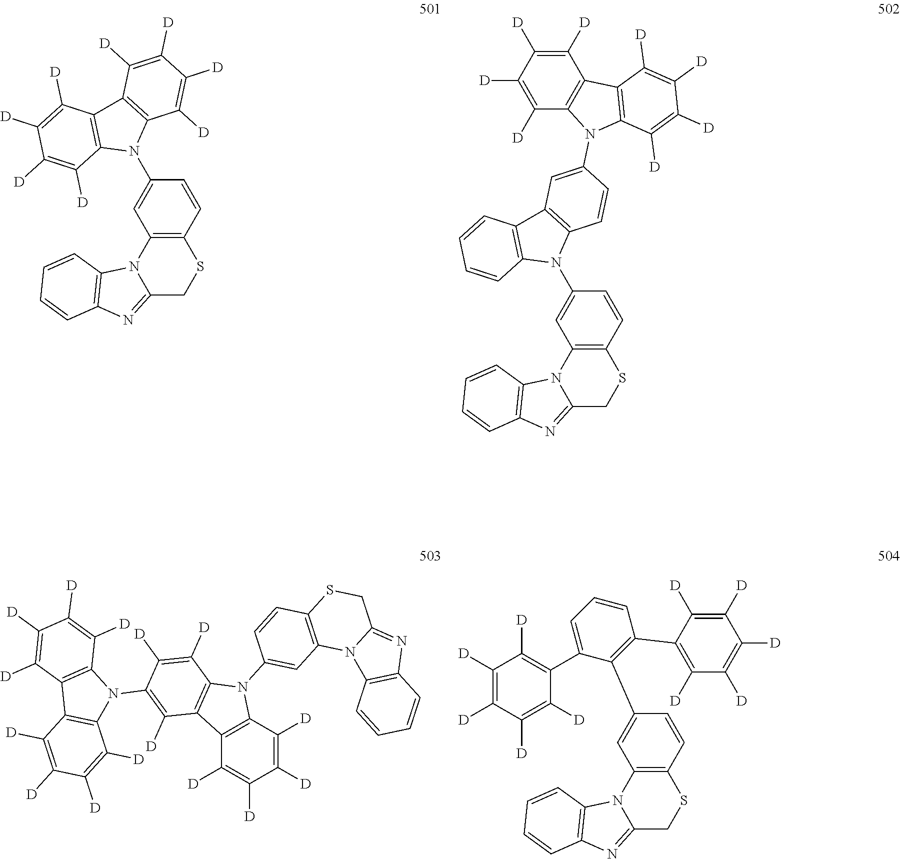 [SMILES: [2H]c1c([2H])c([2H])c2c(c1[2H])c1c([2H])c([2H])c([2H])c([2H])c1n2-c1ccc2c(c1)-n1c(nc3ccccc31)CS2 ; [2H]c1c([2H])c([2H])c2c(c1[2H])c1c([2H])c([2H])c([2H])c([2H])c1n2-c1ccc2c(c1)c1ccccc1n2-c1ccc2c(c1)-n1c(nc3ccccc31)CS2 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc2c(c1)-n1c(nc3ccccc31)CS2 ; [2H]c1c([2H])c([2H])c(-c2cccc(-c3c([2H])c([2H])c([2H])c([2H])c3[2H])c2-c2ccc3c(c2)-n2c(nc4ccccc42)CS3)c([2H])c1[2H]]

The compound represented by Formula 1 essentially includes O or S in a moiety represented by

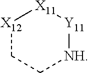

Accordingly, a) the planarity of the molecular structure may be lowered, and b) since the π-conjugation between the benzene rings outside the moiety is inhibited, the triplet energy level of the compound represented by Formula 1 may be increased.

In the heterocyclic compound represented by Formula 1, the molecular orbital of X₁₁ and/or X₁₂ may have a sp3 structure. Accordingly, since the moiety represented by

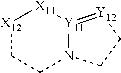

inhibits the pi-conjugation between the benzene rings outside the moiety, the lowest excitation triplet energy level of the heterocyclic compound may be increased. Thus, the heterocyclic compound may be suitable for use as a phosphorescent and/or a delayed fluorescence host.

In addition, due to a rigid structure in which several 5-membered rings and/or 6-membered rings are condensed, the heterocyclic compound has an increased glass transition temperature and/or thermal stability of the molecule.

In addition, since the heterocyclic compound contains a bulky substituent, the intermolecular attraction through large steric hindrance may be lowered and thus a relatively high lowest excitation triplet energy level may be obtained. Through this, when the heterocyclic compound is applied to a light-emitting device, the triplet exciton generated in an emission layer is inhibited from diffusing into a neighboring layer, for example, a hole transport layer or an electron transport layer. Accordingly, the luminescence efficiency may be improved. Therefore, the light-emitting device may exhibit high luminescence characteristics.

In addition, since the heterocyclic compound has a relatively high triplet energy level, the heterocyclic compound may be suitable for use as a blue host material.

Accordingly, an electronic device, for example, a light-emitting device, employing the heterocyclic compound represented by Formula 1, may have a low driving voltage, improved efficiency, and/or improved lifespan.

Synthesis methods of the heterocyclic compound represented by Formula 1 may be recognizable by one of ordinary skill in the art by referring to Synthesis Examples and/or Examples provided below.

At least one heterocyclic compound represented by Formula 1 may be used in a light-emitting device (for example, an organic light-emitting device). Accordingly, provided is a light-emitting device including: a first electrode; a second electrode facing the first electrode; and an interlayer located between the first electrode and the second electrode and including an emission layer, wherein the interlayer includes the heterocyclic compound represented by Formula 1.

In some embodiments,

the first electrode of the light-emitting device may be an anode,

the second electrode of the light-emitting device may be a cathode,

the interlayer further includes a hole transport region between the first electrode and the emission layer and an electron transport region between the emission layer and the second electrode,

the hole transport region includes a hole injection layer, a hole transport layer, an emission auxiliary layer, an electron blocking layer, or any combination thereof, and

the electron transport region may include a buffer layer, a hole blocking layer, an electron control layer, an electron transport layer, an electron injection layer, or any combination thereof.

In one or more embodiments, the heterocyclic compound may be included between the first electrode and the second electrode of the light-emitting device. Accordingly, the heterocyclic compound may be included in the interlayer of the light-emitting device, for example, in the emission layer of the interlayer.

In one or more embodiments, the emission layer of the interlayer of the light-emitting device may include a dopant and a host, and the heterocyclic compound may be included in the host. In other words, the heterocyclic compound may act as a host. The emission layer may emit red light, green light, blue light, and/or white light. For example, the emission layer may emit blue light. The blue light may have a maximum emission wavelength of, for example, about 400 nm to about 490 nm.

In one or more embodiments, the emission layer of the interlayer of the light-emitting device may include a dopant and a host, and the heterocyclic compound may be included in the host, and the dopant may emit blue light. For example, the dopant may include a phosphorescent dopant, a thermally activated delayed fluorescence dopant, or a combination thereof. The emission layer and the dopant may be the same as described in the present specification.

In one or more embodiments, the light-emitting device may include a capping layer located outside the first electrode and/or outside the second electrode. For example, the capping layer may include the heterocyclic compound represented by Formula 1.

In one or more embodiments, the light-emitting device may further include at least one of a first capping layer located outside the first electrode and a second capping layer located outside the second electrode, and the heterocyclic compound represented by Formula 1 may be included in at least one of the first capping layer, the second capping layer, or any combination thereof. The first capping layer and/or the second capping layer are the same as described in the present specification.

In one or more embodiments, the light-emitting device may further include:
a first capping layer located outside the first electrode and including the heterocyclic compound represented by Formula 1;
a second capping layer located outside the second electrode and including the heterocyclic compound represented by Formula 1; or
the first capping layer and the second capping layer.

The expression “(an interlayer and/or a capping layer) includes at least one heterocyclic compound represented by Formula 1” as used herein may include a case in which “(an interlayer and/or a capping layer) includes identical heterocyclic compounds represented by Formula 1” and a case in which “(an organic layer) includes two or more different heterocyclic compounds represented by Formula 1.”

For example, the interlayer and/or capping layer may include Compound 1 only as the heterocyclic compound. In this regard, Compound 1 may exist in the emission layer of the light-emitting device. In one or more embodiments, the interlayer may include, as the heterocyclic compound, Compound 1 and Compound 2. In this regard, Compound 1 and Compound 2 may exist in an identical layer (for example, Compound 1 and Compound 2 may all exist in an emission layer), or different layers (for example, Compound 1 may exist in an emission layer and Compound 2 may exist in an electron transport region).

The term “interlayer” as used herein refers to a single layer and/or all of a plurality of layers located between a first electrode and a second electrode of a light-emitting device.

Another aspect provides an electronic apparatus including the light-emitting device. The electronic apparatus may further include a thin-film transistor. In one or more embodiments, the electronic apparatus may further include a thin-film transistor including a source electrode and a drain electrode, and the first electrode of the light-emitting device may be electrically connected to the source electrode or the drain electrode. In one or more embodiments, the electronic apparatus may further include a color filter, a color conversion layer, a touch screen layer, a polarizing layer, or any combination thereof. More details on the electronic apparatus are the same as described in the present specification.

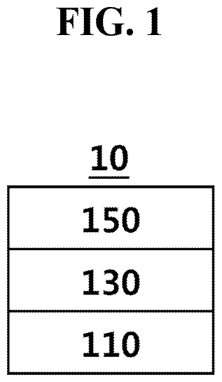

### Description of FIG. 1

FIG. 1 is a schematic cross-sectional view of a light-emitting device 10 according to an exemplary embodiment. The light-emitting device 10 includes a first electrode 110, an interlayer 130, and a second electrode 150.

Hereinafter, the structure of the light-emitting device 10 according to an embodiment and a method of manufacturing the light-emitting device 10 will be described in connection with FIG. 1.

### First Electrode 110

In FIG. 1, a substrate may be additionally located under the first electrode 110 or above the second electrode 150. As the substrate, a glass substrate or a plastic substrate may be used. In one or more embodiments, the substrate may be a flexible substrate, and may include plastics with excellent heat resistance and durability, such as polyimide, polyethylene terephthalate (PET), polycarbonate, polyethylene napthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high work function material that facilitates injection of holes.

The first electrode 110 may be a reflective electrode, a semi-transmissive electrode, or a transmissive electrode. For example, the first electrode may include indium tin oxide (ITO), indium zinc oxide (IZO), tin oxide (SnO₂), zinc oxide (ZnO), magnesium (Mg), silver (Ag), aluminum (Al), aluminum-lithium (Al—Li), calcium (Ca), magnesium-indium (Mg—In), magnesium-silver (Mg—Ag), or any combination thereof.

In one or more embodiments, when the first electrode 110 is a transmissive electrode, a material for forming the first electrode 110 may include indium tin oxide (ITO), indium zinc oxide (IZO), tin oxide (SnO₂), zinc oxide (ZnO), or any combinations thereof. In one or more embodiments, when the first electrode 110 is a semi-transmissive electrode or a reflective electrode, magnesium (Mg), silver (Ag), aluminum (Al), aluminum-lithium (Al—Li), calcium (Ca), magnesium-indium (Mg—In), magnesium-silver (Mg—Ag), or any combinations thereof may be used as a material for forming a first electrode.

The first electrode 110 may have a single layer consisting of a single-layered structure or a multilayer structure including a plurality of layers. For example, the first electrode 110 may have a three-layered structure of ITO/Ag/ITO.

### Interlayer 130

The interlayer 130 may be located on the first electrode 110. The interlayer 130 may include an emission layer.

The interlayer 130 may further include a hole transport region placed between the first electrode 110 and the emission layer and an electron transport region placed between the emission layer and the second electrode 150.

The interlayer 130 may further include metal-containing compounds such as organometallic compounds, inorganic materials such as quantum dots, and the like, in addition to various organic materials.

In one or more embodiments, the interlayer 130 may include, i) two or more emitting units sequentially stacked between the first electrode 110 and the second electrode 150 and ii) a charge generation layer located between the two emitting units. When the interlayer 130 includes the emitting unit and the charge generation layer as described above, the light-emitting device 10 may be a tandem light-emitting device.

### Hole Transport Region in Interlayer 130

The hole transport region may have: i) a single-layered structure consisting of a single layer consisting of a single material, ii) a single-layered structure consisting of a single layer consisting of a plurality of different materials, or iii) a multi-layered structure including a plurality of layers including different materials.

The hole transport region may include a hole injection layer, a hole transport layer, an emission auxiliary layer, an electron blocking layer, or any combination thereof.

For example, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, wherein, in each structure, layers are stacked sequentially from the first electrode 110.

The hole transport region may include a compound represented by Formula 201, a compound represented by Formula 202, or any combination thereof:

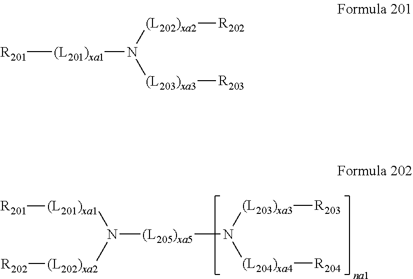

wherein, in Formulae 201 and 202,

L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

L₂₀₅ may be *—O—*′, *—S—*′, *—N(Q₂₀₁)—*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,

xa1 to xa4 may each independently an integer selected from 0 to 5,

xa5 may be an integer selected from 1 to 10,

R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

R₂₀₁ and R₂₀₂ may optionally be linked to each other, via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (for example, a carbazole group or the like) unsubstituted or substituted with at least one R₁₀ₐ (for example, Compound HT16),

R₂₀₃ and R₂₀₄ may optionally be linked to each other, via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and

na1 is an integer selected from 1 to 4.

In one or more embodiments, each of Formulae 201 and 202 may include at least one group represented by Formulae CY201 to CY217.

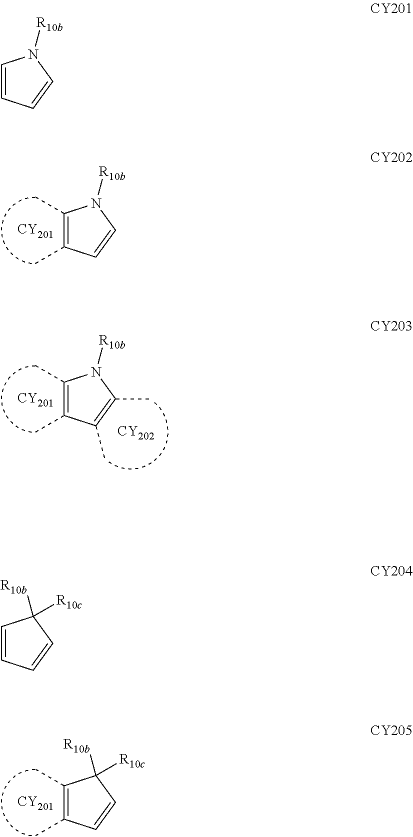

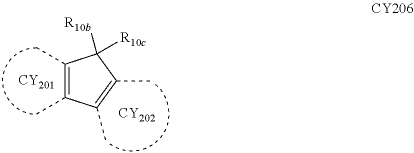

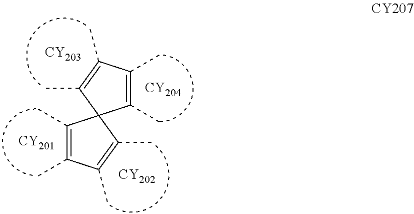

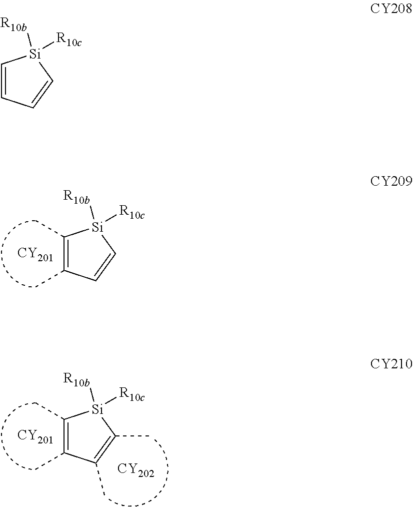

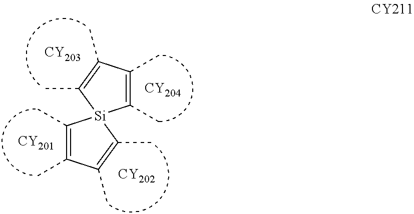

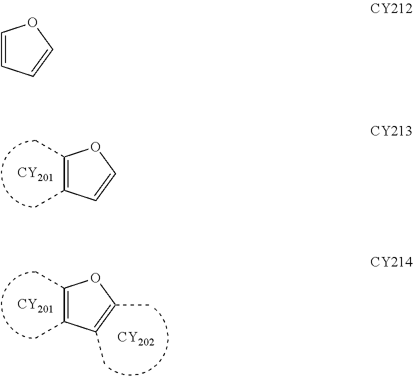

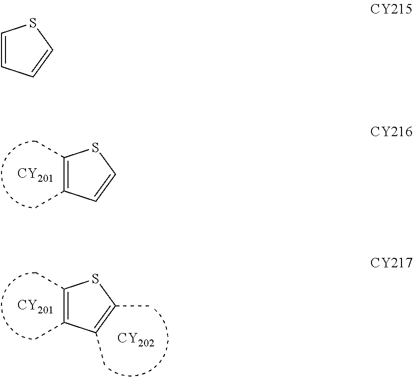

R_{10b} and R_{10c} in Formulae CY201 to CY217 are the same as described in connection with R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In one or more embodiments, ring CY₂₀₁ to ring CY₂₀₄ in Formulae CY201 to CY217 may each independently be a benzene group, a naphthalene group, a phenanthrene group, or an anthracene group.

In one or more embodiments, each of Formulae 201 and 202 may include at least one group represented by Formulae CY201 to CY203.

In one or more embodiments, Formula 201 may include at least one group represented by Formulae CY201 to CY203 and at least one group represented by Formulae CY204 to CY217.

In one or more embodiments, xa1 in Formula 201 is 1, R₂₀₁ is a group represented by one of Formulae CY201 to CY203, xa2 may be 0, and R₂₀₂ may be a group represented by one of Formulae CY204 to CY207.

In one or more embodiments, each of Formulae 201 and 202 may not include a group represented by one of Formulae CY201 to CY203.

In one or more embodiments, each of Formulae 201 and 202 may not include a group represented by one of Formulae CY201 to CY203, and may include at least one group represented by Formulae CY204 to CY217.

In one or more embodiments, each of Formulae 201 and 202 may not include a group represented by one of Formulae CY201 to CY217.

In one or more embodiments, the hole transport region may include one of Compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

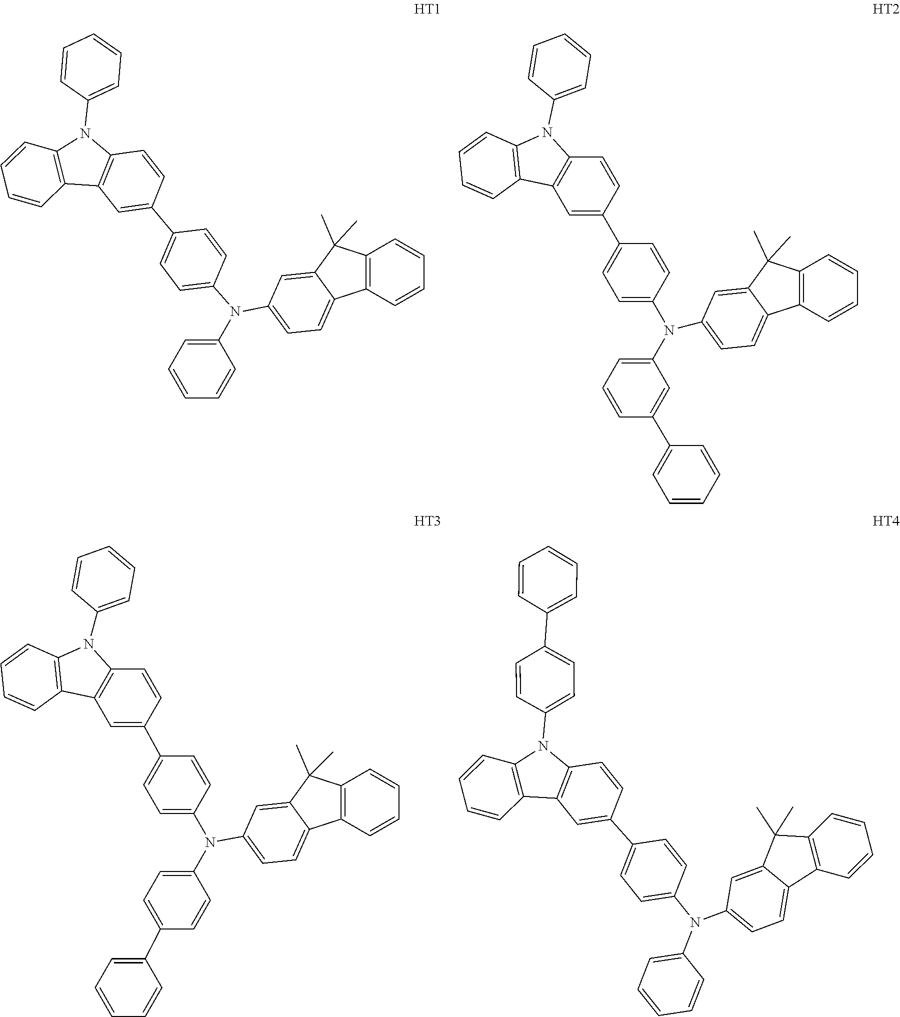 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

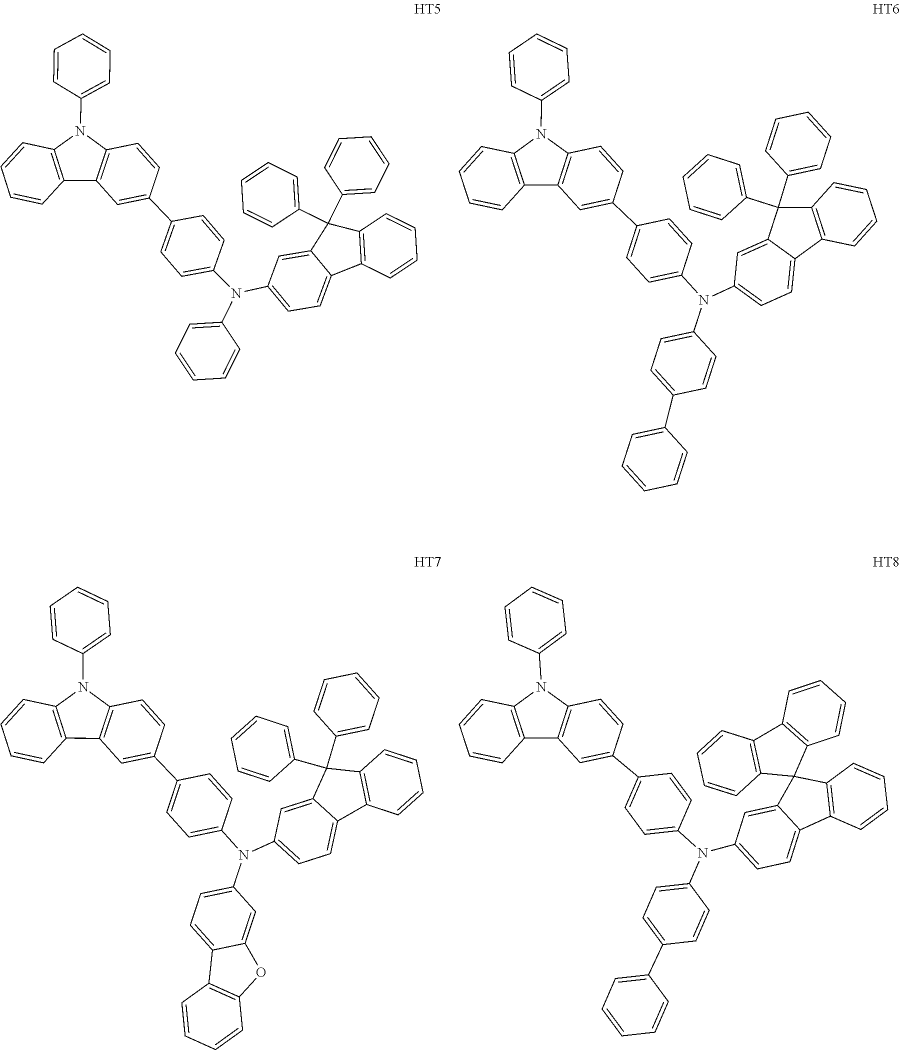 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

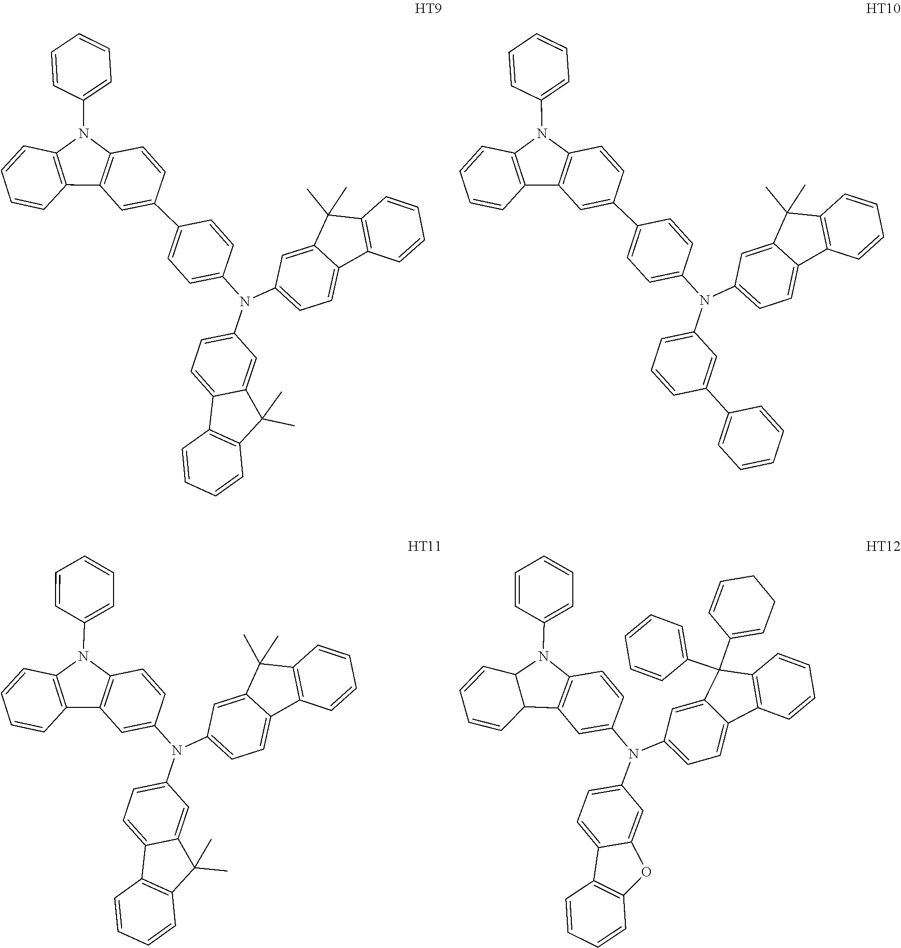 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; C1=CC2c3cc(N(c4ccc5c(c4)C(C4=CCCC=C4)(c4ccccc4)c4ccccc4-5)c4ccc5c(c4)oc4ccccc45)ccc3N(c3ccccc3)C2C=C1]

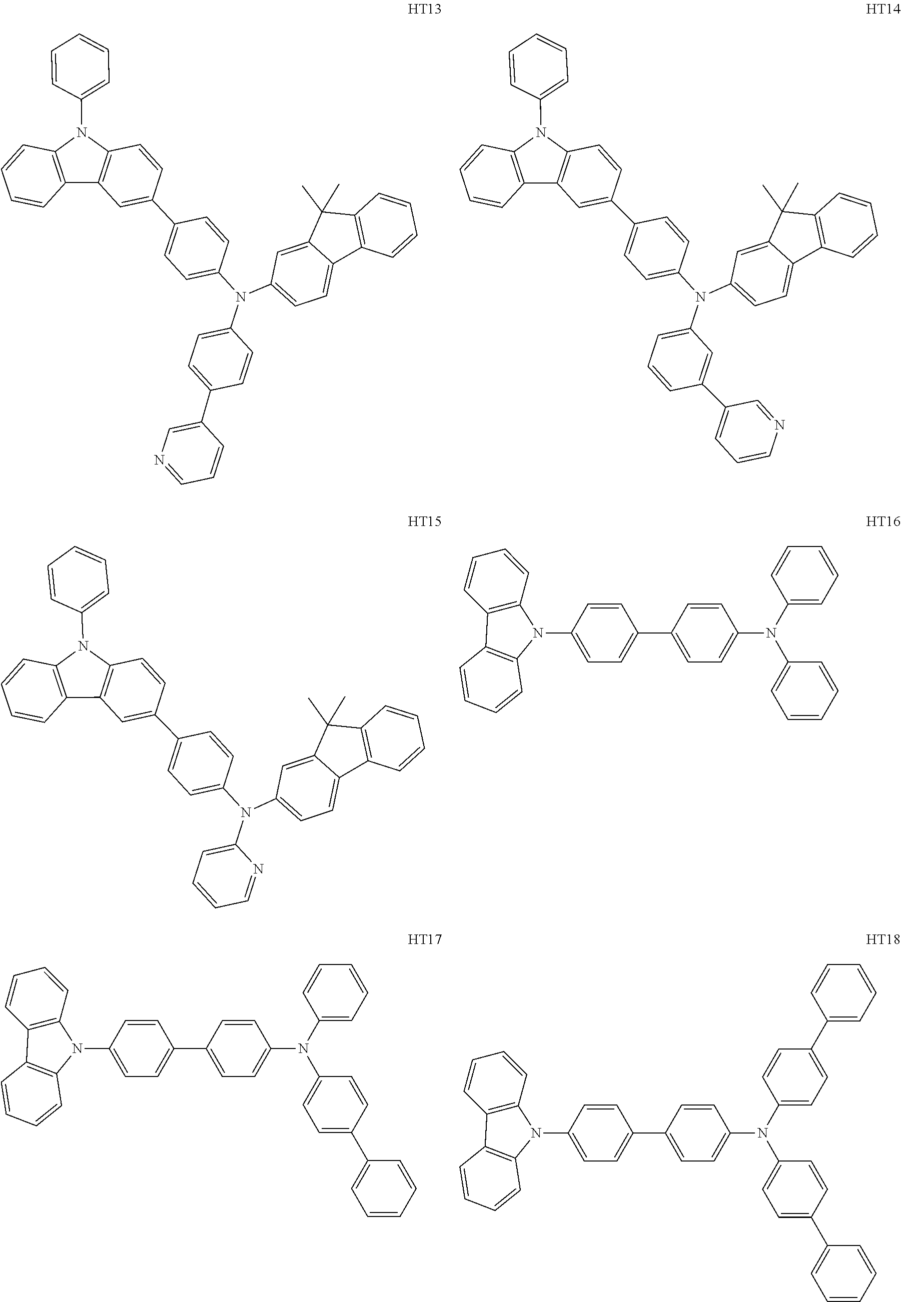 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1 ; c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

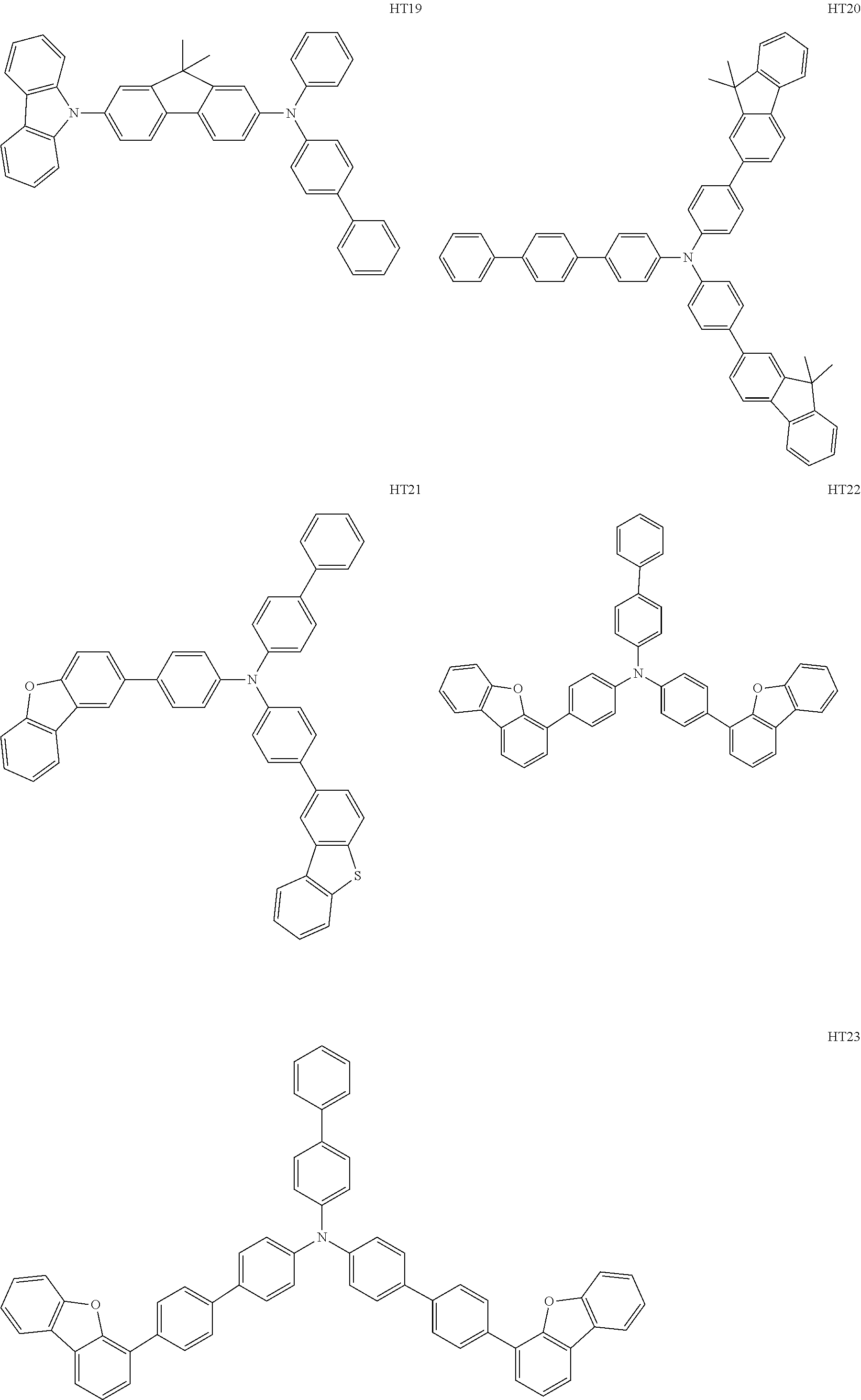

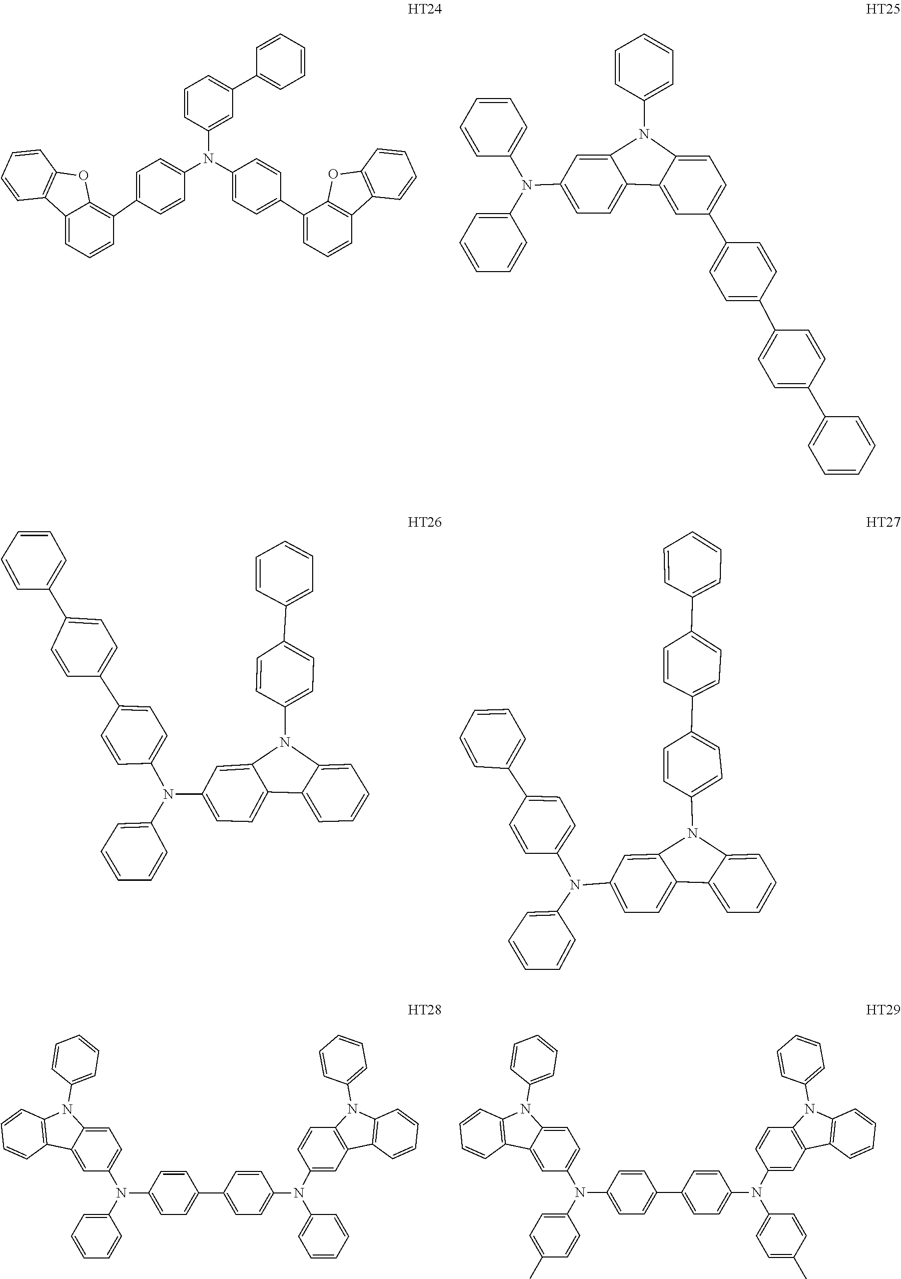

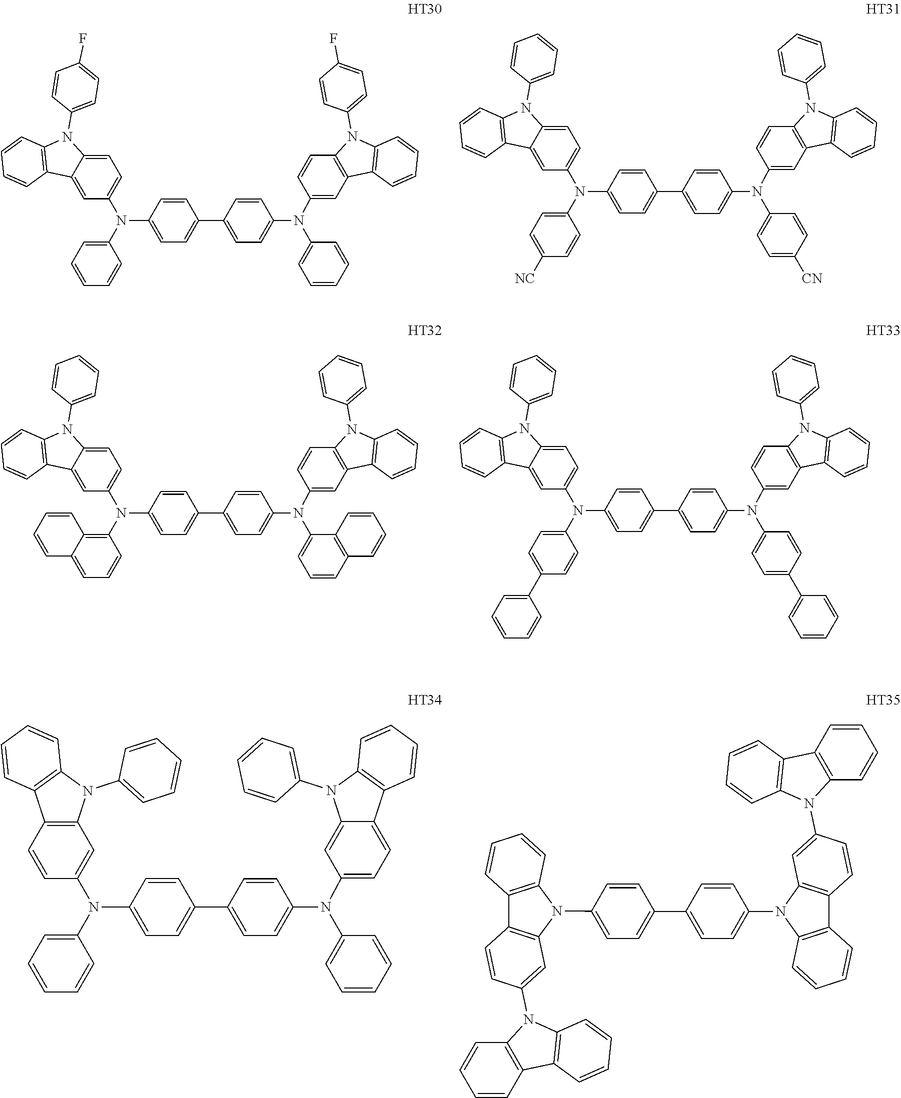

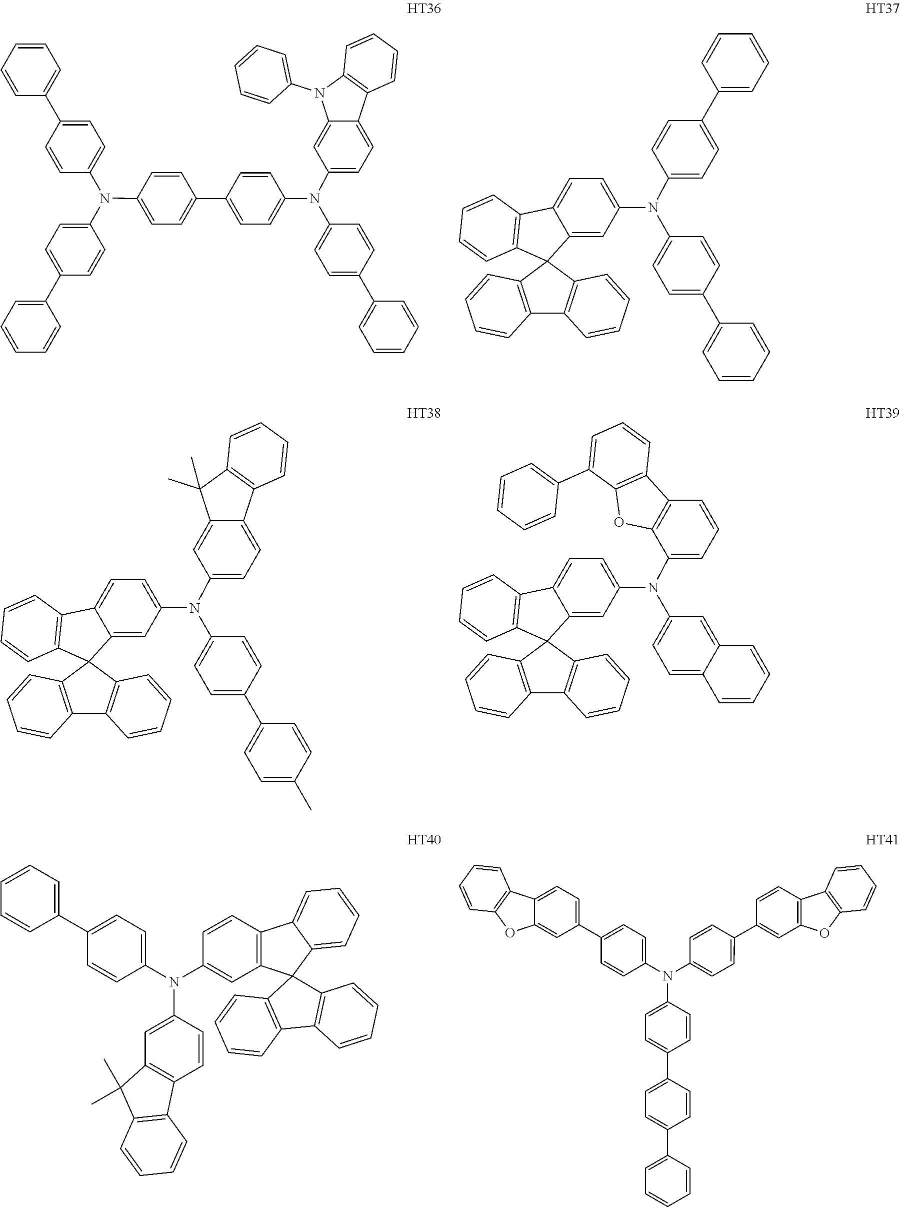

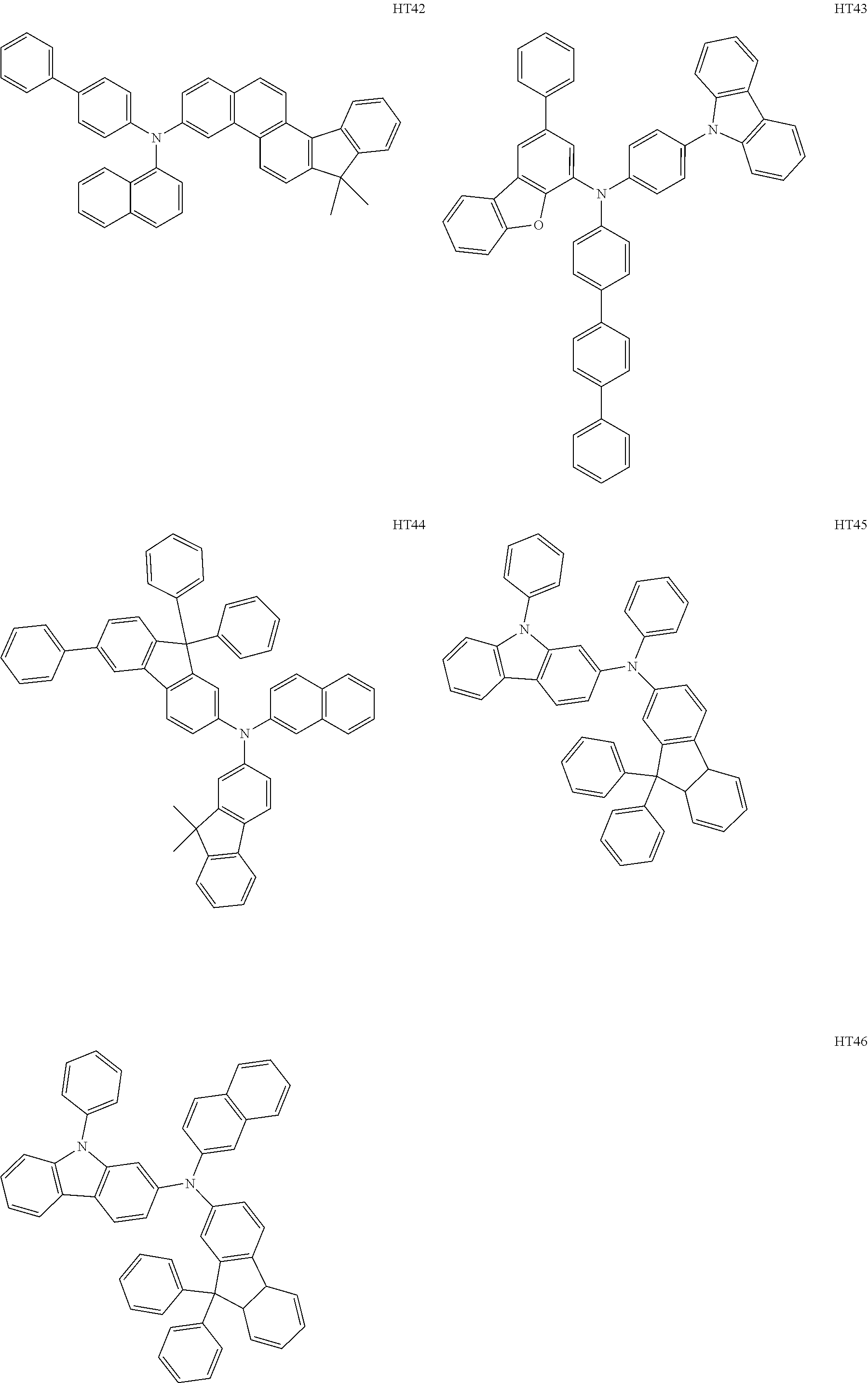 [SMILES: CC1(C)c2ccccc2-c2c1ccc1c2ccc2ccc(N(c3ccc(-c4ccccc4)cc3)c3cccc4ccccc34)cc21 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; CC1(C)c2ccccc2-c2ccc(N(c3ccc4c(c3)C(c3ccccc3)(c3ccccc3)c3ccc(-c5ccccc5)cc3-4)c3ccc4ccccc4c3)cc21 ; C1=CC2c3ccc(N(c4ccccc4)c4ccc5c6ccccc6n(-c6ccccc6)c5c4)cc3C(c3ccccc3)(c3ccccc3)C2C=C1 ; C1=CC2c3ccc(N(c4ccc5ccccc5c4)c4ccc5c6ccccc6n(-c6ccccc6)c5c4)cc3C(c3ccccc3)(c3ccccc3)C2C=C1]

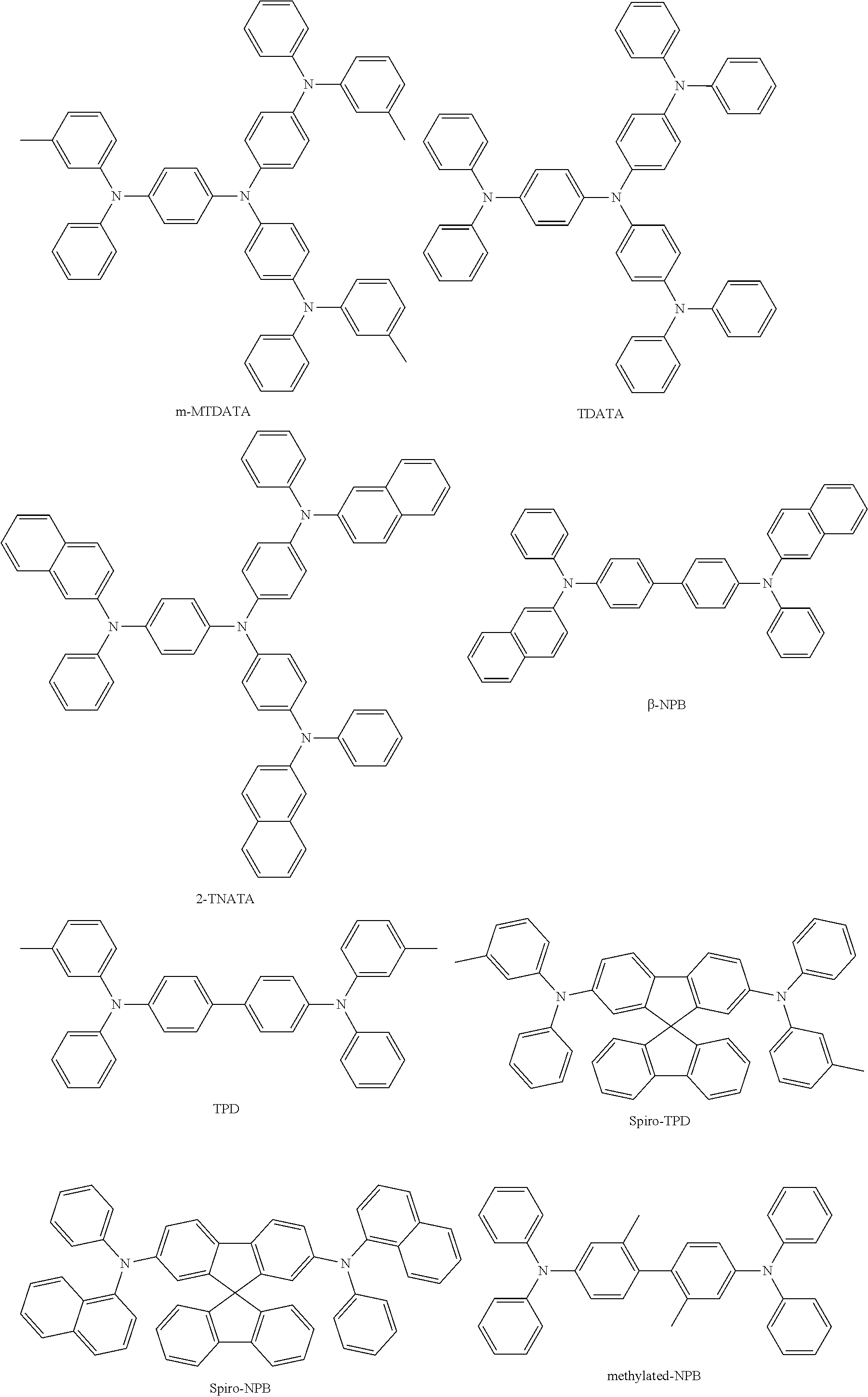

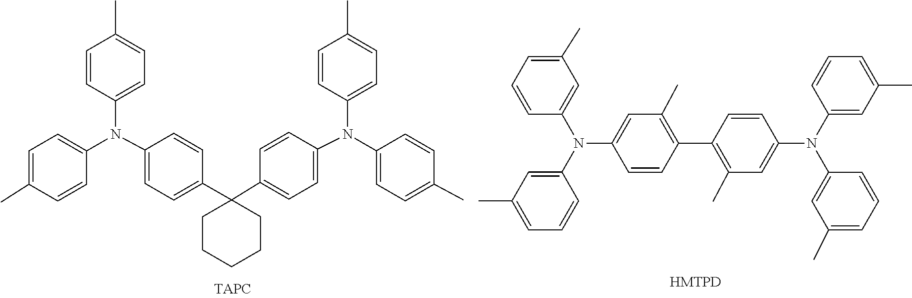

A thickness of the hole transport region may be in a range of about 50 Å to about 10,000 Å, for example, about 100 Å to about 4,000 Å. When the hole transport region includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer and the hole transport layer are within these ranges, satisfactory hole transporting characteristics may be obtained without a substantial increase in driving voltage.

The emission auxiliary layer may increase light-emission efficiency by compensating for an optical resonance distance according to the wavelength of light emitted by an emission layer, and the electron blocking layer may block the leakage of electrons from an emission layer to a hole transport region. Materials that may be included in the hole transport region may be included in the emission auxiliary layer and the electron blocking layer.

p-Dopant

The hole transport region may further include, in addition to these materials, a charge-generation material for the improvement of conductive properties. The charge-generation material may be uniformly or non-uniformly dispersed in the hole transport region (for example, in the form of a single layer consisting of a charge-generation material).

The charge-generation material may be, for example, a p-dopant.

In one embodiment, the lowest unoccupied molecular orbital (LUMO) energy level of the p-dopant may be −3.5 eV or less.

In one or more embodiments, the p-dopant may include a quinone derivative, a cyano group-containing compound, a compound containing element EL1 and element EL2, or any combination thereof.

Examples of the quinone derivative are TCNQ, F4-TCNQ, and the like.

Examples of the cyano group-containing compound are HAT-CN, and a compound represented by Formula 221 below.

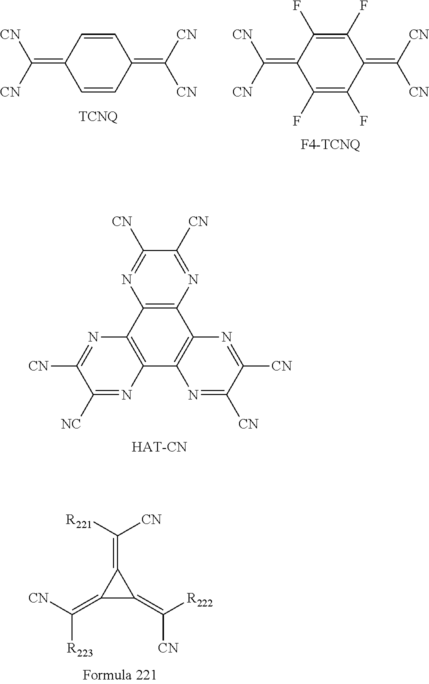

In Formula 221,

R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and

at least one of R₂₂₁ to R₂₂₃ may each independently be a C₃-C₆₀ carbocyclic group or a C₁-C₆₀ heterocyclic group, each substituted with a cyano group; —F; —Cl; —Br; —I; a C₁-C₂₀ alkyl group substituted with a cyano group, —F, —Cl, —Br, —I, or any combination thereof; or any combination thereof.

In the compound containing element EL1 and element EL2, element EL1 may be metal, metalloid, or a combination thereof, and element EL2 may be non-metal, metalloid, or a combination thereof.

Examples of the metal are an alkali metal (for example, lithium (Li), sodium (Na), potassium (K), rubidium (Rb), cesium (Cs), etc.); alkaline earth metal (for example, beryllium (Be), magnesium (Mg), calcium (Ca), strontium (Sr), barium (Ba), etc.); transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); post-transition metal (for example, zinc (Zn), indium (In), tin (Sn), etc.); and lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.).

Examples of the metalloid are silicon (Si), antimony (Sb), and tellurium (Te).

Examples of the non-metal are oxygen (O) and halogen (for example, F, Cl, Br, I, etc.).

In one or more embodiments, examples of the compound containing element EL1 and element EL2 are metal oxide, metal halide (for example, metal fluoride, metal chloride, metal bromide, or metal iodide), metalloid halide (for example, metalloid fluoride, metalloid chloride, metalloid bromide, or metalloid iodide), metal telluride, or any combination thereof.

Examples of the metal oxide include tungsten oxide (for example, WO, W₂O₃, WO₂, WO₃, W₂O₅, etc.), vanadium oxide (for example, VO, V₂O₃, VO₂, V₂O₅, etc.), molybdenum oxide (MoO, Mo₂O₃, MoO₂, MoO₃, Mo₂O₅, etc.), and rhenium oxide (for example, ReO₃, etc.).

Examples of the metal halide include alkali metal halide, alkaline earth metal halide, transition metal halide, post-transition metal halide, and lanthanide metal halide.

Examples of the alkali metal halide are LiF, NaF, KF, RbF, CsF, LiCl, NaCl, KCl, RbCl, CsCl, LiBr, NaBr, KBr, RbBr, CsBr, LiI, NaI, KI, RbI, and CsI.

Examples of the alkaline earth metal halide include BeF₂, MgF₂, CaF₂, SrF₂, BaF₂, BeCl₂, MgCl₂, CaCl₂, SrCl₂, BaCl₂, BeBr₂, MgBr₂, CaBr₂, SrBr₂, BaBr₂, BeI₂, MgI₂, CaI₂, SrI₂, and BaI₂.

Examples of the transition metal halide include titanium halide (for example, TiF₄, TiCl₄, TiBr₄, TiI₄, etc.), zirconium halide (for example, ZrF₄, ZrCl₄, ZrBr₄, ZrI₄, etc.), hafnium halide (for example, HfF₄, HfCl₄, HfBr₄, HfI₄, etc.), vanadium halide (for example, VF₃, VCl₃, VBr₃, VI₃, etc.), niobium halide (for example, NbF₃, NbCl₃, NbBr₃, NbI₃, etc.), tantalum halide (for example, TaF₃, TaCl₃, TaBr₃, TaI₃, etc.), chromium halide (for example, CrF₃, CrCl₃, CrBr₃, CrI₃, etc.), molybdenum halide (for example, MoF₃, MoCl₃, MoBr₃, MoI₃, etc.), tungsten halide (for example, WF₃, WCl₃, WBr₃, WI₃, etc.), manganese halide (for example, MnF₂, MnCl₂, MnBr₂, MnI₂, etc.), technetium halide (for example, TcF₂, TcCl₂, TcBr₂, TcI₂, etc.), rhenium halide (for example, ReF₂, ReCl₂, ReBr₂, ReI₂, etc.), iron halide (for example, FeF₂, FeCl₂, FeBr₂, FeI₂, etc.), ruthenium halide (for example, RuF₂, RuCl₂, RuBr₂, RuI₂, etc.), osmium halide (for example, OsF₂, OsCl₂, OsBr₂, OsI₂, etc.), cobalt halide (for example, CoF₂, CoCl₂, CoBr₂, CoI₂, etc.), rhodium halide (for example, RhF₂, RhCl₂, RhBr₂, RhI₂, etc.), iridium halide (for example, IrF₂, IrCl₂, IrBr₂, IrI₂, etc.), nickel halide (for example, NiF₂, NiCl₂, NiBr₂, NiI₂, etc.), palladium halide (for example, PdF₂, PdCl₂, PdBr₂, PdI₂, etc.), platinum halide (for example, PtF₂, PtCl₂, PtBr₂, PtI₂, etc.), copper halide (for example, CuF, CuCl, CuBr, CuI, etc.), silver halide (for example, AgF, AgCl, AgBr, AgI, etc.), and gold halide (for example, AuF, AuCl, AuBr, AuI, etc.).

Examples of the post-transition metal halide include zinc halide (for example, ZnF₂, ZnCl₂, ZnBr₂, ZnI₂, etc.), indium halide (for example, InI₃, etc.), and tin halide (for example, SnI₂, etc.).

Examples of the lanthanide metal halide include YbF, YbF₂, YbF₃, SmF₃, YbCl, YbCl₂, YbCl₃ SmCl₃, YbBr, YbBr₂, YbBr₃ SmBr₃, YbI, YbI₂, YbI₃, and SmI₃.

An example of the metalloid halide includes antimony halide (for example, SbCl₅, etc.).

Examples of the metal telluride include alkali metal telluride (for example, Li₂Te, Na₂Te, K₂Te, Rb₂Te, Cs₂Te, etc.), alkaline earth metal telluride (for example, BeTe, MgTe, CaTe, SrTe, BaTe, etc.), transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), post-transition metal telluride (for example, ZnTe, etc.), and lanthanide metal telluride (for example, LaTe, CeTe, PrTe, NdTe, PmTe, EuTe, GdTe, TbTe, DyTe, HoTe, ErTe, TmTe, YbTe, LuTe, etc.).

### Emission Layer in Interlayer 130

When the light-emitting device 10 is a full-color light-emitting device, the emission layer may be patterned into a red emission layer, a green emission layer, and/or a blue emission layer, according to a sub-pixel. In one or more embodiments, the emission layer may have a stacked structure of two or more layers of a red emission layer, a green emission layer, and a blue emission layer, in which the two or more layers contact each other or are separated from each other. In one or more embodiments, the emission layer may include two or more materials of a red light-emitting material, a green light-emitting material, and a blue light-emitting material, in which the two or more materials are mixed with each other in a single layer to emit white light.

The emission layer may include a host and a dopant. The dopant may include a phosphorescent dopant, a fluorescent dopant, a delayed fluorescence dopant, or any combination thereof.

The amount of the host in the emission layer may be greater than the amount of the dopant in the emission layer. In one or more embodiments, the amount of the dopant in the emission layer may be from about 0.01 to about 15 parts by weight based on 100 parts by weight of the host.

In one or more embodiments, the emission layer may include a quantum dot.

Meanwhile, the emission layer may include a delayed fluorescence material. The delayed fluorescence material may act as a host or a dopant in an emission layer, and depending on the purpose of use, may be a delayed fluorescence host or a delayed fluorescence dopant.

A thickness of the emission layer may be in a range of about 100 Å to about 1,000 Å, for example, about 200 Å to about 600 Å. When the thickness of the emission layer is within these ranges, excellent light-emission characteristics may be obtained without a substantial increase in driving voltage.

### Host

In one or more embodiments, the host may include a compound represented by Formula 301 below:

[Ar₃₀₁]_{xb11}-[(L₃₀₁)_{xb1}-R₃₀₁]_{xb21}. Formula 301

In Formula 301,

Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xb11 may be 1, 2, or 3,

xb1 may be an integer selected from 0 to 5,

R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),

xb21 may be an integer selected from 1 to 5, and

Q₃₀₁ to Q₃₀₃ are the same as described in connection with Q₁.

For example, xb11 in Formula 301 is 2 or more, two or more of Ar₃₀₁(s) may be linked to each other via a single bond.

In one or more embodiments, the host may include a compound represented by Formula 301-1, a compound represented by Formula 301-2, or any combination thereof:

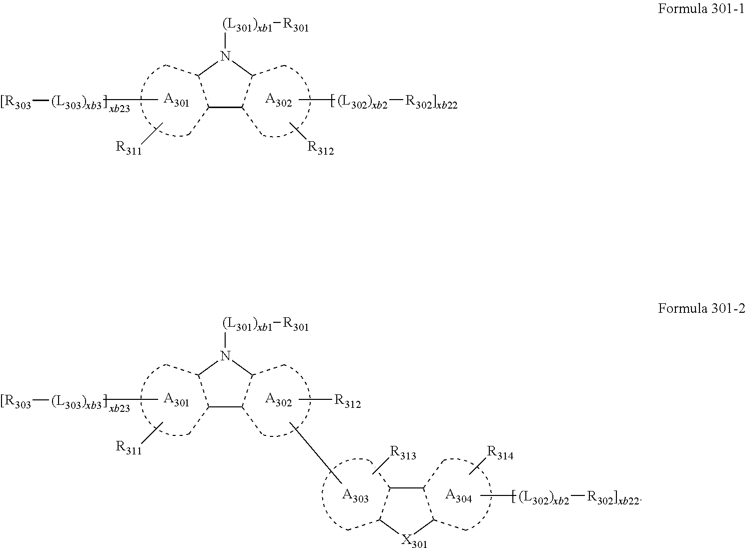

In Formulae 301-1 to 301-2,

ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

X₃₀₁ may be O, S, N-[(L₃₀₄)_{xb4}-R₃₀₄], C(R₃₀₄)(R₃₀₅), or Si(R₃₀₄)(R₃₀₅),

xb22 and xb23 may each independently be 0, 1, or 2,

L₃₀₁, xb1, and R₃₀₁ are the same as described in the present specification,

L₃₀₂ to L₃₀₄ may each independently be the same as described in connection with L₃₀₁,

xb2 to xb4 may each independently be the same as described in connection with xb1, and

R₃₀₂ to R₃₀₅ and R₃₁₁ to R₃₁₄ are the same as described in connection with R₃₀₁.

In one or more embodiments, the host may include an alkali earth metal complex, a post-transition metal complex, or a combination thereof. In one or more embodiments, the host may include a Be complex (for example, Compound H55), an Mg complex, a Zn complex, or a combination thereof.

In one or more embodiments, the host may include one of Compounds H1 to H124, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), or any combination thereof, but embodiments of the present disclosure are not limited thereto:

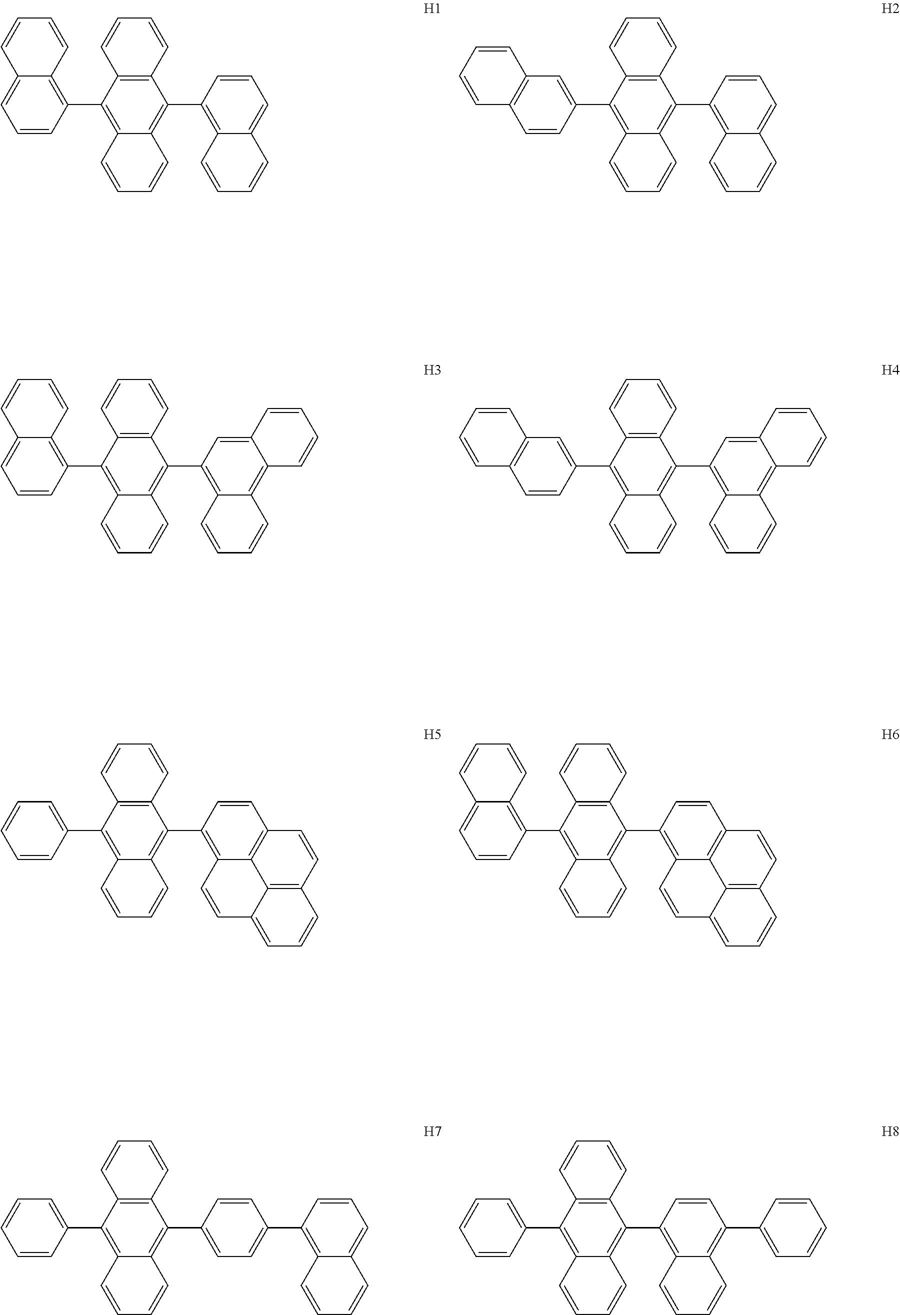

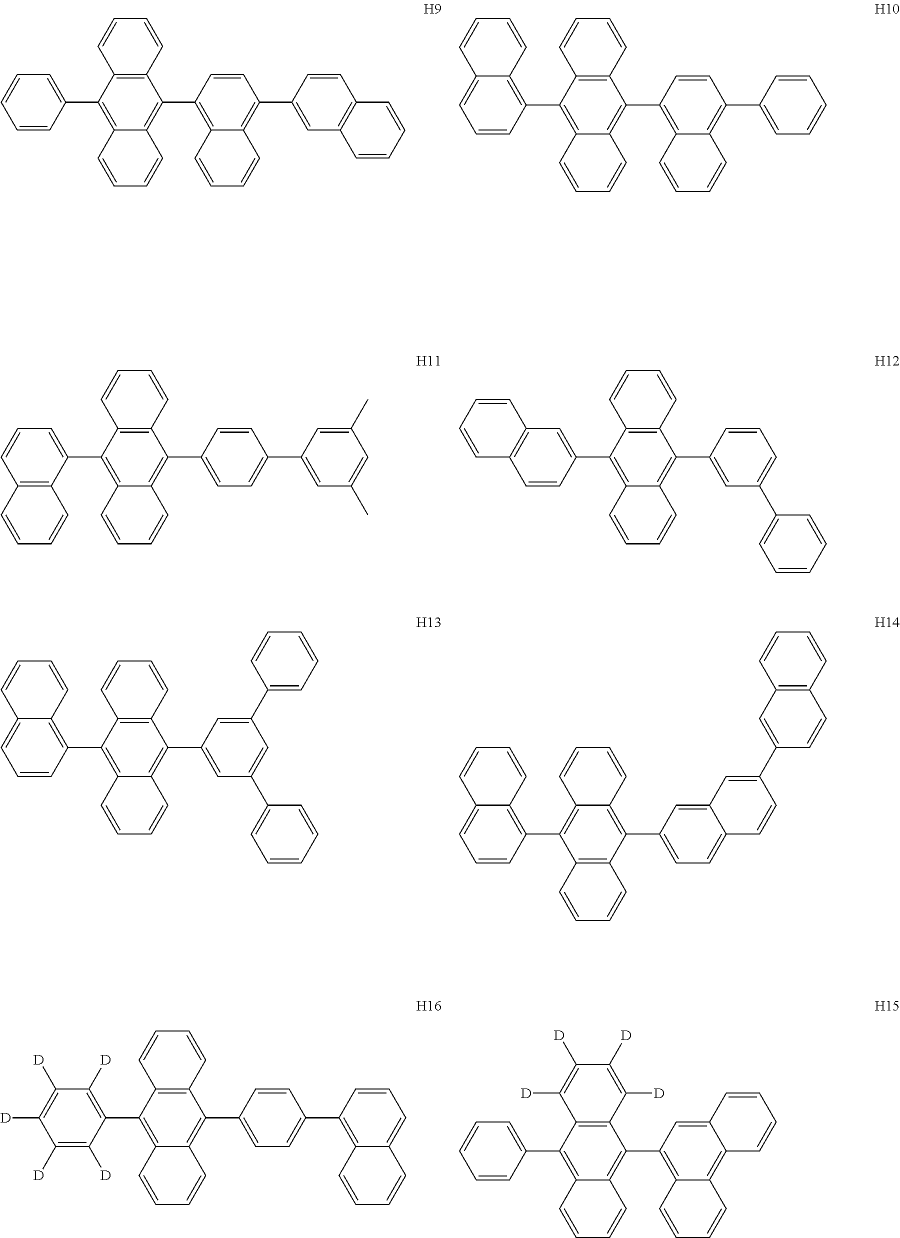

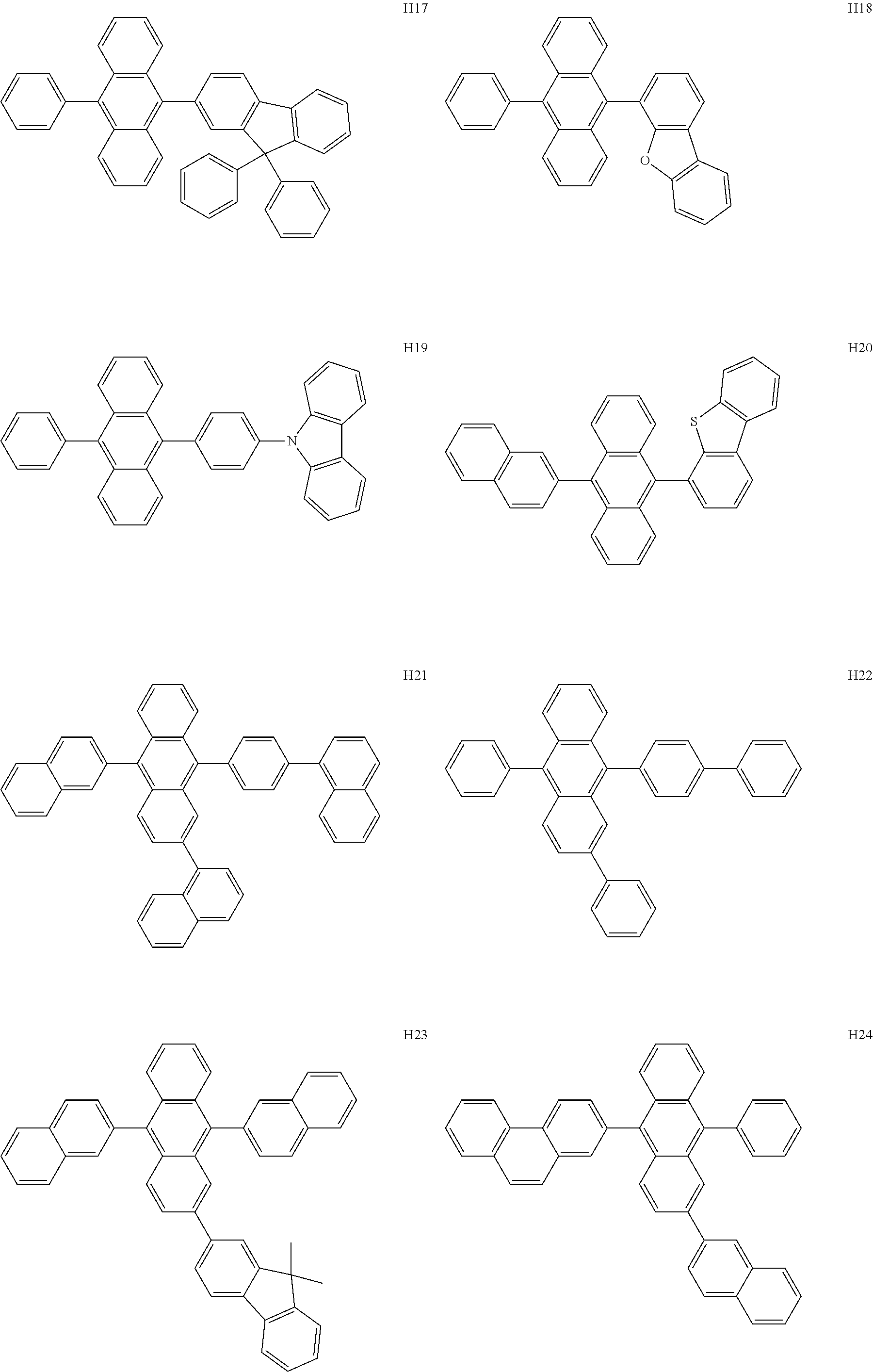

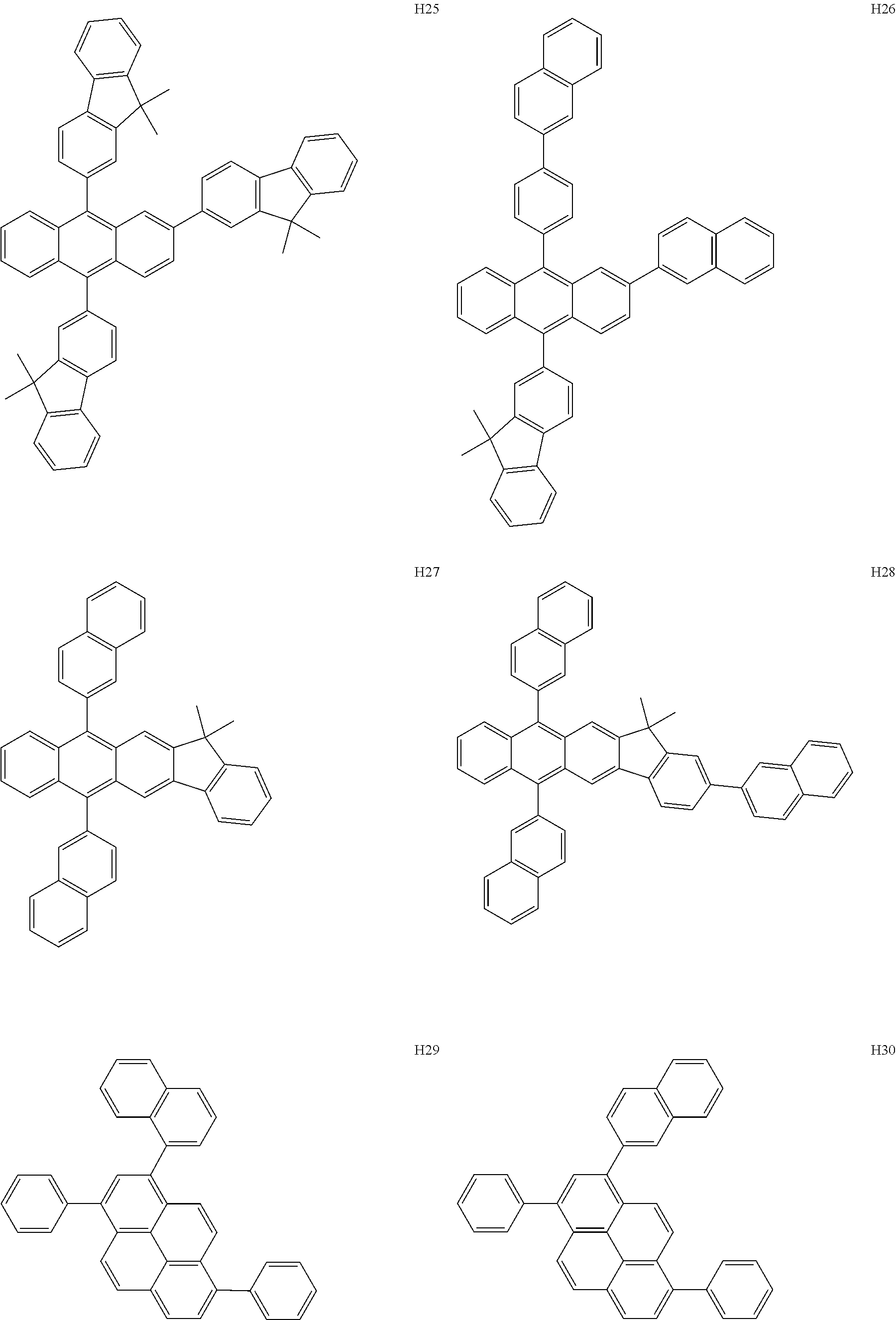

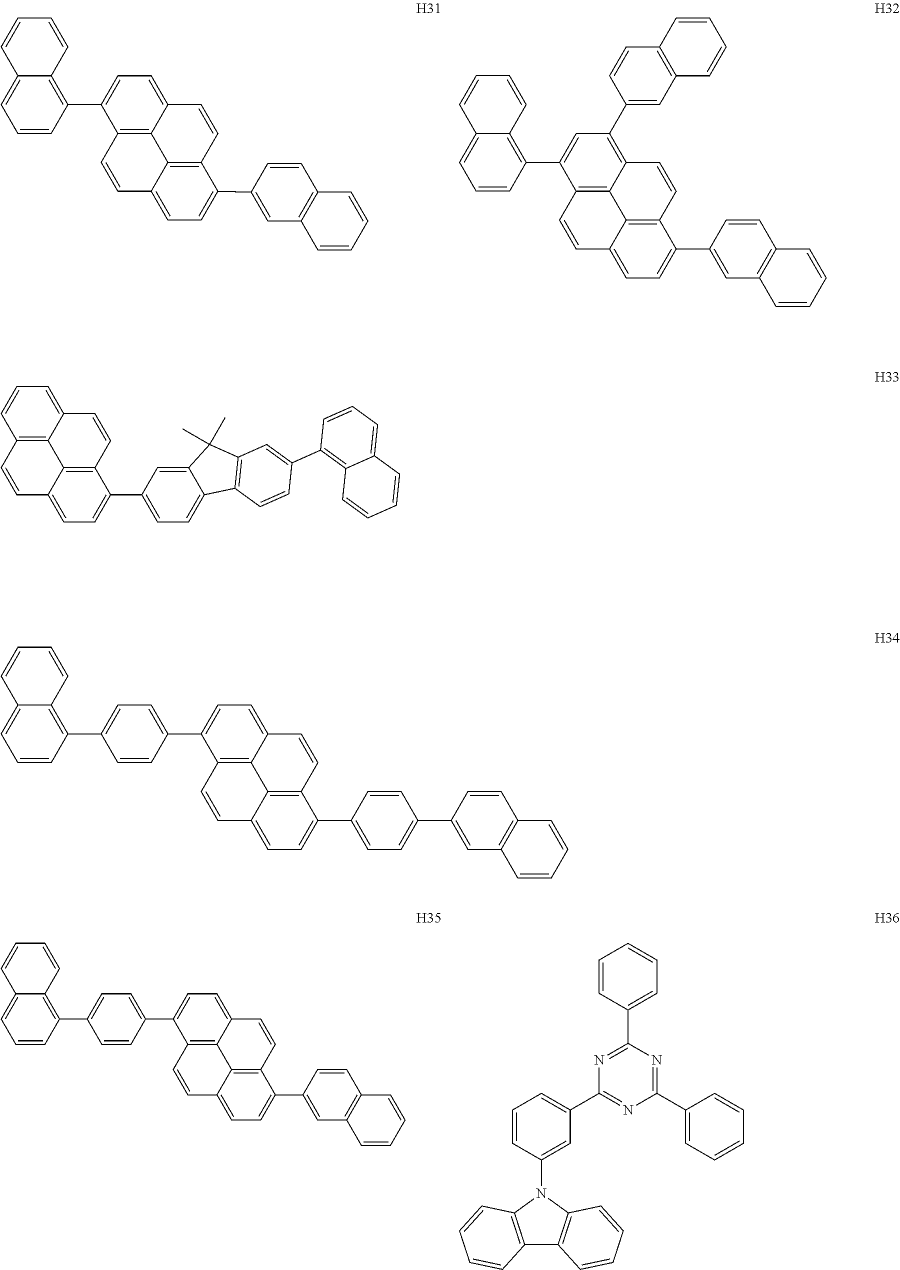

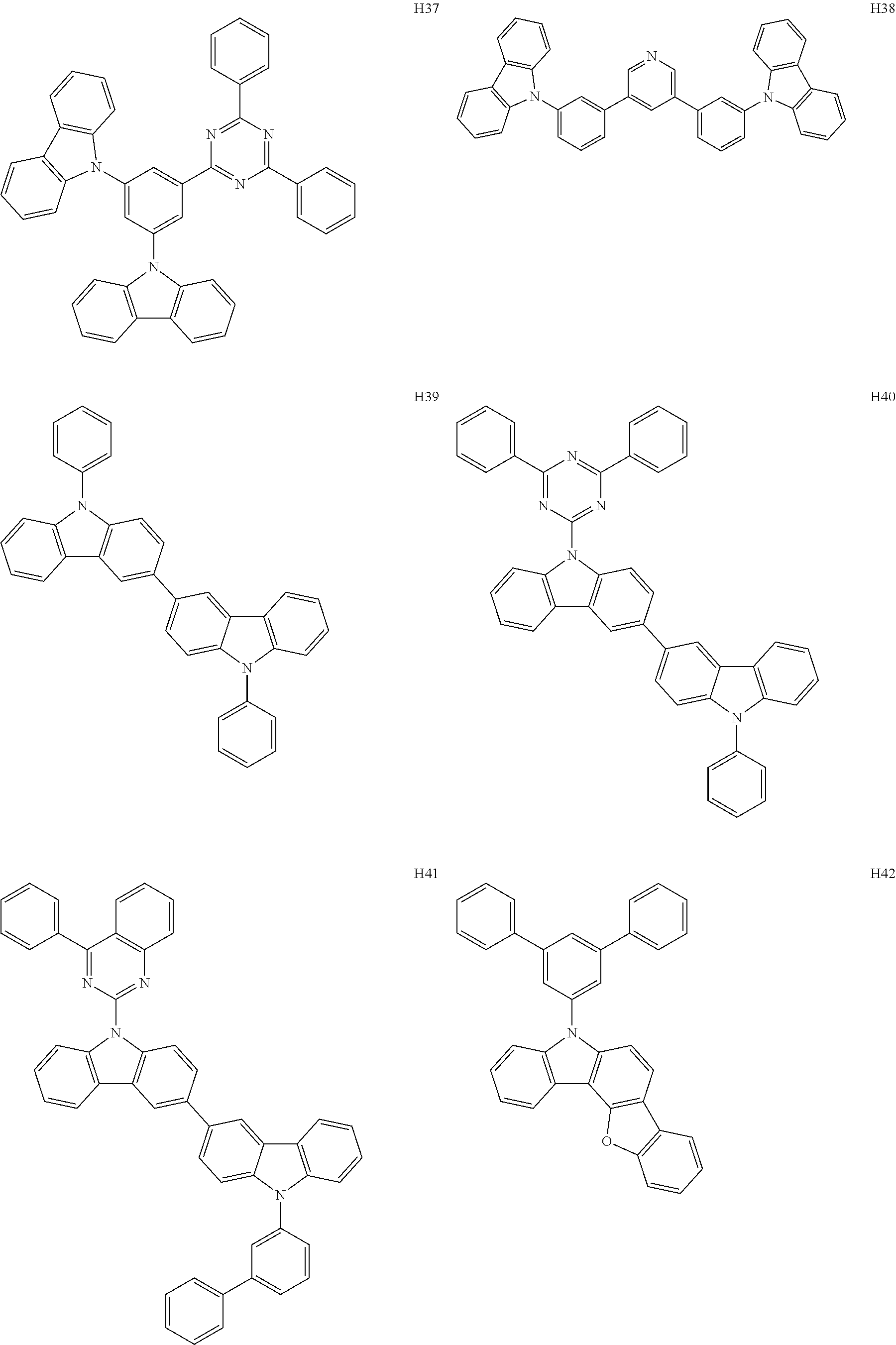 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

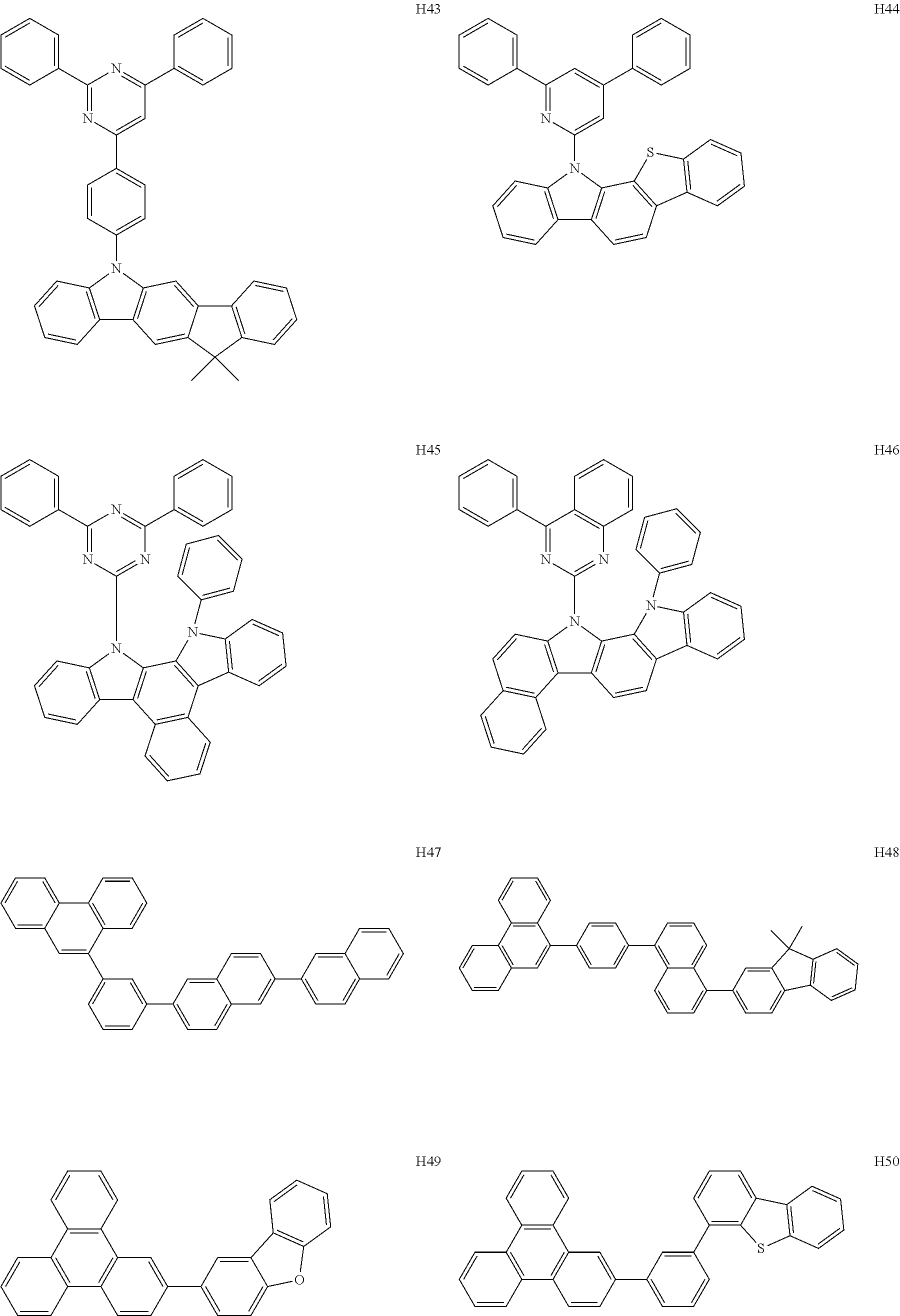

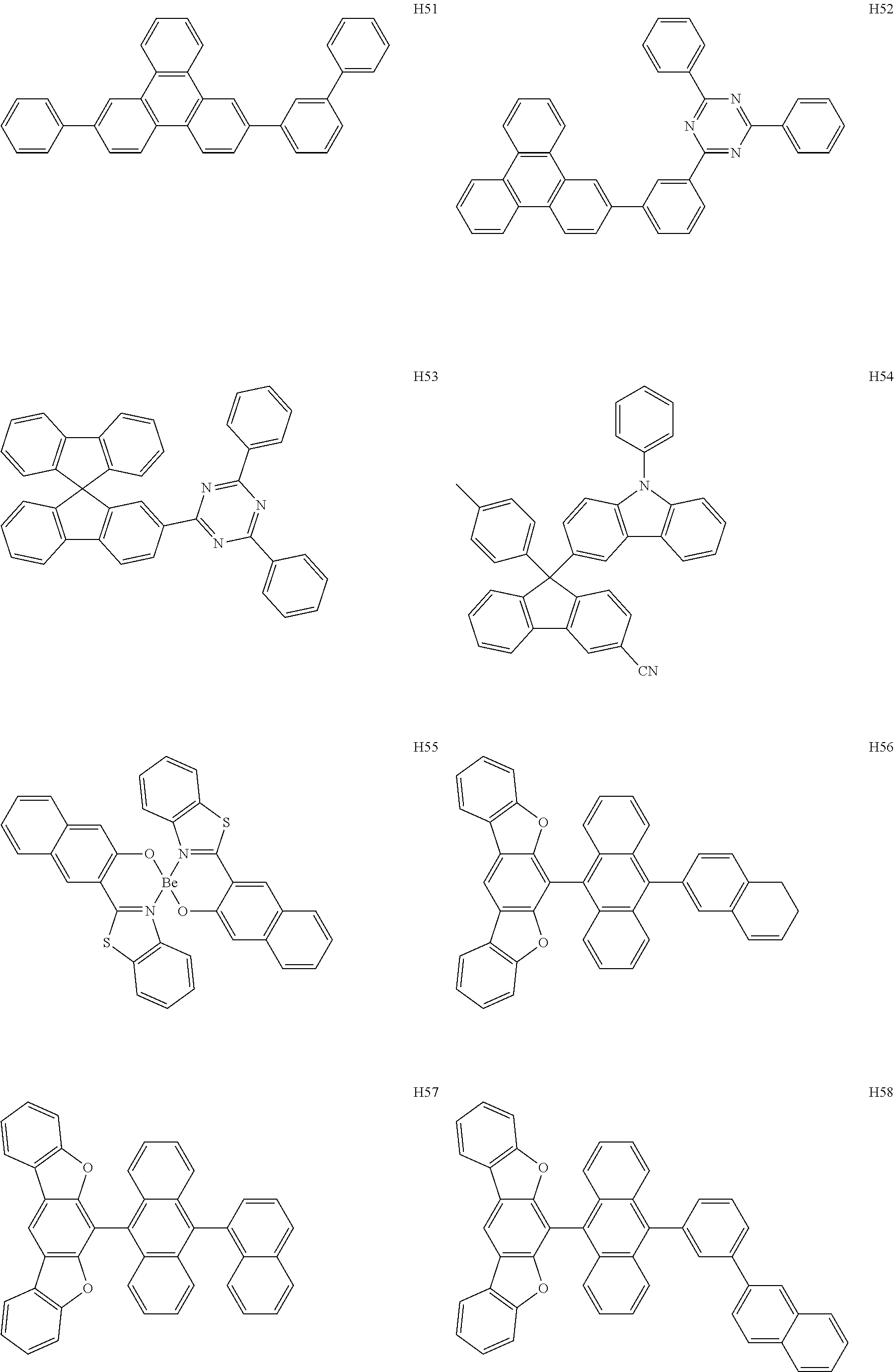

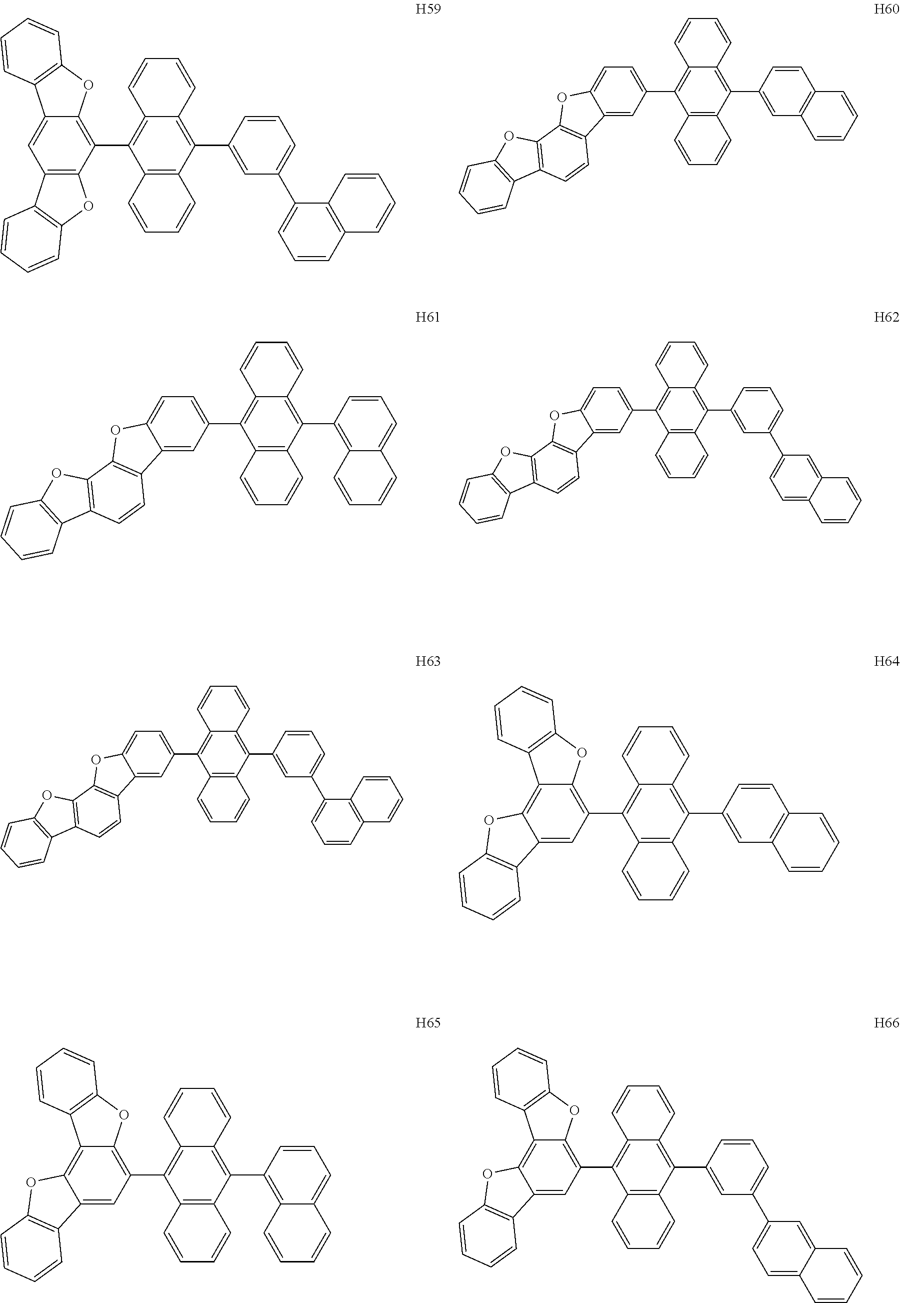

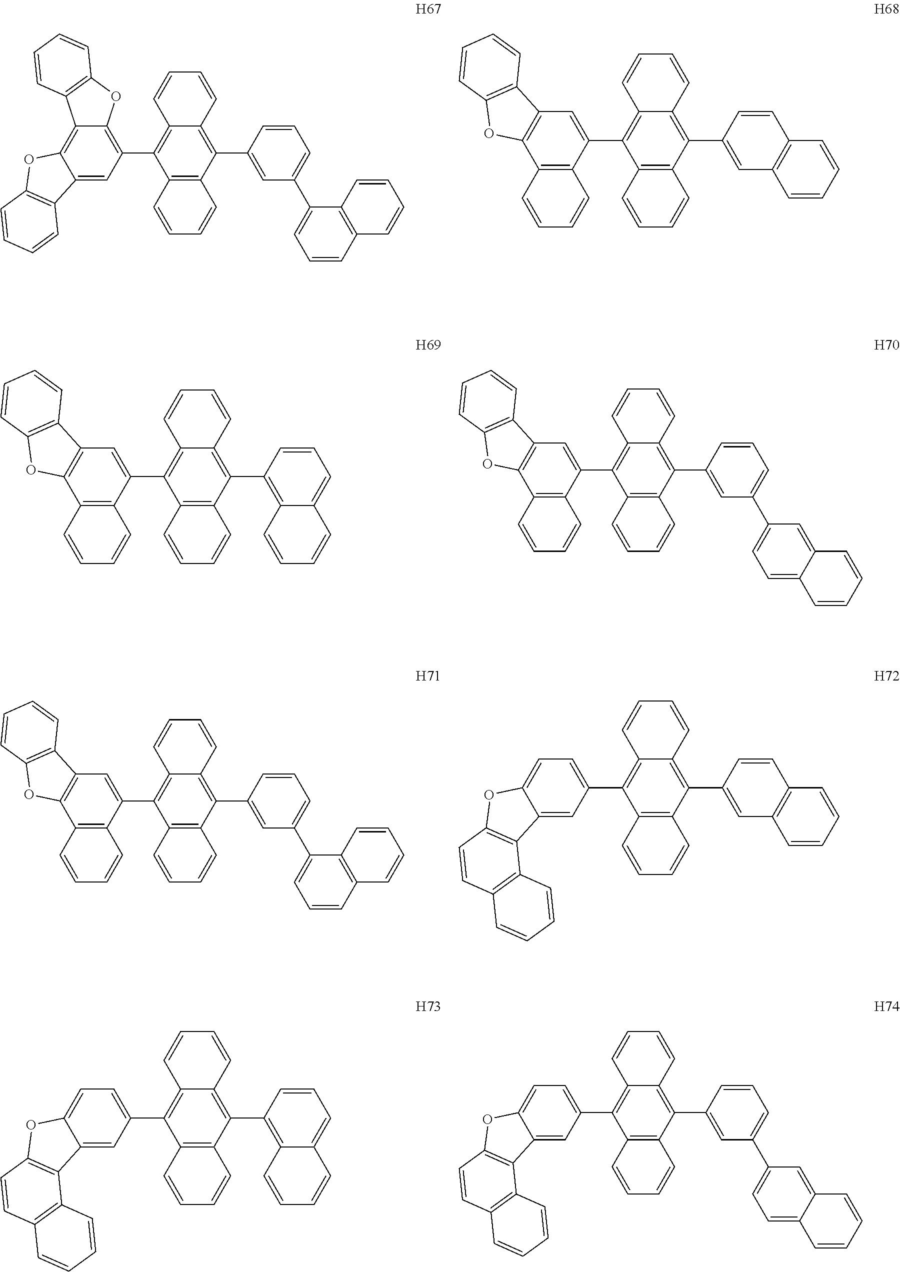

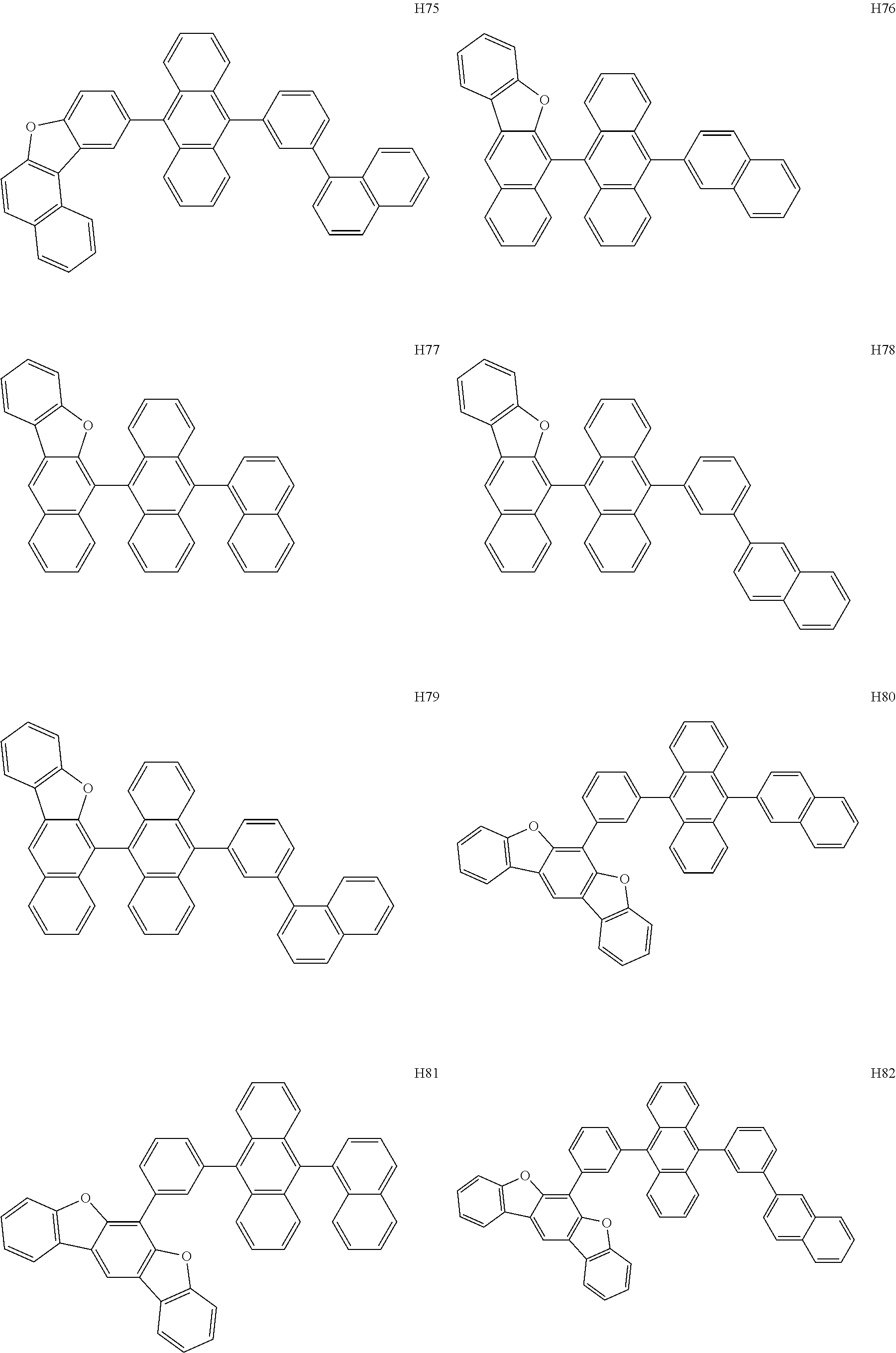

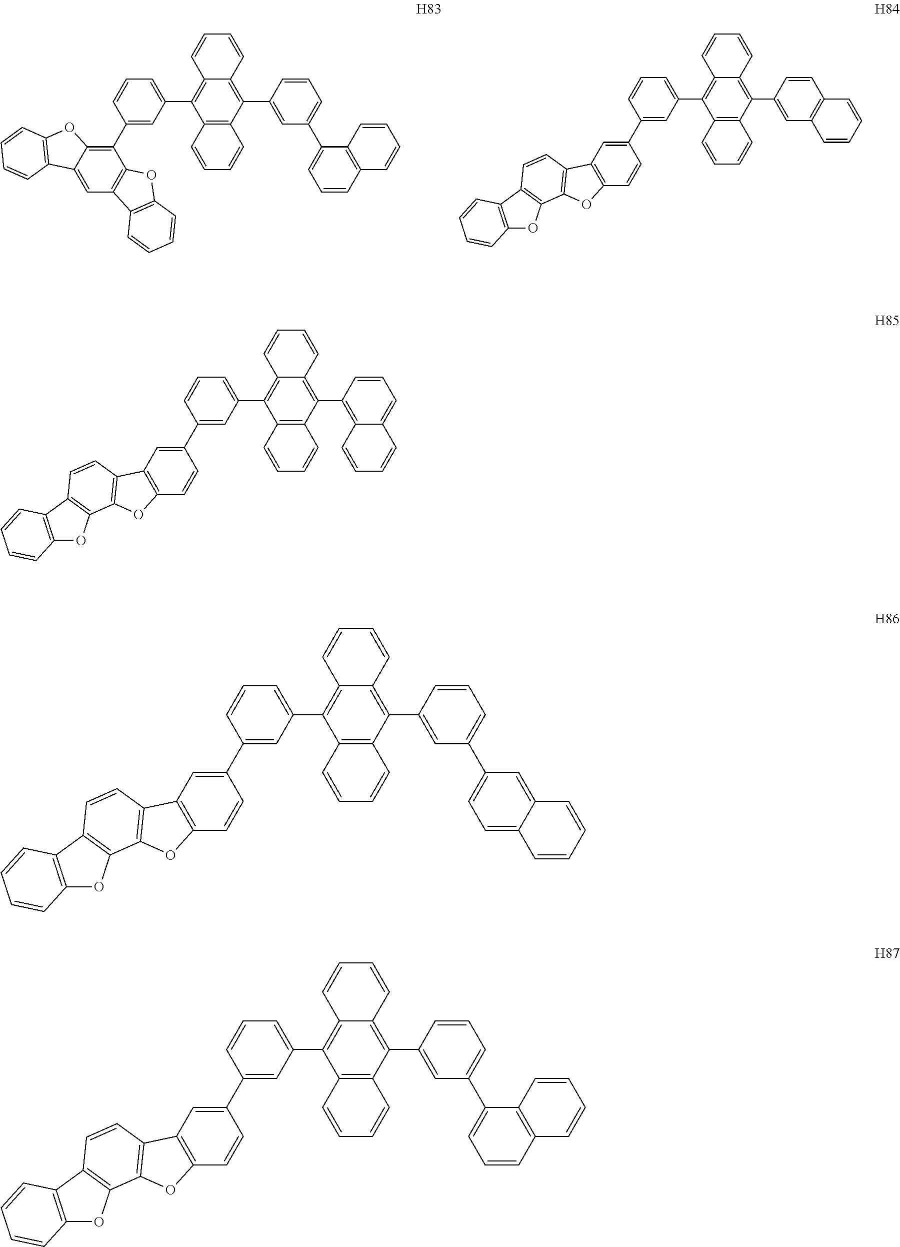 [SMILES: c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1 ; c1cc(-c2ccc3oc4c(ccc5c6ccccc6oc54)c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1 ; c1cc(-c2ccc3oc4c(ccc5c6ccccc6oc54)c3c2)cc(-c2c3ccccc3c(-c3cccc(-c4cccc5ccccc45)c3)c3ccccc23)c1]

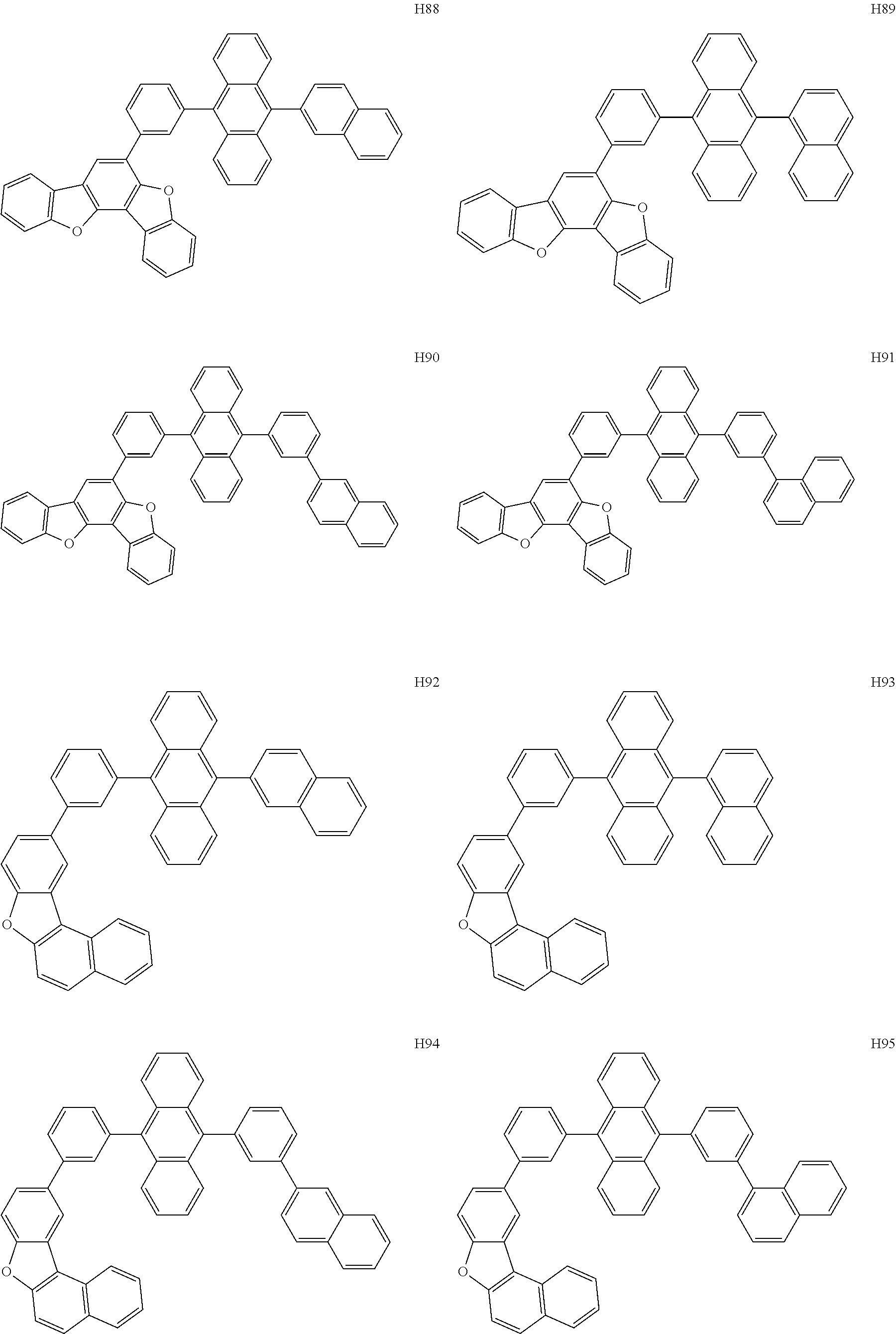

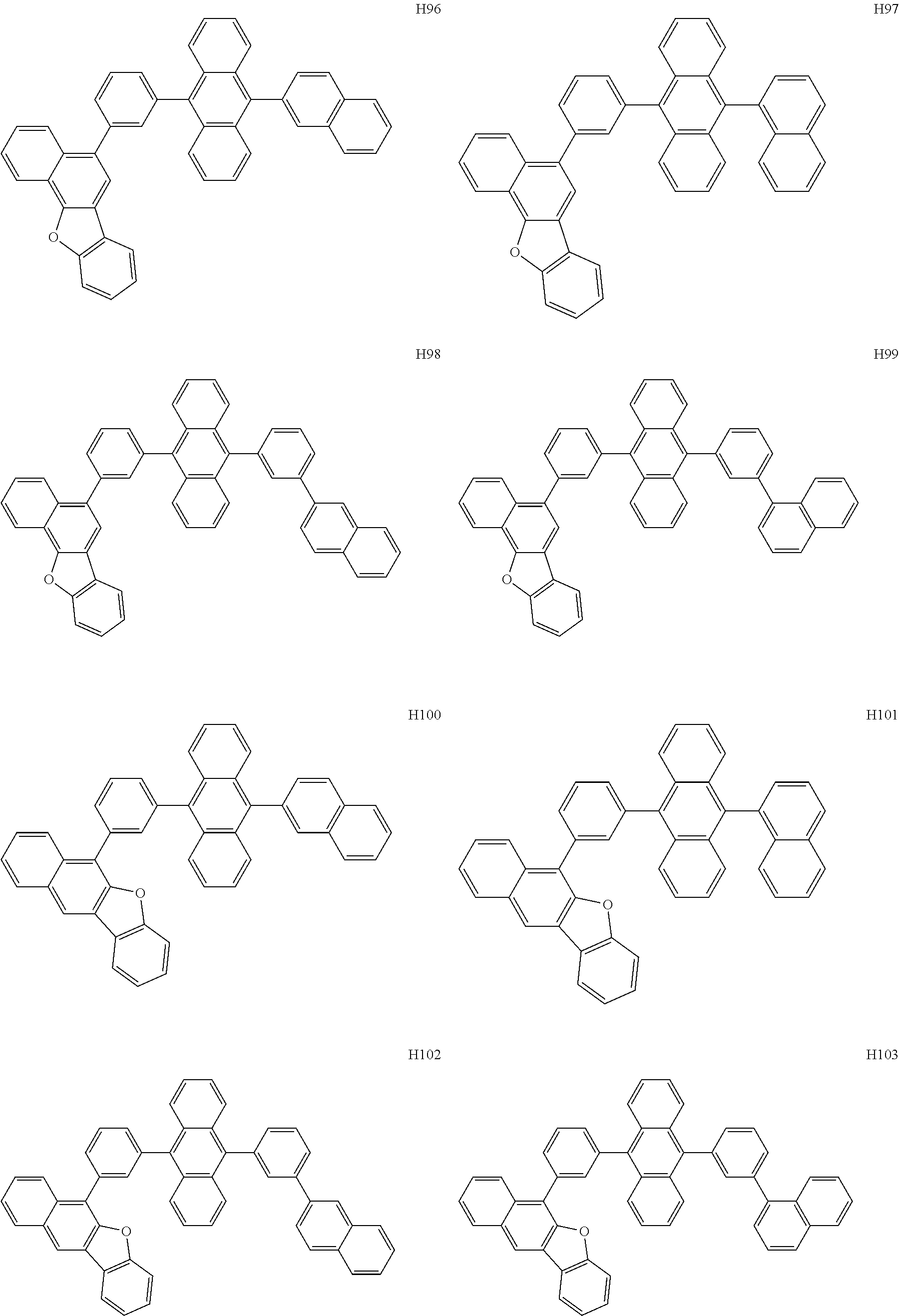

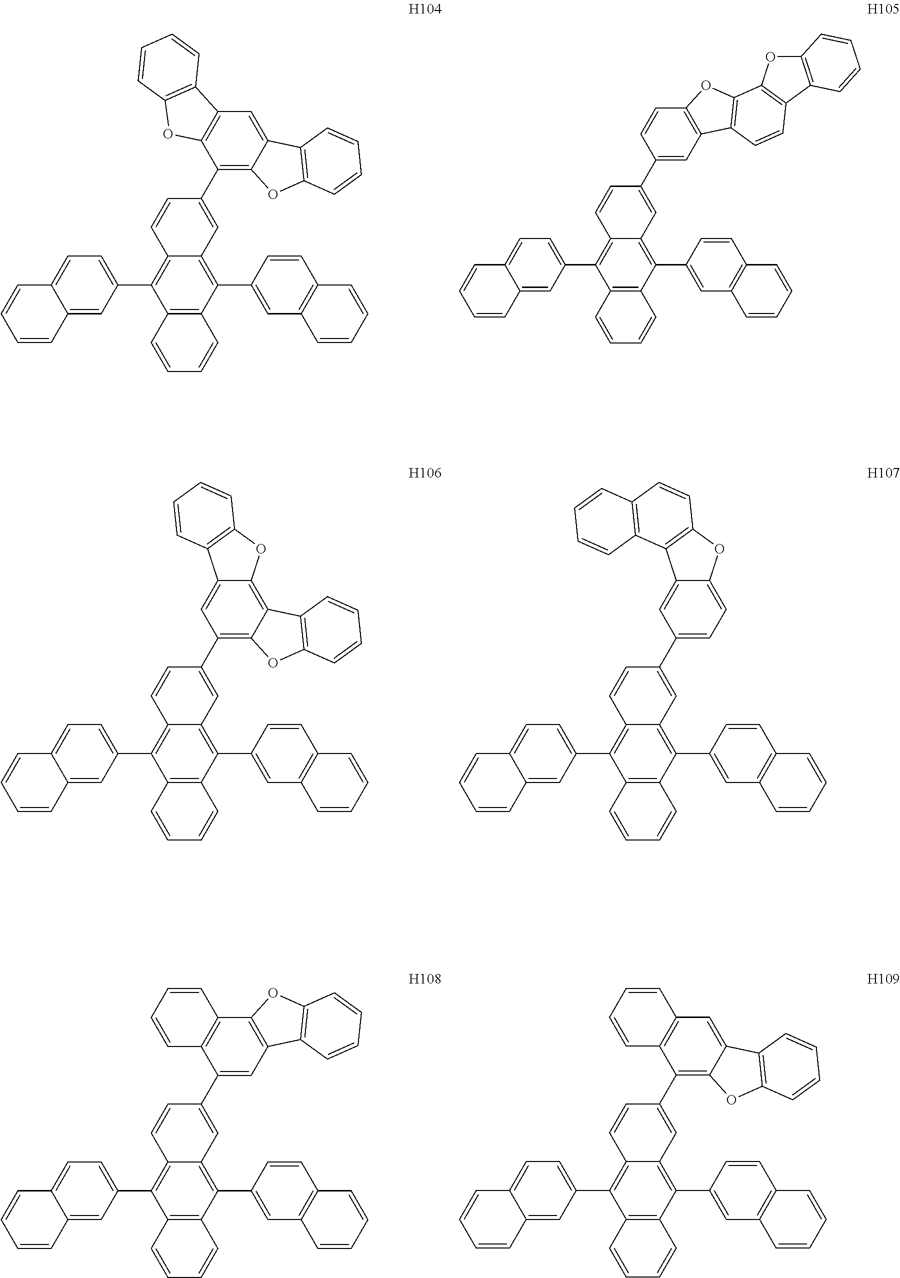

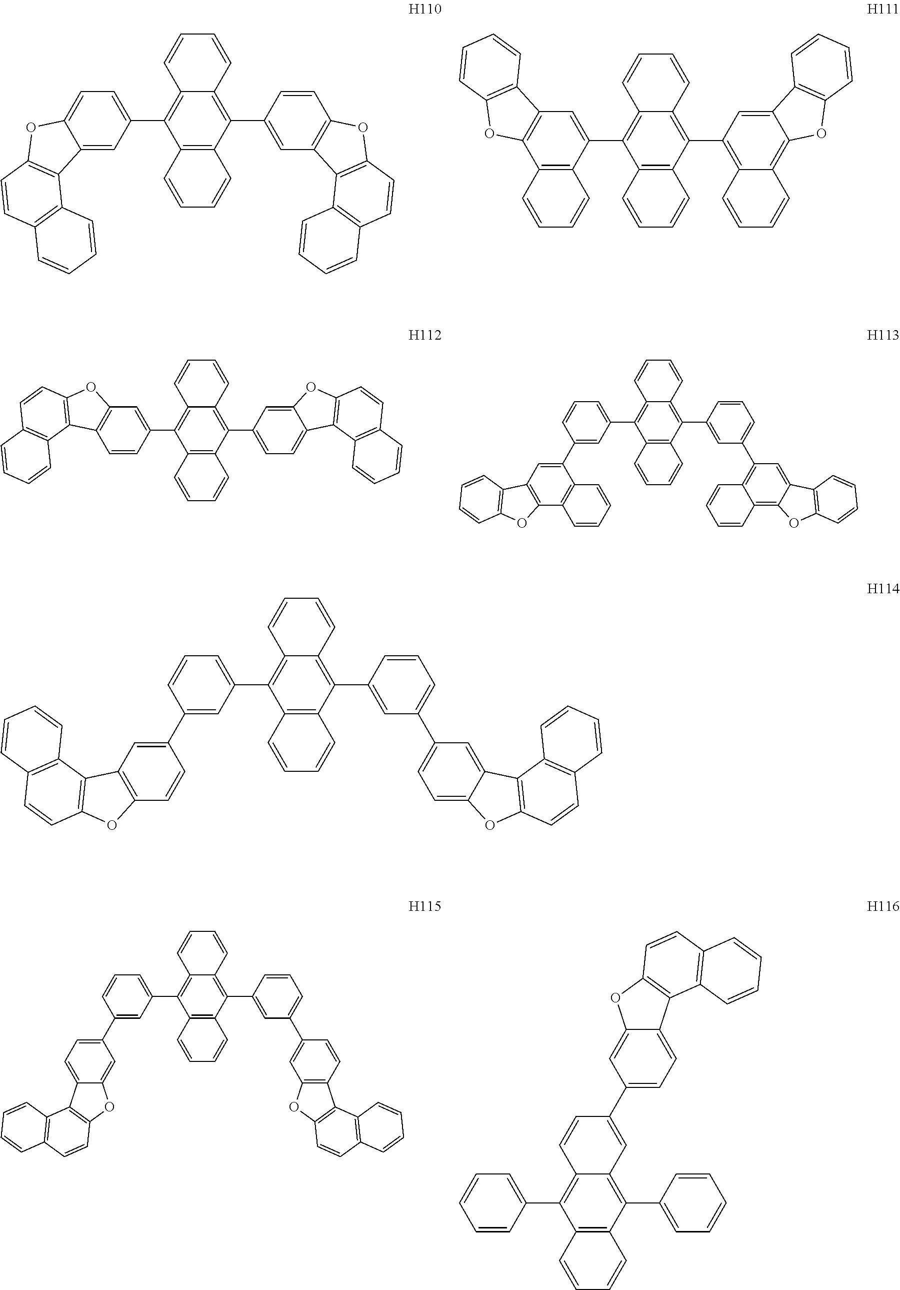

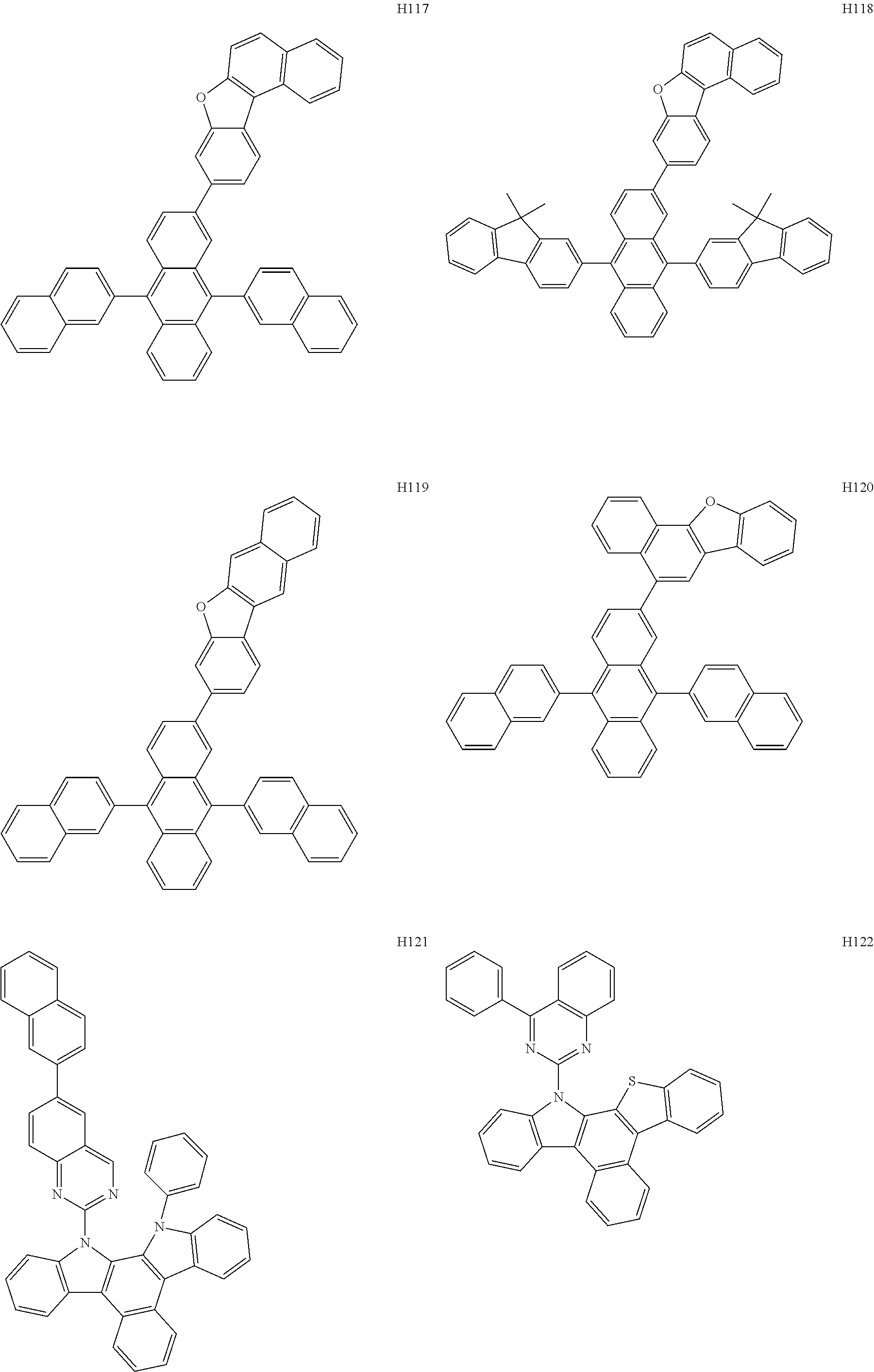

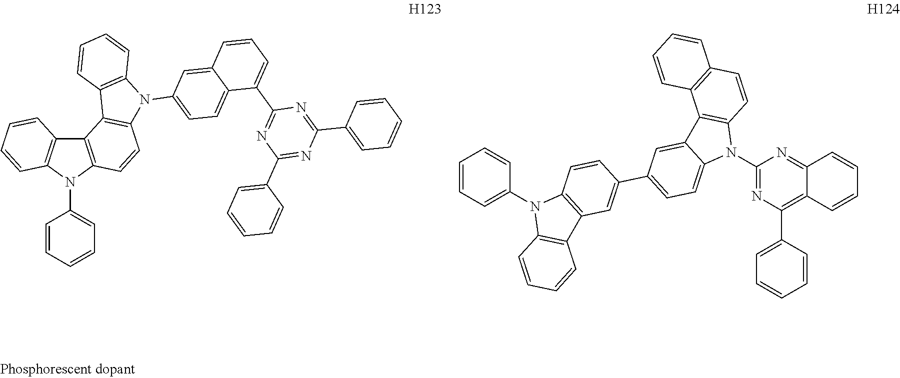 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

In one or more embodiments, the phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

For example, the phosphorescent dopant may include an organometallic compound represented by Formula 401:

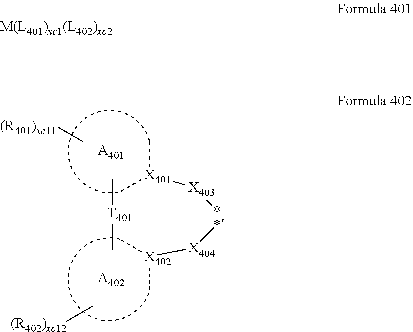

In Formulae 401 and 402,

M may be transition metal (for example, iridium (Ir), platinum (Pt), palladium (Pd), osmium (Os), titanium (Ti), gold (Au)hafnium (Hf), europium (Eu), terbium (Tb), rhodium (Rh), rhenium (Re), or thulium (Tm)),

L₄₀₁ may be a ligand represented by Formula 402, and xc1 may be 1, 2, or 3, wherein when xc1 is two or more, two or more of L₄₀₁(s) may be identical to or different from each other,

L₄₀₂ may be an organic ligand, and xc2 may be 0, 1, 2, 3, or 4, and when xc2 is 2 or more, two or more of L₄₀₂(s) may be identical to or different from each other,

X₄₀₁ and X₄₀₂ may each independently be nitrogen or carbon,

ring A₄₀₁ and ring A₄₀₂ may each independently be a C₃-C₆₀ carbocyclic group or a C₁-C₆₀ heterocyclic group,

T₄₀₁ may be a single bond, *—O—*′, *—S—*′, *—C(═O)—*′, *—N(Q₄₁₁)—*′, *—C(Q₄₁₁)(Q₄₁₂)—*′ *—C(Q₄₁₁)═C(Q₄₁₂)—*′, *—C(Q₄₁₁)=*′, or *═C(Q₄₁₁)=*′,

X₄₀₃ and X₄₀₄ may each independently be a chemical bond (for example, a covalent bond or a coordination bond), O, S, N(Q₄₁₃), B(Q₄₁₃), P(Q₄₁₃), C(Q₄₁₃)(Q₄₁₄), or Si(Q₄₁₃)(Q₄₁₄),

Q₄₁₁ to Q₄₁₄ are the same as described in connection with Q₁,

R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),

Q₄₀₁ to Q₄₀₃ are the same as described in connection with Q₁,

xc11 and xc12 may each independently be an integer selected from 0 to 10,

* and *′ in Formula 402 each indicate a binding site to M in Formula 401.

For example, in Formula 402, i) X₄₀₁ is nitrogen, and X₄₀₂ is carbon, or ii) each of X₄₀₁ and X₄₀₂ is nitrogen.

In one or more embodiments, when xc1 in Formula 402 is 2 or more, two ring A₄₀₁ in two or more of L₄₀₁(s) may be optionally linked to each other via T₄₀₂, which is a linking group, and two ring A₄₀₂ may optionally be linked to each other via T₄₀₃, which is a linking group (see Compounds PD1 to PD4 and PD7). T₄₀₂ and T₄₀₃ are the same as described in connection with T₄₀₁.

L₄₀₂ in Formula 401 may be an organic ligand. For example, L₄₀₂ may include a halogen group, a diketone group (for example, an acetylacetonate group), a carboxylic acid group (for example, a picolinate group), —C(═O), an isonitrile group, —CN group, a phosphorous group (for example, a phosphine group, a phosphite group, etc.), or any combination thereof.

The phosphorescent dopant may include, for example, one of compounds PD1 to PD25, or any combination thereof:

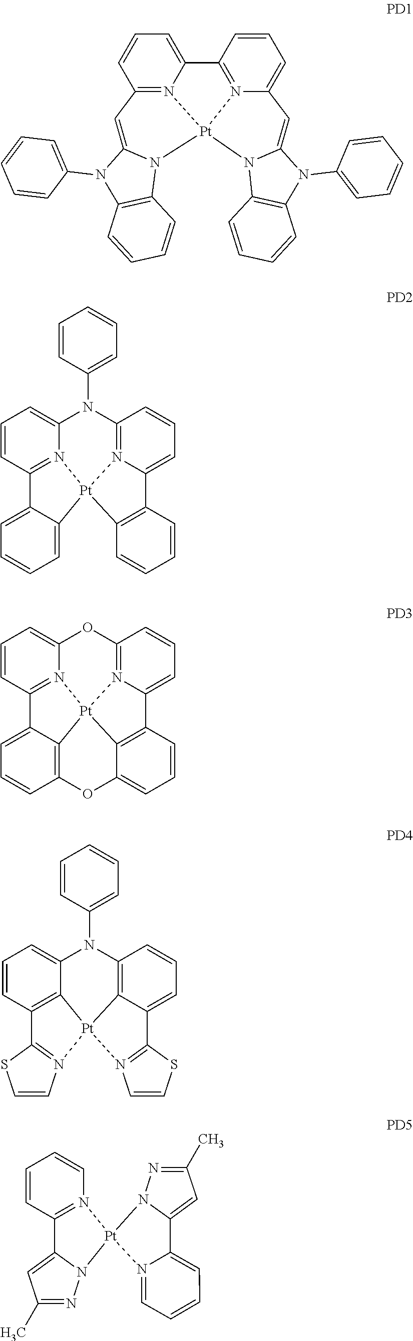

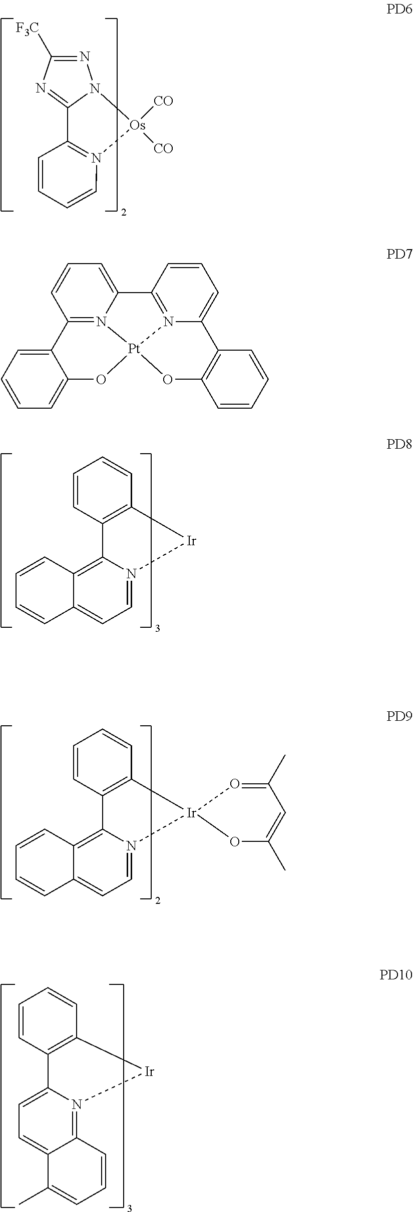

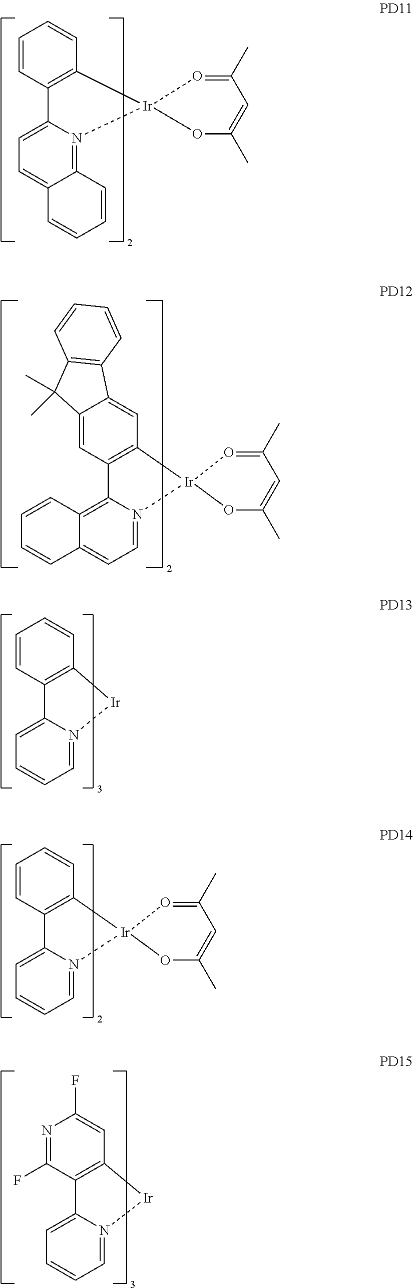

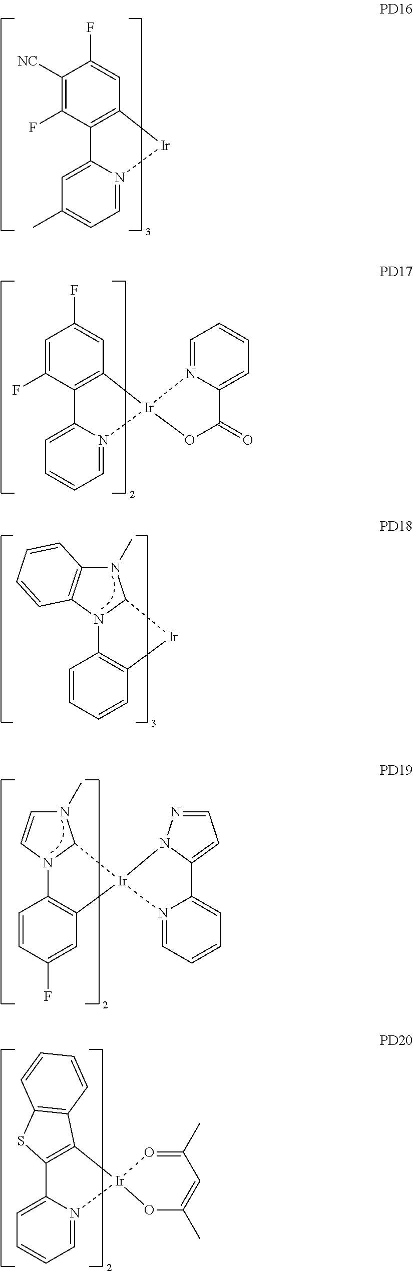

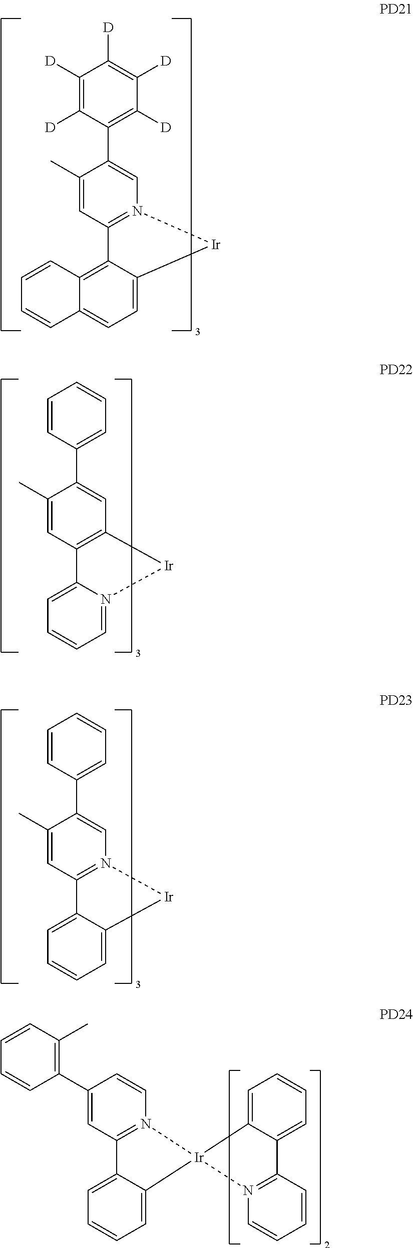

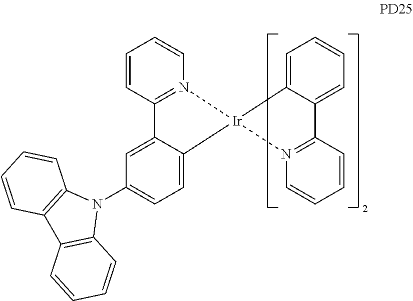

### Fluorescent Dopant

The fluorescent dopant may include an amine group-containing compound, a styryl group-containing compound, or any combination thereof.

In one or more embodiments, the fluorescent dopant may include a compound represented by Formula 501:

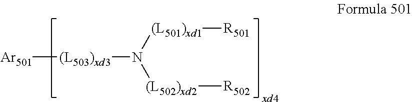

wherein, in Formula 501,

Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xd1 to xd3 may each independently be 0, 1, 2, or 3,

xd4 may be 1, 2, 3, 4, 5, or 6.

In one or more embodiments, Ar₅₀₁ in Formula 501 may be a condensed cyclic group (for example, an anthracene group, a chrysene group, or a pyrene group) in which three or more monocyclic groups are condensed together.

In one or more embodiments, xd4 in Formula 501 may be 2.

In one or more embodiments, the fluorescent dopant may include: one of Compounds FD1 to FD36; DPVBi; DPAVBi; or any combination thereof:

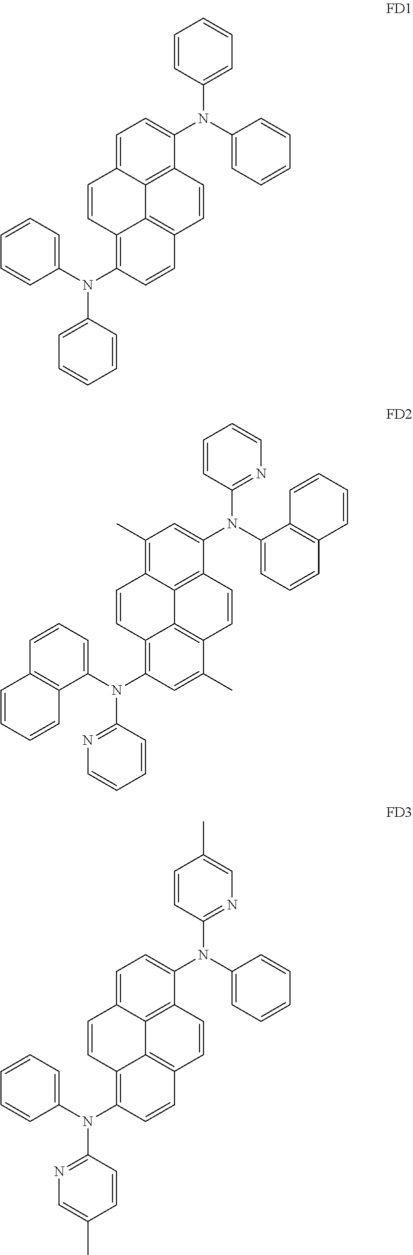 [SMILES: c1ccc(N(c2ccccc2)c2ccc3ccc4c(N(c5ccccc5)c5ccccc5)ccc5ccc2c3c54)cc1 ; Cc1cc(N(c2ccccn2)c2cccc3ccccc23)c2ccc3c(C)cc(N(c4ccccn4)c4cccc5ccccc45)c4ccc1c2c34 ; Cc1ccc(N(c2ccccc2)c2ccc3ccc4c(N(c5ccccc5)c5ccc(C)cn5)ccc5ccc2c3c54)nc1]

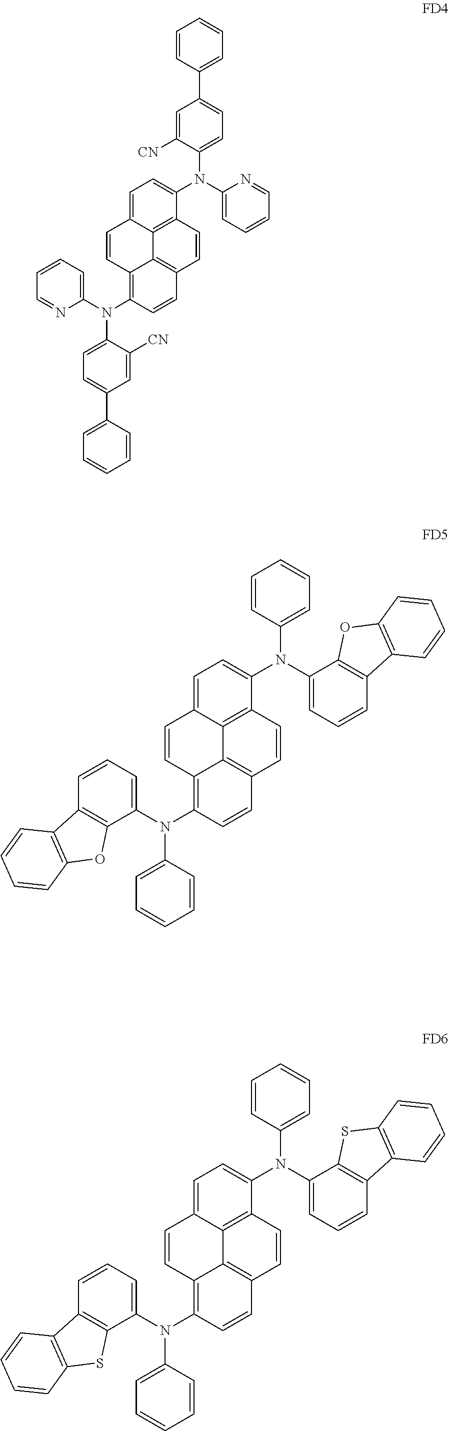

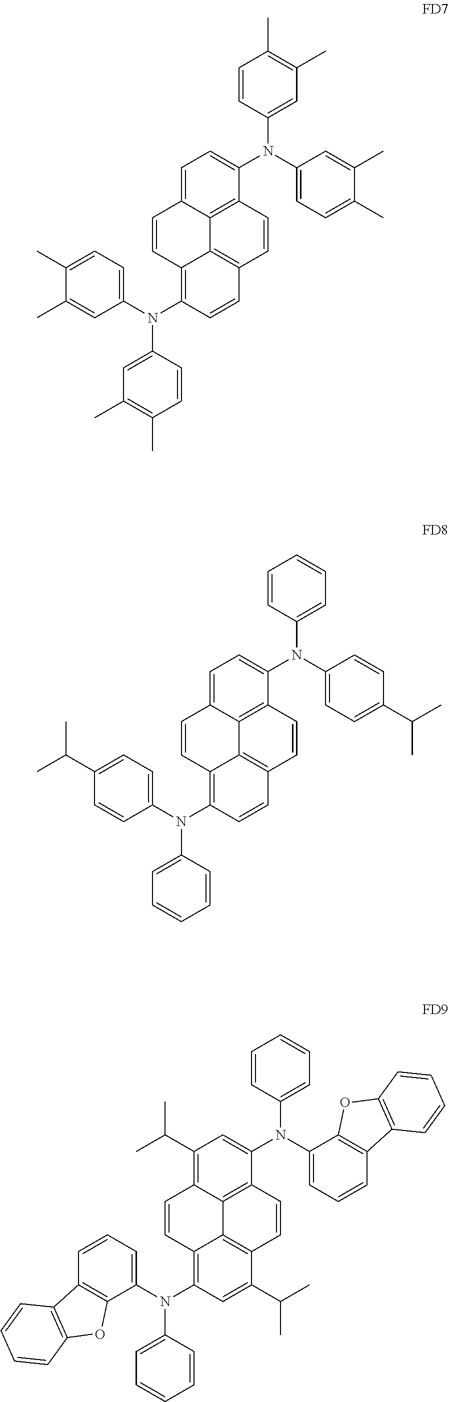

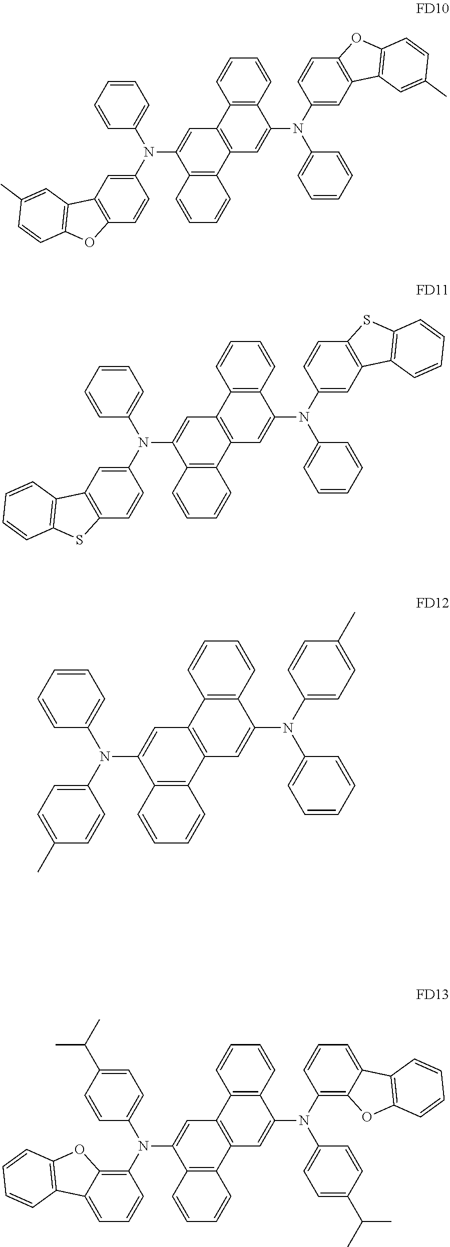

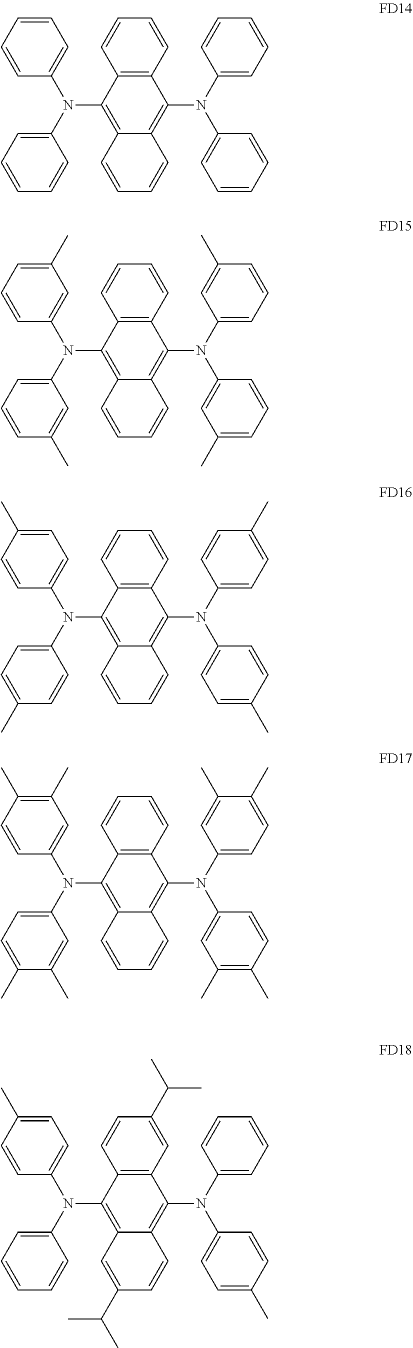

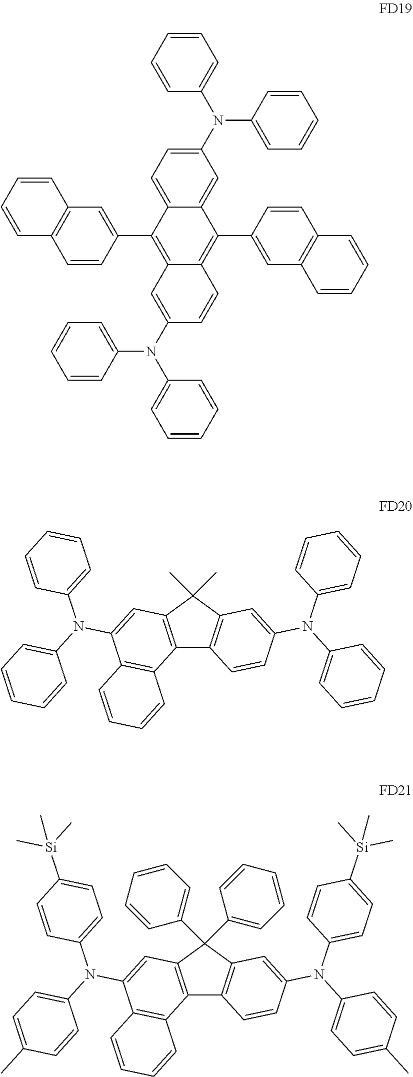

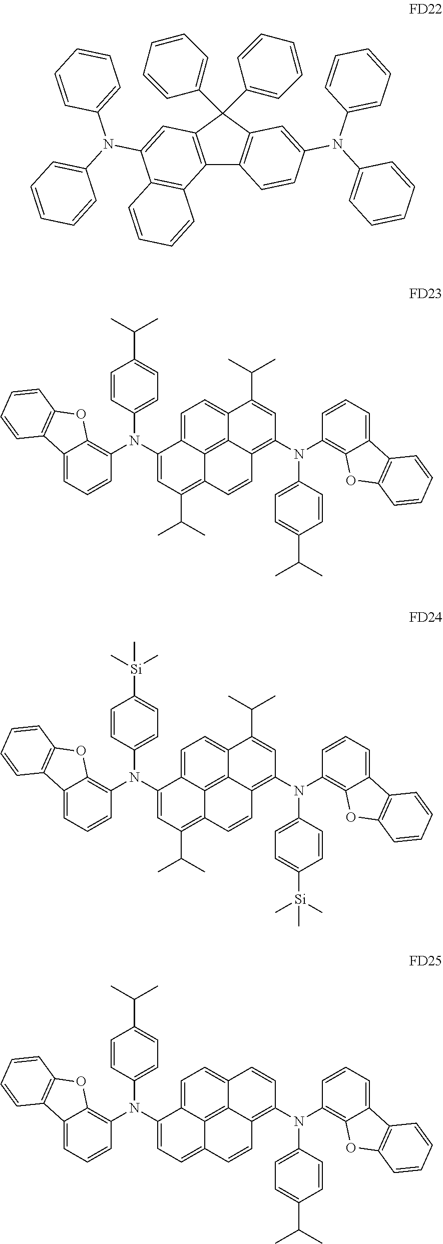 [SMILES: c1ccc(N(c2ccccc2)c2ccc3c(c2)C(c2ccccc2)(c2ccccc2)c2cc(N(c4ccccc4)c4ccccc4)c4ccccc4c2-3)cc1 ; CC(C)c1ccc(N(c2cc(C(C)C)c3ccc4c(N(c5ccc(C(C)C)cc5)c5cccc6c5oc5ccccc56)cc(C(C)C)c5ccc2c3c54)c2cccc3c2oc2ccccc23)cc1 ; CC(C)c1cc(N(c2ccc([Si](C)(C)C)cc2)c2cccc3c2oc2ccccc23)c2ccc3c(C(C)C)cc(N(c4ccc([Si](C)(C)C)cc4)c4cccc5c4oc4ccccc45)c4ccc1c2c34 ; CC(C)c1ccc(N(c2ccc3ccc4c(N(c5ccc(C(C)C)cc5)c5cccc6c5oc5ccccc56)ccc5ccc2c3c54)c2cccc3c2oc2ccccc23)cc1]

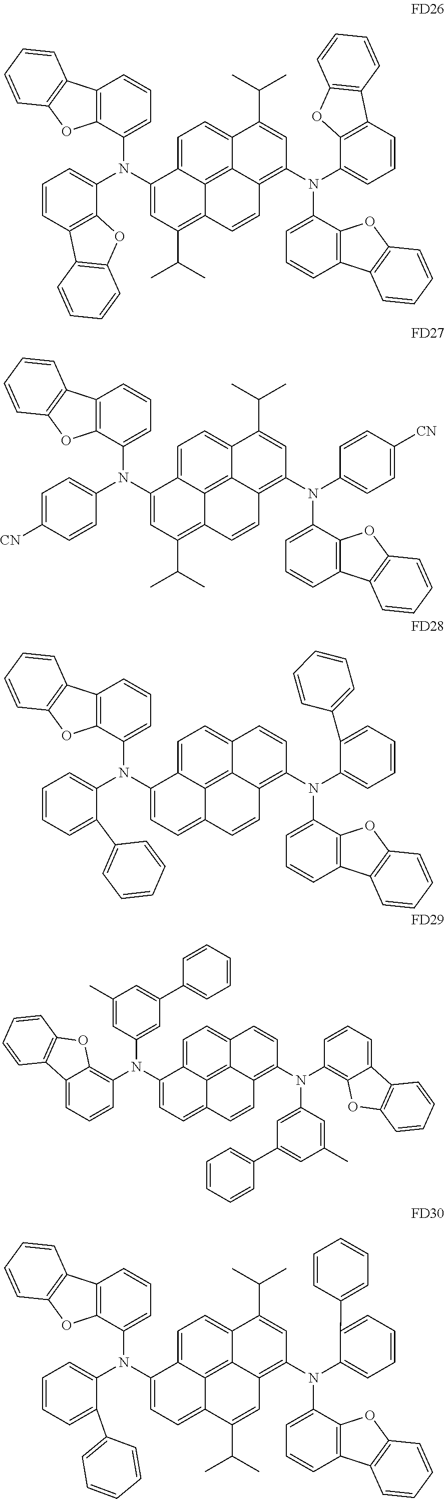

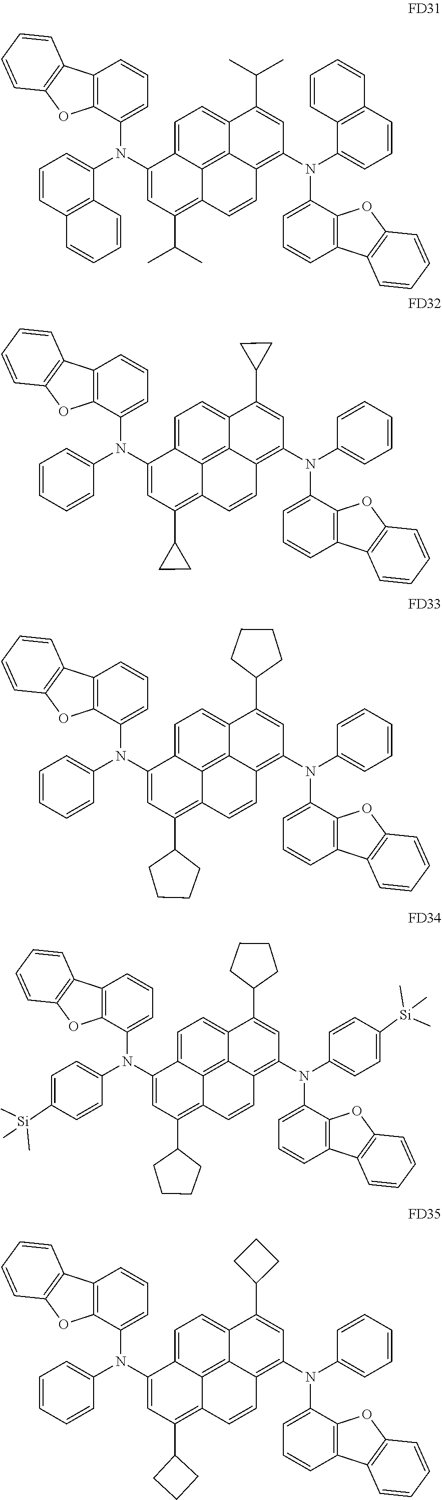

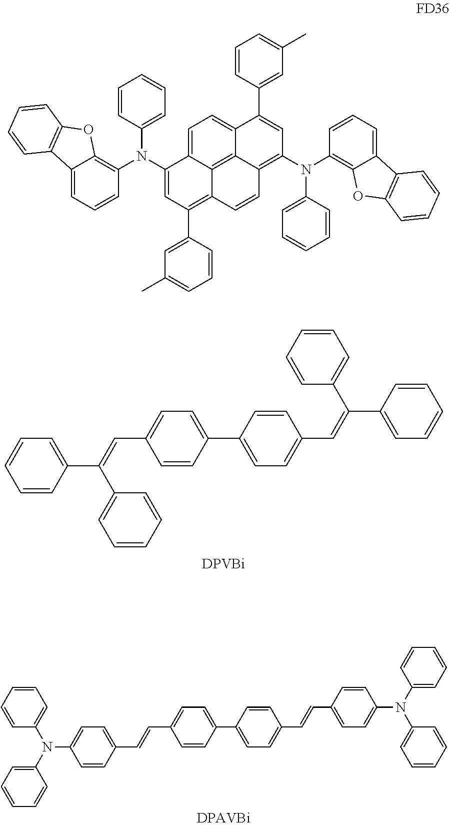

### Delayed Fluorescence Material

Meanwhile, the emission layer may include a delayed fluorescence material.

In the present specification, the delayed fluorescence material may be selected from compounds capable of emitting delayed fluorescence based on a delayed fluorescence emission mechanism.

The delayed fluorescent material included in the emission layer may act as a host or a dopant depending on the type of other materials included in the emission layer.

In one or more embodiments, the difference between the triplet energy level (eV) of the delayed fluorescence material and the singlet energy level (eV) of the delayed fluorescence material may be greater than or equal to 0 eV and less than or equal to 0.5 eV. When the difference between the triplet energy level (eV) of the delayed fluorescent material and the singlet energy level (eV) of the delayed fluorescent material satisfies the above-described range, up-conversion from the triplet state to the singlet state of the delayed fluorescent materials may effectively occur, and thus, the emission efficiency of the light-emitting device 10 may be improved.

In one or more embodiments, the delayed fluorescence material may include i) a material including at least one electron donor (for example, a π electron-rich C₃-C₆₀ cyclic group, such as a carbazole group) and at least one electron acceptor (for example, a sulfoxide group, a cyano group, or a π electron-deficient nitrogen-containing C₁-C₆₀ cyclic group), and ii) a material including a C₈-C₆₀ polycyclic group in which two or more cyclic groups are condensed while sharing boron (B).

In one or more embodiments, the delayed fluorescence material may include at least one of the following compounds DF1 to DF9:

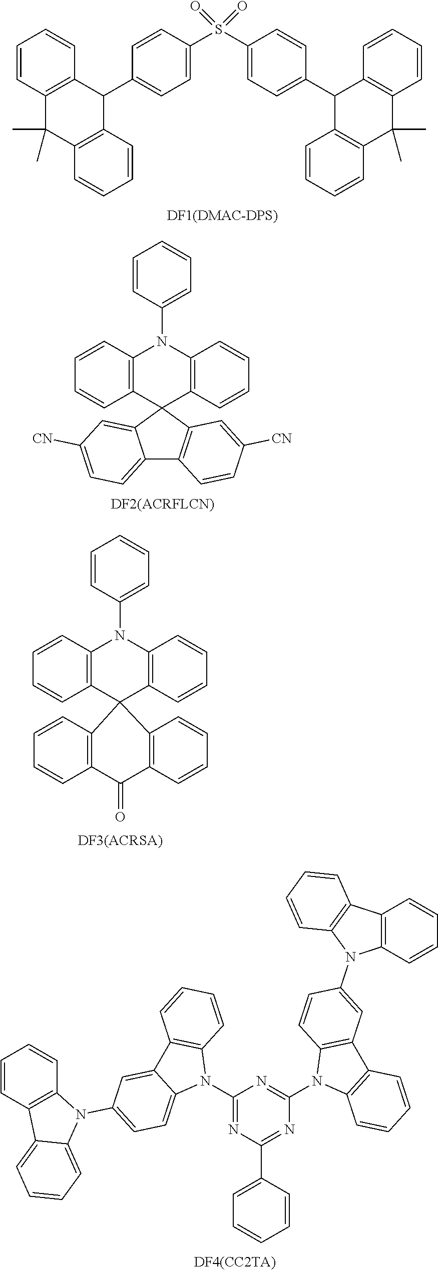

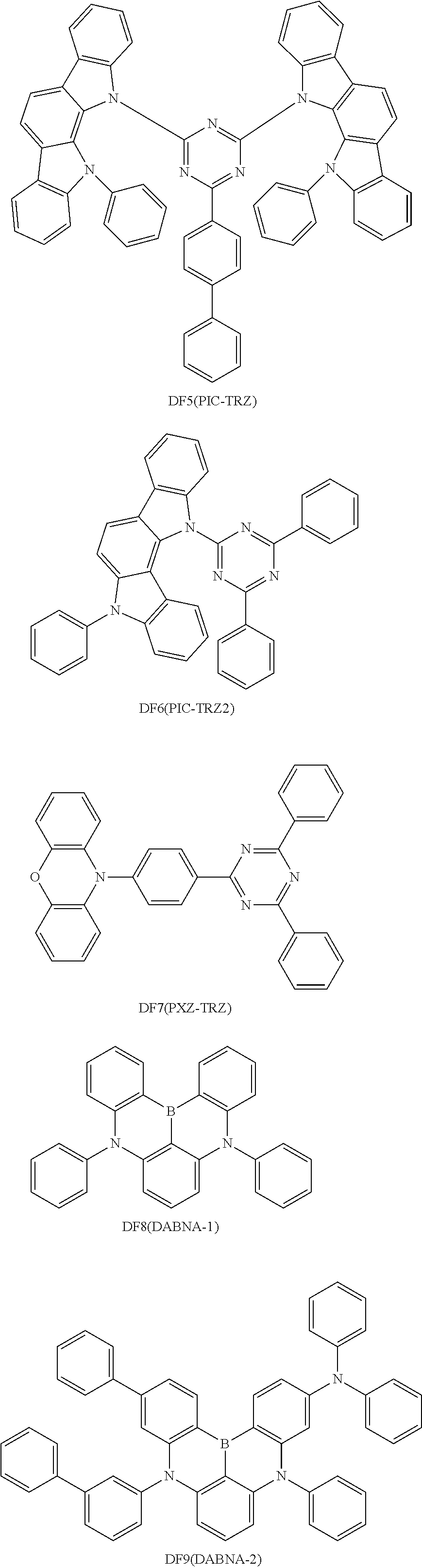 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccc(N4c5ccccc5Oc5ccccc54)cc3)n2)cc1 ; c1ccc(N2c3ccccc3B3c4ccccc4N(c4ccccc4)c4cccc2c43)cc1 ; c1ccc(-c2cccc(N3c4cc(-c5ccccc5)ccc4B4c5ccc(N(c6ccccc6)c6ccccc6)cc5N(c5ccccc5)c5cccc3c54)c2)cc1]

### Quantum Dot

The emission layer may include a quantum dot.

In the present specification, a quantum dot refers to a crystal of a semiconductor compound, and may include any material capable of emitting light of various emission wavelengths according to the size of the crystal.

A diameter of the quantum dot may be, for example, in a range of about 1 nm to about 10 nm.

The quantum dot may be synthesized by a wet chemical process, a metal organic chemical vapor deposition process, a molecular beam epitaxy process, or any process similar thereto.

According to the wet chemical process, a precursor material is mixed with an organic solvent to grow a quantum dot particle crystal. When the crystal grows, the organic solvent naturally acts as a dispersant coordinated on the surface of the quantum dot crystal and controls the growth of the crystal so that the growth of quantum dot particles can be controlled through a process which is more easily performed than vapor deposition methods, such as metal organic chemical vapor deposition (MOCVD) or molecular beam epitaxy (MBE), and which requires low costs.

The quantum dot may include Groups II-VI semiconductor compounds, Groups III-V semiconductor compounds, Groups III-VI semiconductor compounds, Group I-III-VI semiconductor compounds, Groups IV-VI semiconductor compounds, a Group IV element or compound; or any combination thereof.

Examples of the Groups II-VI semiconductor compound include a binary compound, such as CdSe, CdTe, ZnS, ZnSe, ZnTe, ZnO, HgS, HgSe, HgTe, MgSe, or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, or MgZnS; a quaternary compound, such as CdZnSeS, CdZnSeTe, CdZnSTe, CdHgSeS, CdHgSeTe, CdHgSTe, HgZnSeS, HgZnSeTe, or HgZnSTe; or any combination thereof.

Examples of the Group III-V semiconductor compound include a binary compound, such as GaN, GaP, GaAs, GaSb, AlN, AlP, AlAs, AlSb, InN, InP, InAs, InSb, or the like; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, InPSb, GaAlNP, or the like; a quaternary compound, such as GaAlNAs, GaAlNSb, GaAlPAs, GaAlPSb, GaInNP, GaInNAs, GalnNSb, GaInPAs, GalnPSb, InAlNP, InAlNAs, InAlNSb, InAlPAs, InAlPSb, or the like; or any combination thereof. Meanwhile, the Groups III-V semiconductor compound may further include Group II elements. Examples of the Groups III-V further including Group II elements are InZnP, InGaZnP, InAlZnP, etc.

Examples of the Groups III-VI semiconductor compound include a binary compound, such as GaS, GaSe, Ga₂Se₃, GaTe, InS, InSe, In₂S₃, In₂Se₃, or InTe; a ternary compound, such as InGaS₃, or InGaSe₃; and any combination thereof.

Examples of the Group I-III-VI semiconductor compound are a ternary compound, such as AgInS, AgInS₂, CuInS, CuInS₂, CuGaO₂, AgGaO₂, or AgAlO₂; or any combination thereof.

Examples of the Group IV-VI semiconductor compound are a binary compound, such as SnS, SnSe, SnTe, PbS, PbSe, PbTe, or the like; a ternary compound, such as SnSeS, SnSeTe, SnSTe, PbSeS, PbSeTe, PbSTe, SnPbS, SnPbSe, SnPbTe, or the like; a quaternary compound, such as SnPbSSe, SnPbSeTe, SnPbSTe, or the like; or any combination thereof.

The Group IV element or compound may include a single element compound, such as Si or Ge; a binary compound, such as SiC or SiGe; or any combination thereof.

Each element included in a multi-element compound such as the binary compound, ternary compound and quaternary compound, may exist in a particle with a uniform concentration or non-uniform concentration.

Meanwhile, the quantum dot may have a single structure or a dual core-shell structure. In the case of the quantum dot having a single structure, the concentration of each element included in the corresponding quantum dot is uniform. In one or more embodiments, the material contained in the core and the material contained in the shell may be different from each other.

The shell of the quantum dot may act as a protective layer to prevent chemical degeneration of the core to maintain semiconductor characteristics and/or as a charging layer to impart electrophoretic characteristics to the quantum dot. The shell may be a single layer or a multi-layer. The interface between the core and the shell may have a concentration gradient that decreases toward the center of the element present in the shell.

Examples of the shell of the quantum dot may include an oxide of metal, metalloid, or non-metal, a semiconductor compound, or any combination thereof. Examples of the oxide of metal, metalloid, or non-metal are a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO; a ternary compound, such as MgAl₂O₄, CoFe₂O₄, NiFe₂O₄, or CoMn₂O₄; or any combination thereof. Examples of the semiconductor compound are, as described herein, Groups II-VI semiconductor compounds; Groups III-V semiconductor compounds; Groups III-VI semiconductor compounds; Group I-III-VI semiconductor compounds; Groups IV-VI semiconductor compounds; and any combination thereof. In addition, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of an emission wavelength spectrum of the quantum dot may be about 45 nm or less, for example, about 40 nm or less, for example, about 30 nm or less, and within these ranges, color purity or color gamut may be increased. In addition, since the light emitted through the quantum dot is emitted in all directions, the wide viewing angle can be improved.

In addition, the quantum dot may be a spherical particle, a pyramidal particle, a multi-arm particle, a cubic nanoparticle, a nanotube particle, a nanowire particle, a nanofiber particle, or a nanoplate particle.

Since the energy band gap can be adjusted by controlling the size of the quantum dot, light having various wavelength bands can be obtained from the quantum dot emission layer. Therefore, by using quantum dots of different sizes, a light-emitting display that emits light of various wavelengths may be implemented. In one embodiment, the size of the quantum dot may be selected to emit red, green and/or blue light. In addition, the size of the quantum dot may be configured to emit white light by combining light of various colors.

### Electron Transport Region in Interlayer 130

The electron transport region may have: i) a single-layered structure consisting of a single layer consisting of a single material, ii) a single-layered structure consisting of a single layer consisting of a plurality of different materials, or iii) a multi-layered structure including a plurality of layers including different materials.

The electron transport region may include a buffer layer, a hole blocking layer, an electron control layer, an electron transport layer, an electron injection layer, or any combination thereof.

In one or more embodiments, the electron transport region may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, wherein, for each structure, constituting layers are sequentially stacked from an emission layer.

In one or more embodiments, the electron transport region (for example, the buffer layer, the hole blocking layer, the electron control layer, or the electron transport layer in the electron transport region) may include a metal-free compound including at least one π electron-deficient nitrogen-containing C₁-C₆₀ cyclic group.

In one or more embodiments, the electron transport region may include a compound represented by Formula 601 below:

[Ar₆₀₁]ₓₑ₁₁-[(L₆₀₁)ₓₑ₁-R₆₀₁]ₓₑ₂₁ Formula 601

wherein, in Formula 601,

Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xe11 may be 1, 2, or 3,

xe1 may be 0, 1, 2, 3, 4, or 5,

R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),

Q₆₀₁ to Q₆₀₃ are the same as described in connection with Q₁,

xe21 may be 1, 2, 3, 4, or 5,

at least one of Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, when xe11 in Formula 601 is 2 or more, two or more of Ar₆₀₁(s) may be linked via a single bond.

In one or more embodiments, Ar₆₀₁ in Formula 601 may be a substituted or unsubstituted anthracene group.

In one or more embodiments, the electron transport region may include a compound represented by Formula 601-1:

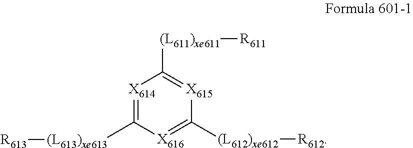

In Formula 601-1,

X₆₁₄ may be N or C(R₆₁₄), X₆₁₅ may be N or C(R₆₁₅), X₆₁₆ may be N or C(R₆₁₆), at least one of X₆₁₄ to X₆₁₆ may be N,

L₆₁₁ to L₆₁₃ are the same as described in connection with L₆₀₁,

xe611 to xe613 are the same as described in connection with xe1,

R₆₁₁ to R₆₁₃ are the same as described in connection with R₆₀₁,

R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group substituted or unsubstituted at least one R₁₀ₐ.

For example, xe1 and xe611 to xe613 in Formulae 601 and 601-1 may each independently be 0, 1, or 2.

The electron transport region may include one of Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, or any combination thereof:

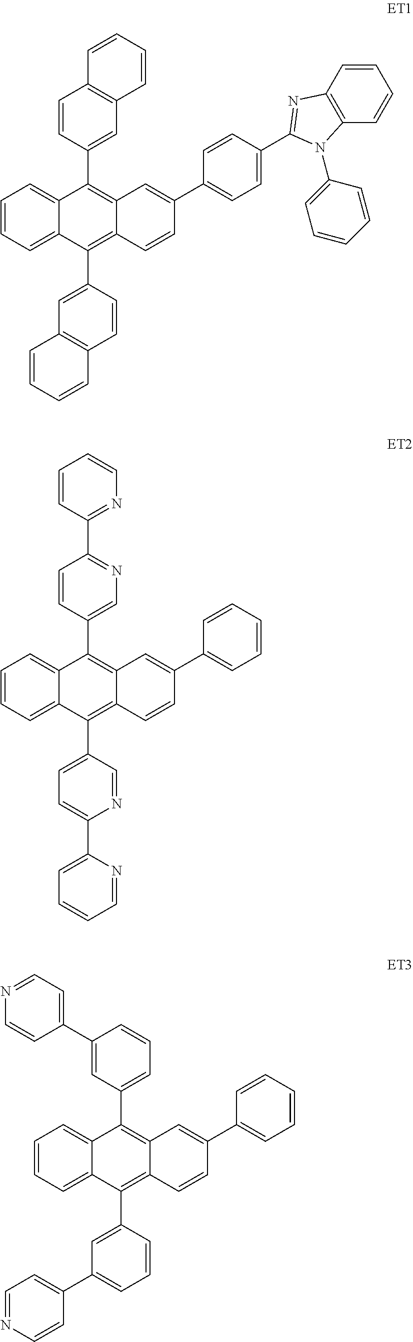 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

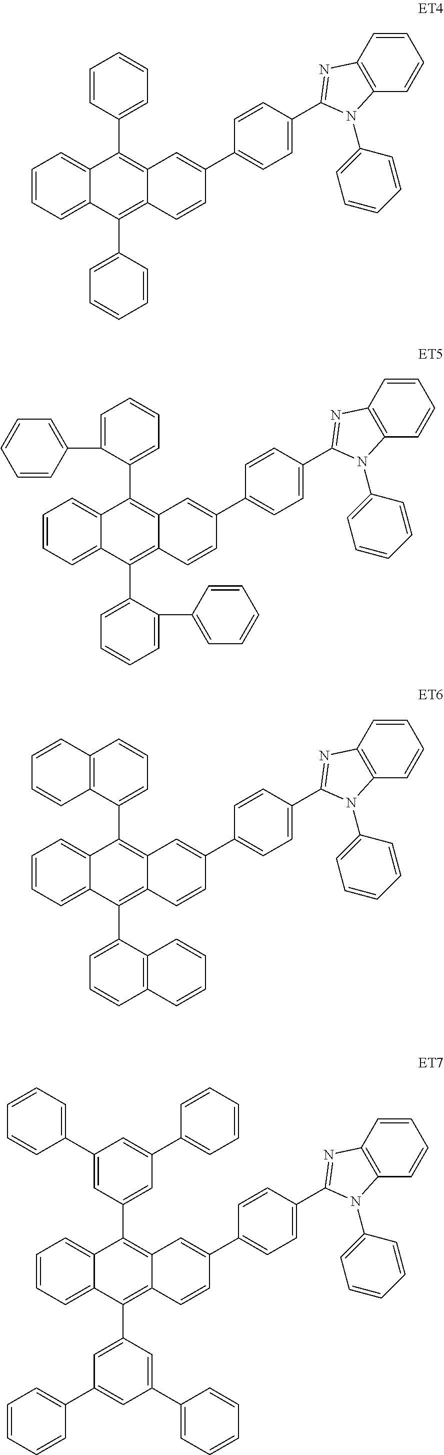 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

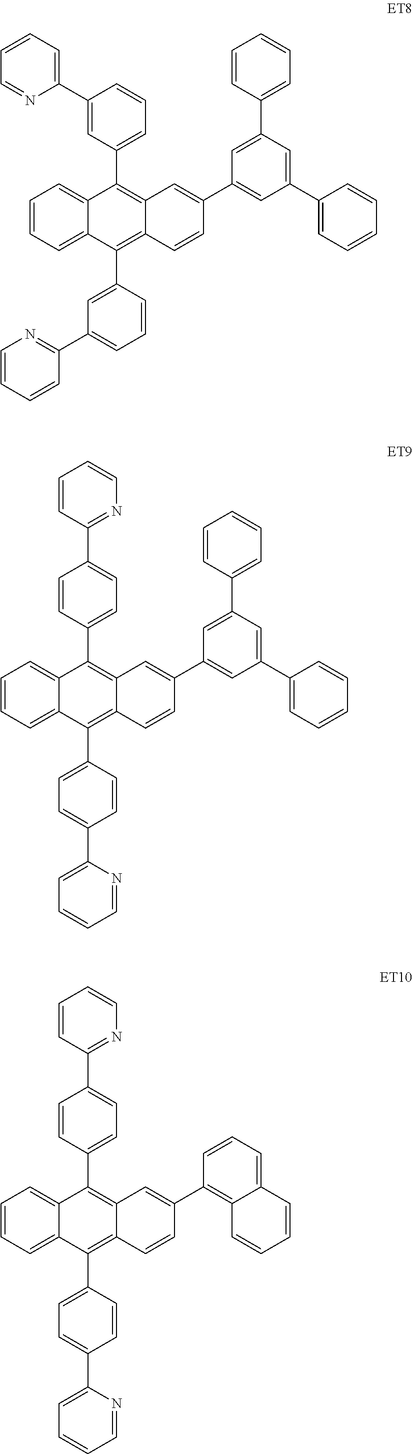 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

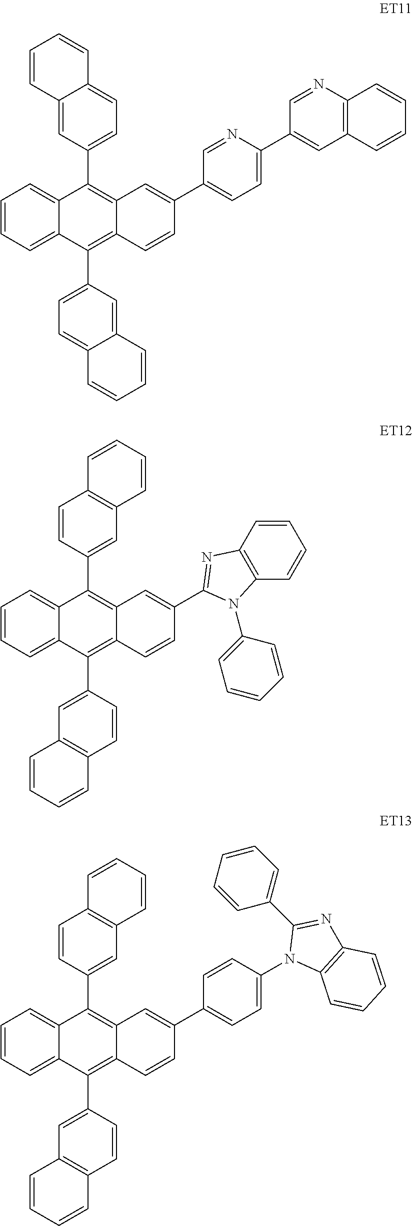

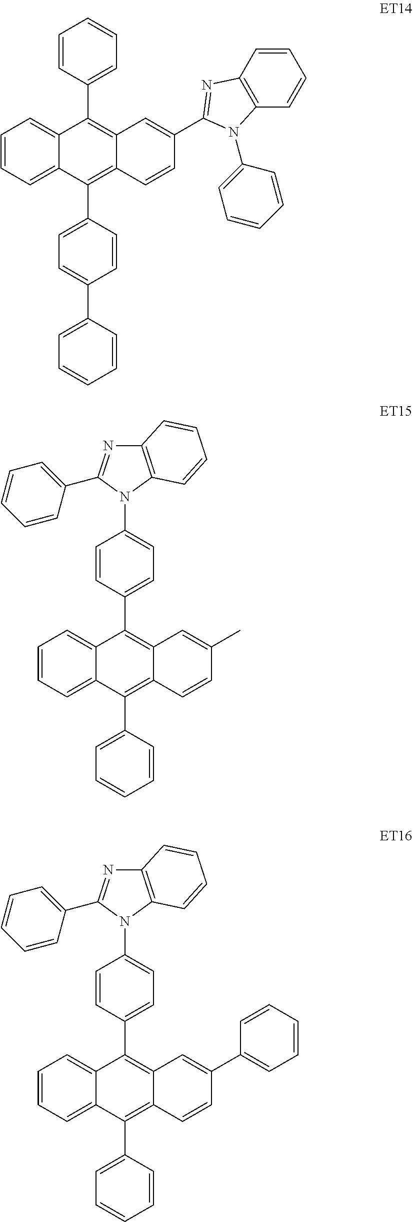 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

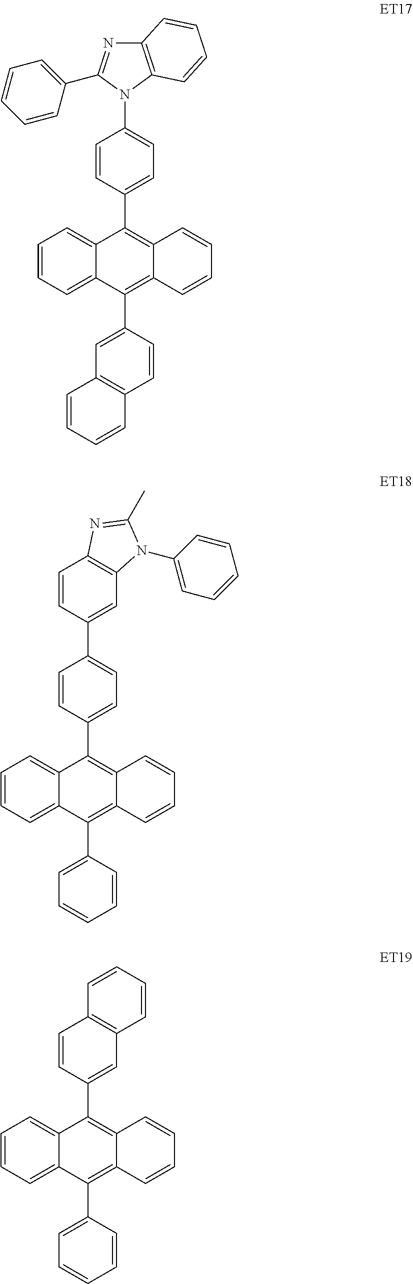

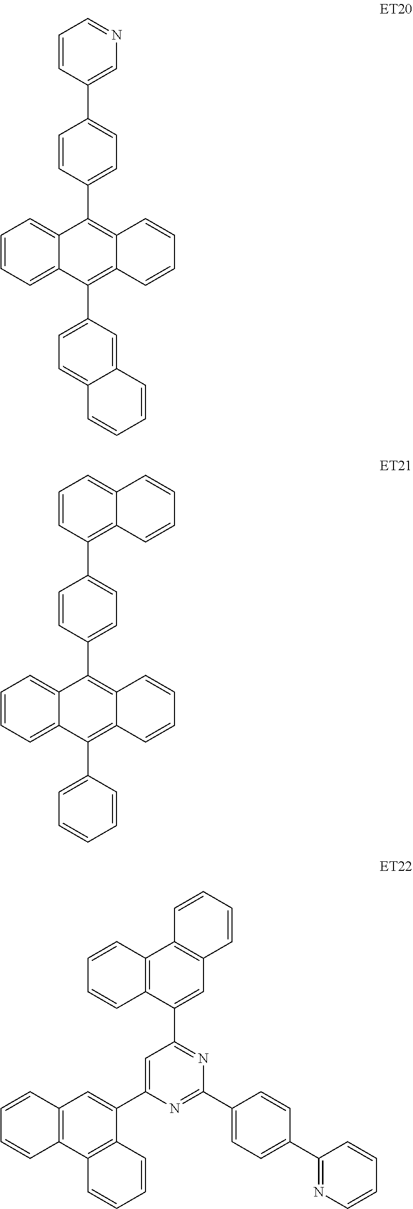

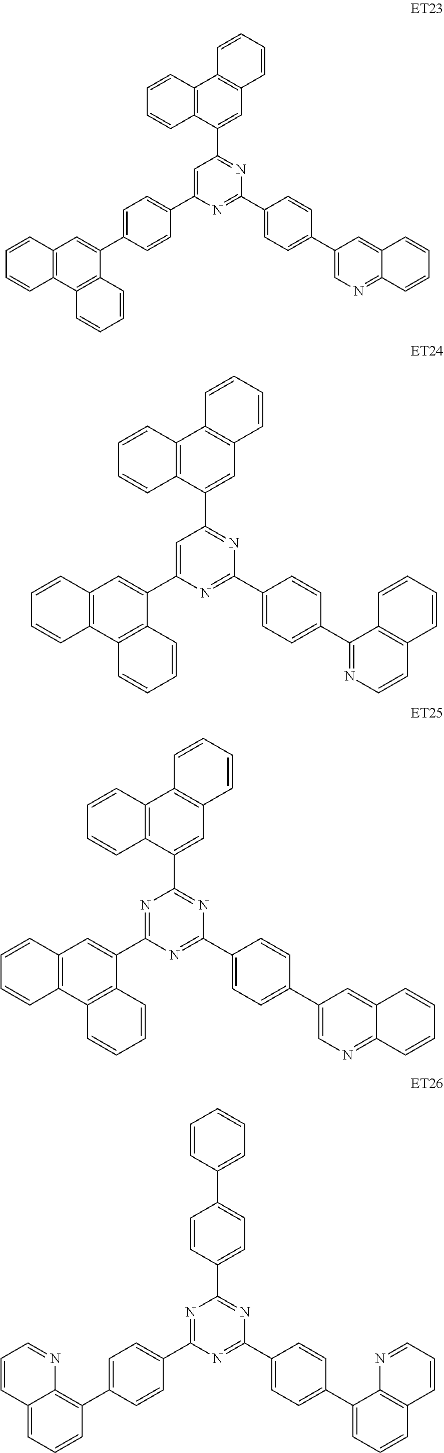 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1]

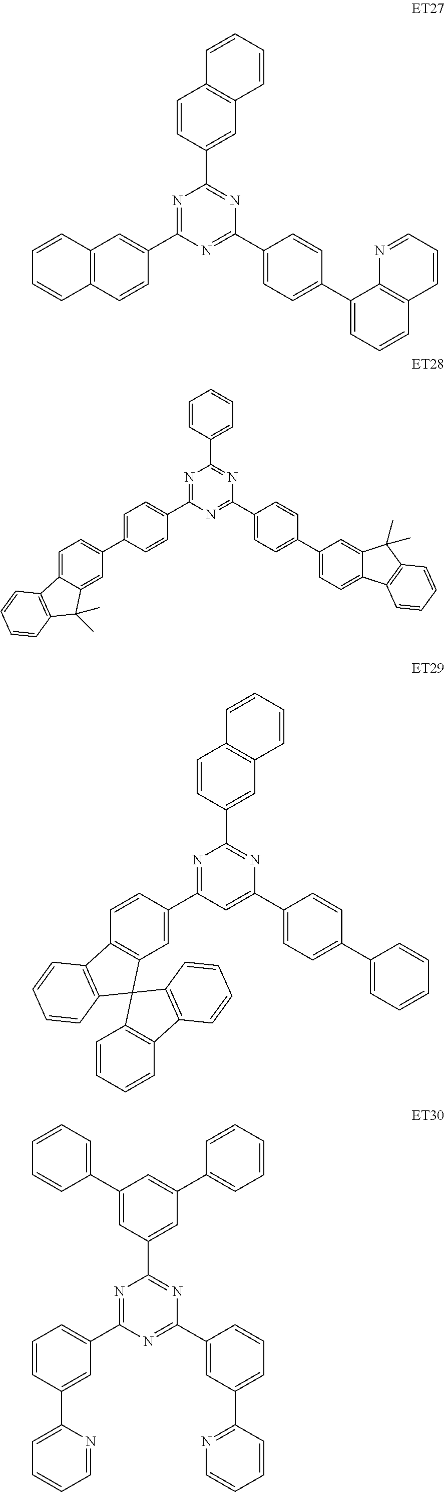 [SMILES: c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

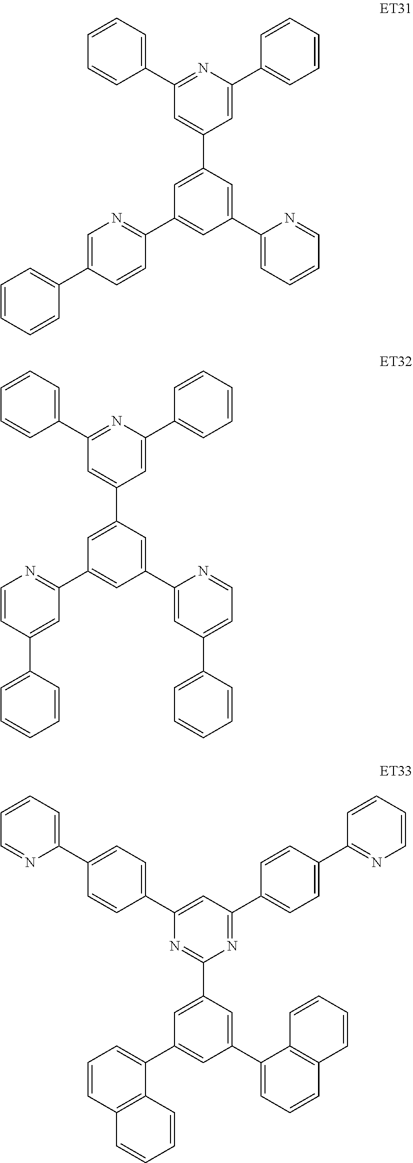

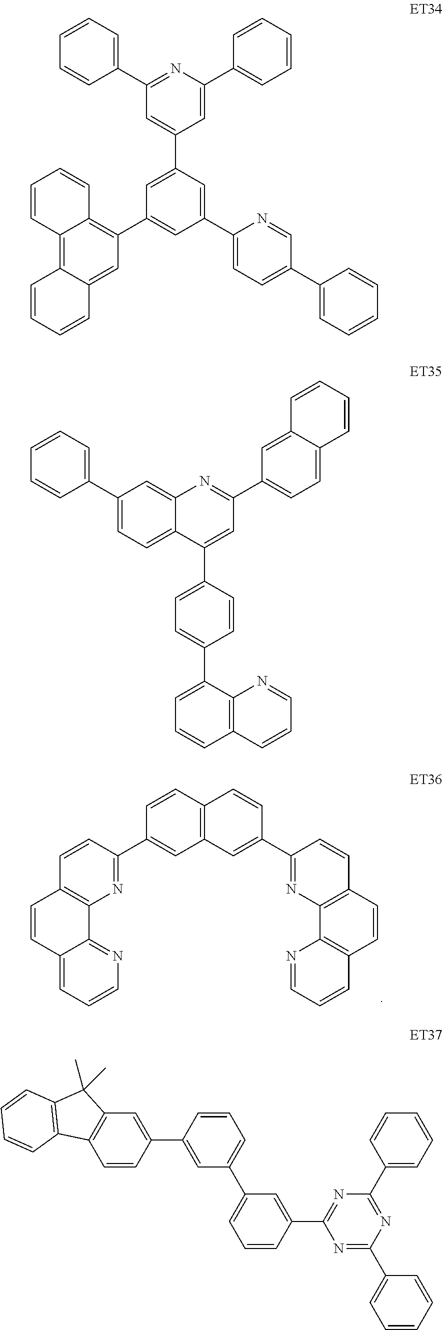 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12 ; CC1(C)c2ccccc2-c2ccc(-c3cccc(-c4cccc(-c5nc(-c6ccccc6)nc(-c6ccccc6)n5)c4)c3)cc21]

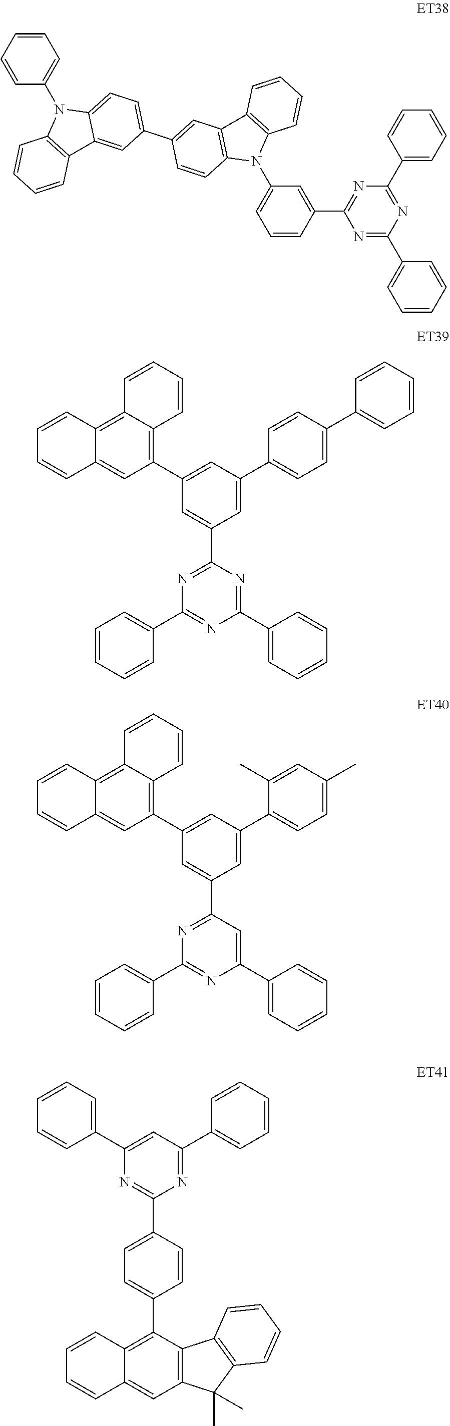

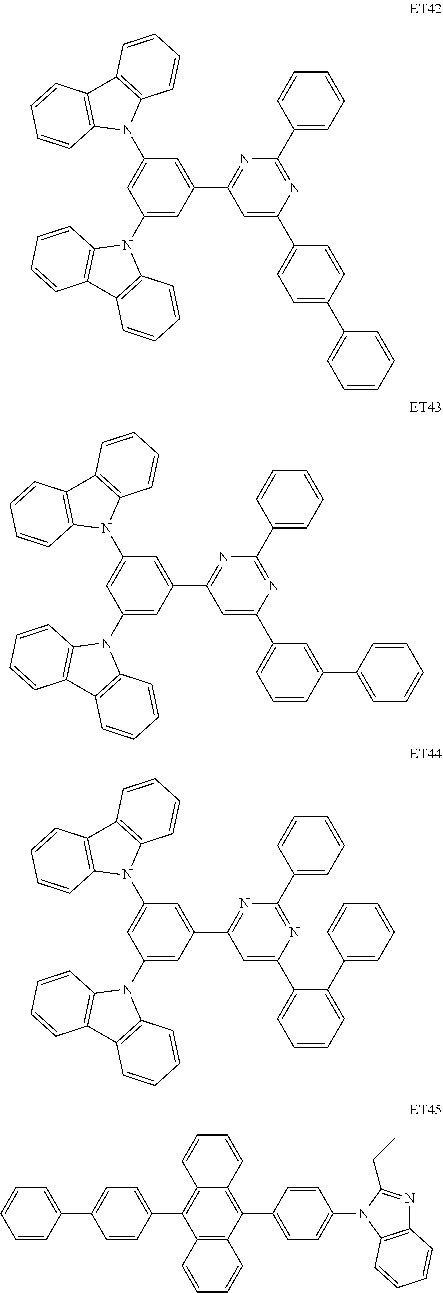

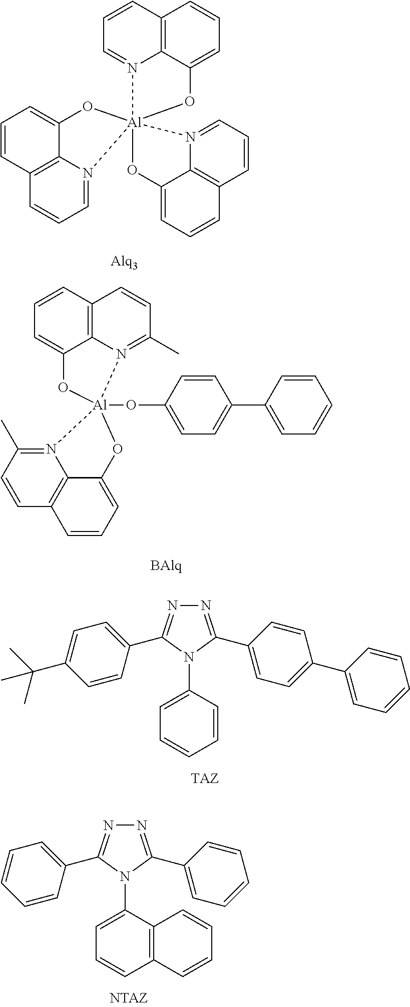

The thickness of the electron transport region may be from about 160 Å to about 5,000 Å, for example, from about 100 Å to about 4,000 Å. When the electron transport region includes a buffer layer, a hole blocking layer, an electron control layer, an electron transport layer, or any combination thereof, the thickness of the buffer layer, the hole blocking layer, or the electron control layer may each independently be from about 20 Å to about 1000 Å, for example, about 30 Å to about 300 Å, and the thickness of the electron transport layer may be from about 100 Å to about 1000 Å, for example, about 150 Å to about 500 Å. When the thicknesses of the buffer layer, hole blocking layer, electron control layer, electron transport layer and/or electron transport layer are within these ranges, satisfactory electron transporting characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, alkaline earth metal complex, or any combination thereof. The metal ion of an alkali metal complex may be a Li ion, a Na ion, a K ion, a Rb ion, or a Cs ion, and the metal ion of alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

For example, the metal-containing material may include a Li complex. The Li complex may include, for example, Compound ET-D1 (LiQ) or ET-D2:

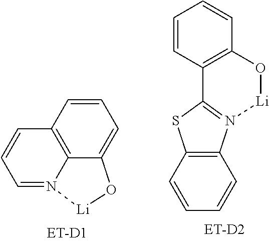

The electron transport region may include an electron injection layer that facilitates the injection of electrons from the second electrode 150. The electron injection layer may directly contact the second electrode 150.

The electron injection layer may have: i) a single-layered structure consisting of a single layer consisting of a single material, ii) a single-layered structure consisting of a single layer consisting of a plurality of different materials, or iii) a multi-layered structure including a plurality of layers including different materials.

The electron injection layer may include an alkali metal, alkaline earth metal, a rare earth metal, an alkali metal-containing compound, alkaline earth metal-containing compound, a rare earth metal-containing compound, an alkali metal complex, alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (for example, fluorides, chlorides, bromides, or iodides), or tellurides of the alkali metal, the alkaline earth metal, and the rare earth metal, or any combination thereof.

The alkali metal-containing compound may include alkali metal oxides, such as Li₂O, Cs₂O, or K₂O, alkali metal halides, such as LiF, NaF, CsF, KF, LiI, NaI, CsI, or KI, or any combination thereof. The alkaline earth metal-containing compound may include an alkaline earth metal compound, such as BaO, SrO, CaO, BaₓSr₁₋ₓO (x is a real number satisfying the condition of 0<x<1), BaₓCa₁₋ₓO (x is a real number satisfying the condition of 0<x<1), or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal telluride. Examples of the lanthanide metal telluride are LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and Lu₂Te₃.

The alkali metal complex, the alkaline earth-metal complex, and the rare earth metal complex may include i) one of ions of the alkali metal, the alkaline earth metal, and the rare earth metal and ii), as a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

The electron injection layer may consist of an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (for example, a compound represented by Formula 601).

In one or more embodiments, the electron injection layer may consist of i) an alkali metal-containing compound (for example, an alkali metal halide), ii) a) an alkali metal-containing compound (for example, an alkali metal halide); and b) an alkali metal, an alkaline earth metal, a rare earth metal, or any combination thereof. In one or more embodiments, the electron injection layer may be a KI:Yb co-deposited layer, an RbI:Yb co-deposited layer, or the like.

When the electron injection layer further includes an organic material, alkali metal, alkaline earth metal, rare earth metal, an alkali metal-containing compound, an alkaline earth metal-containing compound, a rare earth metal-containing compound, alkali metal complex, alkaline earth-metal complex, rare earth metal complex, or any combination thereof may be homogeneously or non-homogeneously dispersed in a matrix including the organic material.

A thickness of the electron injection layer may be in a range of about 1 Å to about 100 Å, and, for example, about 3 Å to about 90 Å. When the thickness of the electron injection layer is within the range described above, the electron injection layer may have satisfactory electron injection characteristics without a substantial increase in driving voltage.

### Second Electrode 150

The second electrode 150 may be located on the interlayer 130 having such a structure. The second electrode 150 may be a cathode, which is an electron injection electrode, and as the material for the second electrode 150, a metal, an alloy, an electrically conductive compound, or any combination thereof, each having a low work function, may be used.

In one or more embodiments, the second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or a combination thereof. The second electrode 150 may be a transmissive electrode, a semi-transmissive electrode, or a reflective electrode.

The second electrode 150 may have a single-layered structure or a multi-layered structure including two or more layers.

### Capping Layer

A first capping layer may be located outside the first electrode 110, and/or a second capping layer may be located outside the second electrode 150. In detail, the light-emitting device 10 may have a structure in which the first capping layer, the first electrode 110, the interlayer 130, and the second electrode 150 are sequentially stacked in this stated order, a structure in which the first electrode 110, the interlayer 130, the second electrode 150, and the second capping layer are sequentially stacked in this stated order, or a structure in which the first capping layer, the first electrode 110, the interlayer 130, the second electrode 150, and the second capping layer are sequentially stacked in this stated order.

Light generated in an emission layer of the interlayer 130 of the light-emitting device 10 may be extracted toward the outside through the first electrode 110, which is a semi-transmissive electrode or a transmissive electrode, and the first capping layer or light generated in an emission layer of the interlayer 130 of the light-emitting device 10 may be extracted toward the outside through the second electrode 150, which is a semi-transmissive electrode or a transmissive electrode, and the second capping layer.

The first capping layer and the second capping layer may increase external emission efficiency according to the principle of constructive interference. Accordingly, the light extraction efficiency of the light-emitting device 10 is increased, so that the emission efficiency of the light-emitting device 10 may be improved.

Each of the first capping layer and second capping layer may include a material having a refractive index (at 589 nm) of about 1.6 or more.

The first capping layer and the second capping layer may each independently be an organic capping layer including an organic material, an inorganic capping layer including an inorganic material, or an organic-inorganic composite capping layer including an organic material and an inorganic material.

At least one of the first capping layer, the second capping layer, or any combination thereof may each independently include carbocyclic compounds, heterocyclic compounds, amine group-containing compounds, porphyrin derivatives, phthalocyanine derivatives, naphthalocyanine derivatives, alkali metal complexes, alkaline earth metal complexes, or any combination thereof. The carbocyclic compound, the heterocyclic compound, and the amine group-containing compound may be optionally substituted with a substituent containing O, N, S, Se, Si, F, Cl, Br, I, or any combination thereof. In one or more embodiments, at least one of the first capping layer and the second capping layer may each independently include an amine group-containing compound.

In one or more embodiments, at least one of the first capping layer, the second capping layer, or any combination thereof may each independently include a compound represented by Formula 201, a compound represented by Formula 202, or any combination thereof.

In one or more embodiments, at least one of the first capping layer, the second capping layer, or any combination thereof may each independently include one of Compounds HT28 to HT33, one of Compounds CP1 to CP6, β-NPB, or any combination thereof:

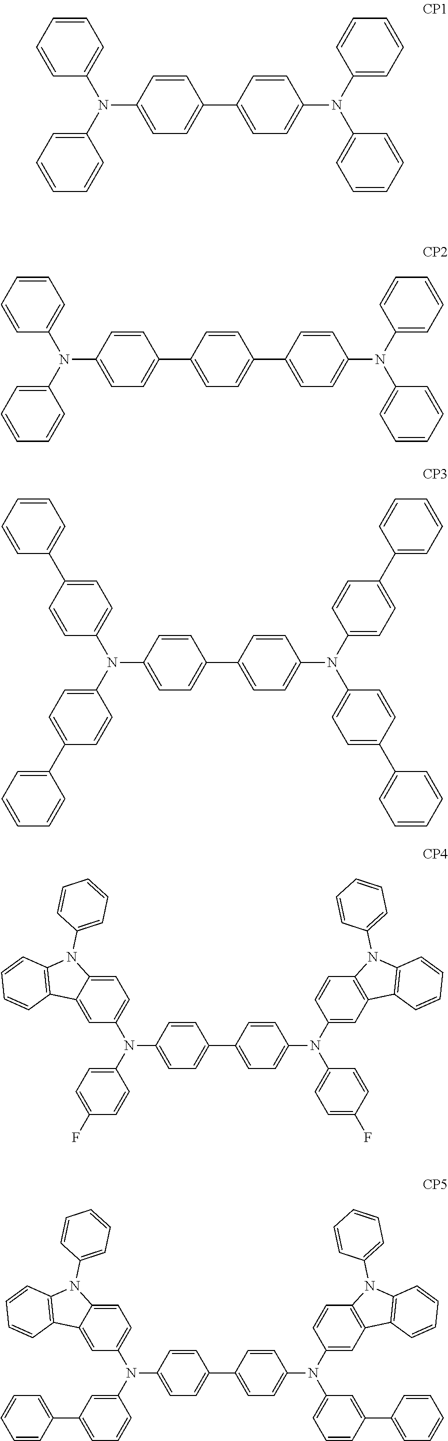 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1]

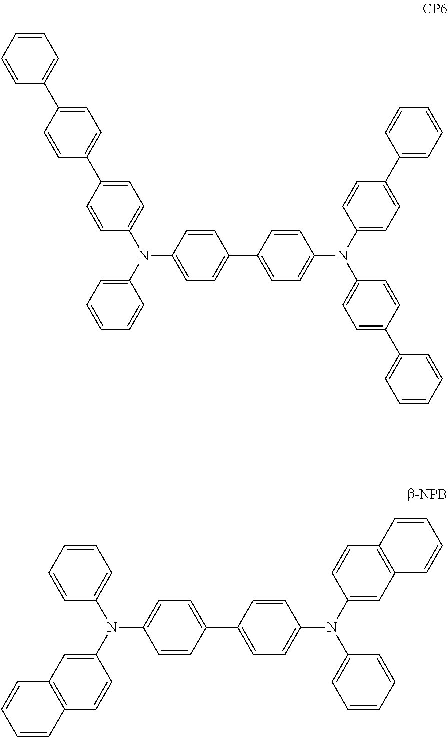 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Electronic Apparatus

The light-emitting device may be included in various electronic apparatuses. In one or more embodiments, the electronic apparatus including the light-emitting device may be a light-emitting apparatus, an authentication apparatus, or the like.

The electronic apparatus (for example, light-emitting apparatus) may further include, in addition to the light-emitting device, i) a color filter, ii) a color conversion layer, or iii) a color filter and a color conversion layer. The color filter and/or the color conversion layer may be located in at least one traveling direction of light emitted from the light-emitting device. In one or more embodiments, the light emitted from the light-emitting device may be blue light or white light. The light-emitting device may be the same as described above. In one or more embodiments, the color conversion layer may include quantum dots. The quantum dot may be, for example, a quantum dot as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be located among the subpixel areas to define each of the subpixel areas.

The color filter may further include a plurality of color filter areas and light-blocking patterns located among the color filter areas, and the color conversion layer may include a plurality of color conversion areas and light-blocking patterns located among the color conversion areas.

The color filter areas (or the color conversion areas) may include a first area emitting first color light, a second area emitting second color light, and/or a third area emitting third color light, and the first color light, the second color light, and/or the third color light may have different maximum emission wavelengths from one another. In one or more embodiments, the first color light may be red light, the second color light may be green light, and the third color light may be blue light. In one or more embodiments, the color filter areas (or the color conversion areas) may include quantum dots. In detail, the first area may include a red quantum dot, the second area may include a green quantum dot, and the third area may not include a quantum dot. The quantum dot is the same as described in the present specification. The first area, the second area, and/or the third area may each include a scatter.

In one or more embodiments, the light-emitting device may emit first light, the first area may absorb the first light to emit first first-color light, the second area may absorb the first light to emit second first-color light, and the third area may absorb the first light to emit third first-color light. In this regard, the first first-color light, the second first-color light, and the third first-color light may have different maximum emission wavelengths. In detail, the first light may be blue light, the first first-color light may be red light, the second first-color light may be green light, and the third first-color light may be blue light.

The electronic apparatus may further include a thin-film transistor in addition to the light-emitting device 1 as described above. The thin-film transistor may include a source electrode, a drain electrode, and an activation layer, wherein any one of the source electrode and the drain electrode may be electrically connected to any one of the first electrode and the second electrode of the light-emitting device.

The thin-film transistor may further include a gate electrode, a gate insulating film, etc.

The activation layer may include crystalline silicon, amorphous silicon, organic semiconductor, oxide semiconductor, or the like.

The electronic apparatus may further include a sealing portion for sealing the light-emitting device. The sealing portion and/or the color conversion layer may be placed between the color filter and the light-emitting device. The sealing portion allows light from the light-emitting device to be extracted to the outside, while simultaneously preventing ambient air and moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin film encapsulation layer, the electronic apparatus may be flexible.

Various functional layers may be additionally located on the sealing portion, in addition to the color filter and/or the color conversion layer, according to the use of the electronic apparatus. The functional layers may include a touch screen layer, a polarizing layer, and the like. The touch screen layer may be a pressure-sensitive touch screen layer, a capacitive touch screen layer, or an infrared touch screen layer. The authentication apparatus may be, for example, a biometric authentication apparatus that authenticates an individual by using biometric information of a living body (for example, fingertips, pupils, etc.).

The authentication apparatus may further include, in addition to the light-emitting device, a biometric information collector.

The electronic apparatus may be applied to various displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, various measuring instruments, meters (for example, meters for a vehicle, an aircraft, and a vessel), projectors, and the like.

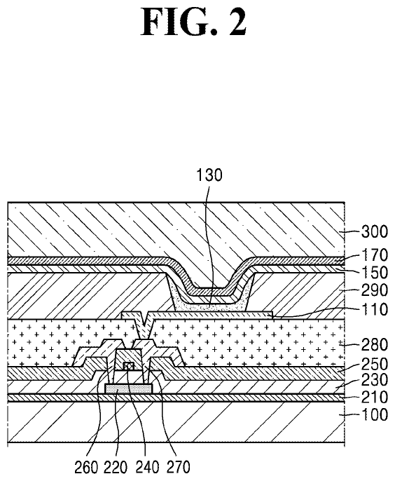

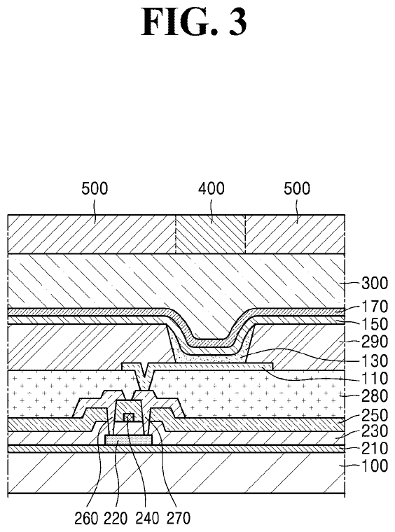

### Description of FIGS. 2 and 3

FIG. 2 is a cross-sectional view showing a light-emitting apparatus according to an exemplary embodiment of the present disclosure.

The light-emitting apparatus of FIG. 2 includes a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be formed on the substrate 100. The buffer layer 210 may prevent penetration of impurities through the substrate 100 and may provide a flat surface on the substrate 100.

A TFT may be located on the buffer layer 210. The TFT may include an activation layer 220, a gate electrode 240, a source electrode 260, and a drain electrode 270.

The activation layer 220 may include an inorganic semiconductor such as silicon or polysilicon, an organic semiconductor, or an oxide semiconductor, and may include a source region, a drain region and a channel region.

A gate insulating film 230 for insulating the activation layer 220 from the gate electrode 240 may be located on the activation layer 220, and the gate electrode 240 may be located on the gate insulating film 230.

An interlayer insulating film 250 is located on the gate electrode 240. The interlayer insulating film 250 may be placed between the gate electrode 240 and the source electrode 260 to insulate the gate electrode 240 from the source electrode 260 and between the gate electrode 240 and the drain electrode 270 to insulate the gate electrode 240 from the drain electrode 270.

The source electrode 260 and the drain electrode 270 may be located on the interlayer insulating film 250. The interlayer insulating film 250 and the gate insulating film 230 may be formed to expose the source region and the drain region of the activation layer 220, and the source electrode 260 and the drain electrode 270 may be in contact with the exposed portions of the source region and the drain region of the activation layer 220.

The TFT is electrically connected to a light-emitting device to drive the light-emitting device, and is covered by a passivation layer 280. The passivation layer 280 may include an inorganic insulating film, an organic insulating film, or a combination thereof. A light-emitting device is provided on the passivation layer 280. The light-emitting device may include a first electrode 110, an interlayer 130, and a second electrode 150.

The first electrode 110 may be formed on the passivation layer 280. The passivation layer 280 does not completely cover the drain electrode 270 and exposes a portion of the drain electrode 270, and the first electrode 110 is connected to the exposed portion of the drain electrode 270.

A pixel defining layer 290 containing an insulating material may be located on the first electrode 110. The pixel defining layer 290 exposes a region of the first electrode 110, and an interlayer 130 may be formed in the exposed region of the first electrode 110. The pixel defining layer 290 may be a polyimide or polyacrylic organic film. Although not shown in FIG. 2, at least some layers of the interlayer 130 may extend beyond the upper portion of the pixel defining layer 290 to be located in the form of a common layer.

The second electrode 150 may be located on the interlayer 130, and a capping layer 170 may be additionally formed on the second electrode 150. The capping layer 170 may be formed to cover the second electrode 150.

The encapsulation portion 300 may be located on the capping layer 170. The encapsulation portion 300 may be located on a light-emitting device to protect the light-emitting device from moisture or oxygen. The encapsulation portion 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, or the like), an epoxy-based resin (for example, aliphatic glycidyl ether (AGE), or the like), or a combination thereof; or a combination of the inorganic film and the organic film.

FIG. 3 shows a cross-sectional view showing a light-emitting apparatus according to an exemplary embodiment of the present disclosure.

The light-emitting apparatus of FIG. 3 is the same as the light-emitting apparatus of FIG. 2, except that a light-blocking pattern 500 and a functional region 400 are additionally located on the encapsulation portion 300. The functional region 400 may be a combination of i) a color filter area, ii) a color conversion area, or iii) a combination of the color filter area and the color conversion area. In one or more embodiments, the light-emitting device included in the light-emitting apparatus of FIG. 3 may be a tandem light-emitting device.

### Manufacture Method

Respective layers included in the hole transport region, the emission layer, and respective layers included in the electron transport region may be formed in a certain region by using one or more suitable methods selected from vacuum deposition, spin coating, casting, Langmuir-Blodgett (LB) deposition, ink-jet printing, laser-printing, and laser-induced thermal imaging.

When layers constituting the hole transport region, an emission layer, and layers constituting the electron transport region are formed by vacuum deposition, the deposition may be performed at a deposition temperature of about 100° C. to about 500° C., a vacuum pressure of about 10⁻⁸ torr to about 10⁻³ torr, and a deposition speed of about 0.01 Å/sec to about 100 Å/sec, depending on a material to be included in a layer to be formed and the structure of a layer to be formed.

### Definition of Terms

The term “C₃-C₆₀ carbocyclic group” as used herein refers to a cyclic group consisting of carbon only as a ring-forming atom and having three to sixty carbon atoms, and the term “C₁-C₆₀ heterocyclic group” as used herein refers to a cyclic group that has one to sixty carbon atoms and further has, in addition to carbon, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed with each other. For example, the C₁-C₆₀ heterocyclic group has 3 to 61 ring-forming atoms.

The “cyclic group” as used herein may include the C₃-C₆₀ carbocyclic group, and the C₁-C₆₀ heterocyclic group.

The term “π electron-rich C₃-C₆₀ cyclic group” as used herein refers to a cyclic group that has three to sixty carbon atoms and does not include *—N═*′ as a ring-forming moiety, and the term “π electron-deficient nitrogen-containing C₁-C₆₀ cyclic group” as used herein refers to a heterocyclic group that has one to sixty carbon atoms and includes *—N═*′ as a ring-forming moiety.

For example,

the C₃-C₆₀ carbocyclic group may be i) group T1 or ii) a condensed cyclic group in which two or more groups T1 are condensed with each other (for example, a cyclopentadiene group, an adamantane group, a norbornane group, a benzene group, a pentalene group, a naphthalene group, an azulene group, an indacene group, acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),

the C₁-C₆₀ heterocyclic group may be i) group T2, ii) a condensed cyclic group in which two or more groups T2 are condensed with each other, or iii) a condensed cyclic group in which at least one group T2 and at least one group T1 are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

the π electron-rich C₃-C₆₀ cyclic group may be i) group T1, ii) a condensed cyclic group in which two or more groups T1 are condensed with each other, iii) group T3, iv) a condensed cyclic group in which two or more groups T3 are condensed with each other, or v) a condensed cyclic group in which at least one group T3 and at least one group T1 are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.),

the π electron-deficient nitrogen-containing C₁-C₆₀ cyclic group may be i) group T4, ii) a condensed cyclic group in which two or more of group T4 are condensed with each other, iii) a condensed cyclic group in which at least one of group T4 and at least one of group T1 are condensed with each other, iv) a condensed cyclic group in which at least one of group T4 and at least one of group T3 are condensed with each other, or v) a condensed cyclic group in which at least one of group T4, at least one of group T1, and at least one of group T3 are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

group T1 may include a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,

group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, tetrahydropyrazine group, a dihydropyrazine group, a tetrahydropyridazine group, or a dihydropyridazine group,

group T3 may include a furan group, a thiophene group, a 1H-pyrrole group, a silole group, or a borole group, and

group T4 may include a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group, the C₃-C₆₀ carbocyclic group, the C₁-C₆₀ heterocyclic group, the π electron-rich C₃-C₆₀ cyclic group, or the π electron-deficient nitrogen-containing C₁-C₆₀ cyclic group” as used herein refer to a group condensed to any cyclic group or a polyvalent group (for example, a divalent group, a trivalent group, a tetravalent group, etc.), depending on the structure of a formula in connection with which the terms are used. In one or more embodiments, “a benzene group” may be a benzo group, a phenyl group, a phenylene group, or the like, which may be easily understood by one of ordinary skill in the art according to the structure of a formula including the “benzene group.”

Examples of the monovalent C₃-C₆₀ carbocyclic group and the monovalent C₁-C₆₀ heterocyclic group are a C₃-C₁₀ cycloalkyl group, a C₁-C₁₀ heterocycloalkyl group, a C₃-C₁₀ cycloalkenyl group, a C₁-C₁₀heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group, and examples of the divalent C₃-C₆₀ carbocyclic group and the monovalent C₁-C₆₀ heterocyclic group are Examples of the, C₃-C₁₀ cycloalkylene group, a C₁-C₁₀heterocycloalkylene group, a C₃-C₁₀ cycloalkenylene group, a C₁-C₁₀heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group” as used herein refers to a linear or branched aliphatic hydrocarbon monovalent group that has one to sixty carbon atoms, and examples thereof are a methyl group, an ethyl group, an n-propyl group, an isopropyl group, an n-butyl group, a sec-butyl group, an isobutyl group, a tert-butyl group, an n-pentyl group, a tert-pentyl group, neopentyl group, an isopentyl group, a sec-pentyl group, 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as used herein refers to a divalent group having the same structure as the C₁-C₆₀ alkyl group.

The term “C₂-C₆₀ alkenyl group” as used herein refers to a monovalent hydrocarbon group having at least one carbon-carbon double bond in the middle or at the terminus of the C₂-C₆₀ alkyl group, and examples thereof are an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a monovalent hydrocarbon group having at least one carbon-carbon triple bond in the middle or at the terminus of the C₂-C₆₀ alkyl group, and examples thereof include an ethynyl group, and a propynyl group. The term “C₁-C₆₀ alkynylene group” as used herein refers to a divalent group having the same structure as the C₁-C₆₀ alkynyl group.

The term “C₁-C₆₀ alkoxy group” as used herein refers to a monovalent group represented by —OA₁₀₁ (wherein A₁₀₁ is the C₁-C₆₀ alkyl group), and examples thereof include a methoxy group, an ethoxy group, and an isopropyloxy group.

The term “C₃-C₁₀ cycloalkyl group” as used herein refers to a monovalent saturated hydrocarbon cyclic group having 3 to 10 carbon atoms, and examples thereof are a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, and a bicyclo[2.2.2]octyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent cyclic group that further includes, in addition to a carbon atom, at least one heteroatom as a ring-forming atom and has 1 to 10 carbon atoms, and examples thereof are a 1,2,3,4-oxatriazolidinyl group, a tetrahydrofuranyl group, and a tetrahydrothiophenyl group. The term “C₁-C₁₀ heterocycloalkylene group” as used herein refers to a divalent group having the same structure as the C₁-C₁₀ heterocycloalkyl group.

The term C₃-C₁₀ cycloalkenyl group used herein refers to a monovalent cyclic group that has three to ten carbon atoms and at least one carbon-carbon double bond in the ring thereof and no aromaticity, and examples thereof are a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent cyclic group that has, in addition to a carbon atom, at least one heteroatom as a ring-forming atom, 1 to 10 carbon atoms, and at least one carbon-carbon double bond in the cyclic structure thereof. Examples of the C₁-C₁₀ heterocycloalkenyl group include a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system having six to sixty carbon atoms, and the term “C₆-C₆₀ arylene group” as used herein refers to a divalent group having a carbocyclic aromatic system having six to sixty carbon atoms. Examples of the C₆-C₆₀ aryl group are a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each include two or more rings, the rings may be condensed with each other.

The term “C₁-C₆₀ heteroaryl group” as used herein refers to a monovalent group having a heterocyclic aromatic system that has, in addition to a carbon atom, at least one heteroatom as a ring-forming atom, and 1 to 60 carbon atoms. The term “C₁-C₆₀ heteroarylene group” as used herein refers to a divalent group having a heterocyclic aromatic system that has, in addition to a carbon atom, at least one heteroatom as a ring-forming atom, and 1 to 60 carbon atoms. Examples of the C₁-C₆₀ heteroaryl group are a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the rings may be condensed with each other.

The term “monovalent non-aromatic condensed polycyclic group” as used herein refers to a monovalent group (for example, having 8 to 60 carbon atoms) having two or more rings condensed to each other, only carbon atoms as ring-forming atoms, and no aromaticity in its entire molecular structure. Examples of the monovalent non-aromatic condensed polycyclic group are an indenyl group, a fluorenyl group, a spiro-bifluorenyl group, a benzofluorenyl group, an indenophenanthrenyl group, and an indenon anthracenyl group. The term “divalent non-aromatic condensed polycyclic group” as used herein refers to a divalent group having the same structure as a monovalent non-aromatic condensed polycyclic group.

The term “monovalent non-aromatic condensed heteropolycyclic group” as used herein refers to a monovalent group (for example, having 1 to 60 carbon atoms) having two or more rings condensed to each other, at least one heteroatom other than carbon atoms, as a ring-forming atom, and no aromaticity in its entire molecular structure. Examples of the monovalent non-aromatic condensed heteropolycyclic group are a pyrrolyl group, a thiophenyl group, a furanyl group, an indolyl group, a benzoindolyl group, a naphthon indolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group. The term “divalent non-aromatic heterocondensed polycyclic group” as used herein refers to a divalent group having the same structure as a monovalent non-aromatic heterocondensed polycyclic group.

The term “C₆-C₆₀ aryloxy group” as used herein indicates —OA₁₀₂ (wherein A₁₀₂ is the C₆-C₆₀ aryl group), and the term “C₆-C₆₀ arylthio group” as used herein indicates —SA₁₀₃ (wherein A₁₀₃ is the C₆-C₆₀ aryl group).

The term “C₇-C₆₀ aryl alkyl group” used herein refers to -A₁₀₄-A₁₀₅ (where A₁₀₄ may be a C₁-C₅₄ alkylene group, and A₁₀₅ may be a C₆-C₅₉ aryl group), and the term C₂-C₆₀ heteroaryl alkyl group” used herein refers to -A₁₀₆A₁₀₇ (where A₁₀₆ may be a C₁-C₅₉ alkylene group, and A₁₀₇ may be a C₁-C₅₉ heteroaryl group).

R₁₀ₐ may be:

deuterium (-D), —F, —Cl, —Br, —I, a hydroxyl group, a cyano group, or a nitro group, a C₁-C₆₀ alkyl group, a C₂-C₆₀ alkenyl group, a C₂-C₆₀ alkynyl group, or a C₁-C₆₀ alkoxy group, each substituted or unsubstituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each substituted or unsubstituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

Q₁ to Q₃, Q₁₁ to Q₁₃, Q₂₁ to Q₂₃ and Q₃₁ to Q₃₃ used herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ aryl alkyl group; or a C₂-C₆₀ heteroaryl alkyl group.

The term “hetero atom” as used herein refers to any atom other than a carbon atom. Examples of the heteroatom include O, S, N, P, Si, B, Ge, Se, and any combination thereof.

The term “the third-row transition metal” used herein includes hafnium (Hf), tantalum (Ta), tungsten (W), rhenium (Re), osmium (Os), iridium (Ir), platinum (Pt), gold (Au), etc.

The term “Ph” as used herein refers to a phenyl group, the term “Me” as used herein refers to a methyl group, the term “Et” as used herein refers to an ethyl group, the term “tert-Bu” or “Bu^{t}” as used herein refers to a tert-butyl group, and the term “OMe” as used herein refers to a methoxy group.

The term “biphenyl group” as used herein refers to “a phenyl group substituted with a phenyl group.” In other words, the “biphenyl group” is a substituted phenyl group having a C₆-C₆₀ aryl group as a substituent.

The term “terphenyl group” as used herein refers to “a phenyl group substituted with a biphenyl group”. In other words, the “terphenyl group” is a substituted phenyl group having, as a substituent, a C₆-C₆₀ aryl group substituted with a C₆-C₆₀ aryl group.

* and *′ as used herein, unless defined otherwise, each refer to a binding site to a neighboring atom in a corresponding formula or moiety.

Hereinafter, a compound according to embodiments and a light-emitting device according to embodiments will be described in detail with reference to Synthesis Examples and Examples. The wording “B was used instead of A” used in describing Synthesis Examples refers to that an identical molar equivalent of B was used in place of A.

### EXAMPLES

### Synthesis Example 1: Synthesis of Compound 2

Compound 2 may be synthesized by, for example, Reaction Scheme 1 below.

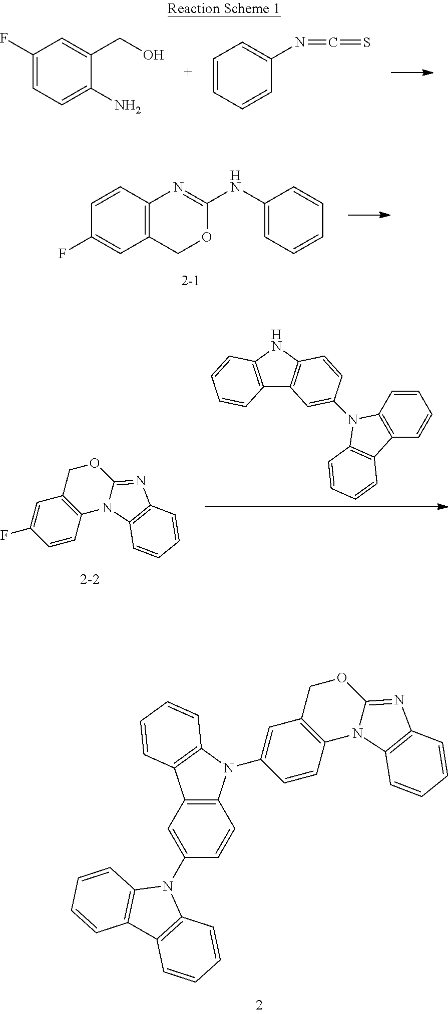 [SMILES: Nc1ccc(F)cc1CO ; S=C=Nc1ccccc1 ; Fc1ccc2c(c1)COC(Nc1ccccc1)=N2 ; Fc1ccc2c(c1)COc1nc3ccccc3n1-2 ; c1ccc2c(c1)[nH]c1ccc(-n3c4ccccc4c4ccccc43)cc12 ; c1ccc2c(c1)nc1n2-c2ccc(-n3c4ccccc4c4cc(-n5c6ccccc6c6ccccc65)ccc43)cc2CO1]

### (Synthesis of Intermediate 2-1)

(2-amino-5-fluorophenyl)methanol (CAS number=748805-85-8) was reacted with isothiocyanatobenzene (CAS number=103-72-0) in the presence of a Cu catalyst to obtain Intermediate 2-1. In relation to Intermediate 2-1, the following M+1 peak value was confirmed by liquid chromatography mass spectrometry (LC-MS).

C₁₄H₁₁FN₂O: M+1 243.11

### (Synthesis of Intermediate 2-2)

Intermediate 2-1 was reacted with iodine acid to obtain Intermediate 2-2. In relation to Intermediate 2-2, the following M+1 peak value was confirmed by liquid chromatography mass spectrometry (LC-MS).

C₁₄H₉FN₂O: M+1 241.05

### (Synthesis of Compound 2)

4 g of Intermediate 2-2, 5.5 g of 9H-3,9′-bicarbazole (CAS number=18628-07-4), 2.4 g of sodium tert-butoxide, 0.76 g of tris(dibenzylideneacetone)dipalladium (0), 0.7 mL of tri-tert-butylphosphine, and 85 mL of toluene were placed in a reaction vessel and refluxed for 24 hours. After the reaction was completed, the reaction solution was extracted with ethyl acetate, the collected organic layer was dried with magnesium sulfate and a solvent was evaporated therefrom. The obtained residue was separated and purified by silica gel column chromatography to obtain 7.1 g (yield: 78%) of Compound 2. Compound 2 was confirmed by LC-MS and ¹H-NMR.

### Synthesis Example 2: Synthesis of Compound 32

Compound 32 may be synthesized by, for example, Reaction Scheme 2 below.

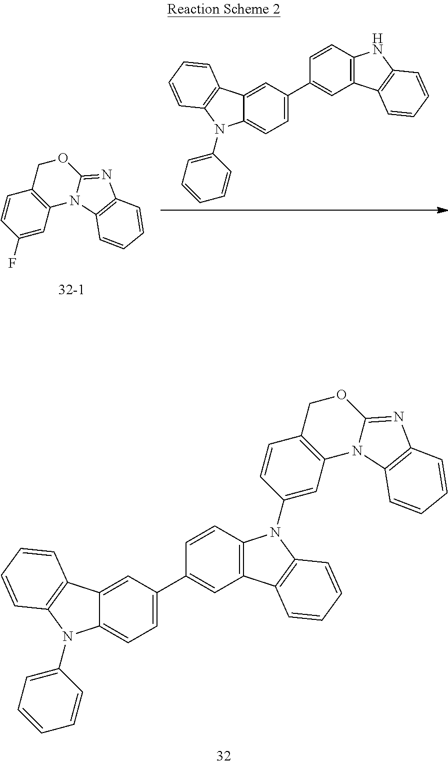 [SMILES: Fc1ccc2c(c1)-n1c(nc3ccccc31)OC2 ; c1ccc(-n2c3ccccc3c3cc(-c4ccc5[nH]c6ccccc6c5c4)ccc32)cc1 ; c1ccc(-n2c3ccccc3c3cc(-c4ccc5c(c4)c4ccccc4n5-c4ccc5c(c4)-n4c(nc6ccccc64)OC5)ccc32)cc1]

### (Synthesis of Intermediate 32-1)

Intermediate 32-1 was obtained in the same manner as used to synthesize Intermediate 2-2, except that (2-amino-4-fluorophenyl)methanol (CAS number=197783-88-3) was used instead of (2-amino-5-fluorophenyl)methanol (CAS number=748805-85-8). In relation to Intermediate 32-1, the following M+1 peak value was confirmed by liquid chromatography mass spectrometry (LC-MS).

C₁₄H₉FN₂O: M+1 241.09

### (Synthesis of Compound 32)

4.5 g of Intermediate 32-1, 7.7 g of 9-phenyl-3,3′-bi-9H-carbazole (CAS number=1060735-14-9), 2.7 g of sodium tert-butoxide, 0.85 g of tris(dibenzylideneacetone)dipalladium (0), 0.75 mL of tri tert-butylphosphine, and 95 mL of toluene were placed in a reaction vessel and refluxed for 24 hours. After the reaction was completed, the reaction solution was extracted with ethyl acetate, the collected organic layer was dried with magnesium sulfate and a solvent was evaporated therefrom. The obtained residue was separated and purified by silica gel column chromatography to 8.8 g (yield: 75%) of Compound 32. Compound 32 was confirmed by LC-MS and ¹H-NMR.

### Synthesis Example 3: Synthesis of Compound 128

Compound 128 may be synthesized by, for example, Reaction Scheme 3 below.

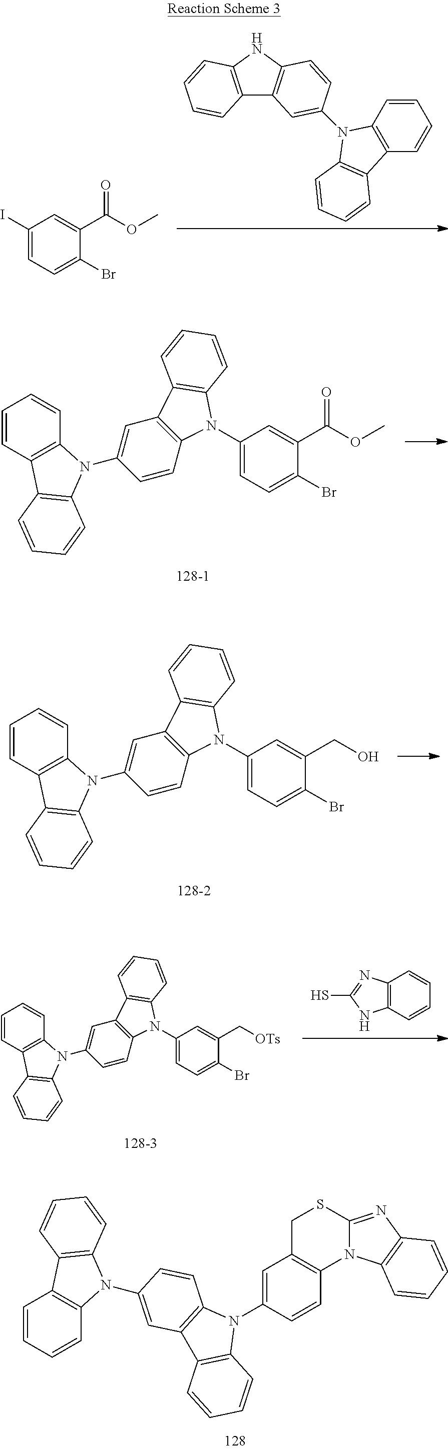

### (Synthesis of Intermediate 128-1)

Methyl 2-bromo-5-iodobenzoate (CAS number=717880-58-5) was reacted with 9H-3,9′-bicarbazole (CAS number=18628-07-4) in the presence of Cu to obtain Intermediate 128-1. In relation to Intermediate 128-1, the following M+1 peak value was confirmed by liquid chromatography mass spectrometry (LC-MS).

C₃₂H₂₁BrN₂O₂: M+1 545.11

### (Synthesis of Intermediate 128-2)

Intermediate 128-1 was reacted with lithium aluminum hydride to obtain Intermediate 128-2. In relation to Intermediate 128-2, the following M+1 peak value was confirmed by liquid chromatography mass spectrometry (LC-MS).

C₃₁H₂₁BrN₂O: M+1 517.09

### (Synthesis of Intermediate 128-3)

Intermediate 128-2 was tosylated with 4-toluenesulfonyl chloride (CAS number=98-59-9), and potassium hydroxide to obtain Intermediate 128-3. In relation to Intermediate 128-3, the following M+1 peak value was confirmed by liquid chromatography mass spectrometry (LC-MS).

C₃₈H₂₇BrN₂O₃S: M+1 671.07

### (Synthesis of Compound 128)

5 g of Intermediate 128-3, 1.2 g of 1H-benzimidazole-2-thiol, 0.14 g of copper(I) iodide, 6 g of cesium carbonate, 0.17 g of proline, and 120 mL of dimethylformamide were placed in a reaction vessel and refluxed for 24 hours. After the reaction was completed, the reaction solution was extracted with ethyl acetate, the collected organic layer was dried with magnesium sulfate and a solvent was evaporated therefrom. The obtained residue was separated and purified by silica gel column chromatography to 3.3 g (yield: 80%) of Compound 128. Compound 128 was confirmed by LC-MS and ¹H-NMR.

### Synthesis Example 4: Synthesis of Compound 176

Compound 176 may be synthesized by, for example, Reaction Scheme 4 below.

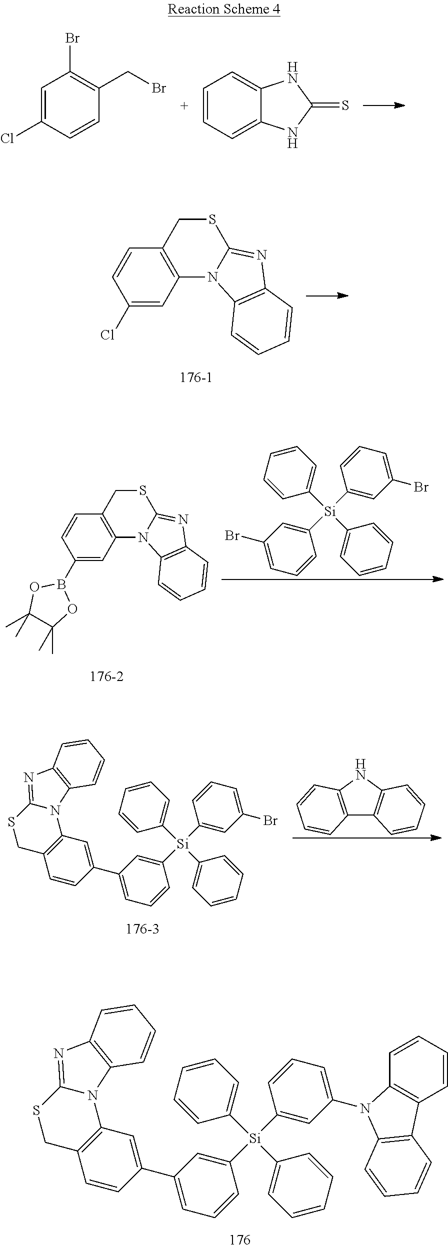

### (Synthesis of Intermediate 176-1)

2-bromo-1-(bromomethyl)-4-chloroa benzene (CAS number=33924-45-7) was reacted with 2-benzimidazolinethione (CAS number=583-39-1) in the presence of Cu to obtain Intermediate 176-1. In relation to Intermediate 176-1, the following M+1 peak value was confirmed by liquid chromatography mass spectrometry (LC-MS).

C₁₄H₉ClN₂S: M+1 273.01

### (Synthesis of Intermediate 176-2)

Intermediate 176-1 was reacted with bis(pinacolato)diboron in the presence of Pd catalyst to obtain Intermediate 176-2. In relation to Intermediate 176-2, the following M+1 peak value was confirmed by liquid chromatography mass spectrometry (LC-MS).

C₂₀H₂₁BN₂O₂S: M+1 365.17

### (Synthesis of Intermediate 176-3)

Intermediate 176-2 was reacted with bis(3-bromophenyl)diphenylsilane (CAS number=500886-98-6) in the presence of Pd catalyst to obtain Intermediate 176-3. In relation to Intermediate 176-3, the following M+1 peak value was confirmed by liquid chromatography mass spectrometry (LC-MS).

C₃₈H₂₇BrN₂SSi: M+1 651.08

### (Synthesis of Compound 176)

6 g of Intermediate 176-3, 1.5 g of 9H-carbazole (CAS number=86-74-8), 0.4 g of sodium tert-butoxide 1.3 g, tris(dibenzylideneacetone)dipalladium (0), 0.4 mL of tri tert-butylphosphine, 50 mL of toluene were placed in a reaction vessel and refluxed for 24 hours. After the reaction was completed, the reaction solution was extracted with ethyl acetate, the collected organic layer was dried with magnesium sulfate and a solvent was evaporated therefrom. The obtained residue was separated and purified by silica gel column chromatography to 5.6 g (yield: 83%) of Compound 176. Compound 176 was confirmed by LC-MS and ¹H-NMR.

### Synthesis Example 5: Synthesis of Compound 201

Compound 201 may be synthesized by, for example, Reaction Scheme 5 below.

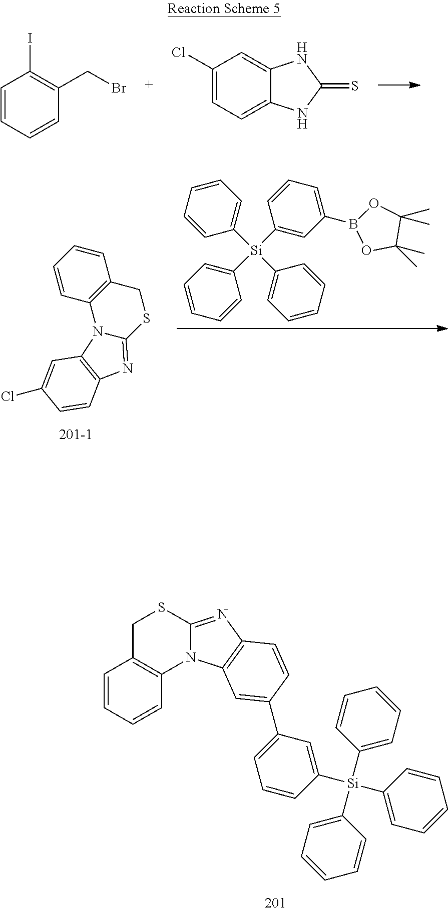

### (Synthesis of Intermediate 201-1)

1-(bromomethyl)-2-iodoa benzene (CAS number=40400-13-3) was reacted with 5-chloro-1,3-dihydro-2H-benzan imidazole-2-thione (CAS number=25369-78-2) to obtain Intermediate 201-1. In relation to Intermediate 201-1, the following M+1 peak value was confirmed by liquid chromatography mass spectrometry (LC-MS).

C₁₄H₉ClN₂S: M+1 273.02

### (Synthesis of Compound 201)

5 g of Intermediate 201-1, 8.5 g of 4,4,5,5-tetramethyl-2-[3-(triphenylsilyl)phenyl]-1,3,2-dioxoborolane (CAS number=1391041-75-0), 6.3 g of potassium carbonate, 1 g of tetrakis(triphenylphosphine)palladium (0), 100 mL of toluene, and 25 mL of distilled water were placed in a reaction vessel and refluxed for 24 hours. After the reaction was completed, the reaction solution was extracted with ethyl acetate, the collected organic layer was dried with magnesium sulfate and a solvent was evaporated therefrom. The obtained residue was separated and purified by silica gel column chromatography to obtain 7.3 g (yield: 70%) of Compound 201. Compound 201 was confirmed by LC-MS and ¹H-NMR.

### Synthesis Example 6: Synthesis of Compound 214

Compound 214 may be synthesized by, for example, Reaction Scheme 6 below.

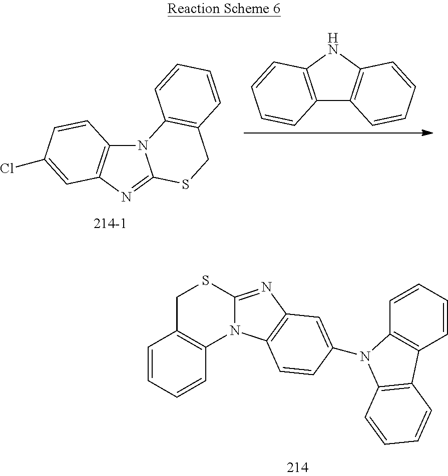

### (Synthesis of Intermediate 214-1)

Intermediate 214-1 was obtained in the same manner as used synthesize Intermediate 201-1. In relation to Intermediate 214-1, the following M+1 peak value was confirmed by liquid chromatography mass spectrometry (LC-MS).

C₁₄H₉ClN₂S: M+1 273.02

### (Synthesis of Compound 214)

4 g of Intermediate 214-1, 2.5 g of 9H-carbazole (CAS number=86-74-8), 2.1 g of sodium tert-butoxide, 0.67 g of tris(dibenzylideneacetone)dipalladium(0), 0.6 mL of tri tert-butylphosphine, and 75 mL of toluene were placed in a reaction vessel and refluxed for 24 hours. After the reaction was completed, the reaction solution was extracted with ethyl acetate, the collected organic layer was dried with magnesium sulfate and a solvent was evaporated therefrom. The obtained residue was separated and purified by silica gel column chromatography to obtain 4.3 g (yield: 73%) of Compound 214. Compound 214 was confirmed by LC-MS and ¹H-NMR.

### Synthesis Example 7: Synthesis of Compound 257

Compound 257 may be synthesized by, for example, Reaction Scheme 7 below.

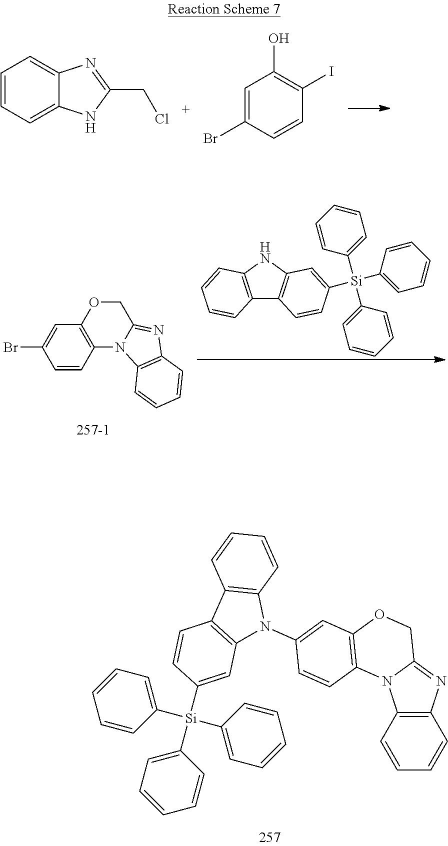 [SMILES: ClCc1nc2ccccc2[nH]1 ; Oc1cc(Br)ccc1I ; Brc1ccc2c(c1)OCc1nc3ccccc3n1-2 ; c1ccc([Si](c2ccccc2)(c2ccccc2)c2ccc3c(c2)[nH]c2ccccc23)cc1 ; c1ccc([Si](c2ccccc2)(c2ccccc2)c2ccc3c4ccccc4n(-c4ccc5c(c4)OCc4nc6ccccc6n4-5)c3c2)cc1]

### (Synthesis of Intermediate 257-1)

2-(chloromethyl)-1H-benzimidazole (CAS number=4857-04-9) was reacted with 5-bromo-2-iodophenol (CAS number=858855-11-5) in the presence of Cu catalyst to obtain Intermediate 257-1. In relation to Intermediate 257-1, the following M+1 peak value was confirmed by liquid chromatography mass spectrometry (LC-MS).

C₁₄H₉BrN₂O: M+1 300.99

### (Synthesis of Compound 257)

4 g of Intermediate 257-1, 5.7 g of 2-(triphenylsilyl)-9H-carbazole (CAS number=1262866-95-4), 1.9 g of sodium tert-butoxide, 0.6 g of tris(dibenzylideneacetone)dipalladium(0), 0.5 mL of tri tert-butylphosphine, and 70 mL of toluene were placed in a reaction vessel and refluxed for 24 hours. After the reaction was completed, the reaction solution was extracted with ethyl acetate, the collected organic layer was dried with magnesium sulfate and a solvent was evaporated therefrom. The obtained residue was separated and purified by silica gel column chromatography to 6.9 g (yield: 81%) of Compound 257. Compound 257 was confirmed by LC-MS and ¹H-NMR.

### Synthesis Example 8: Synthesis of Compound 289

Compound 289 may be synthesized by, for example, Reaction Scheme 8 below.

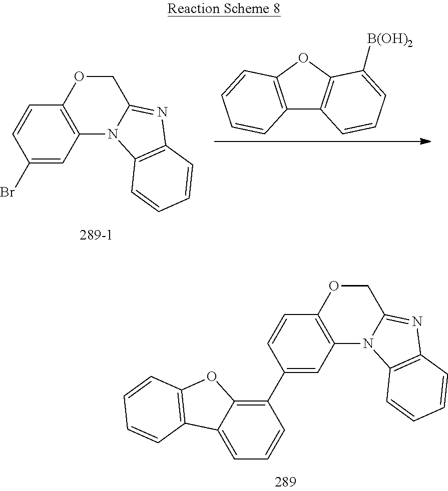 [SMILES: Brc1ccc2c(c1)-n1c(nc3ccccc31)CO2 ; OB(O)c1cccc2c1oc1ccccc12 ; c1ccc2c(c1)nc1n2-c2cc(-c3cccc4c3oc3ccccc34)ccc2OC1]

### (Synthesis of Intermediate 289-1)

Intermediate 289-1 was obtained in the same manner as used to synthesize Intermediate 257-1, except that 4-bromo-2-iodophenol (CAS number=207115-22-8) was used instead of 5-bromo-2-iodophenol (CAS number=858855-11-5). In relation to Intermediate 289-1, the following M+1 peak value was confirmed by liquid chromatography mass spectrometry (LC-MS).

C₁₄H₉BrN₂O: M+1 300.99

### (Synthesis of Compound 289)

4 g of Intermediate 289-1, 2.8 g of 4-dibenzofuranboronic acid (CAS number=100124-06-9), 4.6 g of potassium carbonate, 0.77 g of tetrakis(triphenylphosphine)palladium(0), 60 mL of toluene, and 15 mL of distilled water were placed in a reaction vessel and refluxed for 24 hours. After the reaction was completed, the reaction solution was extracted with ethyl acetate, the collected organic layer was dried with magnesium sulfate and a solvent was evaporated therefrom. The obtained residue was separated and purified by silica gel column chromatography to obtain 3.5 g (yield: 68%) of compound 289. Compound 289 was confirmed by LC-MS and ¹H-NMR.

### Synthesis Example 9: Synthesis of Compound 291

Compound 291 may be synthesized by, for example, Reaction Scheme 9 below.

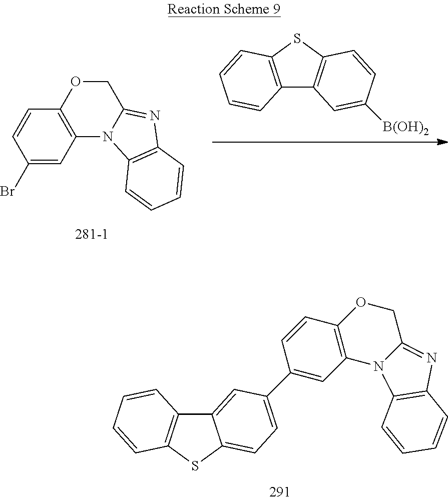 [SMILES: Brc1ccc2c(c1)-n1c(nc3ccccc31)CO2 ; OB(O)c1ccc2sc3ccccc3c2c1 ; c1ccc2c(c1)nc1n2-c2cc(-c3ccc4sc5ccccc5c4c3)ccc2OC1]

### (Synthesis of Compound 291)

The same manner as used to synthesize Compound 289 was used except that 2-dibenzothienylboronic acid (CAS number=668983-97-9) was used instead of 4-dibenzofuranboronic acid (CAS number=100124-06-9). 4 g (75%) of Compound 291 was obtained. Compound 291 was confirmed by LC-MS and ¹H-NMR.

¹H NMR and MS/FAB of the compounds synthesized according to Synthesis Examples 1 to 9 are shown in Table 1 below.

Synthesis methods for other compounds than the compounds shown in Table 1 may be easily recognized by those skilled in the technical field by referring to the synthesis paths and source material materials described above.

TABLE 1

| | | MS/FAB | |
|---|---|---|---|
| | | found | |
| Compound | ¹H NMR (CDCl₃, 400 MHz) | [M + 1] | calc. |
| Compound 2 | 8.55(m, 3H), 8.19(d, 1H), 7.94(d, 2H), 7.88(s, 1H), 7.78-7.67(m, | 553.21 | 552.20 |
| | 4H), 7.58-7.50(m, 3H), 7.38-7.16(m, 8H), 5.20(s, 2H) | | |
| Compound 32 | 8.56(m, 2H), 8.30(d, 1H), 8.19(d, 1H), 8.13(d, 1H), 7.99-7.89(m, | 629.19 | 628.23 |
| | 4H), 7.77(d, 1H), 7.62-7.16 (m, 16H), 5.20(s, 2H) | | |
| Compound | 8.55(m, 3H), 8.19(d, 1H), 7.94(d, 2H), 7.76-7.50(m, 8H), 7.38- | 569.20 | 568.17 |
| 128 | 7.16(m, 8H), 4.39(s, 2H) | | |
| Compound | 8.55(m, 2H), 8.19(d, 1H), 8.04(s, 1H), 7.94(d, 1H), 7.88(s, 1H), | 738.21 | 737.23 |
| 176 | 7.64-7.16(m, 27H), 4.39(s, 2H), | | |
| Compound | 8.19(d, 1H), 8.04(s, 1H), 7.88(s, 1H), 7.79(d, 1H), 7.64-7.56(m, 3H), | 573.15 | 572.17 |
| 201 | 7.46-7.38(m, 16H), 7.25(t, 1H), 7.16-7.10(m, 2H), 4.39(s, 2H) | | |
| Compound | 8.55(d, 1H), 8.19(d, 1H), 7.94(d, 1H), 7.82(s, 1H), 7.71(d, 1H), 7.58- | 404.10 | 403.11 |
| 214 | 7.46(m, 3H), 7.35-7.10(m, 7H), 4.39(s, 2H) | | |
| Compound | 8.55(m, 2H), 8.22(d, 1H), 7.94(d, 1H), 7.75(d, 1H), 7.68(s, 1H), | 646.23 | 645.22 |
| 257 | 7.54-7.16(m, 22H), 7.07(s, 1H), 5.20(s, 2H) | | |
| Compound | 8.56(d, 1H), 8.09-7.98(m, 4H), 7.71(d, 1H), 7.54-7.51(m,3H), 7.31- | 389.09 | 388.12 |
| 289 | 7.15(m, 5H), 5.20(s, 2H) | | |
| Compound | 8.56(d, 1H), 8.45(d, 1H), 8.12-8.09(m, 3H), 7.99(d, 1H), 7.93(d, | 405.12 | 404.10 |
| 291 | 1H), 7.71(d, 1H), 7.56-7.49(m, 3H), 7.28-7.15(m, 3H), 5.20(s, 2H) | | |

### Example 1

For a first electrode (anode), an ITO substrate having a thickness of 1200 Å was used. The ITO substrate was prepared by ultrasonic cleaning using isopropyl alcohol and pure water, each for 5 minutes, followed by irradiation with ultraviolet light for 30 minutes and exposure to ozone. The cleaned ITO substrate was mounted on a vacuum deposition apparatus.

N,N′-di(1-naphthyl)-N,N′-diphenylbenzidine (NPB) was vacuum-deposited on the ITO substrate, which has been prepared by cleaning, to form a hole injection layer having a thickness of 300 Å. mCP was vacuum-deposited on the hole injection layer to form a hole transport layer having a thickness of 200 Å.

Compound 2 (host) and Ir(pmp)₃(dopant) were co-deposited at the weight ratio of 92:8 on the hole transport layer to form an emission layer having a thickness of 250 Å.

Then, 3-(4-biphenylyl)-4-phenyl-5-tert-butylphenyl-1,2,4-triazole (TAZ) was deposited on the emission layer to form an electron transport layer having a thickness of 200 Å. LiF, which is a halogenated alkali metal, was deposited on the electron transport layer to form an electron injection layer having a thickness of 10 Å, and Al was vacuum-deposited on the electron injection layer to form a second electrode (cathode) having a thickness of 100 Å, thereby completing the formation of an LiF/Al electrode.

Materials used in the above-described organic light-emitting device may be represented by the formula below.

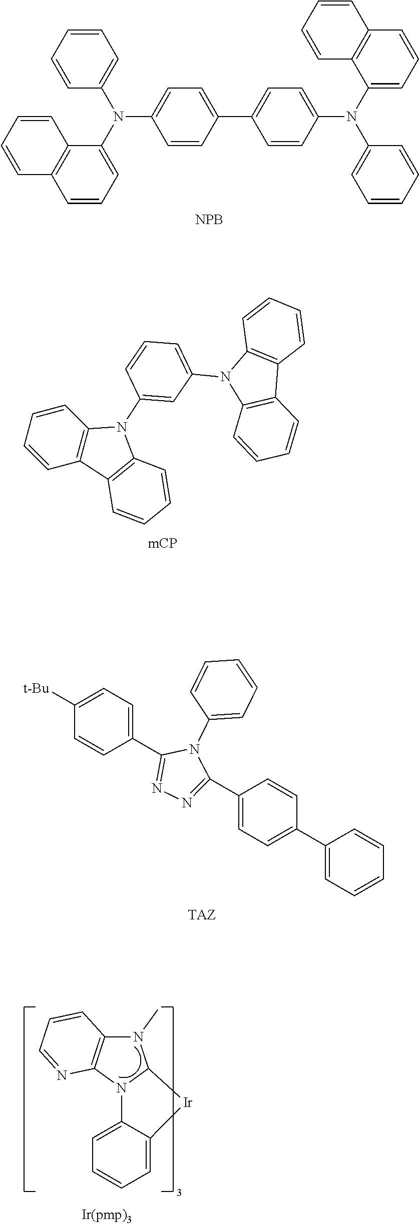

### Examples 2 to 9

Organic light-emitting devices were manufactured in the same manner as in Example 1, except that the compounds of Table 2 were used to form emission layers.

### Comparative Examples 1 and 2

Organic light-emitting devices were manufactured in the same manner as in Example 1, except that compounds C1 and C2 were used to form emission layers.

### Evaluation Example: Evaluation of OLED Characteristics

In order to evaluate the characteristics of the organic light-emitting devices manufactured in Examples 1 to 9 and Comparative Examples 1 and 2, the driving voltage, current efficiency, and maximum quantum efficiency thereof at the current density of 2.3 mA/cm² were measured. The driving voltage and current density of the organic light-emitting devices were measured using a source meter (Keithley Instrument, 2400 series), and the maximum quantum efficiency was measured using the external quantum efficiency measurement device C9920-2-12 of Hamamatsu Photonics Inc. In evaluating the maximum quantum efficiency, the luminance/current density was measured using a luminance meter that was calibrated for wavelength sensitivity, and the maximum quantum efficiency was converted by assuming an angular luminance distribution (Lambertian) which introduced a perfect reflecting diffuser. Table 2 below shows the evaluation results of the characteristics of the organic light-emitting devices.

TABLE 2

| | Host of | Driving | Current density | Maximum quantum | |
|---|---|---|---|---|---|
| | emission layer | voltage (V) | (mA/cm²) | efficiency (%) | Emission color |
| Example 1 | Compound 2 | 4.5 | 2.3 | 20.9 | Blue |
| Example 2 | Compound 32 | 4.3 | 2.3 | 19.8 | Blue |
| Example 3 | Compound 128 | 4.5 | 2.3 | 21.5 | Blue |
| Example 4 | Compound 176 | 4.6 | 2.3 | 21.7 | Blue |
| Example 5 | Compound 201 | 4.7 | 2.3 | 19.3 | Blue |
| Example 6 | Compound 214 | 4.4 | 2.3 | 22.8 | Blue |
| Example 7 | Compound 257 | 4.7 | 2.3 | 20.7 | Blue |
| Example 8 | Compound 289 | 4.9 | 2.3 | 18.7 | Blue |
| Example 9 | Compound 291 | 4.8 | 2.3 | 20.1 | Blue |
| Comparative | Compound C1 | 4.8 | 2.3 | 17.7 | Blue |
| Example 1 | | | | | |
| Comparative | Compound C2 | 5.0 | 2.3 | 18.1 | Blue |
| Example 2 | | | | | |

| | |
|---|---|
| 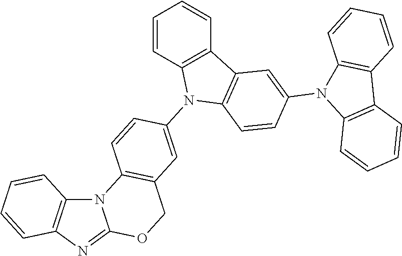 [SMILES: c1ccc2c(c1)nc1n2-c2ccc(-n3c4ccccc4c4cc(-n5c6ccccc6c6ccccc65)ccc43)cc2CO1] | 2 |
| 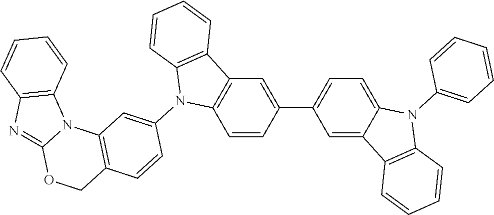 [SMILES: c1ccc(-n2c3ccccc3c3cc(-c4ccc5c(c4)c4ccccc4n5-c4ccc5c(c4)-n4c(nc6ccccc64)OC5)ccc32)cc1] | 32 |
| 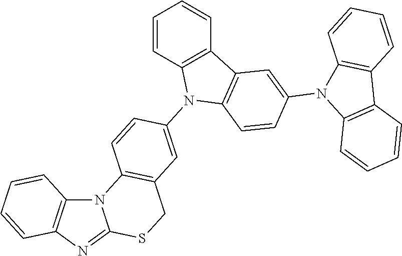 [SMILES: c1ccc2c(c1)nc1n2-c2ccc(-n3c4ccccc4c4cc(-n5c6ccccc6c6ccccc65)ccc43)cc2CS1] | 128 |
| 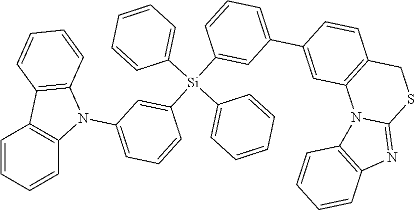 [SMILES: c1ccc([Si](c2ccccc2)(c2cccc(-c3ccc4c(c3)-n3c(nc5ccccc53)SC4)c2)c2cccc(-n3c4ccccc4c4ccccc43)c2)cc1] | 176 |
| 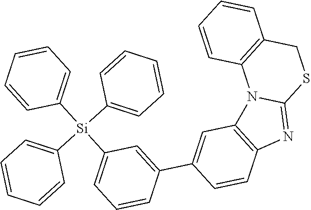 [SMILES: c1ccc([Si](c2ccccc2)(c2ccccc2)c2cccc(-c3ccc4nc5n(c4c3)-c3ccccc3CS5)c2)cc1] | 201 |
| 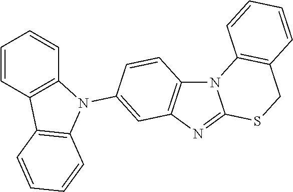 [SMILES: c1ccc2c(c1)CSc1nc3cc(-n4c5ccccc5c5ccccc54)ccc3n1-2] | 214 |
| 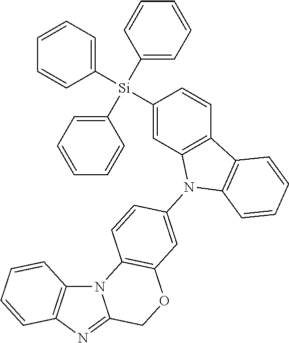 [SMILES: c1ccc([Si](c2ccccc2)(c2ccccc2)c2ccc3c4ccccc4n(-c4ccc5c(c4)OCc4nc6ccccc6n4-5)c3c2)cc1] | 257 |
| 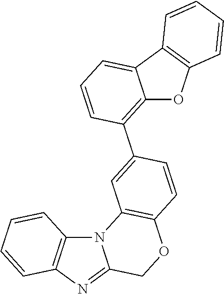 [SMILES: c1ccc2c(c1)nc1n2-c2cc(-c3cccc4c3oc3ccccc34)ccc2OC1] | 289 |
| 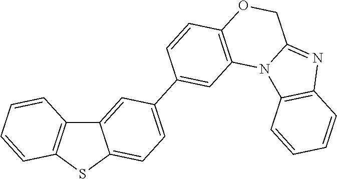 [SMILES: c1ccc2c(c1)nc1n2-c2cc(-c3ccc4sc5ccccc5c4c3)ccc2OC1] | 291 |
| 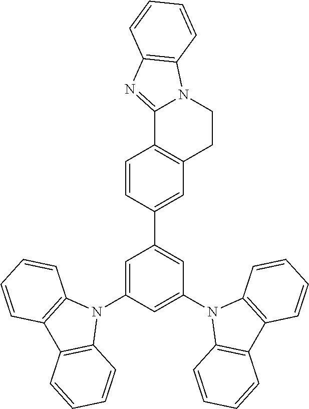 [SMILES: c1ccc2c(c1)nc1n2CCc2cc(-c3cc(-n4c5ccccc5c5ccccc54)cc(-n4c5ccccc5c5ccccc54)c3)ccc2-1] | C1 |
| 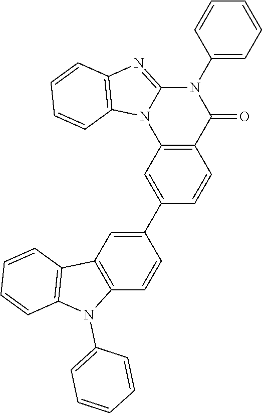 [SMILES: O=c1c2ccc(-c3ccc4c(c3)c3ccccc3n4-c3ccccc3)cc2n2c3ccccc3nc2n1-c1ccccc1] | C2 |

Referring to the results of Table 2, it can be seen that the organic light-emitting devices of Examples 1 to 9 have a low driving voltage and high maximum quantum efficiency compared to the organic light-emitting devices of Comparative Examples 1 and 2.

A light-emitting device including the heterocyclic compound according to an embodiment of the present disclosure may have a low driving voltage, improved efficiency and/or improved lifespan.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A light-emitting device comprising:
a first electrode;
a second electrode; and
an interlayer located between the first electrode and the second electrode and comprising an emission layer, wherein
at least one heterocyclic compound represented by Formula 1: 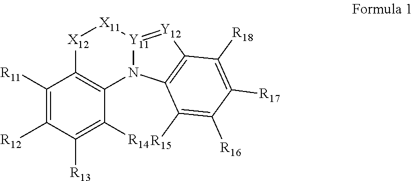
wherein, in Formula 1,
i) X₁₁ is O or S and X₁₂ is C(R₁₉)(R₂₀), or ii) X₁₁ is C(R₁₉)(R₂₀) and X₁₂ is O or S;
Y₁₁ and Y₁₂ are each N or C,
R₁₁ to R₂₀ are each independently a group represented by *-(L₂₁)ₐ₂₁-(R₂₁)_{b21}, hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂), wherein at least one of R₁₁ to R₂₀ is a group represented by *-(L₂₁)ₐ₂₁-(R₂₁)_{b21},
L₂₁ is a single bond, *—N(R₃₁)—*′, *—Si(R₃₁)(R₃₂)—*′, a C₅-C₆₀ carbocyclic group unsubstituted or substituted with at least one R₁₀ₐ, or a C₁-C₆₀ heterocyclic group unsubstituted or substituted with at least one R₁₀ₐ,
a21 is an integer selected from 1 to 5,
R₂₁ is a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
b21 is an integer selected from 1 to 5,
R₃₁ and R₃₂ are each independently hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
R₁₀ₐ is:
deuterium (-D), —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —C(Q₁₁)(Q₁₂)(Q₁₃), —B(Q₁₁)(Q₁₂), —N(Q₁₁)(Q₁₂), —P(Q₁₁)(Q₁₂), —C(═O)(Q₁₁), —S(═O)(Q₁₁), —S(═O)₂(Q₁₁), —P(═O)(Q₁₁)(Q₁₂), —P(═S)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —C(Q₂₁)(Q₂₂)(Q₂₃), —B(Q₂₁)(Q₂₂), —N(Q₂₁)(Q₂₂), —P(Q₂₁)(Q₂₂), —C(═O)(Q₂₁), —S(═O)(Q₂₁), —S(═O)₂(Q₂₁), —P(═O)(Q₂₁)(Q₂₂) or —P(═S)(Q₂₁)(Q₂₂), or any combination thereof; or
—Si(Q₃₁)(Q₃₂)(Q₃₃), —C(Q₃₁)(Q₃₂)(Q₃₃), —B(Q₃₁)(Q₃₂), —N(Q₃₁)(Q₃₂), —P(Q₃₁)(Q₃₂), —C(═O)(Q₃₁), —S(═O)(Q₃₁), —S(═O)₂(Q₃₁), —P(═O)(Q₃₁)(Q₃₂), or —P(═S)(Q₃₁)(Q₃₂),
wherein Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and
* and *′ each indicate a binding site to a neighboring atom.

2. The light-emitting device of claim 1, wherein the first electrode comprises indium tin oxide (ITO), indium zinc oxide (IZO), tin oxide (SnO₂), zinc oxide (ZnO), magnesium (Mg), silver (Ag), aluminum (Al), aluminum-lithium (Al—Li), calcium (Ca), magnesium-indium (Mg—In), magnesium-silver (Mg—Ag), or any combination thereof, and
the second electrode comprises lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof.

3. The light-emitting device of claim 1, wherein the first electrode of the light-emitting device is an anode,
the second electrode of the light-emitting device is a cathode,
the interlayer further comprises a hole transport region between the first electrode and the emission layer and an electron transport region between the emission layer and the second electrode,
the hole transport region comprises a hole injection layer, a hole transport layer, an emission auxiliary layer, an electron blocking layer, or any combination thereof, and
the electron transport region comprises a buffer layer, a hole blocking layer, an electron control layer, an electron transport layer, an electron injection layer, or any combination thereof.

4. The light-emitting device of claim 1, wherein the emission layer comprises the at least one heterocyclic compound represented by Formula 1.

5. The light-emitting device of claim 4, wherein the emission layer comprises a host and a dopant,
an amount of the host in the emission layer is greater than an amount of the dopant in the emission layer,
the host comprises the at least one heterocyclic compound represented by Formula 1, and
the dopant comprises a phosphorescent dopant, a thermally activated delayed fluorescence dopant, or a combination thereof.

6. The light-emitting device of claim 4, wherein the emission layer emits blue light.

7. The light-emitting device of claim 1, further comprising a capping layer located outside the first electrode and/or outside the second electrode,
wherein the capping layer comprises the at least one heterocyclic compound represented by Formula 1.

8. An electronic apparatus comprising the light-emitting device of claim 1 and a thin-film transistor,
wherein the thin-film transistor comprises a source electrode and a drain electrode, and the first electrode of the light-emitting device is electrically connected to one of the source electrode and the drain electrode of the thin-film transistor.

9. A heterocyclic compound represented by Formula 1: 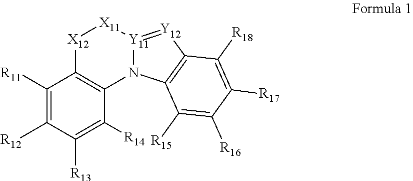
wherein, in Formula 1,
i) X₁₁ is O or S and X₁₂ is C(R₁₉)(R₂₀), or ii) X₁₁ is C(R₁₉)(R₂₀) and X₁₂ is O or S;
Y₁₁ and Y₁₂ are each N or C,
R₁₁ to R₂₀ are each independently a group represented by *-(L₂₁)ₐ₂₁-(R₂₁)_{b21}, hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂), wherein at least one of R₁₁ to R₂₀ is a group represented by *-(L₂₁)ₐ₂₁-(R₂₁)_{b21},
L₂₁ is a single bond, *—N(R₃₁)—*′, *—Si(R₃₁)(R₃₂)—*′, a C₅-C₆₀ carbocyclic group unsubstituted or substituted with at least one R₁₀ₐ, or a C₁-C₆₀ heterocyclic group unsubstituted or substituted with at least one R₁₀ₐ,
a21 is an integer selected from 1 to 5,
R₂₁ is a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
b21 is an integer selected from 1 to 5,
R₃₁ and R₃₂ are each independently hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
R₁₀ₐ is:
deuterium (-D), —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —C(Q₁₁)(Q₁₂)(Q₁₃), —B(Q₁₁)(Q₁₂), —N(Q₁₁)(Q₁₂), —P(Q₁₁)(Q₁₂), —C(═O)(Q₁₁), —S(═O)(Q₁₁), —S(═O)₂(Q₁₁), —P(═O)(Q₁₁)(Q₁₂), —P(═S)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —C(Q₂₁)(Q₂₂)(Q₂₃), —B(Q₂₁)(Q₂₂), —N(Q₂₁)(Q₂₂), —P(Q₂₁)(Q₂₂), —C(═O)(Q₂₁), —S(═O)(Q₂₁), —S(═O)₂(Q₂₁), —P(═O)(Q₂₁)(Q₂₂) or —P(═S)(Q₂₁)(Q₂₂), or any combination thereof; or
—Si(Q₃₁)(Q₃₂)(Q₃₃), —C(Q₃₁)(Q₃₂)(Q₃₃), —B(Q₃₁)(Q₃₂), —N(Q₃₁)(Q₃₂), —P(Q₃₁)(Q₃₂), —C(═O)(Q₃₁), —S(═O)(Q₃₁), —S(═O)₂(Q₃₁), —P(═O)(Q₃₁)(Q₃₂), or —P(═S)(Q₃₁)(Q₃₂),
wherein Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and
* and *′ each indicate a binding site to a neighboring atom.

10. The heterocyclic compound of claim 9, wherein each of Y₁₁ and Y₁₂ is N;
each of Y₁₁ and Y₁₂ is C; or
one of Y₁₁ and Y₁₂ is N and the other one is C.

11. The heterocyclic compound of claim 9, wherein Y₁₁ is C and Y₁₂ is N.

12. The heterocyclic compound of claim 9, wherein one or two of R₁₁ to R₁₈ are represented by *-(L₂₁)ₐ₂₁-(R₂₁)_{b21}.

13. The heterocyclic compound of claim 9, wherein one of R₁₁ to R₁₈ is represented by *-(L₂₁)ₐ₂₁-(R₂₁)_{b21}.

14. The heterocyclic compound of claim 9, wherein R₁₂, R₁₃, R₁₆, or R₁₇ is a group represented by *-(L₂₁)ₐ₂₁-(R₂₁)_{b21}.

15. The heterocyclic compound of claim 9, wherein L₂₁ is:
a single bond or *—Si(R₃₁)(R₃₂)—*′, or
a benzene group, a pentalene group, an indene group, a naphthalene group, an azulene group, a heptalene group, an indacene group, an acenaphthalene group, a fluorene group, a spiro-bifluorene group, a spiro-benzofluorene-fluorene group, a benzofluorene group, a dibenzofluorene group, a phenalene group, a phenanthrene group, an anthracene group, a fluoranthene group, a pyrene group, a chrysene group, a naphthacene group, a picene group, a perylene group, a pyrrole group, a thiophene group, a furan group, a silole group, an imidazole group, a pyrazole group, a thiazole group, an isothiazole group, an oxazole group, an isoxazole group, a pyridine group, a pyrazine group, a pyrimidine group, a pyridazine group, a triazine group, a benzofuran group, a benzothiophene group, a dibenzofuran group, a dibenzothiophene group, a carbazole group, a benzosilole group, a dibenzosilole group, a quinoline group, an isoquinoline group, a benzimidazole group, an imidazopyridine group, or an imidazopyrimidine group, each unsubstituted or substituted with deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a spiro-benzofluorene-fluorenyl group, a benzofluorenyl group, a dibenzofluorenyl group, a phenalenyl group, a phenanthrenyl group, an anthracenyl group, a fluoranthenyl group, a pyrenyl group, a chrysenyl group, a naphthacenyl group, a picenyl group, a perylenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, a benzofuranyl group, a benzothiophenyl group, a dibenzofuranyl group, a dibenzothiophenyl group, a carbazolyl group, a benzosilolyl group, a dibenzosilolyl group, a quinolinyl group, an isoquinolinyl group, a benzimidazolyl group, an imidazopyridinyl group, an imidazopyrimidinyl group, —Si(Q₃₁)(Q₃₂)(Q₃₃), —C(Q₃₁)(Q₃₂)(Q₃₃), —B(Q₃₁)(Q₃₂), —N(Q₃₁)(Q₃₂), —P(Q₃₁)(Q₃₂), —C(═O)(Q₃₁), —S(═O)(Q₃₁), —S(═O)₂(Q₃₁), —P(═O)(Q₃₁)(Q₃₂), —P(═S)(Q₃₁)(Q₃₂), or any combination thereof,
R₃₁ and R₃₂ are each independently hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a spiro-benzofluorene-fluorenyl group, a benzofluorenyl group, a dibenzofluorenyl group, a phenalenyl group, a phenanthrenyl group, an anthracenyl group, a fluoranthenyl group, a pyrenyl group, a chrysenyl group, a naphthacenyl group, a picenyl group, a perylenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, a benzofuranyl group, a benzothiophenyl group, a dibenzofuranyl group, a dibenzothiophenyl group, a carbazolyl group, a benzosilolyl group, a dibenzosilolyl group, a quinolinyl group, an isoquinolinyl group, a benzimidazolyl group, an imidazopyridinyl group, an imidazopyrimidinyl group, —Si(Q₃₁)(Q₃₂)(Q₃₃), —C(Q₃₁)(Q₃₂)(Q₃₃), —B(Q₃₁)(Q₃₂), —N(Q₃₁)(Q₃₂), —P(Q₃₁)(Q₃₂), —C(═O)(Q₃₁), —S(═O)(Q₃₁), —S(═O)₂(Q₃₁), —P(═O)(Q₃₁)(Q₃₂), or —P(═S)(Q₃₁)(Q₃₂), and
R₂₁ is: a cyclopentenyl group, a cyclohexenyl group, a phenyl group, a biphenyl group, a terphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, an indolyl group, an isoindolyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a dibenzofuranyl group, a dibenzothiophenyl group, a dibenzosilolyl group, a carbazolyl group, a benzocarbazolyl group, a dibenzocarbazolyl group, an azafluorenyl group, an azaspiro-bifluorenyl group, a dibenzothiophenylfluorenyl group, an azacarbazolyl group, a diazacarbazolyl group, an azadibenzofuranyl group, an azadibenzothiophenyl group, an azadibenzosilolyl group, an imidazopyridinyl group, or an imidazopyrimidinyl group, each unsubstituted or substituted with deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkyl group substituted with at least one phenyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, an indolyl group, an isoindolyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a dibenzofuranyl group, a dibenzothiophenyl group, a dibenzosilolyl group, a carbazolyl group, a benzocarbazolyl group, a dibenzocarbazolyl group, an azafluorenyl group, an azaspiro-bifluorenyl group, a dibenzothiophenylfluorenyl group, an azacarbazolyl group, a diazacarbazolyl group, an azadibenzofuranyl group, an azadibenzothiophenyl group, an azadibenzosilolyl group, an imidazopyridinyl group, an imidazopyrimidinyl group, —Si(Q₃₁)(Q₃₂)(Q₃₃), —C(Q₃₁)(Q₃₂)(Q₃₃), —N(Q₃₁)(Q₃₂), —B(Q₃₁)(Q₃₂), or any combination thereof; or
—Si(Q₁)(Q₂)(Q₃), —C(Q₁)(Q₂)(Q₃), —N(Q₁)(Q₂), or —B(Q₁)(Q₂),
wherein Q₁ to Q₃ and Q₃₁ to Q₃₃ are each independently a C₁-C₁₀ alkyl group, a C₁-C₁₀ alkoxy group, a phenyl group, a phenyl group substituted with a cyano group, a biphenyl group, a terphenyl group, or a naphthyl group.

16. The heterocyclic compound of claim 9, wherein R₂₁ is a group represented by one of Formulae 5-1 to 5-20: 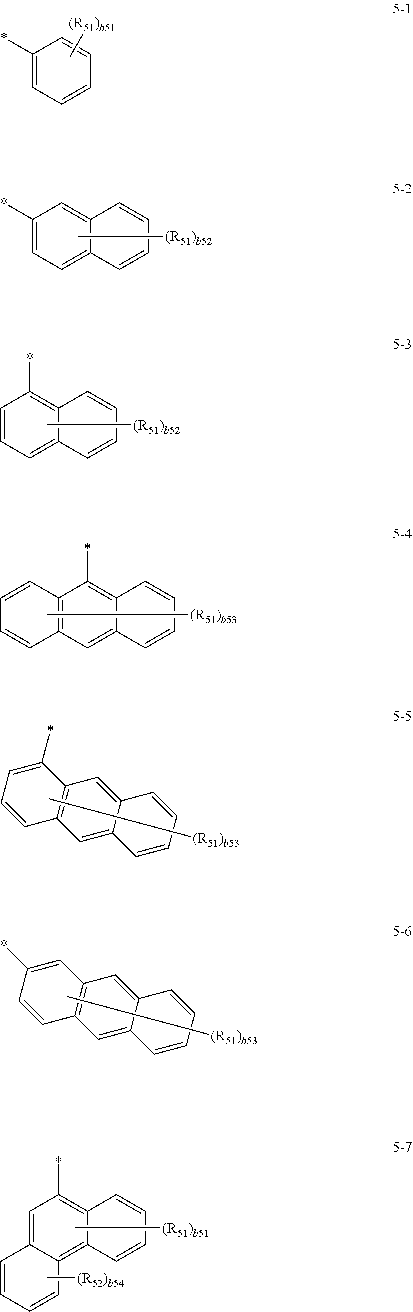 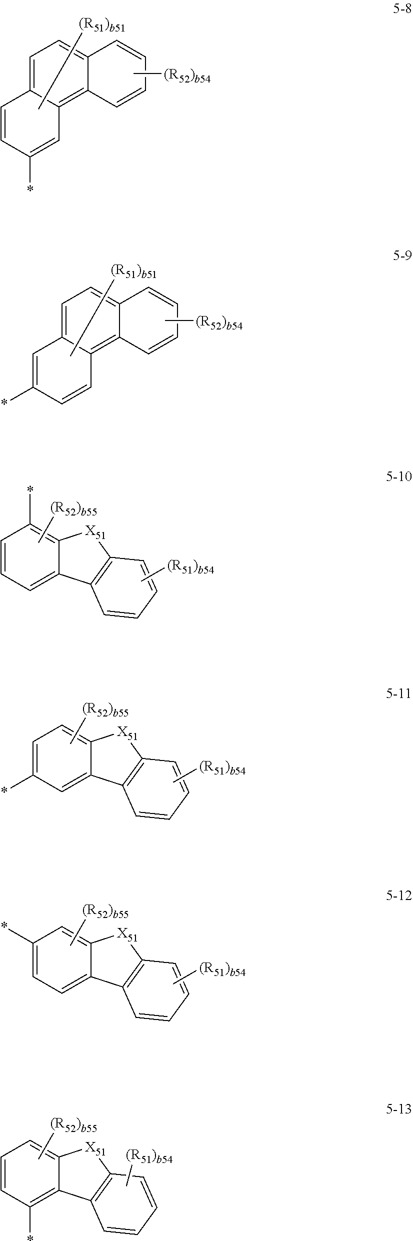 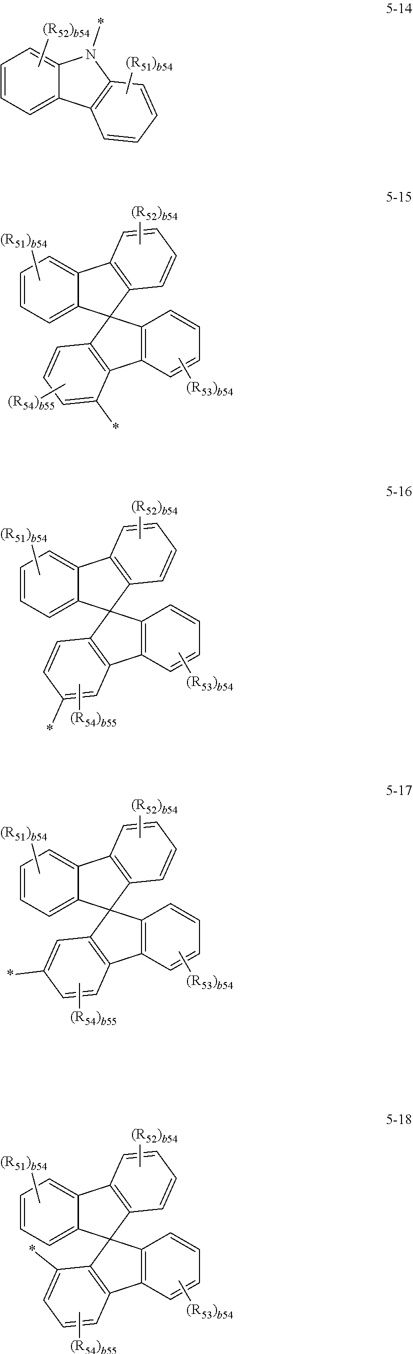 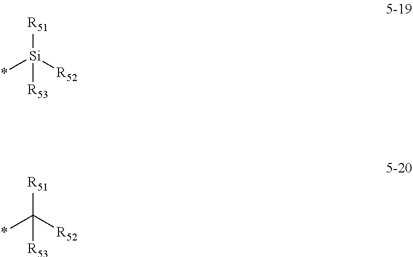
wherein, in Formulae 5-1 to 5-20,
X₅₁ is O, S, N(R₅₃), or C(R₅₃)(R₅₄),
R₅₁ to R₅₄ are each independently hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkyl group substituted with at least one phenyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyridinyl group, a pyrimidinyl group, a pyrazinyl group, a triazinyl group, a quinolinyl group, an isoquinolinyl group, a benzoquinolinyl group, a naphthyridinyl group, a quinoxalinyl group, a quinazolinyl group, a carbazolyl group, a phenanthridinyl group, an acridinyl group, a phenanthrolinyl group, a phenazinyl group, a dibenzofuranyl group, a dibenzothiophenyl group, a dibenzosilolyl group, —Si(Q₃₁)(Q₃₂)(Q₃₃), —C(Q₃₁)(Q₃₂)(Q₃₃), —N(Q₃₁)(Q₃₂), or —B(Q₃₁)(Q₃₂),
b51 is an integer selected from 1 to 5,
b52 is an integer selected from 1 to 7,
b53 is an integer selected from 1 to 9,
b54 is an integer selected from 1 to 4,
b55 is an integer selected from 1 to 3,
Q₃₁ to Q₃₃ are each independently a C₁-C₁₀ alkyl group, a C₁-C₁₀ alkoxy group, a phenyl group, a phenyl group substituted with a cyano group, a biphenyl group, a terphenyl group, or a naphthyl group, and
* indicates a binding site to an adjacent atom.

17. The heterocyclic compound of claim 9, wherein
R₁₁ to R₂₀ are each independently a group represented by *-(L₂₁)ₐ₂₁-(R₂₁)_{b21}, hydrogen, deuterium, —F, —Cl, —Br, —I, a cyano group, a C₁-C₂₀ alkyl group, a C₁-C₂₀ alkyl group substituted with at least one phenyl group, or a C₁-C₂₀ alkoxy group;
a cyclopentyl group, a cyclohexyl group, a cycloheptyl group, a cyclopentenyl group, a cyclohexenyl group, a phenyl group, a biphenyl group, a terphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, an indolyl group, an isoindolyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a dibenzofuranyl group, a dibenzothiophenyl group, a dibenzosilolyl group, a carbazolyl group, a benzocarbazolyl group, a dibenzocarbazolyl group, an azafluorenyl group, an azaspiro-bifluorenyl group, a dibenzothiophenylfluorenyl group, an azacarbazolyl group, a diazacarbazolyl group, an azadibenzofuranyl group, an azadibenzothiophenyl group, an azadibenzosilolyl group, an imidazopyridinyl group, or an imidazopyrimidinyl group, each unsubstituted or substituted with deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkyl group substituted with at least one phenyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, an indolyl group, an isoindolyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a dibenzofuranyl group, a dibenzothiophenyl group, a dibenzosilolyl group, a carbazolyl group, a benzocarbazolyl group, a dibenzocarbazolyl group, an azafluorenyl group, an azaspiro-bifluorenyl group, a dibenzothiophenylfluorenyl group, an azacarbazolyl group, a diazacarbazolyl group, an azadibenzofuranyl group, an azadibenzothiophenyl group, an azadibenzosilolyl group, an imidazopyridinyl group, an imidazopyrimidinyl group, —Si(Q₃₁)(Q₃₂)(Q₃₃), —C(Q₃₁)(Q₃₂)(Q₃₃), —N(Q₃₁)(Q₃₂), —B(Q₃₁)(Q₃₂), or any combination thereof; or
—Si(Q₁)(Q₂)(Q₃), —C(Q₁)(Q₂)(Q₃), —N(Q₁)(Q₂), or —B(Q₁)(Q₂),
wherein Q₁ to Q₃ and Q₃₁ to Q₃₃ are each independently a C₁-C₁₀ alkyl group, a C₁-C₁₀ alkoxy group, a phenyl group, a phenyl group substituted with a cyano group, a biphenyl group, a terphenyl group, or a naphthyl group.

18. The heterocyclic compound of claim 9, wherein R₁₁ to R₂₀ are each independently a group represented by *-(L₂₁)ₐ₂₁-(R₂₁)_{b21}, hydrogen, deuterium, —F, —Cl, —Br, —I, a cyano group, a C₁-C₂₀ alkyl group, or a group represented by one of Formulae 5-1 to 5-20: 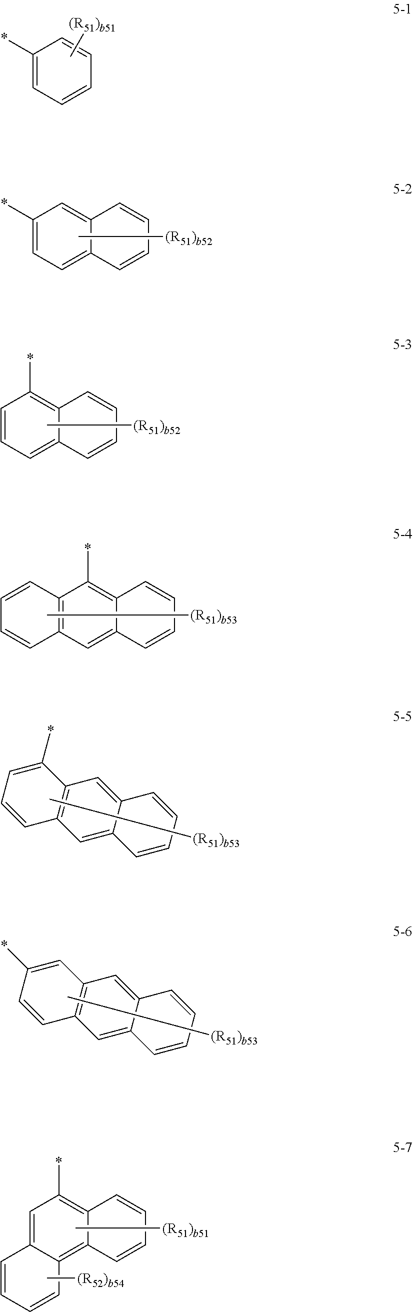 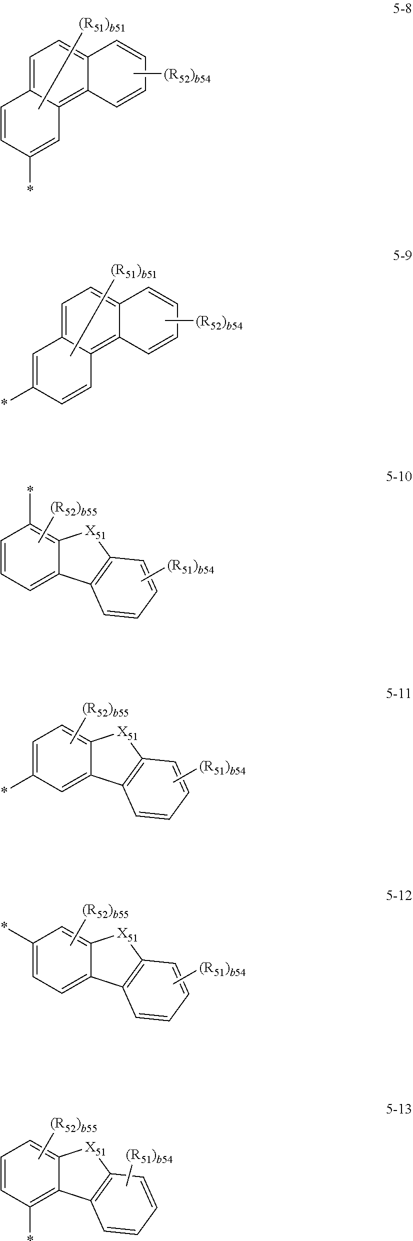 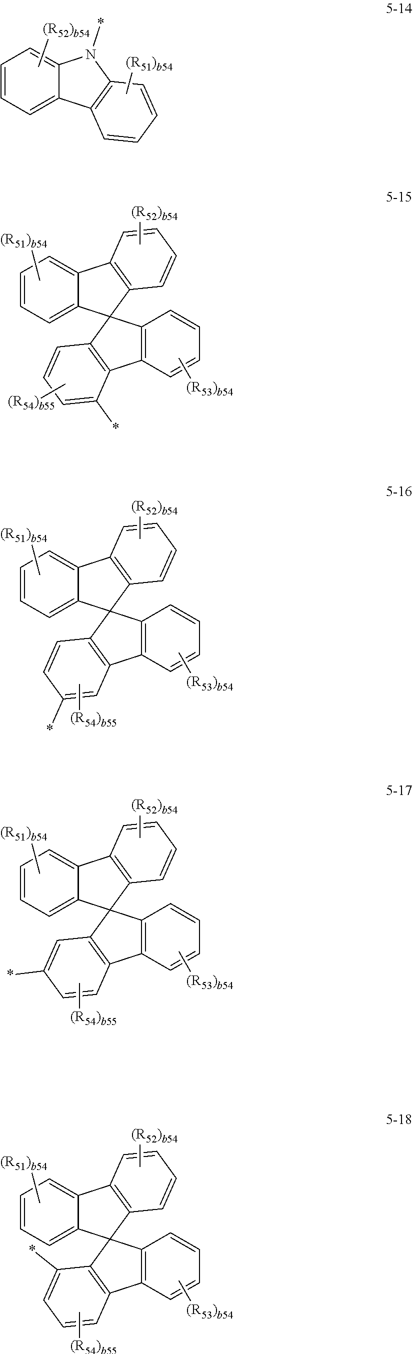 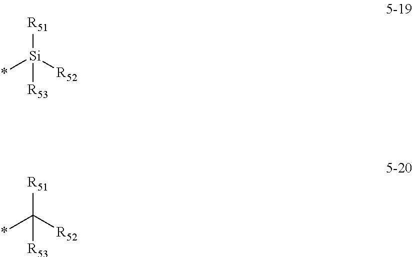
wherein, in Formulae 5-1 to 5-20,
X₅₁ is O, S, N(R₅₃), or C(R₅₃)(R₅₄),
R₅₁ to R₅₄ are each independently hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkyl group substituted with at least one phenyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyridinyl group, a pyrimidinyl group, a pyrazinyl group, a triazinyl group, a quinolinyl group, an isoquinolinyl group, a benzoquinolinyl group, a naphthyridinyl group, a quinoxalinyl group, a quinazolinyl group, a carbazolyl group, a phenanthridinyl group, an acridinyl group, a phenanthrolinyl group, a phenazinyl group, a dibenzofuranyl group, a dibenzothiophenyl group, a dibenzosilolyl group, —Si(Q₃₁)(Q₃₂)(Q₃₃), —C(Q₃₁)(Q₃₂)(Q₃₃), —N(Q₃₁)(Q₃₂), or —B(Q₃₁)(Q₃₂),
b51 is an integer selected from 1 to 5,
b52 is an integer selected from 1 to 7,
b53 is an integer selected from 1 to 9,
b54 is an integer selected from 1 to 4,
b55 is an integer selected from 1 to 3,
Q₃₁ to Q₃₃ are each independently a C₁-C₁₀ alkyl group, a C₁-C₁₀ alkoxy group, a phenyl group, a phenyl group substituted with a cyano group, a biphenyl group, a terphenyl group, or a naphthyl group, and
* indicates a binding site to an adjacent atom.

19. The heterocyclic compound of claim 9, wherein the at least one heterocyclic compound is represented by one of Formulae 1-1 to 1-8: 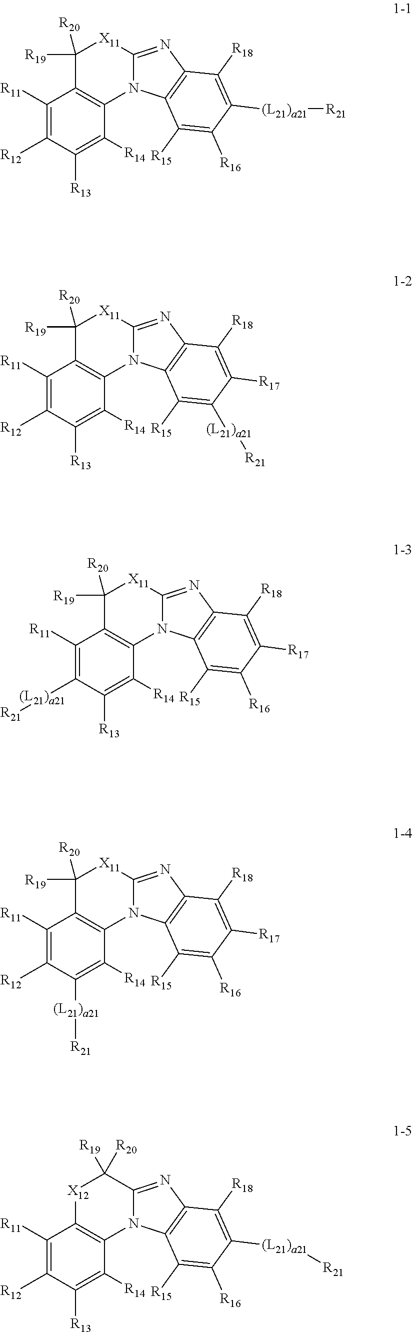 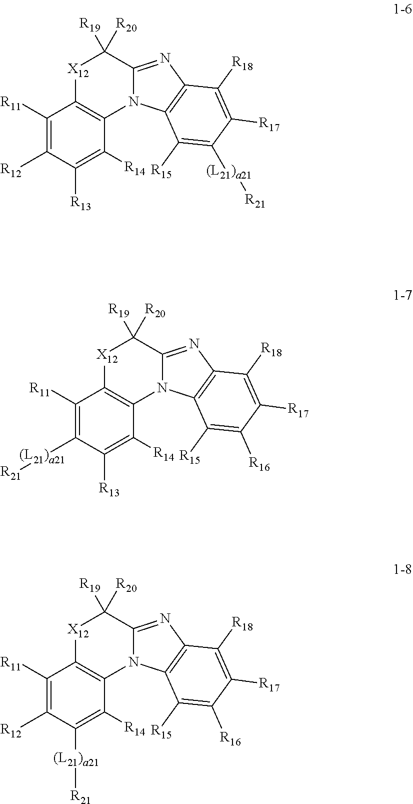
wherein, in Formulae 1-1 to 1-8,
X₁₁ and X₁₂ are each independently O or S, and
L₂₁, a21, and R₁₁ to R₂₁ are the same as described in connection with Formula 1.

20. The heterocyclic compound of claim 9, wherein the heterocyclic compound is of Group I or Group II:
Group I 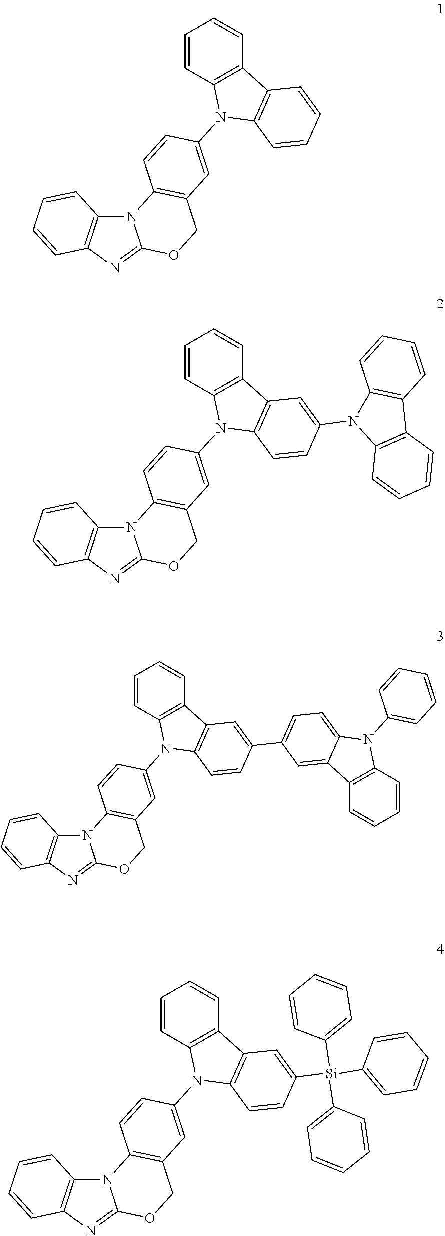 [SMILES: c1ccc2c(c1)nc1n2-c2ccc(-n3c4ccccc4c4ccccc43)cc2CO1 ; c1ccc2c(c1)nc1n2-c2ccc(-n3c4ccccc4c4cc(-n5c6ccccc6c6ccccc65)ccc43)cc2CO1 ; c1ccc(-n2c3ccccc3c3cc(-c4ccc5c(c4)c4ccccc4n5-c4ccc5c(c4)COc4nc6ccccc6n4-5)ccc32)cc1 ; c1ccc([Si](c2ccccc2)(c2ccccc2)c2ccc3c(c2)c2ccccc2n3-c2ccc3c(c2)COc2nc4ccccc4n2-3)cc1] 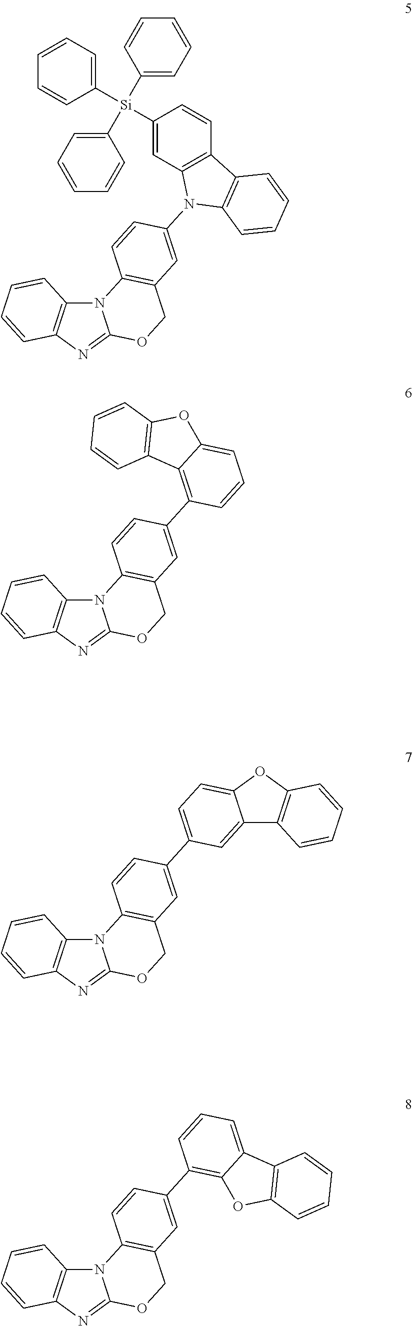 [SMILES: c1ccc([Si](c2ccccc2)(c2ccccc2)c2ccc3c4ccccc4n(-c4ccc5c(c4)COc4nc6ccccc6n4-5)c3c2)cc1 ; c1ccc2c(c1)nc1n2-c2ccc(-c3cccc4oc5ccccc5c34)cc2CO1 ; c1ccc2c(c1)nc1n2-c2ccc(-c3ccc4oc5ccccc5c4c3)cc2CO1 ; c1ccc2c(c1)nc1n2-c2ccc(-c3cccc4c3oc3ccccc34)cc2CO1] 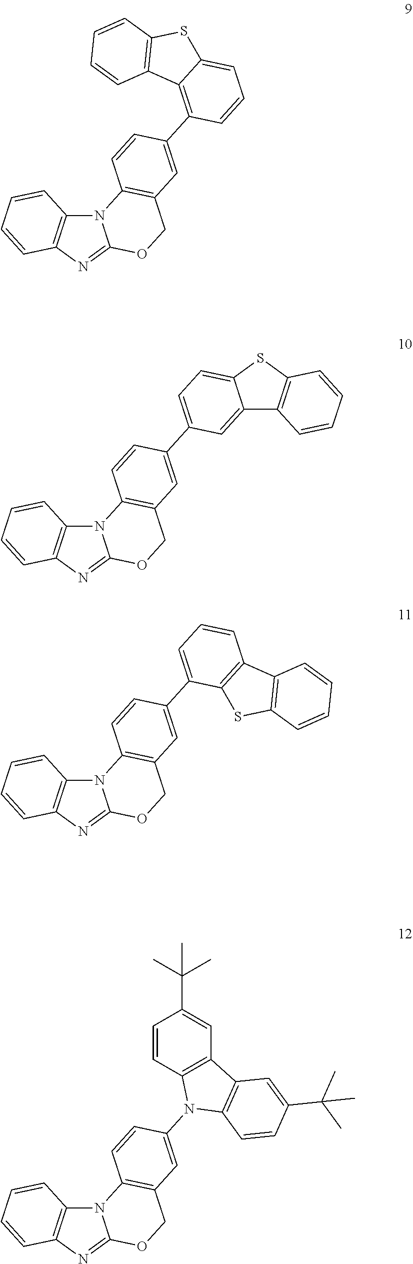 [SMILES: c1ccc2c(c1)nc1n2-c2ccc(-c3cccc4sc5ccccc5c34)cc2CO1 ; c1ccc2c(c1)nc1n2-c2ccc(-c3ccc4sc5ccccc5c4c3)cc2CO1 ; c1ccc2c(c1)nc1n2-c2ccc(-c3cccc4c3sc3ccccc34)cc2CO1 ; CC(C)(C)c1ccc2c(c1)c1cc(C(C)(C)C)ccc1n2-c1ccc2c(c1)COc1nc3ccccc3n1-2] 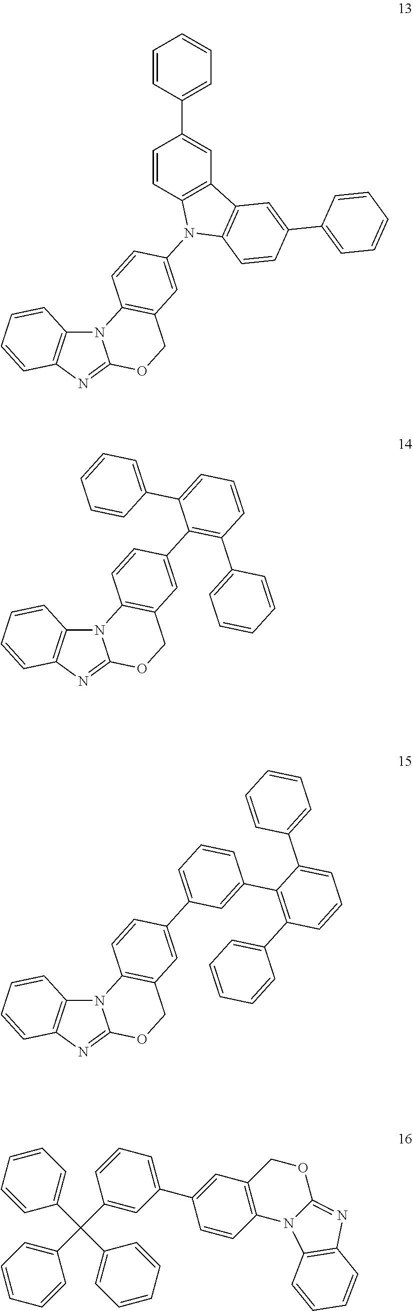 [SMILES: c1ccc(-c2ccc3c(c2)c2cc(-c4ccccc4)ccc2n3-c2ccc3c(c2)COc2nc4ccccc4n2-3)cc1 ; c1ccc(-c2cccc(-c3ccccc3)c2-c2ccc3c(c2)COc2nc4ccccc4n2-3)cc1 ; c1ccc(-c2cccc(-c3ccccc3)c2-c2cccc(-c3ccc4c(c3)COc3nc5ccccc5n3-4)c2)cc1 ; c1ccc(C(c2ccccc2)(c2ccccc2)c2cccc(-c3ccc4c(c3)COc3nc5ccccc5n3-4)c2)cc1] 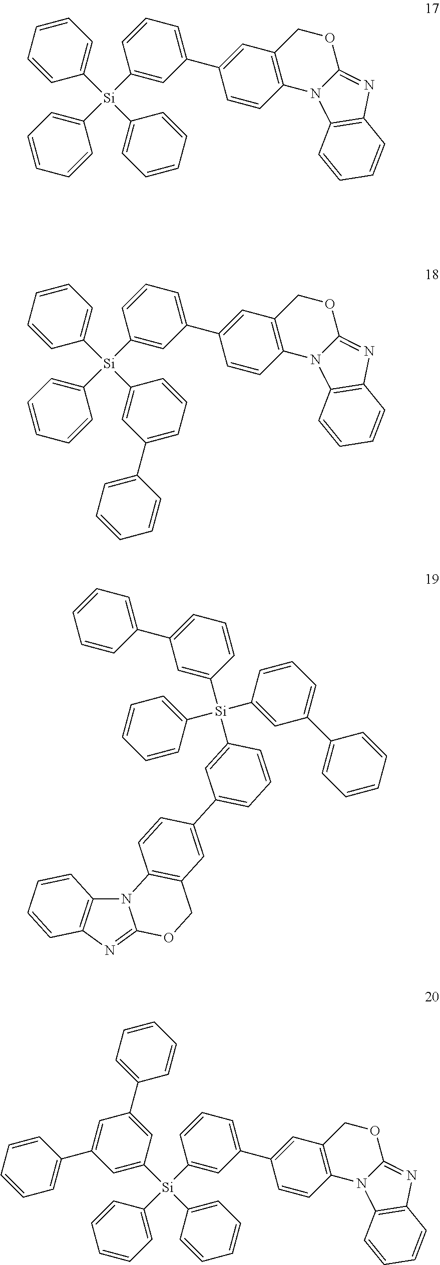 [SMILES: c1ccc([Si](c2ccccc2)(c2ccccc2)c2cccc(-c3ccc4c(c3)COc3nc5ccccc5n3-4)c2)cc1 ; c1ccc(-c2cccc([Si](c3ccccc3)(c3ccccc3)c3cccc(-c4ccc5c(c4)COc4nc6ccccc6n4-5)c3)c2)cc1 ; c1ccc(-c2cccc([Si](c3ccccc3)(c3cccc(-c4ccccc4)c3)c3cccc(-c4ccc5c(c4)COc4nc6ccccc6n4-5)c3)c2)cc1 ; c1ccc(-c2cc(-c3ccccc3)cc([Si](c3ccccc3)(c3ccccc3)c3cccc(-c4ccc5c(c4)COc4nc6ccccc6n4-5)c3)c2)cc1] 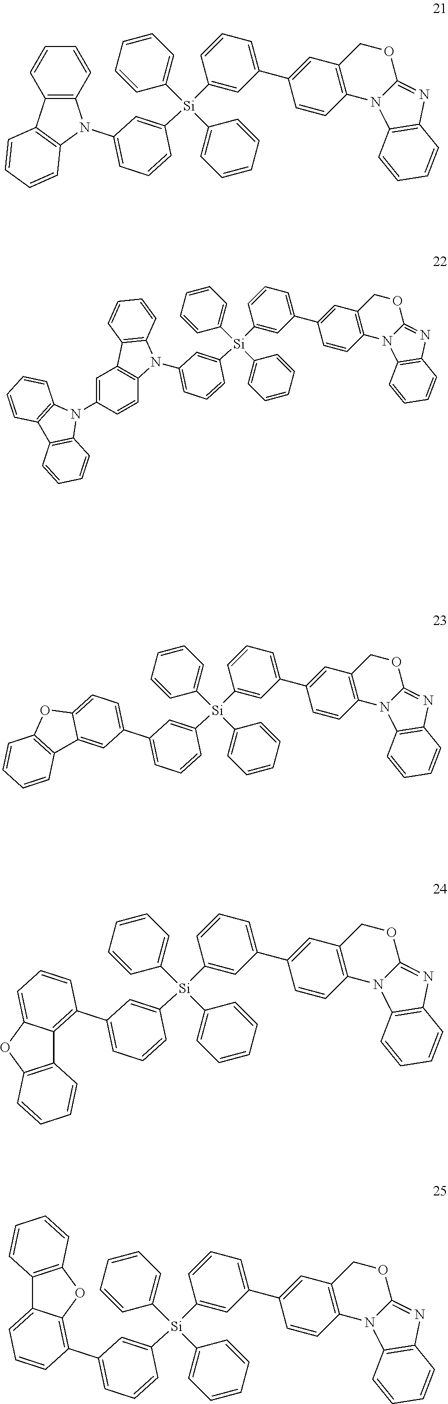 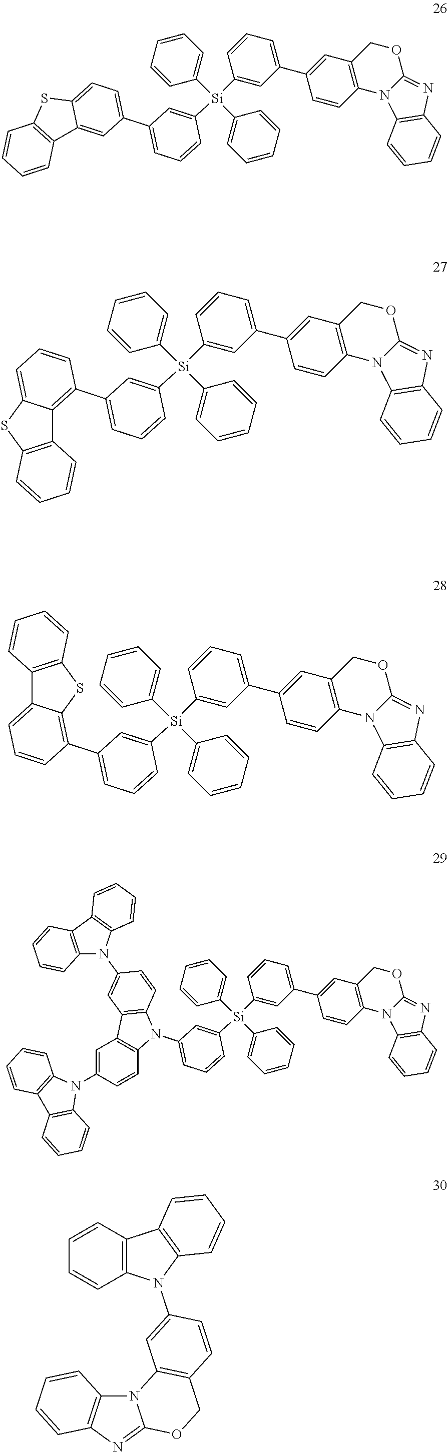 [SMILES: c1ccc([Si](c2ccccc2)(c2cccc(-c3ccc4c(c3)COc3nc5ccccc5n3-4)c2)c2cccc(-c3ccc4sc5ccccc5c4c3)c2)cc1 ; c1ccc([Si](c2ccccc2)(c2cccc(-c3ccc4c(c3)COc3nc5ccccc5n3-4)c2)c2cccc(-c3cccc4sc5ccccc5c34)c2)cc1 ; c1ccc([Si](c2ccccc2)(c2cccc(-c3ccc4c(c3)COc3nc5ccccc5n3-4)c2)c2cccc(-c3cccc4c3sc3ccccc34)c2)cc1 ; c1ccc([Si](c2ccccc2)(c2cccc(-c3ccc4c(c3)COc3nc5ccccc5n3-4)c2)c2cccc(-n3c4ccc(-n5c6ccccc6c6ccccc65)cc4c4cc(-n5c6ccccc6c6ccccc65)ccc43)c2)cc1 ; c1ccc2c(c1)nc1n2-c2cc(-n3c4ccccc4c4ccccc43)ccc2CO1] 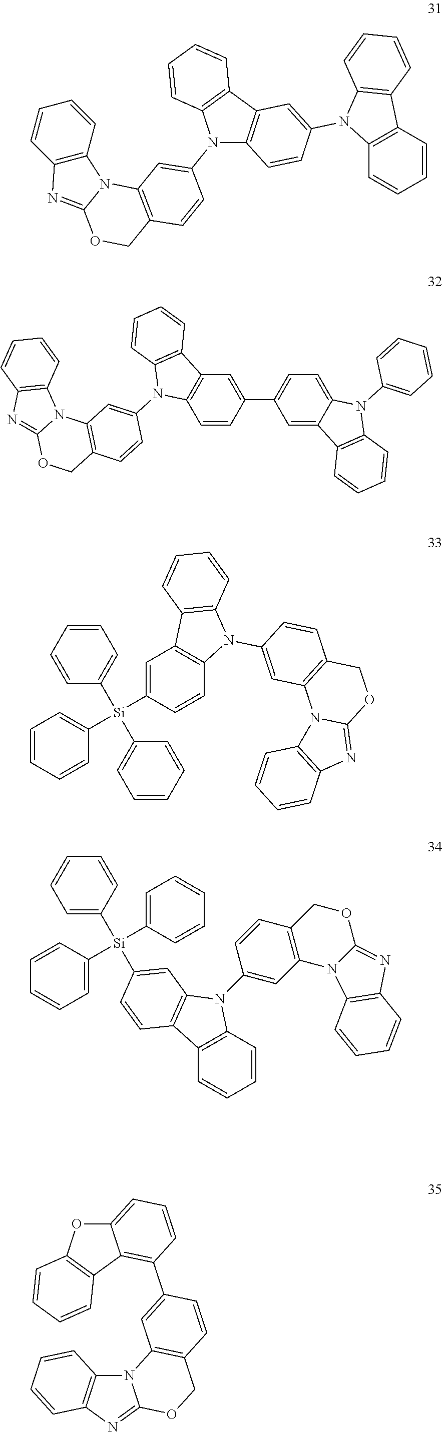 [SMILES: c1ccc2c(c1)nc1n2-c2cc(-n3c4ccccc4c4cc(-n5c6ccccc6c6ccccc65)ccc43)ccc2CO1 ; c1ccc(-n2c3ccccc3c3cc(-c4ccc5c(c4)c4ccccc4n5-c4ccc5c(c4)-n4c(nc6ccccc64)OC5)ccc32)cc1 ; c1ccc([Si](c2ccccc2)(c2ccccc2)c2ccc3c(c2)c2ccccc2n3-c2ccc3c(c2)-n2c(nc4ccccc42)OC3)cc1 ; c1ccc([Si](c2ccccc2)(c2ccccc2)c2ccc3c4ccccc4n(-c4ccc5c(c4)-n4c(nc6ccccc64)OC5)c3c2)cc1 ; c1ccc2c(c1)nc1n2-c2cc(-c3cccc4oc5ccccc5c34)ccc2CO1] 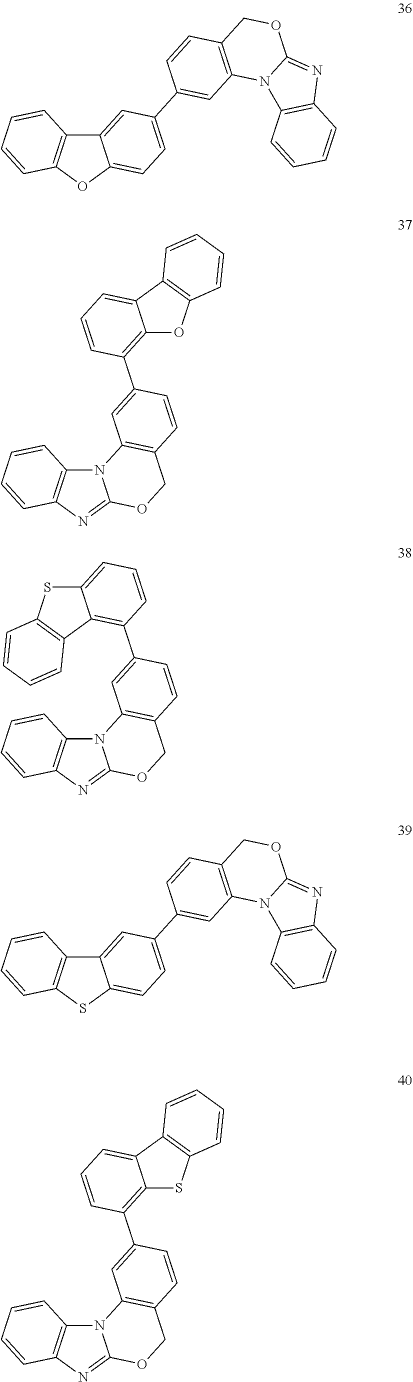 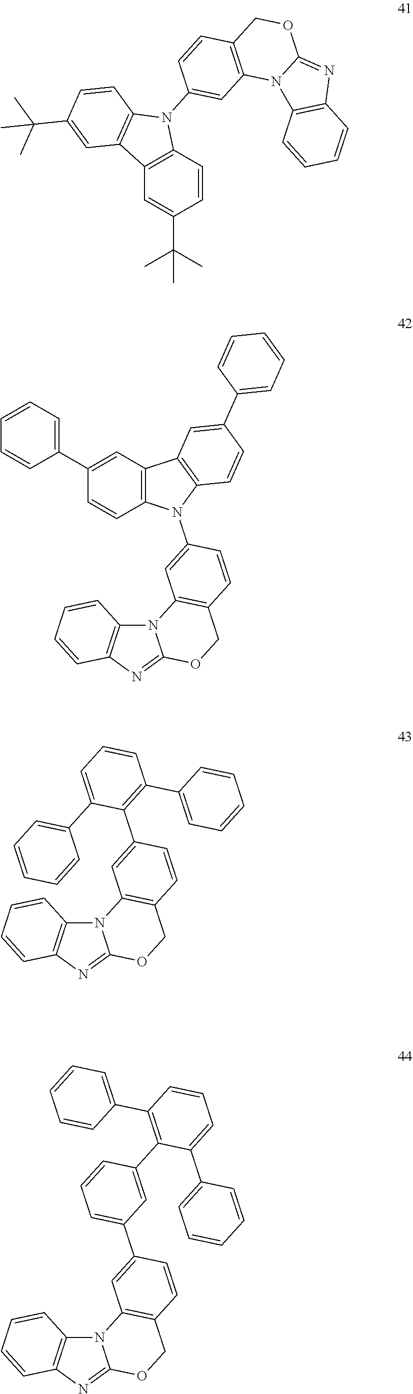 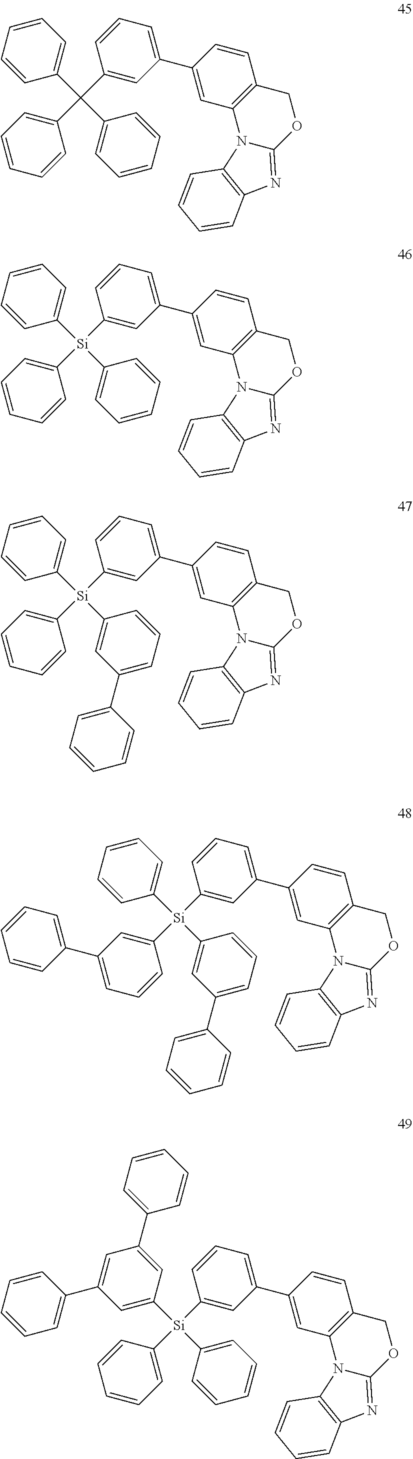 [SMILES: c1ccc(C(c2ccccc2)(c2ccccc2)c2cccc(-c3ccc4c(c3)-n3c(nc5ccccc53)OC4)c2)cc1 ; c1ccc([Si](c2ccccc2)(c2ccccc2)c2cccc(-c3ccc4c(c3)-n3c(nc5ccccc53)OC4)c2)cc1 ; c1ccc(-c2cccc([Si](c3ccccc3)(c3ccccc3)c3cccc(-c4ccc5c(c4)-n4c(nc6ccccc64)OC5)c3)c2)cc1 ; c1ccc(-c2cccc([Si](c3ccccc3)(c3cccc(-c4ccccc4)c3)c3cccc(-c4ccc5c(c4)-n4c(nc6ccccc64)OC5)c3)c2)cc1 ; c1ccc(-c2cc(-c3ccccc3)cc([Si](c3ccccc3)(c3ccccc3)c3cccc(-c4ccc5c(c4)-n4c(nc6ccccc64)OC5)c3)c2)cc1] 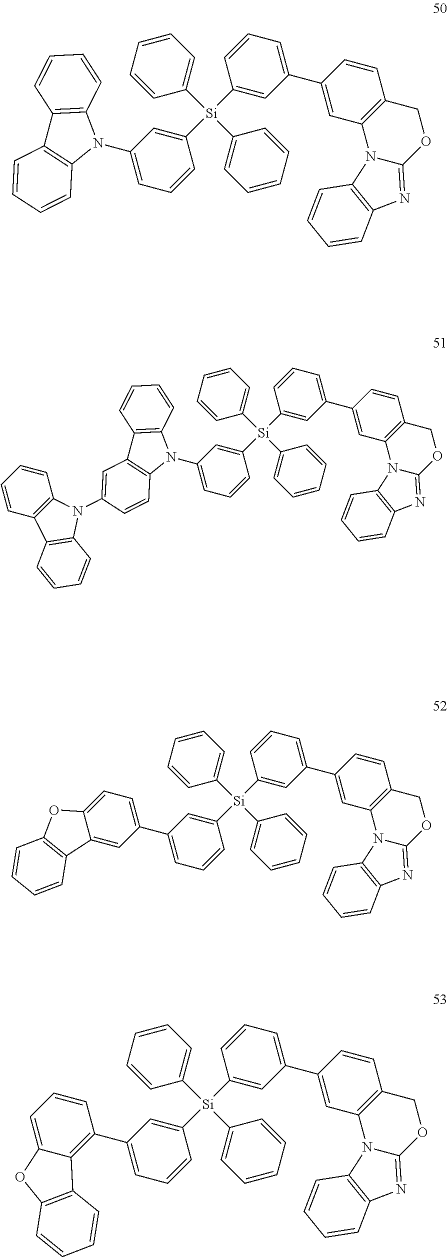 [SMILES: c1ccc([Si](c2ccccc2)(c2cccc(-c3ccc4c(c3)-n3c(nc5ccccc53)OC4)c2)c2cccc(-n3c4ccccc4c4ccccc43)c2)cc1 ; c1ccc([Si](c2ccccc2)(c2cccc(-c3ccc4c(c3)-n3c(nc5ccccc53)OC4)c2)c2cccc(-n3c4ccccc4c4cc(-n5c6ccccc6c6ccccc65)ccc43)c2)cc1 ; c1ccc([Si](c2ccccc2)(c2cccc(-c3ccc4c(c3)-n3c(nc5ccccc53)OC4)c2)c2cccc(-c3ccc4oc5ccccc5c4c3)c2)cc1 ; c1ccc([Si](c2ccccc2)(c2cccc(-c3ccc4c(c3)-n3c(nc5ccccc53)OC4)c2)c2cccc(-c3cccc4oc5ccccc5c34)c2)cc1] 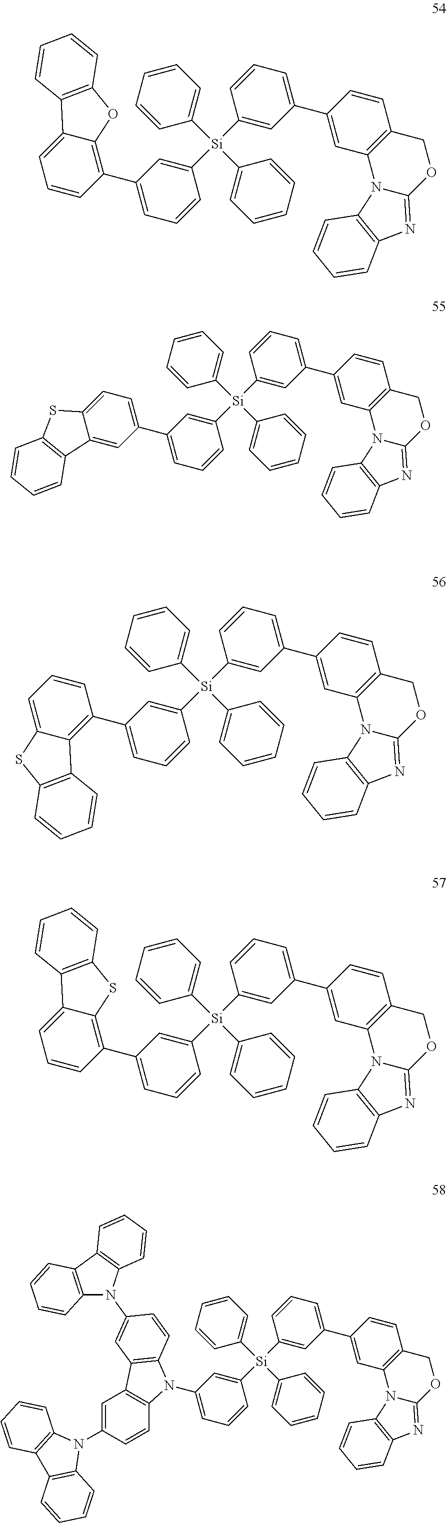 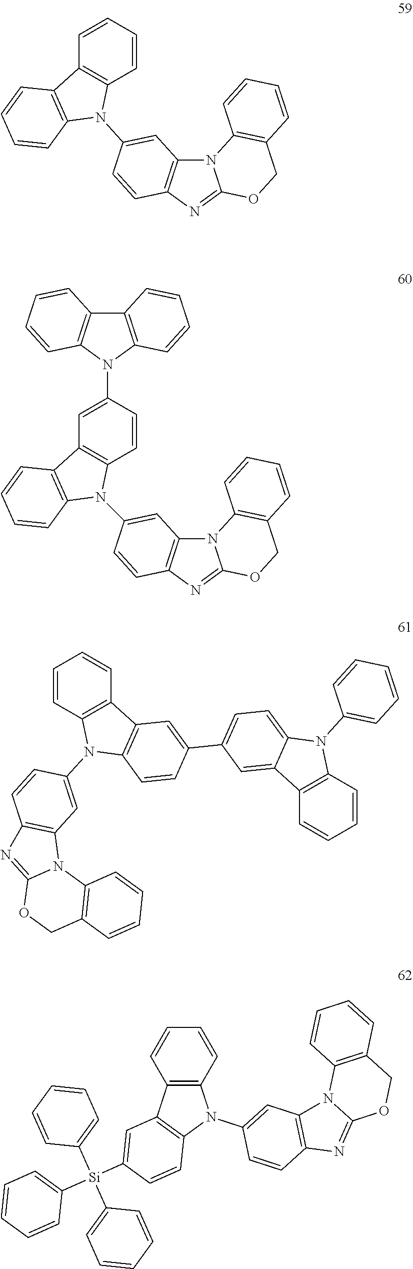 [SMILES: c1ccc2c(c1)COc1nc3ccc(-n4c5ccccc5c5ccccc54)cc3n1-2 ; c1ccc2c(c1)COc1nc3ccc(-n4c5ccccc5c5cc(-n6c7ccccc7c7ccccc76)ccc54)cc3n1-2 ; c1ccc(-n2c3ccccc3c3cc(-c4ccc5c(c4)c4ccccc4n5-c4ccc5nc6n(c5c4)-c4ccccc4CO6)ccc32)cc1 ; c1ccc([Si](c2ccccc2)(c2ccccc2)c2ccc3c(c2)c2ccccc2n3-c2ccc3nc4n(c3c2)-c2ccccc2CO4)cc1] 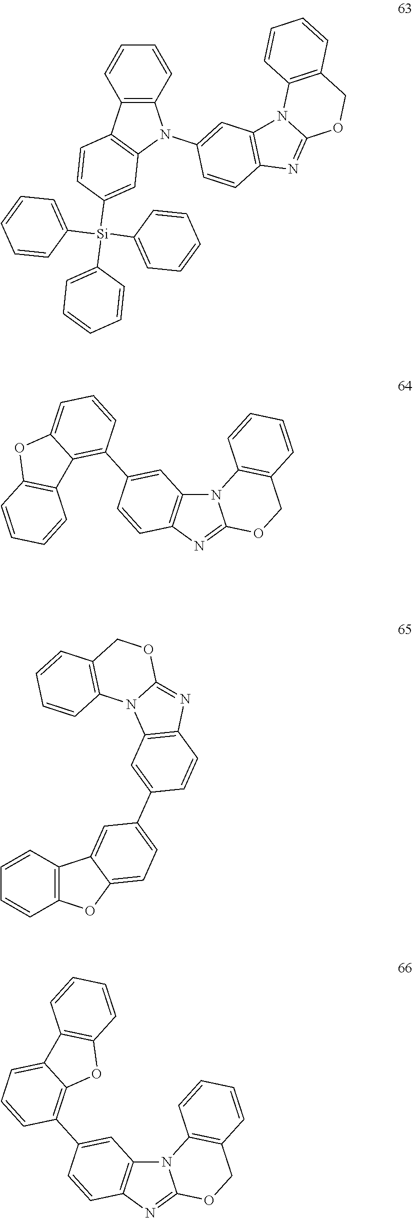 [SMILES: c1ccc([Si](c2ccccc2)(c2ccccc2)c2ccc3c4ccccc4n(-c4ccc5nc6n(c5c4)-c4ccccc4CO6)c3c2)cc1 ; c1ccc2c(c1)COc1nc3ccc(-c4cccc5oc6ccccc6c45)cc3n1-2 ; c1ccc2c(c1)COc1nc3ccc(-c4ccc5oc6ccccc6c5c4)cc3n1-2 ; c1ccc2c(c1)COc1nc3ccc(-c4cccc5c4oc4ccccc45)cc3n1-2] 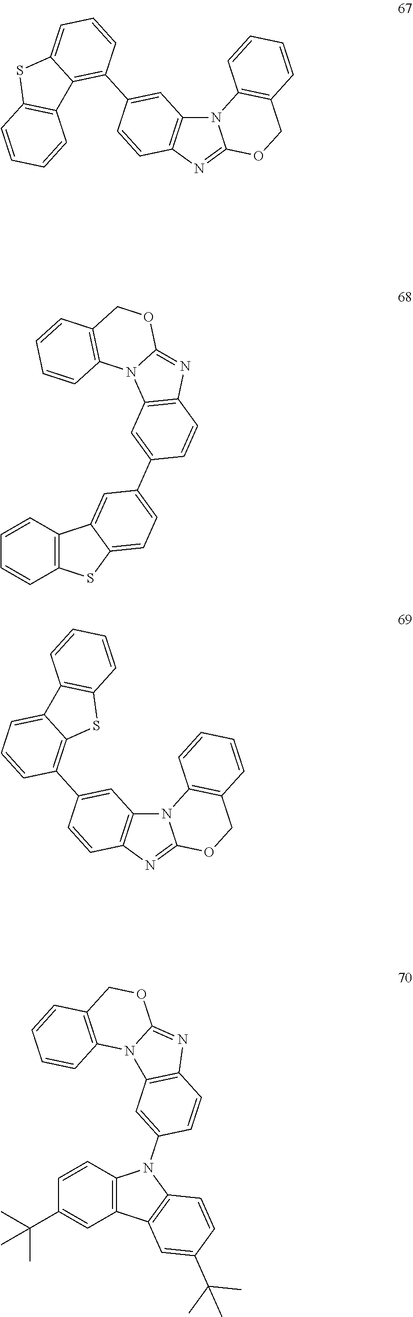 [SMILES: c1ccc2c(c1)COc1nc3ccc(-c4cccc5sc6ccccc6c45)cc3n1-2 ; c1ccc2c(c1)COc1nc3ccc(-c4ccc5sc6ccccc6c5c4)cc3n1-2 ; c1ccc2c(c1)COc1nc3ccc(-c4cccc5c4sc4ccccc45)cc3n1-2 ; CC(C)(C)c1ccc2c(c1)c1cc(C(C)(C)C)ccc1n2-c1ccc2nc3n(c2c1)-c1ccccc1CO3] 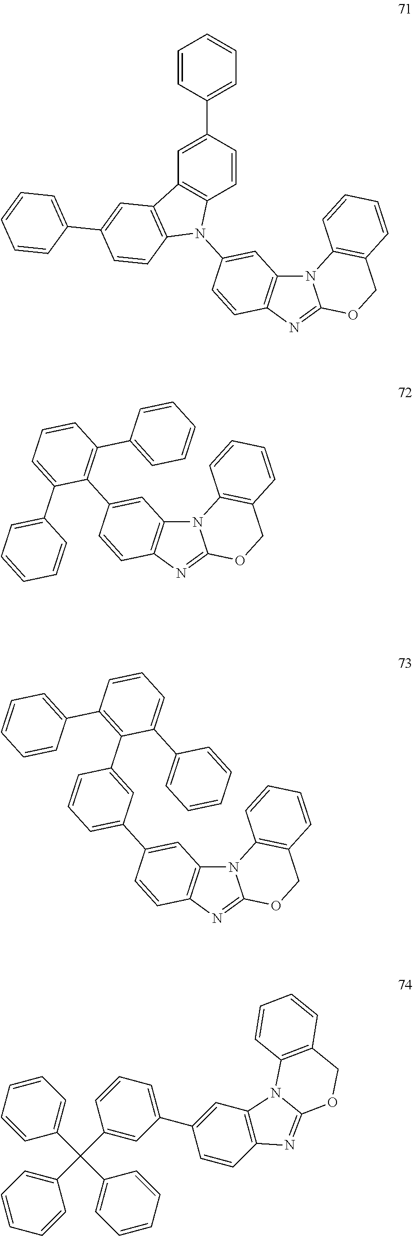 [SMILES: c1ccc(-c2ccc3c(c2)c2cc(-c4ccccc4)ccc2n3-c2ccc3nc4n(c3c2)-c2ccccc2CO4)cc1 ; c1ccc(-c2cccc(-c3ccccc3)c2-c2ccc3nc4n(c3c2)-c2ccccc2CO4)cc1 ; c1ccc(-c2cccc(-c3ccccc3)c2-c2cccc(-c3ccc4nc5n(c4c3)-c3ccccc3CO5)c2)cc1 ; c1ccc(C(c2ccccc2)(c2ccccc2)c2cccc(-c3ccc4nc5n(c4c3)-c3ccccc3CO5)c2)cc1] 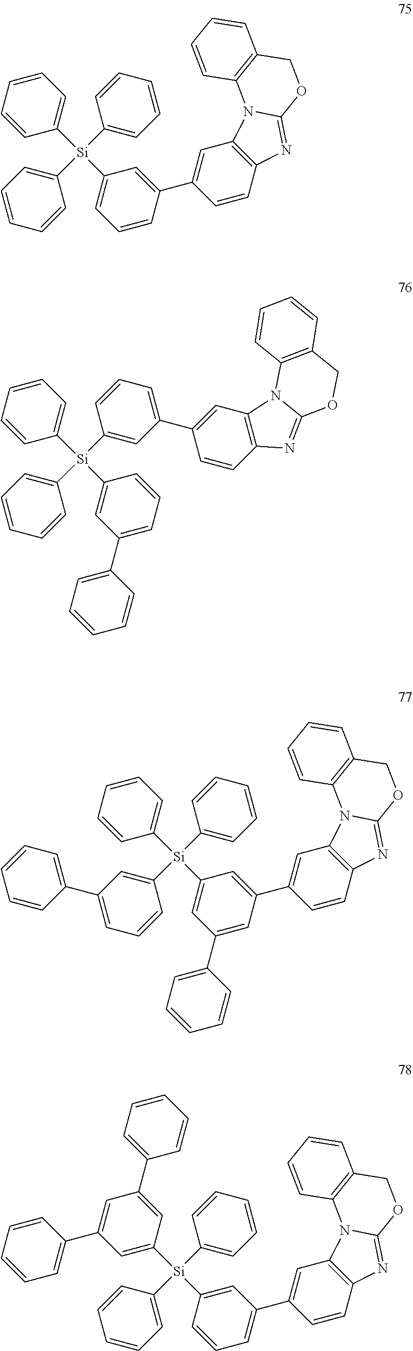 [SMILES: c1ccc([Si](c2ccccc2)(c2ccccc2)c2cccc(-c3ccc4nc5n(c4c3)-c3ccccc3CO5)c2)cc1 ; c1ccc(-c2cccc([Si](c3ccccc3)(c3ccccc3)c3cccc(-c4ccc5nc6n(c5c4)-c4ccccc4CO6)c3)c2)cc1 ; c1ccc(-c2cccc([Si](c3ccccc3)(c3ccccc3)c3cc(-c4ccccc4)cc(-c4ccc5nc6n(c5c4)-c4ccccc4CO6)c3)c2)cc1 ; c1ccc(-c2cc(-c3ccccc3)cc([Si](c3ccccc3)(c3ccccc3)c3cccc(-c4ccc5nc6n(c5c4)-c4ccccc4CO6)c3)c2)cc1] 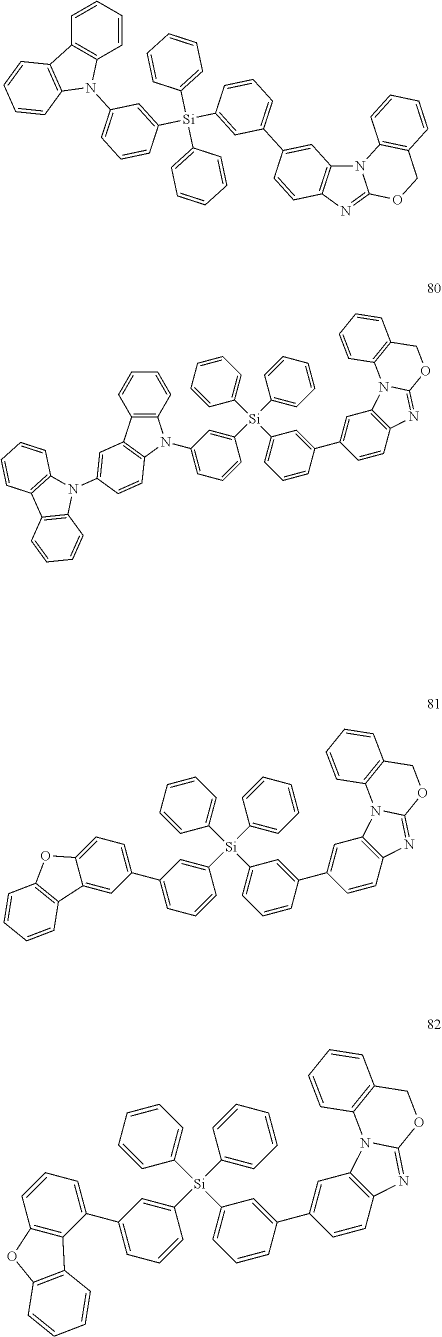 [SMILES: c1ccc([Si](c2ccccc2)(c2cccc(-c3ccc4nc5n(c4c3)-c3ccccc3CO5)c2)c2cccc(-n3c4ccccc4c4ccccc43)c2)cc1 ; c1ccc([Si](c2ccccc2)(c2cccc(-c3ccc4nc5n(c4c3)-c3ccccc3CO5)c2)c2cccc(-n3c4ccccc4c4cc(-n5c6ccccc6c6ccccc65)ccc43)c2)cc1 ; c1ccc([Si](c2ccccc2)(c2cccc(-c3ccc4oc5ccccc5c4c3)c2)c2cccc(-c3ccc4nc5n(c4c3)-c3ccccc3CO5)c2)cc1 ; c1ccc([Si](c2ccccc2)(c2cccc(-c3ccc4nc5n(c4c3)-c3ccccc3CO5)c2)c2cccc(-c3cccc4oc5ccccc5c34)c2)cc1] 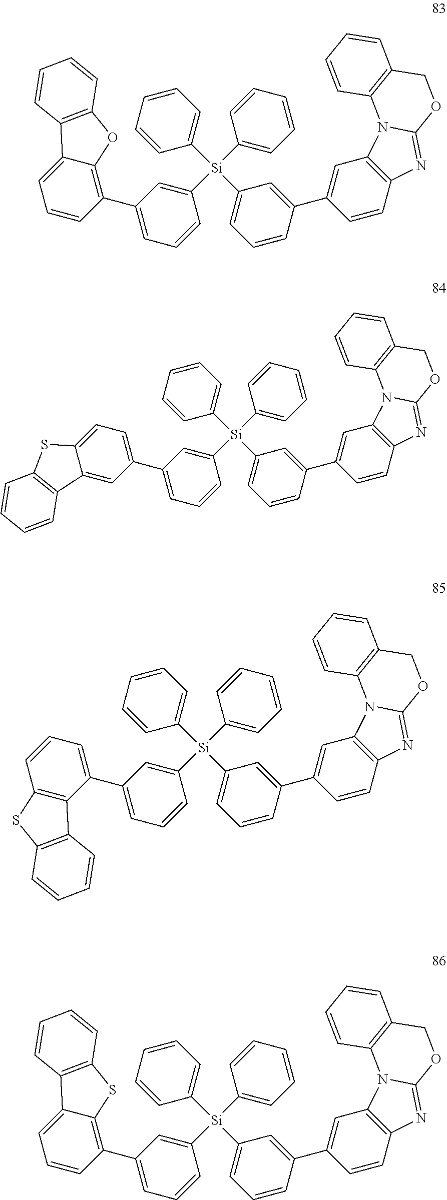 [SMILES: c1ccc([Si](c2ccccc2)(c2cccc(-c3ccc4nc5n(c4c3)-c3ccccc3CO5)c2)c2cccc(-c3cccc4c3oc3ccccc34)c2)cc1 ; c1ccc([Si](c2ccccc2)(c2cccc(-c3ccc4sc5ccccc5c4c3)c2)c2cccc(-c3ccc4nc5n(c4c3)-c3ccccc3CO5)c2)cc1 ; c1ccc([Si](c2ccccc2)(c2cccc(-c3ccc4nc5n(c4c3)-c3ccccc3CO5)c2)c2cccc(-c3cccc4sc5ccccc5c34)c2)cc1 ; c1ccc([Si](c2ccccc2)(c2cccc(-c3ccc4nc5n(c4c3)-c3ccccc3CO5)c2)c2cccc(-c3cccc4c3sc3ccccc34)c2)cc1] 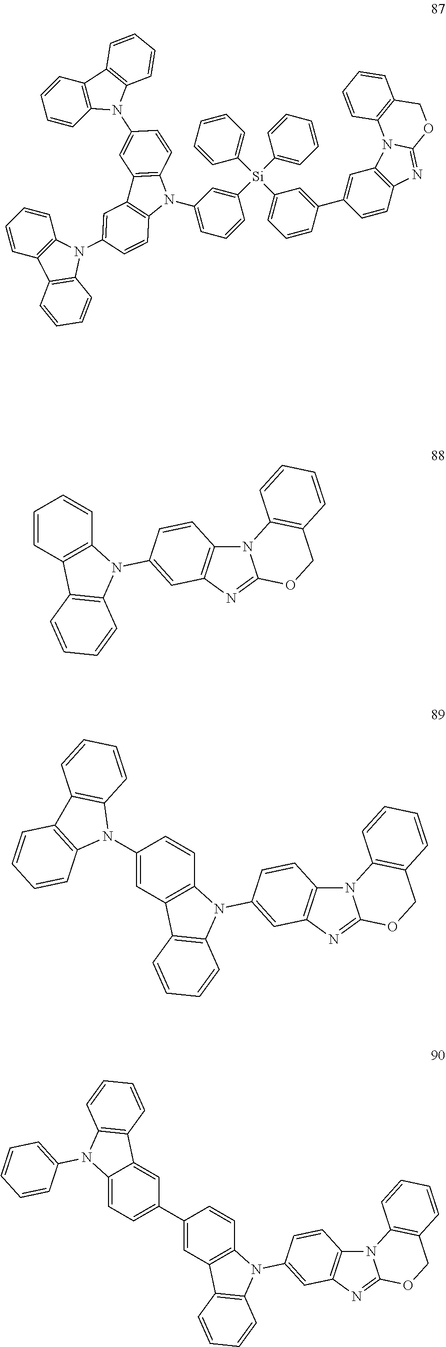 [SMILES: c1ccc([Si](c2ccccc2)(c2cccc(-c3ccc4nc5n(c4c3)-c3ccccc3CO5)c2)c2cccc(-n3c4ccc(-n5c6ccccc6c6ccccc65)cc4c4cc(-n5c6ccccc6c6ccccc65)ccc43)c2)cc1 ; c1ccc2c(c1)COc1nc3cc(-n4c5ccccc5c5ccccc54)ccc3n1-2 ; c1ccc2c(c1)COc1nc3cc(-n4c5ccccc5c5cc(-n6c7ccccc7c7ccccc76)ccc54)ccc3n1-2 ; c1ccc(-n2c3ccccc3c3cc(-c4ccc5c(c4)c4ccccc4n5-c4ccc5c(c4)nc4n5-c5ccccc5CO4)ccc32)cc1] 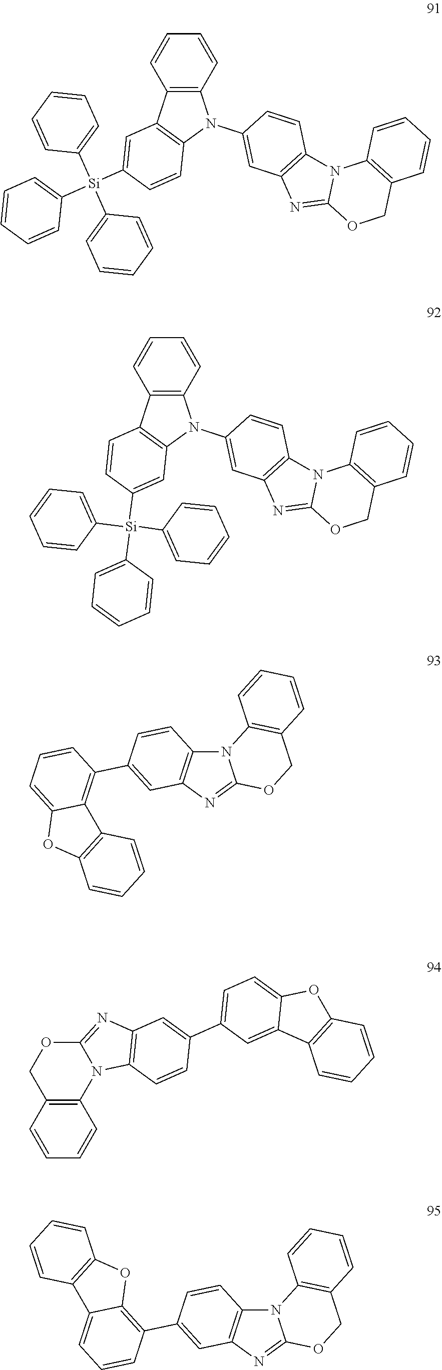 [SMILES: c1ccc([Si](c2ccccc2)(c2ccccc2)c2ccc3c(c2)c2ccccc2n3-c2ccc3c(c2)nc2n3-c3ccccc3CO2)cc1 ; c1ccc([Si](c2ccccc2)(c2ccccc2)c2ccc3c4ccccc4n(-c4ccc5c(c4)nc4n5-c5ccccc5CO4)c3c2)cc1 ; c1ccc2c(c1)COc1nc3cc(-c4cccc5oc6ccccc6c45)ccc3n1-2 ; c1ccc2c(c1)COc1nc3cc(-c4ccc5oc6ccccc6c5c4)ccc3n1-2 ; c1ccc2c(c1)COc1nc3cc(-c4cccc5c4oc4ccccc45)ccc3n1-2] 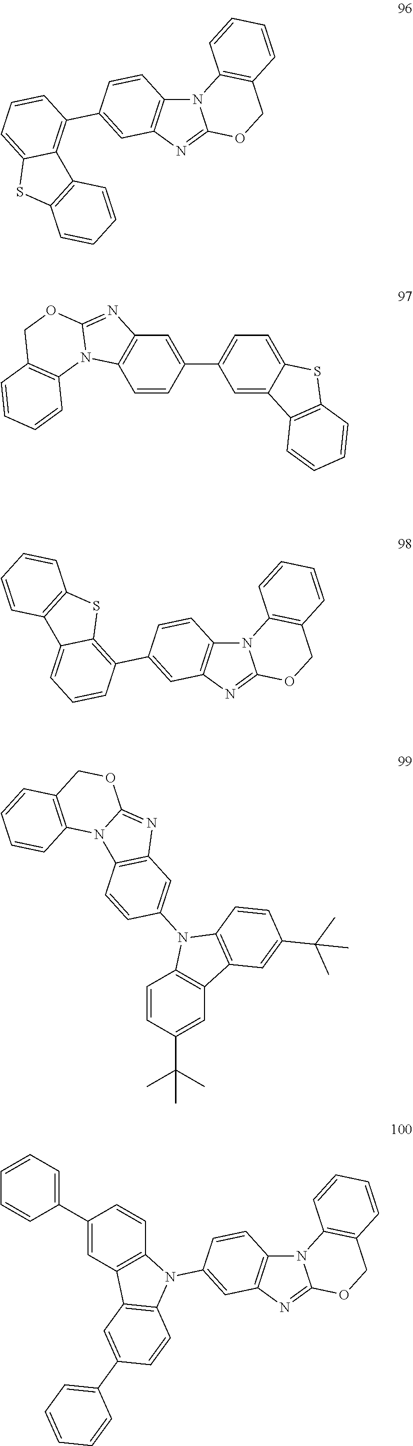 [SMILES: c1ccc2c(c1)COc1nc3cc(-c4cccc5sc6ccccc6c45)ccc3n1-2 ; c1ccc2c(c1)COc1nc3cc(-c4ccc5sc6ccccc6c5c4)ccc3n1-2 ; c1ccc2c(c1)COc1nc3cc(-c4cccc5c4sc4ccccc45)ccc3n1-2 ; CC(C)(C)c1ccc2c(c1)c1cc(C(C)(C)C)ccc1n2-c1ccc2c(c1)nc1n2-c2ccccc2CO1 ; c1ccc(-c2ccc3c(c2)c2cc(-c4ccccc4)ccc2n3-c2ccc3c(c2)nc2n3-c3ccccc3CO2)cc1] 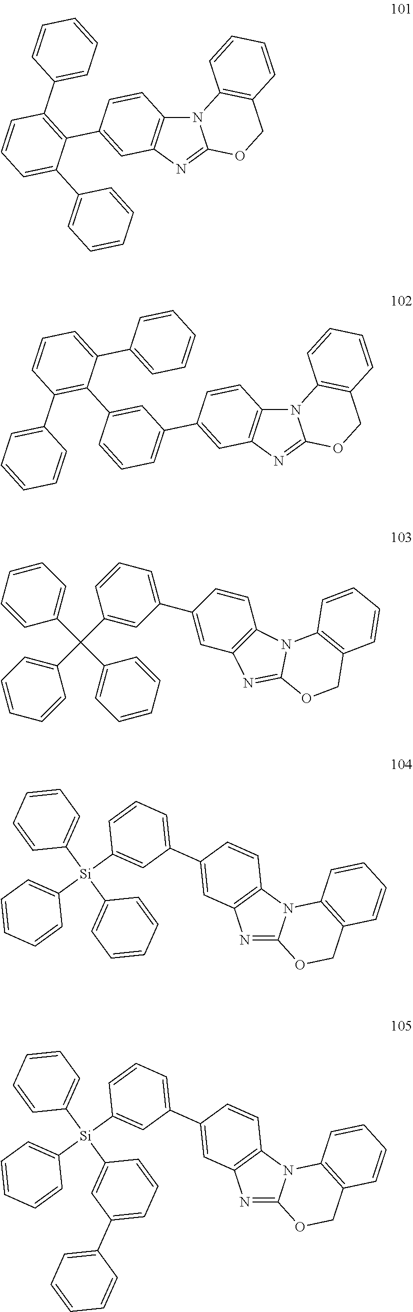 [SMILES: c1ccc(-c2cccc(-c3ccccc3)c2-c2ccc3c(c2)nc2n3-c3ccccc3CO2)cc1 ; c1ccc(-c2cccc(-c3ccccc3)c2-c2cccc(-c3ccc4c(c3)nc3n4-c4ccccc4CO3)c2)cc1 ; c1ccc(C(c2ccccc2)(c2ccccc2)c2cccc(-c3ccc4c(c3)nc3n4-c4ccccc4CO3)c2)cc1 ; c1ccc([Si](c2ccccc2)(c2ccccc2)c2cccc(-c3ccc4c(c3)nc3n4-c4ccccc4CO3)c2)cc1 ; c1ccc(-c2cccc([Si](c3ccccc3)(c3ccccc3)c3cccc(-c4ccc5c(c4)nc4n5-c5ccccc5CO4)c3)c2)cc1] 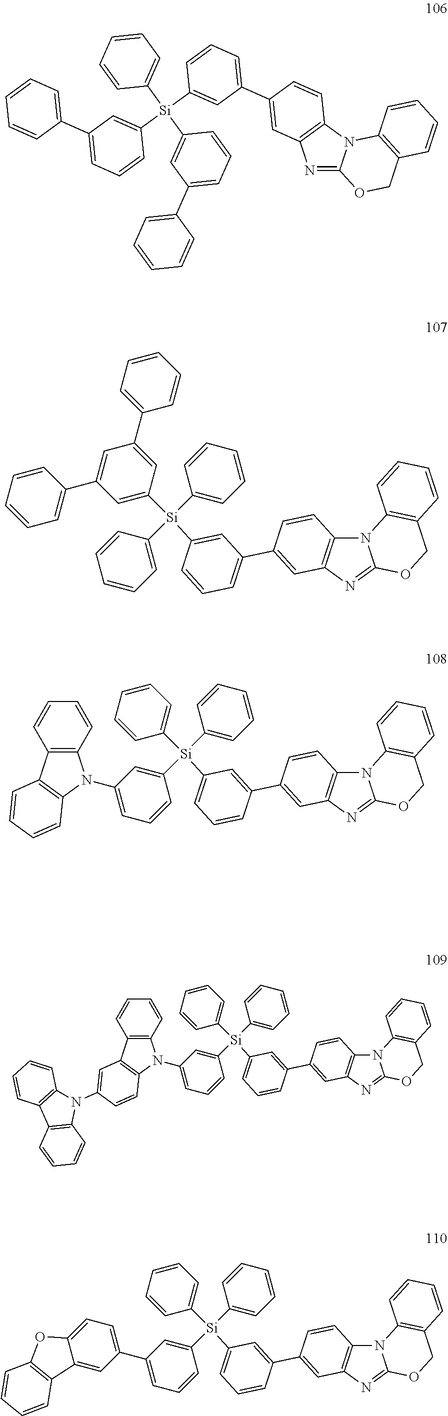 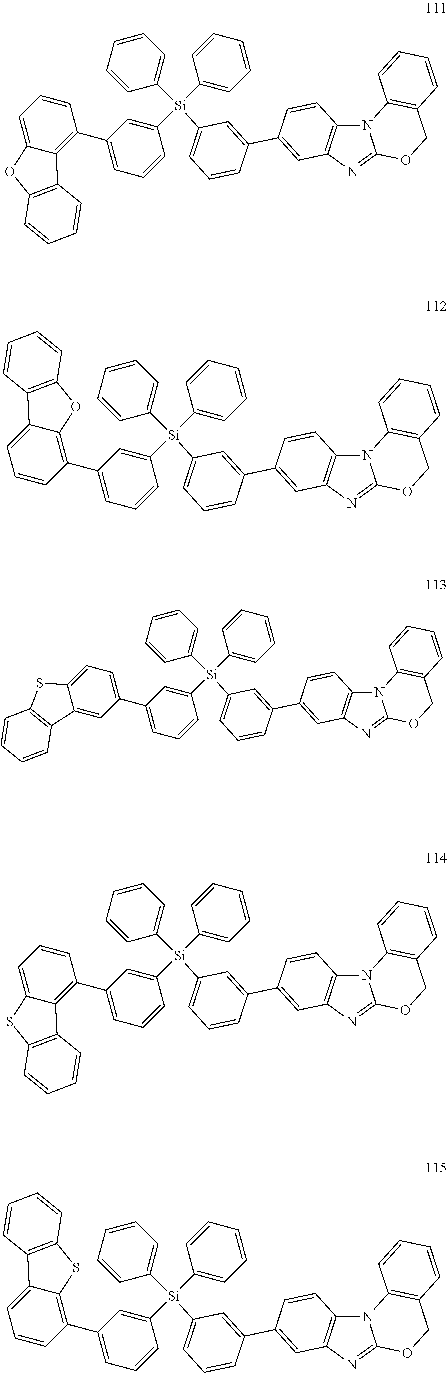 [SMILES: c1ccc([Si](c2ccccc2)(c2cccc(-c3ccc4c(c3)nc3n4-c4ccccc4CO3)c2)c2cccc(-c3cccc4oc5ccccc5c34)c2)cc1 ; c1ccc([Si](c2ccccc2)(c2cccc(-c3ccc4c(c3)nc3n4-c4ccccc4CO3)c2)c2cccc(-c3cccc4c3oc3ccccc34)c2)cc1 ; c1ccc([Si](c2ccccc2)(c2cccc(-c3ccc4c(c3)nc3n4-c4ccccc4CO3)c2)c2cccc(-c3ccc4sc5ccccc5c4c3)c2)cc1 ; c1ccc([Si](c2ccccc2)(c2cccc(-c3ccc4c(c3)nc3n4-c4ccccc4CO3)c2)c2cccc(-c3cccc4sc5ccccc5c34)c2)cc1 ; c1ccc([Si](c2ccccc2)(c2cccc(-c3ccc4c(c3)nc3n4-c4ccccc4CO3)c2)c2cccc(-c3cccc4c3sc3ccccc34)c2)cc1] 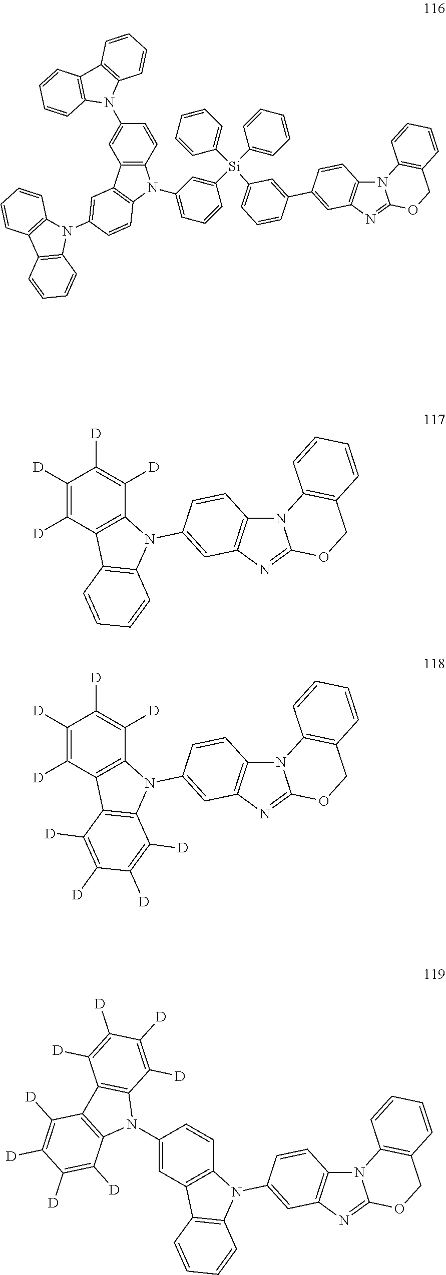 [SMILES: c1ccc([Si](c2ccccc2)(c2cccc(-c3ccc4c(c3)nc3n4-c4ccccc4CO3)c2)c2cccc(-n3c4ccc(-n5c6ccccc6c6ccccc65)cc4c4cc(-n5c6ccccc6c6ccccc65)ccc43)c2)cc1 ; [2H]c1c([2H])c([2H])c2c(c1[2H])c1ccccc1n2-c1ccc2c(c1)nc1n2-c2ccccc2CO1 ; [2H]c1c([2H])c([2H])c2c(c1[2H])c1c([2H])c([2H])c([2H])c([2H])c1n2-c1ccc2c(c1)nc1n2-c2ccccc2CO1 ; [2H]c1c([2H])c([2H])c2c(c1[2H])c1c([2H])c([2H])c([2H])c([2H])c1n2-c1ccc2c(c1)c1ccccc1n2-c1ccc2c(c1)nc1n2-c2ccccc2CO1] 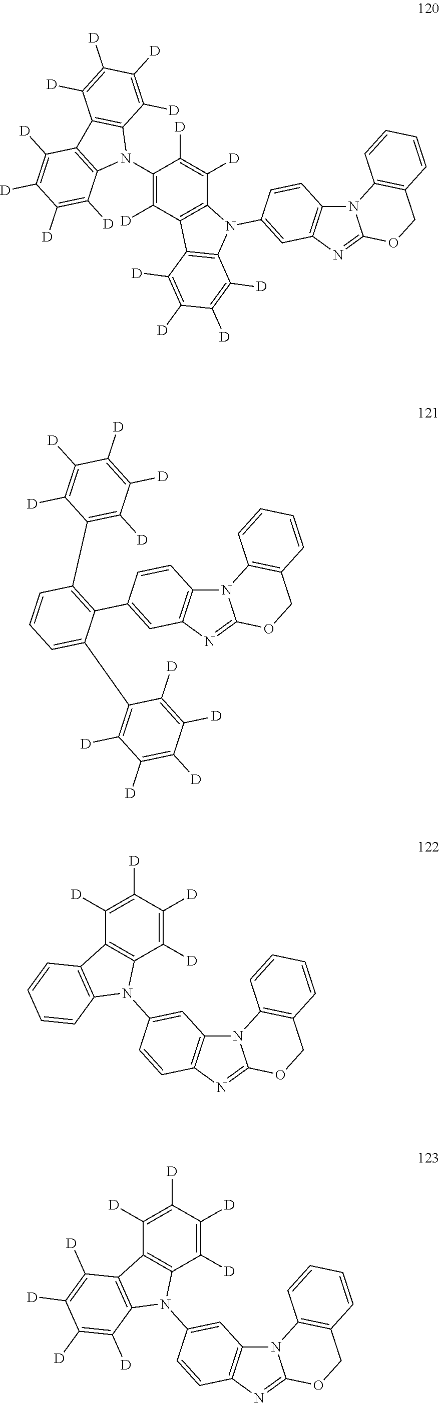 [SMILES: [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc2c(c1)nc1n2-c2ccccc2CO1 ; [2H]c1c([2H])c([2H])c(-c2cccc(-c3c([2H])c([2H])c([2H])c([2H])c3[2H])c2-c2ccc3c(c2)nc2n3-c3ccccc3CO2)c([2H])c1[2H] ; [2H]c1c([2H])c([2H])c2c(c1[2H])c1ccccc1n2-c1ccc2nc3n(c2c1)-c1ccccc1CO3 ; [2H]c1c([2H])c([2H])c2c(c1[2H])c1c([2H])c([2H])c([2H])c([2H])c1n2-c1ccc2nc3n(c2c1)-c1ccccc1CO3] 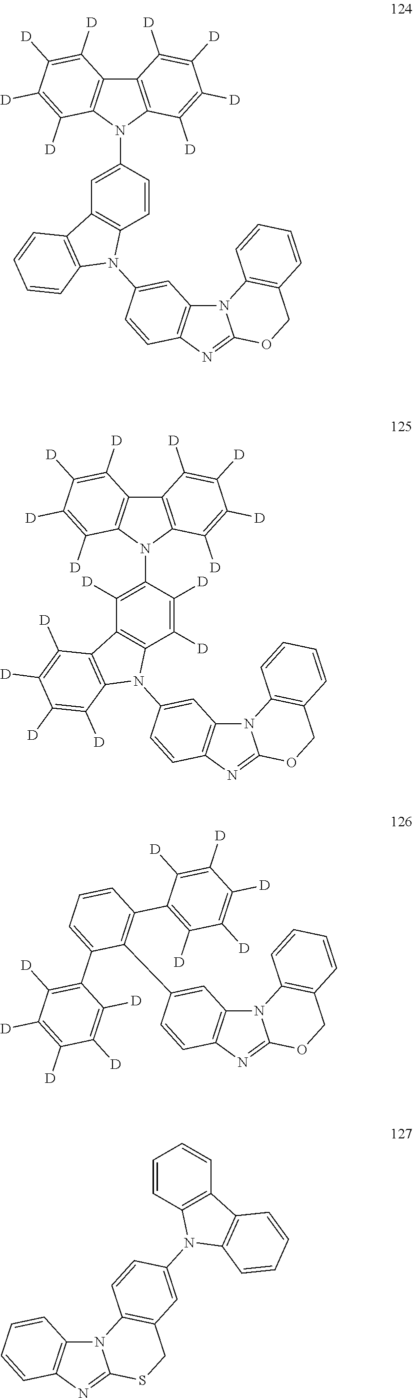 [SMILES: [2H]c1c([2H])c([2H])c2c(c1[2H])c1c([2H])c([2H])c([2H])c([2H])c1n2-c1ccc2c(c1)c1ccccc1n2-c1ccc2nc3n(c2c1)-c1ccccc1CO3 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc2nc3n(c2c1)-c1ccccc1CO3 ; [2H]c1c([2H])c([2H])c(-c2cccc(-c3c([2H])c([2H])c([2H])c([2H])c3[2H])c2-c2ccc3nc4n(c3c2)-c2ccccc2CO4)c([2H])c1[2H] ; c1ccc2c(c1)nc1n2-c2ccc(-n3c4ccccc4c4ccccc43)cc2CS1] 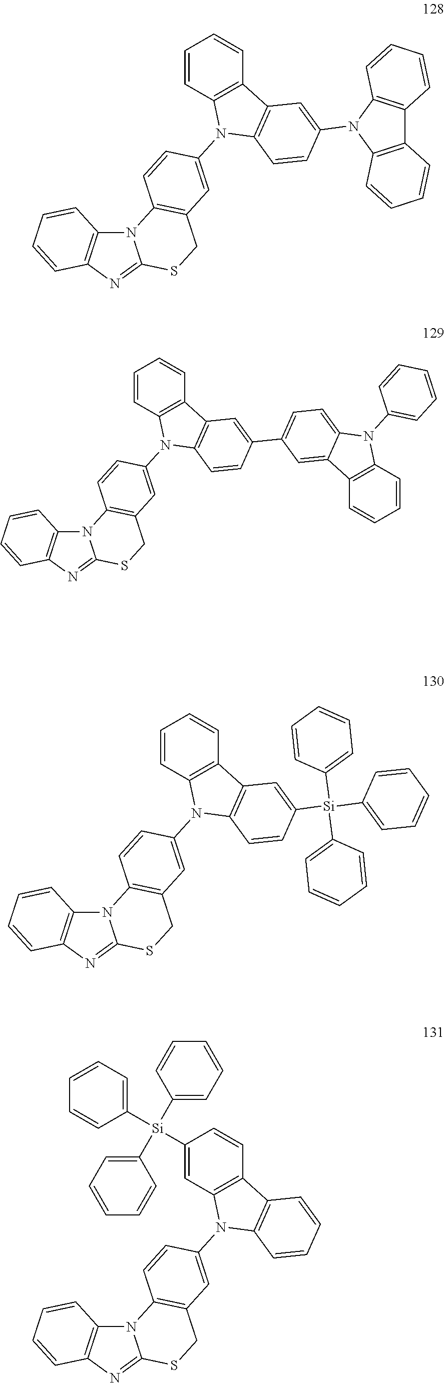 [SMILES: c1ccc2c(c1)nc1n2-c2ccc(-n3c4ccccc4c4cc(-n5c6ccccc6c6ccccc65)ccc43)cc2CS1 ; c1ccc(-n2c3ccccc3c3cc(-c4ccc5c(c4)c4ccccc4n5-c4ccc5c(c4)CSc4nc6ccccc6n4-5)ccc32)cc1 ; c1ccc([Si](c2ccccc2)(c2ccccc2)c2ccc3c(c2)c2ccccc2n3-c2ccc3c(c2)CSc2nc4ccccc4n2-3)cc1 ; c1ccc([Si](c2ccccc2)(c2ccccc2)c2ccc3c4ccccc4n(-c4ccc5c(c4)CSc4nc6ccccc6n4-5)c3c2)cc1] 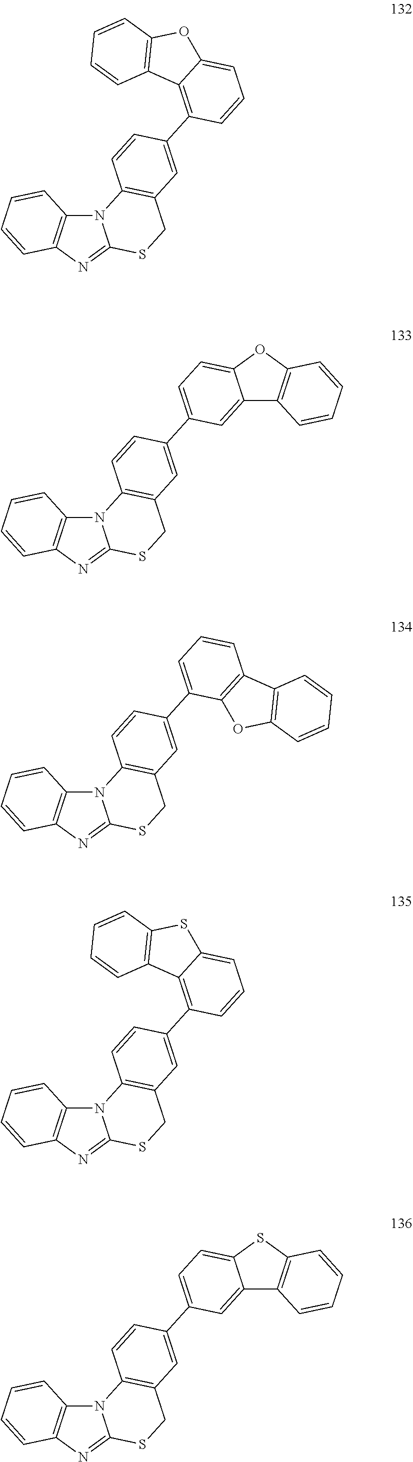 [SMILES: c1ccc2c(c1)nc1n2-c2ccc(-c3cccc4oc5ccccc5c34)cc2CS1 ; c1ccc2c(c1)nc1n2-c2ccc(-c3ccc4oc5ccccc5c4c3)cc2CS1 ; c1ccc2c(c1)nc1n2-c2ccc(-c3cccc4c3oc3ccccc34)cc2CS1 ; c1ccc2c(c1)nc1n2-c2ccc(-c3cccc4sc5ccccc5c34)cc2CS1 ; c1ccc2c(c1)nc1n2-c2ccc(-c3ccc4sc5ccccc5c4c3)cc2CS1] 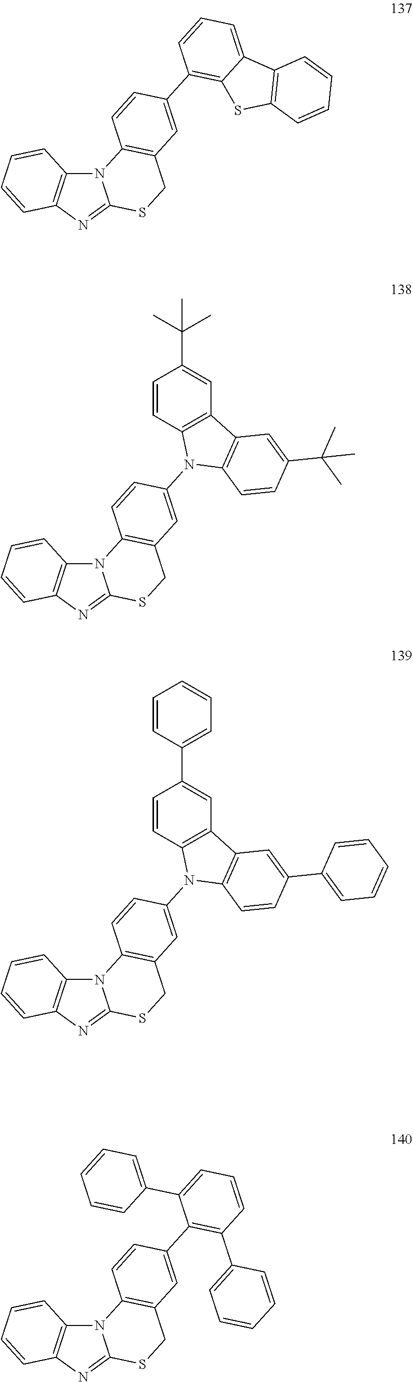 [SMILES: c1ccc2c(c1)nc1n2-c2ccc(-c3cccc4c3sc3ccccc34)cc2CS1 ; CC(C)(C)c1ccc2c(c1)c1cc(C(C)(C)C)ccc1n2-c1ccc2c(c1)CSc1nc3ccccc3n1-2 ; c1ccc(-c2ccc3c(c2)c2cc(-c4ccccc4)ccc2n3-c2ccc3c(c2)CSc2nc4ccccc4n2-3)cc1 ; c1ccc(-c2cccc(-c3ccccc3)c2-c2ccc3c(c2)CSc2nc4ccccc4n2-3)cc1] 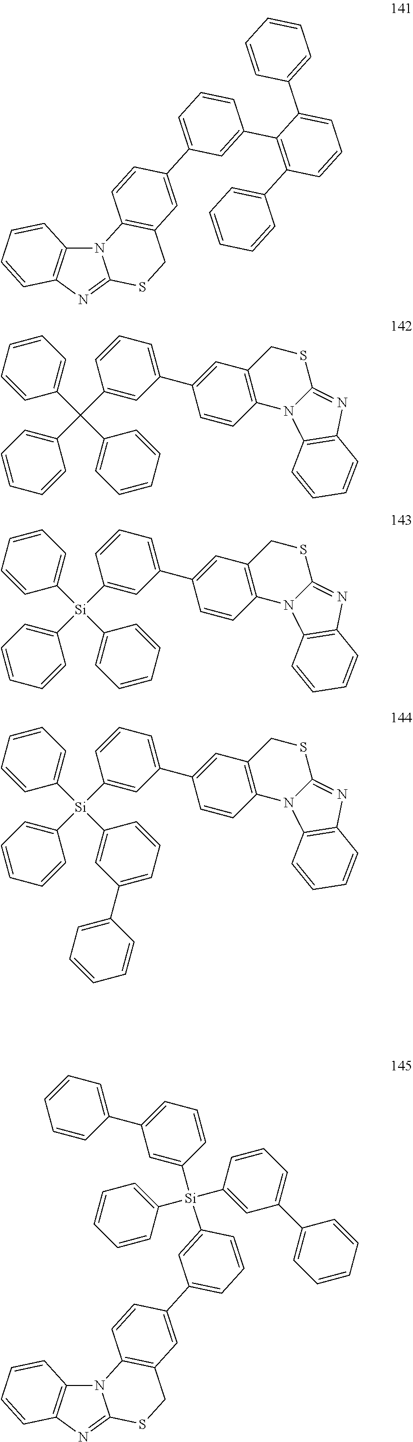 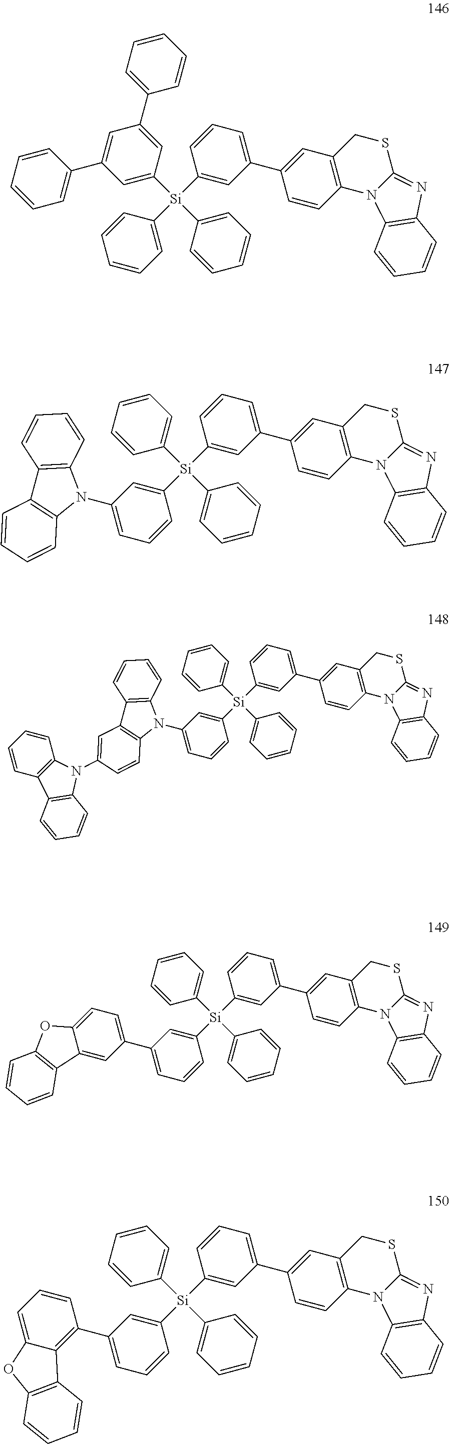 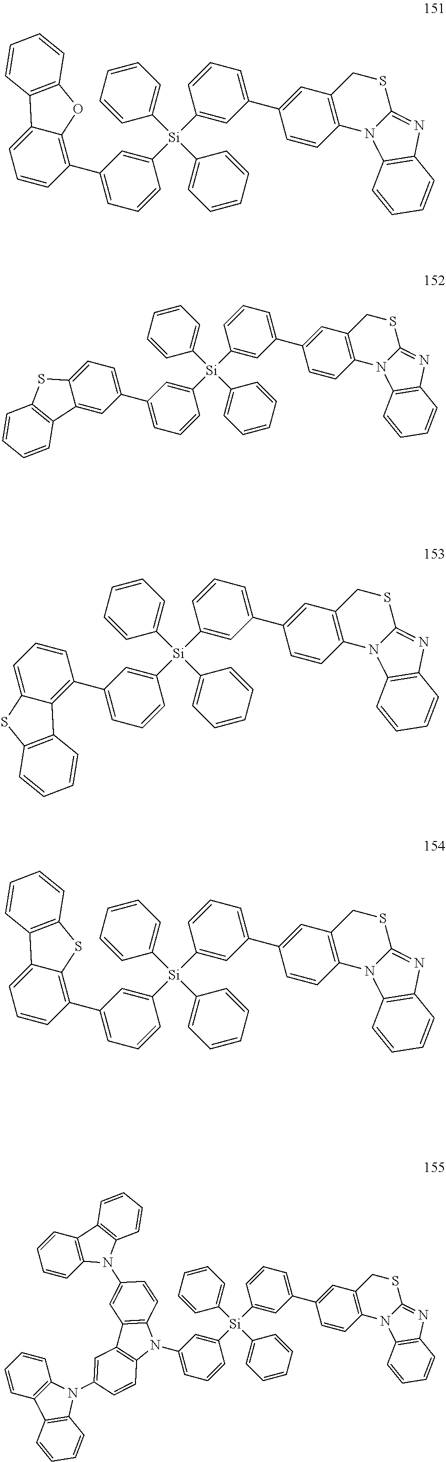 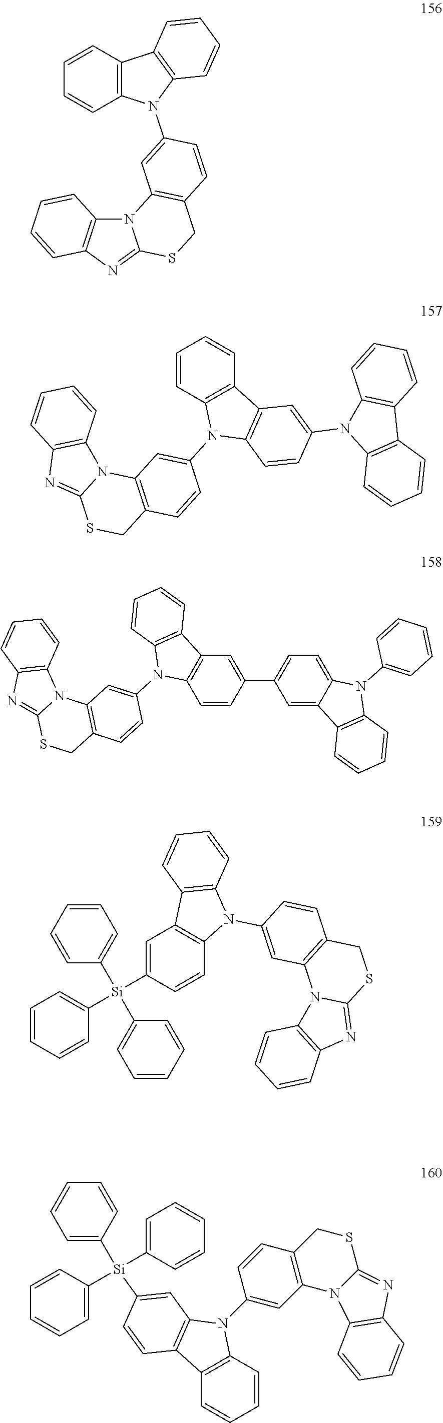 [SMILES: c1ccc2c(c1)nc1n2-c2cc(-n3c4ccccc4c4ccccc43)ccc2CS1 ; c1ccc2c(c1)nc1n2-c2cc(-n3c4ccccc4c4cc(-n5c6ccccc6c6ccccc65)ccc43)ccc2CS1 ; c1ccc(-n2c3ccccc3c3cc(-c4ccc5c(c4)c4ccccc4n5-c4ccc5c(c4)-n4c(nc6ccccc64)SC5)ccc32)cc1 ; c1ccc([Si](c2ccccc2)(c2ccccc2)c2ccc3c(c2)c2ccccc2n3-c2ccc3c(c2)-n2c(nc4ccccc42)SC3)cc1 ; c1ccc([Si](c2ccccc2)(c2ccccc2)c2ccc3c4ccccc4n(-c4ccc5c(c4)-n4c(nc6ccccc64)SC5)c3c2)cc1] 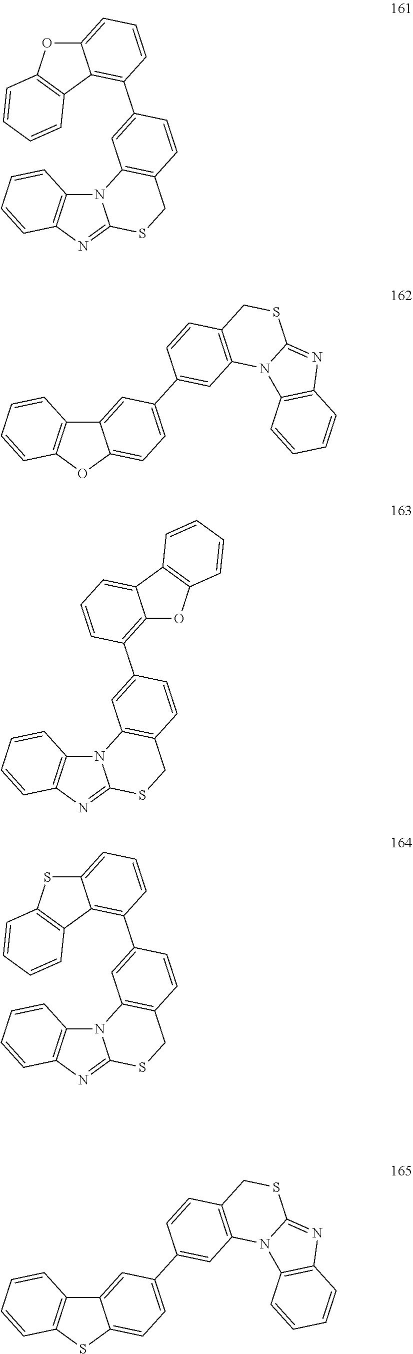 [SMILES: c1ccc2c(c1)nc1n2-c2cc(-c3cccc4oc5ccccc5c34)ccc2CS1 ; c1ccc2c(c1)nc1n2-c2cc(-c3ccc4oc5ccccc5c4c3)ccc2CS1 ; c1ccc2c(c1)nc1n2-c2cc(-c3cccc4c3oc3ccccc34)ccc2CS1 ; c1ccc2c(c1)nc1n2-c2cc(-c3cccc4sc5ccccc5c34)ccc2CS1 ; c1ccc2c(c1)nc1n2-c2cc(-c3ccc4sc5ccccc5c4c3)ccc2CS1] 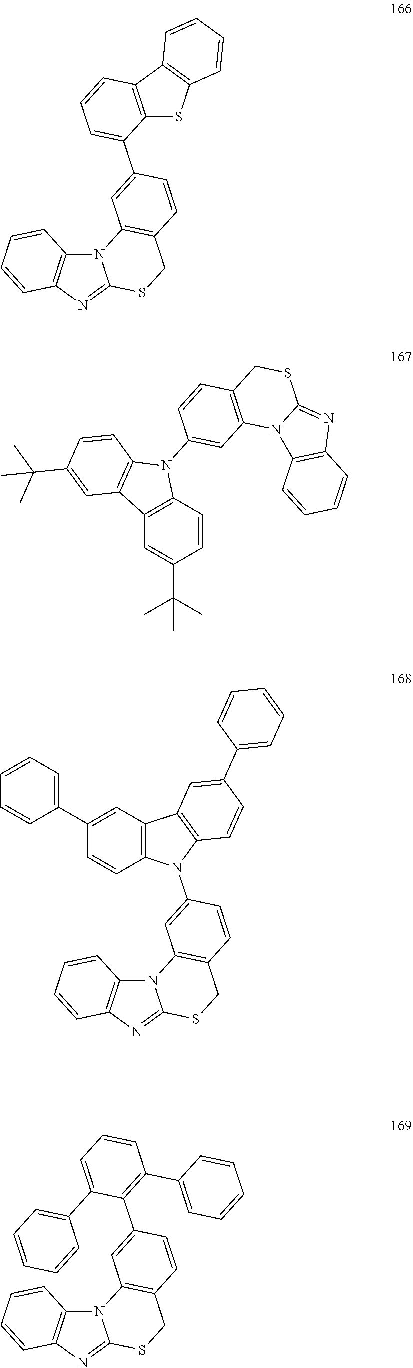 [SMILES: c1ccc2c(c1)nc1n2-c2cc(-c3cccc4c3sc3ccccc34)ccc2CS1 ; CC(C)(C)c1ccc2c(c1)c1cc(C(C)(C)C)ccc1n2-c1ccc2c(c1)-n1c(nc3ccccc31)SC2 ; c1ccc(-c2ccc3c(c2)c2cc(-c4ccccc4)ccc2n3-c2ccc3c(c2)-n2c(nc4ccccc42)SC3)cc1 ; c1ccc(-c2cccc(-c3ccccc3)c2-c2ccc3c(c2)-n2c(nc4ccccc42)SC3)cc1] 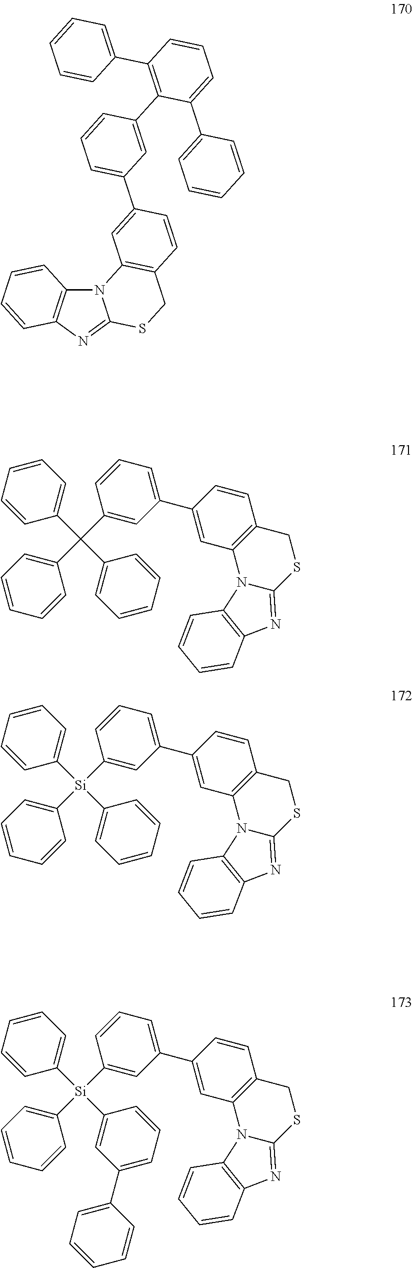 [SMILES: c1ccc(-c2cccc(-c3ccccc3)c2-c2cccc(-c3ccc4c(c3)-n3c(nc5ccccc53)SC4)c2)cc1 ; c1ccc(C(c2ccccc2)(c2ccccc2)c2cccc(-c3ccc4c(c3)-n3c(nc5ccccc53)SC4)c2)cc1 ; c1ccc([Si](c2ccccc2)(c2ccccc2)c2cccc(-c3ccc4c(c3)-n3c(nc5ccccc53)SC4)c2)cc1 ; c1ccc(-c2cccc([Si](c3ccccc3)(c3ccccc3)c3cccc(-c4ccc5c(c4)-n4c(nc6ccccc64)SC5)c3)c2)cc1] 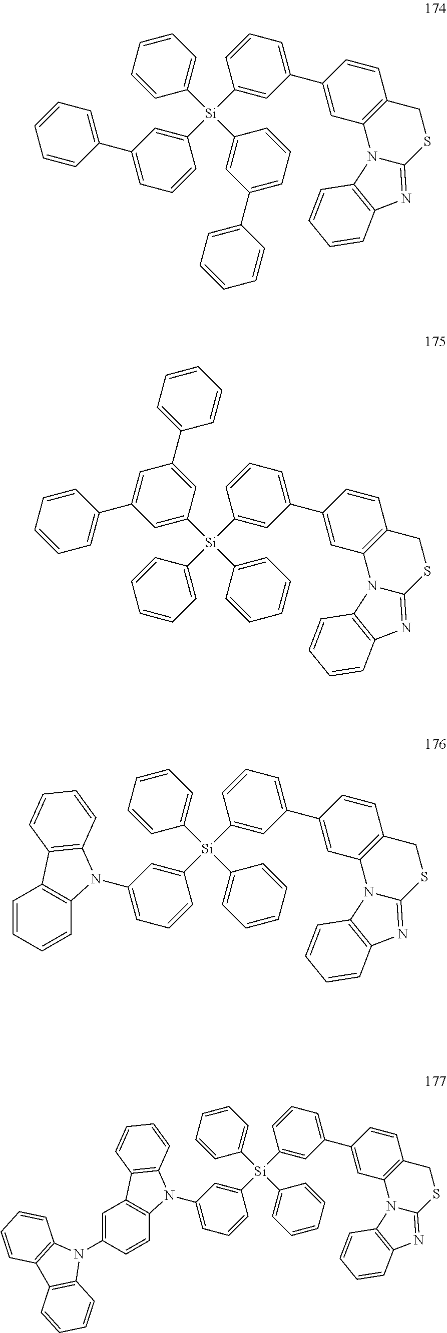 [SMILES: c1ccc(-c2cccc([Si](c3ccccc3)(c3cccc(-c4ccccc4)c3)c3cccc(-c4ccc5c(c4)-n4c(nc6ccccc64)SC5)c3)c2)cc1 ; c1ccc(-c2cc(-c3ccccc3)cc([Si](c3ccccc3)(c3ccccc3)c3cccc(-c4ccc5c(c4)-n4c(nc6ccccc64)SC5)c3)c2)cc1 ; c1ccc([Si](c2ccccc2)(c2cccc(-c3ccc4c(c3)-n3c(nc5ccccc53)SC4)c2)c2cccc(-n3c4ccccc4c4ccccc43)c2)cc1 ; c1ccc([Si](c2ccccc2)(c2cccc(-c3ccc4c(c3)-n3c(nc5ccccc53)SC4)c2)c2cccc(-n3c4ccccc4c4cc(-n5c6ccccc6c6ccccc65)ccc43)c2)cc1] 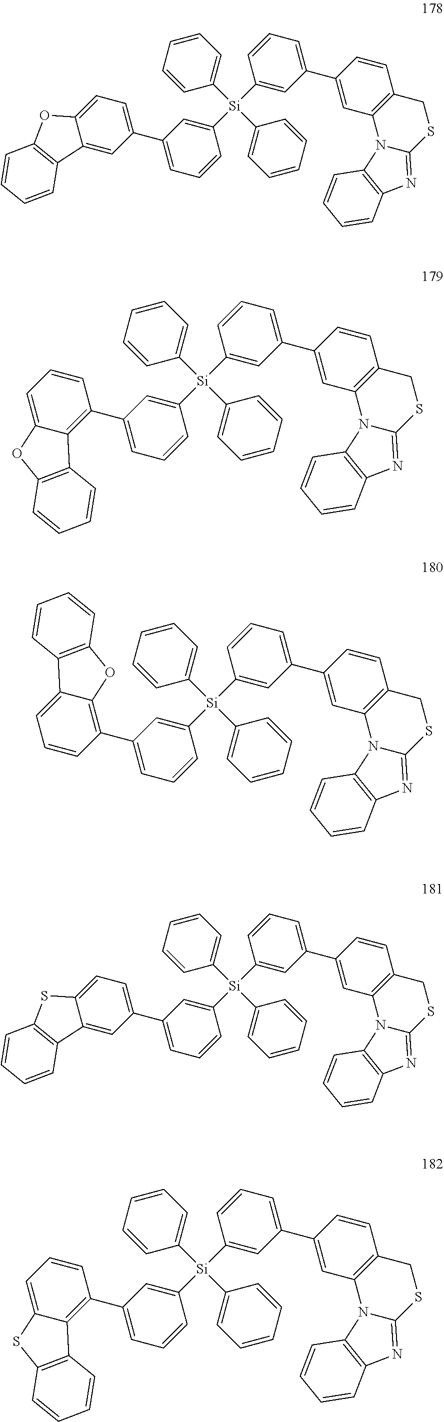 [SMILES: c1ccc([Si](c2ccccc2)(c2cccc(-c3ccc4c(c3)-n3c(nc5ccccc53)SC4)c2)c2cccc(-c3ccc4oc5ccccc5c4c3)c2)cc1 ; c1ccc([Si](c2ccccc2)(c2cccc(-c3ccc4c(c3)-n3c(nc5ccccc53)SC4)c2)c2cccc(-c3cccc4oc5ccccc5c34)c2)cc1 ; c1ccc([Si](c2ccccc2)(c2cccc(-c3ccc4c(c3)-n3c(nc5ccccc53)SC4)c2)c2cccc(-c3cccc4c3oc3ccccc34)c2)cc1 ; c1ccc([Si](c2ccccc2)(c2cccc(-c3ccc4c(c3)-n3c(nc5ccccc53)SC4)c2)c2cccc(-c3ccc4sc5ccccc5c4c3)c2)cc1 ; c1ccc([Si](c2ccccc2)(c2cccc(-c3ccc4c(c3)-n3c(nc5ccccc53)SC4)c2)c2cccc(-c3cccc4sc5ccccc5c34)c2)cc1] 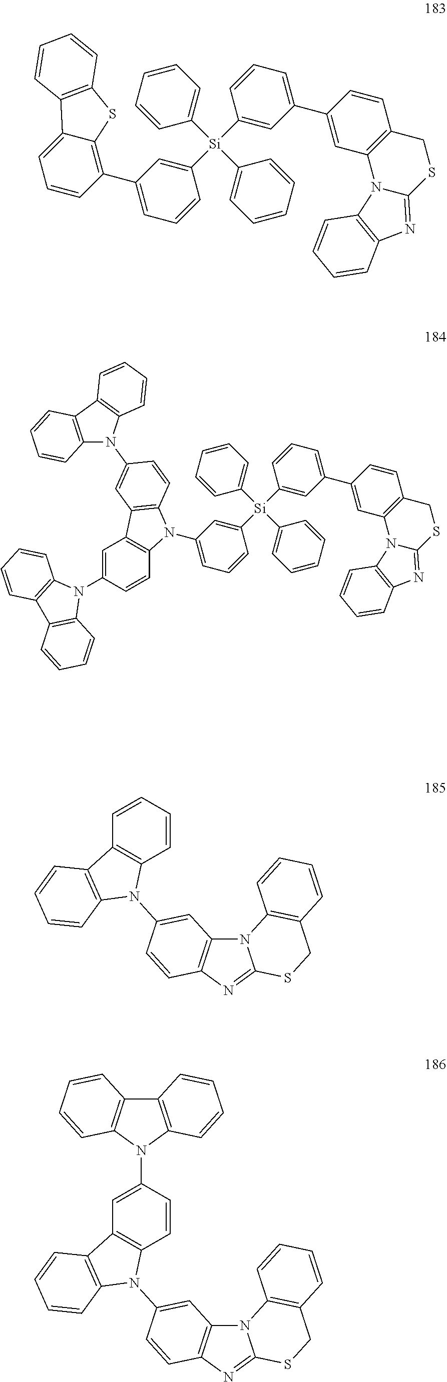 [SMILES: c1ccc([Si](c2ccccc2)(c2cccc(-c3ccc4c(c3)-n3c(nc5ccccc53)SC4)c2)c2cccc(-c3cccc4c3sc3ccccc34)c2)cc1 ; c1ccc([Si](c2ccccc2)(c2cccc(-c3ccc4c(c3)-n3c(nc5ccccc53)SC4)c2)c2cccc(-n3c4ccc(-n5c6ccccc6c6ccccc65)cc4c4cc(-n5c6ccccc6c6ccccc65)ccc43)c2)cc1 ; c1ccc2c(c1)CSc1nc3ccc(-n4c5ccccc5c5ccccc54)cc3n1-2 ; c1ccc2c(c1)CSc1nc3ccc(-n4c5ccccc5c5cc(-n6c7ccccc7c7ccccc76)ccc54)cc3n1-2] 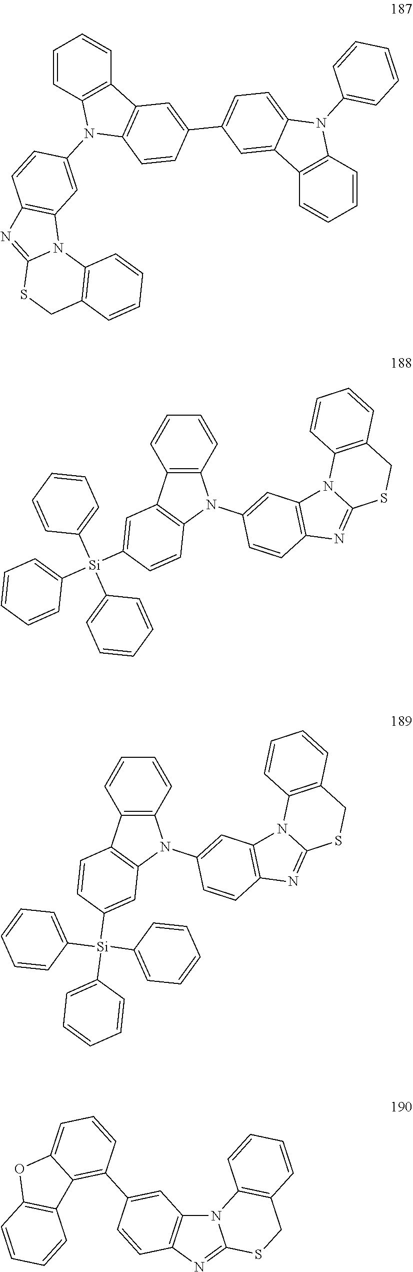 [SMILES: c1ccc(-n2c3ccccc3c3cc(-c4ccc5c(c4)c4ccccc4n5-c4ccc5nc6n(c5c4)-c4ccccc4CS6)ccc32)cc1 ; c1ccc([Si](c2ccccc2)(c2ccccc2)c2ccc3c(c2)c2ccccc2n3-c2ccc3nc4n(c3c2)-c2ccccc2CS4)cc1 ; c1ccc([Si](c2ccccc2)(c2ccccc2)c2ccc3c4ccccc4n(-c4ccc5nc6n(c5c4)-c4ccccc4CS6)c3c2)cc1 ; c1ccc2c(c1)CSc1nc3ccc(-c4cccc5oc6ccccc6c45)cc3n1-2] 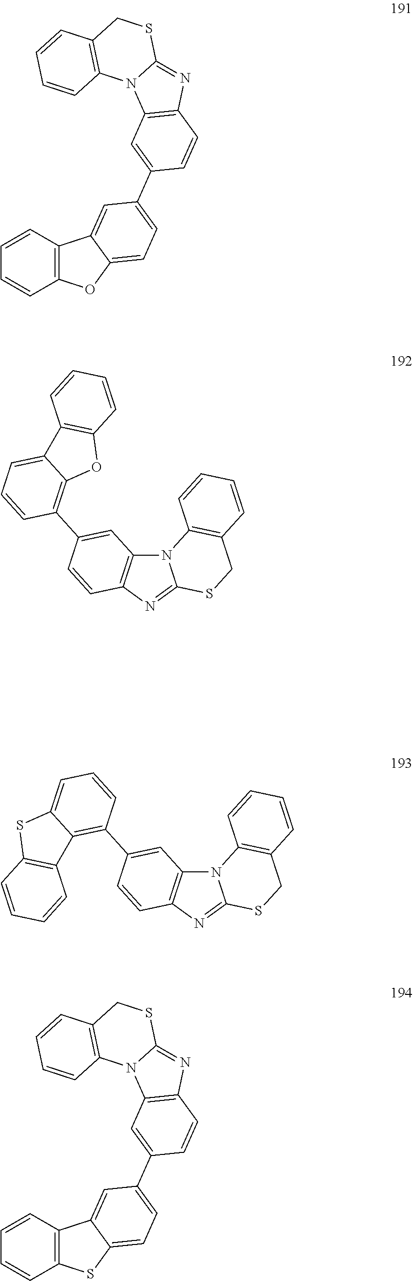 [SMILES: c1ccc2c(c1)CSc1nc3ccc(-c4ccc5oc6ccccc6c5c4)cc3n1-2 ; c1ccc2c(c1)CSc1nc3ccc(-c4cccc5c4oc4ccccc45)cc3n1-2 ; c1ccc2c(c1)CSc1nc3ccc(-c4cccc5sc6ccccc6c45)cc3n1-2 ; c1ccc2c(c1)CSc1nc3ccc(-c4ccc5sc6ccccc6c5c4)cc3n1-2] 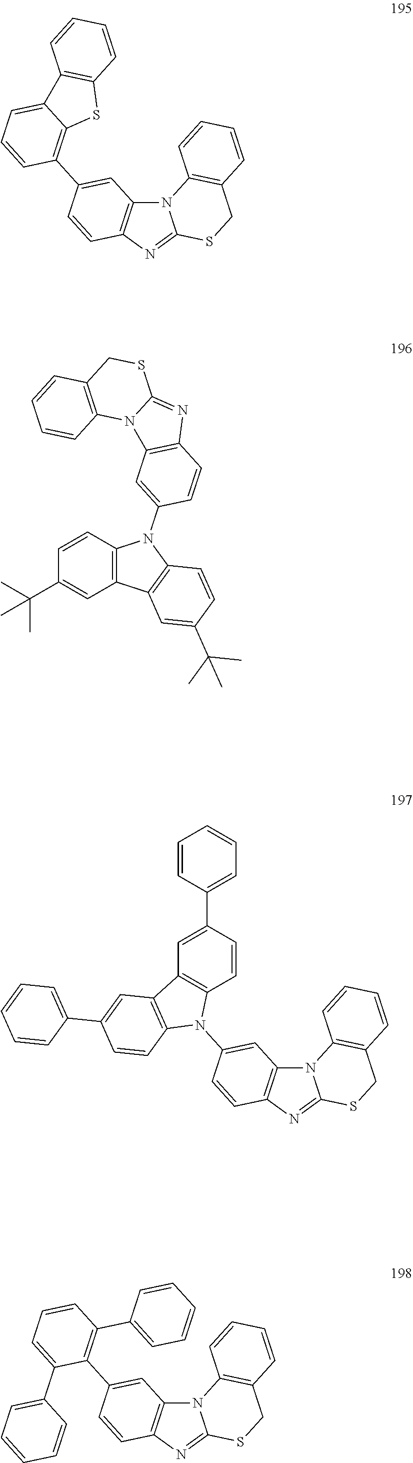 [SMILES: c1ccc2c(c1)CSc1nc3ccc(-c4cccc5c4sc4ccccc45)cc3n1-2 ; CC(C)(C)c1ccc2c(c1)c1cc(C(C)(C)C)ccc1n2-c1ccc2nc3n(c2c1)-c1ccccc1CS3 ; c1ccc(-c2ccc3c(c2)c2cc(-c4ccccc4)ccc2n3-c2ccc3nc4n(c3c2)-c2ccccc2CS4)cc1 ; c1ccc(-c2cccc(-c3ccccc3)c2-c2ccc3nc4n(c3c2)-c2ccccc2CS4)cc1] 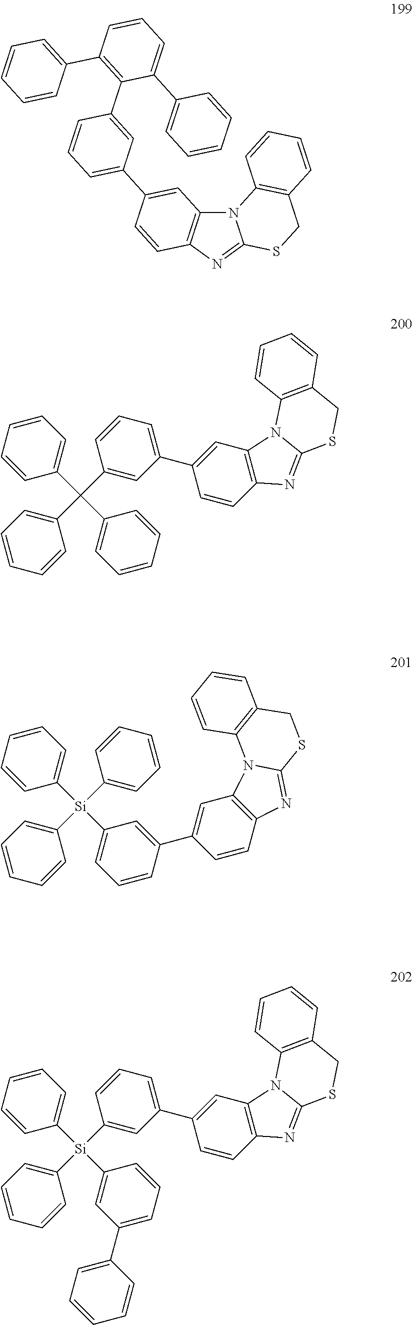 [SMILES: c1ccc(-c2cccc(-c3ccccc3)c2-c2cccc(-c3ccc4nc5n(c4c3)-c3ccccc3CS5)c2)cc1 ; c1ccc(C(c2ccccc2)(c2ccccc2)c2cccc(-c3ccc4nc5n(c4c3)-c3ccccc3CS5)c2)cc1 ; c1ccc([Si](c2ccccc2)(c2ccccc2)c2cccc(-c3ccc4nc5n(c4c3)-c3ccccc3CS5)c2)cc1 ; c1ccc(-c2cccc([Si](c3ccccc3)(c3ccccc3)c3cccc(-c4ccc5nc6n(c5c4)-c4ccccc4CS6)c3)c2)cc1] 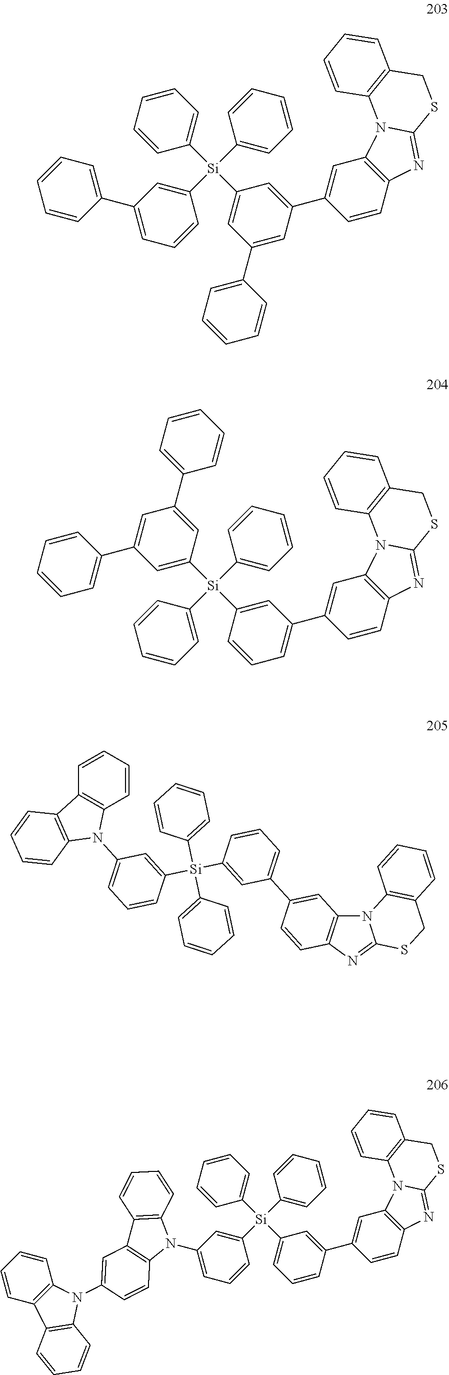 [SMILES: c1ccc(-c2cccc([Si](c3ccccc3)(c3ccccc3)c3cc(-c4ccccc4)cc(-c4ccc5nc6n(c5c4)-c4ccccc4CS6)c3)c2)cc1 ; c1ccc(-c2cc(-c3ccccc3)cc([Si](c3ccccc3)(c3ccccc3)c3cccc(-c4ccc5nc6n(c5c4)-c4ccccc4CS6)c3)c2)cc1 ; c1ccc([Si](c2ccccc2)(c2cccc(-c3ccc4nc5n(c4c3)-c3ccccc3CS5)c2)c2cccc(-n3c4ccccc4c4ccccc43)c2)cc1 ; c1ccc([Si](c2ccccc2)(c2cccc(-c3ccc4nc5n(c4c3)-c3ccccc3CS5)c2)c2cccc(-n3c4ccccc4c4cc(-n5c6ccccc6c6ccccc65)ccc43)c2)cc1] 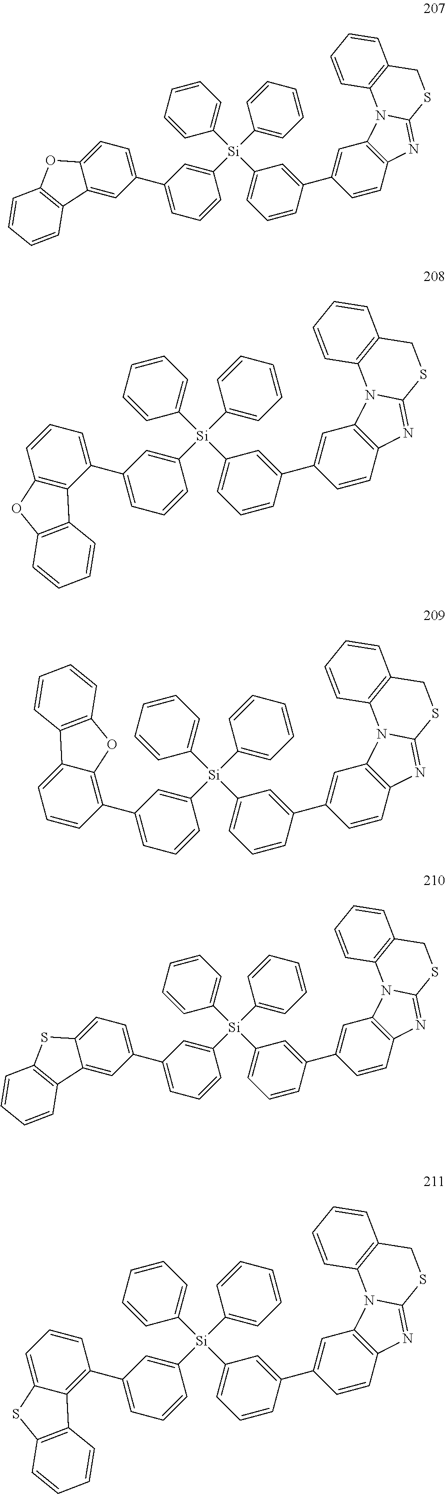 [SMILES: c1ccc([Si](c2ccccc2)(c2cccc(-c3ccc4oc5ccccc5c4c3)c2)c2cccc(-c3ccc4nc5n(c4c3)-c3ccccc3CS5)c2)cc1 ; c1ccc([Si](c2ccccc2)(c2cccc(-c3ccc4nc5n(c4c3)-c3ccccc3CS5)c2)c2cccc(-c3cccc4oc5ccccc5c34)c2)cc1 ; c1ccc([Si](c2ccccc2)(c2cccc(-c3ccc4nc5n(c4c3)-c3ccccc3CS5)c2)c2cccc(-c3cccc4c3oc3ccccc34)c2)cc1 ; c1ccc([Si](c2ccccc2)(c2cccc(-c3ccc4sc5ccccc5c4c3)c2)c2cccc(-c3ccc4nc5n(c4c3)-c3ccccc3CS5)c2)cc1 ; c1ccc([Si](c2ccccc2)(c2cccc(-c3ccc4nc5n(c4c3)-c3ccccc3CS5)c2)c2cccc(-c3cccc4sc5ccccc5c34)c2)cc1] 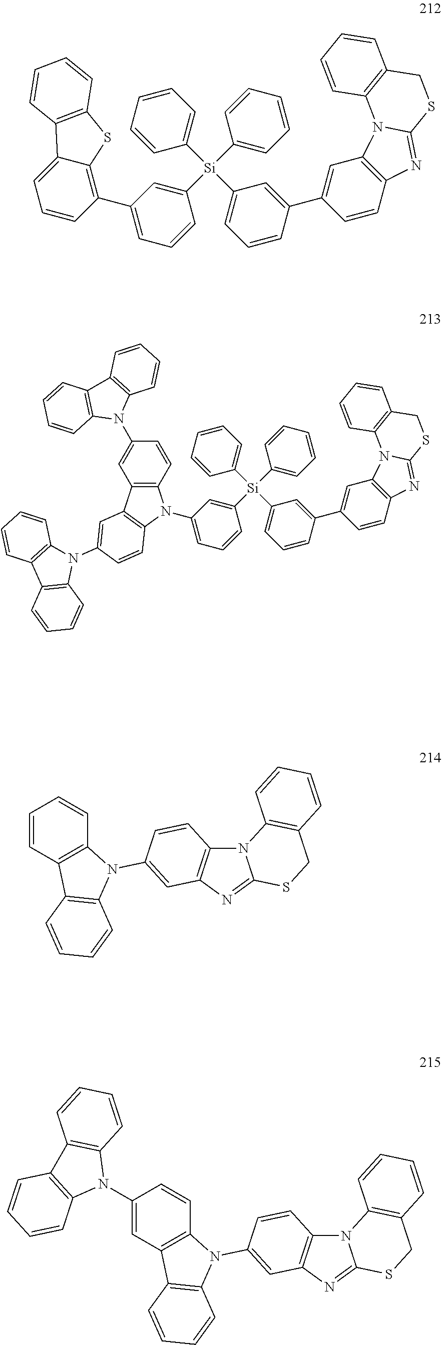 [SMILES: c1ccc([Si](c2ccccc2)(c2cccc(-c3ccc4nc5n(c4c3)-c3ccccc3CS5)c2)c2cccc(-c3cccc4c3sc3ccccc34)c2)cc1 ; c1ccc([Si](c2ccccc2)(c2cccc(-c3ccc4nc5n(c4c3)-c3ccccc3CS5)c2)c2cccc(-n3c4ccc(-n5c6ccccc6c6ccccc65)cc4c4cc(-n5c6ccccc6c6ccccc65)ccc43)c2)cc1 ; c1ccc2c(c1)CSc1nc3cc(-n4c5ccccc5c5ccccc54)ccc3n1-2 ; c1ccc2c(c1)CSc1nc3cc(-n4c5ccccc5c5cc(-n6c7ccccc7c7ccccc76)ccc54)ccc3n1-2] 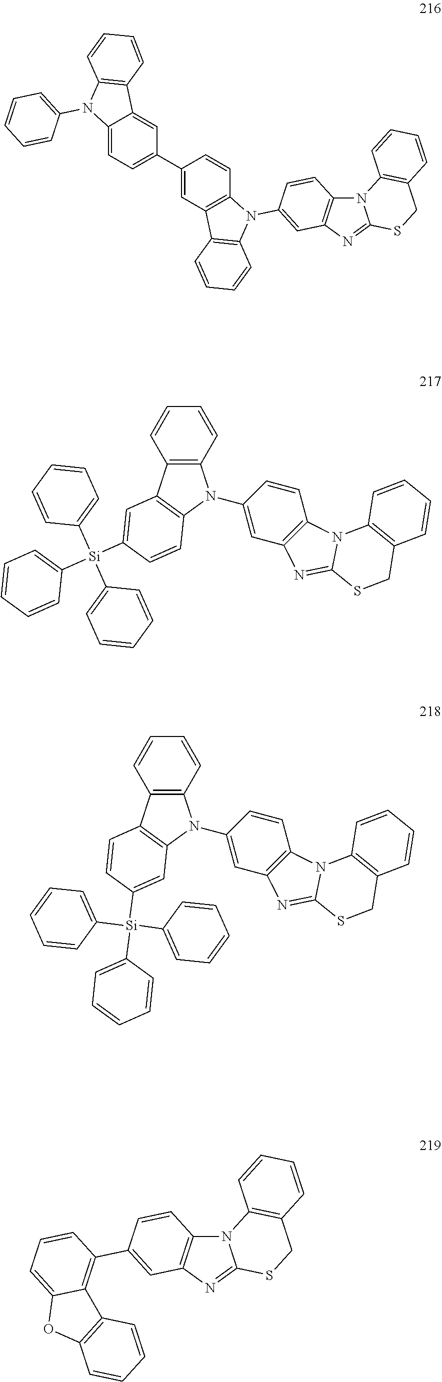 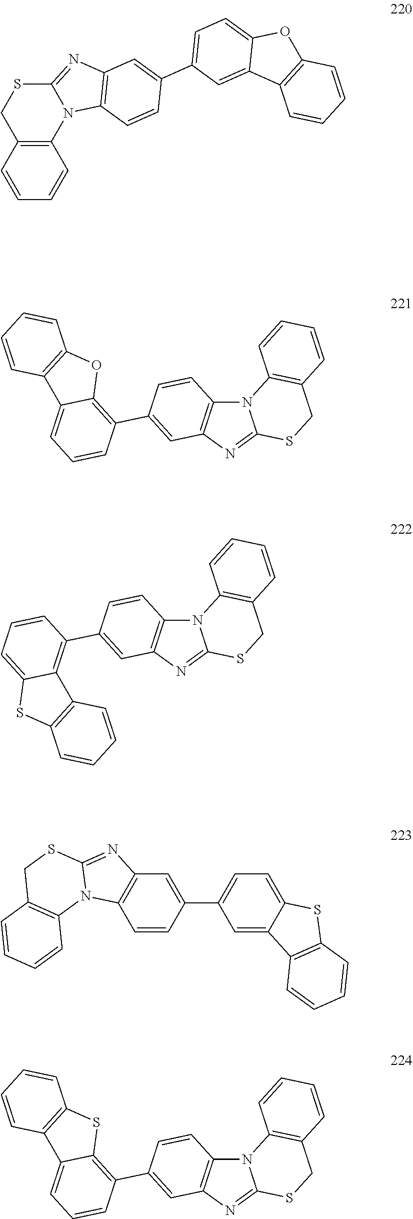 [SMILES: c1ccc2c(c1)CSc1nc3cc(-c4ccc5oc6ccccc6c5c4)ccc3n1-2 ; c1ccc2c(c1)CSc1nc3cc(-c4cccc5c4oc4ccccc45)ccc3n1-2 ; c1ccc2c(c1)CSc1nc3cc(-c4cccc5sc6ccccc6c45)ccc3n1-2 ; c1ccc2c(c1)CSc1nc3cc(-c4ccc5sc6ccccc6c5c4)ccc3n1-2 ; c1ccc2c(c1)CSc1nc3cc(-c4cccc5c4sc4ccccc45)ccc3n1-2] 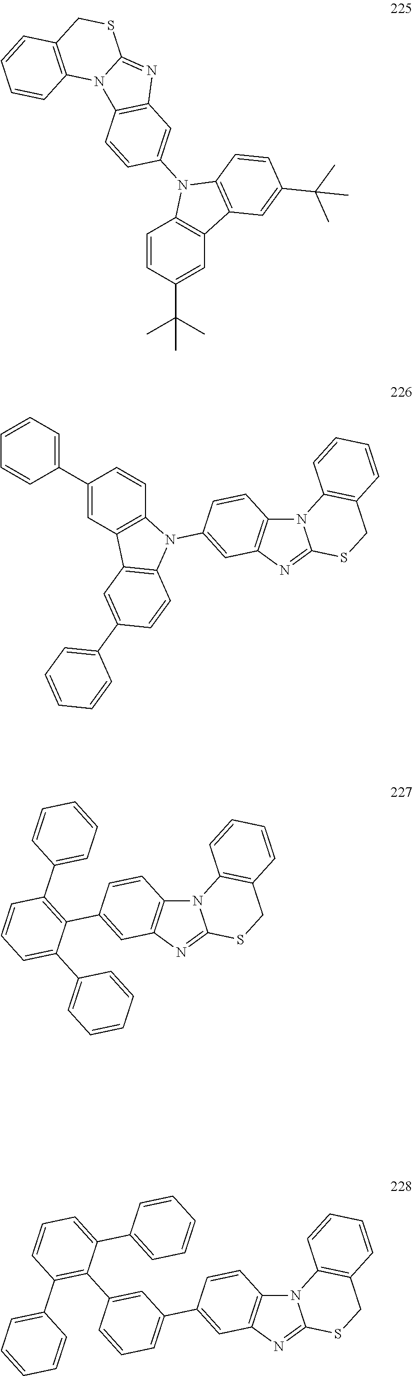 [SMILES: CC(C)(C)c1ccc2c(c1)c1cc(C(C)(C)C)ccc1n2-c1ccc2c(c1)nc1n2-c2ccccc2CS1 ; c1ccc(-c2ccc3c(c2)c2cc(-c4ccccc4)ccc2n3-c2ccc3c(c2)nc2n3-c3ccccc3CS2)cc1 ; c1ccc(-c2cccc(-c3ccccc3)c2-c2ccc3c(c2)nc2n3-c3ccccc3CS2)cc1 ; c1ccc(-c2cccc(-c3ccccc3)c2-c2cccc(-c3ccc4c(c3)nc3n4-c4ccccc4CS3)c2)cc1] 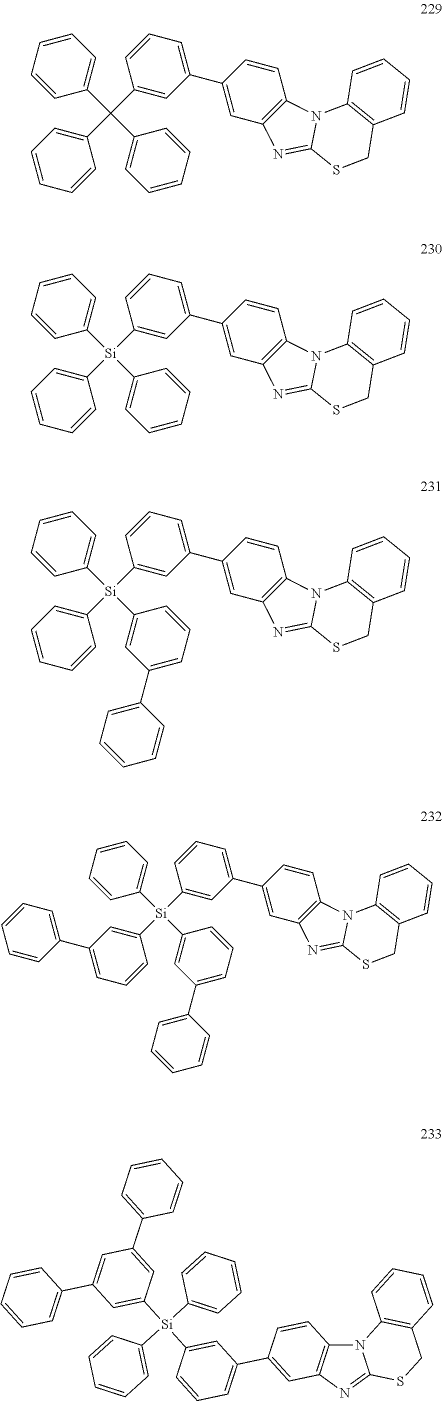 [SMILES: c1ccc(C(c2ccccc2)(c2ccccc2)c2cccc(-c3ccc4c(c3)nc3n4-c4ccccc4CS3)c2)cc1 ; c1ccc([Si](c2ccccc2)(c2ccccc2)c2cccc(-c3ccc4c(c3)nc3n4-c4ccccc4CS3)c2)cc1 ; c1ccc(-c2cccc([Si](c3ccccc3)(c3ccccc3)c3cccc(-c4ccc5c(c4)nc4n5-c5ccccc5CS4)c3)c2)cc1 ; c1ccc(-c2cccc([Si](c3ccccc3)(c3cccc(-c4ccccc4)c3)c3cccc(-c4ccc5c(c4)nc4n5-c5ccccc5CS4)c3)c2)cc1 ; c1ccc(-c2cc(-c3ccccc3)cc([Si](c3ccccc3)(c3ccccc3)c3cccc(-c4ccc5c(c4)nc4n5-c5ccccc5CS4)c3)c2)cc1] 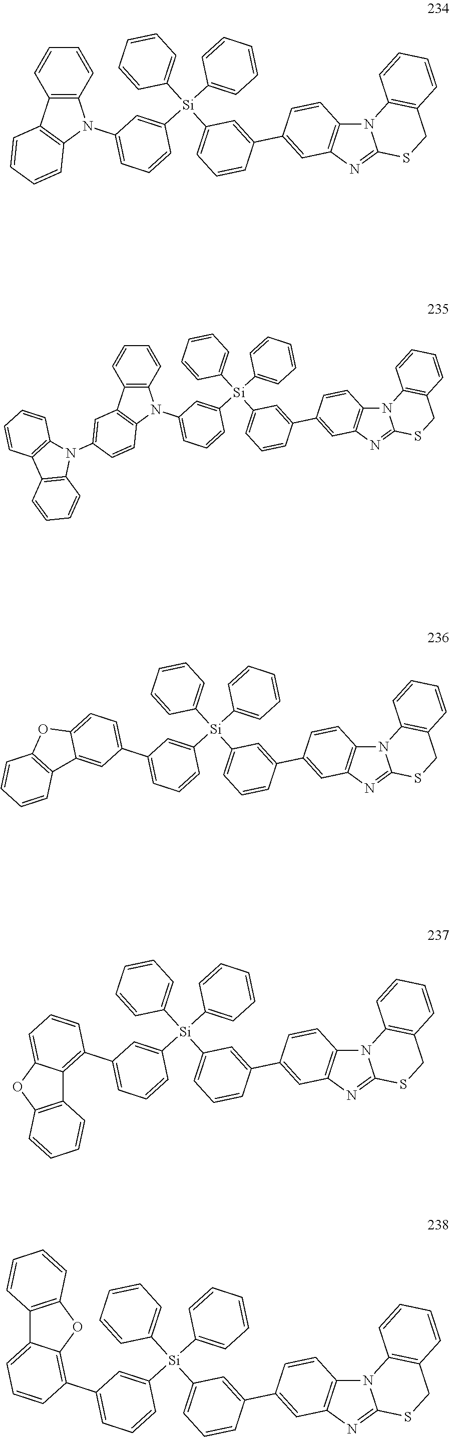 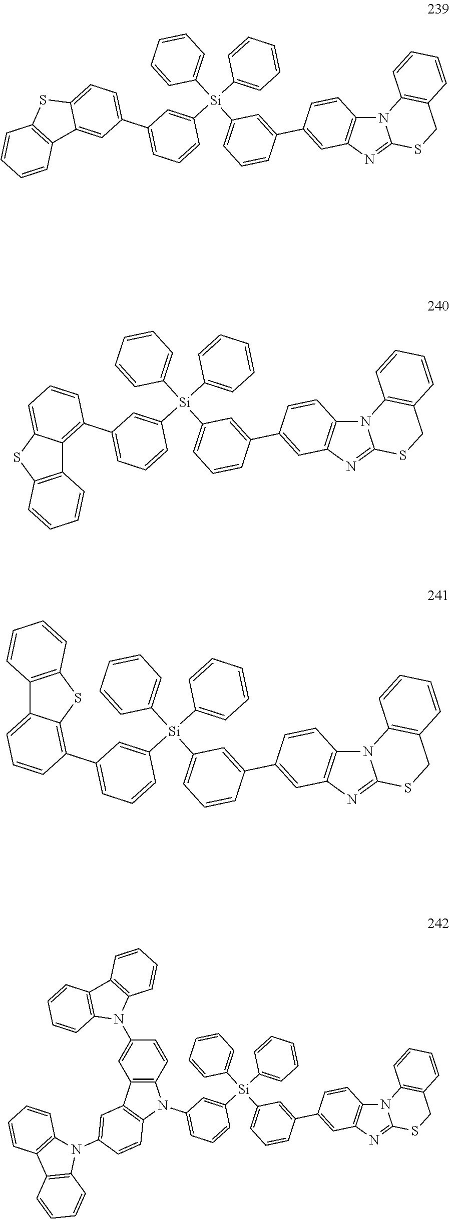 [SMILES: c1ccc([Si](c2ccccc2)(c2cccc(-c3ccc4c(c3)nc3n4-c4ccccc4CS3)c2)c2cccc(-c3ccc4sc5ccccc5c4c3)c2)cc1 ; c1ccc([Si](c2ccccc2)(c2cccc(-c3ccc4c(c3)nc3n4-c4ccccc4CS3)c2)c2cccc(-c3cccc4sc5ccccc5c34)c2)cc1 ; c1ccc([Si](c2ccccc2)(c2cccc(-c3ccc4c(c3)nc3n4-c4ccccc4CS3)c2)c2cccc(-c3cccc4c3sc3ccccc34)c2)cc1 ; c1ccc([Si](c2ccccc2)(c2cccc(-c3ccc4c(c3)nc3n4-c4ccccc4CS3)c2)c2cccc(-n3c4ccc(-n5c6ccccc6c6ccccc65)cc4c4cc(-n5c6ccccc6c6ccccc65)ccc43)c2)cc1] 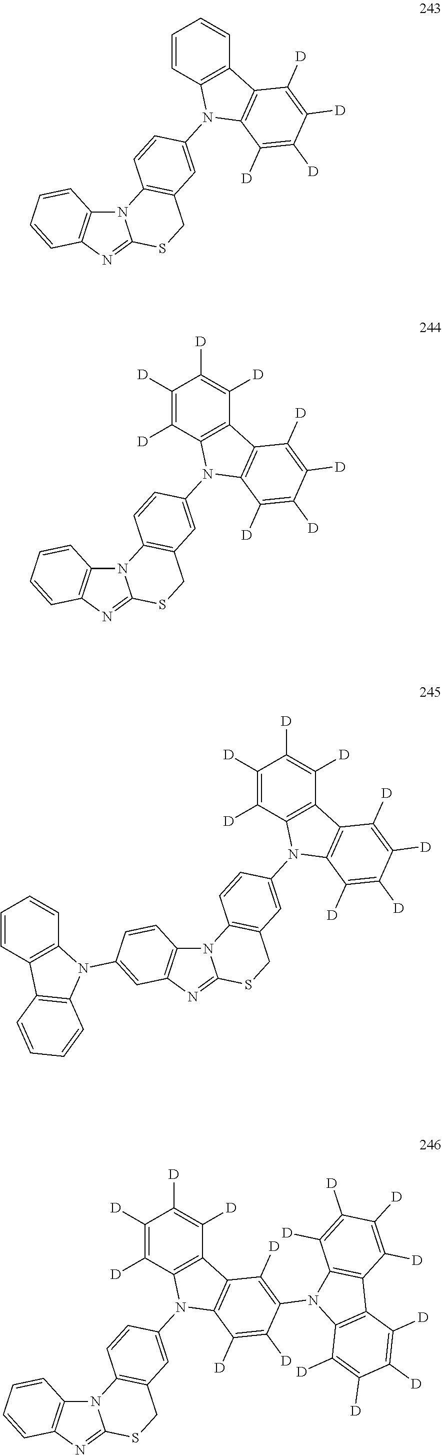 [SMILES: [2H]c1c([2H])c([2H])c2c(c1[2H])c1ccccc1n2-c1ccc2c(c1)CSc1nc3ccccc3n1-2 ; [2H]c1c([2H])c([2H])c2c(c1[2H])c1c([2H])c([2H])c([2H])c([2H])c1n2-c1ccc2c(c1)CSc1nc3ccccc3n1-2 ; [2H]c1c([2H])c([2H])c2c(c1[2H])c1c([2H])c([2H])c([2H])c([2H])c1n2-c1ccc2c(c1)CSc1nc3cc(-n4c5ccccc5c5ccccc54)ccc3n1-2 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc2c(c1)CSc1nc3ccccc3n1-2] 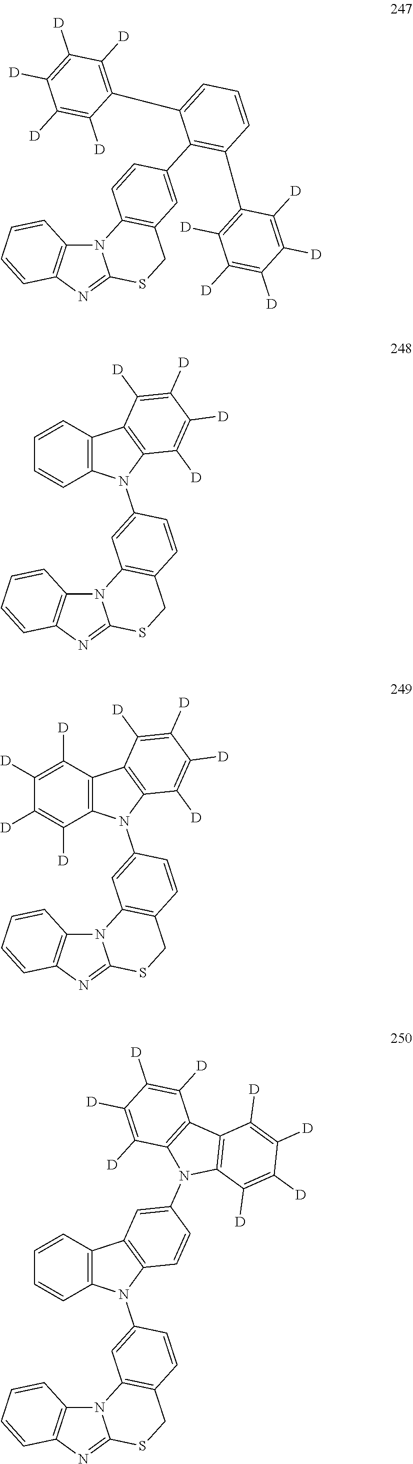 [SMILES: [2H]c1c([2H])c([2H])c(-c2cccc(-c3c([2H])c([2H])c([2H])c([2H])c3[2H])c2-c2ccc3c(c2)CSc2nc4ccccc4n2-3)c([2H])c1[2H] ; [2H]c1c([2H])c([2H])c2c(c1[2H])c1ccccc1n2-c1ccc2c(c1)-n1c(nc3ccccc31)SC2 ; [2H]c1c([2H])c([2H])c2c(c1[2H])c1c([2H])c([2H])c([2H])c([2H])c1n2-c1ccc2c(c1)-n1c(nc3ccccc31)SC2 ; [2H]c1c([2H])c([2H])c2c(c1[2H])c1c([2H])c([2H])c([2H])c([2H])c1n2-c1ccc2c(c1)c1ccccc1n2-c1ccc2c(c1)-n1c(nc3ccccc31)SC2] 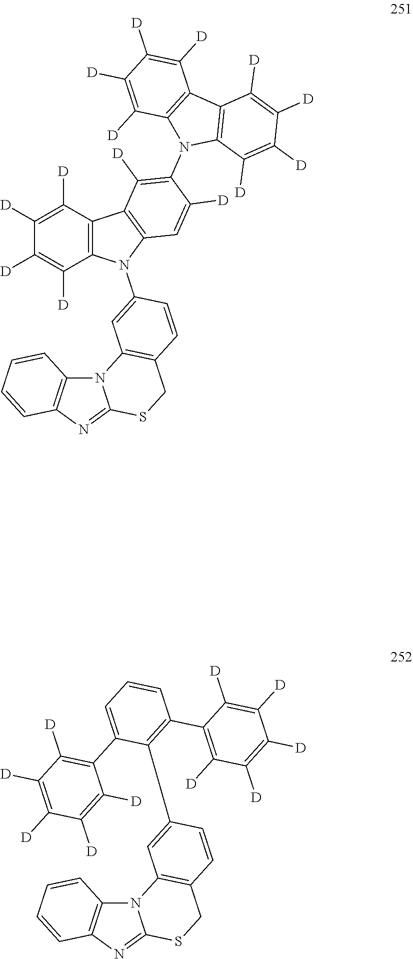 [SMILES: [2H]c1cc2c(c([2H])c1-n1c3c([2H])c([2H])c([2H])c([2H])c3c3c([2H])c([2H])c([2H])c([2H])c31)c1c([2H])c([2H])c([2H])c([2H])c1n2-c1ccc2c(c1)-n1c(nc3ccccc31)SC2 ; [2H]c1c([2H])c([2H])c(-c2cccc(-c3c([2H])c([2H])c([2H])c([2H])c3[2H])c2-c2ccc3c(c2)-n2c(nc4ccccc42)SC3)c([2H])c1[2H]]
Group II 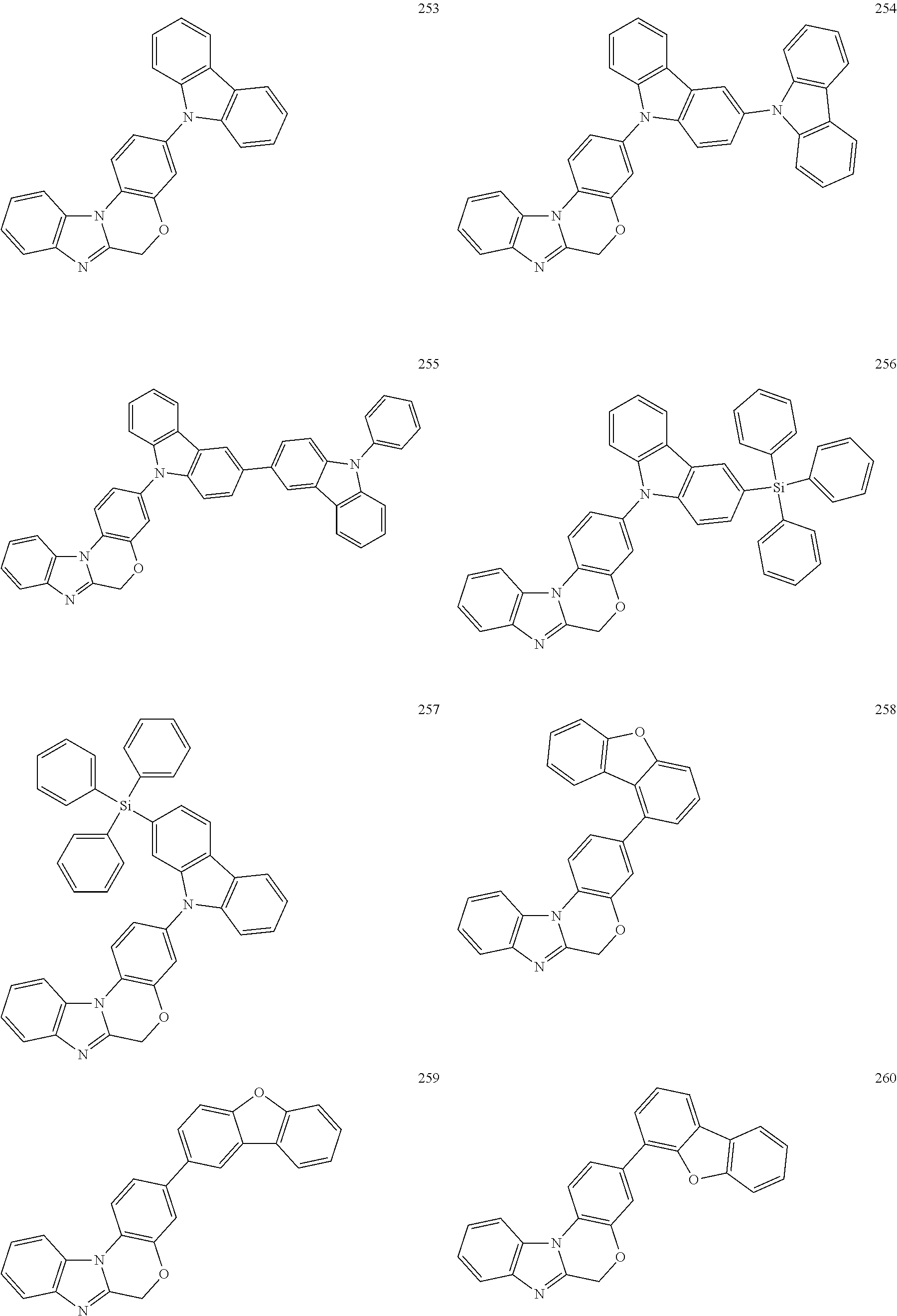 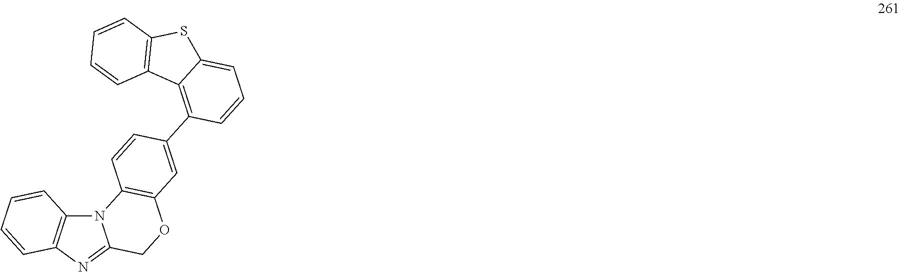 [SMILES: c1ccc2c(c1)nc1n2-c2ccc(-c3cccc4sc5ccccc5c34)cc2OC1] 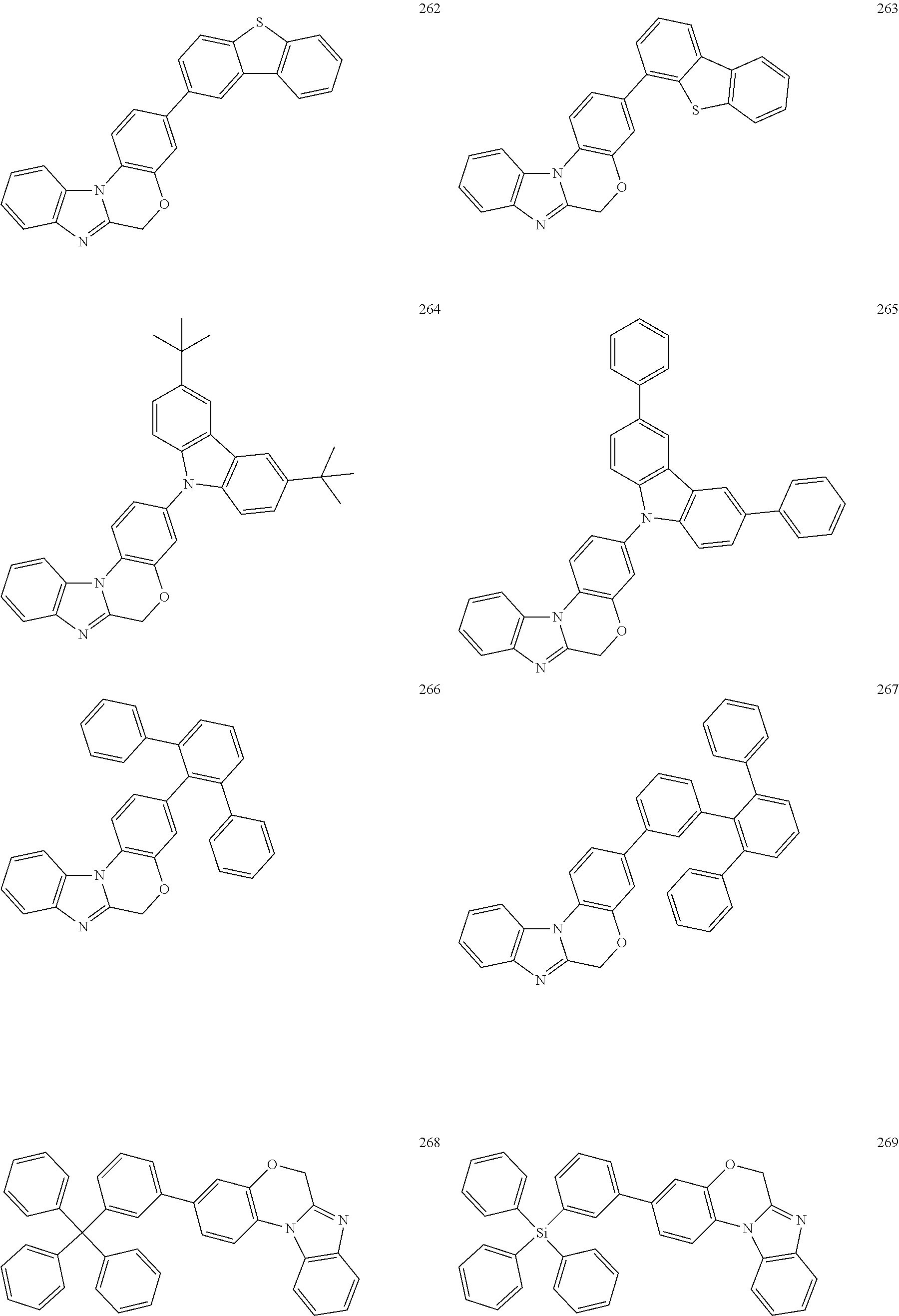 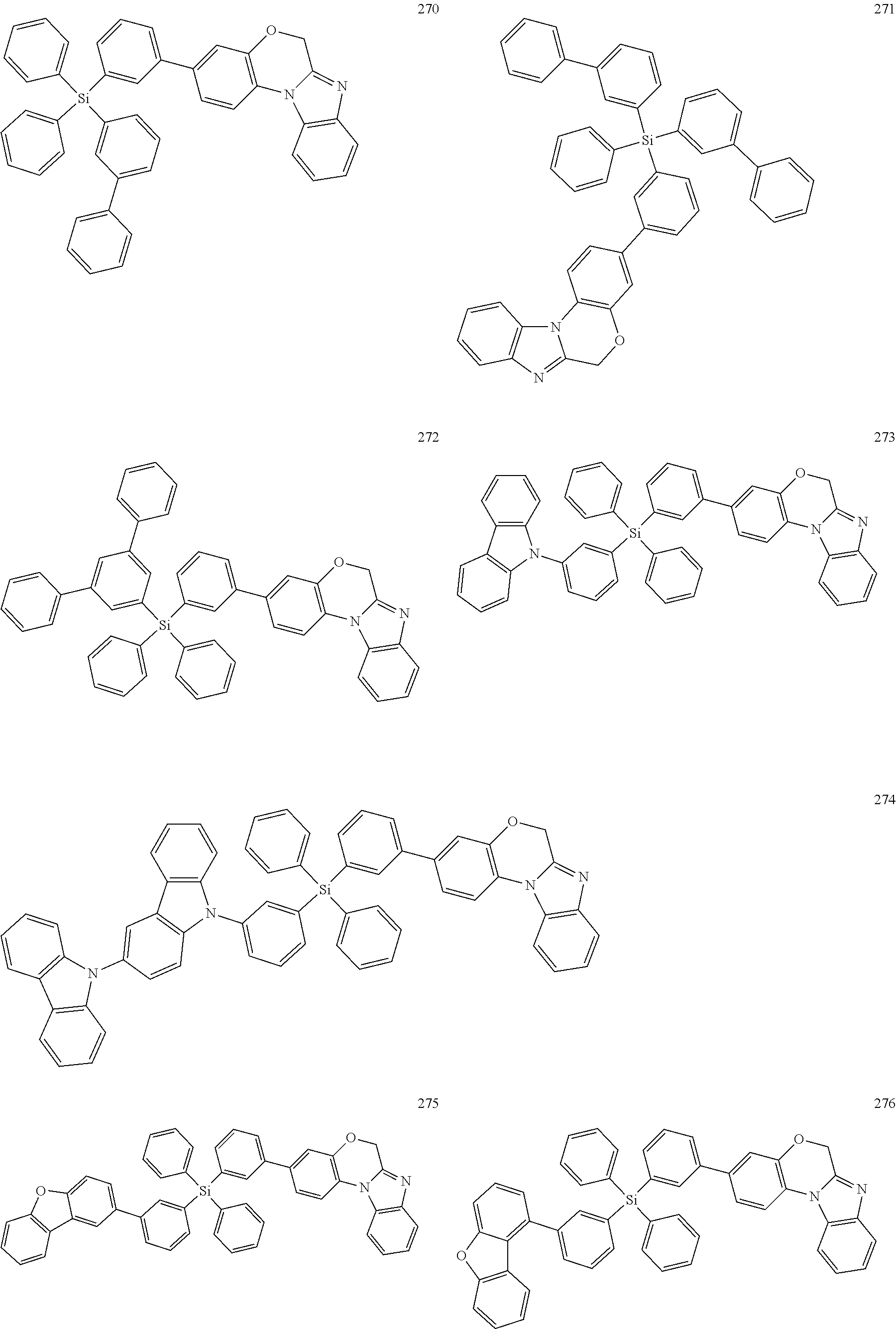 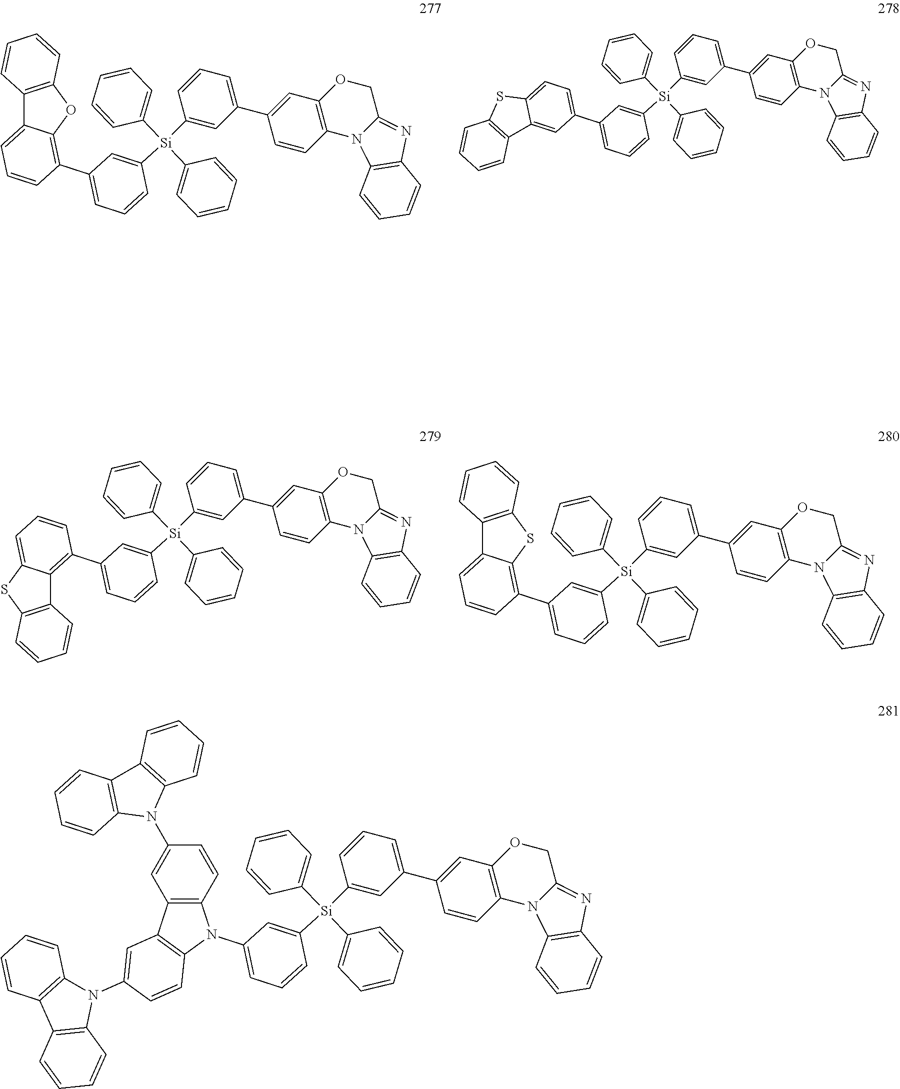 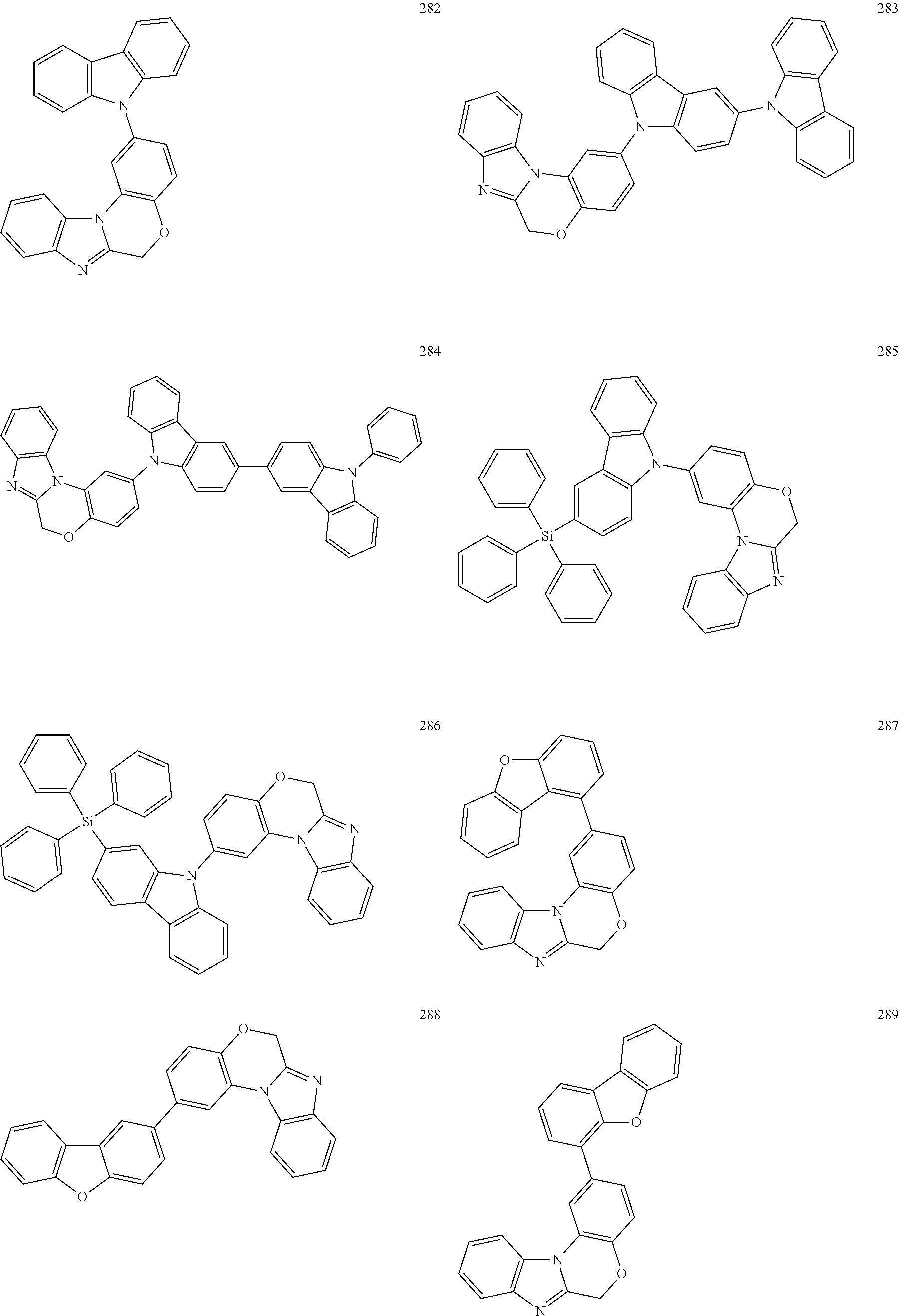 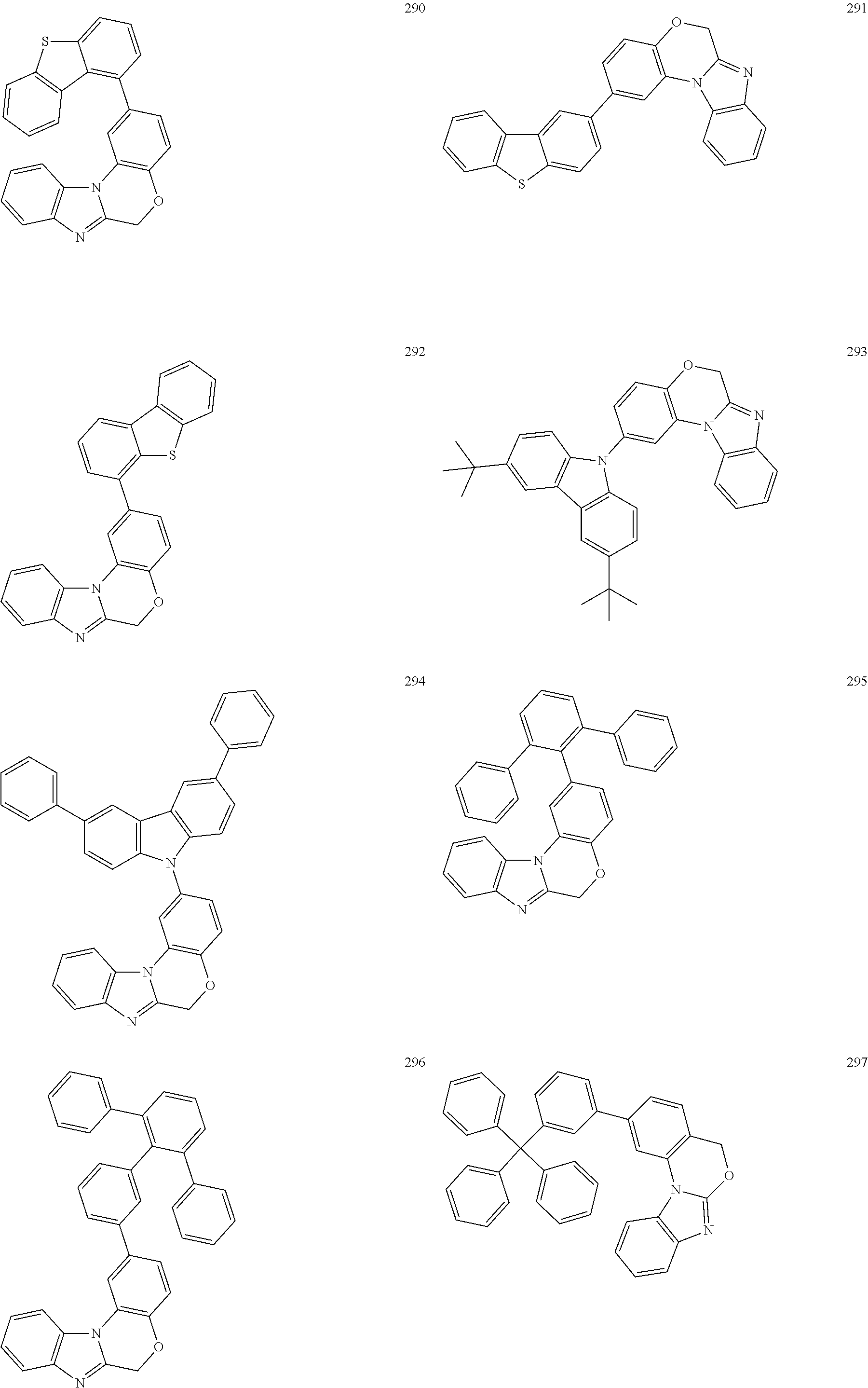 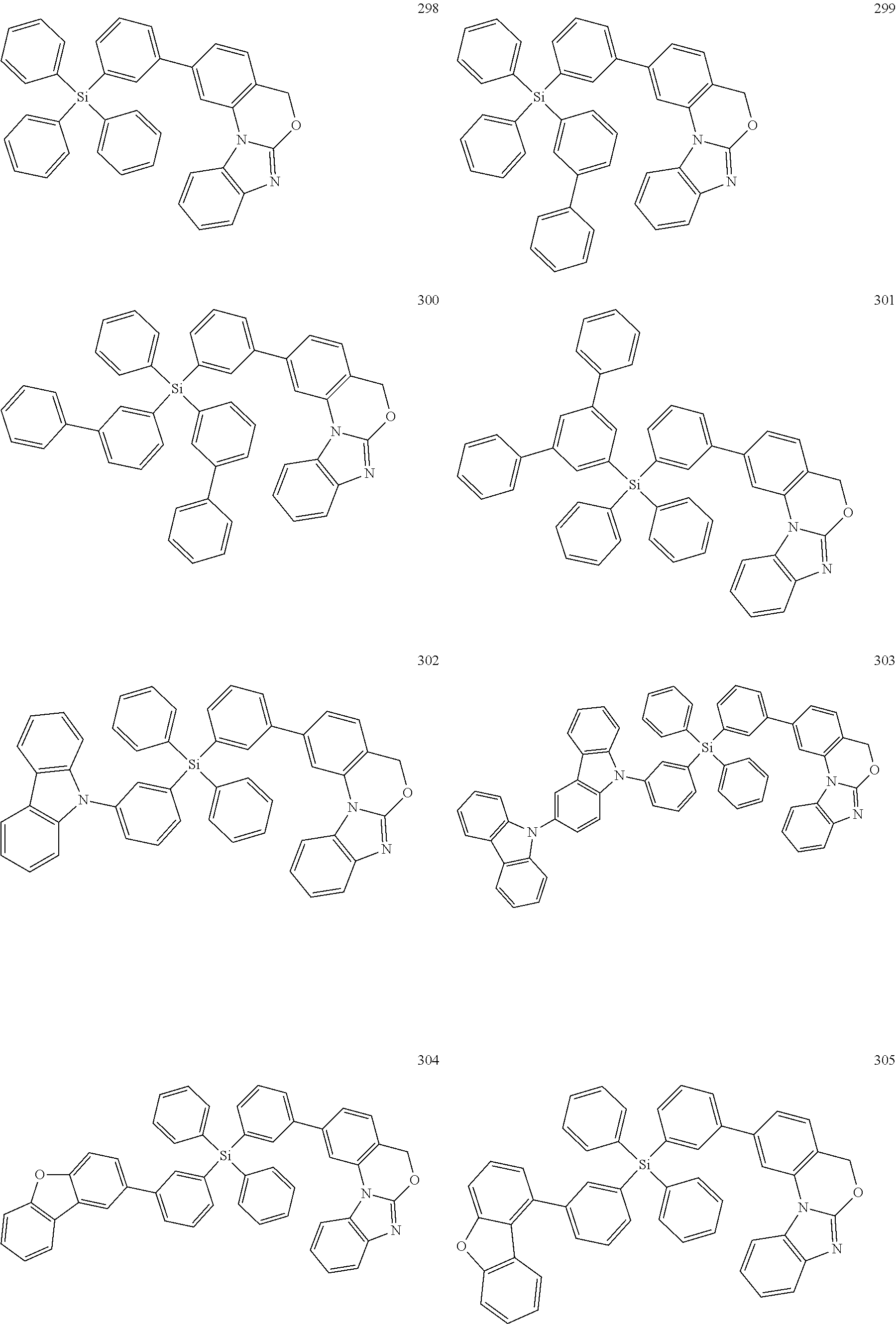 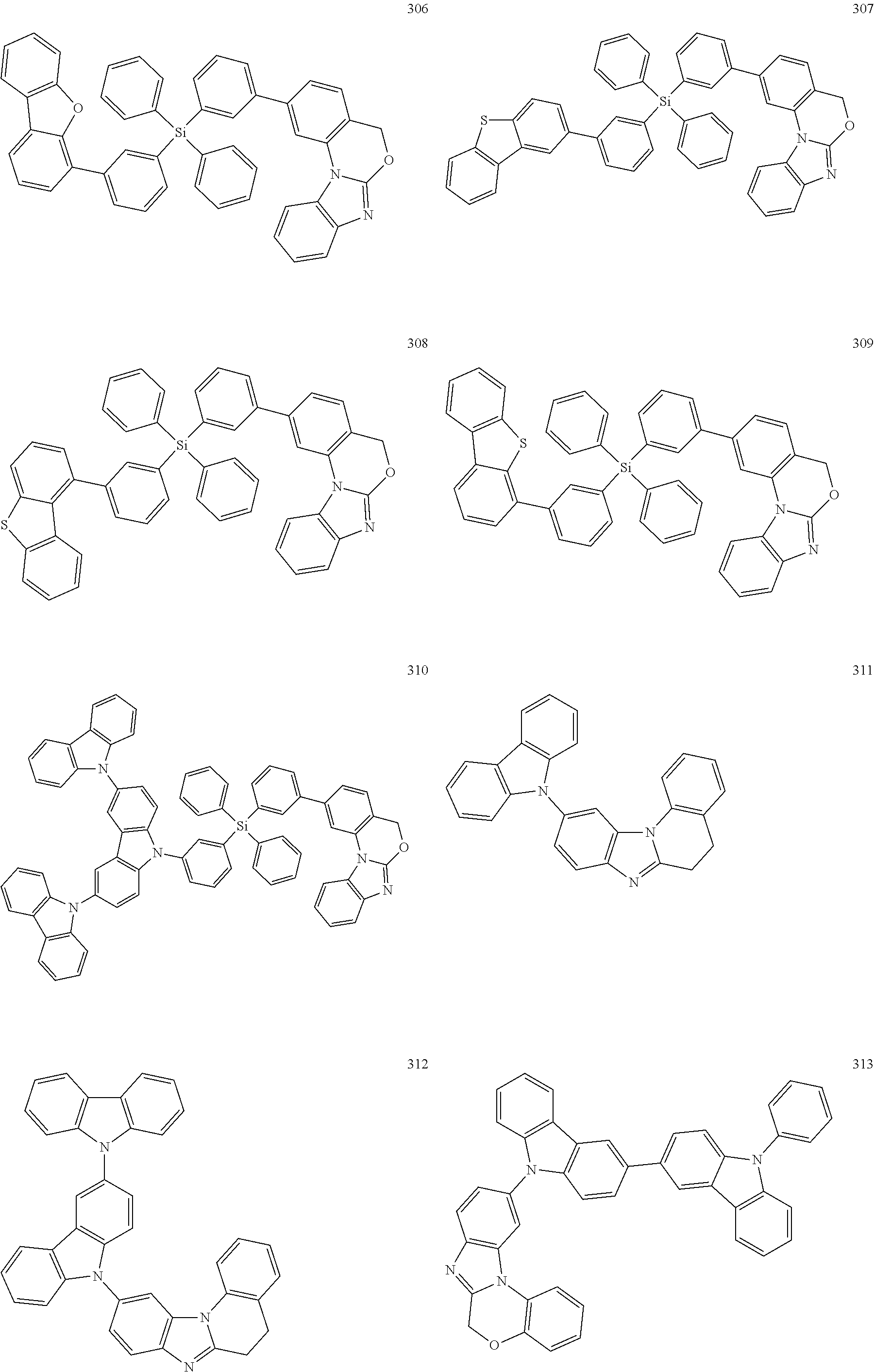 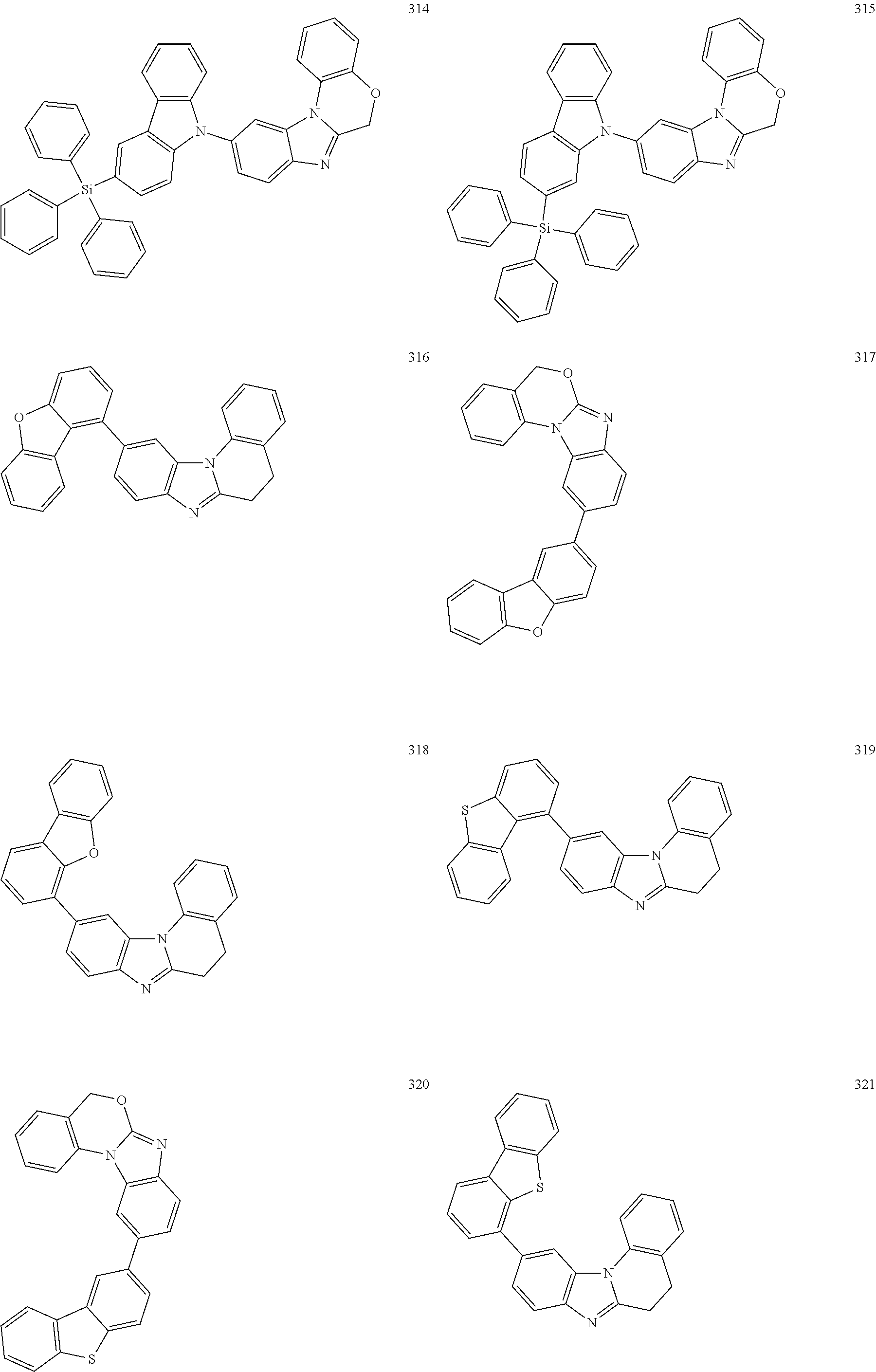 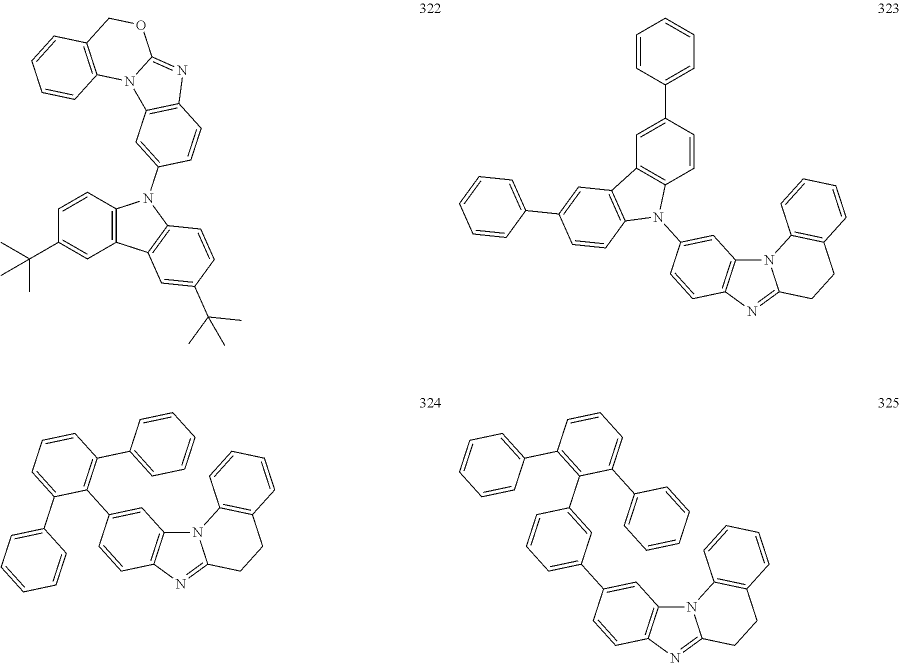 [SMILES: CC(C)(C)c1ccc2c(c1)c1cc(C(C)(C)C)ccc1n2-c1ccc2nc3n(c2c1)-c1ccccc1CO3 ; c1ccc(-c2ccc3c(c2)c2cc(-c4ccccc4)ccc2n3-c2ccc3nc4n(c3c2)-c2ccccc2CC4)cc1 ; c1ccc(-c2cccc(-c3ccccc3)c2-c2ccc3nc4n(c3c2)-c2ccccc2CC4)cc1 ; c1ccc(-c2cccc(-c3ccccc3)c2-c2cccc(-c3ccc4nc5n(c4c3)-c3ccccc3CC5)c2)cc1] 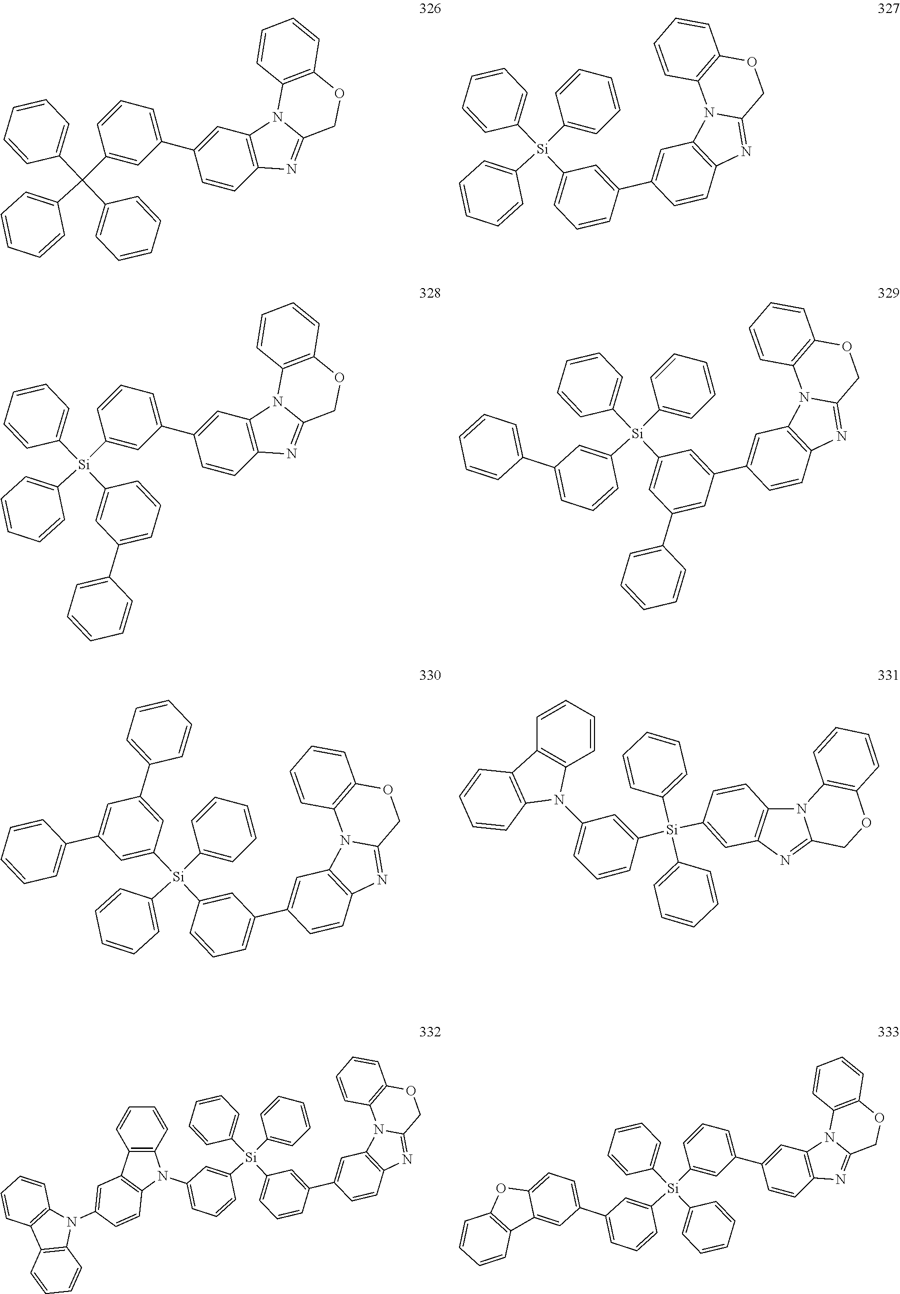 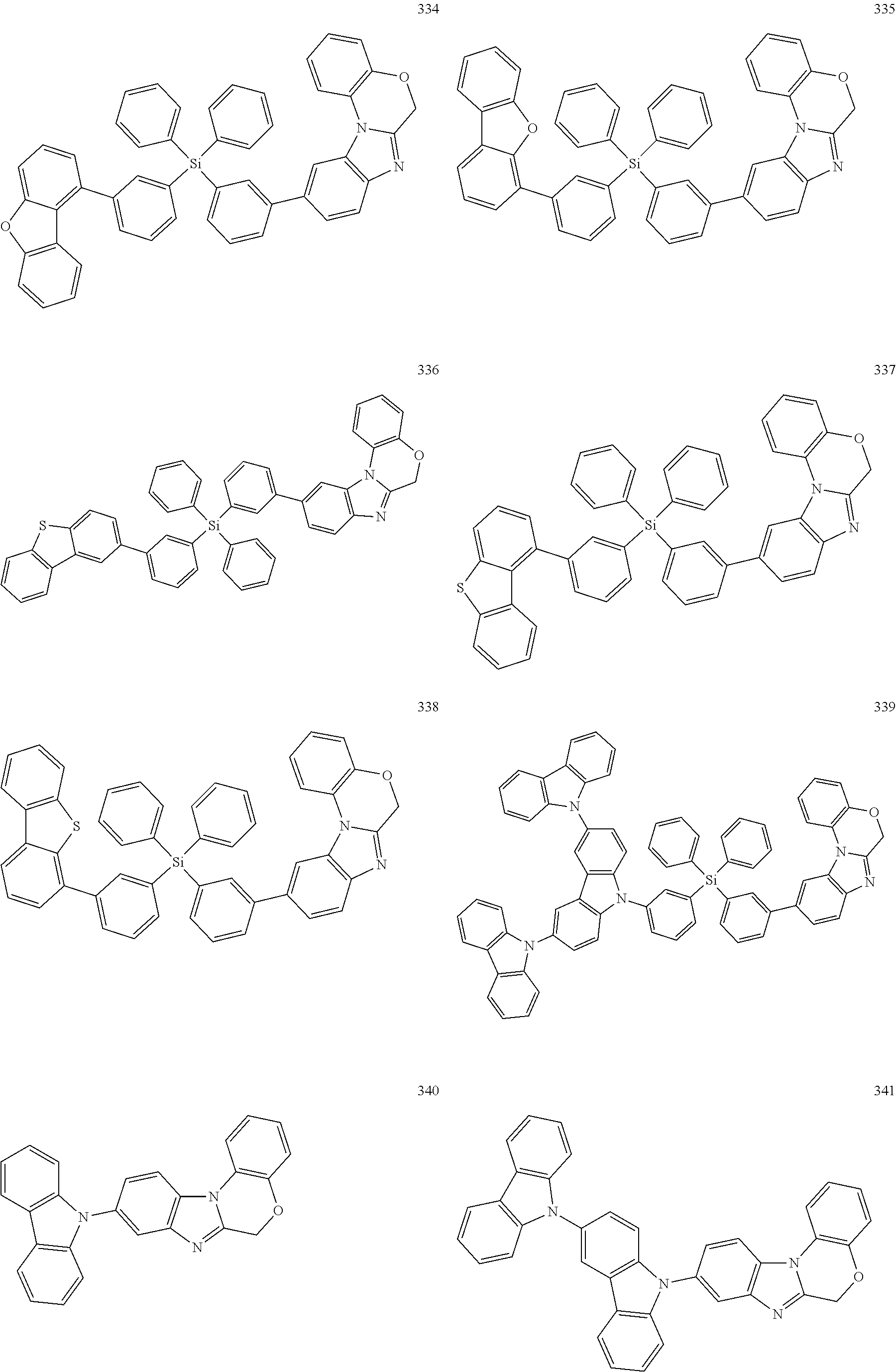 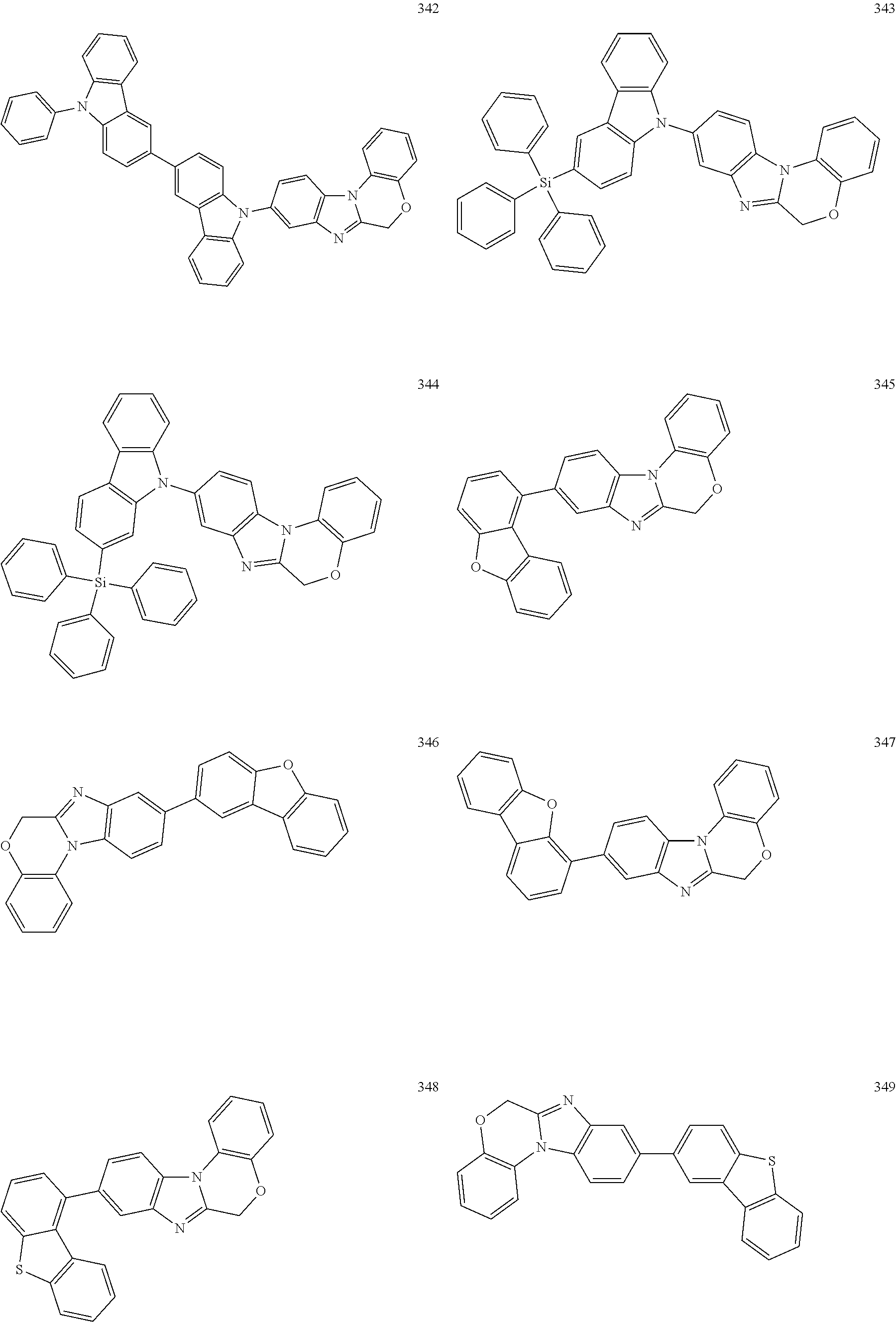 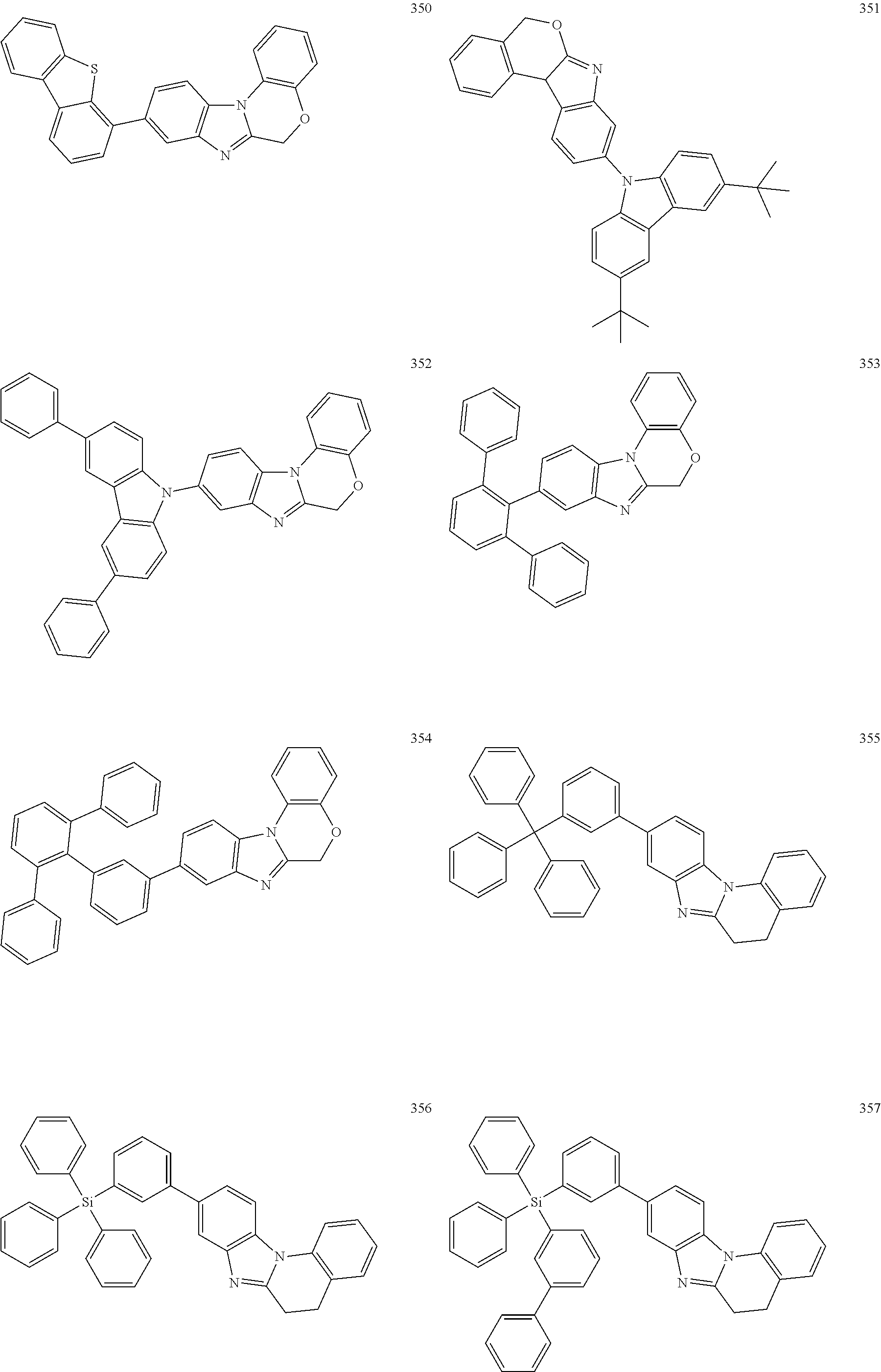 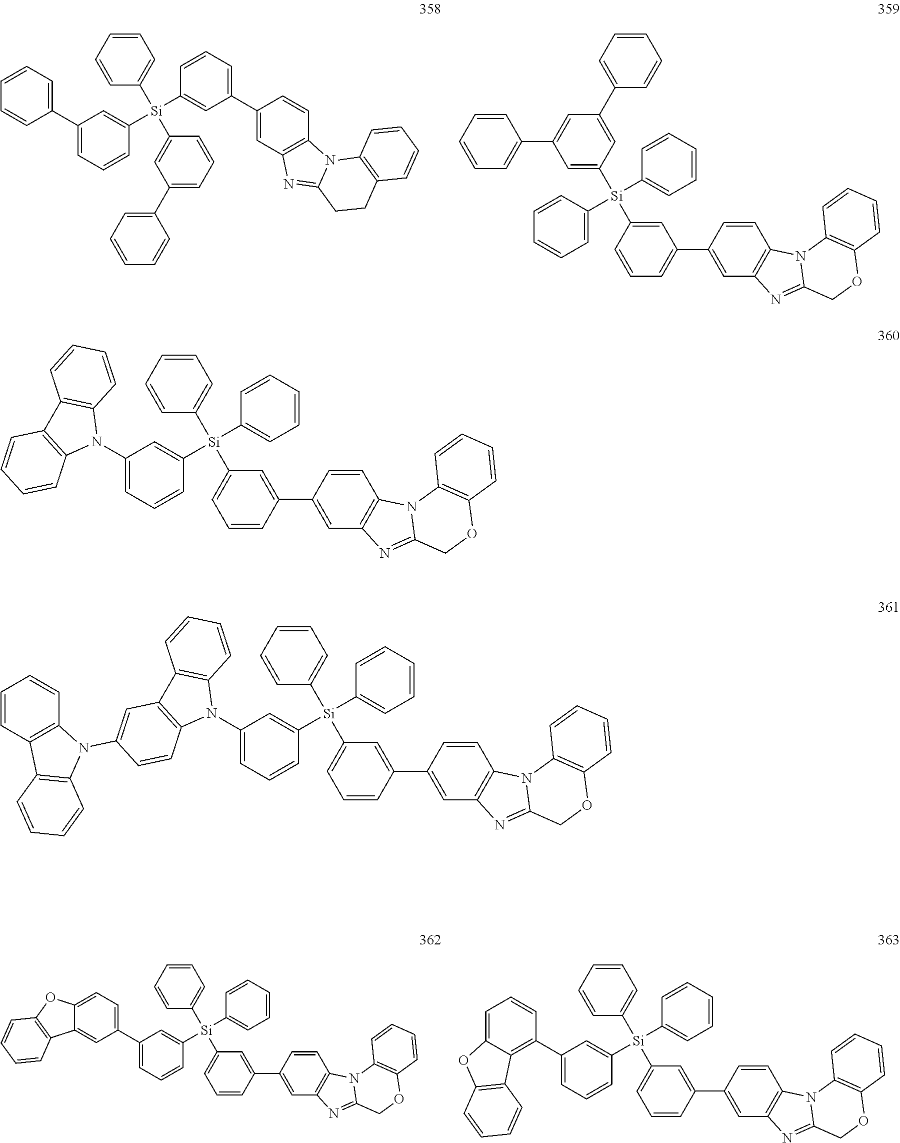 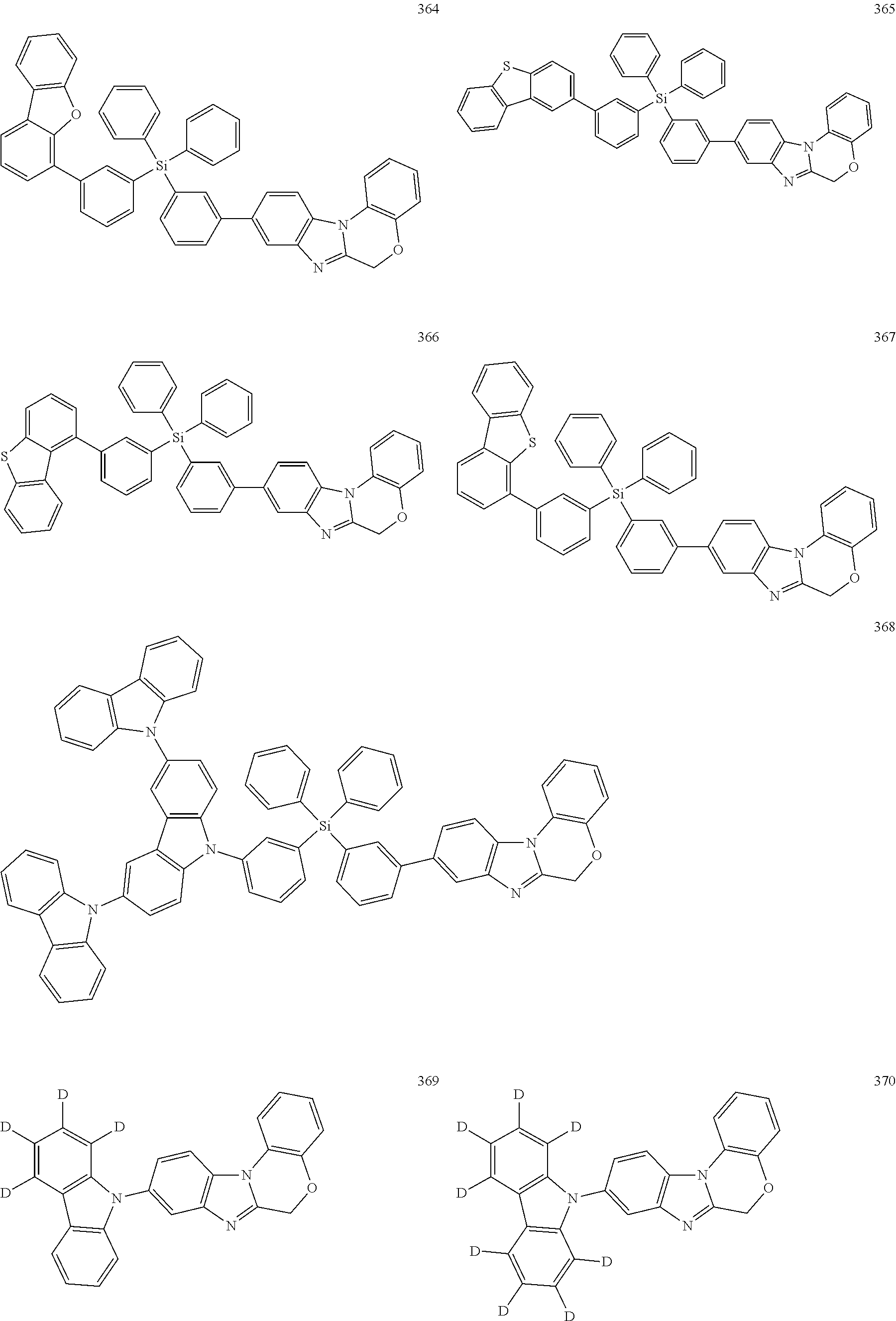 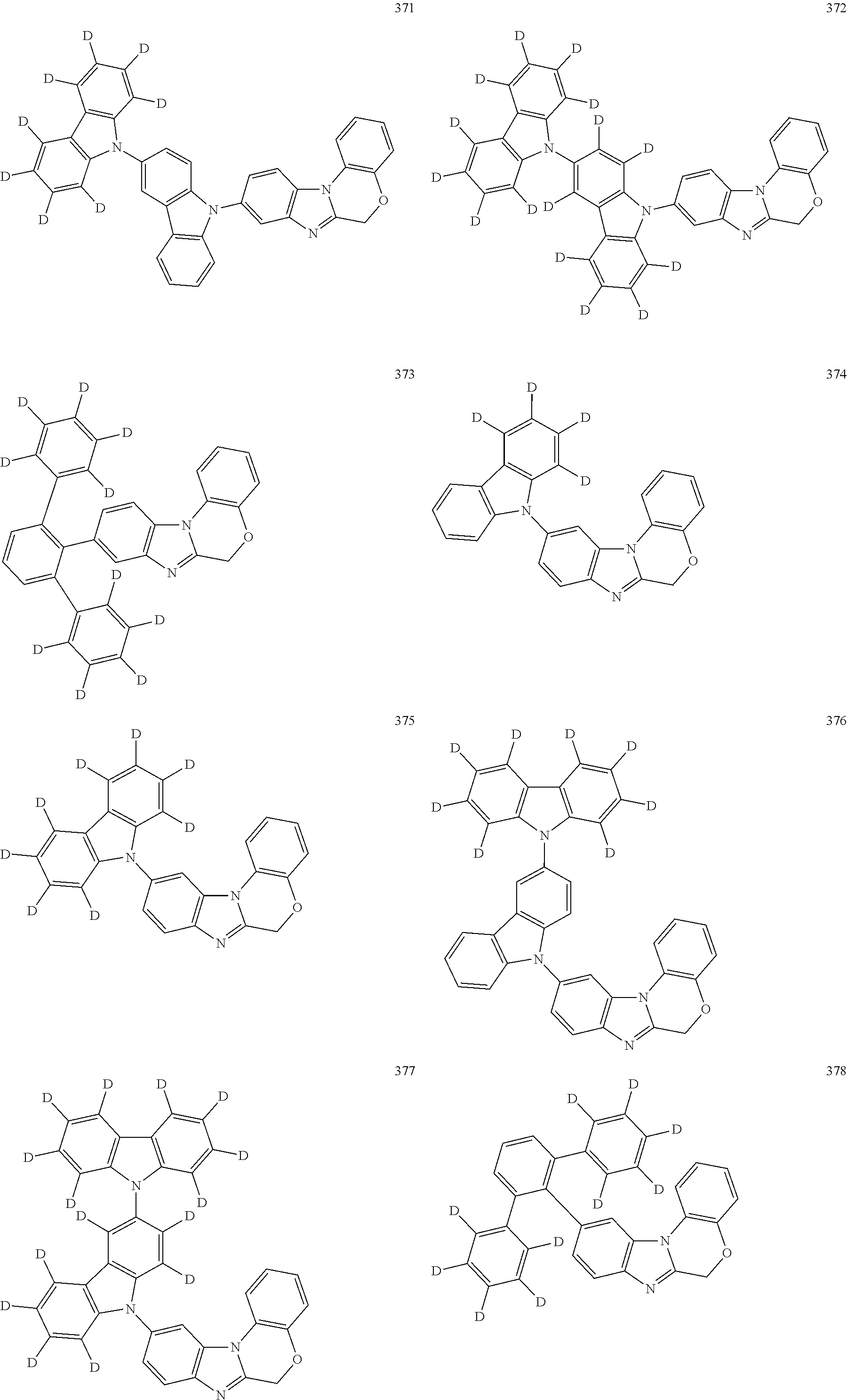 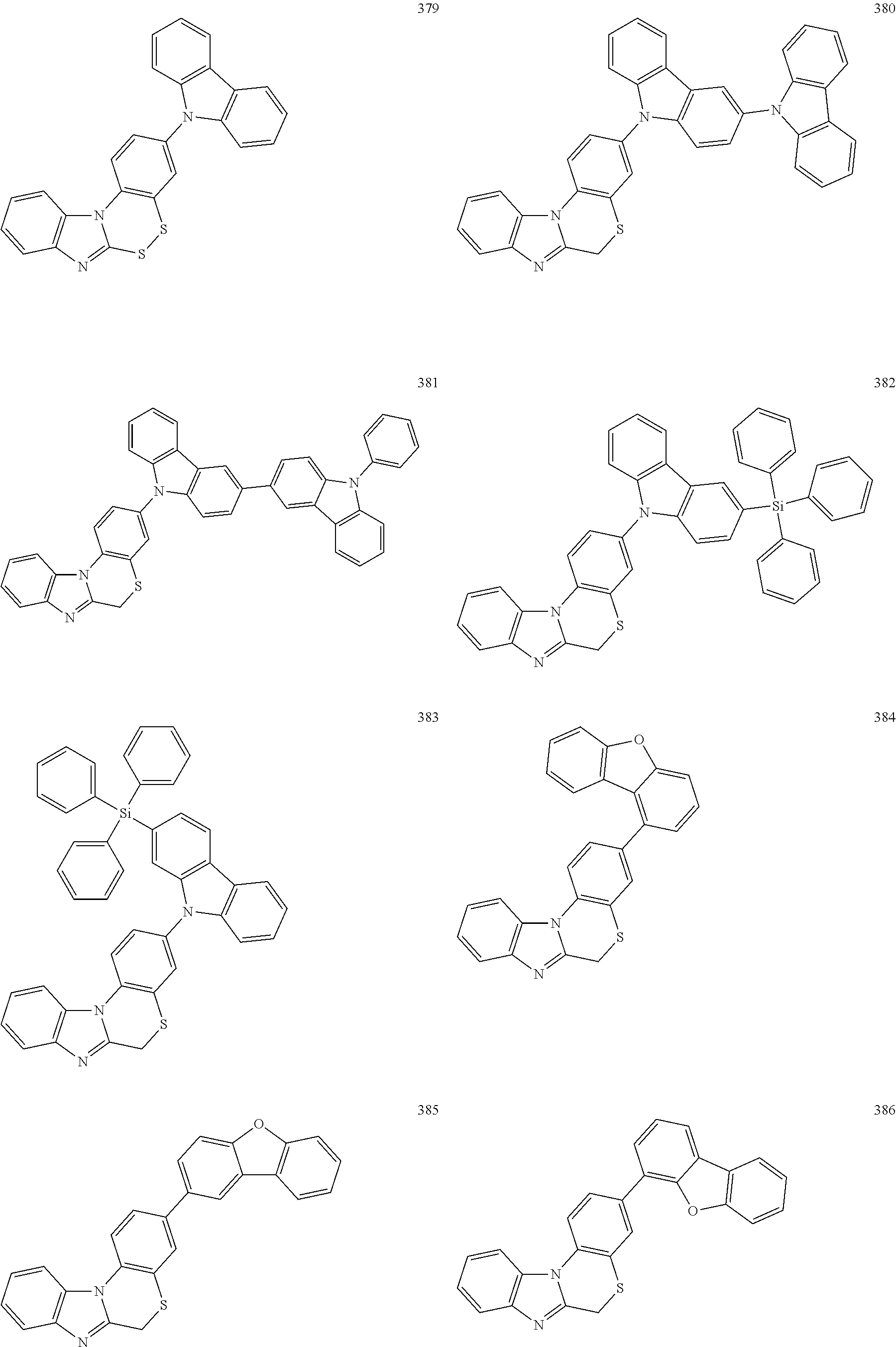 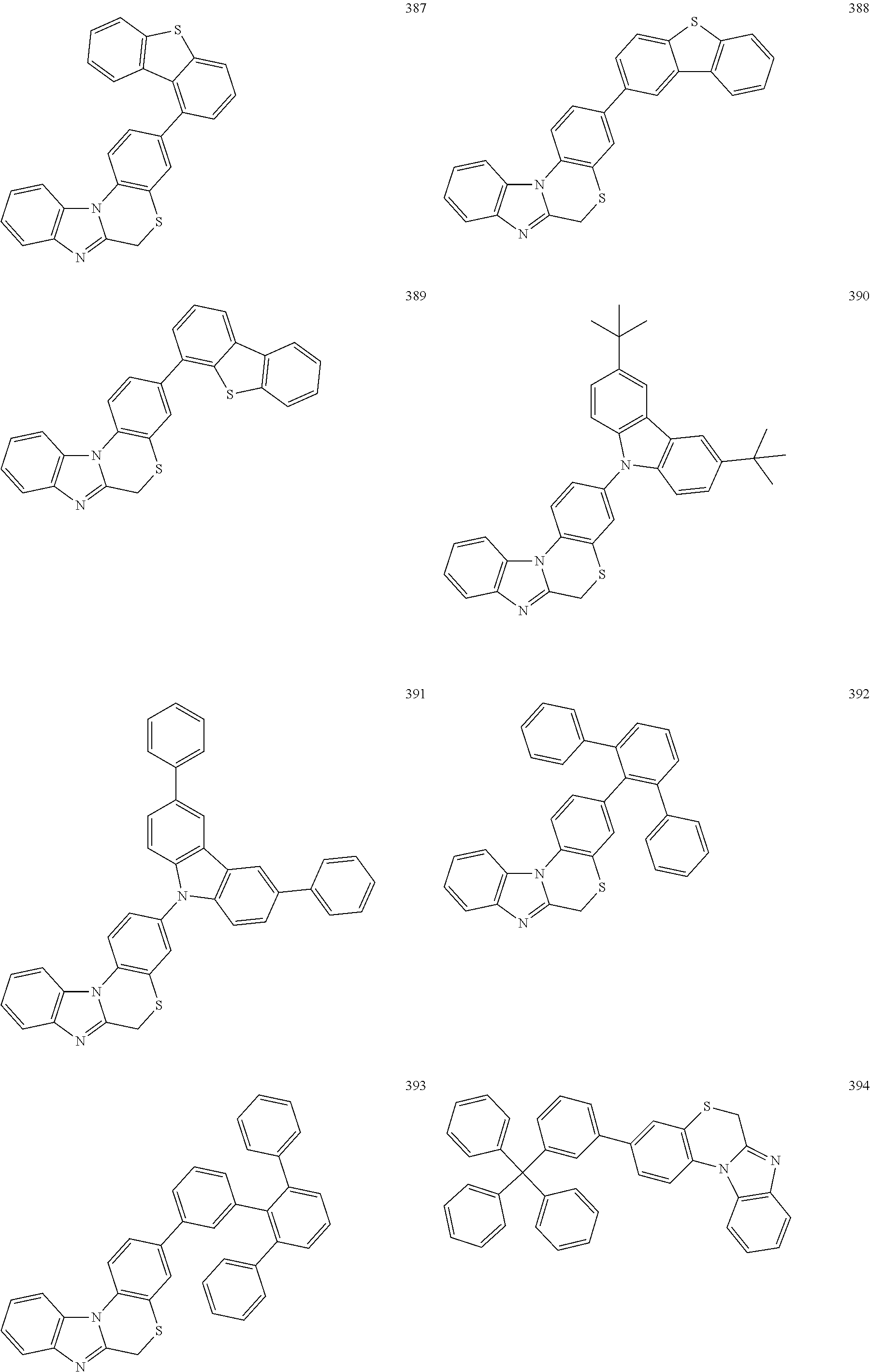 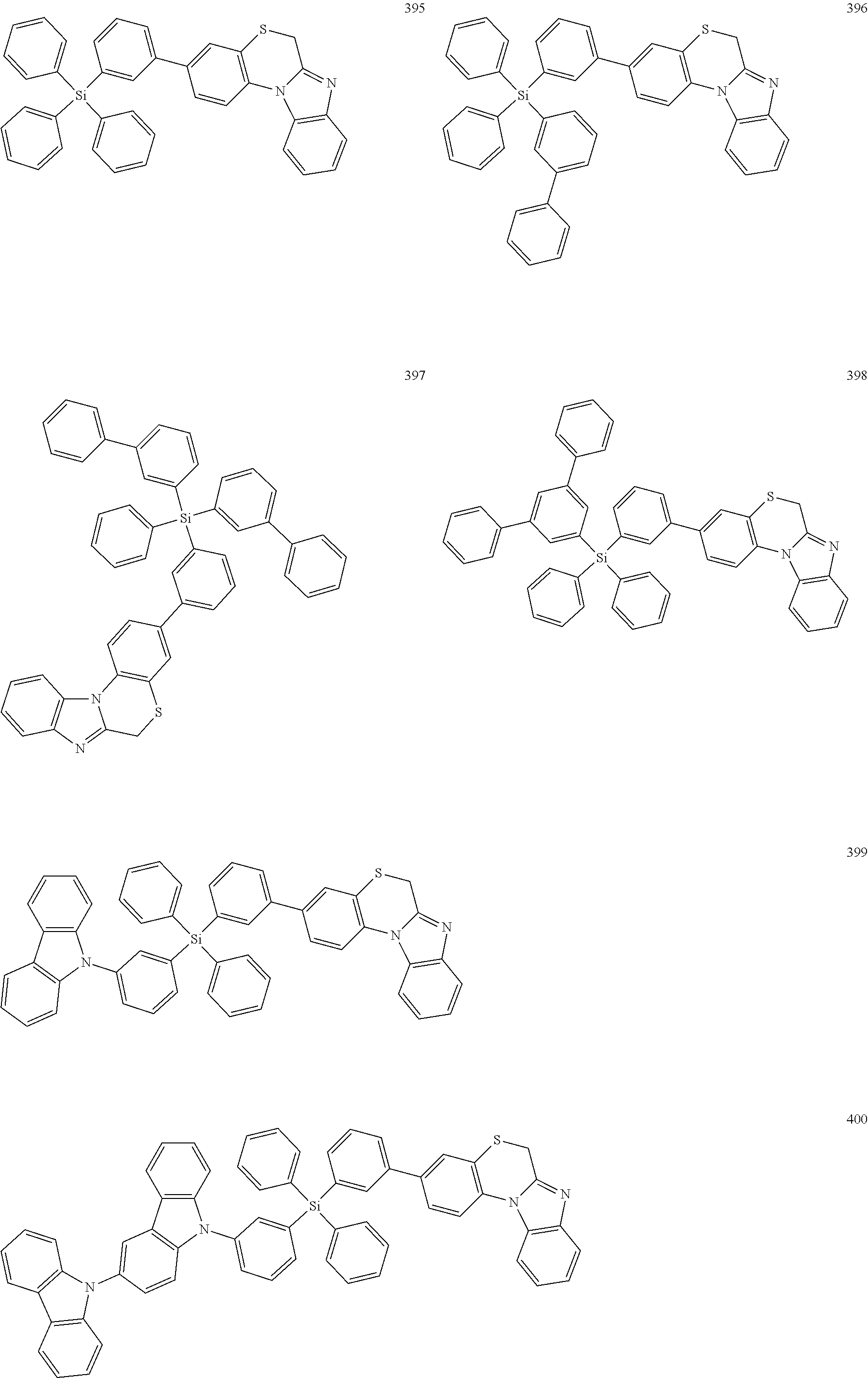 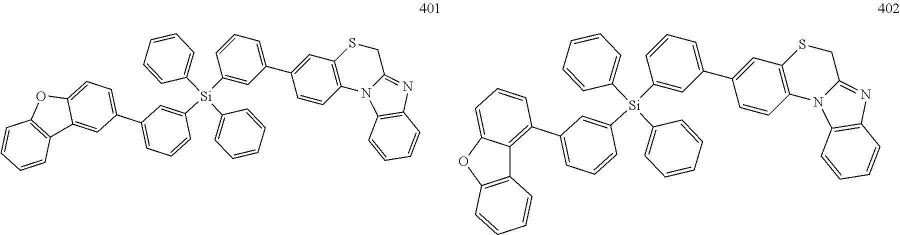 [SMILES: c1ccc([Si](c2ccccc2)(c2cccc(-c3ccc4c(c3)SCc3nc5ccccc5n3-4)c2)c2cccc(-c3ccc4oc5ccccc5c4c3)c2)cc1 ; c1ccc([Si](c2ccccc2)(c2cccc(-c3ccc4c(c3)SCc3nc5ccccc5n3-4)c2)c2cccc(-c3cccc4oc5ccccc5c34)c2)cc1] 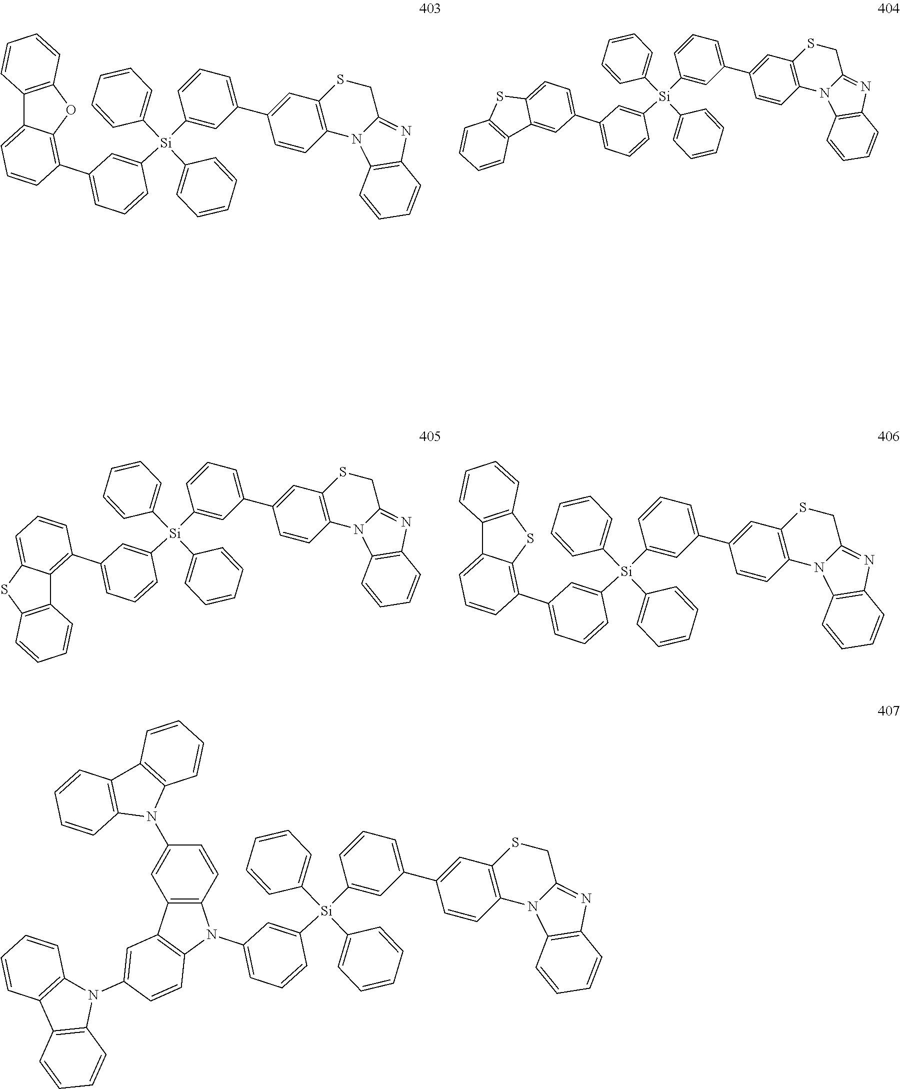 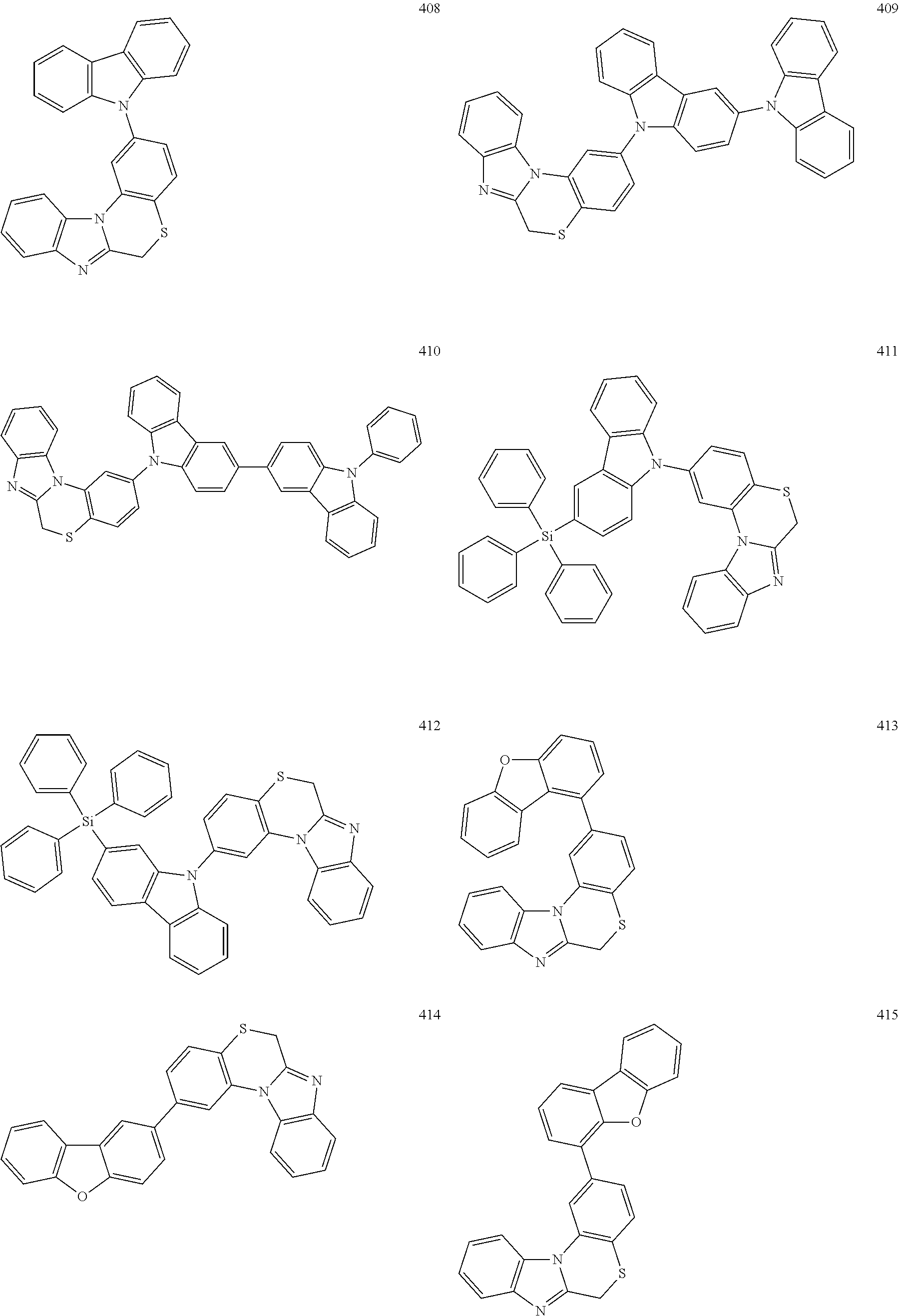 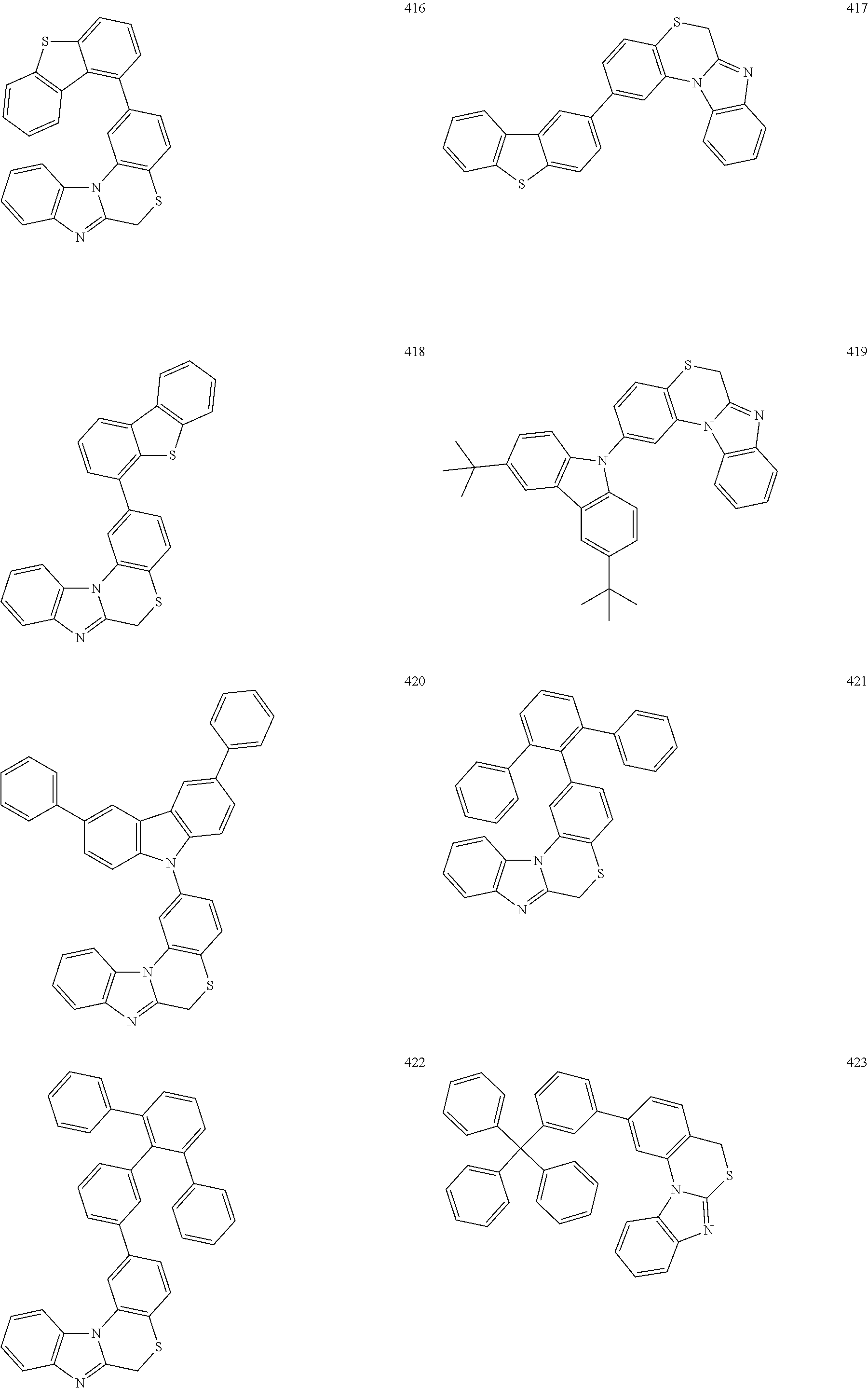 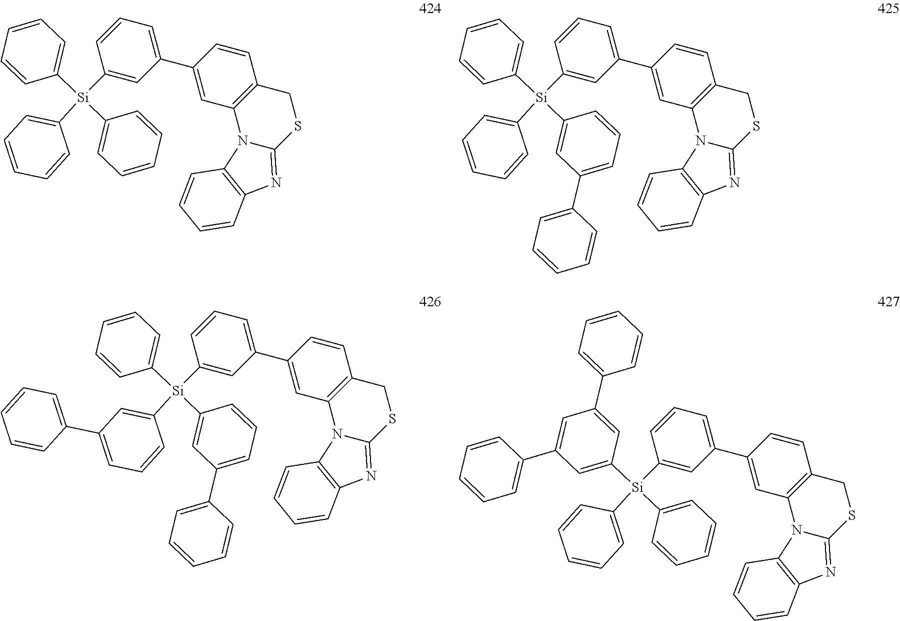 [SMILES: c1ccc([Si](c2ccccc2)(c2ccccc2)c2cccc(-c3ccc4c(c3)-n3c(nc5ccccc53)SC4)c2)cc1 ; c1ccc(-c2cccc([Si](c3ccccc3)(c3ccccc3)c3cccc(-c4ccc5c(c4)-n4c(nc6ccccc64)SC5)c3)c2)cc1 ; c1ccc(-c2cccc([Si](c3ccccc3)(c3cccc(-c4ccccc4)c3)c3cccc(-c4ccc5c(c4)-n4c(nc6ccccc64)SC5)c3)c2)cc1 ; c1ccc(-c2cc(-c3ccccc3)cc([Si](c3ccccc3)(c3ccccc3)c3cccc(-c4ccc5c(c4)-n4c(nc6ccccc64)SC5)c3)c2)cc1] 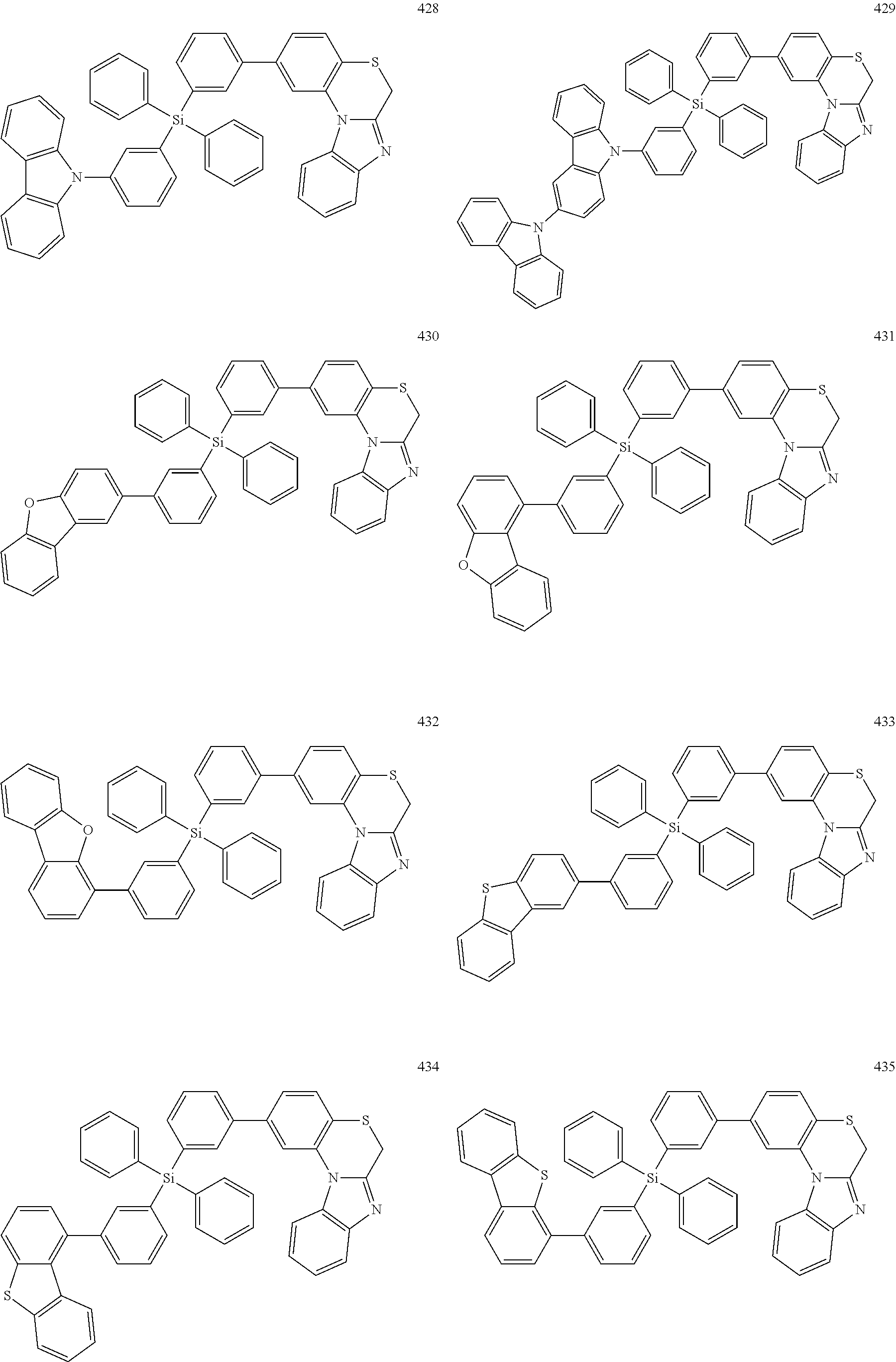 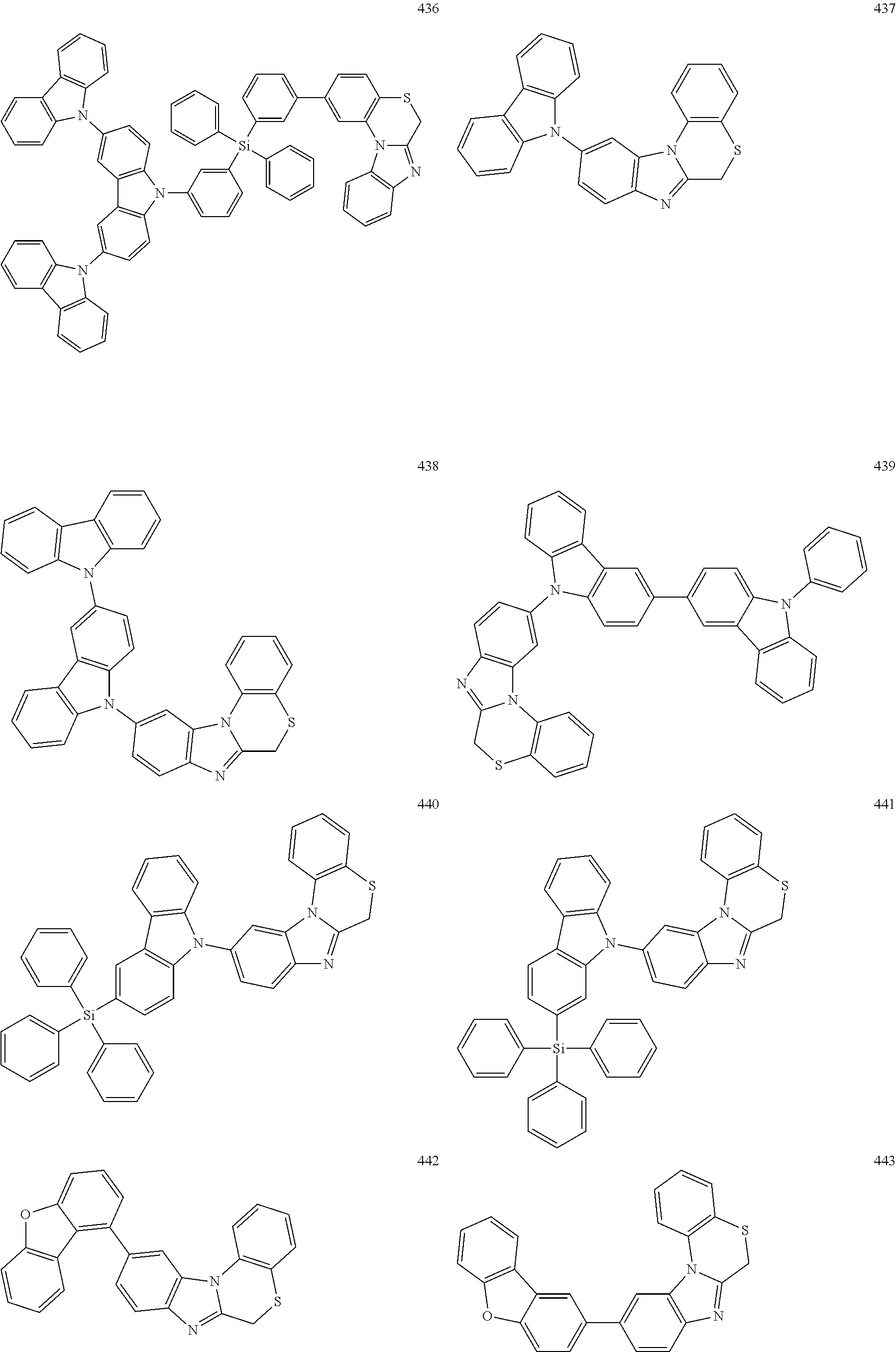 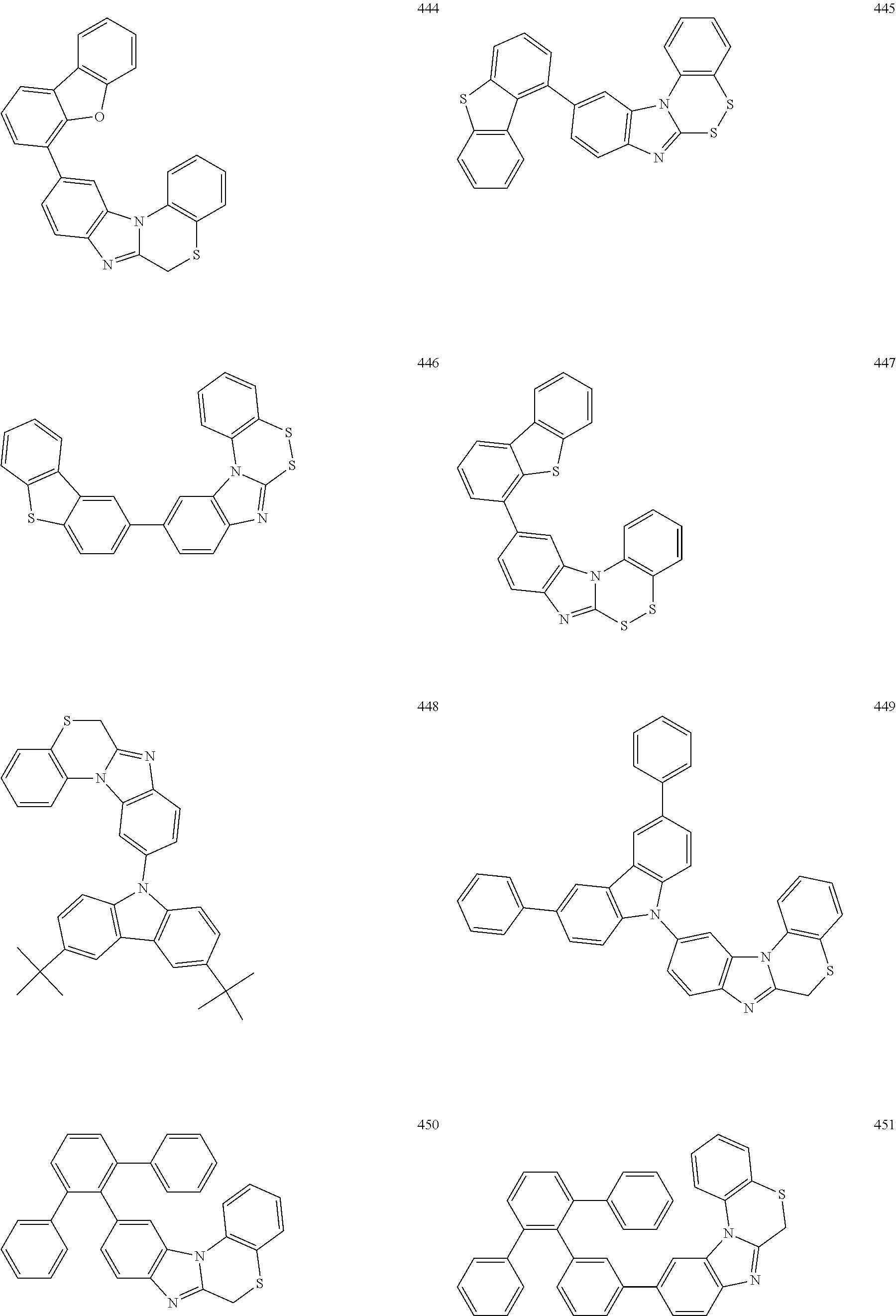 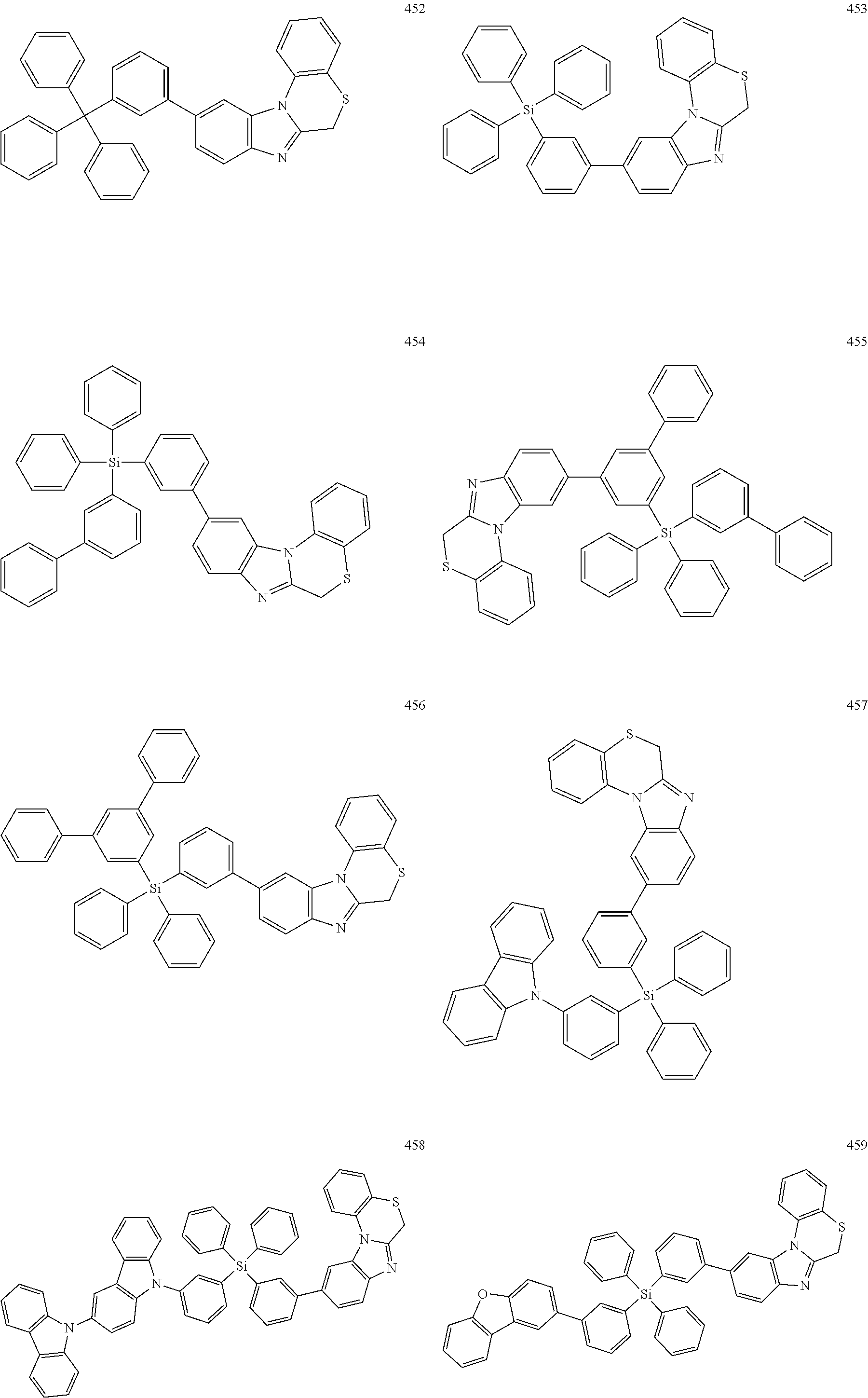 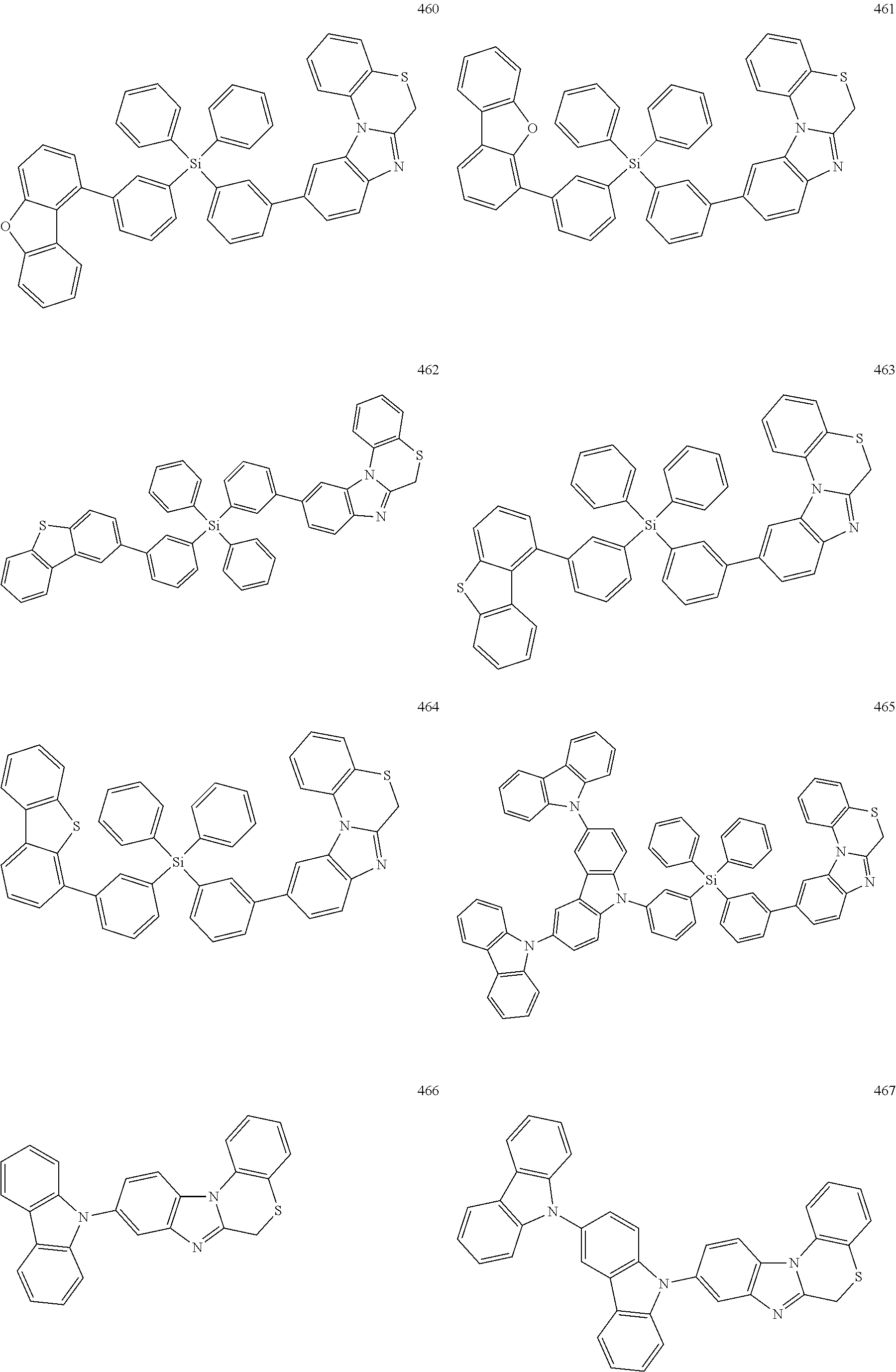 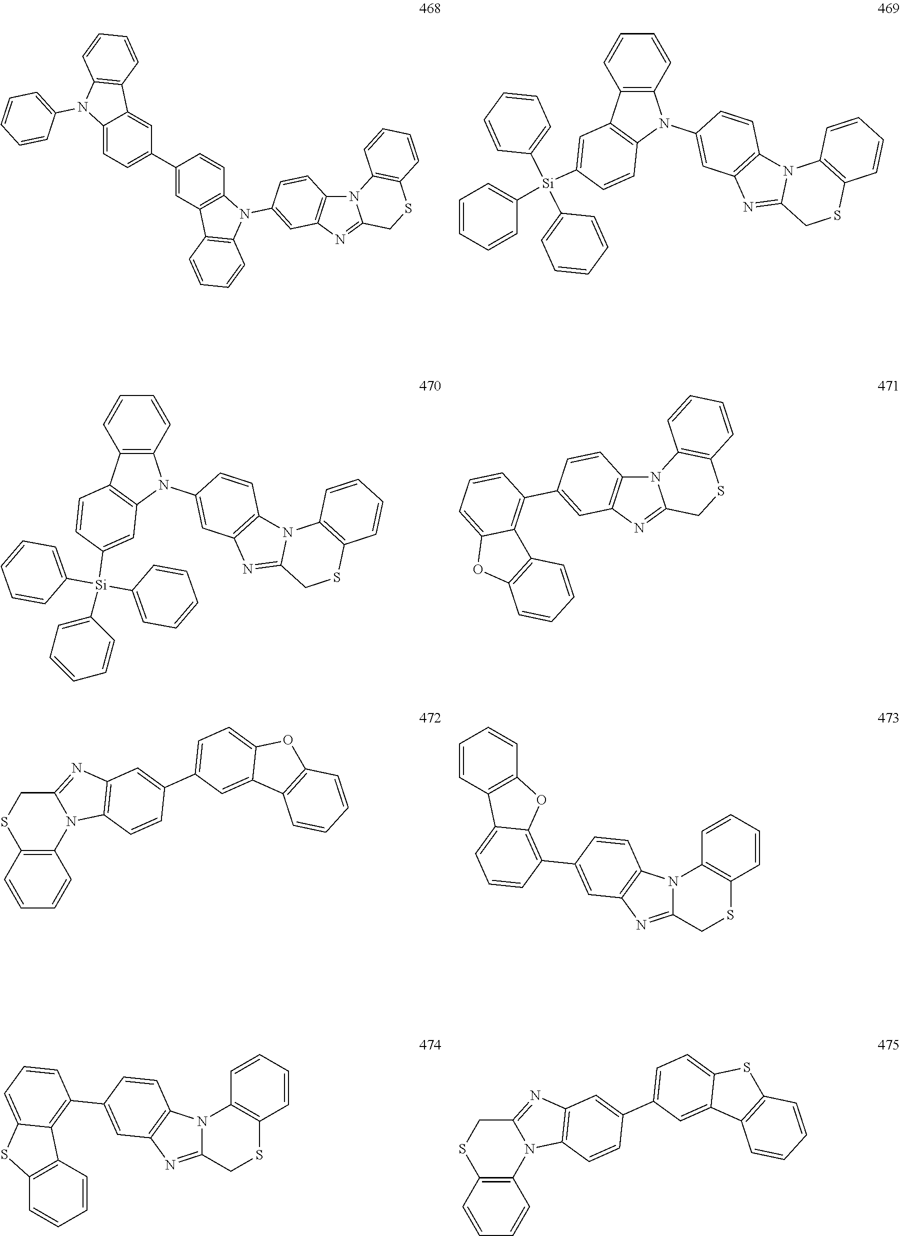 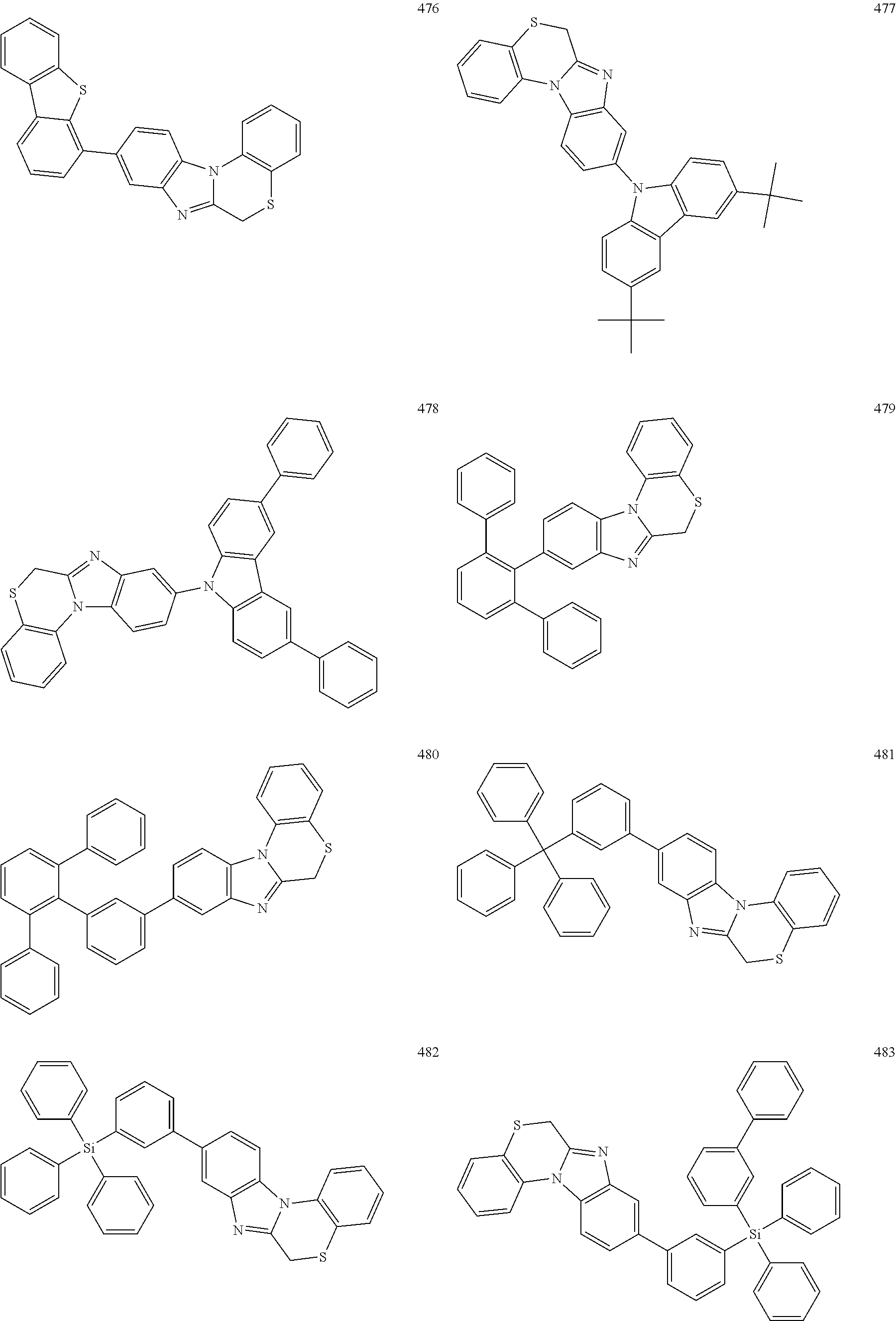 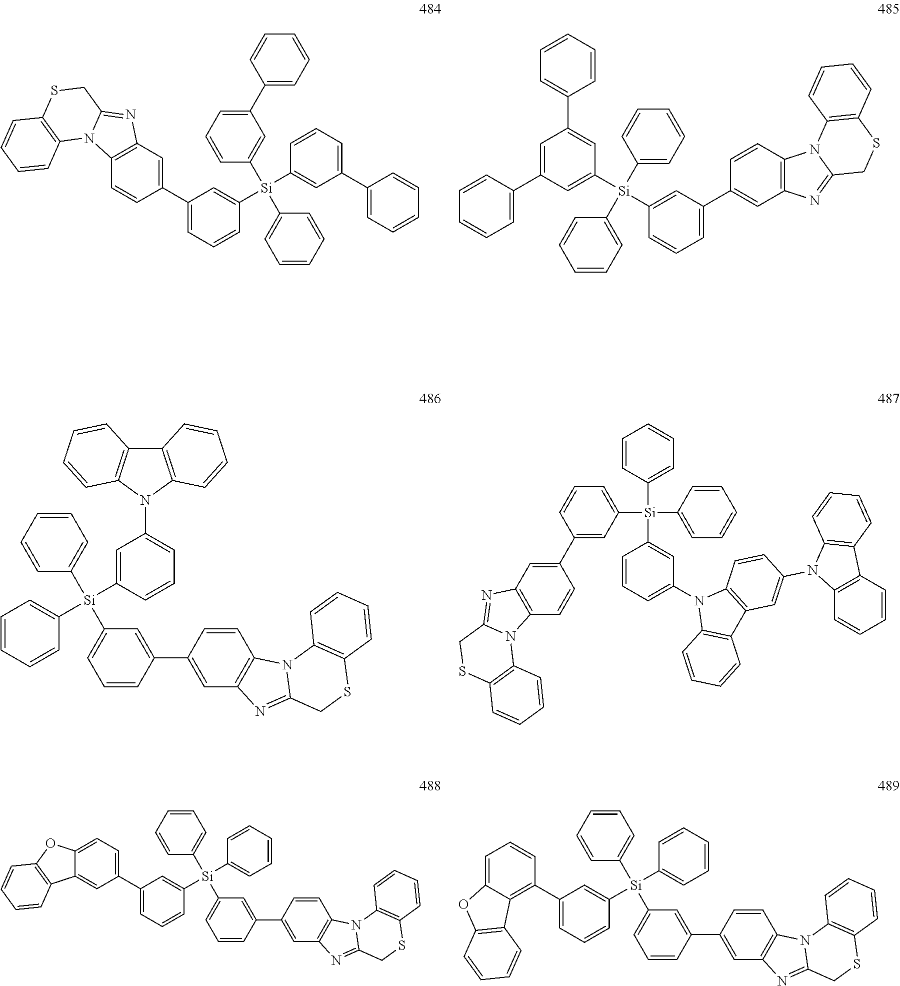 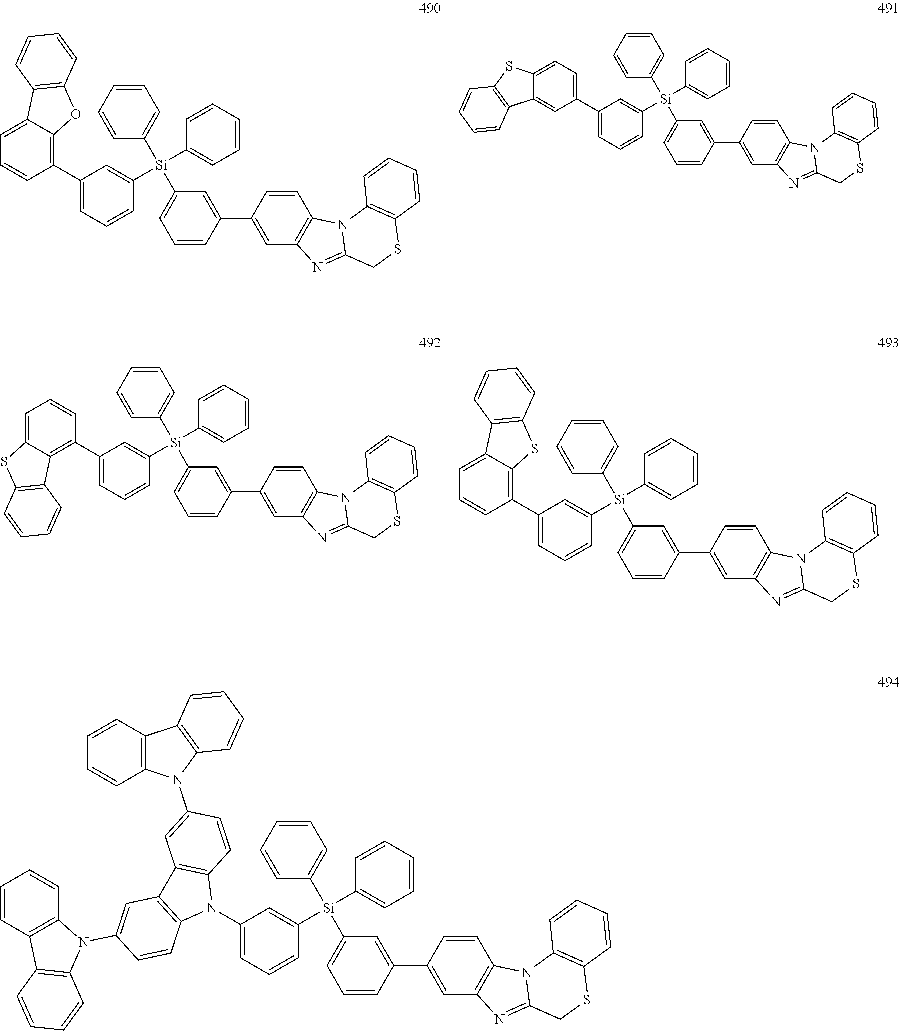 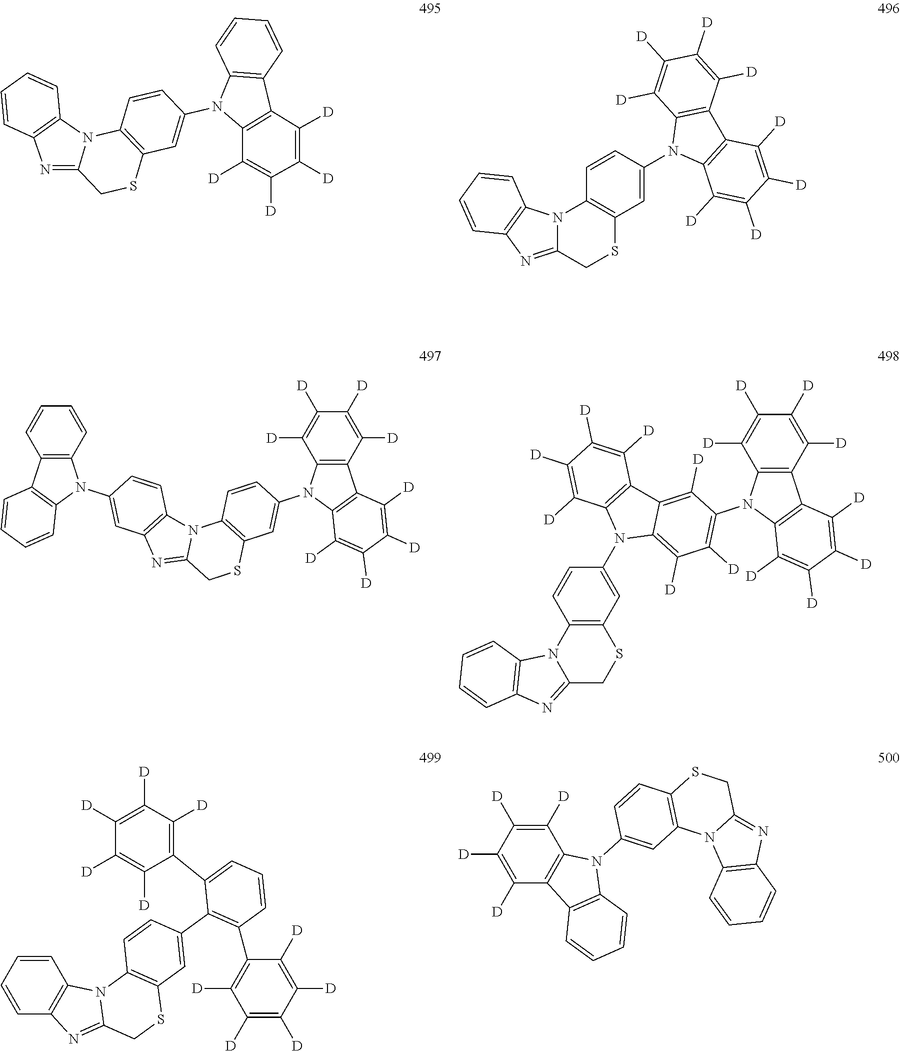 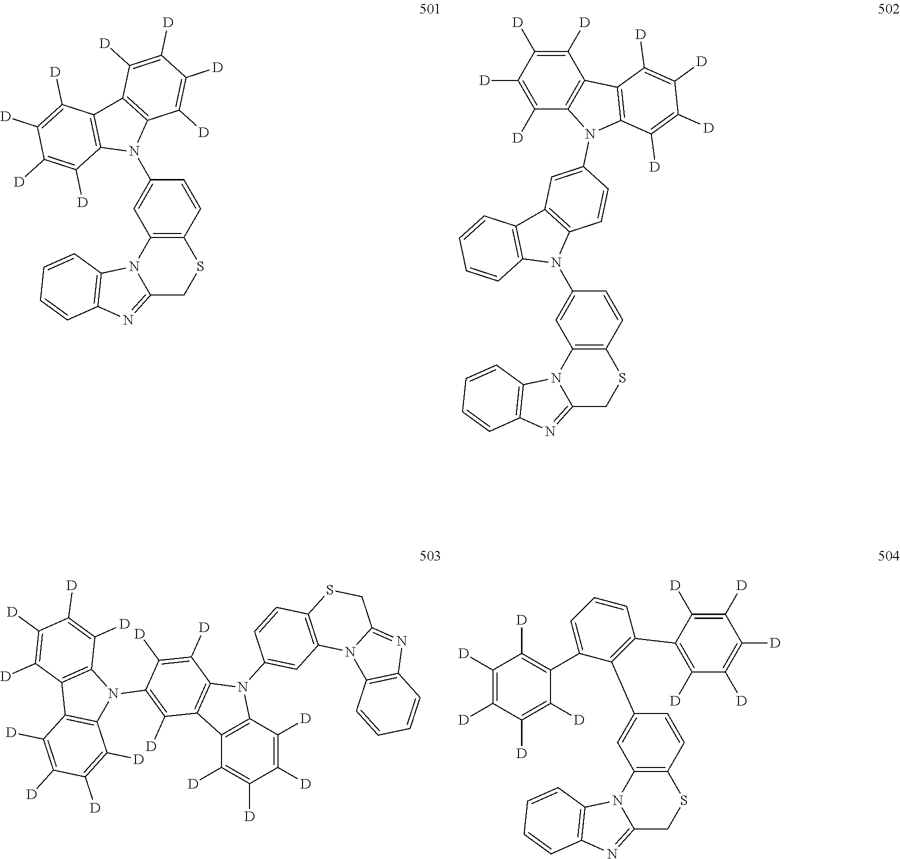 [SMILES: [2H]c1c([2H])c([2H])c2c(c1[2H])c1c([2H])c([2H])c([2H])c([2H])c1n2-c1ccc2c(c1)-n1c(nc3ccccc31)CS2 ; [2H]c1c([2H])c([2H])c2c(c1[2H])c1c([2H])c([2H])c([2H])c([2H])c1n2-c1ccc2c(c1)c1ccccc1n2-c1ccc2c(c1)-n1c(nc3ccccc31)CS2 ; [2H]c1c([2H])c([2H])c2c(c1[2H])c1c([2H])c(-n3c4c([2H])c([2H])c([2H])c([2H])c4c4c([2H])c([2H])c([2H])c([2H])c43)c([2H])c([2H])c1n2-c1ccc2c(c1)-n1c(nc3ccccc31)CS2 ; [2H]c1c([2H])c([2H])c(-c2cccc(-c3c([2H])c([2H])c([2H])c([2H])c3[2H])c2-c2ccc3c(c2)-n2c(nc4ccccc42)CS3)c([2H])c1[2H]]
